SEQUENCE LISTING (569 sequences)

SEQ ID NO: 1 - protein, 468 aa, synthetic construct
  REGION 1..468: note = Human PAC1
MAGVVHVSLAALLLLPMAPAMHSDCIFKKEQAMCLEKIQRANELMGFNDSSPGCPGMWDNITCWKPAHVGEMVLVSCPELFRIFNPDQVWETETIGESDFGDSNSLDLSDMGVVSRNCTEDGWSEPFPHYFDACGFDEYESETGDQDYYYLSVKALYTVGYSTSLVTLTTAMVILCRFRKLHCTRNFIHMNLFVSFMLRAISVFIKDWILYAEQDSNHCFISTVECKAVMVFFHYCVVSNYFWLFIEGLYLFTLLVETFFPERRYFYWYTIIGWGTPTVCVTVWATLRLYFDDTGCWDMNDSTALWWVIKGPVVGSIMVNFVLFIGIIVILVQKLQSPDMGGNESSIYLRLARSTLLLIPLFGIHYTVFAFSPENVSKRERLVFELGLGSFQGFVVAVLYCFLNGEVQAEIKRKWRSWKVNRYFAVDFKHRHPSLASSGVNGGTQLSILSKSSSQIRMSGLPADNLAT

SEQ ID NO: 2 - protein, 226 aa, synthetic construct
  REGION 1..226: note = Human PAC1 ECD
QVQLVESGGGVVQPGRSLRLSCAASGFTFSRFAMHWVRQAPGKGLEWVAVISYDGGNKYYAESVKGRFTISRDNSKNTLYLQMNSLRAEDTALFYCARGYDVLTGYPDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKGDEVD

SEQ ID NO: 3 - protein, 214 aa, synthetic construct
  REGION 1..214: note = Human PAC1 ECD
DIQLTQSPSFLSASVGDRVTITCRASQSIGRSLHWYQQKPGKAPKLLIKYASQSLSGVPSRFSGSGSGTEFTLTISSLQPEDFATYYCHQSSRLPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 105 aa, synthetic construct
  REGION 1..105: note = Human PAC1 ECD
GSMAHSDGIFKKEQAMCLEKIQRANELMGFNDSSPGCPGMWDNITCWKPAHVGEMVLVSCPELFRIFNPDQDMGVVSRNCTEDGWSEPFPHYFDACGFDEYESET

SEQ ID NO: 5 - protein, 11 aa, synthetic construct
  REGION 1..11: note = Synthetic polypeptide
RASQSVGRSLH

SEQ ID NO: 6 - protein, 11 aa, synthetic construct
  REGION 1..11: note = Synthetic polypeptide
RASQSIGRSLH

SEQ ID NO: 7 - protein, 11 aa, synthetic construct
  REGION 1..11: note = Synthetic polypeptide
RASKSVGRSLH

SEQ ID NO: 8 - protein, 11 aa, synthetic construct
  REGION 1..11: note = Synthetic polypeptide
RASQSVWRSLH

SEQ ID NO: 9 - protein, 11 aa, synthetic construct
  REGION 1..11: note = Synthetic polypeptide
RASQSVGRNLH

SEQ ID NO: 10 - protein, 11 aa, synthetic construct
  REGION 1..11: note = Synthetic polypeptide
RASKSVWRSLH

SEQ ID NO: 11 - protein, 11 aa, synthetic construct
  REGION 1..11: note = Synthetic polypeptide
RASKSVGRNLH

SEQ ID NO: 12 - protein, 11 aa, synthetic construct
  REGION 1..11: note = Synthetic polypeptide
RASKSVGWSLH

SEQ ID NO: 13 - protein, 11 aa, synthetic construct
  REGION 1..11: note = Synthetic polypeptide
RASKSVGYSLH

SEQ ID NO: 14 - protein, 11 aa, synthetic construct
  REGION 1..11: note = Synthetic polypeptide
RASKAVGWSLH

SEQ ID NO: 15 - protein, 11 aa, synthetic construct
  REGION 1..11: note = Synthetic polypeptide
RASKSVGQSLH

SEQ ID NO: 16 - protein, 11 aa, synthetic construct
  REGION 1..11: note = Synthetic polypeptide
RASRSVGLALH

SEQ ID NO: 17 - protein, 11 aa, synthetic construct
  REGION 1..11: note = Synthetic polypeptide
RASQSISRYLN

SEQ ID NO: 18 - protein, 11 aa, synthetic construct
  REGION 1..11: note = Synthetic polypeptide
RASQYIVRYLN

SEQ ID NO: 19 - protein, 11 aa, synthetic construct
  REGION 1..11: note = Synthetic polypeptide
RASQTIVRYLN

SEQ ID NO: 20 - protein, 11 aa, synthetic construct
  REGION 1..11: note = Synthetic polypeptide
RASQKIARYLV

SEQ ID NO: 21 - protein, 11 aa, synthetic construct
  REGION 1..11: note = Synthetic polypeptide
RASQPIAQYLN

SEQ ID NO: 22 - protein, 11 aa, synthetic construct
  REGION 1..11: note = Synthetic polypeptide
RASQPISRYLS

SEQ ID NO: 23 - protein, 11 aa, synthetic construct
  REGION 1..11: note = Synthetic polypeptide
RASQQIARYLN

SEQ ID NO: 24 - protein, 11 aa, synthetic construct
  REGION 1..11: note = Synthetic polypeptide
RASQRIARYLN

SEQ ID NO: 25 - protein, 11 aa, synthetic construct
  REGION 1..11: note = Synthetic polypeptide
RASQMIARYLN

SEQ ID NO: 26 - protein, 7 aa, synthetic construct
  REGION 1..7: note = Synthetic polypeptide
YASQSLS

SEQ ID NO: 27 - protein, 7 aa, synthetic construct
  REGION 1..7: note = Synthetic polypeptide
AASSLQS

SEQ ID NO: 28 - protein, 7 aa, synthetic construct
  REGION 1..7: note = Synthetic polypeptide
AGQRLQS

SEQ ID NO: 29 - protein, 7 aa, synthetic construct
  REGION 1..7: note = Synthetic polypeptide
AAHHLQS

SEQ ID NO: 30 - protein, 7 aa, synthetic construct
  REGION 1..7: note = Synthetic polypeptide
AANMLQS

SEQ ID NO: 31 - protein, 7 aa, synthetic construct
  REGION 1..7: note = Synthetic polypeptide
ASYNLQS

SEQ ID NO: 32 - protein, 7 aa, synthetic construct
  REGION 1..7: note = Synthetic polypeptide
AGRYLQS

SEQ ID NO: 33 - protein, 7 aa, synthetic construct
  REGION 1..7: note = Synthetic polypeptide
AGRILQS

SEQ ID NO: 34 - protein, 7 aa, synthetic construct
  REGION 1..7: note = Synthetic polypeptide
AGSILQS

SEQ ID NO: 35 - protein, 7 aa, synthetic construct
  REGION 1..7: note = Synthetic polypeptide
GARNLQS

SEQ ID NO: 36 - protein, 9 aa, synthetic construct
  REGION 1..9: note = Synthetic polypeptide
HQSSRLPFT

SEQ ID NO: 37 - protein, 9 aa, synthetic construct
  REGION 1..9: note = Synthetic polypeptide
HQSSMLPFT

SEQ ID NO: 38 - protein, 9 aa, synthetic construct
  REGION 1..9: note = Synthetic polypeptide
HQSSFLPFT

SEQ ID NO: 39 - protein, 9 aa, synthetic construct
  REGION 1..9: note = Synthetic polypeptide
QQSYSPPFT

SEQ ID NO: 40 - protein, 9 aa, synthetic construct
  REGION 1..9: note = Synthetic polypeptide
QQAIGMPYT

SEQ ID NO: 41 - protein, 9 aa, synthetic construct
  REGION 1..9: note = Synthetic polypeptide
QQAIQEPYT

SEQ ID NO: 42 - protein, 9 aa, synthetic construct
  REGION 1..9: note = Synthetic polypeptide
QQAIINPYT

SEQ ID NO: 43 - protein, 9 aa, synthetic construct
  REGION 1..9: note = Synthetic polypeptide
QQAINQPYT

SEQ ID NO: 44 - protein, 9 aa, synthetic construct
  REGION 1..9: note = Synthetic polypeptide
QQSIQQPYT

SEQ ID NO: 45 - protein, 9 aa, synthetic construct
  REGION 1..9: note = Synthetic polypeptide
QQAIQQPYT

SEQ ID NO: 46 - protein, 9 aa, synthetic construct
  REGION 1..9: note = Synthetic polypeptide
QQAIMAPYT

SEQ ID NO: 47 - protein, 9 aa, synthetic construct
  REGION 1..9: note = Synthetic polypeptide
QQAIQNPYT

SEQ ID NO: 48 - protein, 9 aa, synthetic construct
  REGION 1..9: note = Synthetic polypeptide
QQAIVQPYT

SEQ ID NO: 49 - protein, 9 aa, synthetic construct
  REGION 1..9: note = Synthetic polypeptide
QQAISIPYT

SEQ ID NO: 50 - protein, 9 aa, synthetic construct
  REGION 1..9: note = Synthetic polypeptide
QQAIIQPYT

SEQ ID NO: 51 - protein, 9 aa, synthetic construct
  REGION 1..9: note = Synthetic polypeptide
QQSIQTPYT

SEQ ID NO: 52 - protein, 108 aa, synthetic construct [sequence omitted]

SEQ ID NO: 53 - protein, 108 aa, synthetic construct
  REGION 1..108: note = Synthetic polypeptide
DIQLTQSPSFLSASVGDRVTITCRASQSIGRSLHWYQQKPGKAPKLLIKYASQSLSGVPSRFSGSGSGTEFTLTISSLQPEDFATYYCHQSSRLPFTFGPGTKVDIKR

SEQ ID NO: 54 - protein, 107 aa, synthetic construct
  REGION 1..107: note = Synthetic polypeptide
EIVLTQSPATLSLSPGERATLSCRASKSVGRSLHWYQQKPGQAPRLLIKYASQSLSGIPARFSGSGSGTDFTLTISSLEPEDFAVYYCHQSSRLPFTFGPGTKVDIK

SEQ ID NO: 55 - protein, 107 aa, synthetic construct
  REGION 1..107: note = Synthetic polypeptide
EIVLTQSPATLSLSPGERATLSCRASQSVGRSLHWYQQKPGQAPRLLIKYASQSLSGIPARFSGSGSGTDFTLTISSLEPEDFAVYYCHQSSRLPFTFGPGTKVDIK

SEQ ID NO: 56 - protein, 107 aa, synthetic construct
  REGION 1..107: note = Synthetic polypeptide
EIVLTQSPATLSLSPGERATLSCRASQSVWRSLHWYQQKPGQAPRLLIKYASQSLSGIPARFSGSGSGTDFTLTISSLEPEDFAVYYCHQSSRLPFTFGPGTKVDIK

SEQ ID NO: 57 - protein, 107 aa, synthetic construct
  REGION 1..107: note = Synthetic polypeptide
EIVLTQSPATLSLSPGERATLSCRASQSVGRNLHWYQQKPGQAPRLLIKYASQSLSGIPARFSGSGSGTDFTLTISSLEPEDFAVYYCHQSSRLPFTFGPGTKVDIK

SEQ ID NO: 58 - protein, 107 aa, synthetic construct
  REGION 1..107: note = Synthetic polypeptide
EIVLTQSPATLSLSPGERATLSCRASQSVGRSLHWYQQKPGQAPRLLIKYASQSLSGIPARFSGSGSGTDFTLTISSLEPEDFAVYYCHQSSMLPFTFGPGTKVDIK

SEQ ID NO: 59 - protein, 107 aa, synthetic construct
  REGION 1..107: note = Synthetic polypeptide
EIVLTQSPATLSLSPGERATLSCRASKSVWRSLHWYQQKPGQAPRLLIKYASQSLSGIPARFSGSGSGTDFTLTISSLEPEDFAVYYCHQSSRLPFTFGPGTKVDIK

SEQ ID NO: 60 - protein, 107 aa, synthetic construct
  REGION 1..107: note = Synthetic polypeptide
EIVLTQSPATLSLSPGERATLSCRASKSVGRNLHWYQQKPGQAPRLLIKYASQSLSGIPARFSGSGSGTDFTLTISSLEPEDFAVYYCHQSSRLPFTFGPGTKVDIK

SEQ ID NO: 61 - protein, 107 aa, synthetic construct
  REGION 1..107: note = Synthetic polypeptide
EIVLTQSPATLSLSPGERATLSCRASKSVGRSLHWYQQKPGQAPRLLIKYASQSLSGIPARFSGSGSGTDFTLTISSLEPEDFAVYYCHQSSMLPFTFGPGTKVDIK

SEQ ID NO: 62 - protein, 108 aa, synthetic construct
  REGION 1..108: note = Synthetic polypeptide
EIVLTQSPATLSLSPGERATLSCRASKSVGWSLHWYQQKPGQAPRLLIKYASQSLSGIPARFSGSGSGTDFTLTISSLEPEDFAVYYCHQSSRLPFTFGPGTKVDIKR

SEQ ID NO: 63 - protein, 108 aa, synthetic construct
  REGION 1..108: note = Synthetic polypeptide
EIVLTQSPATLSLSPGERATLSCRASKSVGYSLHWYQQKPGQAPRLLIKYASQSLSGIPARFSGSGSGTDFTLTISSLEPEDFAVYYCHQSSRLPFTFGPGTKVDIKR

SEQ ID NO: 64 - protein, 108 aa, synthetic construct
  REGION 1..108: note = Synthetic polypeptide
EIVLTQSPATLSLSPGERATLSCRASKAVGWSLHWYQQKPGQAPRLLIKYASQSLSGIPARFSGSGSGTDFTLTISSLEPEDFAVYYCHQSSRLPFTFGPGTKVDIKR

SEQ ID NO: 65 - protein, 108 aa, synthetic construct
  REGION 1..108: note = Synthetic polypeptide
EIVLTQSPATLSLSPGERATLSCRASKSVGQSLHWYQQKPGQAPRLLIKYASQSLSGIPARFSGSGSGTDFTLTISSLEPEDFAVYYCHQSSRLPFTFGPGTKVDIKR

SEQ ID NO: 66 - protein, 108 aa, synthetic construct
  REGION 1..108: note = Synthetic polypeptide
EIVLTQSPATLSLSPGERATLSCRASRSVGLALHWYQQKPGQAPRLLIKYASQSLSGIPARFSGSGSGTDFTLTISSLEPEDFAVYYCHQSSFLPFTFGPGTKVDIKR

SEQ ID NO: 67 - protein, 108 aa, synthetic construct
  REGION 1..108: note = Synthetic polypeptide
DIQMTQSPSSLSASVGDRITITCRASQSISRYLNWYQQKPGKAPKLLIYAASSLQSGIPSRFSGSGSGTDFTLTINSLQPEDFATYFCQQSYSPPFTFGPGTKVDIKR

SEQ ID NO: 68 - protein, 107 aa, synthetic construct
  REGION 1..107: note = Synthetic polypeptide
DIQMTQSPSSLSASVGDRITITCRASQSISRYLNWYQQKPGKAPKLLIFAGQRLQSGIPSRFSGSGSGTDFTLTINSLQPEDFATYFCQQAIGMPYTFGPGTKVDIK

SEQ ID NO: 69 - protein, 107 aa, synthetic construct
  REGION 1..107: note = Synthetic polypeptide
DIQMTQSPSSLSASVGDRITITCRASQYIVRYLNWYQQKPGKAPKLLIYAAHHLQSGIPSRFSGSGSGTDFTLTINSLQPEDFATYFCQQAIQEPYTFGPGTKVDIK

SEQ ID NO: 70 - protein, 107 aa, synthetic construct
  REGION 1..107: note = Synthetic polypeptide
DIQMTQSPSSLSASVGDRITITCRASQTIVRYLNWYQQKPGKAPKLLIFAGQRLQSGIPSRFSGSGSGTDFTLTINSLQPEDFATYFCQQAIINPYTFGPGTKVDIK

SEQ ID NO: 71 - protein, 107 aa, synthetic construct
  REGION 1..107: note = Synthetic polypeptide
DIQMTQSPSSLSASVGDRITITCRASQYIVRYLNWYQQKPGKAPKLLIYAANMLQSGIPSRFSGSGSGTDFTLTINSLQPEDFATYFCQQAINQPYTFGPGTKVDIK

SEQ ID NO: 72 - protein, 107 aa, synthetic construct
  REGION 1..107: note = Synthetic polypeptide
DIQMTQSPSSLSASVGDRITITCRASQKIARYLVWYQQKPGKAPKLLIYAANMLQSGIPSRFSGSGSGTDFTLTINSLQPEDFATYFCQQSIQQPYTFGPGTKVDIK

SEQ ID NO: 73 - protein, 108 aa, synthetic construct
  REGION 1..108: note = Synthetic polypeptide
DIQMTQSPSSLSASVGDRITITCRASQSISRYLNWYQQKPGKAPKLLIFAGQRLQSGIPSRFSGSGSGTDFTLTINSLQPEDFATYFCQQAIQQPYTFGPGTKVDIKR

SEQ ID NO: 74 - protein, 107 aa, synthetic construct
  REGION 1..107: note = Synthetic polypeptide
DIQMTQSPSSLSASVGDRITITCRASQYIVRYLNWYQQKPGKAPKLLIYASYNLQSGIPSRFSGSGSGTDFTLTINSLQPEDFATYFCQQAIMAPYTFGPGTKVDIK

SEQ ID NO: 75 - protein, 107 aa, synthetic construct
  REGION 1..107: note = Synthetic polypeptide
DIQMTQSPSSLSASVGDRITITCRASQPIAQYLNWYQQKPGKAPKLLIYAGRYLQSGIPSRFSGSGSGTDFTLTINSLQPEDFATYFCQQAIQNPYTFGPGTKVDIK

SEQ ID NO: 76 - protein, 107 aa, synthetic construct
  REGION 1..107: note = Synthetic polypeptide
DIQMTQSPSSLSASVGDRITITCRASQSISRYLNWYQQKPGKAPKLLIFAGQRLQSGIPSRFSGSGSGTDFTLTINSLQPEDFATYFCQQAIVQPYTFGPGTKVDIK

SEQ ID NO: 77 - protein, 107 aa, synthetic construct
  REGION 1..107: note = Synthetic polypeptide
DIQMTQSPSSLSASVGDRITITCRASQPISRYLSWYQQKPGKAPKLLIFAGQRLQSGIPSRFSGSGSGTDFTLTINSLQPEDFATYFCQQAISIPYTFGPGTKVDIK

SEQ ID NO: 78 - protein, 107 aa, synthetic construct
  REGION 1..107: note = Synthetic polypeptide
DIQMTQSPSSLSASVGDRITITCRASQQIARYLNWYQQKPGKAPKLLIYASYNLQSGIPSRFSGSGSGTDFTLTINSLQPEDFATYFCQQAIIQPYTFGPGTKVDIK

SEQ ID NO: 79 - protein, 107 aa, synthetic construct
  REGION 1..107: note = Synthetic polypeptide
DIQMTQSPSSLSASVGDRITITCRASQSISRYLNWYQQKPGKAPKLLIYASYNLQSGIPSRFSGSGSGTDFTLTINSLQPEDFATYFCQQAIQNPYTFGPGTKVDIK

SEQ ID NO: 80 - protein, 107 aa, synthetic construct
  REGION 1..107: note = Synthetic polypeptide
DIQMTQSPSSLSASVGDRITITCRASQTIVRYLNWYQQKPGKAPKLLIFAGQRLQSGIPSRFSGSGSGTDFTLTINSLQPEDFATYFCQQSIQTPYTFGPGTKVDIK

SEQ ID NO: 81 - protein, 107 aa, synthetic construct
  REGION 1..107: note = Synthetic polypeptide
DIQMTQSPSSLSASVGDRITITCRASQSISRYLNWYQQKPGKAPKLLIYAGRILQSGIPSRFSGSGSGTDFTLTINSLQPEDFATYFCQQAIINPYTFGPGTKVDIK

SEQ ID NO: 82 - protein, 107 aa, synthetic construct
  REGION 1..107: note = Synthetic polypeptide
DIQMTQSPSSLSASVGDRITITCRASQRIARYLNWYQQKPGKAPKLLIFAGSILQSGIPSRFSGSGSGTDFTLTINSLQPEDFATYFCQQAIQNPYTFGPGTKVDIK

SEQ ID NO: 83 - protein, 107 aa, synthetic construct
  REGION 1..107: note = Synthetic polypeptide
DIQMTQSPSSLSASVGDRITITCRASQSISRYLNWYQQKPGKAPKLLIYASYNLQSGIPSRFSGSGSGTDFTLTINSLQPEDFATYFCQQSIQQPYTFGPGTKVDIK

SEQ ID NO: 84 - protein, 107 aa, synthetic construct
  REGION 1..107: note = Synthetic polypeptide
DIQMTQSPSSLSASVGDRITITCRASQYIVRYLNWYQQKPGKAPKLLIYASYNLQSGIPSRFSGSGSGTDFTLTINSLQPEDFATYFCQQAIQQPYTFGPGTKVDIK

SEQ ID NO: 85 - protein, 107 aa, synthetic construct
  REGION 1..107: note = Synthetic polypeptide
DIQMTQSPSSLSASVGDRITITCRASQMIARYLNWYQQKPGKAPKLLIYASYNLQSGIPSRFSGSGSGTDFTLTINSLQPEDFATYFCQQAIINPYTFGPGTKVDIK

SEQ ID NO: 86 - protein, 107 aa, synthetic construct
  REGION 1..107: note = Synthetic polypeptide
DIQMTQSPSSLSASVGDRITITCRASQYIVRYLNWYQQKPGKAPKLLIYGARNLQSGIPSRFSGSGSGTDFTLTINSLQPEDFATYFCQQSIQTPYTFGPGTKVDIK

SEQ ID NO: 87 - protein, 107 aa, synthetic construct
  REGION 1..107: note = Synthetic polypeptide
DIQMTQSPSSLSASVGDRITITCRASQSISRYLNWYQQKPGKAPKLLIYAASSLQSGIPSRFSGSGSGTDFTLTINSLQPEDFATYFCQQSYSPPFTFGPGTKVDIK

SEQ ID NO: 88 - protein, 5 aa, synthetic construct
  REGION 1..5: note = Synthetic polypeptide
RFAMH

SEQ ID NO: 89 - protein, 5 aa, synthetic construct
  REGION 1..5: note = Synthetic polypeptide
HFAMH

SEQ ID NO: 90 - protein, 5 aa, synthetic construct
  REGION 1..5: note = Synthetic polypeptide
RYAMH

SEQ ID NO: 91 - protein, 5 aa, synthetic construct
  REGION 1..5: note = Synthetic polypeptide
KYAMH

SEQ ID NO: 92 - protein, 5 aa, synthetic construct
  REGION 1..5: note = Synthetic polypeptide
KFAMH

SEQ ID NO: 93 - protein, 5 aa, synthetic construct
  REGION 1..5: note = Synthetic polypeptide
HYAMH

SEQ ID NO: 94 - protein, 5 aa, synthetic construct
  REGION 1..5: note = Synthetic polypeptide
YFAMH

SEQ ID NO: 95 - protein, 5 aa, synthetic construct
  REGION 1..5: note = Synthetic polypeptide
FYAMH

SEQ ID NO: 96 - protein, 5 aa, synthetic construct
  REGION 1..5: note = Synthetic polypeptide
YYAMH

SEQ ID NO: 97 - protein, 7 aa, synthetic construct
  REGION 1..7: note = Synthetic polypeptide
SNSATWN

SEQ ID NO: 98 - protein, 7 aa, synthetic construct
  REGION 1..7: note = Synthetic polypeptide
NRLATWN

SEQ ID NO: 99 - protein, 7 aa, synthetic construct
  REGION 1..7: note = Synthetic polypeptide
SRQATWN

SEQ ID NO: 100 - protein, 7 aa, synthetic construct
  REGION 1..7: note = Synthetic polypeptide
NKQATWN

SEQ ID NO: 101 - protein, 7 aa, synthetic construct
  REGION 1..7: note = Synthetic polypeptide
SNHATWN

SEQ ID NO: 102 - protein, 7 aa, synthetic construct
  REGION 1..7: note = Synthetic polypeptide
SRYATWN

SEQ ID NO: 103 - protein, 7 aa, synthetic construct
  REGION 1..7: note = Synthetic polypeptide
SHVATWN

SEQ ID NO: 104 - protein, 7 aa, synthetic construct
  REGION 1..7: note = Synthetic polypeptide
NHQATWN

SEQ ID NO: 105 - protein, 7 aa, synthetic construct
  REGION 1..7: note = Synthetic polypeptide
SRDATWN

SEQ ID NO: 106 - protein, 17 aa, synthetic construct
  REGION 1..17: note = Synthetic polypeptide
VISYDGGNKYYAESVKG

SEQ ID NO: 107 - protein, 17 aa, synthetic construct
  REGION 1..17: note = Synthetic polypeptide
VISYNGGNKYYAESVKG

SEQ ID NO: 108 - protein, 17 aa, synthetic construct
  REGION 1..17: note = Synthetic polypeptide
VISYIGGNKYYAESVKG

SEQ ID NO: 109 - protein, 17 aa, synthetic construct
  REGION 1..17: note = Synthetic polypeptide
VISYQGGNKYYAESVKG

SEQ ID NO: 110 - protein, 17 aa, synthetic construct
  REGION 1..17: note = Synthetic polypeptide
VISYYGGNKYYAESVKG

SEQ ID NO: 111 - protein, 17 aa, synthetic construct
  REGION 1..17: note = Synthetic polypeptide
VISYDGRNKYYAESVKG

SEQ ID NO: 112 - protein, 17 aa, synthetic construct
  REGION 1..17: note = Synthetic polypeptide
VISYDGNNKYYAESVKG

SEQ ID NO: 113 - protein, 17 aa, synthetic construct
  REGION 1..17: note = Synthetic polypeptide
VISYDGGNKYYARSVKG

SEQ ID NO: 114 - protein, 17 aa, synthetic construct
  REGION 1..17: note = Synthetic polypeptide
VISYNGRNKYYAESVKG

SEQ ID NO: 115 - protein, 17 aa, synthetic construct
  REGION 1..17: note = Synthetic polypeptide
VISYIGRNKYYAESVKG

SEQ ID NO: 116 - protein, 17 aa, synthetic construct
  REGION 1..17: note = Synthetic polypeptide
VISYQGRNKYYAESVKG

SEQ ID NO: 117 - protein, 17 aa, synthetic construct
  REGION 1..17: note = Synthetic polypeptide
VISYYGRNKYYAESVKG

SEQ ID NO: 118 - protein, 17 aa, synthetic construct
  REGION 1..17: note = Synthetic polypeptide
VISYNGNNKYYAESVKG

SEQ ID NO: 119 - protein, 17 aa, synthetic construct
  REGION 1..17: note = Synthetic polypeptide
VISYIGNNKYYAESVKG

SEQ ID NO: 120 - protein, 17 aa, synthetic construct
  REGION 1..17: note = Synthetic polypeptide
VISYQGNNKYYAESVKG

SEQ ID NO: 121 - protein, 17 aa, synthetic construct
  REGION 1..17: note = Synthetic polypeptide
VISYYGNNKYYAESVKG

SEQ ID NO: 122 - protein, 17 aa, synthetic construct
  REGION 1..17: note = Synthetic polypeptide
VISYNGGNKYYARSVKG

SEQ ID NO: 123 - protein, 17 aa, synthetic construct
  REGION 1..17: note = Synthetic polypeptide
VISYIGGNKYYARSVKG

SEQ ID NO: 124 - protein, 17 aa, synthetic construct
  REGION 1..17: note = Synthetic polypeptide
VISYQGGNKYYARSVKG

SEQ ID NO: 125 - protein, 17 aa, synthetic construct
  REGION 1..17: note = Synthetic polypeptide
VISYYGGNKYYARSVKG

SEQ ID NO: 126 - protein, 17 aa, synthetic construct
  REGION 1..17: note = Synthetic polypeptide
VISYDGRNKYYARSVKG

SEQ ID NO: 127 - protein, 17 aa, synthetic construct
  REGION 1..17: note = Synthetic polypeptide
VISYDGNNKYYARSVKG

SEQ ID NO: 128 - protein, 17 aa, synthetic construct
  REGION 1..17: note = Synthetic polypeptide
VISYNGRNKYYARSVKG

SEQ ID NO: 129 - protein, 17 aa, synthetic construct
  REGION 1..17: note = Synthetic polypeptide
VISYIGRNKYYARSVKG

SEQ ID NO: 130 - protein, 17 aa, synthetic construct
  REGION 1..17: note = Synthetic polypeptide
VISYQGRNKYYARSVKG

SEQ ID NO: 131 - protein, 17 aa, synthetic construct
  REGION 1..17: note = Synthetic polypeptide
VISYYGRNKYYARSVKG

SEQ ID NO: 132 - protein, 17 aa, synthetic construct
  REGION 1..17: note = Synthetic polypeptide
VISYNGNNKYYARSVKG

SEQ ID NO: 133 - protein, 17 aa, synthetic construct
  REGION 1..17: note = Synthetic polypeptide
VISYIGNNKYYARSVKG

SEQ ID NO: 134 - protein, 17 aa, synthetic construct
  REGION 1..17: note = Synthetic polypeptide
VISYQGNNKYYARSVKG

SEQ ID NO: 135 - protein, 17 aa, synthetic construct
  REGION 1..17: note = Synthetic polypeptide
VISYYGNNKYYARSVKG

SEQ ID NO: 136 - protein, 17 aa, synthetic construct
  REGION 1..17: note = Synthetic polypeptide
VISFDGGNKYYAESVKG

SEQ ID NO: 137 - protein, 17 aa, synthetic construct
  REGION 1..17: note = Synthetic polypeptide
VISYDGANKYYAESVKG

SEQ ID NO: 138 - protein, 17 aa, synthetic construct
  REGION 1..17: note = Synthetic polypeptide
VISFNGGNKYYAESVKG

SEQ ID NO: 139 - protein, 17 aa, synthetic construct
  REGION 1..17: note = Synthetic polypeptide
VISYNGANKYYAESVKG

SEQ ID NO: 140 - protein, 17 aa, synthetic construct
  REGION 1..17: note = Synthetic polypeptide
VISFDGRNKYYAESVKG

SEQ ID NO: 141 - protein, 17 aa, synthetic construct
  REGION 1..17: note = Synthetic polypeptide
VISFDGGNKYYARSVKG

SEQ ID NO: 142 - protein, 17 aa, synthetic construct
  REGION 1..17: note = Synthetic polypeptide
VISYDGANKYYARSVKG

SEQ ID NO: 143 - protein, 17 aa, synthetic construct
  REGION 1..17: note = Synthetic polypeptide
VISFKGSNKYYAESVKG

SEQ ID NO: 144 - protein, 17 aa, synthetic construct
  REGION 1..17: note = Synthetic polypeptide
VISFSGSNKYYAESVKG

SEQ ID NO: 145 - protein, 17 aa, synthetic construct
  REGION 1..17: note = Synthetic polypeptide
VISYRGGNKYYAESVKG

SEQ ID NO: 146 - protein, 17 aa, synthetic construct
  REGION 1..17: note = Synthetic polypeptide
VISYSGANKYYAESVKG

SEQ ID NO: 147 - protein, 17 aa, synthetic construct
  REGION 1..17: note = Synthetic polypeptide
VISFKGANKYYAESVKG

SEQ ID NO: 148 - protein, 17 aa, synthetic construct
  REGION 1..17: note = Synthetic polypeptide
VISYRGANKYYAESVKG

SEQ ID NO: 149 - protein, 17 aa, synthetic construct
  REGION 1..17: note = Synthetic polypeptide
VISFYGSNKYYAESVKG

SEQ ID NO: 150 - protein, 17 aa, synthetic construct
  REGION 1..17: note = Synthetic polypeptide
VISFFGGNKYYAESVKG

SEQ ID NO: 151 - protein, 17 aa, synthetic construct
  REGION 1..17: note = Synthetic polypeptide
VISFMGTNKYYAESVKG

SEQ ID NO: 152 - protein, 17 aa, synthetic construct
  REGION 1..17: note = Synthetic polypeptide
VISHRGTNKYYAESVKG

SEQ ID NO: 153 - protein, 17 aa, synthetic construct
  REGION 1..17: note = Synthetic polypeptide
VINYRGHGKYYAESVKG

SEQ ID NO: 154 - protein, 17 aa, synthetic construct
  REGION 1..17: note = Synthetic polypeptide
VISFSGGSKYYAESVKG

SEQ ID NO: 155 - protein, 17 aa, synthetic construct
  REGION 1..17: note = Synthetic polypeptide
VISYTGQFKYYAESVKG

SEQ ID NO: 156 - protein, 17 aa, synthetic construct
  REGION 1..17: note = Synthetic polypeptide
VISYTGAQKYYAESVKG

SEQ ID NO: 157 - protein, 17 aa, synthetic construct
  REGION 1..17: note = Synthetic polypeptide
VISYSGASKYYAESVKG

SEQ ID NO: 158 - protein, 17 aa, synthetic construct
  REGION 1..17: note = Synthetic polypeptide
VISYSGAFKYYAESVKG

SEQ ID NO: 159 - protein, 17 aa, synthetic construct
  REGION 1..17: note = Synthetic polypeptide
VITYTGGAKYYAESVKG

SEQ ID NO: 160 - protein, 17 aa, synthetic construct
  REGION 1..17: note = Synthetic polypeptide
VINFQGTTKYYAESVKG

SEQ ID NO: 161 - protein, 17 aa, synthetic construct
  REGION 1..17: note = Synthetic polypeptide
VISYSGDLKYYAESVKG

SEQ ID NO: 162 - protein, 17 aa, synthetic construct
  REGION 1..17: note = Synthetic polypeptide
VINYFGDAKYYAESVKG

SEQ ID NO: 163 - protein, 17 aa, synthetic construct
  REGION 1..17: note = Synthetic polypeptide
VISSFGSNKYYAESVKG

SEQ ID NO: 164 - protein, 17 aa, synthetic construct
  REGION 1..17: note = Synthetic polypeptide
VISYSGSNKYYAESVKG

SEQ ID NO: 165 - protein, 17 aa, synthetic construct
  REGION 1..17: note = Synthetic polypeptide
VISHYGTNKYYAESVKG

SEQ ID NO: 166 - protein, 17 aa, synthetic construct
  REGION 1..17: note = Synthetic polypeptide
VISYQGSNKYYAESVKG

SEQ ID NO: 167 - protein, 18 aa, synthetic construct
  REGION 1..18: note = Synthetic polypeptide
RTYYRSKWSNHYAVSVKS

SEQ ID NO: 168 - protein, 18 aa, synthetic construct
  REGION 1..18: note = Synthetic polypeptide
RTYYRGKWKNHYAVSVKS

SEQ ID NO: 169 - protein, 18 aa, synthetic construct
  REGION 1..18: note = Synthetic polypeptide
RTYYRGQWKNHYAVSVKS

SEQ ID NO: 170 - protein, 18 aa, synthetic construct
  REGION 1..18: note = Synthetic polypeptide
RTYFRRTWKNHYAVSVKS

SEQ ID NO: 171 - protein, 11 aa, synthetic construct
  REGION 1..11: note = Synthetic polypeptide
GYDVLTGYPDY

SEQ ID NO: 172 - protein, 11 aa, synthetic construct
  REGION 1..11: note = Synthetic polypeptide
GYDILTGYPDY

SEQ ID NO: 173 - protein, 11 aa, synthetic construct
  REGION 1..11: note = Synthetic polypeptide
GYDFLTGYPDY

SEQ ID NO: 174 - protein, 11 aa, synthetic construct
  REGION 1..11: note = Synthetic polypeptide
GYDLLTGYPDY

SEQ ID NO: 175 - protein, 11 aa, synthetic construct
  REGION 1..11: note = Synthetic polypeptide
GYDMLTGYPDY

SEQ ID NO: 176 - protein, 11 aa, synthetic construct
  REGION 1..11: note = Synthetic polypeptide
GYDLLSGYPDY

SEQ ID NO: 177 - protein, 11 aa, synthetic construct
  REGION 1..11: note = Synthetic polypeptide
GYDPLTGYPDY

SEQ ID NO: 178 - protein, 11 aa, synthetic construct
  REGION 1..11: note = Synthetic polypeptide
GTWKQLWFLDH

SEQ ID NO: 179 - protein, 11 aa, synthetic construct
  REGION 1..11: note = Synthetic polypeptide
GTWNQDWFLDH

SEQ ID NO: 180 - protein, 11 aa, synthetic construct
  REGION 1..11: note = Synthetic polypeptide
GTWDQDWFLDH

SEQ ID NO: 181 - protein, 11 aa, synthetic construct
  REGION 1..11: note = Synthetic polypeptide
GTWEQDWFLDH

SEQ ID NO: 182 - protein, 11 aa, synthetic construct
  REGION 1..11: note = Synthetic polypeptide
GMWNQNWFLDH

SEQ ID NO: 183 - protein, 11 aa, synthetic construct
  REGION 1..11: note = Synthetic polypeptide
GTWIGDWFMDH

SEQ ID NO: 184 - protein, 11 aa, synthetic construct
  REGION 1..11: note = Synthetic polypeptide
GTWIQDWFLDH

SEQ ID NO: 185 - protein, 11 aa, synthetic construct
  REGION 1..11: note = Synthetic polypeptide
GMWSGDWFLDH

SEQ ID NO: 186 - protein, 11 aa, synthetic construct
  REGION 1..11: note = Synthetic polypeptide
GMWSEDWFLDH

SEQ ID NO: 187 - protein, 11 aa, synthetic construct
  REGION 1..11: note = Synthetic polypeptide
GMWQGNWFLDH

SEQ ID NO: 188 - protein, 11 aa, synthetic construct
  REGION 1..11: note = Synthetic polypeptide
GQWNEDWFLDH

SEQ ID NO: 189 - protein, 11 aa, synthetic construct
  REGION 1..11: note = Synthetic polypeptide
GRWEGDWFFDH

SEQ ID NO: 190 - protein, 11 aa, synthetic construct
  REGION 1..11: note = Synthetic polypeptide
GVWIGNWFLDH

SEQ ID NO: 191 - protein, 120 aa, synthetic construct [sequence omitted]

SEQ ID NO: 192 - protein, 120 aa, synthetic construct
  REGION 1..120: note = Synthetic polypeptide
QVQLVESGAEVVKPGASVKVSCKASGFTFSRFAMHWVRQAPGQGLEWMGVISYDGGNKYYAESVKGRVTMTRDTSTSTLYMELSSLRSEDTAVYYCARGYDVLTGYPDYWGQGTLVTVSS

SEQ ID NO: 193 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYNGGNKYYAESVKGRFTISRDNSKNTLYLQMNSLRAEDTALFYCARGYDVLTGYPDYWGQGTLVTVSS

SEQ ID NO: 194 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYIGGNKYYAESVKGRFTISRDNSKNTLYLQMNSLRAEDTALFYCARGYDVLTGYPDYWGQGTLVTVSS

SEQ ID NO: 195 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYQGGNKYYAESVKGRFTISRDNSKNTLYLQMNSLRAEDTALFYCARGYDVLTGYPDYWGQGTLVTVSS

SEQ ID NO: 196 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYYGGNKYYAESVKGRFTISRDNSKNTLYLQMNSLRAEDTALFYCARGYDVLTGYPDYWGQGTLVTVSS

SEQ ID NO: 197 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYDGRNKYYAESVKGRFTISRDNSKNTLYLQMNSLRAEDTALFYCARGYDVLTGYPDYWGQGTLVTVSS

SEQ ID NO: 198 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYDGNNKYYAESVKGRFTISRDNSKNTLYLQMNSLRAEDTALFYCARGYDVLTGYPDYWGQGTLVTVSS

SEQ ID NO: 199 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYDGGNKYYARSVKGRFTISRDNSKNTLYLQMNSLRAEDTALFYCARGYDVLTGYPDYWGQGTLVTVSS

SEQ ID NO: 200 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYDGGNKYYAESVKGRFTISRDNSKNTLYLQMNSLRAEDTALFYCARGYDILTGYPDYWGQGTLVTVSS

SEQ ID NO: 201 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYNGRNKYYAESVKGRFTISRDNSKNTLYLQMNSLRAEDTALFYCARGYDVLTGYPDYWGQGTLVTVSS

SEQ ID NO: 202 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYIGRNKYYAESVKGRFTISRDNSKNTLYLQMNSLRAEDTALFYCARGYDVLTGYPDYWGQGTLVTVSS

SEQ ID NO: 203 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYQGRNKYYAESVKGRFTISRDNSKNTLYLQMNSLRAEDTALFYCARGYDVLTGYPDYWGQGTLVTVSS

SEQ ID NO: 204 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYYGRNKYYAESVKGRFTISRDNSKNTLYLQMNSLRAEDTALFYCARGYDVLTGYPDYWGQGTLVTVSS

SEQ ID NO: 205 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYNGNNKYYAESVKGRFTISRDNSKNTLYLQMNSLRAEDTALFYCARGYDVLTGYPDYWGQGTLVTVSS

SEQ ID NO: 206 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYIGNNKYYAESVKGRFTISRDNSKNTLYLQMNSLRAEDTALFYCARGYDVLTGYPDYWGQGTLVTVSS

SEQ ID NO: 207 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYQGNNKYYAESVKGRFTISRDNSKNTLYLQMNSLRAEDTALFYCARGYDVLTGYPDYWGQGTLVTVSS

SEQ ID NO: 208 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYYGNNKYYAESVKGRFTISRDNSKNTLYLQMNSLRAEDTALFYCARGYDVLTGYPDYWGQGTLVTVSS

SEQ ID NO: 209 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYNGGNKYYARSVKGRFTISRDNSKNTLYLQMNSLRAEDTALFYCARGYDVLTGYPDYWGQGTLVTVSS

SEQ ID NO: 210 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYIGGNKYYARSVKGRFTISRDNSKNTLYLQMNSLRAEDTALFYCARGYDVLTGYPDYWGQGTLVTVSS

SEQ ID NO: 211 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYQGGNKYYARSVKGRFTISRDNSKNTLYLQMNSLRAEDTALFYCARGYDVLTGYPDYWGQGTLVTVSS

SEQ ID NO: 212 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYYGGNKYYARSVKGRFTISRDNSKNTLYLQMNSLRAEDTALFYCARGYDVLTGYPDYWGQGTLVTVSS

SEQ ID NO: 213 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYNGGNKYYAESVKGRFTISRDNSKNTLYLQMNSLRAEDTALFYCARGYDILTGYPDYWGQGTLVTVSS

SEQ ID NO: 214 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYIGGNKYYAESVKGRFTISRDNSKNTLYLQMNSLRAEDTALFYCARGYDILTGYPDYWGQGTLVTVSS

SEQ ID NO: 215 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYQGGNKYYAESVKGRFTISRDNSKNTLYLQMNSLRAEDTALFYCARGYDILTGYPDYWGQGTLVTVSS

SEQ ID NO: 216 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYYGGNKYYAESVKGRFTISRDNSKNTLYLQMNSLRAEDTALFYCARGYDILTGYPDYWGQGTLVTVSS

SEQ ID NO: 217 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYDGRNKYYARSVKGRFTISRDNSKNTLYLQMNSLRAEDTALFYCARGYDVLTGYPDYWGQGTLVTVSS

SEQ ID NO: 218 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYDGNNKYYARSVKGRFTISRDNSKNTLYLQMNSLRAEDTALFYCARGYDVLTGYPDYWGQGTLVTVSS

SEQ ID NO: 219 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYDGRNKYYAESVKGRFTISRDNSKNTLYLQMNSLRAEDTALFYCARGYDILTGYPDYWGQGTLVTVSS

SEQ ID NO: 220 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYDGNNKYYAESVKGRFTISRDNSKNTLYLQMNSLRAEDTALFYCARGYDILTGYPDYWGQGTLVTVSS

SEQ ID NO: 221 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYDGGNKYYARSVKGRFTISRDNSKNTLYLQMNSLRAEDTALFYCARGYDILTGYPDYWGQGTLVTVSS

SEQ ID NO: 222 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYNGRNKYYARSVKGRFTISRDNSKNTLYLQMNSLRAEDTALFYCARGYDVLTGYPDYWGQGTLVTVSS

SEQ ID NO: 223 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYIGRNKYYARSVKGRFTISRDNSKNTLYLQMNSLRAEDTALFYCARGYDVLTGYPDYWGQGTLVTVSS

SEQ ID NO: 224 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYQGRNKYYARSVKGRFTISRDNSKNTLYLQMNSLRAEDTALFYCARGYDVLTGYPDYWGQGTLVTVSS

SEQ ID NO: 225 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYYGRNKYYARSVKGRFTISRDNSKNTLYLQMNSLRAEDTALFYCARGYDVLTGYPDYWGQGTLVTVSS

SEQ ID NO: 226 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYNGNNKYYARSVKGRFTISRDNSKNTLYLQMNSLRAEDTALFYCARGYDVLTGYPDYWGQGTLVTVSS

SEQ ID NO: 227 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYIGNNKYYARSVKGRFTISRDNSKNTLYLQMNSLRAEDTALFYCARGYDVLTGYPDYWGQGTLVTVSS

SEQ ID NO: 228 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYQGNNKYYARSVKGRFTISRDNSKNTLYLQMNSLRAEDTALFYCARGYDVLTGYPDYWGQGTLVTVSS

SEQ ID NO: 229 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYYGNNKYYARSVKGRFTISRDNSKNTLYLQMNSLRAEDTALFYCARGYDVLTGYPDYWGQGTLVTVSS

SEQ ID NO: 230 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYNGRNKYYAESVKGRFTISRDNSKNTLYLQMNSLRAEDTALFYCARGYDILTGYPDYWGQGTLVTVSS

SEQ ID NO: 231 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYIGRNKYYAESVKGRFTISRDNSKNTLYLQMNSLRAEDTALFYCARGYDILTGYPDYWGQGTLVTVSS

SEQ ID NO: 232 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYQGRNKYYAESVKGRFTISRDNSKNTLYLQMNSLRAEDTALFYCARGYDILTGYPDYWGQGTLVTVSS

SEQ ID NO: 233 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYYGRNKYYAESVKGRFTISRDNSKNTLYLQMNSLRAEDTALFYCARGYDILTGYPDYWGQGTLVTVSS

SEQ ID NO: 234 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYNGNNKYYAESVKGRFTISRDNSKNTLYLQMNSLRAEDTALFYCARGYDILTGYPDYWGQGTLVTVSS

SEQ ID NO: 235 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYIGNNKYYAESVKGRFTISRDNSKNTLYLQMNSLRAEDTALFYCARGYDILTGYPDYWGQGTLVTVSS

SEQ ID NO: 236 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYQGNNKYYAESVKGRFTISRDNSKNTLYLQMNSLRAEDTALFYCARGYDILTGYPDYWGQGTLVTVSS

SEQ ID NO: 237 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYYGNNKYYAESVKGRFTISRDNSKNTLYLQMNSLRAEDTALFYCARGYDILTGYPDYWGQGTLVTVSS

SEQ ID NO: 238 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYDGRNKYYARSVKGRFTISRDNSKNTLYLQMNSLRAEDTALFYCARGYDILTGYPDYWGQGTLVTVSS

SEQ ID NO: 239 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYDGNNKYYARSVKGRFTISRDNSKNTLYLQMNSLRAEDTALFYCARGYDILTGYPDYWGQGTLVTVSS

SEQ ID NO: 240 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYNGRNKYYARSVKGRFTISRDNSKNTLYLQMNSLRAEDTALFYCARGYDILTGYPDYWGQGTLVTVSS

SEQ ID NO: 241 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYIGRNKYYARSVKGRFTISRDNSKNTLYLQMNSLRAEDTALFYCARGYDILTGYPDYWGQGTLVTVSS

SEQ ID NO: 242 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYQGRNKYYARSVKGRFTISRDNSKNTLYLQMNSLRAEDTALFYCARGYDILTGYPDYWGQGTLVTVSS

SEQ ID NO: 243 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYYGRNKYYARSVKGRFTISRDNSKNTLYLQMNSLRAEDTALFYCARGYDILTGYPDYWGQGTLVTVSS

SEQ ID NO: 244 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYNGNNKYYARSVKGRFTISRDNSKNTLYLQMNSLRAEDTALFYCARGYDILTGYPDYWGQGTLVTVSS

SEQ ID NO: 245 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYIGNNKYYARSVKGRFTISRDNSKNTLYLQMNSLRAEDTALFYCARGYDILTGYPDYWGQGTLVTVSS

SEQ ID NO: 246 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYQGNNKYYARSVKGRFTISRDNSKNTLYLQMNSLRAEDTALFYCARGYDILTGYPDYWGQGTLVTVSS

SEQ ID NO: 247 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYYGNNKYYARSVKGRFTISRDNSKNTLYLQMNSLRAEDTALFYCARGYDILTGYPDYWGQGTLVTVSS

SEQ ID NO: 248 - protein, 120 aa, synthetic construct
  REGION 1..120: note = Synthetic polypeptide
QVQLVESGGGVVQPGRSLRLSCAASGFTFSHFAMHWVRQAPGKGLEWVAVISYDGGNKYYAESVKGRFTISRDNSKNTLYLQMNSLRAEDTALFYCARGYDVLTGYPDYWGQGTLVTVSS

SEQ ID NO: 249 - protein, 120 aa, synthetic construct
  REGION 1..120: note = Synthetic polypeptide
QVQLVESGGGVVQPGRSLRLSCAASGFTFSRYAMHWVRQAPGKGLEWVAVISYDGGNKYYAESVKGRFTISRDNSKNTLYLQMNSLRAEDTALFYCARGYDVLTGYPDYWGQGTLVTVSS

SEQ ID NO: 250 - protein, 120 aa, synthetic construct
  REGION 1..120: note = Synthetic polypeptide
QVQLVESGGGVVQPGRSLRLSCAASGFTFSRFAMHWVRQAPGKGLEWVAVISFDGGNKYYAESVKGRFTISRDNSKNTLYLQMNSLRAEDTALFYCARGYDVLTGYPDYWGQGTLVTVSS

SEQ ID NO: 251 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYDGANKYYAESVKGRFTISRDNSKNTLYLQMNSLRAEDTALFYCARGYDVLTGYPDYWGQGTLVTVSS

SEQ ID NO: 252 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYDGGNKYYAESVKGRFTISRDNSKNTLYLQMNSLRAEDTALFYCARGYDFLTGYPDYWGQGTLVTVSS

SEQ ID NO: 253 - protein, 120 aa, synthetic construct
  REGION 1..120: note = Synthetic polypeptide
QVQLVESGGGVVQPGRSLRLSCAASGFTFSHFAMHWVRQAPGKGLEWVAVISYNGGNKYYAESVKGRFTISRDNSKNTLYLQMNSLRAEDTALFYCARGYDVLTGYPDYWGQGTLVTVSS

SEQ ID NO: 254 - protein, 120 aa, synthetic construct
  REGION 1..120: note = Synthetic polypeptide
QVQLVESGGGVVQPGRSLRLSCAASGFTFSRYAMHWVRQAPGKGLEWVAVISYNGGNKYYAESVKGRFTISRDNSKNTLYLQMNSLRAEDTALFYCARGYDVLTGYPDYWGQGTLVTVSS

SEQ ID NO: 255 - protein, 120 aa, synthetic construct
  REGION 1..120: note = Synthetic polypeptide
QVQLVESGGGVVQPGRSLRLSCAASGFTFSRFAMHWVRQAPGKGLEWVAVISFNGGNKYYAESVKGRFTISRDNSKNTLYLQMNSLRAEDTALFYCARGYDVLTGYPDYWGQGTLVTVSS

SEQ ID NO: 256 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYNGANKYYAESVKGRFTISRDNSKNTLYLQMNSLRAEDTALFYCARGYDVLTGYPDYWGQGTLVTVSS

SEQ ID NO: 257 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYNGGNKYYAESVKGRFTISRDNSKNTLYLQMNSLRAEDTALFYCARGYDFLTGYPDYWGQGTLVTVSS

SEQ ID NO: 258 - protein, 120 aa, synthetic construct
  REGION 1..120: note = Synthetic polypeptide
QVQLVESGGGVVQPGRSLRLSCAASGFTFSHFAMHWVRQAPGKGLEWVAVISYDGRNKYYAESVKGRFTISRDNSKNTLYLQMNSLRAEDTALFYCARGYDVLTGYPDYWGQGTLVTVSS

SEQ ID NO: 259 - protein, 120 aa, synthetic construct
  REGION 1..120: note = Synthetic polypeptide
QVQLVESGGGVVQPGRSLRLSCAASGFTFSRYAMHWVRQAPGKGLEWVAVISYDGRNKYYAESVKGRFTISRDNSKNTLYLQMNSLRAEDTALFYCARGYDVLTGYPDYWGQGTLVTVSS

SEQ ID NO: 260 - protein, 120 aa, synthetic construct
  REGION 1..120: note = Synthetic polypeptide
QVQLVESGGGVVQPGRSLRLSCAASGFTFSRFAMHWVRQAPGKGLEWVAVISFDGRNKYYAESVKGRFTISRDNSKNTLYLQMNSLRAEDTALFYCARGYDVLTGYPDYWGQGTLVTVSS

SEQ ID NO: 261 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYDGRNKYYAESVKGRFTISRDNSKNTLYLQMNSLRAEDTALFYCARGYDFLTGYPDYWGQGTLVTVSS

SEQ ID NO: 262 - protein, 120 aa, synthetic construct
  REGION 1..120: note = Synthetic polypeptide
QVQLVESGGGVVQPGRSLRLSCAASGFTFSHFAMHWVRQAPGKGLEWVAVISYDGGNKYYARSVKGRFTISRDNSKNTLYLQMNSLRAEDTALFYCARGYDVLTGYPDYWGQGTLVTVSS

SEQ ID NO: 263 - protein, 120 aa, synthetic construct
  REGION 1..120: note = Synthetic polypeptide
QVQLVESGGGVVQPGRSLRLSCAASGFTFSRYAMHWVRQAPGKGLEWVAVISYDGGNKYYARSVKGRFTISRDNSKNTLYLQMNSLRAEDTALFYCARGYDVLTGYPDYWGQGTLVTVSS

SEQ ID NO: 264 - protein, 120 aa, synthetic construct
  REGION 1..120: note = Synthetic polypeptide
QVQLVESGGGVVQPGRSLRLSCAASGFTFSRFAMHWVRQAPGKGLEWVAVISFDGGNKYYARSVKGRFTISRDNSKNTLYLQMNSLRAEDTALFYCARGYDVLTGYPDYWGQGTLVTVSS

SEQ ID NO: 265 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYDGANKYYARSVKGRFTISRDNSKNTLYLQMNSLRAEDTALFYCARGYDVLTGYPDYWGQGTLVTVSS

SEQ ID NO: 266 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYDGGNKYYARSVKGRFTISRDNSKNTLYLQMNSLRAEDTALFYCARGYDFLTGYPDYWGQGTLVTVSS

SEQ ID NO: 267 - protein, 120 aa, synthetic construct
  REGION 1..120: note = Synthetic polypeptide
QVQLVESGGGVVQPGRSLRLSCAASGFTFSHFAMHWVRQAPGKGLEWVAVISYDGGNKYYAESVKGRFTISRDNSKNTLYLQMNSLRAEDTALFYCARGYDILTGYPDYWGQGTLVTVSS

SEQ ID NO: 268 - protein, 120 aa, synthetic construct
  REGION 1..120: note = Synthetic polypeptide
QVQLVESGGGVVQPGRSLRLSCAASGFTFSRYAMHWVRQAPGKGLEWVAVISYDGGNKYYAESVKGRFTISRDNSKNTLYLQMNSLRAEDTALFYCARGYDILTGYPDYWGQGTLVTVSS

SEQ ID NO: 269 - protein, 120 aa, synthetic construct
  REGION 1..120: note = Synthetic polypeptide
QVQLVESGGGVVQPGRSLRLSCAASGFTFSRFAMHWVRQAPGKGLEWVAVISFDGGNKYYAESVKGRFTISRDNSKNTLYLQMNSLRAEDTALFYCARGYDILTGYPDYWGQGTLVTVSS

SEQ ID NO: 270 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYDGANKYYAESVKGRFTISRDNSKNTLYLQMNSLRAEDTALFYCARGYDILTGYPDYWGQGTLVTVSS

SEQ ID NO: 271 - protein, 120 aa, synthetic construct
  REGION 1..120: note = Synthetic polypeptide
QVQLVESGGGVVQPGRSLRLSCAASGFTFSKYAMHWVRQAPGKGLEWVAVISFKGSNKYYAESVKGRFTISRDNSKNTLYLQMNSLRAEDTALFYCARGYDLLTGYPDYWGQGTLVTVSS

SEQ ID NO: 272 - protein, 120 aa, synthetic construct
  REGION 1..120: note = Synthetic polypeptide
QVQLVESGGGVVQPGRSLRLSCAASGFTFSRYAMHWVRQAPGKGLEWVAVISYQGGNKYYAESVKGRFTISRDNSKNTLYLQMNSLRAEDTALFYCARGYDLLTGYPDYWGQGTLVTVSS

SEQ ID NO: 273 - protein, 120 aa, synthetic construct
  REGION 1..120: note = Synthetic polypeptide
QVQLVESGGGVVQPGRSLRLSCAASGFTFSHFAMHWVRQAPGKGLEWVAVISFSGSNKYYAESVKGRFTISRDNSKNTLYLQMNSLRAEDTALFYCARGYDMLTGYPDYWGQGTLVTVSS

SEQ ID NO: 274 - protein, 120 aa, synthetic construct
  REGION 1..120: note = Synthetic polypeptide
QVQLVESGGGVVQPGRSLRLSCAASGFTFSKFAMHWVRQAPGKGLEWVAVISYRGGNKYYAESVKGRFTISRDNSKNTLYLQMNSLRAEDTALFYCARGYDLLTGYPDYWGQGTLVTVSS

SEQ ID NO: 275 - protein, 120 aa, synthetic construct
  REGION 1..120: note = Synthetic polypeptide
QVQLVESGGGVVQPGRSLRLSCAASGFTFSRYAMHWVRQAPGKGLEWVAVISYSGANKYYAESVKGRFTISRDNSKNTLYLQMNSLRAEDTALFYCARGYDLLSGYPDYWGQGTLVTVSS

SEQ ID NO: 276 - protein, 120 aa, synthetic construct
  REGION 1..120: note = Synthetic polypeptide
QVQLVESGGGVVQPGRSLRLSCAASGFTFSHYAMHWVRQAPGKGLEWVAVISFKGANKYYAESVKGRFTISRDNSKNTLYLQMNSLRAEDTALFYCARGYDLLTGYPDYWGQGTLVTVSS

SEQ ID NO: 277 - protein, 120 aa, synthetic construct
  REGION 1..120: note = Synthetic polypeptide
QVQLVESGGGVVQPGRSLRLSCAASGFTFSHYAMHWVRQAPGKGLEWVAVISYRGANKYYAESVKGRFTISRDNSKNTLYLQMNSLRAEDTALFYCARGYDLLTGYPDYWGQGTLVTVSS

SEQ ID NO: 278 - protein, 120 aa, synthetic construct
  REGION 1..120: note = Synthetic polypeptide
QVQLVESGGGVVQPGRSLRLSCAASGFTFSHYAMHWVRQAPGKGLEWVAVISFYGSNKYYAESVKGRFTISRDNSKNTLYLQMNSLRAEDTALFYCARGYDFLTGYPDYWGQGTLVTVSS

SEQ ID NO: 279 - protein, 120 aa, synthetic construct
  REGION 1..120: note = Synthetic polypeptide
QVQLVESGGGVVQPGRSLRLSCAASGFTFSHFAMHWVRQAPGKGLEWVAVISFFGGNKYYAESVKGRFTISRDNSKNTLYLQMNSLRAEDTALFYCARGYDFLTGYPDYWGQGTLVTVSS

SEQ ID NO: 280 - protein, 120 aa, synthetic construct
  REGION 1..120: note = Synthetic polypeptide
QVQLVESGGGVVQPGRSLRLSCAASGFTFSHYAMHWVRQAPGKGLEWVAVISFMGTNKYYAESVKGRFTISRDNSKNTLYLQMNSLRAEDTALFYCARGYDFLTGYPDYWGQGTLVTVSS

SEQ ID NO: 281 - protein, 120 aa, synthetic construct
  REGION 1..120: note = Synthetic polypeptide
QVQLVESGGGVVQPGRSLRLSCAASGFTFSYFAMHWVRQAPGKGLEWVAVISHRGTNKYYAESVKGRFTISRDNSKNTLYLQMNSLRAEDTALFYCARGYDLLSGYPDYWGQGTLVTVSS

SEQ ID NO: 282 - protein, 120 aa, synthetic construct
  REGION 1..120: note = Synthetic polypeptide
QVQLVESGGGVVQPGRSLRLSCAASGFTFSRFAMHWVRQAPGKGLEWVGVINYRGHGKYYAESVKGRFTVSRDNSKNTLYLQMNSLRAEDTALFYCARGYDVLTGYPDYWGQGTLVTVSS

SEQ ID NO: 283 - protein, 120 aa, synthetic construct
  REGION 1..120: note = Synthetic polypeptide
QVQLVESGGGVVQPGRSLRLSCAASGFTFSRFAMHWVRQAPGKGLEWVGVISFSGGSKYYAESVKGRFTLSRDNSKNTLYLQMNSLRAEDTALFYCARGYDVLTGYPDYWGQGTLVTVSS

SEQ ID NO: 284 - protein, 120 aa, synthetic construct
  REGION 1..120: note = Synthetic polypeptide
QVQLVESGGGVVQPGRSLRLSCAASGFTFSRFAMHWVRQAPGKGLEWVGVISYTGQFKYYAESVKGRFTVSRDNSKNTLYLQMNSLRAEDTALFYCARGYDVLTGYPDYWGQGTLVTVSS

SEQ ID NO: 285 - protein, 120 aa, synthetic construct
  REGION 1..120: note = Synthetic polypeptide
QVQLVESGGGVVQPGRSLRLSCAASGFTFSRFAMHWVRQAPGKGLEWVGVISYTGAQKYYAESVKGRFTMSRDNSKNTLYLQMNSLRAEDTALFYCARGYDVLTGYPDYWGQGTLVTVSS

SEQ ID NO: 286 - protein, 120 aa, synthetic construct
  REGION 1..120: note = Synthetic polypeptide
QVQLVESGGGVVQPGRSLRLSCAASGFTFSRFAMHWVRQAPGKGLEWVGVISYSGASKYYAESVKGRFTMSRDNSKNTLYLQMNSLRAEDTALFYCARGYDVLTGYPDYWGQGTLVTVSS

SEQ ID NO: 287 - protein, 120 aa, synthetic construct
  REGION 1..120: note = Synthetic polypeptide
QVQLVESGGGVVQPGRSLRLSCAASGFTFSRFAMHWVRQAPGKGLEWVAVISYSGAFKYYAESVKGRFTVSRDNSKNTLYLQMNSLRAEDTALFYCARGYDVLTGYPDYWGQGTLVTVSS

SEQ ID NO: 288 - protein, 120 aa, synthetic construct
  REGION 1..120: note = Synthetic polypeptide
QVQLVESGGGVVQPGRSLRLSCAASGFTFSRFAMHWVRQAPGKGLEWVGVITYTGGAKYYAESVKGRFTISRDNSKNTLYLQMNSLRAEDTALFYCARGYDVLTGYPDYWGQGTLVTVSS

SEQ ID NO: 289 - protein, 120 aa, synthetic construct
  REGION 1..120: note = Synthetic polypeptide
QVQLVESGGGVVQPGRSLRLSCAASGFTFSRFAMHWVRQAPGKGLEWVGVINFQGTTKYYAESVKGRFTISRDNSKNTLYLQMNSLRAEDTALFYCARGYDVLTGYPDYWGQGTLVTVSS

SEQ ID NO: 290 - protein, 120 aa, synthetic construct
  REGION 1..120: note = Synthetic polypeptide
QVQLVESGGGVVQPGRSLRLSCAASGFTFSRFAMHWVRQAPGKGLEWVGVISYSGDLKYYAESVKGRFTVSRDNSKNTLYLQMNSLRAEDTALFYCARGYDVLTGYPDYWGQGTLVTVSS

SEQ ID NO: 291 - protein, 120 aa, synthetic construct
  REGION 1..120: note = Synthetic polypeptide
QVQLVESGGGVVQPGRSLRLSCAASGFTFSRFAMHWVRQAPGKGLEWVGVINYFGDAKYYAESVKGRFTISRDNSKNTLYLQMNSLRAEDTALFYCARGYDVLTGYPDYWGQGTLVTVSS

SEQ ID NO: 292 - protein, 120 aa, synthetic construct
  REGION 1..120: note = Synthetic polypeptide
QVQLVESGGGVVQPGRSLRLSCAASGFTFSFYAMHWVRQAPGKGLEWVAVISSFGSNKYYAESVKGRFTISRDNSKNTLYLQMNSLRAEDTALFYCARGYDLLTGYPDYWGQGTLVTVSS

SEQ ID NO: 293 - protein, 120 aa, synthetic construct
  REGION 1..120: note = Synthetic polypeptide
QVQLVESGGGVVQPGRSLRLSCAASGFTFSYYAMHWVRQAPGKGLEWVAVISYSGSNKYYAESVKGRFTISRDNSKNTLYLQMNSLRAEDTALFYCARGYDLLTGYPDYWGQGTLVTVSS

SEQ ID NO: 294 - protein, 120 aa, synthetic construct
  REGION 1..120: note = Synthetic polypeptide
QVQLVESGGGVVQPGRSLRLSCAASGFTFSYYAMHWVRQAPGKGLEWVAVISHYGTNKYYAESVKGRFTISRDNSKNTLYLQMNSLRAEDTALFYCARGYDPLTGYPDYWGQGTLVTVSS

SEQ ID NO: 295 - protein, 120 aa, synthetic construct
  REGION 1..120: note = Synthetic polypeptide
QVQLVESGGGVVQPGRSLRLSCAASGFTFSHYAMHWVRQAPGKGLEWVAVISYQGSNKYYAESVKGRFTISRDNSKNTLYLQMNSLRAEDTALFYCARGYDLLTGYPDYWGQGTLVTVSS

SEQ ID NO: 296 - protein, 123 aa, synthetic construct
  REGION 1..123: note = Synthetic polypeptide
QVQLQQSGPGLVKPSQTLSLTCAISGDSVSSNSATWNWIRQSPSRGLEWLGRTYYRSKWSNHYAVSVKSRITINPDTSKSQFSLQLNSVTPEDTAVYYCARGTWKQLWFLDHWGQGTLVTVSS

SEQ ID NO: 297 - protein, 123 aa, synthetic construct
  REGION 1..123: note = Synthetic polypeptide
QVQLQQSGPGLVKPSQTLSLTCAISGDSVSNRLATWNWIRQSPSRGLEWLGRTYYRGKWKNHYAVSVKSRITINPDTSKSQFSLQLNSVTPEDTAVYYCARGTWNQDWFLDHWGQGTLVTVSS

SEQ ID NO: 298 - protein, 123 aa, synthetic construct
  REGION 1..123: note = Synthetic polypeptide
QVQLQQSGPGLVKPSQTLSLTCAISGDSVSNRLATWNWIRQSPSRGLEWLGRTYYRGKWKNHYAVSVKSRITINPDTSKSQFSLQLNSVTPEDTAVYYCARGTWDQDWFLDHWGQGTLVTVSS

SEQ ID NO: 299 - protein, 123 aa, synthetic construct
  REGION 1..123: note = Synthetic polypeptide
QVQLQQSGPGLVKPSQTLSLTCAISGDSVSSRQATWNWIRQSPSRGLEWLGRTYYRGKWKNHYAVSVKSRITINPDTSKSQFSLQLNSVTPEDTAVYYCARGTWEQDWFLDHWGQGTLVTVSS

SEQ ID NO: 300 - protein, 123 aa, synthetic construct
  REGION 1..123: note = Synthetic polypeptide
QVQLQQSGPGLVKPSQTLSLTCAISGDSVSNKQATWNWIRQSPSRGLEWLGRTYYRGKWKNHYAVSVKSRITINPDTSKSQFSLQLNSVTPEDTAVYYCARGMWNQNWFLDHWGQGTLVTVSS

SEQ ID NO: 301 - protein, 123 aa, synthetic construct
  REGION 1..123: note = Synthetic polypeptide
QVQLQQSGPGLVKPSQTLSLTCAISGDSVSNKQATWNWIRQSPSRGLEWLGRTYYRGQWKNHYAVSVKSRITINPDTSKSQFSLQLNSVTPEDTAVYYCARGTWIGDWFMDHWGQGTLVTVSS

SEQ ID NO: 302 - protein, 123 aa, synthetic construct
  REGION 1..123: note = Synthetic polypeptide
QVQLQQSGPGLVKPSQTLSLTCAISGDSVSNKQATWNWIRQSPSRGLEWLGRTYYRGKWKNHYAVSVKSRITINPDTSKSQFSLQLNSVTPEDTAVYYCARGTWIQDWFLDHWGQGTLVTVSS

SEQ ID NO: 303 - protein, 123 aa, synthetic construct
  REGION 1..123: note = Synthetic polypeptide
QVQLQQSGPGLVKPSQTLSLTCAISGDSVSSNHATWNWIRQSPSRGLEWLGRTYYRGKWKNHYAVSVKSRITINPDTSKSQFSLQLNSVTPEDTAVYYCARGTWDQDWFLDHWGQGTLVTVSS

SEQ ID NO: 304 - protein, 123 aa, synthetic construct
  REGION 1..123: note = Synthetic polypeptide
QVQLQQSGPGLVKPSQTLSLTCAISGDSVSSRYATWNWIRQSPSRGLEWLGRTYYRGQWKNHYAVSVKSRITINPDTSKSQFSLQLNSVTPEDTAVYYCARGMWNQNWFLDHWGQGTLVTVSS

SEQ ID NO: 305 - protein, 123 aa, synthetic construct
  REGION 1..123: note = Synthetic polypeptide
QVQLQQSGPGLVKPSQTLSLTCAISGDSVSNKQATWNWIRQSPSRGLEWLGRTYYRGKWKNHYAVSVKSRITINPDTSKSQFSLQLNSVTPEDTAVYYCARGMWSGDWFLDHWGQGTLVTVSS

SEQ ID NO: 306 - protein, 123 aa, synthetic construct
  REGION 1..123: note = Synthetic polypeptide
QVQLQQSGPGLVKPSQTLSLTCAISGDSVSSHVATWNWIRQSPSRGLEWLGRTYYRGKWKNHYAVSVKSRITINPDTSKSQFSLQLNSVTPEDTAVYYCARGMWSEDWFLDHWGQGTLVTVSS

SEQ ID NO: 307 - protein, 123 aa, synthetic construct
  REGION 1..123: note = Synthetic polypeptide
QVQLQQSGPGLVKPSQTLSLTCAISGDSVSSRQATWNWIRQSPSRGLEWLGRTYYRGKWKNHYAVSVKSRITINPDTSKSQFSLQLNSVTPEDTAVYYCARGMWQGNWFLDHWGQGTLVTVSS

SEQ ID NO: 308 - protein, 123 aa, synthetic construct
  REGION 1..123: note = Synthetic polypeptide
QVQLQQSGPGLVKPSQTLSLTCAISGDSVSNHQATWNWIRQSPSRGLEWLGRTYYRGKWKNHYAVSVKSRITINPDTSKSQFSLQLNSVTPEDTAVYYCARGTWIQDWFLDHWGQGTLVTVSS

SEQ ID NO: 309 - protein, 123 aa, synthetic construct
  REGION 1..123: note = Synthetic polypeptide
QVQLQQSGPGLVKPSQTLSLTCAISGDSVSSRDATWNWIRQSPSRGLEWLGRTYYRGKWKNHYAVSVKSRITINPDTSKSQFSLQLNSVTPEDTAVYYCARGQWNEDWFLDHWGQGTLVTVSS

SEQ ID NO: 310 - protein, 123 aa, synthetic construct
  REGION 1..123: note = Synthetic polypeptide
QVQLQQSGPGLVKPSQTLSLTCAISGDSVSNRLATWNWIRQSPSRGLEWLGRTYYRGKWKNHYAVSVKSRITINPDTSKSQFSLQLNSVTPEDTAVYYCARGRWEGDWFFDHWGQGTLVTVSS

SEQ ID NO: 311 - protein, 123 aa, synthetic construct
  REGION 1..123: note = Synthetic polypeptide
QVQLQQSGPGLVKPSQTLSLTCAISGDSVSNKQATWNWIRQSPSRGLEWLGRTYFRRTWKNHYAVSVKSRITINPDTSKSQFSLQLNSVTPEDTAVYYCARGMWSEDWFLDHWGQGTLVTVSS

SEQ ID NO: 312 - protein, 123 aa, synthetic construct
  REGION 1..123: note = Synthetic polypeptide
QVQLQQSGPGLVKPSQTLSLTCAISGDSVSNRLATWNWIRQSPSRGLEWLGRTYYRGKWKNHYAVSVKSRITINPDTSKSQFSLQLNSVTPEDTAVYYCARGVWIGNWFLDHWGQGTLVTVSS

SEQ ID NO: 313 - protein, 106 aa, synthetic construct
  REGION 1..106: note = Synthetic polypeptide
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 314 - protein, 106 aa, synthetic construct
  REGION 1..106: note = Synthetic polypeptide
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 315 - protein, 105 aa, synthetic construct
  REGION 1..105: note = Synthetic polypeptide
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 316 - protein, 106 aa, synthetic construct
  REGION 1..106: note = Synthetic polypeptide
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 317 - protein, 106 aa, synthetic construct
  REGION 1..106: note = Synthetic polypeptide
GQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 318 - protein, 106 aa, synthetic construct
  REGION 1..106: note = Synthetic polypeptide
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 319 - protein, 107 aa, synthetic construct [sequence omitted]

SEQ ID NO: 320 - protein, 330 aa, synthetic construct
  REGION 1..330: note = Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 321 - protein, 330 aa, synthetic construct [sequence omitted]

SEQ ID NO: 322 - protein, 330 aa, synthetic construct
  REGION 1..330: note = Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 323 - protein, 330 aa, synthetic construct
  REGION 1..330: note = Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 324 - protein, 330 aa, synthetic construct
  REGION 1..330: note = Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 325 - protein, 330 aa, synthetic construct
  REGION 1..330: note = Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 326 - protein, 326 aa, synthetic construct
  REGION 1..326: note = Synthetic polypeptide
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 327 - protein, 330 aa, synthetic construct; identical to SEQ ID NO: 325
  REGION 1..330: note = Synthetic polypeptide

SEQ ID NO: 328 - DNA, 326 bp, synthetic construct
  misc_feature 1..326: note = Synthetic polynucleotide
  source 1..326: mol_type = other DNA
gagatcgtacttactcagtcacccgccacattgtccctgagcccgggtgaacgggcgaccctcagctgccgagcatcccagtccgtcggacgatcattgcactggtaccaacaaaaaccgggccaggcccccagacttctgatcaagtatgcgtcacagagcttgtcgggtattcccgctcgcttttcggggtcgggatccgggacagatttcacgctcacaatctcctcgctggaacccgaggacttcgcggtctactattgtcatcagtcatcgaggttgcctttcacgtttggaccagggaccaaggtggacattaagcgtac

SEQ ID NO: 329 - DNA, 326 bp, synthetic construct
  misc_feature 1..326: note = Synthetic polynucleotide
  source 1..326: mol_type = other DNA
gatatccagctcactcaatcgccatcatttctctccgcttcggtaggcgaccgggtcacgatcacatgcagggcgtcgcaaagcattgggaggtcgttgcattggtatcagcagaaacccggaaaggccccgaaacttctgatcaaatacgcatcacaaagcttgagcggtgtgccgtcgcgcttctccggttccggaagcggaacggaattcacgcttacaatctcctcactgcagcccgaggatttcgcgacctattactgtcaccagtcatccagactcccgtttacttttggccctgggaccaaggtggacattaagcgtac

SEQ ID NO: 330 - DNA, 321 bp, synthetic construct
  misc_feature 1..321: note = Synthetic polynucleotide
  source 1..321: mol_type = other DNA
gagatcgtacttactcagtcacccgccacattgtccctgagcccgggtgaacgggcgaccctcagctgccgagcatccaagtccgtcggacgatcattgcactggtaccaacaaaaaccgggccaggcccccagacttctgatcaagtatgcgtcacagagcttgtcgggtattcccgctcgcttttcggggtcgggatccgggacagatttcacgctcacaatctcctcgctggaacccgaggacttcgcggtctactattgtcatcagtcatcgaggttgcctttcacgtttggaccagggaccaaggtggacattaag

SEQ ID NO: 331 - DNA, 321 bp, synthetic construct
  misc_feature 1..321: note = Synthetic polynucleotide
  source 1..321: mol_type = other DNA
gagatcgtacttactcagtcacccgccacattgtccctgagcccgggtgaacgggcgaccctcagctgccgagcatcccagtccgtcggacgatcattgcactggtaccaacaaaaaccgggccaggcccccagacttctgatcaagtatgcgtcacagagcttgtcgggtattcccgctcgcttttcggggtcgggatccgggacagatttcacgctcacaatctcctcgctggaacccgaggacttcgcggtctactattgtcatcagtcatcgaggttgcctttcacgtttggaccagggaccaaggtggacattaag

SEQ ID NO: 332 - DNA, 321 bp, synthetic construct
  misc_feature 1..321: note = Synthetic polynucleotide
  source 1..321: mol_type = other DNA
gagatcgtacttactcagtcacccgccacattgtccctgagcccgggtgaacgggcgaccctcagctgccgagcatcccagtccgtctggcgatcattgcactggtaccaacaaaaaccgggccaggcccccagacttctgatcaagtatgcgtcacagagcttgtcgggtattcccgctcgcttttcggggtcgggatccgggacagatttcacgctcacaatctcctcgctggaacccgaggacttcgcggtctactattgtcatcagtcatcgaggttgcctttcacgtttggaccagggaccaaggtggacattaag

SEQ ID NO: 333 - DNA, 321 bp, synthetic construct
  misc_feature 1..321: note = Synthetic polynucleotide
  source 1..321: mol_type = other DNA
gagatcgtacttactcagtcacccgccacattgtccctgagcccgggtgaacgggcgaccctcagctgccgagcatcccagtccgtcggacgaaacttgcactggtaccaacaaaaaccgggccaggcccccagacttctgatcaagtatgcgtcacagagcttgtcgggtattcccgctcgcttttcggggtcgggatccgggacagatttcacgctcacaatctcctcgctggaacccgaggacttcgcggtctactattgtcatcagtcatcgaggttgcctttcacgtttggaccagggaccaaggtggacattaag

SEQ ID NO: 334 - DNA, 321 bp, synthetic construct
  misc_feature 1..321: note = Synthetic polynucleotide
  source 1..321: mol_type = other DNA
gagatcgtacttactcagtcacccgccacattgtccctgagcccgggtgaacgggcgaccctcagctgccgagcatcccagtccgtcggacgatcattgcactggtaccaacaaaaaccgggccaggcccccagacttctgatcaagtatgcgtcacagagcttgtcgggtattcccgctcgcttttcggggtcgggatccgggacagatttcacgctcacaatctcctcgctggaacccgaggacttcgcggtctactattgtcatcagtcatcgatgttgcctttcacgtttggaccagggaccaaggtggacattaag

SEQ ID NO: 335 - DNA, 321 bp, synthetic construct
  misc_feature 1..321: note = Synthetic polynucleotide
  source 1..321: mol_type = other DNA
gagatcgtacttactcagtcacccgccacattgtccctgagcccgggtgaacgggcgaccctcagctgccgagcatccaagtccgtctggcgatcattgcactggtaccaacaaaaaccgggccaggcccccagacttctgatcaagtatgcgtcacagagcttgtcgggtattcccgctcgcttttcggggtcgggatccgggacagatttcacgctcacaatctcctcgctggaacccgaggacttcgcggtctactattgtcatcagtcatcgaggttgcctttcacgtttggaccagggaccaaggtggacattaag

SEQ ID NO: 336 - DNA, 321 bp, synthetic construct
  misc_feature 1..321: note = Synthetic polynucleotide
  source 1..321: mol_type = other DNA
gagatcgtacttactcagtcacccgccacattgtccctgagcccgggtgaacgggcgaccctcagctgccgagcatccaagtccgtcggacgaaacttgcactggtaccaacaaaaaccgggccaggcccccagacttctgatcaagtatgcgtcacagagcttgtcgggtattcccgctcgcttttcggggtcgggatccgggacagatttcacgctcacaatctcctcgctggaacccgaggacttcgcggtctactattgtcatcagtcatcgaggttgcctttcacgtttggaccagggaccaaggtggacattaag

SEQ ID NO: 337 - DNA, 321 bp, synthetic construct
  misc_feature 1..321: note = Synthetic polynucleotide
  source 1..321: mol_type = other DNA
gagatcgtacttactcagtcacccgccacattgtccctgagcccgggtgaacgggcgaccctcagctgccgagcatccaagtccgtcggacgatcattgcactggtaccaacaaaaaccgggccaggcccccagacttctgatcaagtatgcgtcacagagcttgtcgggtattcccgctcgcttttcggggtcgggatccgggacagatttcacgctcacaatctcctcgctggaacccgaggacttcgcggtctactattgtcatcagtcatcgatgttgcctttcacgtttggaccagggaccaaggtggacattaag

SEQ ID NO: 338 - DNA, 325 bp, synthetic construct
  misc_feature 1..325: note = Synthetic polynucleotide
  source 1..325: mol_type = other DNA
gagatcgtacttactcagtcacccgccacattgtccctgagcccgggtgaacgggcgaccctcagctgccgagcatccaaatccgtcgggtggagcttgcactggtaccaacaaaaaccgggccaggcccccagacttctgatcaagtatgcgtcacagagcttgtcgggtattcccgctcgcttttcggggtcgggatccgggacagatttcacgctcacaatctcctcgctggaacccgaggacttcgcggtctactattgtcatcagtcatcgaggttgcctttcacgtttggaccagggaccaaggtggacattaagcgta

SEQ ID NO: 339 - DNA, 325 bp, synthetic construct
  misc_feature 1..325: note = Synthetic polynucleotide
  source 1..325: mol_type = other DNA
gagatcgtacttactcagtcacccgccacattgtccctgagcccgggtgaacgggcgaccctcagctgccgagcatccaaatccgtcgggtacagcttgcactggtaccaacaaaaaccgggccaggcccccagacttctgatcaagtatgcgtcacagagcttgtcgggtattcccgctcgcttttcggggtcgggatccgggacagatttcacgctcacaatctcctcgctggaacccgaggacttcgcggtctactattgtcatcagtcatcgaggttgcctttcacgtttggaccagggaccaaggtggacattaagcgta

SEQ ID NO: 340 - DNA, 325 bp, synthetic construct
  misc_feature 1..325: note = Synthetic polynucleotide
  source 1..325: mol_type = other DNA
gagatcgtacttactcagtcacccgccacattgtccctgagcccgggtgaacgggcgaccctcagctgccgagcatccaaagccgtcgggtggagcttgcactggtaccaacaaaaaccgggccaggcccccagacttctgatcaagtatgcgtcacagagcttgtcgggtattcccgctcgcttttcggggtcgggatccgggacagatttcacgctcacaatctcctcgctggaacccgaggacttcgcggtctactattgtcatcagtcatcgaggttgcctttcacgtttggaccagggaccaaggtggacattaagcgta

SEQ ID NO: 341 - DNA, 325 bp, synthetic construct
  misc_feature 1..325: note = Synthetic polynucleotide
  source 1..325: mol_type = other DNA
gagatcgtacttactcagtcacccgccacattgtccctgagcccgggtgaacgggcgaccctcagctgccgagcatccaaatcagtcggtcagtctttgcactggtaccaacaaaaaccgggccaggcccccagacttctgatcaagtatgcgtcacagagcttgtcgggtattcccgctcgcttttcggggtcgggatccgggacagatttcacgctcacaatctcctcgctggaacccgaggacttcgcggtctactattgtcatcagtcatcgcgtttgcctttcacgtttggaccagggaccaaggtggacattaagcgta

SEQ ID NO: 342 - DNA, 325 bp, synthetic construct
  misc_feature 1..325: note = Synthetic polynucleotide
  source 1..325: mol_type = other DNA
gagatcgtacttactcagtcacccgccacattgtccctgagcccgggtgaacgggcgaccctcagctgccgagcatcccgttcagtcggtctggctttgcactggtaccaacaaaaaccgggccaggcccccagacttctgatcaagtatgcgtcacagagcttgtcgggtattcccgctcgcttttcggggtcgggatccgggacagatttcacgctcacaatctcctcgctggaacccgaggacttcgcggtctactattgtcatcagtcatcgttcttgcctttcacgtttggaccagggaccaaggtggacattaagcgta

SEQ ID NO: 343 - DNA, 326 bp, synthetic construct
  misc_feature 1..326: note = Synthetic polynucleotide
  source 1..326: mol_type = other DNA
gacatccagatgacccagtctccatcctccctgtctgcatctgtaggagacagaatcaccatcacttgccgggcaagtcagagcattagcaggtatttaaattggtatcaacagaaaccagggaaagcccctaaactcctgatctatgctgcatccagtttgcaaagtgggatcccatcaaggttcagcggcagtggatctgggacagatttcactctcaccatcaacagtctgcaacctgaagattttgcaacttacttctgtcaacagagttacagtcccccattcactttcggccctgggaccaaagtggatatcaaacgtac

SEQ ID NO: 344 - DNA, 321 bp, synthetic construct
  misc_feature 1..321: note = Synthetic polynucleotide
  source 1..321: mol_type = other DNA
gacatccagatgacccagtctccatcctccctgtctgcatctgtaggagacagaatcaccatcacttgccgggcaagtcagagcattagcaggtatttaaattggtatcaacagaaaccagggaaagcccctaaactcctgatcttcgctggtcagcgtttgcaaagtgggatcccatcaaggttcagcggcagtggatctgggacagatttcactctcaccatcaacagtctgcaacctgaagattttgcaacttacttctgtcaacaggctatcggtatgccatacactttcggccctgggaccaaagtggatatcaaa

SEQ ID NO: 345 - DNA, 321 bp, synthetic construct
  misc_feature 1..321: note = Synthetic polynucleotide
  source 1..321: mol_type = other DNA
gacatccagatgacccagtctccatcctccctgtctgcatctgtaggagacagaatcaccatcacttgccgggcaagtcagtacattgttcgttacttaaactggtatcaacagaaaccagggaaagcccctaaactcctgatctacgctgctcatcatttgcaaagtgggatcccatcaaggttcagcggcagtggatctgggacagatttcactctcaccatcaacagtctgcaacctgaagattttgcaacttacttctgtcaacaggctatccaggaaccatacactttcggccctgggaccaaagtggatatcaaa

SEQ ID NO: 346 - DNA, 321 bp, synthetic construct
  misc_feature 1..321: note = Synthetic polynucleotide
  source 1..321: mol_type = other DNA
gacatccagatgacccagtctccatcctccctgtctgcatctgtaggagacagaatcaccatcacttgccgggcaagtcagactattgttcgttacttaaactggtatcaacagaaaccagggaaagcccctaaactcctgatcttcgctggtcagcgtttgcaaagtgggatcccatcaaggttcagcggcagtggatctgggacagatttcactctcaccatcaacagtctgcaacctgaagattttgcaacttacttctgtcaacaggctatcatcaacccatacactttcggccctgggaccaaagtggatatcaaa

SEQ ID NO: 347 - DNA, 321 bp, synthetic construct
  misc_feature 1..321: note = Synthetic polynucleotide
  source 1..321: mol_type = other DNA
gacatccagatgacccagtctccatcctccctgtctgcatctgtaggagacagaatcaccatcacttgccgggcaagtcagtacattgttcgttacttaaactggtatcaacagaaaccagggaaagcccctaaactcctgatctacgctgctaacatgttgcaaagtgggatcccatcaaggttcagcggcagtggatctgggacagatttcactctcaccatcaacagtctgcaacctgaagattttgcaacttacttctgtcaacaggctatcaaccagccatacactttcggccctgggaccaaagtggatatcaaa

SEQ ID NO: 348 - DNA, 321 bp, synthetic construct
  misc_feature 1..321: note = Synthetic polynucleotide
  source 1..321: mol_type = other DNA
gacatccagatgacccagtctccatcctccctgtctgcatctgtaggagacagaatcaccatcacttgccgggcaagtcagaaaattgctcgttacttagtttggtatcaacagaaaccagggaaagcccctaaactcctgatctacgctgctaacatgttgcaaagtgggatcccatcaaggttcagcggcagtggatctgggacagatttcactctcaccatcaacagtctgcaacctgaagattttgcaacttacttctgtcaacagtctatccagcagccatacactttcggccctgggaccaaagtggatatcaaa

SEQ ID NO: 349 - DNA, 324 bp, synthetic construct
  misc_feature 1..324: note = Synthetic polynucleotide
  source 1..324: mol_type = other DNA
gacatccagatgacccagtctccatcctccctgtctgcatctgtaggagacagaatcaccatcacttgccgggcaagtcagagcattagcaggtatttaaattggtatcaacagaaaccagggaaagcccctaaactcctgatcttcgctggtcagcgtttgcaaagtgggatcccatcaaggttcagcggcagtggatctgggacagatttcactctcaccatcaacagtctgcaacctgaagattttgcaacttacttctgtcaacaggctatccagcagccatacactttcggccctgggaccaaagtggatatcaaacgt

SEQ ID NO: 350 - DNA, 321 bp, synthetic construct
  misc_feature 1..321: note = Synthetic polynucleotide
  source 1..321: mol_type = other DNA
gacatccagatgacccagtctccatcctccctgtctgcatctgtaggagacagaatcaccatcacttgccgggcaagtcagtacattgttcgttacttaaactggtatcaacagaaaccagggaaagcccctaaactcctgatctacgcttcttacaacttgcaaagtgggatcccatcaaggttcagcggcagtggatctgggacagatttcactctcaccatcaacagtctgcaacctgaagattttgcaacttacttctgtcaacaggctatcatggctccatacactttcggccctgggaccaaagtggatatcaaa

SEQ ID NO: 351 - DNA, 321 bp, synthetic construct
  misc_feature 1..321: note = Synthetic polynucleotide
  source 1..321: mol_type = other DNA
gacatccagatgacccagtctccatcctccctgtctgcatctgtaggagacagaatcaccatcacttgccgggcaagtcagcctattgctcagtacttaaactggtatcaacagaaaccagggaaagcccctaaactcctgatctacgctggtcgttacttgcaaagtgggatcccatcaaggttcagcggcagtggatctgggacagatttcactctcaccatcaacagtctgcaacctgaagattttgcaacttacttctgtcaacaggctatccagaacccatacactttcggccctgggaccaaagtggatatcaaa

SEQ ID NO: 352 - DNA, 321 bp, synthetic construct
  misc_feature 1..321: note = Synthetic polynucleotide
  source 1..321: mol_type = other DNA
gacatccagatgacccagtctccatcctccctgtctgcatctgtaggagacagaatcaccatcacttgccgggcaagtcagagcattagcaggtatttaaattggtatcaacagaaaccagggaaagcccctaaactcctgatcttcgctggtcagcgtttgcaaagtgggatcccatcaaggttcagcggcagtggatctgggacagatttcactctcaccatcaacagtctgcaacctgaagattttgcaacttacttctgtcaacaggctatcgttcagccatacactttcggccctgggaccaaagtggatatcaaa

SEQ ID NO: 353 - DNA, 321 bp, synthetic construct
  misc_feature 1..321: note = Synthetic polynucleotide
  source 1..321: mol_type = other DNA
gacatccagatgacccagtctccatcctccctgtctgcatctgtaggagacagaatcaccatcacttgccgggcaagtcagccgatttctcgttacttatcttggtatcaacagaaaccagggaaagcccctaaactcctgatcttcgctggtcagcgtttgcaaagtgggatcccatcaaggttcagcggcagtggatctgggacagatttcactctcaccatcaacagtctgcaacctgaagattttgcaacttacttctgtcaacaggctatctctatcccatacactttcggccctgggaccaaagtggatatcaaa

SEQ ID NO: 354 - DNA, 321 bp, synthetic construct
  misc_feature 1..321: note = Synthetic polynucleotide
  source 1..321: mol_type = other DNA
gacatccagatgacccagtctccatcctccctgtctgcatctgtaggagacagaatcaccatcacttgccgggcaagtcagcagattgctcgttacttaaactggtatcaacagaaaccagggaaagcccctaaactcctgatctacgcttcttacaacttgcaaagtgggatcccatcaaggttcagcggcagtggatctgggacagatttcactctcaccatcaacagtctgcaacctgaagattttgcaacttacttctgtcaacaggctatcatccagccatacactttcggccctgggaccaaagtggatatcaaa

SEQ ID NO: 355 - DNA, 321 bp, synthetic construct
  misc_feature 1..321: note = Synthetic polynucleotide
  source 1..321: mol_type = other DNA
gacatccagatgacccagtctccatcctccctgtctgcatctgtaggagacagaatcaccatcacttgccgggcaagtcagagcattagcaggtatttaaattggtatcaacagaaaccagggaaagcccctaaactcctgatctacgcttcttacaacttgcaaagtgggatcccatcaaggttcagcggcagtggatctgggacagatttcactctcaccatcaacagtctgcaacctgaagattttgcaacttacttctgtcaacaggctatccagaacccatacactttcggccctgggaccaaagtggatatcaaa

SEQ ID NO: 356 - DNA, 321 bp, synthetic construct
  misc_feature 1..321: note = Synthetic polynucleotide
  source 1..321: mol_type = other DNA
gacatccagatgacccagtctccatcctccctgtctgcatctgtaggagacagaatcaccatcacttgccgggcaagtcagactattgttcgttacttaaactggtatcaacagaaaccagggaaagcccctaaactcctgatcttcgctggtcagcgtttgcaaagtgggatcccatcaaggttcagcggcagtggatctgggacagatttcactctcaccatcaacagtctgcaacctgaagattttgcaacttacttctgtcaacagtctatccagactccatacactttcggccctgggaccaaagtggatatcaaa

SEQ ID NO: 357 - DNA, 321 bp, synthetic construct
  misc_feature 1..321: note = Synthetic polynucleotide
  source 1..321: mol_type = other DNA
gacatccagatgacccagtctccatcctccctgtctgcatctgtaggagacagaatcaccatcacttgccgggcaagtcagagcattagcaggtatttaaattggtatcaacagaaaccagggaaagcccctaaactcctgatctacgctggtcgtatcttgcaaagtgggatcccatcaaggttcagcggcagtggatctgggacagatttcactctcaccatcaacagtctgcaacctgaagattttgcaacttacttctgtcaacaggctatcatcaacccatacactttcggccctgggaccaaagtggatatcaaa

SEQ ID NO: 358 - DNA, 321 bp, synthetic construct
  misc_feature 1..321: note = Synthetic polynucleotide
  source 1..321: mol_type = other DNA
gacatccagatgacccagtctccatcctccctgtctgcatctgtaggagacagaatcaccatcacttgccgggcaagtcagcgtattgctcgttacttaaactggtatcaacagaaaccagggaaagcccctaaactcctgatcttcgctggttctatcttgcaaagtgggatcccatcaaggttcagcggcagtggatctgggacagatttcactctcaccatcaacagtctgcaacctgaagattttgcaacttacttctgtcaacaggctatccagaacccatacactttcggccctgggaccaaagtggatatcaaa

SEQ ID NO: 359 - DNA, 321 bp, synthetic construct
  misc_feature 1..321: note = Synthetic polynucleotide
  source 1..321: mol_type = other DNA
gacatccagatgacccagtctccatcctccctgtctgcatctgtaggagacagaatcaccatcacttgccgggcaagtcagagcattagcaggtatttaaattggtatcaacagaaaccagggaaagcccctaaactcctgatctacgcttcttacaacttgcaaagtgggatcccatcaaggttcagcggcagtggatctgggacagatttcactctcaccatcaacagtctgcaacctgaagattttgcaacttacttctgtcaacagtctatccagcagccatacactttcggccctgggaccaaagtggatatcaaa

SEQ ID NO: 360 - DNA, 321 bp, synthetic construct
  misc_feature 1..321: note = Synthetic polynucleotide
  source 1..321: mol_type = other DNA
gacatccagatgacccagtctccatcctccctgtctgcatctgtaggagacagaatcaccatcacttgccgggcaagtcagtacattgttcgttacttaaactggtatcaacagaaaccagggaaagcccctaaactcctgatctacgcttcttacaacttgcaaagtgggatcccatcaaggttcagcggcagtggatctgggacagatttcactctcaccatcaacagtctgcaacctgaagattttgcaacttacttctgtcaacaggctatccagcagccatacactttcggccctgggaccaaagtggatatcaaa

SEQ ID NO: 361 - DNA, 321 bp, synthetic construct
  misc_feature 1..321: note = Synthetic polynucleotide
  source 1..321: mol_type = other DNA
gacatccagatgacccagtctccatcctccctgtctgcatctgtaggagacagaatcaccatcacttgccgggcaagtcagatgattgctcgttacttaaactggtatcaacagaaaccagggaaagcccctaaactcctgatctacgcttcttacaacttgcaaagtgggatcccatcaaggttcagcggcagtggatctgggacagatttcactctcaccatcaacagtctgcaacctgaagattttgcaacttacttctgtcaacaggctatcatcaacccatacactttcggccctgggaccaaagtggatatcaaa

SEQ ID NO: 362 - DNA, 321 bp, synthetic construct
  misc_feature 1..321: note = Synthetic polynucleotide
  source 1..321: mol_type = other DNA
gacatccagatgacccagtctccatcctccctgtctgcatctgtaggagacagaatcaccatcacttgccgggcaagtcagtacattgttcgttacttaaactggtatcaacagaaaccagggaaagcccctaaactcctgatctacggtgctcgtaacttgcaaagtgggatcccatcaaggttcagcggcagtggatctgggacagatttcactctcaccatcaacagtctgcaacctgaagattttgcaacttacttctgtcaacagtctatccagactccatacactttcggccctgggaccaaagtggatatcaaa

SEQ ID NO: 363 - DNA, 321 bp, synthetic construct
  misc_feature 1..321: note = Synthetic polynucleotide
  source 1..321: mol_type = other DNA
gacatccagatgacccagtctccatcctccctgtctgcatctgtaggagacagaatcaccatcacttgccgggcaagtcagagcattagcaggtatttaaattggtatcaacagaaaccagggaaagcccctaaactcctgatctatgctgcatccagtttgcaaagtgggatcccatcaaggttcagcggcagtggatctgggacagatttcactctcaccatcaacagtctgcaacctgaagattttgcaacttacttctgtcaacagagttacagtcccccattcactttcggccctgggaccaaagtggatatcaaa

SEQ ID NO: 364 - DNA, 362 bp, synthetic construct
  misc_feature 1..362: note = Synthetic polynucleotide
  source 1..362: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatatgatggaggaaataaatactatgcagagtccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctcctcagc

SEQ ID NO: 365 - DNA, 360 bp, synthetic construct
  misc_feature 1..360: note = Synthetic polynucleotide
  source 1..360: mol_type = other DNA
caagttcagttggtggagtctggagccgaagtagtaaagccaggagcttcagtgaaagtctcttgtaaagcaagtggattcacgtttagccgctttgccatgcattgggtgcggcaagctcccggtcaggggttggagtggatgggagttattagctatgacgggggcaataagtactacgccgagtctgttaagggtcgggtcacaatgacacgggacacctcaaccagtacactctatatggaactgtctagcctgagatccgaggacaccgctgtgtattattgcgctagggggtacgatgtattgacgggttatcctgattactgggggcaggggacactcgtaaccgtctctagt

SEQ ID NO: 366 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatataacggaggaaataaatactatgcagagtccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagt

SEQ ID NO: 367 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatatatcggaggaaataaatactatgcagagtccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagt

SEQ ID NO: 368 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatatcagggaggaaataaatactatgcagagtccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagt

SEQ ID NO: 369 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatattacggaggaaataaatactatgcagagtccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagt

SEQ ID NO: 370 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatatgatggacgcaataaatactatgcagagtccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagt

SEQ ID NO: 371 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatatgatggaaacaataaatactatgcagagtccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagt

SEQ ID NO: 372 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatatgatggaggaaataaatactatgcacgctccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagt

SEQ ID NO: 373 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatatgatggaggaaataaatactatgcagagtccgtgaagggccggttcaccatctccagagacaattccaagaacaccctgtatctgcaaatgaacagcctgagagctgaggacacggctctgttttactgtgcgagaggatacgatatcttgactggttaccccgactactggggccagggaaccctggtcaccgtctctagt

SEQ ID NO: 374 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatataacggacgcaataaatactatgcagagtccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagt

SEQ ID NO: 375 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatatatcggacgcaataaatactatgcagagtccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagt

SEQ ID NO: 376 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatatcagggacgcaataaatactatgcagagtccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagt

SEQ ID NO: 377 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatattacggacgcaataaatactatgcagagtccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagt

SEQ ID NO: 378 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatataacggaaacaataaatactatgcagagtccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagt

SEQ ID NO: 379 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatatatcggaaacaataaatactatgcagagtccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagt

SEQ ID NO: 380 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatatcagggaaacaataaatactatgcagagtccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagt

SEQ ID NO: 381 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatattacggaaacaataaatactatgcagagtccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagt

SEQ ID NO: 382 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatataacggaggaaataaatactatgcacgctccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagt

SEQ ID NO: 383 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatatatcggaggaaataaatactatgcacgctccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagt

SEQ ID NO: 384 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatatcagggaggaaataaatactatgcacgctccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagt

SEQ ID NO: 385 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatattacggaggaaataaatactatgcacgctccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagt

SEQ ID NO: 386 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatataacggaggaaataaatactatgcagagtccgtgaagggccggttcaccatctccagagacaattccaagaacaccctgtatctgcaaatgaacagcctgagagctgaggacacggctctgttttactgtgcgagaggatacgatatcttgactggttaccccgactactggggccagggaaccctggtcaccgtctctagt

SEQ ID NO: 387 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatatatcggaggaaataaatactatgcagagtccgtgaagggccggttcaccatctccagagacaattccaagaacaccctgtatctgcaaatgaacagcctgagagctgaggacacggctctgttttactgtgcgagaggatacgatatcttgactggttaccccgactactggggccagggaaccctggtcaccgtctctagt

SEQ ID NO: 388 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatatcagggaggaaataaatactatgcagagtccgtgaagggccggttcaccatctccagagacaattccaagaacaccctgtatctgcaaatgaacagcctgagagctgaggacacggctctgttttactgtgcgagaggatacgatatcttgactggttaccccgactactggggccagggaaccctggtcaccgtctctagt

SEQ ID NO: 389 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatattacggaggaaataaatactatgcagagtccgtgaagggccggttcaccatctccagagacaattccaagaacaccctgtatctgcaaatgaacagcctgagagctgaggacacggctctgttttactgtgcgagaggatacgatatcttgactggttaccccgactactggggccagggaaccctggtcaccgtctctagt

SEQ ID NO: 390 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatatgatggacgcaataaatactatgcacgctccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagt

SEQ ID NO: 391 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatatgatggaaacaataaatactatgcacgctccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagt

SEQ ID NO: 392 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatatgatggacgcaataaatactatgcagagtccgtgaagggccggttcaccatctccagagacaattccaagaacaccctgtatctgcaaatgaacagcctgagagctgaggacacggctctgttttactgtgcgagaggatacgatatcttgactggttaccccgactactggggccagggaaccctggtcaccgtctctagt

SEQ ID NO: 393 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatatgatggaaacaataaatactatgcagagtccgtgaagggccggttcaccatctccagagacaattccaagaacaccctgtatctgcaaatgaacagcctgagagctgaggacacggctctgttttactgtgcgagaggatacgatatcttgactggttaccccgactactggggccagggaaccctggtcaccgtctctagt

SEQ ID NO: 394 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatatgatggaggaaataaatactatgcacgctccgtgaagggccggttcaccatctccagagacaattccaagaacaccctgtatctgcaaatgaacagcctgagagctgaggacacggctctgttttactgtgcgagaggatacgatatcttgactggttaccccgactactggggccagggaaccctggtcaccgtctctagt

SEQ ID NO: 395 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatataacggacgcaataaatactatgcacgctccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagt

SEQ ID NO: 396 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatatatcggacgcaataaatactatgcacgctccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagt

SEQ ID NO: 397 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatatcagggacgcaataaatactatgcacgctccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagt

SEQ ID NO: 398 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatattacggacgcaataaatactatgcacgctccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagt

SEQ ID NO: 399 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatataacggaaacaataaatactatgcacgctccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagt

SEQ ID NO: 400 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatatatcggaaacaataaatactatgcacgctccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagt

SEQ ID NO: 401 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatatcagggaaacaataaatactatgcacgctccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagt

SEQ ID NO: 402 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatattacggaaacaataaatactatgcacgctccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagt

SEQ ID NO: 403 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatataacggacgcaataaatactatgcagagtccgtgaagggccggttcaccatctccagagacaattccaagaacaccctgtatctgcaaatgaacagcctgagagctgaggacacggctctgttttactgtgcgagaggatacgatatcttgactggttaccccgactactggggccagggaaccctggtcaccgtctctagt

SEQ ID NO: 404 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatatatcggacgcaataaatactatgcagagtccgtgaagggccggttcaccatctccagagacaattccaagaacaccctgtatctgcaaatgaacagcctgagagctgaggacacggctctgttttactgtgcgagaggatacgatatcttgactggttaccccgactactggggccagggaaccctggtcaccgtctctagt

SEQ ID NO: 405 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatatcagggacgcaataaatactatgcagagtccgtgaagggccggttcaccatctccagagacaattccaagaacaccctgtatctgcaaatgaacagcctgagagctgaggacacggctctgttttactgtgcgagaggatacgatatcttgactggttaccccgactactggggccagggaaccctggtcaccgtctctagt

SEQ ID NO: 406 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatattacggacgcaataaatactatgcagagtccgtgaagggccggttcaccatctccagagacaattccaagaacaccctgtatctgcaaatgaacagcctgagagctgaggacacggctctgttttactgtgcgagaggatacgatatcttgactggttaccccgactactggggccagggaaccctggtcaccgtctctagt

SEQ ID NO: 407 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatataacggaaacaataaatactatgcagagtccgtgaagggccggttcaccatctccagagacaattccaagaacaccctgtatctgcaaatgaacagcctgagagctgaggacacggctctgttttactgtgcgagaggatacgatatcttgactggttaccccgactactggggccagggaaccctggtcaccgtctctagt

SEQ ID NO: 408 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatatatcggaaacaataaatactatgcagagtccgtgaagggccggttcaccatctccagagacaattccaagaacaccctgtatctgcaaatgaacagcctgagagctgaggacacggctctgttttactgtgcgagaggatacgatatcttgactggttaccccgactactggggccagggaaccctggtcaccgtctctagt

SEQ ID NO: 409 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatatcagggaaacaataaatactatgcagagtccgtgaagggccggttcaccatctccagagacaattccaagaacaccctgtatctgcaaatgaacagcctgagagctgaggacacggctctgttttactgtgcgagaggatacgatatcttgactggttaccccgactactggggccagggaaccctggtcaccgtctctagt

SEQ ID NO: 410 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatattacggaaacaataaatactatgcagagtccgtgaagggccggttcaccatctccagagacaattccaagaacaccctgtatctgcaaatgaacagcctgagagctgaggacacggctctgttttactgtgcgagaggatacgatatcttgactggttaccccgactactggggccagggaaccctggtcaccgtctctagt

SEQ ID NO: 411 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatatgatggacgcaataaatactatgcacgctccgtgaagggccggttcaccatctccagagacaattccaagaacaccctgtatctgcaaatgaacagcctgagagctgaggacacggctctgttttactgtgcgagaggatacgatatcttgactggttaccccgactactggggccagggaaccctggtcaccgtctctagt

SEQ ID NO: 412 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatatgatggaaacaataaatactatgcacgctccgtgaagggccggttcaccatctccagagacaattccaagaacaccctgtatctgcaaatgaacagcctgagagctgaggacacggctctgttttactgtgcgagaggatacgatatcttgactggttaccccgactactggggccagggaaccctggtcaccgtctctagt

SEQ ID NO: 413 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatataacggacgcaataaatactatgcacgctccgtgaagggccggttcaccatctccagagacaattccaagaacaccctgtatctgcaaatgaacagcctgagagctgaggacacggctctgttttactgtgcgagaggatacgatatcttgactggttaccccgactactggggccagggaaccctggtcaccgtctctagt

SEQ ID NO: 414 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatatatcggacgcaataaatactatgcacgctccgtgaagggccggttcaccatctccagagacaattccaagaacaccctgtatctgcaaatgaacagcctgagagctgaggacacggctctgttttactgtgcgagaggatacgatatcttgactggttaccccgactactggggccagggaaccctggtcaccgtctctagt

SEQ ID NO: 415 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatatcagggacgcaataaatactatgcacgctccgtgaagggccggttcaccatctccagagacaattccaagaacaccctgtatctgcaaatgaacagcctgagagctgaggacacggctctgttttactgtgcgagaggatacgatatcttgactggttaccccgactactggggccagggaaccctggtcaccgtctctagt

SEQ ID NO: 416 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatattacggacgcaataaatactatgcacgctccgtgaagggccggttcaccatctccagagacaattccaagaacaccctgtatctgcaaatgaacagcctgagagctgaggacacggctctgttttactgtgcgagaggatacgatatcttgactggttaccccgactactggggccagggaaccctggtcaccgtctctagt

SEQ ID NO: 417 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatataacggaaacaataaatactatgcacgctccgtgaagggccggttcaccatctccagagacaattccaagaacaccctgtatctgcaaatgaacagcctgagagctgaggacacggctctgttttactgtgcgagaggatacgatatcttgactggttaccccgactactggggccagggaaccctggtcaccgtctctagt

SEQ ID NO: 418 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatatatcggaaacaataaatactatgcacgctccgtgaagggccggttcaccatctccagagacaattccaagaacaccctgtatctgcaaatgaacagcctgagagctgaggacacggctctgttttactgtgcgagaggatacgatatcttgactggttaccccgactactggggccagggaaccctggtcaccgtctctagt

SEQ ID NO: 419 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatatcagggaaacaataaatactatgcacgctccgtgaagggccggttcaccatctccagagacaattccaagaacaccctgtatctgcaaatgaacagcctgagagctgaggacacggctctgttttactgtgcgagaggatacgatatcttgactggttaccccgactactggggccagggaaccctggtcaccgtctctagt

SEQ ID NO: 420 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatattacggaaacaataaatactatgcacgctccgtgaagggccggttcaccatctccagagacaattccaagaacaccctgtatctgcaaatgaacagcctgagagctgaggacacggctctgttttactgtgcgagaggatacgatatcttgactggttaccccgactactggggccagggaaccctggtcaccgtctctagt

SEQ ID NO: 421 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtcactttgccatgcactgggtccgccaggctccaggcaaggggctggagtgggtggcagttatatcatatgatggaggaaataaatactatgcagagtccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagt

SEQ ID NO: 422 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatacgccatgcactgggtccgccaggctccaggcaaggggctggagtgggtggcagttatatcatatgatggaggaaataaatactatgcagagtccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagt

SEQ ID NO: 423 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcattcgatggaggaaataaatactatgcagagtccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagt

SEQ ID NO: 424 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatatgatggagccaataaatactatgcagagtccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagt

SEQ ID NO: 425 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatatgatggaggaaataaatactatgcagagtccgtgaagggccggttcaccatctccagagacaattccaagaacaccctgtatctgcaaatgaacagcctgagagctgaggacacggctctgttttactgtgcgagaggatacgatttcttgactggttaccccgactactggggccagggaaccctggtcaccgtctctagt

SEQ ID NO: 426 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtcactttgccatgcactgggtccgccaggctccaggcaaggggctggagtgggtggcagttatatcatataacggaggaaataaatactatgcagagtccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagt

SEQ ID NO: 427 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatacgccatgcactgggtccgccaggctccaggcaaggggctggagtgggtggcagttatatcatataacggaggaaataaatactatgcagagtccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagt

SEQ ID NO: 428 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcattcaacggaggaaataaatactatgcagagtccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagt

SEQ ID NO: 429 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatataacggagccaataaatactatgcagagtccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagt

SEQ ID NO: 430 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatataacggaggaaataaatactatgcagagtccgtgaagggccggttcaccatctccagagacaattccaagaacaccctgtatctgcaaatgaacagcctgagagctgaggacacggctctgttttactgtgcgagaggatacgatttcttgactggttaccccgactactggggccagggaaccctggtcaccgtctctagt

SEQ ID NO: 431 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtcactttgccatgcactgggtccgccaggctccaggcaaggggctggagtgggtggcagttatatcatatgatggacgcaataaatactatgcagagtccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagt

SEQ ID NO: 432 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatacgccatgcactgggtccgccaggctccaggcaaggggctggagtgggtggcagttatatcatatgatggacgcaataaatactatgcagagtccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagt

SEQ ID NO: 433 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcattcgatggacgcaataaatactatgcagagtccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagt

SEQ ID NO: 434 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatatgatggacgcaataaatactatgcagagtccgtgaagggccggttcaccatctccagagacaattccaagaacaccctgtatctgcaaatgaacagcctgagagctgaggacacggctctgttttactgtgcgagaggatacgatttcttgactggttaccccgactactggggccagggaaccctggtcaccgtctctagt

SEQ ID NO: 435 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtcactttgccatgcactgggtccgccaggctccaggcaaggggctggagtgggtggcagttatatcatatgatggaggaaataaatactatgcacgctccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagt

SEQ ID NO: 436 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatacgccatgcactgggtccgccaggctccaggcaaggggctggagtgggtggcagttatatcatatgatggaggaaataaatactatgcacgctccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagt

SEQ ID NO: 437 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcattcgatggaggaaataaatactatgcacgctccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagt

SEQ ID NO: 438 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatatgatggagccaataaatactatgcacgctccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagt

SEQ ID NO: 439 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatatgatggaggaaataaatactatgcacgctccgtgaagggccggttcaccatctccagagacaattccaagaacaccctgtatctgcaaatgaacagcctgagagctgaggacacggctctgttttactgtgcgagaggatacgatttcttgactggttaccccgactactggggccagggaaccctggtcaccgtctctagt

SEQ ID NO: 440 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtcactttgccatgcactgggtccgccaggctccaggcaaggggctggagtgggtggcagttatatcatatgatggaggaaataaatactatgcagagtccgtgaagggccggttcaccatctccagagacaattccaagaacaccctgtatctgcaaatgaacagcctgagagctgaggacacggctctgttttactgtgcgagaggatacgatatcttgactggttaccccgactactggggccagggaaccctggtcaccgtctctagt

SEQ ID NO: 441 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatacgccatgcactgggtccgccaggctccaggcaaggggctggagtgggtggcagttatatcatatgatggaggaaataaatactatgcagagtccgtgaagggccggttcaccatctccagagacaattccaagaacaccctgtatctgcaaatgaacagcctgagagctgaggacacggctctgttttactgtgcgagaggatacgatatcttgactggttaccccgactactggggccagggaaccctggtcaccgtctctagt

SEQ ID NO: 442 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcattcgatggaggaaataaatactatgcagagtccgtgaagggccggttcaccatctccagagacaattccaagaacaccctgtatctgcaaatgaacagcctgagagctgaggacacggctctgttttactgtgcgagaggatacgatatcttgactggttaccccgactactggggccagggaaccctggtcaccgtctctagt

SEQ ID NO: 443 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatatgatggagccaataaatactatgcagagtccgtgaagggccggttcaccatctccagagacaattccaagaacaccctgtatctgcaaatgaacagcctgagagctgaggacacggctctgttttactgtgcgagaggatacgatatcttgactggttaccccgactactggggccagggaaccctggtcaccgtctctagt

SEQ ID NO: 444 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtaagtacgccatgcactgggtccgccaggctccaggcaaggggctggagtgggtggcagttatatcattcaagggaagcaataaatactatgcagagtccgtgaagggccggttcaccatctccagagacaattccaagaacaccctgtatctgcaaatgaacagcctgagagctgaggacacggctctgttttactgtgcgagaggatacgatctgttgactggttaccccgactactggggccagggaaccctggtcaccgtctctagt

SEQ ID NO: 445 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatacgccatgcactgggtccgccaggctccaggcaaggggctggagtgggtggcagttatatcatatcagggaggaaataaatactatgcagagtccgtgaagggccggttcaccatctccagagacaattccaagaacaccctgtatctgcaaatgaacagcctgagagctgaggacacggctctgttttactgtgcgagaggatacgatctgttgactggttaccccgactactggggccagggaaccctggtcaccgtctctagt

SEQ ID NO: 446 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtcactttgccatgcactgggtccgccaggctccaggcaaggggctggagtgggtggcagttatatcattcagcggaagcaataaatactatgcagagtccgtgaagggccggttcaccatctccagagacaattccaagaacaccctgtatctgcaaatgaacagcctgagagctgaggacacggctctgttttactgtgcgagaggatacgatatgttgactggttaccccgactactggggccagggaaccctggtcaccgtctctagt

SEQ ID NO: 447 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtaagtttgccatgcactgggtccgccaggctccaggcaaggggctggagtgggtggcagttatatcatatcgcggaggaaataaatactatgcagagtccgtgaagggccggttcaccatctccagagacaattccaagaacaccctgtatctgcaaatgaacagcctgagagctgaggacacggctctgttttactgtgcgagaggatacgatctgttgactggttaccccgactactggggccagggaaccctggtcaccgtctctagt

SEQ ID NO: 448 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatacgccatgcactgggtccgccaggctccaggcaaggggctggagtgggtggcagttatatcatatagcggagccaataaatactatgcagagtccgtgaagggccggttcaccatctccagagacaattccaagaacaccctgtatctgcaaatgaacagcctgagagctgaggacacggctctgttttactgtgcgagaggatacgatctgttgagcggttaccccgactactggggccagggaaccctggtcaccgtctctagt

SEQ ID NO: 449 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtcactacgccatgcactgggtccgccaggctccaggcaaggggctggagtgggtggcagttatatcattcaagggagccaataaatactatgcagagtccgtgaagggccggttcaccatctccagagacaattccaagaacaccctgtatctgcaaatgaacagcctgagagctgaggacacggctctgttttactgtgcgagaggatacgatctgttgactggttaccccgactactggggccagggaaccctggtcaccgtctctagt

SEQ ID NO: 450 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtcactacgccatgcactgggtccgccaggctccaggcaaggggctggagtgggtggcagttatatcatatcgcggagccaataaatactatgcagagtccgtgaagggccggttcaccatctccagagacaattccaagaacaccctgtatctgcaaatgaacagcctgagagctgaggacacggctctgttttactgtgcgagaggatacgatctgttgactggttaccccgactactggggccagggaaccctggtcaccgtctctagt

SEQ ID NO: 451 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtcactacgccatgcactgggtccgccaggctccaggcaaggggctggagtgggtggcagttatatcattctacggaagcaataaatactatgcagagtccgtgaagggccggttcaccatctccagagacaattccaagaacaccctgtatctgcaaatgaacagcctgagagctgaggacacggctctgttttactgtgcgagaggatacgatttcttgactggttaccccgactactggggccagggaaccctggtcaccgtctctagt

SEQ ID NO: 452 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtcactttgccatgcactgggtccgccaggctccaggcaaggggctggagtgggtggcagttatatcattcttcggaggaaataaatactatgcagagtccgtgaagggccggttcaccatctccagagacaattccaagaacaccctgtatctgcaaatgaacagcctgagagctgaggacacggctctgttttactgtgcgagaggatacgatttcttgactggttaccccgactactggggccagggaaccctggtcaccgtctctagt

SEQ ID NO: 453 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtcactacgccatgcactgggtccgccaggctccaggcaaggggctggagtgggtggcagttatatcattcatgggaaccaataaatactatgcagagtccgtgaagggccggttcaccatctccagagacaattccaagaacaccctgtatctgcaaatgaacagcctgagagctgaggacacggctctgttttactgtgcgagaggatacgatttcttgactggttaccccgactactggggccagggaaccctggtcaccgtctctagt

SEQ ID NO: 454 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagttactttgccatgcactgggtccgccaggctccaggcaaggggctggagtgggtggcagttatatcacaccgcggaaccaataaatactatgcagagtccgtgaagggccggttcaccatctccagagacaattccaagaacaccctgtatctgcaaatgaacagcctgagagctgaggacacggctctgttttactgtgcgagaggatacgatctgttgagcggttaccccgactactggggccagggaaccctggtcaccgtctctagt

SEQ ID NO: 455 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtgggtgttatcaactatcgtggacatggtaaatactatgcagagtccgtgaagggccggttcaccgtgtccagagacaattccaagaacaccctgtatctgcaaatgaacagcctgagagctgaggacacggctctgttttactgtgcgagaggatacgatgttttgactggttaccccgactactggggccagggaaccctggtcaccgtgtctagt

SEQ ID NO: 456 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtgggtgttatatctttttctggaggttctaaatactatgcagagtccgtgaagggccggttcaccttgtccagagacaattccaagaacaccctgtatctgcaaatgaacagcctgagagctgaggacacggctctgttttactgtgcgagaggatacgatgttttgactggttaccccgactactggggccagggaaccctggtcaccgtgtctagt

SEQ ID NO: 457 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtgggtgttatctcttatactggacagttcaaatactatgcagagtccgtgaagggccggttcaccgtgtccagagacaattccaagaacaccctgtatctgcaaatgaacagcctgagagctgaggacacggctctgttttactgtgcgagaggatacgatgttttgactggttaccccgactactggggccagggaaccctggtcaccgtgtctagt

SEQ ID NO: 458 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtgggtgttatatcttatactggagctcagaaatactatgcagagtccgtgaagggccggttcaccatgtccagagacaattccaagaacaccctgtatctgcaaatgaacagcctgagagctgaggacacggctctgttttactgtgcgagaggatacgatgttttgactggttaccccgactactggggccagggaaccctggtcaccgtgtctagt

SEQ ID NO: 459 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtgggtgttatatcttattctggagcttctaaatactatgcagagtccgtgaagggccggttcaccatgtccagagacaattccaagaacaccctgtatctgcaaatgaacagcctgagagctgaggacacggctctgttttactgtgcgagaggatacgatgttttgactggttaccccgactactggggccagggaaccctggtcaccgtgtctagt

SEQ ID NO: 460 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggctgttatatcttattctggagctttcaaatactatgcagagtccgtgaagggccggttcaccgtgtccagagacaattccaagaacaccctgtatctgcaaatgaacagcctgagagctgaggacacggctctgttttactgtgcgagaggatacgatgttttgactggttaccccgactactggggccagggaaccctggtcaccgtgtctagt

SEQ ID NO: 461 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtgggtgttataacttatactggaggtgctaaatactatgcagagtccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtgtctagt

SEQ ID NO: 462 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtgggtgttatcaactttcagggaactactaaatactatgcagagtccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtgtctagt

SEQ ID NO: 463 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtgggtgttatatcttattctggagatctgaaatactatgcagagtccgtgaagggccggttcaccgtgtccagagacaattccaagaacaccctgtatctgcaaatgaacagcctgagagctgaggacacggctctgttttactgtgcgagaggatacgatgttttgactggttaccccgactactggggccagggaaccctggtcaccgtgtctagt

SEQ ID NO: 464 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtgggtgttatcaactatttcggagacgctaaatactatgcagagtccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtgtctagt

SEQ ID NO: 465 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgagactctcctgtgcagcctctggattcaccttcagtttctacgccatgcactgggtccgccaggctccaggcaaggggctggagtgggtggcagttatctcatctttcggaagtaataaatactatgcagagtccgtgaagggccggttcaccatctccagagacaattccaagaacaccctgtatctgcaaatgaacagcctgagagctgaggacacggctctgttttactgtgcgagaggatacgatctgctgactggttaccccgactactggggccagggaaccctggtcaccgtgtctagt

SEQ ID NO: 466 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagttactacgccatgcactgggtccgccaggctccaggcaaggggctggagtgggtggcagttatctcatactctggaagtaataaatactatgcagagtccgtgaagggccggttcaccatctccagagacaattccaagaacaccctgtatctgcaaatgaacagcctgagagctgaggacacggctctgttttactgtgcgagaggatacgatctgctgactggttaccccgactactggggccagggaaccctggtcaccgtgtctagt

SEQ ID NO: 467 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagttactacgccatgcactgggtccgccaggctccaggcaaggggctggagtgggtggcagttatctcacattacggaactaataaatactatgcagagtccgtgaagggccggttcaccatctccagagacaattccaagaacaccctgtatctgcaaatgaacagcctgagagctgaggacacggctctgttttactgtgcgagaggatacgatcctctgactggttaccccgactactggggccagggaaccctggtcaccgtgtctagt

SEQ ID NO: 468 - DNA, 360 bp, synthetic construct
  misc_feature 1..360: note = Synthetic polynucleotide
  source 1..360: mol_type = other DNA
caggtgcagctggtggagtctgggggaggcgtggtccagcctgggaggtccctgcgactctcctgtgcagcctctggattcaccttcagtcattacgccatgcactgggtccgccaggctccaggcaaggggctggagtgggtggcagttatctcataccagggaagtaataaatactatgcagagtccgtgaagggccggttcaccatctccagagacaattccaagaacaccctgtatctgcaaatgaacagcctgagagctgaggacacggctctgttttactgtgcgagaggatacgatctgctgactggttaccccgactactggggccagggaaccctggtcaccgtgtctagt

SEQ ID NO: 469 - DNA, 370 bp, synthetic construct
  misc_feature 1..370: note = Synthetic polynucleotide
  source 1..370: mol_type = other DNA
caggtacagctgcagcagtcaggtccaggactggtgaagccctcgcagaccctctcactcacctgtgccatctccggggacagtgtctctagcaacagtgctacttggaactggatcaggcagtccccatcgagaggccttgagtggctgggaaggacatattacaggtccaagtggtctaatcattatgcagtatctgtgaaaagtcgaataaccatcaaccccgacacgtccaagagccagttctccctgcagctgaactctgtgactcccgaggacacggctgtgtattactgtgcaagaggaacgtggaaacagctatggttccttgaccactggggccagggaaccctggtcaccgtctctagtg

SEQ ID NO: 470 - DNA, 369 bp, synthetic construct
  misc_feature 1..369: note = Synthetic polynucleotide
  source 1..369: mol_type = other DNA
caggtacagctgcagcagtcaggtccaggactggtgaagccctcgcagaccctctcactcacctgtgccatctccggggacagtgtctctaaccgtctggctacttggaactggatcaggcagtccccatcgagaggccttgagtggctgggaaggacatactacaggggtaaatggaaaaatcattatgcagtatctgtgaaaagtcgaataaccatcaaccccgacacgtccaagagccagttctccctgcagctgaactctgtgactcccgaggacacggctgtgtattactgtgcaagaggaacttggaaccaggactggttcctggaccactggggccagggaaccctggtcaccgtctctagt

SEQ ID NO: 471 - DNA, 369 bp, synthetic construct
  misc_feature 1..369: note = Synthetic polynucleotide
  source 1..369: mol_type = other DNA
caggtacagctgcagcagtcaggtccaggactggtgaagccctcgcagaccctctcactcacctgtgccatctccggggacagtgtctctaaccgtctggctacttggaactggatcaggcagtccccatcgagaggccttgagtggctgggaaggacatactacaggggtaaatggaaaaatcattatgcagtatctgtgaaaagtcgaataaccatcaaccccgacacgtccaagagccagttctccctgcagctgaactctgtgactcccgaggacacggctgtgtattactgtgcaagaggaacttgggaccaggactggttcctggaccactggggccagggaaccctggtcaccgtctctagt

SEQ ID NO: 472 - DNA, 369 bp, synthetic construct
  misc_feature 1..369: note = Synthetic polynucleotide
  source 1..369: mol_type = other DNA
caggtacagctgcagcagtcaggtccaggactggtgaagccctcgcagaccctctcactcacctgtgccatctccggggacagtgtctcttctcgtcaggctacttggaactggatcaggcagtccccatcgagaggccttgagtggctgggaaggacatactacaggggtaaatggaaaaatcattatgcagtatctgtgaaaagtcgaataaccatcaaccccgacacgtccaagagccagttctccctgcagctgaactctgtgactcccgaggacacggctgtgtattactgtgcaagaggaacttgggaacaggactggttcctggaccactggggccagggaaccctggtcaccgtctctagt

SEQ ID NO: 473 - DNA, 369 bp, synthetic construct
  misc_feature 1..369: note = Synthetic polynucleotide
  source 1..369: mol_type = other DNA
caggtacagctgcagcagtcaggtccaggactggtgaagccctcgcagaccctctcactcacctgtgccatctccggggacagtgtctctaacaaacaggctacttggaactggatcaggcagtccccatcgagaggccttgagtggctgggaaggacatactacaggggtaaatggaaaaatcattatgcagtatctgtgaaaagtcgaataaccatcaaccccgacacgtccaagagccagttctccctgcagctgaactctgtgactcccgaggacacggctgtgtattactgtgcaagaggaatgtggaaccagaactggttcctggaccactggggccagggaaccctggtcaccgtctctagt

SEQ ID NO: 474 - DNA, 369 bp, synthetic construct
  misc_feature 1..369: note = Synthetic polynucleotide
  source 1..369: mol_type = other DNA
caggtacagctgcagcagtcaggtccaggactggtgaagccctcgcagaccctctcactcacctgtgccatctccggggacagtgtctctaacaaacaggctacttggaactggatcaggcagtccccatcgagaggccttgagtggctgggaaggacatactacaggggtcagtggaaaaatcattatgcagtatctgtgaaaagtcgaataaccatcaaccccgacacgtccaagagccagttctccctgcagctgaactctgtgactcccgaggacacggctgtgtattactgtgcaagaggaacttggatcggtgactggttcatggaccactggggccagggaaccctggtcaccgtctctagt

SEQ ID NO: 475 - DNA, 369 bp, synthetic construct
  misc_feature 1..369: note = Synthetic polynucleotide
  source 1..369: mol_type = other DNA
caggtacagctgcagcagtcaggtccaggactggtgaagccctcgcagaccctctcactcacctgtgccatctccggggacagtgtctctaacaaacaggctacttggaactggatcaggcagtccccatcgagaggccttgagtggctgggaaggacatactacaggggtaaatggaaaaatcattatgcagtatctgtgaaaagtcgaataaccatcaaccccgacacgtccaagagccagttctccctgcagctgaactctgtgactcccgaggacacggctgtgtattactgtgcaagaggaacttggatccaggactggttcctggaccactggggccagggaaccctggtcaccgtctctagt

SEQ ID NO: 476 - DNA, 369 bp, synthetic construct
  misc_feature 1..369: note = Synthetic polynucleotide
  source 1..369: mol_type = other DNA
caggtacagctgcagcagtcaggtccaggactggtgaagccctcgcagaccctctcactcacctgtgccatctccggggacagtgtctcttctaaccatgctacttggaactggatcaggcagtccccatcgagaggccttgagtggctgggaaggacatactacaggggtaaatggaaaaatcattatgcagtatctgtgaaaagtcgaataaccatcaaccccgacacgtccaagagccagttctccctgcagctgaactctgtgactcccgaggacacggctgtgtattactgtgcaagaggaacttgggaccaggactggttcctggaccactggggccagggaaccctggtcaccgtctctagt

SEQ ID NO: 477 - DNA, 369 bp, synthetic construct
  misc_feature 1..369: note = Synthetic polynucleotide
  source 1..369: mol_type = other DNA
caggtacagctgcagcagtcaggtccaggactggtgaagccctcgcagaccctctcactcacctgtgccatctccggggacagtgtctcttctcgttacgctacttggaactggatcaggcagtccccatcgagaggccttgagtggctgggaaggacatactacaggggtcagtggaaaaatcattatgcagtatctgtgaaaagtcgaataaccatcaaccccgacacgtccaagagccagttctccctgcagctgaactctgtgactcccgaggacacggctgtgtattactgtgcaagaggaatgtggaaccagaactggttcctggaccactggggccagggaaccctggtcaccgtctctagt

SEQ ID NO: 478 - DNA, 369 bp, synthetic construct
  misc_feature 1..369: note = Synthetic polynucleotide
  source 1..369: mol_type = other DNA
caggtacagctgcagcagtcaggtccaggactggtgaagccctcgcagaccctctcactcacctgtgccatctccggggacagtgtctctaacaaacaggctacttggaactggatcaggcagtccccatcgagaggccttgagtggctgggaaggacatactacaggggtaaatggaaaaatcattatgcagtatctgtgaaaagtcgaataaccatcaaccccgacacgtccaagagccagttctccctgcagctgaactctgtgactcccgaggacacggctgtgtattactgtgcaagaggaatgtggtctggtgactggttcctggaccactggggccagggaaccctggtcaccgtctctagt

SEQ ID NO: 479 - DNA, 369 bp, synthetic construct
  misc_feature 1..369: note = Synthetic polynucleotide
  source 1..369: mol_type = other DNA
caggtacagctgcagcagtcaggtccaggactggtgaagccctcgcagaccctctcactcacctgtgccatctccggggacagtgtctcttctcatgttgctacttggaactggatcaggcagtccccatcgagaggccttgagtggctgggaaggacatactacaggggtaaatggaaaaatcattatgcagtatctgtgaaaagtcgaataaccatcaaccccgacacgtccaagagccagttctccctgcagctgaactctgtgactcccgaggacacggctgtgtattactgtgcaagaggaatgtggtctgaagactggttcctggaccactggggccagggaaccctggtcaccgtctctagt

SEQ ID NO: 480 - DNA, 369 bp, synthetic construct
  misc_feature 1..369: note = Synthetic polynucleotide
  source 1..369: mol_type = other DNA
caggtacagctgcagcagtcaggtccaggactggtgaagccctcgcagaccctctcactcacctgtgccatctccggggacagtgtctcttctcgtcaggctacttggaactggatcaggcagtccccatcgagaggccttgagtggctgggaaggacatactacaggggtaaatggaaaaatcattatgcagtatctgtgaaaagtcgaataaccatcaaccccgacacgtccaagagccagttctccctgcagctgaactctgtgactcccgaggacacggctgtgtattactgtgcaagaggaatgtggcagggtaactggttcctggaccactggggccagggaaccctggtcaccgtctctagt

SEQ ID NO: 481 - DNA, 369 bp, synthetic construct
  misc_feature 1..369: note = Synthetic polynucleotide
  source 1..369: mol_type = other DNA
caggtacagctgcagcagtcaggtccaggactggtgaagccctcgcagaccctctcactcacctgtgccatctccggggacagtgtctctaaccatcaggctacttggaactggatcaggcagtccccatcgagaggccttgagtggctgggaaggacatactacaggggtaaatggaaaaatcattatgcagtatctgtgaaaagtcgaataaccatcaaccccgacacgtccaagagccagttctccctgcagctgaactctgtgactcccgaggacacggctgtgtattactgtgcaagaggaacttggatccaggactggttcctggaccactggggccagggaaccctggtcaccgtctctagt

SEQ ID NO: 482 - DNA, 369 bp, synthetic construct
  misc_feature 1..369: note = Synthetic polynucleotide
  source 1..369: mol_type = other DNA
caggtacagctgcagcagtcaggtccaggactggtgaagccctcgcagaccctctcactcacctgtgccatctccggggacagtgtctcttctcgtgacgctacttggaactggatcaggcagtccccatcgagaggccttgagtggctgggaaggacatactacaggggtaaatggaaaaatcattatgcagtatctgtgaaaagtcgaataaccatcaaccccgacacgtccaagagccagttctccctgcagctgaactctgtgactcccgaggacacggctgtgtattactgtgcaagaggacagtggaacgaagactggttcctggaccactggggccagggaaccctggtcaccgtctctagt

SEQ ID NO: 483 - DNA, 369 bp, synthetic construct
  misc_feature 1..369: note = Synthetic polynucleotide
  source 1..369: mol_type = other DNA
caggtacagctgcagcagtcaggtccaggactggtgaagccctcgcagaccctctcactcacctgtgccatctccggggacagtgtctctaaccgtctggctacttggaactggatcaggcagtccccatcgagaggccttgagtggctgggaaggacatactacaggggtaaatggaaaaatcattatgcagtatctgtgaaaagtcgaataaccatcaaccccgacacgtccaagagccagttctccctgcagctgaactctgtgactcccgaggacacggctgtgtattactgtgcaagaggacgttgggaaggtgactggttcttcgaccactggggccagggaaccctggtcaccgtctctagt

SEQ ID NO: 484 - DNA, 369 bp, synthetic construct
  misc_feature 1..369: note = Synthetic polynucleotide
  source 1..369: mol_type = other DNA
caggtacagctgcagcagtcaggtccaggactggtgaagccctcgcagaccctctcactcacctgtgccatctccggggacagtgtctctaacaaacaggctacttggaactggatcaggcagtccccatcgagaggccttgagtggctgggaaggacatacttcaggcgtacttggaaaaatcattatgcagtatctgtgaaaagtcgaataaccatcaaccccgacacgtccaagagccagttctccctgcagctgaactctgtgactcccgaggacacggctgtgtattactgtgcaagaggaatgtggtctgaagactggttcctggaccactggggccagggaaccctggtcaccgtctctagt

SEQ ID NO: 485 - DNA, 369 bp, synthetic construct
  misc_feature 1..369: note = Synthetic polynucleotide
  source 1..369: mol_type = other DNA
caggtacagctgcagcagtcaggtccaggactggtgaagccctcgcagaccctctcactcacctgtgccatctccggggacagtgtctctaaccgtctggctacttggaactggatcaggcagtccccatcgagaggccttgagtggctgggaaggacatactacaggggtaaatggaaaaatcattatgcagtatctgtgaaaagtcgaataaccatcaaccccgacacgtccaagagccagttctccctgcagctgaactctgtgactcccgaggacacggctgtgtattactgtgcaagaggagtttggatcggtaactggttcctggaccactggggccagggaaccctggtcaccgtctctagt

SEQ ID NO: 486 - protein, 22 aa, synthetic construct
  REGION 1..22: note = Synthetic polypeptide
MDMRVPAQLLGLLLLWLRGARC

SEQ ID NO: 487 - protein, 19 aa, synthetic construct
  REGION 1..19: note = Synthetic polypeptide
MAWALLLLTLLTQGTGSWA

SEQ ID NO: 488 - protein, 19 aa, synthetic construct
  REGION 1..19: note = Synthetic polypeptide
MTCSPLLLTLLIHCTGSWA

SEQ ID NO: 489 - protein, 20 aa, synthetic construct
  REGION 1..20: note = Synthetic polypeptide
MEAPAQLLFLLLLWLPDTTG

SEQ ID NO: 490 - protein, 19 aa, synthetic construct
  REGION 1..19: note = Synthetic polypeptide
MEWTWRVLFLVAAATGAHS

SEQ ID NO: 491 - protein, 20 aa, synthetic construct
  REGION 1..20: note = Synthetic polypeptide
METPAQLLFLLLLWLPDTTG

SEQ ID NO: 492 - protein, 20 aa, synthetic construct
  REGION 1..20: note = Synthetic polypeptide
METPAQLLFLLLLWLPDTTG

SEQ ID NO: 493 - protein, 19 aa, synthetic construct
  REGION 1..19: note = Synthetic polypeptide
MKHLWFFLLLVAAPRWVLS

SEQ ID NO: 494 - protein, 19 aa, synthetic construct
  REGION 1..19: note = Synthetic polypeptide
MEWSWVFLFFLSVTTGVHS

SEQ ID NO: 495 - protein, 22 aa, synthetic construct
  REGION 1..22: note = Synthetic polypeptide
MDIRAPTQLLGLLLLWLPGAKC

SEQ ID NO: 496 - protein, 22 aa, synthetic construct
  REGION 1..22: note = Synthetic polypeptide
MDIRAPTQLLGLLLLWLPGARC

SEQ ID NO: 497 - protein, 22 aa, synthetic construct
  REGION 1..22: note = Synthetic polypeptide
MDTRAPTQLLGLLLLWLPGATF

SEQ ID NO: 498 - protein, 22 aa, synthetic construct
  REGION 1..22: note = Synthetic polypeptide
MDTRAPTQLLGLLLLWLPGARC

SEQ ID NO: 499 - protein, 19 aa, synthetic construct
  REGION 1..19: note = Synthetic polypeptide
METGLRWLLLVAVLKGVQC

SEQ ID NO: 500 - protein, 21 aa, synthetic construct
  REGION 1..21: note = Synthetic polypeptide
METGLRWLLLVAVLKGVQCQE

SEQ ID NO: 501 - protein, 22 aa, synthetic construct
  REGION 1..22: note = Synthetic polypeptide
MDMRAPTQLLGLLLLWLPGARC

SEQ ID NO: 502 - protein, 110 aa, synthetic construct
  REGION 1..110: note = Synthetic polypeptide
QSVLTQPPSVSAAPGQKVTISCSGSSSNIGNNYVSWYQQLPGTAPKLLIYDNNKRPSGIPDRFSGSKSGTSTTLGITGLQTGDEADYYCGTWDSRLSAVVFGGGTKLTVL

SEQ ID NO: 503 - protein, 130 aa, synthetic construct [sequence omitted]

SEQ ID NO: 504 - protein, 214 aa, synthetic construct
  REGION 1..214: note = Synthetic polypeptide
EIVLTQSPATLSLSPGERATLSCRASQSVGRSLHWYQQKPGQAPRLLIKYASQSLSGIPARFSGSGSGTDFTLTISSLEPEDFAVYYCHQSSRLPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 505 - protein, 214 aa, synthetic construct; identical to SEQ ID NO: 3
  REGION 1..214: note = Synthetic polypeptide

SEQ ID NO: 506 - protein, 214 aa, synthetic construct
  REGION 1..214: note = Synthetic polypeptide
EIVLTQSPATLSLSPGERATLSCRASKSVGRSLHWYQQKPGQAPRLLIKYASQSLSGIPARFSGSGSGTDFTLTISSLEPEDFAVYYCHQSSRLPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 507 - protein, 214 aa, synthetic construct
  REGION 1..214: note = Synthetic polypeptide
EIVLTQSPATLSLSPGERATLSCRASKSVGWSLHWYQQKPGQAPRLLIKYASQSLSGIPARFSGSGSGTDFTLTISSLEPEDFAVYYCHQSSRLPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 508 - protein, 142 aa, synthetic construct
  REGION 1..142: note = Synthetic polypeptide
LTISSLEPEDFAVYYCHQSSRLPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 509 - protein, 214 aa, synthetic construct
  REGION 1..214: note = Synthetic polypeptide
DIQMTQSPSSLSASVGDRITITCRASQSISRYLNWYQQKPGKAPKLLIYAASSLQSGIPSRFSGSGSGTDFTLTINSLQPEDFATYFCQQSYSPPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 510 - protein, 214 aa, synthetic construct
  REGION 1..214: note = Synthetic polypeptide
DIQMTQSPSSLSASVGDRITITCRASQMIARYLNWYQQKPGKAPKLLIYASYNLQSGIPSRFSGSGSGTDFTLTINSLQPEDFATYFCQQAIINPYTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 511 - protein, 214 aa, synthetic construct
  REGION 1..214: note = Synthetic polypeptide
DIQMTQSPSSLSASVGDRITITCRASQKIARYLVWYQQKPGKAPKLLIYAANMLQSGIPSRFSGSGSGTDFTLTINSLQPEDFATYFCQQSIQQPYTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 512 - protein, 214 aa, synthetic construct
  REGION 1..214: note = Synthetic polypeptide
DIQMTQSPSSLSASVGDRITITCRASQSISRYLNWYQQKPGKAPKLLIYAGRILQSGIPSRFSGSGSGTDFTLTINSLQPEDFATYFCQQAIINPYTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 513 - protein, 214 aa, synthetic construct
  REGION 1..214: note = Synthetic polypeptide
DIQMTQSPSSLSASVGDRITITCRASQSISRYLNWYQQKPGKAPKLLIYASYNLQSGIPSRFSGSGSGTDFTLTINSLQPEDFATYFCQQSIQQPYTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 514 - protein, 214 aa, synthetic construct
  REGION 1..214: note = Synthetic polypeptide
DIQMTQSPSSLSASVGDRITITCRASQYIVRYLNWYQQKPGKAPKLLIYASYNLQSGIPSRFSGSGSGTDFTLTINSLQPEDFATYFCQQAIMAPYTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 515 - protein, 214 aa, synthetic construct
  REGION 1..214: note = Synthetic polypeptide
DIQMTQSPSSLSASVGDRITITCRASQSISRYLNWYQQKPGKAPKLLIFAGQRLQSGIPSRFSGSGSGTDFTLTINSLQPEDFATYFCQQAIGMPYTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 516 - protein, 214 aa, synthetic construct
  REGION 1..214: note = Synthetic polypeptide
DIQMTQSPSSLSASVGDRITITCRASQYIVRYLNWYQQKPGKAPKLLIYAANMLQSGIPSRFSGSGSGTDFTLTINSLQPEDFATYFCQQAINQPYTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 517 - protein, 214 aa, synthetic construct
  REGION 1..214: note = Synthetic polypeptide
DIQMTQSPSSLSASVGDRITITCRASQPISRYLSWYQQKPGKAPKLLIFAGQRLQSGIPSRFSGSGSGTDFTLTINSLQPEDFATYFCQQAISIPYTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 518 - protein, 214 aa, synthetic construct
  REGION 1..214: note = Synthetic polypeptide
DIQMTQSPSSLSASVGDRITITCRASQQIARYLNWYQQKPGKAPKLLIYASYNLQSGIPSRFSGSGSGTDFTLTINSLQPEDFATYFCQQAIIQPYTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 519 - protein, 450 aa, synthetic construct
  REGION 1..450: note = Synthetic polypeptide
QVQLVESGGGVVQPGRSLRLSCAASGFTFSRFAMHWVRQAPGKGLEWVAVISYDGGNKYYAESVKGRFTISRDNSKNTLYLQMNSLRAEDTALFYCARGYDVLTGYPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 520 - protein, 450 aa, synthetic construct
  REGION 1..450: note = Synthetic polypeptide
QVQLVESGAEVVKPGASVKVSCKASGFTFSRFAMHWVRQAPGQGLEWMGVISYDGGNKYYAESVKGRVTMTRDTSTSTLYMELSSLRSEDTAVYYCARGYDVLTGYPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 521 - protein, 450 aa, synthetic construct
  REGION 1..450: note = Synthetic polypeptide
QVQLVESGGGVVQPGRSLRLSCAASGFTFSRFAMHWVRQAPGKGLEWVAVISYIGGNKYYAESVKGRFTISRDNSKNTLYLQMNSLRAEDTALFYCARGYDVLTGYPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 522 - protein, 450 aa, synthetic construct
  REGION 1..450: note = Synthetic polypeptide
QVQLVESGGGVVQPGRSLRLSCAASGFTFSRFAMHWVRQAPGKGLEWVAVISYQGRNKYYAESVKGRFTISRDNSKNTLYLQMNSLRAEDTALFYCARGYDVLTGYPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 523 - protein, 450 aa, synthetic construct
  REGION 1..450: note = Synthetic polypeptide
QVQLVESGGGVVQPGRSLRLSCAASGFTFSRFAMHWVRQAPGKGLEWVAVISYQGNNKYYARSVKGRFTISRDNSKNTLYLQMNSLRAEDTALFYCARGYDVLTGYPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 524 - protein, 450 aa, synthetic construct
  REGION 1..450: note = Synthetic polypeptide
QVQLVESGGGVVQPGRSLRLSCAASGFTFSKFAMHWVRQAPGKGLEWVAVISYRGGNKYYAESVKGRFTISRDNSKNTLYLQMNSLRAEDTALFYCARGYDLLTGYPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 525 - protein, 450 aa, synthetic construct
  REGION 1..450: note = Synthetic polypeptide
QVQLVESGGGVVQPGRSLRLSCAASGFTFSRFAMHWVRQAPGKGLEWVAVISYIGGNKYYAESVKGRFTISRDNSKNTLYLQMNSLRAEDTALFYCARGYDILTGYPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 526 - protein, 450 aa, synthetic construct
  REGION 1..450: note = Synthetic polypeptide
QVQLVESGGGVVQPGRSLRLSCAASGFTFSRFAMHWVRQAPGKGLEWVAVISYNGRNKYYARSVKGRFTISRDNSKNTLYLQMNSLRAEDTALFYCARGYDILTGYPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 527 - protein, 450 aa, synthetic construct
  REGION 1..450: note = Synthetic polypeptide
QVQLVESGGGVVQPGRSLRLSCAASGFTFSRFAMHWVRQAPGKGLEWVGVINYRGHGKYYAESVKGRFTVSRDNSKNTLYLQMNSLRAEDTALFYCARGYDVLTGYPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 528 - protein, 450 aa, synthetic construct
  REGION 1..450: note = Synthetic polypeptide
QVQLVESGGGVVQPGRSLRLSCAASGFTFSRFAMHWVRQAPGKGLEWVGVISFSGGSKYYAESVKGRFTLSRDNSKNTLYLQMNSLRAEDTALFYCARGYDVLTGYPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 529 - protein, 450 aa, synthetic construct
  REGION 1..450: note = Synthetic polypeptide
QVQLVESGGGVVQPGRSLRLSCAASGFTFSRFAMHWVRQAPGKGLEWVGVISYTGQFKYYAESVKGRFTVSRDNSKNTLYLQMNSLRAEDTALFYCARGYDVLTGYPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 530 - protein, 453 aa, synthetic construct
  REGION 1..453: note = Synthetic polypeptide
QVQLQQSGPGLVKPSQTLSLTCAISGDSVSSNSATWNWIRQSPSRGLEWLGRTYYRSKWSNHYAVSVKSRITINPDTSKSQFSLQLNSVTPEDTAVYYCARGTWKQLWFLDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 531 - protein, 453 aa, synthetic construct
  REGION 1..453: note = Synthetic polypeptide
QVQLQQSGPGLVKPSQTLSLTCAISGDSVSNRLATWNWIRQSPSRGLEWLGRTYYRGKWKNHYAVSVKSRITINPDTSKSQFSLQLNSVTPEDTAVYYCARGVWIGNWFLDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 532 - protein, 453 aa, synthetic construct
  REGION 1..453: note = Synthetic polypeptide
QVQLQQSGPGLVKPSQTLSLTCAISGDSVSNKQATWNWIRQSPSRGLEWLGRTYYRGKWKNHYAVSVKSRITINPDTSKSQFSLQLNSVTPEDTAVYYCARGMWNQNWFLDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 533 - protein, 453 aa, synthetic construct
  REGION 1..453: note = Synthetic polypeptide
QVQLQQSGPGLVKPSQTLSLTCAISGDSVSSRQATWNWIRQSPSRGLEWLGRTYYRGKWKNHYAVSVKSRITINPDTSKSQFSLQLNSVTPEDTAVYYCARGMWQGNWFLDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 534 - protein, 453 aa, synthetic construct
  REGION 1..453: note = Synthetic polypeptide
QVQLQQSGPGLVKPSQTLSLTCAISGDSVSNRLATWNWIRQSPSRGLEWLGRTYYRGKWKNHYAVSVKSRITINPDTSKSQFSLQLNSVTPEDTAVYYCARGRWEGDWFFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 535 - protein, 453 aa, synthetic construct
  REGION 1..453: note = Synthetic polypeptide
QVQLQQSGPGLVKPSQTLSLTCAISGDSVSNRLATWNWIRQSPSRGLEWLGRTYYRGKWKNHYAVSVKSRITINPDTSKSQFSLQLNSVTPEDTAVYYCARGTWNQDWFLDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 536 - protein, 453 aa, synthetic construct
  REGION 1..453: note = Synthetic polypeptide
QVQLQQSGPGLVKPSQTLSLTCAISGDSVSSRYATWNWIRQSPSRGLEWLGRTYYRGQWKNHYAVSVKSRITINPDTSKSQFSLQLNSVTPEDTAVYYCARGMWNQNWFLDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 537 - DNA, 645 bp, synthetic construct
  misc_feature 1..645: note = Synthetic polynucleotide
  source 1..645: mol_type = other DNA
gagatcgtacttactcagtcacccgccacattgtccctgagcccgggtgaacgggcgaccctcagctgccgagcatcccagtccgtcggacgatcattgcactggtaccaacaaaaaccgggccaggcccccagacttctgatcaagtatgcgtcacagagcttgtcgggtattcccgctcgcttttcggggtcgggatccgggacagatttcacgctcacaatctcctcgctggaacccgaggacttcgcggtctactattgtcatcagtcatcgaggttgcctttcacgtttggaccagggaccaaggtggacattaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 538 - DNA, 642 bp, synthetic construct
  misc_feature 1..642: note = Synthetic polynucleotide
  source 1..642: mol_type = other DNA
gatatccagctcactcaatcgccatcatttctctccgcttcggtaggcgaccgggtcacgatcacatgcagggcgtcgcaaagcattgggaggtcgttgcattggtatcagcagaaacccggaaaggccccgaaacttctgatcaaatacgcatcacaaagcttgagcggtgtgccgtcgcgcttctccggttccggaagcggaacggaattcacgcttacaatctcctcactgcagcccgaggatttcgcgacctattactgtcaccagtcatccagactcccgtttacttttggccctgggaccaaggtggacattaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 539 - DNA, 642 bp, synthetic construct
  misc_feature 1..642: note = Synthetic polynucleotide
  source 1..642: mol_type = other DNA
gagatcgtacttactcagtcacccgccacattgtccctgagcccgggtgaacgggcgaccctcagctgccgagcatccaagtccgtcggacgatcattgcactggtaccaacaaaaaccgggccaggcccccagacttctgatcaagtatgcgtcacagagcttgtcgggtattcccgctcgcttttcggggtcgggatccgggacagatttcacgctcacaatctcctcgctggaacccgaggacttcgcggtctactattgtcatcagtcatcgaggttgcctttcacgtttggaccagggaccaaggtggacattaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 540 - DNA, 642 bp, synthetic construct
  misc_feature 1..642: note = Synthetic polynucleotide
  source 1..642: mol_type = other DNA
gagatcgtacttactcagtcacccgccacattgtccctgagcccgggtgaacgggcgaccctcagctgccgagcatccaaatccgtcgggtggagcttgcactggtaccaacaaaaaccgggccaggcccccagacttctgatcaagtatgcgtcacagagcttgtcgggtattcccgctcgcttttcggggtcgggatccgggacagatttcacgctcacaatctcctcgctggaacccgaggacttcgcggtctactattgtcatcagtcatcgaggttgcctttcacgtttggaccagggaccaaggtggacattaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 541 - DNA, 642 bp, synthetic construct
  misc_feature 1..642: note = Synthetic polynucleotide
  source 1..642: mol_type = other DNA
gagatcgtacttactcagtcacccgccacattgtccctgagcccgggtgaacgggcgaccctcagctgccgagcatccaaatccgtcgggtacagcttgcactggtaccaacaaaaaccgggccaggcccccagacttctgatcaagtatgcgtcacagagcttgtcgggtattcccgctcgcttttcggggtcgggatccgggacagatttcacgctcacaatctcctcgctggaacccgaggacttcgcggtctactattgtcatcagtcatcgaggttgcctttcacgtttggaccagggaccaaggtggacattaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 542 - DNA, 642 bp, synthetic construct
  misc_feature 1..642: note = Synthetic polynucleotide
  source 1..642: mol_type = other DNA
gacatccagatgacccagtctccatcctccctgtctgcatctgtaggagacagaatcaccatcacttgccgggcaagtcagagcattagcaggtatttaaattggtatcaacagaaaccagggaaagcccctaaactcctgatctatgctgcatccagtttgcaaagtgggatcccatcaaggttcagcggcagtggatctgggacagatttcactctcaccatcaacagtctgcaacctgaagattttgcaacttacttctgtcaacagagttacagtcccccattcactttcggccctgggaccaaagtggatatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 543 - DNA, 642 bp, synthetic construct
  misc_feature 1..642: note = Synthetic polynucleotide
  source 1..642: mol_type = other DNA
gacatccagatgacccagtctccatcctccctgtctgcatctgtaggagacagaatcaccatcacttgccgggcaagtcagatgattgctcgttacttaaactggtatcaacagaaaccagggaaagcccctaaactcctgatctacgcttcttacaacttgcaaagtgggatcccatcaaggttcagcggcagtggatctgggacagatttcactctcaccatcaacagtctgcaacctgaagattttgcaacttacttctgtcaacaggctatcatcaacccatacactttcggccctgggaccaaagtggatatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 544 - DNA, 642 bp, synthetic construct
  misc_feature 1..642: note = Synthetic polynucleotide
  source 1..642: mol_type = other DNA
gacatccagatgacccagtctccatcctccctgtctgcatctgtaggagacagaatcaccatcacttgccgggcaagtcagaaaattgctcgttacttagtttggtatcaacagaaaccagggaaagcccctaaactcctgatctacgctgctaacatgttgcaaagtgggatcccatcaaggttcagcggcagtggatctgggacagatttcactctcaccatcaacagtctgcaacctgaagattttgcaacttacttctgtcaacagtctatccagcagccatacactttcggccctgggaccaaagtggatatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 545 - DNA, 642 bp, synthetic construct
  misc_feature 1..642: note = Synthetic polynucleotide
  source 1..642: mol_type = other DNA
gacatccagatgacccagtctccatcctccctgtctgcatctgtaggagacagaatcaccatcacttgccgggcaagtcagagcattagcaggtatttaaattggtatcaacagaaaccagggaaagcccctaaactcctgatctacgctggtcgtatcttgcaaagtgggatcccatcaaggttcagcggcagtggatctgggacagatttcactctcaccatcaacagtctgcaacctgaagattttgcaacttacttctgtcaacaggctatcatcaacccatacactttcggccctgggaccaaagtggatatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 546 - DNA, 642 bp, synthetic construct
  misc_feature 1..642: note = Synthetic polynucleotide
  source 1..642: mol_type = other DNA
gacatccagatgacccagtctccatcctccctgtctgcatctgtaggagacagaatcaccatcacttgccgggcaagtcagagcattagcaggtatttaaattggtatcaacagaaaccagggaaagcccctaaactcctgatctacgcttcttacaacttgcaaagtgggatcccatcaaggttcagcggcagtggatctgggacagatttcactctcaccatcaacagtctgcaacctgaagattttgcaacttacttctgtcaacagtctatccagcagccatacactttcggccctgggaccaaagtggatatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 547 - DNA, 642 bp, synthetic construct
  misc_feature 1..642: note = Synthetic polynucleotide
  source 1..642: mol_type = other DNA
gacatccagatgacccagtctccatcctccctgtctgcatctgtaggagacagaatcaccatcacttgccgggcaagtcagtacattgttcgttacttaaactggtatcaacagaaaccagggaaagcccctaaactcctgatctacgcttcttacaacttgcaaagtgggatcccatcaaggttcagcggcagtggatctgggacagatttcactctcaccatcaacagtctgcaacctgaagattttgcaacttacttctgtcaacaggctatcatggctccatacactttcggccctgggaccaaagtggatatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 548 - DNA, 642 bp, synthetic construct
  misc_feature 1..642: note = Synthetic polynucleotide
  source 1..642: mol_type = other DNA
gacatccagatgacccagtctccatcctccctgtctgcatctgtaggagacagaatcaccatcacttgccgggcaagtcagagcattagcaggtatttaaattggtatcaacagaaaccagggaaagcccctaaactcctgatcttcgctggtcagcgtttgcaaagtgggatcccatcaaggttcagcggcagtggatctgggacagatttcactctcaccatcaacagtctgcaacctgaagattttgcaacttacttctgtcaacaggctatcggtatgccatacactttcggccctgggaccaaagtggatatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 549 - DNA, 642 bp, synthetic construct
  misc_feature 1..642: note = Synthetic polynucleotide
  source 1..642: mol_type = other DNA
gacatccagatgacccagtctccatcctccctgtctgcatctgtaggagacagaatcaccatcacttgccgggcaagtcagtacattgttcgttacttaaactggtatcaacagaaaccagggaaagcccctaaactcctgatctacgctgctaacatgttgcaaagtgggatcccatcaaggttcagcggcagtggatctgggacagatttcactctcaccatcaacagtctgcaacctgaagattttgcaacttacttctgtcaacaggctatcaaccagccatacactttcggccctgggaccaaagtggatatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 550 - DNA, 642 bp, synthetic construct
  misc_feature 1..642: note = Synthetic polynucleotide
  source 1..642: mol_type = other DNA
gacatccagatgacccagtctccatcctccctgtctgcatctgtaggagacagaatcaccatcacttgccgggcaagtcagccgatttctcgttacttatcttggtatcaacagaaaccagggaaagcccctaaactcctgatcttcgctggtcagcgtttgcaaagtgggatcccatcaaggttcagcggcagtggatctgggacagatttcactctcaccatcaacagtctgcaacctgaagattttgcaacttacttctgtcaacaggctatctctatcccatacactttcggccctgggaccaaagtggatatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 551 - DNA, 642 bp, synthetic construct
  misc_feature 1..642: note = Synthetic polynucleotide
  source 1..642: mol_type = other DNA
gacatccagatgacccagtctccatcctccctgtctgcatctgtaggagacagaatcaccatcacttgccgggcaagtcagcagattgctcgttacttaaactggtatcaacagaaaccagggaaagcccctaaactcctgatctacgcttcttacaacttgcaaagtgggatcccatcaaggttcagcggcagtggatctgggacagatttcactctcaccatcaacagtctgcaacctgaagattttgcaacttacttctgtcaacaggctatcatccagccatacactttcggccctgggaccaaagtggatatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 552 - DNA, 1350 bp, synthetic construct
  misc_feature 1..1350: note = Synthetic polynucleotide
  source 1..1350: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatatgatggaggaaataaatactatgcagagtccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagtgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgtgcgaggagcagtacggcagcacgtaccgttgcgtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 553 - DNA, 1350 bp, synthetic construct
  misc_feature 1..1350: note = Synthetic polynucleotide
  source 1..1350: mol_type = other DNA
caagttcagttggtggagtctggagccgaagtagtaaagccaggagcttcagtgaaagtctcttgtaaagcaagtggattcacgtttagccgctttgccatgcattgggtgcggcaagctcccggtcaggggttggagtggatgggagttattagctatgacgggggcaataagtactacgccgagtctgttaagggtcgggtcacaatgacacgggacacctcaaccagtacactctatatggaactgtctagcctgagatccgaggacaccgctgtgtattattgcgctagggggtacgatgtattgacgggttatcctgattactgggggcaggggacactcgtaaccgtctctagtgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacggcagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 554 - DNA, 1350 bp, synthetic construct
  misc_feature 1..1350: note = Synthetic polynucleotide
  source 1..1350: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatatatcggaggaaataaatactatgcagagtccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagtgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgtgcgaggagcagtacggcagcacgtaccgttgcgtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 555 - DNA, 1350 bp, synthetic construct
  misc_feature 1..1350: note = Synthetic polynucleotide
  source 1..1350: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatatcagggacgcaataaatactatgcagagtccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagtgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgtgcgaggagcagtacggcagcacgtaccgttgcgtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 556 - DNA, 1350 bp, synthetic construct
  misc_feature 1..1350: note = Synthetic polynucleotide
  source 1..1350: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatatcagggaaacaataaatactatgcacgctccgtgaagggccggttcaccatctccagagacaattccaagaacaccctgtatctgcaaatgaacagcctgagagctgaggacacggctctgttttactgtgcgagaggatacgatgttttgactggttaccccgactactggggccagggaaccctggtcaccgtctctagtgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgtgcgaggagcagtacggcagcacgtaccgttgcgtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 557 - DNA, 1350 bp, synthetic construct
  misc_feature 1..1350: note = Synthetic polynucleotide
  source 1..1350: mol_type = other DNA
caggtgcagctggtggagtctgggggaggcgtggtccagcctgggaggtccctgcgactctcctgtgcagcctctggattcaccttcagtaagtttgccatgcactgggtccgccaggctccaggcaaggggctggagtgggtggcagttatatcatatcgcggaggaaataaatactatgcagagtccgtgaagggccggttcaccatctccagagacaattccaagaacaccctgtatctgcaaatgaacagcctgagagctgaggacacggctctgttttactgtgcgagaggatacgatctgttgactggttaccccgactactggggccagggaaccctggtcaccgtctctagtgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgtgcgaggagcagtacggcagcacgtaccgttgcgtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 558 - DNA, 1350 bp, synthetic construct
  misc_feature 1..1350: note = Synthetic polynucleotide
  source 1..1350: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatatatcggaggaaataaatactatgcagagtccgtgaagggccggttcaccatctccagagacaattccaagaacaccctgtatctgcaaatgaacagcctgagagctgaggacacggctctgttttactgtgcgagaggatacgatatcttgactggttaccccgactactggggccagggaaccctggtcaccgtctctagtgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgtgcgaggagcagtacggcagcacgtaccgttgcgtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 559 - DNA, 1350 bp, synthetic construct
  misc_feature 1..1350: note = Synthetic polynucleotide
  source 1..1350: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtggcagttatatcatataacggacgcaataaatactatgcacgctccgtgaagggccggttcaccatctccagagacaattccaagaacaccctgtatctgcaaatgaacagcctgagagctgaggacacggctctgttttactgtgcgagaggatacgatatcttgactggttaccccgactactggggccagggaaccctggtcaccgtctctagtgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgtgcgaggagcagtacggcagcacgtaccgttgcgtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 560 - DNA, 1350 bp, synthetic construct
  misc_feature 1..1350: note = Synthetic polynucleotide
  source 1..1350: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtgggtgttatcaactatcgtggacatggtaaatactatgcagagtccgtgaagggccggttcaccgtgtccagagacaattccaagaacaccctgtatctgcaaatgaacagcctgagagctgaggacacggctctgttttactgtgcgagaggatacgatgttttgactggttaccccgactactggggccagggaaccctggtcaccgtgtctagtgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgtgcgaggagcagtacggcagcacgtaccgttgcgtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 561 - DNA, 1350 bp, synthetic construct
  misc_feature 1..1350: note = Synthetic polynucleotide
  source 1..1350: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtgggtgttatatctttttctggaggttctaaatactatgcagagtccgtgaagggccggttcaccttgtccagagacaattccaagaacaccctgtatctgcaaatgaacagcctgagagctgaggacacggctctgttttactgtgcgagaggatacgatgttttgactggttaccccgactactggggccagggaaccctggtcaccgtgtctagtgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgtgcgaggagcagtacggcagcacgtaccgttgcgtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 562 - DNA, 1350 bp, synthetic construct
  misc_feature 1..1350: note = Synthetic polynucleotide
  source 1..1350: mol_type = other DNA
caggtgcagctggtggagtctgggggaggcgtggtccagcctgggaggtccctgcgactctcctgtgcagcctctggattcaccttcagtagatttgccatgcactgggtccgccaggctccaggcaaggggctggagtgggtgggtgttatctcttatactggacagttcaaatactatgcagagtccgtgaagggccggttcaccgtgtccagagacaattccaagaacaccctgtatctgcaaatgaacagcctgagagctgaggacacggctctgttttactgtgcgagaggatacgatgttttgactggttaccccgactactggggccagggaaccctggtcaccgtgtctagtgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgtgcgaggagcagtacggcagcacgtaccgttgcgtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 563 - DNA, 1359 bp, synthetic construct
  misc_feature 1..1359: note = Synthetic polynucleotide
  source 1..1359: mol_type = other DNA
caggtacagctgcagcagtcaggtccaggactggtgaagccctcgcagaccctctcactcacctgtgccatctccggggacagtgtctctagcaacagtgctacttggaactggatcaggcagtccccatcgagaggccttgagtggctgggaaggacatattacaggtccaagtggtctaatcattatgcagtatctgtgaaaagtcgaatcaccatcaaccccgacacgtccaagagccagttctccctgcagctgaactctgtgactcccgaggacacggctgtgtattactgtgcaagaggaacgtggaaacagctatggttccttgaccactggggccagggaaccctggtcaccgtgtctagtgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgtgcgaggagcagtacggcagcacgtaccgttgcgtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggcaaa

SEQ ID NO: 564 - DNA, 1359 bp, synthetic construct
  misc_feature 1..1359: note = Synthetic polynucleotide
  source 1..1359: mol_type = other DNA
caggtacagctgcagcagtcaggtccaggactggtgaagccctcgcagaccctctcactcacctgtgccatctccggggacagtgtctctaaccgtctggctacttggaactggatcaggcagtccccatcgagaggccttgagtggctgggaaggacatactacaggggtaaatggaaaaatcattatgcagtatctgtgaaaagtcgaataaccatcaaccccgacacgtccaagagccagttctccctgcagctgaactctgtgactcccgaggacacggctgtgtattactgtgcaagaggagtttggatcggtaactggttcctggaccactggggccagggaaccctggtcaccgtgtcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgtgcgaggagcagtacggcagcacgtaccgttgcgtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 565 - DNA, 1359 bp, synthetic construct
  misc_feature 1..1359: note = Synthetic polynucleotide
  source 1..1359: mol_type = other DNA
caggtacagctgcagcagtcaggtccaggactggtgaagccctcgcagaccctctcactcacctgtgccatctccggggacagtgtctctaacaaacaggctacttggaactggatcaggcagtccccatcgagaggccttgagtggctgggaaggacatactacaggggtaaatggaaaaatcattatgcagtatctgtgaaaagtcgaataaccatcaaccccgacacgtccaagagccagttctccctgcagctgaactctgtgactcccgaggacacggctgtgtattactgtgcaagaggaatgtggaaccagaactggttcctggaccactggggccagggaaccctggtcaccgtgtcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgtgcgaggagcagtacggcagcacgtaccgttgcgtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 566 - DNA, 1359 bp, synthetic construct
  misc_feature 1..1359: note = Synthetic polynucleotide
  source 1..1359: mol_type = other DNA
caggtacagctgcagcagtcaggtccaggactggtgaagccctcgcagaccctctcactcacctgtgccatctccggggacagtgtctcttctcgtcaggctacttggaactggatcaggcagtccccatcgagaggccttgagtggctgggaaggacatactacaggggtaaatggaaaaatcattatgcagtatctgtgaaaagtcgaataaccatcaaccccgacacgtccaagagccagttctccctgcagctgaactctgtgactcccgaggacacggctgtgtattactgtgcaagaggaatgtggcagggtaactggttcctggaccactggggccagggaaccctggtcaccgtgtcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgtgcgaggagcagtacggcagcacgtaccgttgcgtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 567 - DNA, 1359 bp, synthetic construct
  misc_feature 1..1359: note = Synthetic polynucleotide
  source 1..1359: mol_type = other DNA
caggtacagctgcagcagtcaggtccaggactggtgaagccctcgcagaccctctcactcacctgtgccatctccggggacagtgtctctaaccgtctggctacttggaactggatcaggcagtccccatcgagaggccttgagtggctgggaaggacatactacaggggtaaatggaaaaatcattatgcagtatctgtgaaaagtcgaataaccatcaaccccgacacgtccaagagccagttctccctgcagctgaactctgtgactcccgaggacacggctgtgtattactgtgcaagaggacgttgggaaggtgactggttcttcgaccactggggccagggaaccctggtcaccgtgtcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgtgcgaggagcagtacggcagcacgtaccgttgcgtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 568 - DNA, 1359 bp, synthetic construct
  misc_feature 1..1359: note = Synthetic polynucleotide
  source 1..1359: mol_type = other DNA
caggtacagctgcagcagtcaggtccaggactggtgaagccctcgcagaccctctcactcacctgtgccatctccggggacagtgtctctaaccgtctggctacttggaactggatcaggcagtccccatcgagaggccttgagtggctgggaaggacatactacaggggtaaatggaaaaatcattatgcagtatctgtgaaaagtcgaataaccatcaaccccgacacgtccaagagccagttctccctgcagctgaactctgtgactcccgaggacacggctgtgtattactgtgcaagaggaacttggaaccaggactggttcctggaccactggggccagggaaccctggtcaccgtgtcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgtgcgaggagcagtacggcagcacgtaccgttgcgtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 569 - DNA, 1359 bp, synthetic construct
  misc_feature 1..1359: note = Synthetic polynucleotide
  source 1..1359: mol_type = other DNA
caggtacagctgcagcagtcaggtccaggactggtgaagccctcgcagaccctctcactcacctgtgccatctccggggacagtgtctcttctcgttacgctacttggaactggatcaggcagtccccatcgagaggccttgagtggctgggaaggacatactacaggggtcagtggaaaaatcattatgcagtatctgtgaaaagtcgaataaccatcaaccccgacacgtccaagagccagttctccctgcagctgaactctgtgactcccgaggacacggctgtgtattactgtgcaagaggaatgtggaaccagaactggttcctggaccactggggccagggaaccctggtcaccgtgtcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgtgcgaggagcagtacggcagcacgtaccgttgcgtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

(19) United States
(12) Patent Application Publication
(10) Pub. No.: US 2024/0150448 A1
(43) Pub. Date: May 9, 2024

(54) PAC1 ANTIBODIES AND USES THEREOF

(71) Applicant: AMGEN INC., Thousand Oaks, CA (US)

(72) Inventors: Neeraj Jagdish AGRAWAL, Natick, MA (US); Irwin CHEN, San Francisco, CA (US); Su CHONG, Oxnard, CA (US); Bryna FUCHSLOCHER, Thousand Oaks, CA (US); Kevin GRAHAM, Thousand Oaks, CA (US); Agnes Eva HAMBURGER, Newbury Park, CA (US); Mark Leo MICHAELS, Encino, CA (US); Christopher MOHR, Newbury Park, CA (US); Derek E. PIPER, Santa Clara, CA (US); Kenneth William WALKER, Newbury Park, CA (US); Zhulun WANG, Los Altos, CA (US); Cen XU, Newbury Park, CA (US)

(73) Assignee: AMGEN INC., Thousand Oaks, CA (US)

(21) Appl. No.: 18/545,688

(22) Filed: Dec. 19, 2023

Related U.S. Application Data
(63) Continuation of application No. 17/558,032, filed on Dec. 21, 2021, now Pat. No. 11,891,435.
Application No. 17/558,032 is a continuation of application No. 16/919,032, filed on Jul. 1, 2020, now Pat. No. 11,248,043.
Application No. 16/919,032 is a division of application No. 16/246,326, filed on Jan. 11, 2019, now Pat. No. 10,738,110.
(60) Provisional application No. 62/617,157, filed on Jan. 12, 2018.

(51) Int. Cl. C07K 16/18; A61K 38/22; A61P 25/06; C07K 16/28

(52) U.S. Cl. CPC C07K 16/18; A61K 38/2278; A61P 25/06; C07K 16/2869; A61K 2039/545; C07K 2317/34; C07K 2317/565; C07K 2317/92

(57) ABSTRACT

The present invention relates to neutralizing antibodies of the human pituitary adenylate cyclase activating polypeptide type I receptor (PAC1) and pharmaceutical compositions comprising such antibodies. Methods of treating or preventing headache conditions, such as migraine and cluster headache, using the neutralizing antibodies are also described.

### CROSS-REFERENCE TO RELATED APPLICATIONS

This application is a continuation of U.S. application Ser. No. 17/558,032, filed Dec. 21, 2021, which is a continuation of U.S. application Ser. No. 16/919,032, now U.S. Pat. No. 11,248,043, filed Jul. 1, 2020, which is a divisional of U.S. application Ser. No. 16/246,326, now U.S. Pat. No. 10,738,110, filed Jan. 11, 2019, which claims the benefit of U.S. Provisional Application No. 62/617,157, filed Jan. 12, 2018, all of which are hereby incorporated by reference in their entireties.

### DESCRIPTION OF THE TEXT FILE SUBMITTED ELECTRONICALLY

The present application contains a Sequence Listing, which has been submitted electronically in XML format and is hereby incorporated by reference in its entirety. The computer readable format copy of the Sequence Listing, which was created on Dec. 18, 2023, is named A-2189-US05-CNT_ST26 and is 815 kilobytes in size.

### FIELD OF THE INVENTION

The present invention relates to the field of biopharmaceuticals. In particular, the invention relates to antibodies that specifically bind to human pituitary adenylate cyclase-activating polypeptide type I receptor (PAC1) and potently inhibit its biological activity. The invention also relates to pharmaceutical compositions comprising the antibodies as well as methods of producing and using such antibodies.

### BACKGROUND OF THE INVENTION

Migraines are episodic headaches that can involve significant pain, are often accompanied by nausea, vomiting, and extreme sensitivity to light (photophobia) and sound (phonophobia), and are sometimes preceded by sensory warning symptoms or signs (auras). Migraine is a highly prevalent disease worldwide with approximately 12% of the European population, and 18% of women, 6% of men in the United States suffering from migraine attacks (Lipton et al, Neurology, Vol. 68:343-349, 2007; Lipton et al., Headache, Vol. 41:646-657, 2001). A study to assess the prevalence of migraine in the United States reported that nearly half the migraine patient population had three or more migraines per month (Lipton et al, Neurology, Vol. 68:343-349, 2007). Additionally, migraines are associated with a number of psychiatric and medical comorbidities such as depression and vascular disorders (Buse et al., J. Neurol. Neurosurg. Psychiatry, Vol. 81:428-432, 2010; Bigal et al., Neurology, Vol. 72:1864-1871, 2009). Most of the current migraine therapies are either not well tolerated or ineffective (Loder et al., Headache, Vol. 52:930-945, 2012; Lipton et al, 2001); thus, migraine remains an unmet medical need.

A major component of migraine pathogenesis involves the activation of the trigeminovascular system. The release of trigeminal and parasympathetic neurotransmitters from perivascular nerve fibers (Sánchez-del-Rio and Reuter, Curr. Opin. Neurol., Vol. 17(3):289-93, 2004) results in vasodilation of the cranial blood vessels and has been suggested to be associated with the onset of migraine headaches (Edvinsson, Cephalagia, Vol. 33(13): 1070-1072, 2013; Goadsby et al., New Engl J Med., Vol. 346(4):257-270, 2002).

Pituitary adenylate cyclase-activating polypeptides (PACAP) are 38-amino acid (PACAP38), or 27-amino acid (PACAP27) peptides that were first isolated from an ovine hypothalamic extract on the basis of their ability to stimulate cyclic AMP (cAMP) formation in anterior pituitary cells (Miyata et al., Biochem Biophys Res Commun., Vol. 164:567-574, 1989; Miyata et al., Biochem Biophys Res Commun., Vol. 170:643-648, 1990). PACAP belongs to the VIP/secretin/glucagon superfamily. The sequence of PACAP27 corresponds to the 27 N-terminal amino acids of PACAP38 and shares 68% identity with vasoactive intestinal polypeptide (VIP) (Pantaloni et al., J. Biol. Chem., Vol. 271: 22146-22151, 1996; Pisegna and Wank, Proc. Natl. Acad. Sci. USA, Vol. 90: 6345-49, 1993; Campbell and Scanes, Growth Regul., Vol. 2:175-191, 1992). The major form of PACAP peptide in the human body is PACAP38, and the pharmacology of PACAP38 has not been shown to be different from the pharmacology of PACAP27. Three PACAP receptors have been reported: one receptor that binds PACAP with high affinity and has a much lower affinity for VIP (PAC1 receptor), and two receptors that recognize PACAP and VIP equally well (VPAC1 and VPAC2 receptors) (Vaudry et al., Pharmacol Rev., Vol. 61:283-357, 2009).

Human experimental migraine models using PACAP as a challenge agent to induce migraine-like headaches support the approach for antagonism of the PACAP/PAC1 signaling pathway as a treatment for migraine prophylaxis. PACAP38 is elevated in plasma during spontaneous migraine attacks in migraine patients, and these elevated PACAP38 levels can be normalized with sumatriptan, an acute migraine therapy (Tuka et al., Cephalalgia, Vol. 33: 1085-1095, 2013; Zagami et al., Ann. Clin. Transl. Neurol., Vol. 1: 1036-1040, 2014). Infusion of PACAP38 causes headaches in healthy subjects and migraine-like headaches in migraine patients (Schytz et al., Brain, Vol. 132:16-25, 2009; Amin et al., Brain, Vol. 137: 779-794, 2014; Guo et al., Cephalalgia, Vol. 37:125-135, 2017). However, in the same model, VIP does not cause migraine-like headaches in migraine patients (Rahmann et al., Cephalalgia, Vol. 28:226-236, 2008). The lack of migraine-like headache induction from VIP infusion suggests that PACAP38 peptide's effects are mediated through the PAC1 receptor, rather than VPAC1 or VPAC2 receptors, because VIP has a much higher affinity at the latter two receptors. This notion is further supported by animal studies in which PAC1 receptor antagonists inhibit nociceptive neuronal activity in the trigeminocervical complex in an in vivo model of migraine (Akerman et al., Sci. Transl. Med., Vol. 7: 308ra157, 2015; Hoffmann et al., Cephalalgia, Vol. 37 (1S): 3, Abstract OC-BA-004, 2017). Taken together, these data suggest that pharmacological agents that inhibit PACAP-activation of the PAC1 receptor have the potential to treat migraine.

### SUMMARY OF THE INVENTION

The present invention is based, in part, on the design and generation of high affinity antibodies that specifically bind to and potently inhibit human PAC1. The antibodies of the invention have enhanced inhibitory potency against human PAC1 as compared to previously described anti-PAC1 antibodies, with IC50 values in the picomolar range. The isolated antibodies and antigen-binding fragments thereof can be used to inhibit, interfere with, or modulate the biological activity of human PAC1, including inhibiting or reducing PACAP-induced activation of PAC1, inhibiting or reducing vasodilation, and ameliorating or treating symptoms of migraine and other vascular headaches.

In some embodiments, the isolated antibodies and antigen-binding fragments thereof specifically bind to human PAC1 at an epitope that comprises one or more amino acids selected from Asp59, Asn60, Ile61, Arg116, Asn117, Thr119, Glu120, Asp121, Gly122, Trp123, Ser124, Glu125, Pro126, Phe127, Pro128, His129, Tyr130, Phe131, Asp132, and Gly135 of SEQ ID NO: 1. In certain embodiments, the epitope comprises at least amino acids Asn60, Ile61, Glu120, and Asp121 of human PAC1. In these and other embodiments, the antibodies and antigen-binding fragments of the invention comprise specific amino acids at particular positions within the light chain and heavy chain variable regions that interact with these epitope residues. For instance, in one embodiment, the antibodies or antigen-binding fragments comprise a light chain variable region in which the amino acid at position 29 according to AHo numbering is a basic amino acid (e.g. arginine or lysine) that interacts with amino acids Glu120 or Asp121 of human PAC1. In another embodiment, the antibodies or antigen-binding fragments comprise a heavy chain variable region in which the amino acid at position 61 according to AHo numbering is a hydrophobic, basic, or neutral hydrophilic amino acid (e.g. isoleucine, valine, leucine, glutamine, asparagine, arginine, or lysine) that interacts with amino acids Asn60 or Ile61 of human PAC1. In yet another embodiment, the antibodies or antigen-binding fragments comprise a heavy chain variable region in which the amino acid at position 66 according to AHo numbering is a basic or neutral hydrophilic amino acid (e.g. glutamine, asparagine, arginine, or lysine) that interacts with amino acids Asn60 or Ile61 of human PAC1.

The anti-PAC1 antibodies and antigen-binding fragments of the invention can inhibit ligand-induced activation of the PAC1 receptor. For instance, in some embodiments, the anti-PAC1 antibodies and antigen-binding fragments inhibit PACAP-induced activation of human PAC1 with an IC50 less than 500 pM as measured by a cell-based cAMP assay. In other embodiments, the anti-PAC1 antibodies and antigen-binding fragments inhibit PACAP-induced activation of human PAC1 with an IC50 less than 300 pM as measured by a cell-based cAMP assay. In certain embodiments, the anti-PAC1 antibodies and antigen-binding fragments inhibit PACAP-induced activation of the human PAC1 receptor with an IC50 between about 50 pM and about 500 pM as measured by a cell-based cAMP assay.

In some embodiments, the anti-PAC1 antibodies and antigen binding fragments of the invention cross-react with PAC1 receptors from other species. In one embodiment, the anti-PAC1 antibodies or antigen-binding fragments specifically bind to and inhibit PACAP-induced activation of the cynomolgus monkey PAC1 receptor. In such an embodiment, the anti-PAC1 antibodies or antigen-binding fragments may inhibit PACAP-induced activation of the cynomolgus monkey PAC1 receptor with an IC50 between about 0.1 nM and about 1 nM or between about 50 pM and about 500 pM as measured by a cell-based cAMP assay. In another embodiment, the anti-PAC1 antibodies or antigen-binding fragments specifically bind to and inhibit PACAP-induced activation of the rat PAC1 receptor. The anti-PAC1 antibodies or antigen-binding fragments may inhibit PACAP-induced activation of the rat PAC1 receptor with an IC50 less than 10 nM, for example with an IC50 between about 0.1 nM and about 10 nM or between about 100 pM and about 2 nM as measured by a cell-based cAMP assay.

In certain embodiments, the anti-PAC1 antibodies or antigen-binding fragments of the invention comprise a light chain variable region comprising complementarity determining regions CDRL1, CDRL2, and CDRL3 and a heavy chain variable region comprising complementarity determining regions CDRH1, CDRH2, and CDRH3. The light chain and heavy chain variable regions or CDRs may be from any of the anti-PAC1 antibodies described herein. For instance, in some embodiments, the anti-PAC1 antibodies or antigen-binding fragments comprise a CDRL1 comprising a sequence selected from SEQ ID NOs: 5-16; a CDRL2 comprising the sequence of SEQ ID NO: 26; a CDRL3 comprising a sequence selected from SEQ ID NOs: 36-38; a CDRH1 comprising a sequence selected from SEQ ID NOs: 88-96; a CDRH2 comprising a sequence selected from SEQ ID NOs: 106-166; and a CDRH3 comprising a sequence selected from SEQ ID NOs: 171-177. In other embodiments, the anti-PAC1 antibodies or antigen-binding fragments comprise a CDRL1 comprising a sequence selected from SEQ ID NOs: 17-25; a CDRL2 comprising a sequence selected from SEQ ID NOs: 27-35; a CDRL3 comprising a sequence selected from SEQ ID NOs: 39-51; a CDRH1 comprising a sequence selected from SEQ ID NOs: 97-105; a CDRH2 comprising a sequence selected from SEQ ID NOs: 167-170; and a CDRH3 comprising a sequence selected from SEQ ID NOs: 178-190.

In some embodiments, the anti-PAC1 antibodies or antigen-binding fragments of the invention comprise a light chain variable region that comprises a sequence that is at least 90% identical or at least 95% identical to a sequence selected from SEQ ID NOs: 54-66 and SEQ ID NOs: 68-87. In these and other embodiments, the anti-PAC1 antibodies or antigen-binding fragments of the invention comprise a heavy chain variable region that comprises a sequence that is at least 90% identical or at least 95% identical to a sequence selected from SEQ ID NOs: 191-312. In one embodiment, the anti-PAC1 antibodies or antigen-binding fragments comprise a light chain variable region comprising a sequence selected from SEQ ID NOs: 54-66 and a heavy chain variable region comprising a sequence selected from SEQ ID NOs: 191-295. In another embodiment, the anti-PAC1 antibodies or antigen-binding fragments comprise a light chain variable region comprising a sequence selected from SEQ ID NOs: 68-87 and a heavy chain variable region comprising a sequence selected from SEQ ID NOs: 296-312.

In any of the embodiments described herein, including the embodiments described above, the anti-PAC1 antibody or antigen-binding fragment of the invention is a monoclonal antibody or antigen-binding fragment thereof. In some embodiments, the monoclonal antibody or antigen-binding fragment thereof is a chimeric antibody or antigen-binding fragment thereof. In other embodiments, the monoclonal antibody or antigen-binding fragment thereof is a humanized antibody or antigen-binding fragment thereof. In yet other embodiments, the monoclonal antibody or antigen-binding fragment thereof is a fully human antibody or antigen-binding fragment thereof. The monoclonal antibody can be of any isotype, such as a human IgG1, IgG2, IgG3, or IgG4. In one particular embodiment, the monoclonal antibody is a human IgG1 antibody. In another particular embodiment, the monoclonal antibody is a human IgG2 antibody. The monoclonal antibody may comprise a light chain that comprises a human kappa constant region. In some embodiments, the human kappa constant region comprises the sequence of SEQ ID NO: 318 or SEQ ID NO: 319. Thus, the anti-PAC1 antibodies or antigen-binding fragments of the invention may comprise a light chain that comprises any of the light chain variable region sequences listed in Table 1A fused to a human kappa constant region comprising the sequence of SEQ ID NO: 318 or SEQ ID NO: 319. In other embodiments, the monoclonal antibody may comprise a light chain that comprises a human lambda constant region. In certain embodiments, the human lambda constant region comprises the sequence of SEQ ID NO: 315. Thus, in some embodiments, the anti-PAC1 antibodies or antigen-binding fragments of the invention may comprise a light chain that comprises any of the light chain variable region sequences listed in Table 1A fused to a human lambda constant region comprising the sequence of SEQ ID NO: 315.

In certain embodiments, the anti-PAC1 antibodies or antigen-binding fragments of the invention may contain one or more modifications that affect the glycosylation of the antibody or antigen-binding fragment. In some embodiments, the antibody or antigen-binding fragment comprises one or more mutations to reduce or eliminate glycosylation. In such embodiments, the aglycosylated antibody may comprise a mutation at amino acid position N297 (according to the EU numbering scheme), such as a N297G mutation, in its heavy chain. The aglycosylated antibody may comprise further mutations to stabilize the antibody structure. Such mutations can include pairs of cysteine substitutions, such as A287C and L306C, V259C and L306C, R292C and V302C, and V323C and I332C (amino acid positions according to the EU numbering scheme). In one embodiment, the aglycosylated antibody comprises R292C and V302C mutations (according to the EU numbering scheme) in its heavy chain. In certain embodiments, the aglycosylated anti-PAC1 antibody comprises a heavy chain constant region comprising the amino acid sequence of SEQ ID NO: 324 or SEQ ID NO: 325.

The present invention also includes isolated polynucleotides and expression vectors encoding the anti-PAC1 antibodies and antigen-binding fragments described herein as well as host cells, such as CHO cells, comprising the encoding polynucleotides and expression vectors. In certain embodiments, the present invention includes methods for producing the anti-PAC1 antibodies and antigen-binding fragments described herein. In one embodiment, the method comprises culturing a host cell comprising an expression vector encoding the anti-PAC1 antibody or antigen-binding fragment under conditions that allow expression of the antibody or antigen-binding fragment, and recovering the antibody or antigen-binding fragment from the culture medium or host cell.

The anti-PAC1 antibodies or antigen-binding fragments described herein can be used in the manufacture of a pharmaceutical composition or medicament for the treatment or prevention of conditions associated with PAC1 biological activity, such as headache, migraine, cluster headache, and vasomotor symptoms. Thus, the present invention also provides a pharmaceutical composition comprising an anti-PAC1 antibody or antigen-binding fragment described herein and a pharmaceutically acceptable excipient. The pharmaceutical compositions can be used in any of the methods described herein.

In certain embodiments, the present invention provides methods for treating or preventing a headache condition in a patient in need thereof comprising administering to the patient an effective amount of an anti-PAC1 antibody or antigen-binding fragment described herein. In some embodiments, the headache condition to be treated or prevented with the methods of the invention is migraine. The migraine can be episodic migraine or chronic migraine. In other embodiments, the headache condition to be treated or prevented with the methods of the invention is cluster headache. In certain embodiments, the methods provide prophylactic treatment for these conditions.

In some embodiments of the methods of the invention, the methods comprise administering a second headache therapeutic agent to the patient in combination with an anti-PAC1 antibody or antigen-binding fragment of the invention. The second headache therapeutic agent can be an acute headache therapeutic agent, such as a serotonin receptor agonist (e.g. agonist of a 5HT_{1B}, 5HT_{1D}, and/or 5HT_{1F} serotonin receptor). In some embodiments, the acute headache therapeutic agent is a triptan, ergotamine, non-steroidal anti-inflammatory drug, or an opioid. In other embodiments, the second headache therapeutic agent to be administered in combination with an anti-PAC1 antibody or antigen-binding fragment of the invention is a prophylactic headache therapeutic agent, such as an antiepileptic, a beta-blocker, an anti-depressant, or onabotulinum toxin A. The second headache therapeutic agent may be administered to the patient before, after, or concurrently with an anti-PAC1 antibody or antigen-binding fragment of the invention.

In certain embodiments of the methods of the invention, the methods comprise administering a CGRP pathway antagonist to the patient in combination with an anti-PAC1 antibody or antigen-binding fragment of the invention. The CGRP pathway antagonist can be an antagonist of the human CGRP receptor, such as an antibody that specifically binds to the human CGRP receptor. In one embodiment, the CGRP pathway antagonist administered in combination with an anti-PAC1 antibody or antigen-binding fragment of the invention is erenumab. In other embodiments, the CGRP pathway antagonist can be an antagonist of the CGRP ligand, such as an antibody that specifically binds to human α-CGRP and/or I3-CGRP. In one such embodiment, the CGRP pathway antagonist administered in combination with an anti-PAC1 antibody or antigen-binding fragment of the invention is fremanezumab. In another such embodiment, the CGRP pathway antagonist administered in combination with an anti-PAC1 antibody or antigen-binding fragment of the invention is galcanezumab. In yet another such embodiment, the CGRP pathway antagonist administered in combination with an anti-PAC1 antibody or antigen-binding fragment of the invention is eptinezumab.

The present invention also includes methods of inhibiting vasodilation in a patient in need thereof. In one embodiment, the method comprises administering to the patient an effective amount of any of the anti-PAC1 antibodies or antigen-binding fragments described herein. In some embodiments, the patient in need of treatment has a headache condition, such as migraine or cluster headache. In other embodiments, the patient in need of treatment has vasomotor symptoms (e.g. hot flashes, facial flushing, sweating, and night sweats), such as those associated with menopause.

The use of the anti-PAC1 antibodies or antigen-binding fragments in any of the methods disclosed herein or for preparation of medicaments for administration according to any of the methods disclosed herein is specifically contemplated. For instance, the present invention includes an anti-PAC1 antibody or antigen-binding fragment for use in a method for treating or preventing a headache condition in a patient in need thereof. The headache condition includes migraine (e.g. episodic and chronic migraine) and cluster headache. In some embodiments, the present invention provides an anti-PAC1 antibody or antigen-binding fragment for use in a method for inhibiting vasodilation in a patient in need thereof. In such embodiments, the patient may be diagnosed with or have a headache condition. In other embodiments, the present invention provides an anti-PAC1 antibody or antigen-binding fragment for use in a method for inhibiting activation of human PAC1 receptor in a patient having a headache condition. The headache condition may be migraine (e.g. episodic or chronic migraine) or cluster headache.

The present invention also includes the use of an anti-PAC1 antibody or antigen-binding fragment in the preparation of a medicament for treating or preventing a headache condition in a patient in need thereof. The headache condition includes migraine (e.g. episodic and chronic migraine) and cluster headache. In certain embodiments, the present invention encompasses the use of an anti-PAC1 antibody or antigen-binding fragment in the preparation of a medicament for inhibiting vasodilation in a patient in need thereof. In such embodiments, the patient may be diagnosed with or have a headache condition. In other embodiments, the present invention includes the use of an anti-PAC1 antibody or antigen-binding fragment in the preparation of a medicament for inhibiting activation of human PAC1 receptor in a patient having a headache condition. The headache condition may be migraine (e.g. episodic or chronic migraine) or cluster headache.

### BRIEF DESCRIPTION OF THE DRAWINGS

FIG. 1A is a front view of the crystal structure of the complex between human PAC1 extracellular domain (ECD) and the Fab fragment of the 29G4v9 antibody. VL=light chain variable region; CL=light chain constant region; VH=heavy chain variable region; and CH1=heavy chain CH1 constant region.

FIG. 1B is a side view of the crystal structure of the complex between human PAC1 ECD and the Fab fragment of the 29G4v9 antibody. The view depicts the structure shown in FIG. 1A rotated 90° to the left. In this view, the light chain variable region (VL) is positioned behind the heavy chain variable region (VH), and the light chain constant region (CL) is positioned behind the heavy chain CH1 constant region.

FIG. 2A is an expanded view of the interface between the human PAC1 ECD and the light chain CDR1 (amino acids 24 to 34 of SEQ ID NO: 3) of the 29G4v9 Fab. Zone 1 contains Glu120 and Asp121 residues of human PAC1 ECD (SEQ ID NO: 1), whereas Zone 3 contains the Phe127 residue of human PAC1 ECD.

FIG. 2B is an expanded view of the interface between the human PAC1 ECD and Arg93 residue in the light chain CDR3 of the 29G4v9 Fab.

FIG. 3A is an expanded view of the interface between the human PAC1 ECD and heavy chain CDR1 amino acids Arg31 and Phe32 of the 29G4v9 Fab. The two heavy chain CDR1 amino acids lie on either side of the Phe131 residue of human PAC1 ECD.

FIG. 3B is an expanded view of the interface between a region of the human PAC1 ECD containing amino acid residues Asn60 and Ile61 (relative to SEQ ID NO: 1) and heavy chain CDR2 amino acids Tyr53, Asp54, and Gly56 of the 29G4v9 Fab.

FIG. 3C is an expanded view of the interface between the human PAC1 ECD and heavy chain CDR3 amino acids Val102, Leu103, and Thr104 of the 29G4v9 Fab.

FIG. 4 is a schematic of the selection process for improved binding mutants from a yeast-displayed antibody Fab mutant library.

FIG. 5 is a schematic of the binding off-rate driven selection process for high affinity binding mutants from a yeast-displayed library.

FIG. 6 shows the inhibitory effect of anti-PAC1 antibodies on maxadilan-induced increase in dermal blood flow in rats. One of seven antibodies (420653, 420845, 420943, 421873, 420889 (PL-50347), 421091 (PL-50350), and 421051 (PL-50351)) was administered to rats intravenously at a dose of 0.1 mg/kg or 0.3 mg/kg twenty-four hours prior to challenge with 10 ng maxadilan (intradermal injection). Dermal blood flow was assessed 30 minutes following maxadilan challenge with laser Doppler imaging. *p<0.05, **p<0.01 compared to the vehicle group with One-Way ANOVA followed by Dunnett's MCT.

FIG. 7A shows the dose-dependent effect of anti-PAC1 antibody 420653 on maxadilan-induced increase in dermal blood flow in rats. The antibody was administered to rats intravenously at one of seven doses ranging from 0.01 mg/kg to 3 mg/kg twenty-four hours prior to challenge with 10 ng maxadilan (intradermal injection). Dermal blood flow was assessed 30 minutes following maxadilan challenge with laser Doppler imaging. *p<0.05, ****p<0.0001 compared to the vehicle group with One-Way ANOVA followed by Dunnett's MCT.

FIG. 7B shows the dose-dependent effect of anti-PAC1 antibody 420845 on maxadilan-induced increase in dermal blood flow in rats. The antibody was administered to rats intravenously at one of five doses ranging from 0.01 mg/kg to 3 mg/kg twenty-four hours prior to challenge with 10 ng maxadilan (intradermal injection). Dermal blood flow was assessed 30 minutes following maxadilan challenge with laser Doppler imaging. ****p<0.0001 compared to the vehicle group with One-Way ANOVA followed by Dunnett's MCT.

FIG. 7C shows the dose-dependent effect of anti-PAC1 antibody 420943 on maxadilan-induced increase in dermal blood flow in rats. The antibody was administered to rats intravenously at one of six doses ranging from 0.1 mg/kg to 30 mg/kg twenty-four hours prior to challenge with 10 ng maxadilan (intradermal injection). Dermal blood flow was assessed 30 minutes following maxadilan challenge with laser Doppler imaging. ****p<0.0001 compared to the vehicle group with One-Way ANOVA followed by Dunnett's MCT.

FIG. 7D shows the dose-dependent effect of anti-PAC1 antibody 421873 on maxadilan-induced increase in dermal blood flow in rats. The antibody was administered to rats intravenously at one of five doses ranging from 0.3 mg/kg to 30 mg/kg twenty-four hours prior to challenge with 10 ng maxadilan (intradermal injection). Dermal blood flow was assessed 30 minutes following maxadilan challenge with laser Doppler imaging. *p<0.05, ****p<0.0001 compared to the vehicle group with One-Way ANOVA followed by Dunnett's MCT.

FIG. 8A is the serum concentration-time profile for anti-PAC1 antibodies 420653, 420845, 420943, and 421873 following a single intravenous dose of 1 mg/kg in rats.

FIG. 8B is the serum concentration-time profile for anti-PAC1 antibodies 420653, 420845, 420943, and 421873 following a single intravenous dose of 5 mg/kg in rats.

FIG. 8C is the serum concentration-time profile for anti-PAC1 antibodies 420653, 420845, 420943, and 421873 following a single intravenous dose of 25 mg/kg in rats.

FIG. 9 is the serum concentration-time profile for anti-PAC1 antibodies 420653, 420845, 420943, 421873, and 29G4v22 following a single intravenous dose of 10 mg/kg in cynomolgus monkeys.

FIGS. 10A and 10B show the time-course of the inhibitory effects of anti-PAC1 antibodies 420653 and 29G4v22 on maxadilan-induced increase in dermal blood flow in cynomolgus monkeys. Twenty-four cynomolgus monkeys were given a single i.v. bolus injection of antibody 420653 at 0.1 mg/kg or 3 mg/kg or antibody 29G4v22 at 10 mg/kg at Day 0. On Days 2, 4, 7, 10, 14, 21, 28, and 36 following antibody dosing, post-maxadilan responses were measured. In each case, dermal blood flow was measured by laser Doppler imaging 30 minutes following 1 ng maxadilan intradermal injection. All data are expressed as the mean±standard error of the mean. FIG. 10A shows the % change from baseline in dermal blood flow induced by intradermal injection of maxadilan at the indicated days following antibody treatment. *p<0.05, **p<0.01, ***p<0.001 compared to Day 0 within the same treatment group by one-way ANOVA followed by Bonferroni's Multiple Comparison Test. Eight animals per group except for Day 36 in which four animals were tested with antibody 420653 at 3 mg/kg as denoted by the A symbol. FIG. 10B shows the inhibitory effect of antibody treatment on maxadilan-induced increase in dermal blood flow expressed as % inhibition. #p<0.05, ##p<0.01, ###p<0.001 comparison between antibody 420653 at 3 mg/kg and antibody 29G4v22 at 10 mg/kg at each time point by two-tailed Unpaired t-test.

### DETAILED DESCRIPTION

The present invention relates to isolated antibodies and antigen-binding fragments thereof that specifically bind to pituitary adenylate cyclase-activating polypeptide type I receptor (PAC1), particularly human PAC1. The antibodies of the invention have enhanced inhibitory potency against human PAC1 as compared to previously described anti-PAC1 antagonist antibodies. The antibodies of the invention also cross-react with the rat PAC1 receptor and the cynomolgus monkey PAC1 receptor, thereby allowing for preclinical in vivo evaluation of the antibodies in these species.

Human PAC1 is a 468 amino acid protein (NCBI Reference Sequence NP 001109.2) encoded by the ADCYAP IRI gene on chromosome 7. The human PAC1 receptor is a G protein-coupled receptor that is positively coupled to adenylate cyclase. Activation of the human PAC1 receptor by its endogenous ligands (e.g. PACAP38 or PACAP27) results in an increase in intracellular cyclic AMP (cAMP). The amino acid sequence for human PAC1 is provided below as SEQ ID NO: 1.

| | |
|---|---|
| 1 | MAGVVHVSLA ALLLLPMAPA MHSDCIFKKE QAMCLEKIQR ANELMGFNDS SPGCPGMWDN |
| 61 | ITCWKPAHVG EMVLVSCPEL FRIFNPDQVW ETETIGESDF GDSNSLDLSD MGVVSRNCTE |
| 121 | DGWSEPFPHY FDACGFDEYE SETGDQDYYY LSVKALYTVG YSTSLVTLTT AMVILCRFRK |
| 181 | LHCTRNFIHM NLFVSFMLRA ISVFIKDWIL YAEQDSNHCF ISTVECKAVM VFFHYCVVSN |
| 241 | YFWLFIEGLY LFTLLVETFF PERRYFYWYT IIGWGTPTVC VTVWATLRLY FDDTGCWDMN |
| 301 | DSTALWWVIK GPVVGSIMVN FVLFIGIIVI LVQKLQSPDM GGNESSIYLR LARSTLLLIP |
| 361 | LFGIHYTVFA FSPENVSKRE RLVFELGLGS FQGFVVAVLY CFLNGEVQAE IKRKWRSWKV |
| 421 | NRYFAVDFKH RHPSLASSGV NGGTQLSILS KSSSQIRMSG LPADNLAT |

Amino acids 1 to 23 of the human PAC1 protein (SEQ ID NO: 1) constitute a signal peptide, which is generally removed from the mature protein. The mature human PAC1 protein has the basic structure of a G protein-coupled receptor consisting of a seven-transmembrane domain, an extracellular domain composed of an N-terminal region and three extracellular loops, three intracellular loops, and a C-terminal cytoplasmic domain. The N-terminal extracellular domain is approximately at amino acids 24-153 of SEQ ID NO: 1, and the first of seven transmembrane domains begins at amino acid 154 of SEQ ID NO: 1. The C-terminal cytoplasmic domain is located approximately at amino acids 397-468 of SEQ ID NO: 1. See Blechman and Levkowitz, Front. Endocrinol., Vol. 4 (55): 1-19, 2013 for location of domains within the amino acid sequence. The terms “human PAC1,” “human PAC1 receptor,” “hPAC1,” and “hPAC1 receptor” are used interchangeably and can refer to a polypeptide of SEQ ID NO: 1, a polypeptide of SEQ ID NO: 1 minus the signal peptide (amino acids 1 to 23), allelic variants of human PAC1, or splice variants of human PAC1.

The present invention provides antibodies that specifically bind to human PAC1. An “antibody” is a protein that comprises an antigen-binding fragment that specifically binds to an antigen, and a scaffold or framework portion that allows the antigen-binding fragment to adopt a conformation that promotes binding of the antibody to the antigen. As used herein, the term “antibody” generally refers to a tetrameric immunoglobulin protein comprising two light chain polypeptides (about 25 kDa each) and two heavy chain polypeptides (about 50-70 kDa each).

The term “light chain” or “immunoglobulin light chain” refers to a polypeptide comprising, from amino terminus to carboxyl terminus, a single immunoglobulin light chain variable region (VL) and a single immunoglobulin light chain constant domain (CL). The immunoglobulin light chain constant domain (CL) can be a human kappa (κ) or human lambda (λ) constant domain. The term “heavy chain” or “immunoglobulin heavy chain” refers to a polypeptide comprising, from amino terminus to carboxyl terminus, a single immunoglobulin heavy chain variable region (VH), an immunoglobulin heavy chain constant domain 1 (CH1), an immunoglobulin hinge region, an immunoglobulin heavy chain constant domain 2 (CH2), an immunoglobulin heavy chain constant domain 3 (CH3), and optionally an immunoglobulin heavy chain constant domain 4 (CH4). Heavy chains are classified as mu (p), delta (A), gamma (γ), alpha (a), and epsilon (c), and define the antibody's isotype as IgM, IgD, IgG, IgA, and IgE, respectively. The IgG-class and IgA-class antibodies are further divided into subclasses, namely, IgG1, IgG2, IgG3, and IgG4, and IgA1 and IgA2, respectively. The heavy chains in IgG, IgA, and IgD antibodies have three constant domains (CH1, CH2, and CH3), whereas the heavy chains in IgM and IgE antibodies have four constant domains (CH1, CH2, CH3, and CH4). The immunoglobulin heavy chain constant domains can be from any immunoglobulin isotype, including subtypes. The antibody chains are linked together via inter-polypeptide disulfide bonds between the CL domain and the CH1 domain (i.e. between the light and heavy chain) and between the hinge regions of the two antibody heavy chains.

The present invention also includes antigen-binding fragments of the anti-PAC1 antibodies described herein. An “antigen-binding fragment,” used interchangeably herein with “binding fragment” or “fragment,” is a portion of an antibody that lacks at least some of the amino acids present in a full-length heavy chain and/or light chain, but which is still capable of specifically binding to an antigen. An antigen-binding fragment includes, but is not limited to, a single-chain variable fragment (scFv), a nanobody (e.g. VH domain of camelid heavy chain antibodies; VHH fragment, see Cortez-Retamozo et al., Cancer Research, Vol. 640:2853-57, 2004), a Fab fragment, a Fab′ fragment, a F(ab′)2 fragment, a Fv fragment, a Fd fragment, and a complementarity determining region (CDR) fragment, and can be derived from any mammalian source, such as human, mouse, rat, rabbit, or camelid. Antigen-binding fragments may compete for binding of a target antigen with an intact antibody and the fragments may be produced by the modification of intact antibodies (e.g. enzymatic or chemical cleavage) or synthesized de novo using recombinant DNA technologies or peptide synthesis. In some embodiments, the antigen-binding fragment comprises at least one CDR from an antibody that binds to the antigen, for example, the heavy chain CDR3 from an antibody that binds to the antigen. In other embodiments, the antigen-binding fragment comprises all three CDRs from the heavy chain of an antibody that binds to the antigen or all three CDRs from the light chain of an antibody that binds to the antigen. In still other embodiments, the antigen-binding fragment comprises all six CDRs from an antibody that binds to the antigen (three from the heavy chain and three from the light chain).

The term “isolated molecule” (where the molecule is, for example, a polypeptide, a polynucleotide, an antibody, or antigen-binding fragment) is a molecule that by virtue of its origin or source of derivation (1) is not associated with naturally associated components that accompany it in its native state, (2) is substantially free of other molecules from the same species (3) is expressed by a cell from a different species, or (4) does not occur in nature. Thus, a molecule that is chemically synthesized, or expressed in a cellular system different from the cell from which it naturally originates, will be “isolated” from its naturally associated components. A molecule also may be rendered substantially free of naturally associated components by isolation, using purification techniques well known in the art. Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

In certain embodiments of the invention, the antibodies or antigen-binding fragments thereof specifically bind to human PAC1. An antibody or antigen-binding fragment thereof “specifically binds” to a target antigen when it has a significantly higher binding affinity for, and consequently is capable of distinguishing, that antigen compared to its affinity for other unrelated proteins, under similar binding assay conditions. Antibodies or antigen-binding fragments that specifically bind an antigen may have an equilibrium dissociation constant (K_{D})≤1×10⁻⁶ M. The antibody or binding fragment specifically binds antigen with “high affinity” when the K_{D} is ≤1×10⁻⁸ M. In one embodiment, the antibodies or binding fragments of the invention bind to human PAC1 with a K_{D} of ≤5×10⁻⁹ M. In another embodiment, the antibodies or binding fragments of the invention bind to human PAC1 with a K_{D} of ≤1×10⁻⁹ M. In yet another embodiment, the antibodies or binding fragments of the invention bind to human PAC1 with a K_{D} of ≤5×10 40 M. In another embodiment, the antibodies or binding fragments of the invention bind to human PAC1 with a K_{D} of ≤1×10⁻¹⁰ M. In certain embodiments, the antibodies or binding fragments of the invention bind to human PAC1 with a K_{D} of ≤5×10⁻¹¹ M. In other embodiments, the antibodies or binding fragments of the invention bind to human PAC1 with a K_{D} of ≤1×10⁻¹¹ M. In one particular embodiment, the antibodies or binding fragments of the invention bind to human PAC1 with a K_{D} of ≤5×10⁻¹² M. In another particular embodiment, the antibodies or binding fragments of the invention bind to human PAC1 with a K_{D} of ≤1×10⁻¹² M.

Affinity is determined using a variety of techniques, an example of which is an affinity ELISA assay. In various embodiments, affinity is determined by a surface plasmon resonance assay (e.g., BIAcore®-based assay). Using this methodology, the association rate constant (kₐ in M⁻¹ s⁻¹) and the dissociation rate constant (k_{d} ins 1) can be measured. The equilibrium dissociation constant (K_{D} in M) can then be calculated from the ratio of the kinetic rate constants (k_{d}/kₐ). In some embodiments, affinity is determined by a kinetic method, such as a Kinetic Exclusion Assay (KinExA) as described in Rathanaswami et al. Analytical Biochemistry, Vol. 373:52-60, 2008. Using a KinExA assay, the equilibrium dissociation constant (K_{D} in M) and the association rate constant (kₐ in M⁻¹ s⁻¹) can be measured. The dissociation rate constant (k_{d} in s⁻¹) can be calculated from these values (K_{D}×kₐ). In other embodiments, affinity is determined by a bio-layer interferometry method, such as that described in Kumaraswamy et al., Methods Mol. Biol., Vol. 1278:165-82, 2015 and employed in Octet® systems (Pall ForteBio). The kinetic (kₐ and k_{d}) and affinity (K_{D}) constants can be calculated in real-time using the bio-layer interferometry method. In some embodiments, the antibodies or binding fragments described herein exhibit desirable characteristics such as binding avidity as measured by k_{d} (dissociation rate constant) for human PAC1 of about 10⁻², 10⁻³, 10⁻⁴, 10⁻⁵, 10⁻⁶, 10⁻⁷, 10⁻⁸, 10⁻⁹, 10⁻¹⁰ s⁻¹ or lower (lower values indicating higher binding avidity), and/or binding affinity as measured by K_{D} (equilibrium dissociation constant) for human PAC1 of about 10⁻⁸, 10⁻⁹, 10⁻¹⁰, 10⁻¹¹, 10⁻¹² or lower (lower values indicating higher binding affinity).

Preferably, the antibodies or binding fragments of the invention do not significantly bind to or cross-react with other members of the secretin/glucagon receptor family, such as human VPAC1 receptor (NCBI Reference Sequence NP_004615.2) and human VPAC2 receptor (NCBI Reference Sequence NP_003373.2). As used herein, an antibody or binding fragment does “not significantly bind” to a target antigen when it has a binding affinity for that antigen that is comparable to its affinity for other unrelated proteins, under similar binding assay conditions. Antibodies or binding fragments that do not significantly bind to a target antigen may also include those proteins that do not generate a statistically different signal than a negative control in an affinity assay, such as those described herein, for the target antigen. By way of example, an antibody, which produces a signal value in an ELISA- or a BIAcore®-based assay for determining binding to human VPAC1 that is not statistically different from the signal value produced with a negative control (e.g. buffer solution without antibody), would be considered to not significantly bind to human VPAC1. Antibodies or binding fragments that do not significantly bind an antigen may have an equilibrium dissociation constant (K_{D}) for that antigen greater than 1×10⁻⁶ M, greater than 1×10⁻⁵ M, greater than 1×10⁻⁴ M, or greater than 1×10⁻³ M. Thus, in certain embodiments, the antibodies and binding fragments of the invention selectively bind to human PAC1 relative to human VPAC1 and human VPAC2. In other words, the antibodies and binding fragments of the invention do not significantly bind to human VPAC1 or human VPAC2.

The antibodies and antigen-binding fragments of the invention may inhibit, interfere with, or modulate one or more biological activities of the human PAC1 receptor. Biological activities of the human PAC1 receptor include, but are not limited to, induction of PACAP-mediated receptor signal transduction pathways, induction of vasodilation, and inhibition of vasoconstriction. In some embodiments, the antibodies or binding fragments of the invention inhibit binding of PACAP (e.g. PACAP38 or PACAP27) to the human PAC1 receptor. “Inhibition of binding” occurs when an excess of antibodies or binding fragments reduces the quantity of human PAC1 receptor bound to PACAP, or vice versa, for example, by at least about 40%, about 50%, about 60%, about 70%, about 80%, about 85%, about 90%, about 95%, about 97%, about 99% or more, for example by measuring binding in an in vitro competitive binding assay. Inhibitory constants (Ki), which are indicative of how potent the antibodies or antigen-binding fragments of the invention are at preventing binding of PACAP to human PAC1 receptor, can be calculated from such competitive binding assays. By way of example, a radioactive isotope (e.g. 125I) is attached to the receptor ligand (e.g. PACAP38) and the assay measures the binding of the radiolabeled ligand to human PAC1 receptor in increasing concentrations of the anti-PAC1 antibody or binding fragment thereof. The Ki value can be calculated using the equation Ki=IC50/(1+([L]/Kd)), where [L] is the concentration of the radioligand used (e.g., ¹²⁵I-labeled PACAP38) and Kd is the dissociation constant of the radioligand. See, e.g., Keen M, MacDermot J (1993) Analysis of receptors by radioligand binding. In: Wharton J, Polak J M (eds) Receptor autoradiography, principles and practice. Oxford University Press, Oxford. The lower the value of Ki for an antagonist, the more potent the antagonist is. In some embodiments, the antibodies or antigen-binding fragments thereof of the invention compete for binding to the human PAC1 receptor with a radiolabeled PACAP ligand with a Ki of ≤1 nM. In other embodiments, the antibodies or antigen-binding fragments thereof of the invention compete for binding to the human PAC1 receptor with a radiolabeled PACAP ligand with a Ki of ≤500 pM. In yet other embodiments, the antibodies or antigen-binding fragments thereof of the invention compete for binding to the human PAC1 receptor with a radiolabeled PACAP ligand with a Ki of ≤200 pM. In certain other embodiments, the antibodies or antigen-binding fragments thereof of the invention compete for binding to the human PAC1 receptor with a radiolabeled PACAP ligand with a Ki of ≤100 pM.

In certain embodiments, the antibodies and antigen-binding fragments of the invention inhibit ligand-induced activation of the human PAC1 receptor. The ligand can be an endogenous ligand of the receptor, such as PACAP38 or PACAP27, or the ligand can be another known agonist of the receptor, such as maxadilan. Maxadilan is a 65 amino acid peptide originally isolated from the sand fly that is exquisitely selective for PAC1 compared with VPAC1 or VPAC2, and can thus be used as a PAC1-selective agonist (Lerner et al., J Biol Chem., Vol. 266(17):11234-11236, 1991; Lerner et al., Peptides, Vol. 28(9): 1651-1654, 2007). Various assays for assessing activation of PAC1 receptors are known in the art and include cell-based assays measuring ligand-induced calcium mobilization and cAMP production. An exemplary cell-based cAMP assay is described in Example 3. Other suitable PAC1 receptor activation assays are described in Dickson et al., Ann. N. Y. Acad. Sci., Vol. 1070:239-42, 2006; Bourgault et al., J. Med. Chem., Vol. 52: 3308-3316, 2009; and U.S. Patent Publication No. 2011/0229423, all of which are hereby incorporated by reference in their entireties.

The inhibitory activity of the antibodies or antigen-binding fragments on PAC1 receptor activation can be quantitated by calculating an IC50 in any functional assay for the receptor, such as those described above. An “IC50” is the dose/concentration required to achieve 50% inhibition of a biological or biochemical function. With radioactive ligands, IC50 is the concentration of a competing ligand that displaces 50% of the specific binding of the radioligand. The IC50 of any particular substance or antagonist can be determined by constructing a dose-response curve and examining the effect of different concentrations of the drug or antagonist on reversing agonist activity in a particular functional assay. IC50 values can be calculated for a given antagonist or drug by determining the concentration needed to inhibit half of the maximum biological response of the agonist. Thus, the IC50 value for any anti-PAC1 antibody or binding fragment thereof of the invention can be calculated by determining the concentration of the antibody or binding fragment needed to inhibit half of the maximum biological response of the ligand (e.g. PACAP27, PACAP38, or maxadilan) in activating the human PAC1 receptor in any functional assay, such as the cAMP assay described in the Examples. An anti-PAC1 antibody or binding fragment thereof that inhibits ligand-induced (e.g. PACAP-induced) activation of the PAC1 receptor is understood to be a neutralizing or antagonist antibody or binding fragment of the PAC1 receptor.

In certain embodiments, the antibodies or antigen-binding fragments of the invention inhibit PACAP-induced (PACAP38- or PACAP27-induced) activation of the human PAC1 receptor. For instance, the antibodies or antigen-binding fragments may inhibit PACAP-induced activation of the human PAC1 receptor with an IC50 less than about 10 nM, less than about 8 nM, less than about 5 nM, less than about 3 nM, less than about 1 nM, less than about 800 pM, less than about 500 pM, less than about 400 pM, less than about 300 pM, less than about 200 pM, or less than about 100 pM as measured by a cell-based calcium mobilization assay or cAMP assay. In one particular embodiment, the antibodies or antigen-binding fragments of the invention inhibit PACAP-induced activation of the human PAC1 receptor with an IC50 less than about 5 nM as measured by a cell-based cAMP assay. In another particular embodiment, the antibodies or antigen-binding fragments of the invention inhibit PACAP-induced activation of the human PAC1 receptor with an IC50 less than about 1 nM as measured by a cell-based cAMP assay. In still another particular embodiment, the antibodies or antigen-binding fragments of the invention inhibit PACAP-induced activation of the human PAC1 receptor with an IC50 less than about 500 pM as measured by a cell-based cAMP assay. In another embodiment, the antibodies or antigen-binding fragments of the invention inhibit PACAP-induced activation of the human PAC1 receptor with an IC50 less than about 300 pM as measured by a cell-based cAMP assay. In some embodiments, the antibodies or antigen-binding fragments of the invention inhibit PACAP-induced activation of the human PAC1 receptor with an IC50 between about 0.1 nM and about 1 nM as measured by a cell-based cAMP assay. In other embodiments, the antibodies or antigen-binding fragments of the invention inhibit PACAP-induced activation of the human PAC1 receptor with an IC50 between about 50 pM and about 500 pM as measured by a cell-based cAMP assay.

In some embodiments, the antibodies or antigen-binding fragments of the invention bind to and inhibit PAC1 receptors from other species. For instance, the antibodies or antigen-binding fragments bind to and inhibit the cynomolgus monkey PAC1 receptor (NCBI Reference Sequence XP 015303041.1). In such embodiments, the antibodies or antigen-binding fragments inhibit PACAP-induced activation of the cynomolgus monkey PAC1 receptor with an IC50 less than about 10 nM, less than about 8 nM, less than about 5 nM, less than about 3 nM, less than about 1 nM, less than about 800 pM, less than about 500 pM, less than about 400 pM, less than about 300 pM, less than about 200 pM, or less than about 100 pM as measured by a cell-based calcium mobilization assay or cAMP assay. In one embodiment, the antibodies or antigen-binding fragments of the invention inhibit PACAP-induced activation of the cynomolgus monkey PAC1 receptor with an IC50 less than about 1 nM as measured by a cell-based cAMP assay. In another embodiment, the antibodies or antigen-binding fragments of the invention inhibit PACAP-induced activation of the cynomolgus monkey PAC1 receptor with an IC50 less than about 500 pM as measured by a cell-based cAMP assay. In yet another embodiment, the antibodies or antigen-binding fragments of the invention inhibit PACAP-induced activation of the cynomolgus monkey PAC1 receptor with an IC50 between about 0.1 nM and about 1 nM as measured by a cell-based cAMP assay. In still another embodiment, the antibodies or antigen-binding fragments of the invention inhibit PACAP-induced activation of the cynomolgus monkey PAC1 receptor with an IC50 between about 50 pM and about 500 pM as measured by a cell-based cAMP assay.

In certain embodiments, the antibodies or antigen-binding fragments bind to and inhibit the rat PAC1 receptor (NCBI Reference Sequence NP_598195.1). In these embodiments, the antibodies or antigen-binding fragments may inhibit PACAP-induced activation of the rat PAC1 receptor with an IC50 less than about 10 nM, less than about 8 nM, less than about 5 nM, less than about 3 nM, less than about 1 nM, less than about 800 pM, less than about 500 pM, less than about 400 pM, less than about 300 pM, less than about 200 pM, or less than about 100 pM as measured by a cell-based calcium mobilization assay or cAMP assay. In one embodiment, the antibodies or antigen-binding fragments of the invention inhibit PACAP-induced activation of the rat PAC1 receptor with an IC50 less than about 10 nM as measured by a cell-based cAMP assay. In another embodiment, the antibodies or antigen-binding fragments of the invention inhibit PACAP-induced activation of the rat PAC1 receptor with an IC50 less than about 5 nM as measured by a cell-based cAMP assay. In yet another embodiment, the antibodies or antigen-binding fragments of the invention inhibit PACAP-induced activation of the rat PAC1 receptor with an IC50 less than about 500 pM as measured by a cell-based cAMP assay. In still another embodiment, the antibodies or antigen-binding fragments of the invention inhibit PACAP-induced activation of the rat PAC1 receptor with an IC50 less than about 300 pM as measured by a cell-based cAMP assay. In some embodiments, the antibodies or antigen-binding fragments of the invention inhibit PACAP-induced activation of the rat PAC1 receptor with an IC50 between about 0.1 nM and about 10 nM as measured by a cell-based cAMP assay. In still another embodiment, the antibodies or antigen-binding fragments of the invention inhibit PACAP-induced activation of the rat PAC1 receptor with an IC50 between about 100 pM and about 2 nM as measured by a cell-based cAMP assay. In some embodiments in which the antibodies or binding fragments of the invention cross-react with PAC1 receptors from other species, the antibodies or binding fragments may inhibit PACAP-induced activation of the PAC1 receptor with comparable potencies. For example, an antibody or binding fragment of the invention may inhibit PACAP-induced activation of the human PAC1 receptor with an IC50 similar to the IC50 for the antibody or binding fragment to inhibit PACAP-induced activation of the cynomolgus monkey or rat PAC1 receptor. Cross-reactivity to PAC1 receptors of other species of the antibodies or binding fragments of the invention can be advantageous as the antibodies or binding fragments can be evaluated in additional pre-clinical animal models for therapeutic efficacy.

Generally, the antibodies or antigen-binding fragments of the invention do not significantly inhibit ligand-induced activation (e.g. PACAP38-, PACAP27-, or VIP-induced) of the human VPAC1 receptor or VPAC2 receptor. As used herein, an antibody or antigen-binding fragment would “not significantly inhibit” the activation of a receptor or binding of a ligand to its receptor if there is no statistical difference between ligand-induced receptor activation or ligand binding to the receptor in the presence or absence of the antibody or antigen-binding fragment. For example, if the amount of cAMP production induced by PACAP or VIP in cells expressing human VPAC1 receptor in the presence of an antibody or binding fragment is not statistically different than the amount produced in the absence of the antibody or binding fragment, then the antibody or binding fragment would be considered to not significantly inhibit PACAP/VIP-induced activation of the human VPAC1 receptor. Similarly, if the amount of PACAP or VIP bound to the human VPAC1 receptor in the presence of excess antibody or binding fragment is not statistically different than the amount of PACAP or VIP bound to the receptor in the absence of the antibody or binding fragment, then the antibody or binding fragment would be considered to not significantly inhibit the binding of PACAP or VIP to the human VPAC1 receptor. In certain embodiments, the antibodies or binding fragments of the invention inhibit PACAP-induced activation of the human PAC1 receptor, but do not significantly inhibit PACAP-induced activation of the human VPAC1 receptor or the human VPAC2 receptor. Thus, the antibodies and binding fragments of the invention selectively inhibit the human PAC1 receptor relative to the human VPAC1 receptor and the human VPAC2 receptor.

The antibodies and binding fragments of the invention may, in some embodiments, bind to a particular region or epitope of human PAC1. As used herein, an “epitope” refers to any determinant capable of being specifically bound by an antibody or fragment thereof. An epitope is a region of an antigen that is bound by, or interacts with, an antibody or binding fragment that targets that antigen, and when the antigen is a protein, includes specific amino acids that directly contact, or interact with, the antibody or binding fragment. An epitope can be formed both by contiguous amino acids or non-contiguous amino acids juxtaposed by tertiary folding of a protein. A “linear epitope” is an epitope where an amino acid primary sequence comprises the recognized epitope. A linear epitope typically includes at least 3 or 4 amino acids, and more usually, at least 5, at least 6, or at least 7 amino acids, for example, about 8 to about 10 amino acids in a unique sequence. A “conformational epitope,” in contrast to a linear epitope, is a group of discontinuous amino acids (e.g., in a polypeptide, amino acid residues that are not contiguous in the polypeptide's primary sequence but that, in the context of the polypeptide's tertiary and quaternary structure, are near enough to each other to be bound by an antibody or binding fragment thereof). Epitope determinants can include chemically active surface groupings of molecules such as amino acids, sugar side chains, phosphoryl or sulfonyl groups, and can have specific three-dimensional structural characteristics, and/or specific charge characteristics. Generally, antibodies or binding fragments specific for a particular target molecule will preferentially recognize an epitope on the target molecule in a complex mixture of proteins and/or macromolecules.

In certain embodiments, the antibodies or antigen-binding fragments of the invention bind to human PAC1 at an epitope within the N-terminal extracellular domain (ECD) of human PAC1 (SEQ ID NO: 4). In related embodiments, the antibodies or antigen-binding fragments bind to human PAC1 at an epitope within amino acids 24-153 of SEQ ID NO: 1. In other related embodiments, the antibodies or antigen-binding fragments bind to human PAC1 at an epitope within amino acids 50-140 of SEQ ID NO: 1. As described in Example 1, a crystal structure of the complex of the human PAC1 N-terminal ECD and the Fab region of an anti-PAC1 neutralizing antibody revealed key amino acids within the human PAC1 ECD that comprised the binding interface with the anti-PAC1 Fab. These core interface amino acids, all of which contained at least one non-hydrogen atom at a distance of 5 Å or less from a non-hydrogen atom in the Fab, include Asp59, Asn60, Ile61, Arg116, Asn117, Thr119, Asp121, Gly122, Trp123, Ser124, Glu125, Pro126, Phe127, Pro128, His129, Tyr130, Phe131, Asp132, and Gly135 (amino acid position numbers relative to SEQ ID NO: 1). Thus, in some embodiments, the antibodies or binding fragments of the invention bind to human PAC1 at an epitope comprising one or more amino acids selected from Asp59, Asn60, Ile61, Arg116, Asn117, Thr119, Glu120, Asp121, Gly122, Trp123, Ser124, Glu125, Pro126, Phe127, Pro128, His129, Tyr130, Phe131, Asp132, and Gly135 of SEQ ID NO: 1. In other embodiments, the antibodies or binding fragments bind to human PAC1 at an epitope comprising at least amino acids Asn60, Ile61, Glu120, and Asp121 of SEQ ID NO: 1. In yet other embodiments, the antibodies or binding fragments bind to human PAC1 at an epitope comprising at least amino acids Asn60, Ile61, Glu120, Asp121, Phe127, and Phe131 of SEQ ID NO: 1. In certain other embodiments, the antibodies or binding fragments bind to human PAC1 at an epitope comprising all of the amino acids selected from Asp59, Asn60, Ile61, Arg116, Asn117, Thr119, Glu120, Asp121, Gly122, Trp123, Ser124, Glu125, Pro126, Phe127, Pro128, His129, Tyr130, Phe131, Asp132, and Gly of SEQ ID NO: 1.

The crystal structure of the human PAC1 ECD-Fab complex described in Example 1 also revealed important residues in the CDRs of the heavy and light chains of the Fab that interacted with the amino acids in the human PAC1 ECD, thereby identifying key amino acids in the paratope of the antibody. A “paratope” is the region of an antibody that recognizes and binds to the target antigen. Paratope residues include Gln27, Gly30, Arg31, and Ser32 in the light chain variable region (SEQ ID NO: 3) and Arg31, Phe32, Tyr53, Asp54, Gly56 in the heavy chain variable region (SEQ ID NO: 2). Specific mutations of several of these residues in the paratope were designed to improve the interaction with the core interface residues (i.e. residues in the epitope) in the human PAC1 ECD resulting in enhanced binding affinity and inhibitory potency as compared to the parental antibody (see Examples 1-3). Gln27, Gly30, Arg31, and Ser32 in the light chain variable region of SEQ ID NO: 3 or SEQ ID NO: 52 correspond to amino acids positions 29, 32, 39, and 40 in AHo numbering, respectively, and Arg31, Phe32, Tyr53, Asp54, Gly56 in the heavy chain variable region of SEQ ID NO: 2 or SEQ ID NO: 191 correspond to amino acid positions 33, 39, 60, 61, and 66 in AHo numbering, respectively. The AHo numbering scheme is a structure-based numbering scheme, which introduces gaps in the CDR regions to minimize deviation from the average structure of the aligned domains (Honegger and Pluckthun, J. Mol. Biol. 309(3):657-670; 2001). In the AHo numbering scheme, structurally equivalent positions in different antibodies will have the same residue number.

In some embodiments, the present invention provides an antibody or antigen-binding fragment thereof that specifically binds to human PAC1 (SEQ ID NO: 1), wherein the antibody or antigen-binding fragment thereof comprises: (i) a light chain variable region in which the amino acid at position 29 according to AHo numbering is a basic amino acid that interacts with amino acids Glu120 or Asp121 of human PAC1, and (ii) a heavy chain variable region in which the amino acid at position 61 according to AHo numbering is a hydrophobic, basic, or neutral hydrophilic amino acid that interacts with amino acids Asn60 or Ile61 of human PAC1. As used herein, one amino acid is said to “interact” with another amino acid when one or more atoms in one amino acid forms non-covalent bonds with one or more atoms in the other amino acid through, for example, van der Waals, hydrophobic, or electrostatic forces. Basic amino acids include arginine, lysine, and histidine, whereas neutral hydrophilic amino acids include asparagine, glutamine, serine, and threonine. Hydrophobic amino acids include phenylalanine, tryptophan, tyrosine, alanine, isoleucine, leucine, and valine. In certain embodiments, the amino acid at position 29 according to AHo numbering in the light chain variable region is lysine or arginine. In one particular embodiment, the amino acid at position 29 according to AHo numbering in the light chain variable region is lysine. In related embodiments, the amino acid at position 61 according to AHo numbering in the heavy chain variable region is isoleucine, leucine, valine, glutamine, asparagine, arginine, or lysine. In one embodiment, the amino acid at position 61 according to AHo numbering in the heavy chain variable region is isoleucine. In another embodiment, the amino acid at position 61 according to AHo numbering in the heavy chain variable region is glutamine or asparagine. In yet another embodiment, the amino acid at position 61 according to AHo numbering in the heavy chain variable region is arginine.

In some embodiments, the amino acid at position 66 according to AHo numbering in the heavy chain variable region of the antibody or antigen-binding fragment thereof is a basic or neutral hydrophilic amino acid that interacts with amino acids Asn60 or Ile61 of human PAC1 (SEQ ID NO: 1). The amino acid at position 66 according AHo numbering in the heavy chain variable region may be glutamine, asparagine, arginine, or lysine. In one embodiment, the amino acid at position 66 according AHo numbering in the heavy chain variable region is arginine. In another embodiment, the amino acid at position 66 according AHo numbering in the heavy chain variable region is asparagine.

The paratope-epitope interactions described above were found to correlate with improvements in inhibitory potency with IC50 values in the picomolar range. See Example 3. Thus, antibodies or antigen-binding fragments thereof having the above-specified amino acids at positions 29 in the light chain variable region and positions 61 and/or 66 in the heavy chain variable region according to AHo numbering and interacting with the specified residues in the human PAC1 receptor (Glu120, Asp121, Asn60, and/or Ile61 of SEQ ID NO: 1) would be expected to have enhanced inhibitory potency, e.g. inhibit PACAP-induced activation of human PAC1 with an IC50 less than 500 pM as measured by a cell-based cAMP assay. In some embodiments, such antibodies or antigen-binding fragments may inhibit PACAP-induced activation of human PAC1 with an IC50 less than 300 pM as measured by a cell-based cAMP assay. In these and other embodiments, the antibodies or antigen-binding fragments thereof may have a light chain variable region comprising a sequence that is at least 90% identical to the sequence of SEQ ID NO: 52 and a heavy chain variable region comprising a sequence that is at least 90% identical to the sequence of SEQ ID NO: 191.

The antibodies or antigen-binding fragments of the invention may comprise one or more complementarity determining regions (CDR) from the light and heavy chain variable regions of antibodies that specifically bind to human PAC1 as described herein. The term “CDR” refers to the complementarity determining region (also termed “minimal recognition units” or “hypervariable region”) within antibody variable sequences. There are three heavy chain variable region CDRs (CDRH1, CDRH2 and CDRH3) and three light chain variable region CDRs (CDRL1, CDRL2 and CDRL3). The term “CDR region” as used herein refers to a group of three CDRs that occur in a single variable region (i.e. the three light chain CDRs or the three heavy chain CDRs). The CDRs in each of the two chains typically are aligned by the framework regions (FRs) to form a structure that binds specifically with a specific epitope or domain on the target protein (e.g., human PAC1). From N-terminus to C-terminus, naturally-occurring light and heavy chain variable regions both typically conform with the following order of these elements: FR1, CDR1, FR2, CDR2, FR3, CDR3 and FR4. A numbering system has been devised for assigning numbers to amino acids that occupy positions in each of these domains. This numbering system is defined in Kabat Sequences of Proteins of Immunological Interest (1987 and 1991, NIH, Bethesda, MD), or Chothia & Lesk, 1987, J. Mol. Biol. 196:901-917; Chothia et al., 1989, Nature 342:878-883. Complementarity determining regions (CDRs) and framework regions (FR) of a given antibody may be identified using this system. Other numbering systems for the amino acids in immunoglobulin chains include IMGT® (the international ImMunoGeneTics information system; Lefranc et al., Dev. Comp. Immunol. 29:185-203; 2005) and AHo (Honegger and Pluckthun, J. Mol. Biol. 309(3):657-670; 2001).

In certain embodiments, the anti-PAC1 antibodies or antigen-binding fragments thereof of the invention comprise at least one light chain variable region comprising a CDRL1, CDRL2, and CDRL3, and at least one heavy chain variable region comprising a CDRH1, CDRH2, and CDRH3 from any of the anti-PAC1 antibodies described herein. Light chain and heavy chain variable regions and associated CDRs of exemplary human anti-PAC1 antibodies are set forth below in Tables 1A and 1B, respectively.

TABLE 1A
Exemplary Anti-Human PAC1 Antibody Light Chain Variable Region Amino
Acid Sequences

| | | | | | |
|---|---|---|---|---|---|
| | VL | | | | |
| Ab ID. | Group | VL Amino Acid Sequence | CDRL1 | CDRL2 | CDRL3 |

29G4 variants

| | | | | | |
|---|---|---|---|---|---|
| 29G4v10 | LV-01 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIKR | | | |
| | | (SEQ ID NO: 52) | | | |
| 29G4v22 | LV-02 | DIQLTQSPSFLSASVGDRVTITCRA | RASQSIGRSLH | YASQSLS | HQSSRLPFT (SEQ |
| | | SQSIGRSLHWYQQKPGKAPKLLEK | (SEQ ID NO: 6) | (SEQ ID NO: 26) | ID NO: 36) |
| | | YASQSLSGVPSRFSGSGSGTEFTLT | | | |
| | | ISSLQPEDFATYYCHQSSRLPFTFG | | | |
| | | PGTKVDIKR | | | |
| | | (SEQ ID NO: 53) | | | |
| iPS:420649 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iPS:420653 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iPS:420657 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iPS:420661 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iPS:420665 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iPS:420672 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iPS:420679 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iPS:420686 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iPS:420690 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:420697 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:420704 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:420711 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:420718 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:420725 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:420732 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:420739 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:420746 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:420753 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:420760 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:420767 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:420774 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:420781 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:420788 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:420795 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:420802 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:420809 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:420816 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:420823 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:420830 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:420837 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iPS:420841 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iPS:420845 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iPS:420849 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iP:420853 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iPS:420857 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iPS:420861 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iPS:420865 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iPS:420869 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iPS:420873 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iPS:420877 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iPS:420881 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iPS:420885 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iPS:420889 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iPS:420893 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iPS:420897 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iPS:420901 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:420908 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:420915 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:420922 | LV-04 | EIVLTQSPATLSLSPGERATLSCRA | RASQ SVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:420929 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:420936 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:420943 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:420950 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:420957 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:420964 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:420971 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:420978 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:420985 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:420992 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:420999 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:421006 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:421013 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:421020 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:421027 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iPS:421031 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iPS:421035 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iPS:421039 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iPS:421043 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iPS:421047 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iPS:421051 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iPS:421055 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iPS:421059 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iPS:421063 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iPS:421067 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iPS:421071 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iPS:421075 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iPS:421079 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iPS:421083 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iPS:421087 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iPS:421091 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:421098 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:421105 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:421112 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:421119 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:421126 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:421133 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:421140 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:421147 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iPS:421151 | LV-05 | EIVLTQSPATLSLSPGERATLSCRA | RASQSVWRSLH | YASQSLS | HQSSRLPFT (SEQ |
| | | SQSVWRSLHWYQQKPGQAPRLLI | (SEQ ID NO: 8) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 56) | | | |
| iPS:391478 | LV-06 | EIVLTQSPATLSLSPGERATLSCRA | RASQSVGRNLH | YASQSLS | HQSSRLPFT (SEQ |
| | | SQSVGRNLHWYQQKPGQAPRLLI | (SEQ ID NO: 9) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 57) | | | |
| iPS:421157 | LV-07 | EIVLTQSPATLSLSPGERATLSCRA | RASQSVGRSLH | YASQSLS | HQSSMLPFT (SEQ |
| | | SQSVGRSLHWYQQKPGQAPRLLI | (SEQ ID NO: 5) | (SEQ ID NO: 26) | ID NO: 37) |
| | | KYASQSLSGIPARFSGSGSGTDFTL | | | |
| | | TISSLEPEDFAVYYCHQSSMLPFTF | | | |
| | | GPGTKVDIK | | | |
| | | (SEQ ID NO: 58) | | | |
| iPS:421163 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:391578 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:421170 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:421176 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:421182 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:421189 | LV-08 | EIVLTQSPATLSLSPGERATLSCRA | RASKSVWRSLH | YASQSLS | HQSSRLPFT (SEQ |
| | | SKSVWRSLHWYQQKPGQAPRLLI | (SEQ ID NO: 10) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 59) | | | |
| iPS:421195 | LV-09 | EIVLTQSPATLSLSPGERATLSCRA | RASKSVGRNLH | YASQSLS | HQSSRLPFT (SEQ |
| | | SKSVGRNLHWYQQKPGQAPRLLI | (SEQ ID NO: 11) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 60) | | | |
| iPS:421201 | LV-10 | EIVLTQSPATLSLSPGERATLSCRA | RASKSVGRSLH | YASQSLS | HQSSMLPFT (SEQ |
| | | SKSVGRSLHWYQQKPGQAPRLLI | (SEQ ID NO: 7) | (SEQ ID NO: 26) | ID NO: 37) |
| | | KYASQSLSGIPARFSGSGSGTDFTL | | | |
| | | TISSLEPEDFAVYYCHQSSMLPFTF | | | |
| | | GPGTKVDIK | | | |
| | | (SEQ ID NO: 61) | | | |
| iPS:421207 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iPS:421211 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iPS:421215 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iPS:421219 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iPS:421223 | LV-03 | EIVLTQSPATLSLSPGERATLSCRA | RASKSVGRSLH | YASQSLS | HQSSRLPFT (SEQ |
| | | SKSVGRSLHWYQQKPGQAPRLLI | (SEQ ID NO: 7) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 54) | | | |
| iPS:421227 | LV-05 | EIVLTQSPATLSLSPGERATLSCRA | RASQSVWRSLH | YASQSLS | HQSSRLPFT (SEQ |
| | | SQSVWRSLHWYQQKPGQAPRLLI | (SEQ ID NO: 8) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 56) | | | |
| iPS:421231 | LV-06 | EIVLTQSPATLSLSPGERATLSCRA | RASQSVGRNLH | YASQSLS | HQSSRLPFT (SEQ |
| | | SQSVGRNLHWYQQKPGQAPRLLI | (SEQ ID NO: 9) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 57) | | | |
| iPS:421235 | LV-07 | EIVLTQSPATLSLSPGERATLSCRA | RASQSVGRSLH | YASQSLS | HQSSMLPFT (SEQ |
| | | SQSVGRSLHWYQQKPGQAPRLLI | (SEQ ID NO: 5) | (SEQ ID NO: 26) | ID NO: 37) |
| | | KYASQSLSGIPARFSGSGSGTDFTL | | | |
| | | TISSLEPEDFAVYYCHQSSMLPFTF | | | |
| | | GPGTKVDIK | | | |
| | | (SEQ ID NO: 58) | | | |
| iPS:421239 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:421246 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:421253 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:421260 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:421267 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:421274 | LV-05 | EIVLTQSPATLSLSPGERATLSCRA | RASQSVWRSLH | YASQSLS | HQSSRLPFT (SEQ |
| | | SQSVWRSLHWYQQKPGQAPRLLI | (SEQ ID NO: 8) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 56) | | | |
| iPS:421278 | LV-06 | EIVLTQSPATLSLSPGERATLSCRA | RASQSVGRNLH | YASQSLS | HQSSRLPFT (SEQ |
| | | SQSVGRNLHWYQQKPGQAPRLLI | (SEQ ID NO: 9) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 57) | | | |
| iPS:421282 | LV-07 | EIVLTQSPATLSLSPGERATLSCRA | RASQSVGRSLH | YASQSLS | HQSSMLPFT (SEQ |
| | | SQSVGRSLHWYQQKPGQAPRLLI | (SEQ ID NO: 5) | (SEQ ID NO: 26) | ID NO: 37) |
| | | KYASQSLSGIPARFSGSGSGTDFTL | | | |
| | | TISSLEPEDFAVYYCHQSSMLPFTF | | | |
| | | GPGTKVDIK | | | |
| | | (SEQ ID NO: 58) | | | |
| iPS:421286 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:421293 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:421300 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:421307 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:421314 | LV-05 | EIVLTQSPATLSLSPGERATLSCRA | RASQSVWRSLH | YASQSLS | HQSSRLPFT (SEQ |
| | | SQSVWRSLHWYQQKPGQAPRLLI | (SEQ ID NO: 8) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 56) | | | |
| iPS:421318 | LV-06 | EIVLTQSPATLSLSPGERATLSCRA | RASQSVGRNLH | YASQSLS | HQSSRLPFT (SEQ |
| | | SQSVGRNLHWYQQKPGQAPRLLI | (SEQ ID NO: 9) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 57) | | | |
| iPS:421322 | LV-07 | EIVLTQSPATLSLSPGERATLSCRA | RASQSVGRSLH | YASQSLS | HQSSMLPFT (SEQ |
| | | SQSVGRSLHWYQQKPGQAPRLLI | (SEQ ID NO: 5) | (SEQ ID NO: 26) | ID NO: 37) |
| | | KYASQSLSGIPARFSGSGSGTDFTL | | | |
| | | TISSLEPEDFAVYYCHQSSMLPFTF | | | |
| | | GPGTKVDIK | | | |
| | | (SEQ ID NO: 58) | | | |
| iPS:421326 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:421333 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:421340 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:421347 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:421354 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:421361 | LV-05 | EIVLTQSPATLSLSPGERATLSCRA | RASQSVWRSLH | YASQSLS | HQSSRLPFT (SEQ |
| | | SQSVWRSLHWYQQKPGQAPRLLI | (SEQ ID NO: 8) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 56) | | | |
| iPS:421365 | LV-06 | EIVLTQSPATLSLSPGERATLSCRA | RASQSVGRNLH | YASQSLS | HQSSRLPFT (SEQ |
| | | SQSVGRNLHWYQQKPGQAPRLLI | (SEQ ID NO: 9) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 57) | | | |
| iPS:421369 | LV-07 | EIVLTQSPATLSLSPGERATLSCRA | RASQSVGRSLH | YASQSLS | HQSSMLPFT (SEQ |
| | | SQSVGRSLHWYQQKPGQAPRLLI | (SEQ ID NO: 5) | (SEQ ID NO: 26) | ID NO: 37) |
| | | KYASQSLSGIPARFSGSGSGTDFTL | | | |
| | | TISSLEPEDFAVYYCHQSSMLPFTF | | | |
| | | GPGTKVDIK | | | |
| | | (SEQ ID NO: 58) | | | |
| iPS:421373 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:421380 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:421387 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:421394 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:421855 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:421861 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:421867 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:421873 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:421879 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:421885 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:421891 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:421897 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:421903 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:421909 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:421915 | LV-04 | EIVLTQSPATLSLSPGERATLSCRA | RASQSVGRSLH | YASQSLS | HQSSRLPFT (SEQ |
| | | SQSVGRSLHWYQQKPGQAPRLLI | (SEQ ID NO: 5) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIK | | | |
| | | (SEQ ID NO: 55) | | | |
| iPS:480711 | LV-11 | EIVLTQSPATLSLSPGERATLSCRA | RASKSVGWSLH | YASQSLS | HQSSRLPFT (SEQ |
| | | SKSVGWSLHWYQQKPGQAPRLLI | (SEQ ID NO: 12) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIKR | | | |
| | | (SEQ ID NO: 62) | | | |
| iPS:480706 | LV-11 | EIVLTQSPATLSLSPGERATLSCRA | RASKSVGWSLH | YASQSLS | HQSSRLPFT (SEQ |
| | | SKSVGWSLHWYQQKPGQAPRLLI | (SEQ ID NO: 12) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIKR | | | |
| | | (SEQ ID NO: 62) | | | |
| iPS:480713 | LV-12 | EIVLTQSPATLSLSPGERATLSCRA | RASKSVGYSLH | YASQSLS | HQSSRLPFT (SEQ |
| | | SKSVGYSLHWYQQKPGQAPRLLI | (SEQ ID NO: 13) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIKR | | | |
| | | (SEQ ID NO: 63) | | | |
| iPS:480705 | LV-11 | EIVLTQSPATLSLSPGERATLSCRA | RASKSVGWSLH | YASQSLS | HQSSRLPFT (SEQ |
| | | SKSVGWSLHWYQQKPGQAPRLLI | (SEQ ID NO: 12) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIKR | | | |
| | | (SEQ ID NO: 62) | | | |
| iPS:480707 | LV-11 | EIVLTQSPATLSLSPGERATLSCRA | RASKSVGWSLH | YASQSLS | HQSSRLPFT (SEQ |
| | | SKSVGWSLHWYQQKPGQAPRLLI | (SEQ ID NO: 12) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIKR | | | |
| | | (SEQ ID NO: 62) | | | |
| iPS:480708 | LV-11 | EIVLTQSPATLSLSPGERATLSCRA | RASKSVGWSLH | YASQSLS | HQSSRLPFT (SEQ |
| | | SKSVGWSLHWYQQKPGQAPRLLI | (SEQ ID NO: 12) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIKR | | | |
| | | (SEQ ID NO: 62) | | | |
| iPS:480709 | LV-13 | EIVLTQSPATLSLSPGERATLSCRA | RASKAVGWSLH | YASQSLS | HQSSRLPFT (SEQ |
| | | SKAVGWSLHWYQQKPGQAPRLLI | (SEQ ID NO: 14) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIKR | | | |
| | | (SEQ ID NO: 64) | | | |
| iPS:480712 | LV-11 | EIVLTQSPATLSLSPGERATLSCRA | RASKSVGWSLH | YASQSLS | HQSSRLPFT (SEQ |
| | | SKSVGWSLHWYQQKPGQAPRLLI | (SEQ ID NO: 12) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIKR | | | |
| | | (SEQ ID NO: 62) | | | |
| iPS:480704 | LV-11 | EIVLTQSPATLSLSPGERATLSCRA | RASKSVGWSLH | YASQSLS | HQSSRLPFT (SEQ |
| | | SKSVGWSLHWYQQKPGQAPRLLI | (SEQ ID NO: 12) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIKR | | | |
| | | (SEQ ID NO: 62) | | | |
| iPS:480710 | LV-11 | EIVLTQSPATLSLSPGERATLSCRA | RASKSVGWSLH | YASQSLS | HQSSRLPFT (SEQ |
| | | SKSVGWSLHWYQQKPGQAPRLLI | (SEQ ID NO: 12) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIKR | | | |
| | | (SEQ ID NO: 62) | | | |
| iPS:480716 | LV-14 | EIVLTQSPATLSLSPGERATLSCRA | RASKSVGQSLH | YASQSLS | HQSSRLPFT (SEQ |
| | | SKSVGQSLHWYQQKPGQAPRLLI | (SEQ ID NO: 15) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIKR | | | |
| | | (SEQ ID NO: 65) | | | |
| iPS:480715 | LV-14 | EIVLTQSPATLSLSPGERATLSCRA | RASKSVGQSLH | YASQSLS | HQSSRLPFT (SEQ |
| | | SKSVGQSLHWYQQKPGQAPRLLI | (SEQ ID NO: 15) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIKR | | | |
| | | (SEQ ID NO: 65) | | | |
| iPS:480717 | LV-14 | EIVLTQSPATLSLSPGERATLSCRA | RASKSVGQSLH | YASQSLS | HQSSRLPFT (SEQ |
| | | SKSVGQSLHWYQQKPGQAPRLLI | (SEQ ID NO: 15) | (SEQ ID NO: 26) | ID NO: 36) |
| | | KYASQSLSGIPARFSGSGSGTDFTL | | | |
| | | TISSLEPEDFAVYYCHQSSRLPFTF | | | |
| | | GPGTKVDIKR | | | |
| | | (SEQ ID NO: 65) | | | |
| iPS:480714 | LV-15 | EIVLTQSPATLSLSPGERATLSCRA | RASRSVGLALH | YASQSLS | HQSSFLPFT (SEQ |
| | | SRSVGLALHWYQQKPGQAPRLLI | (SEQ ID NO: 16) | (SEQ ID NO: 26) | ID NO: 38) |
| | | KYASQSLSGIPARFSGSGSGTDFTL | | | |
| | | TISSLEPEDFAVYYCHQSSFLPFTF | | | |
| | | GPGTKVDIKR | | | |
| | | (SEQ ID NO: 66) | | | |

19H8 variants

| | | | | | |
|---|---|---|---|---|---|
| 19H8 | LV-16 | DIQMTQSPSSLSASVGDRITITCRA | RASQSISRYLN | AASSLQS | QQSYSPPFT (SEQ |
| | | SQSISRYLNWYQQKPGKAPKLLIY | (SEQ ID NO: 17) | (SEQ ID NO: 27) | ID NO: 39) |
| | | AASSLQSGIPSRFSGSGSGTDFTLTI | | | |
| | | NSLQPEDFATYFCQQSYSPPFTFGP | | | |
| | | GTKVDIKR | | | |
| | | (SEQ ID NO: 67) | | | |
| iPS:448202 | LV-17 | DIQMTQSPSSLSASVGDRITITCRA | RASQSISRYLN | AGQRLQS | QQAIGMPYT |
| | | SQSISRYLNWYQQKPGKAPKLLIF | (SEQ ID NO: 17) | (SEQ ID NO: 28) | (SEQ ID NO: 40) |
| | | AGQRLQSGIPSRFSGSGSGTDFTLT | | | |
| | | INSLQPEDFATYFCQQAIG1VIPYTF | | | |
| | | GPGTKVDIK | | | |
| | | (SEQ ID NO: 68) | | | |
| iPS:449375 | LV-18 | DIQMTQSPSSLSASVGDRITITCRA | RASQYIVRYLN | AAHHLQS | QQAIQEPYT (SEQ |
| | | SQYIVRYLNWYQQKPGKAPKLLI | (SEQ ID NO: 18) | (SEQ ID NO: 29) | ID NO: 41) |
| | | YAAHHLQSGIPSRFSGSGSGTDFT | | | |
| | | LTINSLQPEDFATYFCQQAIQEPYT | | | |
| | | FGPGTKVDIK | | | |
| | | (SEQ ID NO: 69) | | | |
| iPS:448083 | LV-19 | DIQMTQSPSSLSASVGDRITITCRA | RASQTIVRYLN | AGQRLQS | QQAIINPYT (SEQ |
| | | SQTIVRYLNWYQQKPGKAPKLLIF | (SEQ ID NO: 19) | (SEQ ID NO: 28) | ID NO: 42) |
| | | AGQRLQSGIPSRFSGSGSGTDFTLT | | | |
| | | INSLQPEDFATYFCQQAIINPYTFG | | | |
| | | PGTKVDIK | | | |
| | | (SEQ ID NO: 70) | | | |
| iPS:452128 | LV-20 | DIQMTQSPSSLSASVGDRITITCRA | RASQYIVRYLN | AANMLQS | QQAINQPYT |
| | | SQYIVRYLNWYQQKPGKAPKLLI | (SEQ ID NO: 18) | (SEQ ID NO: 30) | (SEQ ID NO: 43) |
| | | YAANMLQSGIPSRFSGSGSGTDFT | | | |
| | | LTINSLQPEDFATYFCQQAINQPYT | | | |
| | | FGPGTKVDIK | | | |
| | | (SEQ ID NO: 71) | | | |
| iPS:448195 | LV-21 | DIQMTQSPSSLSASVGDRITITCRA | RASQKIARYLV | AANMLQS | QQSIQQPYT (SEQ |
| | | SQKIARYLVWYQQKPGKAPKLLI | (SEQ ID NO: 20) | (SEQ ID NO: 30) | ID NO: 44) |
| | | YAANMLQSGIPSRFSGSGSGTDFT | | | |
| | | LTINSLQPEDFATYFCQQSIQQPYT | | | |
| | | FGPGTKVDIK | | | |
| | | (SEQ ID NO: 72) | | | |
| iPS:448466 | LV-22 | DIQMTQSPSSLSASVGDRITITCRA | RASQSISRYLN | AGQRLQS | QQAIQQPYT |
| | | SQSISRYLNWYQQKPGKAPKLLIF | (SEQ ID NO: 17) | (SEQ ID NO: 28) | (SEQ ID NO: 45) |
| | | AGQRLQSGIPSRFSGSGSGTDFTLT | | | |
| | | INSLQPEDFATYFCQQAIQQPYTFG | | | |
| | | PGTKVDIKR | | | |
| | | (SEQ ID NO: 73) | | | |
| iPS:448689 | LV-23 | DIQMTQSPSSLSASVGDRITITCRA | RASQYIVRYLN | ASYNLQS | QQAIMAPYT |
| | | SQYIVRYLNWYQQKPGKAPKLLI | (SEQ ID NO: 18) | (SEQ ID NO: 31) | (SEQ ID NO: 46) |
| | | YASYNLQSGIPSRFSGSGSGTDFTL | | | |
| | | TINSLQPEDFATYFCQQAIMAPYT | | | |
| | | FGPGTKVDIK | | | |
| | | (SEQ ID NO: 74) | | | |
| iPS:449034 | LV-24 | DIQMTQSPSSLSASVGDRITITCRA | RASQPIAQYLN | AGRYLQS | QQAIQNPYT |
| | | SQPIAQYLNWYQQKPGKAPKLLIY | (SEQ ID NO: 21) | (SEQ ID NO: 32) | (SEQ ID NO: 47) |
| | | AGRYLQSGIPSRFSGSGSGTDFTLT | | | |
| | | INSLQPEDFATYFCQQAIQNPYTFG | | | |
| | | PGTKVDIK | | | |
| | | (SEQ ID NO: 75) | | | |
| iPS:448075 | LV-25 | DIQMTQSPSSLSASVGDRITITCRA | RASQSISRYLN | AGQRLQS | QQAIVQPYT |
| | | SQSISRYLNWYQQKPGKAPKLLIF | (SEQ ID NO: 17) | (SEQ ID NO: 28) | (SEQ ID NO: 48) |
| | | AGQRLQSGIPSRFSGSGSGTDFTLT | | | |
| | | INSLQPEDFATYFCQQAIVQPYTFG | | | |
| | | PGTKVDIK | | | |
| | | (SEQ ID NO: 76) | | | |
| iPS:448924 | LV-26 | DIQMTQSPSSLSASVGDRITITCRA | RASQPISRYLS | AGQRLQS | QQAISIPYT (SEQ |
| | | SQPISRYLSWYQQKPGKAPKLLIF | (SEQ ID NO: 22) | (SEQ ID NO: 28) | ID NO: 49) |
| | | AGQRLQSGIPSRFSGSGSGTDFTLT | | | |
| | | INSLQPEDFATYFCQQAISIPYTFGP | | | |
| | | GTKVDIK | | | |
| | | (SEQ ID NO: 77) | | | |
| iPS:448752 | LV-27 | DIQMTQSPSSLSASVGDRITITCRA | RASQQIARYLN | ASYNLQS | QQAIIQPYT (SEQ |
| | | SQQIARYLNWYQQKPGKAPKLLI | (SEQ ID NO: 23) | (SEQ ID NO: 31) | ID NO: 50) |
| | | YASYNLQSGIPSRFSGSGSGTDFTL | | | |
| | | TINSLQPEDFATYFCQQAIIQPYTF | | | |
| | | GPGTKVDIK | | | |
| | | (SEQ ID NO: 78) | | | |
| iPS:448772 | LV-28 | DIQMTQSPSSLSASVGDRITITCRA | RASQSISRYLN | ASYNLQS | QQAIQNPYT |
| | | SQSISRYLNWYQQKPGKAPKLLIY | (SEQ ID NO: 17) | (SEQ ID NO: 31) | (SEQ ID NO: 47) |
| | | ASYNLQSGIPSRFSGSGSGTDFTLT | | | |
| | | INSLQPEDFATYFCQQAIQNPYTFG | | | |
| | | PGTKVDIK | | | |
| | | (SEQ ID NO: 79) | | | |
| iPS:448117 | LV-29 | DIQMTQSPSSLSASVGDRITITCRA | RASQTIVRYLN | AGQRLQS | QQSIQTPYT (SEQ |
| | | SQTIVRYLNWYQQKPGKAPKLLIF | (SEQ ID NO: 19) | (SEQ ID NO: 28) | ID NO: 51) |
| | | AGQRLQSGIPSRFSGSGSGTDFTLT | | | |
| | | INSLQPEDFATYFCQQSIQTPYTFG | | | |
| | | PGTKVDIK | | | |
| | | (SEQ ID NO: 80) | | | |
| iPS:448788 | LV-30 | DIQMTQSPSSLSASVGDRITITCRA | RASQSISRYLN | AGRILQS | QQAIINPYT (SEQ |
| | | SQSISRYLNWYQQKPGKAPKLLIY | (SEQ ID NO: 17) | (SEQ ID NO: 33) | ID NO: 42) |
| | | AGRILQSGIPSRFSGSGSGTDFTLTI | | | |
| | | NSLQPEDFATYFCQQAIINPYTFGP | | | |
| | | GTKVDIK | | | |
| | | (SEQ ID NO: 81) | | | |
| iPS:448593 | LV-28 | DIQMTQSPSSLSASVGDRITITCRA | RASQSISRYLN | ASYNLQS | QQAIQNPYT |
| | | SQSISRYLNWYQQKPGKAPKLLIY | (SEQ ID NO: 17) | (SEQ ID NO: 31) | (SEQ ID NO: 47) |
| | | ASYNLQSGIPSRFSGSGSGTDFTLT | | | |
| | | INSLQPEDFATYFCQQAIQNPYTFG | | | |
| | | PGTKVDIK | | | |
| | | (SEQ ID NO: 79) | | | |
| iPS:448238 | LV-31 | DIQMTQSPSSLSASVGDRITITCRA | RASQRIARYLN | AGSILQS | QQAIQNPYT |
| | | SQRIARYLNWYQQKPGKAPKLLIF | (SEQ ID NO: 24) | (SEQ ID NO: 34) | (SEQ ID NO: 47) |
| | | AGSILQSGIPSRFSGSGSGTDFTLTI | | | |
| | | NSLQPEDFATYFCQQAIQNPYTFG | | | |
| | | PGTKVDIK | | | |
| | | (SEQ ID NO: 82) | | | |
| iPS:448901 | LV-32 | DIQMTQSPSSLSASVGDRITITCRA | RASQSISRYLN | ASYNLQS | QQSIQQPYT (SEQ |
| | | SQSISRYLNWYQQKPGKAPKLLIY | (SEQ ID NO: 17) | (SEQ ID NO: 31) | ID NO: 44) |
| | | ASYNLQSGIPSRFSGSGSGTDFTLT | | | |
| | | INSLQPEDFATYFCQQSIQQPYTFG | | | |
| | | PGTKVDIK | | | |
| | | (SEQ ID NO: 83) | | | |
| iPS:448655 | LV-33 | DIQMTQSPSSLSASVGDRITITCRA | RASQYIVRYLN | ASYNLQS | QQAIQQPYT |
| | | SQYIVRYLNWYQQKPGKAPKLLI | (SEQ ID NO: 18) | (SEQ ID NO: 31) | (SEQ ID NO: 45) |
| | | YASYNLQSGIPSRFSGSGSGTDFTL | | | |
| | | TINSLQPEDFATYFCQQAIQQPYTF | | | |
| | | GPGTKVDIK | | | |
| | | (SEQ ID NO: 84) | | | |
| iPS:448730 | LV-34 | DIQMTQSPSSLSASVGDRITITCRA | RASQMIARYLN | ASYNLQS | QQAIINPYT (SEQ |
| | | SQMIARYLNWYQQKPGKAPKLLI | (SEQ ID NO: 25) | (SEQ ID NO: 31) | ID NO: 42) |
| | | YASYNLQSGIPSRFSGSGSGTDFTL | | | |
| | | TINSLQPEDFATYFCQQAIINPYTF | | | |
| | | GPGTKVDIK | | | |
| | | (SEQ ID NO: 85) | | | |
| iPS:449027 | LV-35 | DIQMTQSPSSLSASVGDRITITCRA | RASQYIVRYLN | GARNLQS | QQSIQTPYT (SEQ |
| | | SQYIVRYLNWYQQKPGKAPKLLI | (SEQ ID NO: 18) | (SEQ ID NO: 35) | ID NO: 51) |
| | | YGARNLQSGIPSRFSGSGSGTDFTL | | | |
| | | TINSLQPEDFATYFCQQSIQTPYTF | | | |
| | | GPGTKVDIK | | | |
| | | (SEQ ID NO: 86) | | | |
| 3574 | LV-36 | DIQMTQSPSSLSASVGDRITITCRA | RASQSISRYLN | AASSLQS | QQSYSPPFT (SEQ |
| | | SQSISRYLNWYQQKPGKAPKLLIY | (SEQ ID NO: 17) | (SEQ ID NO: 27) | ID NO: 39) |
| | | AASSLQSGIPSRFSGSGSGTDFTLTI | | | |
| | | NSLQPEDFATYFCQQSYSPPFTFGP | | | |
| | | GTKVDIK | | | |
| | | (SEQ ID NO: 87) | | | |
| 3575 | LV-27 | DIQMTQSPSSLSASVGDRITITCRA | RASQQIARYLN | ASYNLQS | QQAIIQPYT (SEQ |
| | | SQQIARYLNWYQQKPGKAPKLLI | (SEQ ID NO: 23) | (SEQ ID NO: 31) | ID NO: 50) |
| | | YASYNLQSGIPSRFSGSGSGTDFTL | | | |
| | | TINSLQPEDFATYFCQQAIIQPYTF | | | |
| | | GPGTKVDIK | | | |
| | | (SEQ ID NO: 78) | | | |

TABLE 1B
Exemplary Anti-Human PAC1 Antibody Heavy Chain Variable Region Amino
Acid Sequences

| | | | | | |
|---|---|---|---|---|---|
| | VH | | | | |
| Ab ID. | Group | VH Amino Acid Sequence | CDRH1 | CDRH2 | CDRH3 |

29G4 variants

| | | | | | |
|---|---|---|---|---|---|
| 29G4v10 | HV-01 | QVQLVESGGGVVQPGRSLR | RFAMH | VISYDGGNKYYAESVKG | GYDVLTGYPDY |
| | | LSCAASGFTFSRFAMHWVR | (SEQ ID | (SEQ ID NO: 106) | (SEQ ID NO: 171) |
| | | QAPGKGLEWVAVISYDGGN | NO: 88) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 191) | | | |
| 29G4v22 | HV-02 | QVQLVESGAEVVKPGASVK | RFAMH | VISYDGGNKYYAESVKG | GYDVLTGYPDY |
| | | VSCKASGFTESRFAMHWVR | (SEQ ID | (SEQ ID NO: 106) | (SEQ ID NO: 171) |
| | | QAPGQGLEWMGVISYDGGN | NO: 88) | | |
| | | KYYAESVKGRVTMTRDTST | | | |
| | | STLYMELSSLRSEDTAVYYC | | | |
| | | ARGYDVLTGYPDYWGQGTL | | | |
| | | VTVSS (SEQ ID NO: 192) | | | |
| iPS:420649 | HV-03 | QVQLVESGGGVVQPGRSLR | RFAMH | VISYNGGNKYYAESVKG | GYDVLTGYPDY |
| | | LSCAASGFTFSRFAMHWVR | (SEQ ID | (SEQ ID NO: 107) | (SEQ ID NO: 171) |
| | | QAPGKGLEWVAVISYNGGN | NO: 88) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 193) | | | |
| iPS:420653 | HV-04 | QVQLVESGGGVVQPGRSLR | RFAMH | VISYIGGNKYYAESVKG | GYDVLTGYPDY |
| | | LSCAASGFTFSRFAMHWVR | (SEQ ID | (SEQ ID NO: 108) | (SEQ ID NO: 171) |
| | | QAPGKGLEWVAVISYIGGN | NO: 88) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 194) | | | |
| iPS:420657 | HV-05 | QVQLVESGGGVVQPGRSLR | RFAMH | VISYQGGNKYYAESVKG | GYDVLTGYPDY |
| | | LSCAASGFTFSRFAMHWVR | (SEQ ID | (SEQ ID NO: 109) | (SEQ ID NO: 171) |
| | | QAPGKGLEWVAVISYQGGN | NO: 88) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 195) | | | |
| iPS:420661 | HV-06 | QVQLVESGGGVVQPGRSLR | RFAMH | VISYYGGNKYYAESVKG | GYDVLTGYPDY |
| | | LSCAASGFTFSRFAMHWVR | (SEQ ID | (SEQ ID NO: 110) | (SEQ ID NO: 171) |
| | | QAPGKGLEWVAVISYYGGN | NO: 88) | | |
| | | KYYAESVKGRFTISRDNSKN | | | |
| | | TLYLQMNSLRAEDTALFYC | | | |
| | | ARGYDVLTGYPDYWGQGTL | | | |
| | | VVTVSS (SEQ ID NO: 196) | | | |
| iPS:420665 | HV-07 | QVQLVESGGGVVQPGRSLR | RFAMH | VISYDGRNKYYAESVKG | GYDVLTGYPDY |
| | | LSCAASGFTFSRFAMHWVR | (SEQ ID | (SEQ ID NO: 111) | (SEQ ID NO: 171) |
| | | QAPGKGLEWVAVISYDGRN | NO: 88) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 197) | | | |
| iPS:420672 | HV-08 | QVQLVESGGGVVQPGRSLR | RFAMH | VISYDGNNKYYAESVKG | GYDVLTGYPDY |
| | | LSCAASGFTFSRFAMHWVR | (SEQ ID | (SEQ ID NO: 112) | (SEQ ID NO: 171) |
| | | QAPGKGLEWVAVISYDGNN | NO: 88) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 198) | | | |
| iPS:420679 | HV-09 | QVQLVESGGGVVQPGRSLR | RFAMH | VISYDGGNKYYARSVKG | GYDVLTGYPDY |
| | | LSCAASGFTFSRFAMHWVR | (SEQ ID | (SEQ ID NO: 113) | (SEQ ID NO: 171) |
| | | QAPGKGLEWVAVISYDGGN | NO: 88) | | |
| | | KYYARSVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 199) | | | |
| iPS:420686 | HV-10 | QVQLVESGGGVVQPGRSLR | RFAMH | VISYDGGNKYYAESVKG | GYDILTGYPDY |
| | | LSCAASGFTFSRFAMHWVR | (SEQ ID | (SEQ ID NO: 106) | (SEQ ID NO: 172) |
| | | QAPGKGLEWVAVISYDGGN | NO: 88) | | |
| | | KYYAESVKGRFTISRDNSKN | | | |
| | | TLYLQMNSLRAEDTALFYC | | | |
| | | ARGYDILTGYPDYWGQGTL | | | |
| | | VTVSS (SEQ ID NO: 200) | | | |
| iPS:420690 | HV-11 | QVQLVESGGGVVQPGRSLR | RFAMH | VISYNGRNKYYAESVKG | GYDVLTGYPDY |
| | | LSCAASGFTFSRFAMHWVR | (SEQ ID | (SEQ ID NO: 114) | (SEQ ID NO: 171) |
| | | QAPGKGLEWVAVISYNGRN | NO: 88) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 201) | | | |
| iPS:420697 | HV-12 | QVQLVESGGGVVQPGRSLR | RFAMH | VISYIGRNKYYAESVKG | GYDVLTGYPDY |
| | | LSCAASGFTFSRFAMHWVR | (SEQ ID | (SEQ ID NO: 115) | (SEQ ID NO: 171) |
| | | QAPGKGLEWVAVISYIGRNK | NO: 88) | | |
| | | YYAESVKGRFTISRDNSKNT | | | |
| | | LYLQMNSLRAEDTALFYCA | | | |
| | | RGYDVLTGYPDYWGQGTLV | | | |
| | | TVSS (SEQ ID NO: 202) | | | |
| iPS:420704 | HV-13 | QVQLVESGGGVVQPGRSLR | RFAMH | VISYQGRNKYYAESVKG | GYDVLTGYPDY |
| | | LSCAASGFTFSRFAMHWVR | (SEQ ID | (SEQ ID NO: 116) | (SEQ ID NO: 171) |
| | | QAPGKGLEWVAVISYQGRN | NO: 88) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 203) | | | |
| iPS:420711 | HV-14 | QVQLVESGGGVVQPGRSLR | RFAMH | VISYYGRNKYYAESVKG | GYDVLTGYPDY |
| | | LSCAASGFTFSRFAMHWVR | (SEQ ID | (SEQ ID NO: 117) | (SEQ ID NO: 171) |
| | | QAPGKGLEWVAVISYYGRN | NO: 88) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 204) | | | |
| iPS:420718 | HV-15 | QVQLVESGGGVVQPGRSLR | RFAMH | VISYNGNNKYYAESVKG | GYDVLTGYPDY |
| | | LSCAASGFTFSRFAMHWVR | (SEQ ID | (SEQ ID NO: 118) | (SEQ ID NO: 171) |
| | | QAPGKGLEWVAVISYNGNN | NO: 88) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 205) | | | |
| iPS:420725 | HV-16 | QVQLVESGGGVVQPGRSLR | RFAMH | VISYIGNNKYYAESVKG | GYDVLTGYPDY |
| | | LSCAASGFTFSRFAMHWVR | (SEQ ID | (SEQ ID NO: 119) | (SEQ ID NO: 171) |
| | | QAPGKGLEWVAVISYIGNN | NO: 88) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 206) | | | |
| iPS:420732 | HV-17 | QVQLVESGGGVVQPGRSLR | RFAMH | VISYQGNNKYYAESVKG | GYDVLTGYPDY |
| | | LSCAASGFTFSRFAMHWVR | (SEQ ID | (SEQ ID NO: 120) | (SEQ ID NO: 171) |
| | | QAPGKGLEWVAVISYQGNN | NO: 88) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 207) | | | |
| iPS:420739 | HV-18 | QVQLVESGGGVVQPGRSLR | RFAMH | VISYYGNNKYYAESVKG | GYDVLTGYPDY |
| | | LSCAASGFTFSRFAMHWVR | (SEQ ID | (SEQ ID NO: 121) | (SEQ ID NO: 171) |
| | | QAPGKGLEWVAVISYYGNN | NO: 88) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 208) | | | |
| iPS:420746 | HV-19 | QVQLVESGGGVVQPGRSLR | RFAMH | VISYNGGNKYYARSVKG | GYDVLTGYPDY |
| | | LSCAASGFTFSRFAMHWVR | (SEQ ID | (SEQ ID NO: 122) | (SEQ ID NO: 171) |
| | | QAPGKGLEWVAVISYNGGN | NO: 88) | | |
| | | KYYARSVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 209) | | | |
| iPS:420753 | HV-20 | QVQLVESGGGVVQPGRSLR | RFAMH | VISYIGGNKYYARSVKG | GYDVLTGYPDY |
| | | LSCAASGFTFSRFAMHWVR | (SEQ ID | (SEQ ID NO: 123) | (SEQ ID NO: 171) |
| | | QAPGKGLEWVAVISYIGGN | NO: 88) | | |
| | | KYYARSVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 210) | | | |
| iPS:420760 | HV-21 | QVQLVESGGGVVQPGRSLR | RFAMH | VISYQGGNKYYARSVKG | GYDVLTGYPDY |
| | | LSCAASGFTFSRFAMHWVR | (SEQ ID | (SEQ ID NO: 124) | (SEQ ID NO: 171) |
| | | QAPGKGLEWVAVISYQGGN | NO: 88) | | |
| | | KYYARSVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 211) | | | |
| iPS:420767 | HV-22 | QVQLVESGGGVVQPGRSLR | RFAMH | VISYYGGNKYYARSVKG | GYDVLTGYPDY |
| | | LSCAASGFTFSRFAMHWVR | (SEQ ID | (SEQ ID NO: 125) | (SEQ ID NO: 171) |
| | | QAPGKGLEWVAVISYYGGN | NO: 88) | | |
| | | KYYARSVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 212) | | | |
| iPS:420774 | HV-23 | QVQLVESGGGVVQPGRSLR | RFAMH | VISYNGGNKYYAESVKG | GYDILTGYPDY |
| | | LSCAASGFTFSRFAMHWVR | (SEQ ID | (SEQ ID NO: 107) | (SEQ ID NO: 172) |
| | | QAPGKGLEWVAVISYNGGN | NO: 88) | | |
| | | KYYAESVKGRFTISRDNSKN | | | |
| | | TLYLQMNSLRAEDTALFYC | | | |
| | | ARGYDILTGYPDYWGQGTL | | | |
| | | VTVSS (SEQ ID NO: 213) | | | |
| iPS:420781 | HV-24 | QVQLVESGGGVVQPGRSLR | RFAMH | VISYIGGNKYYAESVKG | GYDILTGYPDY |
| | | LSCAASGFTFSRFAMHWVR | (SEQ ID | (SEQ ID NO: 108) | (SEQ ID NO: 172) |
| | | QAPGKGLEWVAVISYIGGN | NO: 88) | | |
| | | KYYAESVKGRFTISRDNSKN | | | |
| | | TLYLQMNSLRAEDTALFYC | | | |
| | | ARGYDILTGYPDYWGQGTL | | | |
| | | VTVSS (SEQ ID NO: 214) | | | |
| iPS:420788 | HV-25 | QVQLVESGGGVVQPGRSLR | RFAMH | VISYQGGNKYYAESVKG | GYDILTGYPDY |
| | | LSCAASGFTFSRFAMHWVR | (SEQ ID | (SEQ ID NO: 109) | (SEQ ID NO: 172) |
| | | QAPGKGLEWVAVISYQGGN | NO: 88) | | |
| | | KYYAESVKGRFTISRDNSKN | | | |
| | | TLYLQMNSLRAEDTALFYC | | | |
| | | ARGYDILTGYPDYWGQGTL | | | |
| | | VTVSS (SEQ ID NO: 215) | | | |
| iPS:420795 | HV-26 | QVQLVESGGGVVQPGRSLR | RFAMH | VISYYGGNKYYAESVKG | GYDILTGYPDY |
| | | LSCAASGFTFSRFAMHWVR | (SEQ ID | (SEQ ID NO: 110) | (SEQ ID NO: 172) |
| | | QAPGKGLEWVAVISYYGGN | NO: 88) | | |
| | | KYYAESVKGRFTISRDNSKN | | | |
| | | TLYLQMNSLRAEDTALFYC | | | |
| | | ARGYDILTGYPDYWGQGTL | | | |
| | | VTVSS (SEQ ID NO: 216) | | | |
| iPS:420802 | HV-27 | QVQLVESGGGVVQPGRSLR | RFAMH | VISYDGRNKYYARSVKG | GYDVLTGYPDY |
| | | LSCAASGFTFSRFAMHWVR | (SEQ ID | (SEQ ID NO: 126) | (SEQ ID NO: 171) |
| | | QAPGKGLEWVAVISYDGRN | NO: 88) | | |
| | | KYYARSVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 217) | | | |
| iPS:420809 | HV-28 | QVQLVESGGGVVQPGRSLR | RFAMH | VISYDGNNKYYARSVKG | GYDVLTGYPDY |
| | | LSCAASGFTFSRFAMHWVR | (SEQ ID | (SEQ ID NO: 127) | (SEQ ID NO: 171) |
| | | QAPGKGLEWVAVISYDGNN | NO: 88) | | |
| | | KYYARSVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 218) | | | |
| iPS:420816 | HV-29 | QVQLVESGGGVVQPGRSLR | RFAMH | VISYDGRNKYYAESVKG | GYDILTGYPDY |
| | | LSCAASGFTFSRFAMHWVR | (SEQ ID | (SEQ ID NO: 111) | (SEQ ID NO: 172) |
| | | QAPGKGLEWVAVISYDGRN | NO: 88) | | |
| | | KYYAESVKGRFTISRDNSKN | | | |
| | | TLYLQMNSLRAEDTALFYC | | | |
| | | ARGYDILTGYPDYWGQGTL | | | |
| | | VTVSS (SEQ ID NO: 219) | | | |
| iPS:420823 | HV-30 | QVQLVESGGGVVQPGRSLR | RFAMH | VISYDGNNKYYAESVKG | GYDILTGYPDY |
| | | LSCAASGFTFSRFAMHWVR | (SEQ ID | (SEQ ID NO: 112) | (SEQ ID NO: 172) |
| | | QAPGKGLEWVAVISYDGNN | NO: 88) | | |
| | | KYYAESVKGRFTISRDNSKN | | | |
| | | TLYLQMNSLRAEDTALFYC | | | |
| | | ARGYDILTGYPDYWGQGTL | | | |
| | | VTVSS (SEQ ID NO: 220) | | | |
| IPS:420830 | HV-31 | QVQLVESGGGVVQPGRSLR | RFAMH | VISYDGGNKYYARSVKG | GYDILTGYPDY |
| | | LSCAASGFTFSRFAMHWVR | (SEQ ID | (SEQ ID NO: 113) | (SEQ ID NO: 172) |
| | | QAPGKGLEWVAVISYDGGN | NO: 88) | | |
| | | KYYARSVKGRFTISRDNSKN | | | |
| | | TLYLQMNSLRAEDTALFYC | | | |
| | | ARGYDILTGYPDYWGQGTL | | | |
| | | VTVSS (SEQ ID NO: 221) | | | |
| IPS:420837 | HV-11 | QVQLVESGGGVVQPGRSLR | RFAMH | VISYNGRNKYYAESVKG | GYDVLTGYPDY |
| | | LSCAASGFTFSRFAMHWVR | (SEQ ID | (SEQ ID NO: 114) | (SEQ ID NO: 171) |
| | | QAPGKGLEWVAVISYNGRN | NO: 88) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 201) | | | |
| IPS:420841 | HV-12 | QVQLVESGGGVVQPGRSLR | RFAMH | VISYIGRNKYYAESVKG | GYDVLTGYPDY |
| | | LSCAASGFTFSRFAMHWVR | (SEQ ID | (SEQ ID NO: 115) | (SEQ ID NO: 171) |
| | | QAPGKGLEWVAVISYIGRNK | NO: 88) | | |
| | | YYAESVKGRFTISRDNSKNT | | | |
| | | LYLQMNSLRAEDTALFYCA | | | |
| | | RGYDVLTGYPDYWGQGTLV | | | |
| | | TVSS (SEQ ID NO: 202) | | | |
| IPS:420845 | HV-13 | QVQLVESGGGVVQPGRSLR | RFAMH | VISYQGRNKYYAESVKG | GYDVLTGYPDY |
| | | LSCAASGFTFSRFAMHWVR | (SEQ ID | (SEQ ID NO: 116) | (SEQ ID NO: 171) |
| | | QAPGKGLEWVAVISYQGRN | NO: 88) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 203) | | | |
| IPS:420849 | HV-14 | QVQLVESGGGVVQPGRSLR | RFAMH | VISYYGRNKYYAESVKG | GYDVLTGYPDY |
| | | LSCAASGFTFSRFAMHWVR | (SEQ ID | (SEQ ID NO: 117) | (SEQ ID NO: 171) |
| | | QAPGKGLEWVAVISYYGRN | NO: 88) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 204) | | | |
| IPS:420853 | HV-15 | QVQLVESGGGVVQPGRSLR | RFAMH | VISYNGNNKYYAESVKG | GYDVLTGYPDY |
| | | LSCAASGFTFSRFAMHWVR | (SEQ ID | (SEQ ID NO: 118) | (SEQ ID NO: 171) |
| | | QAPGKGLEWVAVISYNGNN | NO: 88) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 205) | | | |
| IPS:420857 | HV-16 | QVQLVESGGGVVQPGRSLR | RFAMH | VISYIGNNKYYAESVKG | GYDVLTGYPDY |
| | | LSCAASGFTFSRFAMHWVR | (SEQ ID | (SEQ ID NO: 119) | (SEQ ID NO: 171) |
| | | QAPGKGLEWVAVISYIGNN | NO: 88) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 206) | | | |
| IPS:420861 | HV-17 | QVQLVESGGGVVQPGRSLR | RFAMH | VISYQGNNKYYAESVKG | GYDVLTGYPDY |
| | | LSCAASGFTFSRFAMHWVR | (SEQ ID | (SEQ ID NO: 120) | (SEQ ID NO: 171) |
| | | QAPGKGLEWVAVISYQGNN | NO: 88) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 207) | | | |
| IPS:420865 | HV-18 | QVQLVESGGGVVQPGRSLR | RFAMH | VISYYGNNKYYAESVKG | GYDVLTGYPDY |
| | | LSCAASGFTFSRFAMHWVR | (SEQ ID | (SEQ ID NO: 121) | (SEQ ID NO: 171) |
| | | QAPGKGLEWVAVISYYGNN | NO: 88) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 208) | | | |
| iPS:420869 | HV-19 | QVQLVESGGGVVQPGRSLR | RFAMH | VISYNGGNKYYARSVKG | GYDVLTGYPDY |
| | | LSCAASGFTFSRFAMHWVR | (SEQ ID | (SEQ ID NO: 122) | (SEQ ID NO: 171) |
| | | QAPGKGLEWVAVISYNGGN | NO: 88) | | |
| | | KYYARSVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 209) | | | |
| iPS:420873 | HV-20 | QVQLVESGGGVVQPGRSLR | RFAMH | VISYIGGNKYYARSVKG | GYDVLTGYPDY |
| | | LSCAASGFTFSRFAMHWVR | (SEQ ID | (SEQ ID NO: 123) | (SEQ ID NO: 171) |
| | | QAPGKGLEWVAVISYIGGN | NO: 88) | | |
| | | KYYARSVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 210) | | | |
| iPS:420877 | HV-21 | QVQLVESGGGVVQPGRSLR | RFAMH | VISYQGGNKYYARSVKG | GYDVLTGYPDY |
| | | LSCAASGFTFSRFAMHWVR | (SEQ ID | (SEQ ID NO: 124) | (SEQ ID NO: 171) |
| | | QAPGKGLEWVAVISYQGGN | NO: 88) | | |
| | | KYYARSVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 211) | | | |
| iPS:420881 | HV-22 | QVQLVESGGGVVQPGRSLR | RFAMH | VISYYGGNKYYARSVKG | GYDVLTGYPDY |
| | | LSCAASGFTFSRFAMHWVR | (SEQ ID | (SEQ ID NO: 125) | (SEQ ID NO: 171) |
| | | QAPGKGLEWVAVISYYGGN | NO: 88) | | |
| | | KYYARSVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 212) | | | |
| iPS:420885 | HV-23 | QVQLVESGGGVVQPGRSLR | RFAMH | VISYNGGNKYYAESVKG | GYDILTGYPDY |
| | | LSCAASGFTFSRFAMHWVR | (SEQ ID | (SEQ ID NO: 107) | (SEQ ID NO: 172) |
| | | QAPGKGLEWVAVISYNGGN | NO: 88) | | |
| | | KYYAESVKGRFTISRDNSKN | | | |
| | | TLYLQMNSLRAEDTALFYC | | | |
| | | ARGYDILTGYPDYWGQGTL | | | |
| | | VTVSS (SEQ ID NO: 213) | | | |
| iPS:420889 | HV-24 | QVQLVESGGGVVQPGRSLR | RFAMH | VISYIGGNKYYAESVKG | GYDILTGYPDY |
| | | LSCAASGFTFSRFAMHWVR | (SEQ ID | (SEQ ID NO: 108) | (SEQ ID NO: 172) |
| | | QAPGKGLEWVAVISYIGGN | NO: 88) | | |
| | | KYYAESVKGRFTISRDNSKN | | | |
| | | TLYLQMNSLRAEDTALFYC | | | |
| | | ARGYDILTGYPDYWGQGTL | | | |
| | | VTVSS (SEQ ID NO: 214) | | | |
| iPS:420893 | HV-25 | QVQLVESGGGVVQPGRSLR | RFAMH | VISYQGGNKYYAESVKG | GYDILTGYPDY |
| | | LSCAASGFTFSRFAMHWVR | (SEQ ID | (SEQ ID NO: 109) | (SEQ ID NO: 172) |
| | | QAPGKGLEWVAVISYQGGN | NO: 88) | | |
| | | KYYAESVKGRFTISRDNSKN | | | |
| | | TLYLQMNSLRAEDTALFYC | | | |
| | | ARGYDILTGYPDYWGQGTL | | | |
| | | VTVSS (SEQ ID NO: 215) | | | |
| iPS:420897 | HV-26 | QVQLVESGGGVVQPGRSLR | RFAMH | VISYYGGNKYYAESVKG | GYDILTGYPDY |
| | | LSCAASGFTFSRFAMHWVR | (SEQ ID | (SEQ ID NO: 110) | (SEQ ID NO: 172) |
| | | QAPGKGLEWVAVISYYGGN | NO: 88) | | |
| | | KYYAESVKGRFTISRDNSKN | | | |
| | | TLYLQMNSLRAEDTALFYC | | | |
| | | ARGYDILTGYPDYWGQGTL | | | |
| | | VVTVSS (SEQ ID NO: 216) | | | |
| IPS:420901 | HV-32 | QVQLVESGGGVVQPGRSLR | RFAMH | VISYNGRNKYYARSVKG | GYDVLTGYPDY |
| | | LSCAASGFTFSRFAMHWVR | (SEQ ID | (SEQ ID NO: 128) | (SEQ ID NO: 171) |
| | | QAPGKGLEWVAVISYNGRN | NO: 88) | | |
| | | KYYARSVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 222) | | | |
| IPS:420908 | HV-33 | QVQLVESGGGVVQPGRSLR | RFAMH | VISYIGRNKYYARSVKG | GYDVLTGYPDY |
| | | LSCAASGFTFSRFAMHWVR | (SEQ ID | (SEQ ID NO: 129) | (SEQ ID NO: 171) |
| | | QAPGKGLEWVAVISYIGRNK | NO: 88) | | |
| | | YYARSVKGRFTISRDNSKNT | | | |
| | | LYLQMNSLRAEDTALFYCA | | | |
| | | RGYDVLTGYPDYWGQGTLV | | | |
| | | TVSS (SEQ ID NO: 223) | | | |
| IPS:420915 | HV-34 | QVQLVESGGGVVQPGRSLR | RFAMH | VISYQGRNKYYARSVKG | GYDVLTGYPDY |
| | | LSCAASGFTFSRFAMHWVR | (SEQ ID | (SEQ ID NO: 130) | (SEQ ID NO: 171) |
| | | QAPGKGLEWVAVISYQGRN | NO: 88) | | |
| | | KYYARSVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 224) | | | |
| IPS:420922 | HV-35 | QVQLVESGGGVVQPGRSLR | RFAMH | VISYYGRNKYYARSVKG | GYDVLTGYPDY |
| | | LSCAASGFTFSRFAMHWVR | (SEQ ID | (SEQ ID NO: 131) | (SEQ ID NO: 171) |
| | | QAPGKGLEWVAVISYYGRN | NO: 88) | | |
| | | KYYARSVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 225) | | | |
| IPS:420929 | HV-36 | QVQLVESGGGVVQPGRSLR | RFAMH | VISYNGNNTKYYARSVKG | GYDVLTGYPDY |
| | | LSCAASGFTFSRFAMHWVR | (SEQ ID | (SEQ ID NO: 132) | (SEQ ID NO: 171) |
| | | QAPGKGLEWVAVISYNGNN | NO: 88) | | |
| | | KYYARSVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 226) | | | |
| IPS:420936 | HV-37 | QVQLVESGGGVVQPGRSLR | RFAMH | VISYIGNNTKYYARSVKG | GYDVLTGYPDY |
| | | LSCAASGFTFSRFAMHWVR | (SEQ ID | (SEQ ID NO: 133) | (SEQ ID NO: 171) |
| | | QAPGKGLEWVAVISYIGNN | NO: 88) | | |
| | | KYYARSVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 227) | | | |
| IPS:420943 | HV-38 | QVQLVESGGGVVQPGRSLR | RFAMH | VISYQGNNTKYYARSVKG | GYDVLTGYPDY |
| | | LSCAASGFTFSRFAMHWVR | (SEQ ID | (SEQ ID NO: 134) | (SEQ ID NO: 171) |
| | | QAPGKGLEWVAVISYQGNN | NO: 88) | | |
| | | KYYARSVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 228) | | | |
| IPS:420950 | HV-39 | QVQLVESGGGVVQPGRSLR | RFAMH | VISYYGNNTKYYARSVKG | GYDVLTGYPDY |
| | | LSCAASGFTFSRFAMHWVR | (SEQ ID | (SEQ ID NO: 135) | (SEQ ID NO: 171) |
| | | QAPGKGLEWVAVISYYGNN | NO: 88) | | |
| | | KYYARSVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 229) | | | |
| iPS:420957 | HV-40 | QVQLVESGGGVVQPGRSLR | RFAMH | VISYNGRNKYYAESVKG | GYDILTGYPDY |
| | | LSCAASGFTFSRFAMHWVR | (SEQ ID | (SEQ ID NO: 114) | (SEQ ID NO: 172) |
| | | QAPGKGLEWVAVISYNGRN | NO: 88) | | |
| | | KYYAESVKGRFTISRDNSKN | | | |
| | | TLYLQMNSLRAEDTALFYC | | | |
| | | ARGYDILTGYPDYWGQGTL | | | |
| | | VTVSS (SEQ ID NO: 230) | | | |
| iPS:420964 | HV-41 | QVQLVESGGGVVQPGRSLR | RFAMH | VISYIGRNKYYAESVKG | GYDILTGYPDY |
| | | LSCAASGFTFSRFAMHWVR | (SEQ ID | (SEQ ID NO: 115) | (SEQ ID NO: 172) |
| | | QAPGKGLEWVAVISYIGRNK | NO: 88) | | |
| | | YYAESVKGRFTISRDNSKNT | | | |
| | | LYLQMNSLRAEDTALFYCA | | | |
| | | RGYDILTGYPDYWGQGTLV | | | |
| | | TVSS (SEQ ID NO: 231) | | | |
| iPS:420971 | HV-42 | QVQLVESGGGVVQPGRSLR | RFAMH | VISYQGRNKYYAESVKG | GYDILTGYPDY |
| | | LSCAASGFTFSRFAMHWVR | (SEQ ID | (SEQ ID NO: 116) | (SEQ ID NO: 172) |
| | | QAPGKGLEWVAVISYQGRN | NO: 88) | | |
| | | KYYAESVKGRFTISRDNSKN | | | |
| | | TLYLQMNSLRAEDTALFYC | | | |
| | | ARGYDILTGYPDYWGQGTL | | | |
| | | VTVSS (SEQ ID NO: 232) | | | |
| iPS:420978 | HV-43 | QVQLVESGGGVVQPGRSLR | RFAMH | VISYYGRNKYYAESVKG | GYDILTGYPDY |
| | | LSCAASGFTFSRFAMHWVR | (SEQ ID | (SEQ ID NO: 117) | (SEQ ID NO: 172) |
| | | QAPGKGLEWVAVISYYGRN | NO: 88) | | |
| | | KYYAESVKGRFTISRDNSKN | | | |
| | | TLYLQMNSLRAEDTALFYC | | | |
| | | ARGYDILTGYPDYWGQGTL | | | |
| | | VTVSS (SEQ ID NO: 233) | | | |
| iPS:420985 | HV-44 | QVQLVESGGGVVQPGRSLR | RFAMH | VISYNGNNKYYAESVKG | GYDILTGYPDY |
| | | LSCAASGFTFSRFAMHWVR | (SEQ ID | (SEQ ID NO: 118) | (SEQ ID NO: 172) |
| | | QAPGKGLEWVAVISYNGNN | NO: 88) | | |
| | | KYYAESVKGRFTISRDNSKN | | | |
| | | TLYLQMNSLRAEDTALFYC | | | |
| | | ARGYDILTGYPDYWGQGTL | | | |
| | | VTVSS (SEQ ID NO: 234) | | | |
| iPS:420992 | HV-45 | QVQLVESGGGVVQPGRSLR | RFAMH | VISYIGNNKYYAESVKG | GYDILTGYPDY |
| | | LSCAASGFTFSRFAMHWVR | (SEQ ID | (SEQ ID NO: 119) | (SEQ ID NO: 172) |
| | | QAPGKGLEWVAVISYIGNN | NO: 88) | | |
| | | KYYAESVKGRFTISRDNSKN | | | |
| | | TLYLQMNSLRAEDTALFYC | | | |
| | | ARGYDILTGYPDYWGQGTL | | | |
| | | VTVSS (SEQ ID NO: 235) | | | |
| iPS:420999 | HV-46 | QVQLVESGGGVVQPGRSLR | RFAMH | VISYQGNNKYYAESVKG | GYDILTGYPDY |
| | | LSCAASGFTFSRFAMHWVR | (SEQ ID | (SEQ ID NO: 120) | (SEQ ID NO: 172) |
| | | QAPGKGLEWVAVISYQGNN | NO: 88) | | |
| | | KYYAESVKGRFTISRDNSKN | | | |
| | | TLYLQMNSLRAEDTALFYC | | | |
| | | ARGYDILTGYPDYWGQGTL | | | |
| | | VTVSS (SEQ ID NO: 236) | | | |
| iPS:421006 | HV-47 | QVQLVESGGGVVQPGRSLR | RFAMH | VISYYGNNKYYAESVKG | GYDILTGYPDY |
| | | LSCAASGFTFSRFAMHWVR | (SEQ ID | (SEQ ID NO: 121) | (SEQ ID NO: 172) |
| | | QAPGKGLEWVAVISYYGNN | NO: 88) | | |
| | | KYYAESVKGRFTISRDNSKN | | | |
| | | TLYLQMNSLRAEDTALFYC | | | |
| | | ARGYDILTGYPDYWGQGTL | | | |
| | | VTVSS (SEQ ID NO: 237) | | | |
| iPS:421013 | HV-48 | QVQLVESGGGVVQPGRSLR | RFAMH | VISYDGRNKYYARSVKG | GYDILTGYPDY |
| | | LSCAASGFTFSRFAMHWVR | (SEQ ID | (SEQ ID NO: 126) | (SEQ ID NO: 172) |
| | | QAPGKGLEWVAVISYDGRN | NO: 88) | | |
| | | KYYARSVKGRFTISRDNSKN | | | |
| | | TLYLQMNSLRAEDTALFYC | | | |
| | | ARGYDILTGYPDYWGQGTL | | | |
| | | VTVSS (SEQ ID NO: 238) | | | |
| iPS:421020 | HV-49 | QVQLVESGGGVVQPGRSLR | RFAMH | VISYDGNNKYYARSVKG | GYDILTGYPDY |
| | | LSCAASGFTFSRFAMHWVR | (SEQ ID | (SEQ ID NO: 127) | (SEQ ID NO: 172) |
| | | QAPGKGLEWVAVISYDGNN | NO: 88) | | |
| | | KYYARSVKGRFTISRDNSKN | | | |
| | | TLYLQMNSLRAEDTALFYC | | | |
| | | ARGYDILTGYPDYWGQGTL | | | |
| | | VTVSS (SEQ ID NO: 239) | | | |
| iPS:421027 | HV-32 | QVQLVESGGGVVQPGRSLR | RFAMH | VISYNGRNKYYARSVKG | GYDVLTGYPDY |
| | | LSCAASGFTFSRFAMHWVR | (SEQ ID | (SEQ ID NO: 128) | (SEQ ID NO: 171) |
| | | QAPGKGLEWVAVISYNGRN | NO: 88) | | |
| | | KYYARSVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 222) | | | |
| IPS:421031 | HV-33 | QVQLVESGGGVVQPGRSLR | RFAMH | VISYIGRNKYYARSVKG | GYDVLTGYPDY |
| | | LSCAASGFTFSRFAMHWVR | (SEQ ID | (SEQ ID NO: 129) | (SEQ ID NO: 171) |
| | | QAPGKGLEWVAVISYIGRNK | NO: 88) | | |
| | | YYARSVKGRFTISRDNSKNT | | | |
| | | LYLQMNSLRAEDTALFYCA | | | |
| | | RGYDVLTGYPDYWGQGTLV | | | |
| | | TVSS (SEQ ID NO: 223) | | | |
| IPS:421035 | HV-34 | QVQLVESGGGVVQPGRSLR | RFAMH | VISYQGRNKYYARSVKG | GYDVLTGYPDY |
| | | LSCAASGFTFSRFAMHWVR | (SEQ ID | (SEQ ID NO: 130) | (SEQ ID NO: 171) |
| | | QAPGKGLEWVAVISYQGRN | NO: 88) | | |
| | | KYYARSVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 224) | | | |
| IPS:421039 | HV-35 | QVQLVESGGGVVQPGRSLR | RFAMH | VISYYGRNKYYARSVKG | GYDVLTGYPDY |
| | | LSCAASGFTFSRFAMHWVR | (SEQ ID | (SEQ ID NO: 131) | (SEQ ID NO: 171) |
| | | QAPGKGLEWVAVISYYGRN | NO: 88) | | |
| | | KYYARSVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 225) | | | |
| iPS:421043 | HV-36 | QVQLVESGGGVVQPGRSLR | RFAMH | VISYNGNNKYYARSVKG | GYDVLTGYPDY |
| | | LSCAASGFTFSRFAMHWVR | (SEQ ID | (SEQ ID NO: 132) | (SEQ ID NO: 171) |
| | | QAPGKGLEWVAVISYNGNN | NO: 88) | | |
| | | KYYARSVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 226) | | | |
| iPS:421047 | HV-37 | QVQLVESGGGVVQPGRSLR | RFAMH | VISYIGNNKYYARSVKG | GYDVLTGYPDY |
| | | LSCAASGFTFSRFAMHWVR | (SEQ ID | (SEQ ID NO: 133) | (SEQ ID NO: 171) |
| | | QAPGKGLEWVAVISYIGNN | NO: 88) | | |
| | | KYYARSVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 227) | | | |
| IPS:421051 | HV-38 | QVQLVESGGGVVQPGRSLR | RFAMH | VISYQGNNKYYARSVKG | GYDVLTGYPDY |
| | | LSCAASGFTFSRFAMHWVR | (SEQ ID | (SEQ ID NO: 134) | (SEQ ID NO: 171) |
| | | QAPGKGLEWVAVISYQGNN | NO: 88) | | |
| | | KYYARSVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 228) | | | |
| IPS:421055 | HV-39 | QVQLVESGGGVVQPGRSLR | RFAMH | VISYYGNNKYYARSVKG | GYDVLTGYPDY |
| | | LSCAASGFTFSRFAMHWVR | (SEQ ID | (SEQ ID NO: 135) | (SEQ ID NO: 171) |
| | | QAPGKGLEWVAVISYYGNN | NO: 88) | | |
| | | KYYARSVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 229) | | | |
| iPS:421059 | HV-40 | QVQLVESGGGVVQPGRSLR | RFAMH | VISYNGRNKYYAESVKG | GYDILTGYPDY |
| | | LSCAASGFTFSRFAMHWVR | (SEQ ID | (SEQ ID NO: 114) | (SEQ ID NO: 172) |
| | | QAPGKGLEWVAVISYNGRN | NO: 88) | | |
| | | KYYAESVKGRFTISRDNSKN | | | |
| | | TLYLQMNSLRAEDTALFYC | | | |
| | | ARGYDILTGYPDYWGQGTL | | | |
| | | VTVSS (SEQ ID NO: 230) | | | |
| iPS:421063 | HV-41 | QVQLVESGGGVVQPGRSLR | RFAMH | VISYIGRNKYYAESVKG | GYDILTGYPDY |
| | | LSCAASGFTFSRFAMHWVR | (SEQ ID | (SEQ ID NO: 115) | (SEQ ID NO: 172) |
| | | QAPGKGLEWVAVISYIGRNK | NO: 88) | | |
| | | YYAESVKGRFTISRDNSKNT | | | |
| | | LYLQMNSLRAEDTALFYCA | | | |
| | | RGYDILTGYPDYWGQGTLV | | | |
| | | TVSS (SEQ ID NO: 231) | | | |
| iPS:421067 | HV-42 | QVQLVESGGGVVQPGRSLR | RFAMH | VISYQGRNKYYAESVKG | GYDILTGYPDY |
| | | LSCAASGFTFSRFAMHWVR | (SEQ ID | (SEQ ID NO: 116) | (SEQ ID NO: 172) |
| | | QAPGKGLEWVAVISYQGRN | NO: 88) | | |
| | | KYYAESVKGRFTISRDNSKN | | | |
| | | TLYLQMNSLRAEDTALFYC | | | |
| | | ARGYDILTGYPDYWGQGTL | | | |
| | | VTVSS (SEQ ID NO: 232) | | | |
| iPS:421071 | HV-43 | QVQLVESGGGVVQPGRSLR | RFAMH | VISYYGRNKYYAESVKG | GYDILTGYPDY |
| | | LSCAASGFTFSRFAMHWVR | (SEQ ID | (SEQ ID NO: 117) | (SEQ ID NO: 172) |
| | | QAPGKGLEWVAVISYYGRN | NO: 88) | | |
| | | KYYAESVKGRFTISRDNSKN | | | |
| | | TLYLQMNSLRAEDTALFYC | | | |
| | | ARGYDILTGYPDYWGQGTL | | | |
| | | VTVSS (SEQ ID NO: 233) | | | |
| iPS:421075 | HV-44 | QVQLVESGGGVVQPGRSLR | RFAMH | VISYNGNNKYYAESVKG | GYDILTGYPDY |
| | | LSCAASGFTFSRFAMHWVR | (SEQ ID | (SEQ ID NO: 118) | (SEQ ID NO: 172) |
| | | QAPGKGLEWVAVISYNGNN | NO: 88) | | |
| | | KYYAESVKGRFTISRDNSKN | | | |
| | | TLYLQMNSLRAEDTALFYC | | | |
| | | ARGYDILTGYPDYWGQGTL | | | |
| | | VTVSS (SEQ ID NO: 234) | | | |
| iPS:421079 | HV-45 | QVQLVESGGGVVQPGRSLR | RFAMH | VISYIGNNKYYAESVKG | GYDILTGYPDY |
| | | LSCAASGFTFSRFAMHWVR | (SEQ ID | (SEQ ID NO: 119) | (SEQ ID NO: 172) |
| | | QAPGKGLEWVAVISYIGNN | NO: 88) | | |
| | | KYYAESVKGRFTISRDNSKN | | | |
| | | TLYLQMNSLRAEDTALFYC | | | |
| | | ARGYDILTGYPDYWGQGTL | | | |
| | | VTVSS (SEQ ID NO: 235) | | | |
| iPS:421083 | HV-46 | QVQLVESGGGVVQPGRSLR | RFAMH | VISYQGNNKYYAESVKG | GYDILTGYPDY |
| | | LSCAASGFTFSRFAMHWVR | (SEQ ID | (SEQ ID NO: 120) | (SEQ ID NO: 172) |
| | | QAPGKGLEWVAVISYQGNN | NO: 88) | | |
| | | KYYAESVKGRFTISRDNSKN | | | |
| | | TLYLQMNSLRAEDTALFYC | | | |
| | | ARGYDILTGYPDYWGQGTL | | | |
| | | VTVSS (SEQ ID NO: 236) | | | |
| iPS:421087 | HV-47 | QVQLVESGGGVVQPGRSLR | RFAMH | VISYYGNNKYYAESVKG | GYDILTGYPDY |
| | | LSCAASGFTFSRFAMHWVR | (SEQ ID | (SEQ ID NO: 121) | (SEQ ID NO: 172) |
| | | QAPGKGLEWVAVISYYGNN | NO: 88) | | |
| | | KYYAESVKGRFTISRDNSKN | | | |
| | | TLYLQMNSLRAEDTALFYC | | | |
| | | ARGYDILTGYPDYWGQGTL | | | |
| | | VTVSS (SEQ ID NO: 237) | | | |
| iPS:421091 | HV-50 | QVQLVESGGGVVQPGRSLR | RFAMH | VISYNGRNKYYARSVKG | GYDILTGYPDY |
| | | LSCAASGFTFSRFAMHWVR | (SEQ ID | (SEQ ID NO: 128) | (SEQ ID NO: 172) |
| | | QAPGKGLEWVAVISYNGRN | NO: 88) | | |
| | | KYYARSVKGRFTISRDNSKN | | | |
| | | TLYLQMNSLRAEDTALFYC | | | |
| | | ARGYDILTGYPDYWGQGTL | | | |
| | | VTVSS (SEQ ID NO: 240) | | | |
| iPS:421098 | HV-51 | QVQLVESGGGVVQPGRSLR | RFAMH | VISYIGRNKYYARSVKG | GYDILTGYPDY |
| | | LSCAASGFTFSRFAMHWVR | (SEQ ID | (SEQ ID NO: 129) | (SEQ ID NO: 172) |
| | | QAPGKGLEWVAVISYIGRNK | NO: 88) | | |
| | | YYARSVKGRFTISRDNSKNT | | | |
| | | LYLQMNSLRAEDTALFYCA | | | |
| | | RGYDILTGYPDYWGQGTLV | | | |
| | | TVSS (SEQ ID NO: 241) | | | |
| iPS:421105 | HV-52 | QVQLVESGGGVVQPGRSLR | RFAMH | VISYQGRNKYYARSVKG | GYDILTGYPDY |
| | | LSCAASGFTFSRFAMHWVR | (SEQ ID | (SEQ ID NO: 130) | (SEQ ID NO: 172) |
| | | QAPGKGLEWVAVISYQGRN | NO: 88) | | |
| | | KYYARSVKGRFTISRDNSKN | | | |
| | | TLYLQMNSLRAEDTALFYC | | | |
| | | ARGYDILTGYPDYWGQGTL | | | |
| | | VTVSS (SEQ ID NO: 242) | | | |
| iPS:421112 | HV-53 | QVQLVESGGGVVQPGRSLR | RFAMH | VISYYGRNKYYARSVKG | GYDILTGYPDY |
| | | LSCAASGFTFSRFAMHWVR | (SEQ ID | (SEQ ID NO: 131) | (SEQ ID NO: 172) |
| | | QAPGKGLEWVAVISYYGRN | NO: 88) | | |
| | | KYYARSVKGRFTISRDNSKN | | | |
| | | TLYLQMNSLRAEDTALFYC | | | |
| | | ARGYDILTGYPDYWGQGTL | | | |
| | | VTVSS (SEQ ID NO: 243) | | | |
| iPS:421119 | HV-54 | QVQLVESGGGVVQPGRSLR | RFAMH | VISYNGNNTKYYARSVKG | GYDILTGYPDY |
| | | LSCAASGFTFSRFAMHWVR | (SEQ ID | (SEQ ID NO: 132) | (SEQ ID NO: 172) |
| | | QAPGKGLEWVAVISYNGNN | NO: 88) | | |
| | | KYYARSVKGRFTISRDNSKN | | | |
| | | TLYLQMNSLRAEDTALFYC | | | |
| | | ARGYDILTGYPDYWGQGTL | | | |
| | | VTVSS (SEQ ID NO: 244) | | | |
| iPS:421126 | HV-55 | QVQLVESGGGVVQPGRSLR | RFAMH | VISYIGNNTKYYARSVKG | GYDILTGYPDY |
| | | LSCAASGFTFSRFAMHWVR | (SEQ ID | (SEQ ID NO: 133) | (SEQ ID NO: 172) |
| | | QAPGKGLEWVAVISYIGNN | NO: 88) | | |
| | | KYYARSVKGRFTISRDNSKN | | | |
| | | TLYLQMNSLRAEDTALFYC | | | |
| | | ARGYDILTGYPDYWGQGTL | | | |
| | | VTVSS (SEQ ID NO: 245) | | | |
| iPS:421133 | HV-56 | QVQLVESGGGVVQPGRSLR | RFAMH | VISYQGNNTKYYARSVKG | GYDILTGYPDY |
| | | LSCAASGFTFSRFAMHWVR | (SEQ ID | (SEQ ID NO: 134) | (SEQ ID NO: 172) |
| | | QAPGKGLEWVAVISYQGNN | NO: 88) | | |
| | | KYYARSVKGRFTISRDNSKN | | | |
| | | TLYLQMNSLRAEDTALFYC | | | |
| | | ARGYDILTGYPDYWGQGTL | | | |
| | | VTVSS (SEQ ID NO: 246) | | | |
| iPS:421140 | HV-57 | QVQLVESGGGVVQPGRSLR | RFAMH | VISYYGNNTKYYARSVKG | GYDILTGYPDY |
| | | LSCAASGFTFSRFAMHWVR | (SEQ ID | (SEQ ID NO: 135) | (SEQ ID NO: 172) |
| | | QAPGKGLEWVAVISYYGNN | NO: 88) | | |
| | | KYYARSVKGRFTISRDNSKN | | | |
| | | TLYLQMNSLRAEDTALFYC | | | |
| | | ARGYDILTGYPDYWGQGTL | | | |
| | | VTVSS (SEQ ID NO: 247) | | | |
| iPS:421147 | HV-57 | QVQLVESGGGVVQPGRSLR | RFAMH | VISYYGNNTKYYARSVKG | GYDILTGYPDY |
| | | LSCAASGFTFSRFAMHWVR | (SEQ ID | (SEQ ID NO: 135) | (SEQ ID NO: 172) |
| | | QAPGKGLEWVAVISYYGNN | NO: 88) | | |
| | | KYYARSVKGRFTISRDNSKN | | | |
| | | TLYLQMNSLRAEDTALFYC | | | |
| | | ARGYDILTGYPDYWGQGTL | | | |
| | | VTVSS (SEQ ID NO: 247) | | | |
| iPS:421151 | HV-01 | QVQLVESGGGVVQPGRSLR | RFAMH | VISYDGGNKYYAESVKG | GYDVLTGYPDY |
| | | LSCAASGFTFSRFAMHWVR | (SEQ ID | (SEQ ID NO: 106) | (SEQ ID NO: 171) |
| | | QAPGKGLEWVAVISYDGGN | NO: 88) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 191) | | | |
| iPS:391478 | HV-01 | QVQLVESGGGVVQPGRSLR | RFAMH | VISYDGGNKYYAESVKG | GYDVLTGYPDY |
| | | LSCAASGFTFSRFAMHWVR | (SEQ ID | (SEQ ID NO: 106) | (SEQ ID NO: 171) |
| | | QAPGKGLEWVAVISYDGGN | NO: 88) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 191) | | | |
| iPS:421157 | HV-01 | QVQLVESGGGVVQPGRSLR | RFAMH | VISYDGGNKYYAESVKG | GYDVLTGYPDY |
| | | LSCAASGFTFSRFAMHWVR | (SEQ ID | (SEQ ID NO: 106) | (SEQ ID NO: 171) |
| | | QAPGKGLEWVAVISYDGGN | NO: 88) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 191) | | | |
| iPS:421163 | HV-58 | QVQLVESGGGVVQPGRSLR | HFAMH | VISYDGGNKYYAESVKG | GYDVLTGYPDY |
| | | LSCAASGFTFSHFAMHWVR | (SEQ ID | (SEQ ID NO: 106) | (SEQ ID NO: 171) |
| | | QAPGKGLEWVAVISYDGGN | NO: 89) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 248) | | | |
| iPS:391578 | HV-59 | QVQLVESGGGVVQPGRSLR | RYAMH | VISYDGGNKYYAESVKG | GYDVLTGYPDY |
| | | LSCAASGFTFSRYAMHWVR | (SEQ ID | (SEQ ID NO: 106) | (SEQ ID NO: 171) |
| | | QAPGKGLEWVAVISYDGGN | NO: 90) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 249) | | | |
| iPS:421170 | HV-60 | QVQLVESGGGVVQPGRSLR | RFAMH | VISFDGGNKYYAESVKG | GYDVLTGYPDY |
| | | LSCAASGFTFSRFAMHWVR | (SEQ ID | (SEQ ID NO: 136) | (SEQ ID NO: 171) |
| | | QAPGKGLEWVAVISFDGGN | NO: 88) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 250) | | | |
| iPS:421176 | HV-61 | QVQLVESGGGVVQPGRSLR | RFAMH | VISYDGANKYYAESVKG | GYDVLTGYPDY |
| | | LSCAASGFTFSRFAMHWVR | (SEQ ID | (SEQ ID NO: 137) | (SEQ ID NO: 171) |
| | | QAPGKGLEWVAVISYDGAN | NO: 88) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 251) | | | |
| iPS:421182 | HV-62 | QVQLVESGGGVVQPGRSLR | RFAMH | VISYDGGNKYYAESVKG | GYDFLTGYPDY |
| | | LSCAASGFTFSRFAMHWVR | (SEQ ID | (SEQ ID NO: 106) | (SEQ ID NO: 173) |
| | | QAPGKGLEWVAVISYDGGN | NO: 88) | | |
| | | KYYAESVKGRFTISRDNSKN | | | |
| | | TLYLQMNSLRAEDTALFYC | | | |
| | | ARGYDFLTGYPDYWGQGTL | | | |
| | | VTVSS (SEQ ID NO: 252) | | | |
| iPS:421189 | HV-01 | QVQLVESGGGVVQPGRSLR | RFAMH | VISYDGGNKYYAESVKG | GYDVLTGYPDY |
| | | LSCAASGFTFSRFAMHWVR | (SEQ ID | (SEQ ID NO: 106) | (SEQ ID NO: 171) |
| | | QAPGKGLEWVAVISYDGGN | NO: 88) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 191) | | | |
| iPS:421195 | HV-01 | QVQLVESGGGVVQPGRSLR | RFAMH | VISYDGGNKYYAESVKG | GYDVLTGYPDY |
| | | LSCAASGFTFSRFAMHWVR | (SEQ ID | (SEQ ID NO: 106) | (SEQ ID NO: 171) |
| | | QAPGKGLEWVAVISYDGGN | NO: 88) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 191) | | | |
| iPS:421201 | HV-01 | QVQLVESGGGVVQPGRSLR | RFAMH | VISYDGGNKYYAESVKG | GYDVLTGYPDY |
| | | LSCAASGFTFSRFAMHWVR | (SEQ ID | (SEQ ID NO: 106) | (SEQ ID NO: 171) |
| | | QAPGKGLEWVAVISYDGGN | NO: 88) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 191) | | | |
| iPS:421207 | HV-58 | QVQLVESGGGVVQPGRSLR | HFAMH | VISYDGGNKYYAESVKG | GYDVLTGYPDY |
| | | LSCAASGFTFSHFAMHWVR | (SEQ ID | (SEQ ID NO: 106) | (SEQ ID NO: 171) |
| | | QAPGKGLEWVAVISYDGGN | NO: 89) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 248) | | | |
| iPS:421211 | HV-59 | QVQLVESGGGVVQPGRSLR | RYAMH | VISYDGGNKYYAESVKG | GYDVLTGYPDY |
| | | LSCAASGFTFSRYAMHWVR | (SEQ ID | (SEQ ID NO: 106) | (SEQ ID NO: 171) |
| | | QAPGKGLEWVAVISYDGGN | NO: 90) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 249) | | | |
| iPS:421215 | HV-60 | QVQLVESGGGVVQPGRSLR | RFAMH | VISFDGGNKYYAESVKG | GYDVLTGYPDY |
| | | LSCAASGFTFSRFAMHWVR | (SEQ ID | (SEQ ID NO: 136) | (SEQ ID NO: 171) |
| | | QAPGKGLEWVAVISFDGGN | NO: 88) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 250) | | | |
| iPS:421219 | HV-61 | QVQLVESGGGVVQPGRSLR | RFAMH | VISYDGANKYYAESVKG | GYDVLTGYPDY |
| | | LSCAASGFTFSRFAMHWVR | (SEQ ID | (SEQ ID NO: 137) | (SEQ ID NO: 171) |
| | | QAPGKGLEWVAVISYDGAN | NO: 88) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 251) | | | |
| iPS:421223 | HV-62 | QVQLVESGGGVVQPGRSLR | RFAMH | VISYDGGNKYYAESVKG | GYDFLTGYPDY |
| | | LSCAASGFTFSRFAMHWVR | (SEQ ID | (SEQ ID NO: 106) | (SEQ ID NO: 173) |
| | | QAPGKGLEWVAVISYDGGN | NO: 88) | | |
| | | KYYAESVKGRFTISRDNSKN | | | |
| | | TLYLQMNSLRAEDTALFYC | | | |
| | | ARGYDFLTGYPDYWGQGTL | | | |
| | | VTVSS (SEQ ID NO: 252) | | | |
| iPS:421227 | HV-03 | QVQLVESGGGVVQPGRSLR | RFAMH | VISYNGGNKYYAESVKG | GYDVLTGYPDY |
| | | LSCAASGFTFSRFAMHWVR | (SEQ ID | (SEQ ID NO: 107) | (SEQ ID NO: 171) |
| | | QAPGKGLEWVAVISYNGGN | NO: 88) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 193) | | | |
| iPS:421231 | HV-03 | QVQLVESGGGVVQPGRSLR | RFAMH | VISYNGGNKYYAESVKG | GYDVLTGYPDY |
| | | LSCAASGFTFSRFAMHWVR | (SEQ ID | (SEQ ID NO: 107) | (SEQ ID NO: 171) |
| | | QAPGKGLEWVAVISYNGGN | NO: 88) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 193) | | | |
| iPS:421235 | HV-03 | QVQLVESGGGVVQPGRSLR | RFAMH | VISYNGGNKYYAESVKG | GYDVLTGYPDY |
| | | LSCAASGFTFSRFAMHWVR | (SEQ ID | (SEQ ID NO: 107) | (SEQ ID NO: 171) |
| | | QAPGKGLEWVAVISYNGGN | NO: 88) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 193) | | | |
| iPS:421239 | HV-63 | QVQLVESGGGVVQPGRSLR | HFAMH | VISYNGGNKYYAESVKG | GYDVLTGYPDY |
| | | LSCAASGFTFSHFAMHWVR | (SEQ ID | (SEQ ID NO: 107) | (SEQ ID NO: 171) |
| | | QAPGKGLEWVAVISYNGGN | NO: 89) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 253) | | | |
| iPS:421246 | HV-64 | QVQLVESGGGVVQPGRSLR | RYAMH | VISYNGGNKYYAESVKG | GYDVLTGYPDY |
| | | LSCAASGFTFSRYAMHWVR | (SEQ ID | (SEQ ID NO: 107) | (SEQ ID NO: 171) |
| | | QAPGKGLEWVAVISYNGGN | NO: 90) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 254) | | | |
| iPS:421253 | HV-65 | QVQLVESGGGVVQPGRSLR | RFAMH | VISFNGGNKYYAESVKG | GYDVLTGYPDY |
| | | LSCAASGFTFSRFAMHWVR | (SEQ ID | (SEQ ID NO: 138) | (SEQ ID NO: 171) |
| | | QAPGKGLEWVAVISFNGGN | NO: 88) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 255) | | | |
| iPS:421260 | HV-66 | QVQLVESGGGVVQPGRSLR | RFAMH | VISYNGANKYYAESVKG | GYDVLTGYPDY |
| | | LSCAASGFTFSRFAMHWVR | (SEQ ID | (SEQ ID NO: 139) | (SEQ ID NO: 171) |
| | | QAPGKGLEWVAVISYNGAN | NO: 88) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 256) | | | |
| iPS:421267 | HV-67 | QVQLVESGGGVVQPGRSLR | RFAMH | VISYNGGNKYYAESVKG | GYDFLTGYPDY |
| | | LSCAASGFTFSRFAMHWVR | (SEQ ID | (SEQ ID NO: 107) | (SEQ ID NO: 173) |
| | | QAPGKGLEWVAVISYNGGN | NO: 88) | | |
| | | KYYAESVKGRFTISRDNSKN | | | |
| | | TLYLQMNSLRAEDTALFYC | | | |
| | | ARGYDFLTGYPDYWGQGTL | | | |
| | | VTVSS (SEQ ID NO: 257) | | | |
| iPS:421274 | HV-07 | QVQLVESGGGVVQPGRSLR | RFAMH | VISYDGRNKYYAESVKG | GYDVLTGYPDY |
| | | LSCAASGFTFSRFAMHWVR | (SEQ ID | (SEQ ID NO: 111) | (SEQ ID NO: 171) |
| | | QAPGKGLEWVAVISYDGRN | NO: 88) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 197) | | | |
| iPS:421278 | HV-07 | QVQLVESGGGVVQPGRSLR | RFAMH | VISYDGRNKYYAESVKG | GYDVLTGYPDY |
| | | LSCAASGFTFSRFAMHWVR | (SEQ ID | (SEQ ID NO: 111) | (SEQ ID NO: 171) |
| | | QAPGKGLEWVAVISYDGRN | NO: 88) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 197) | | | |
| iPS:421282 | HV-07 | QVQLVESGGGVVQPGRSLR | RFAMH | VISYDGRNKYYAESVKG | GYDVLTGYPDY |
| | | LSCAASGFTFSRFAMHWVR | (SEQ ID | (SEQ ID NO: 111) | (SEQ ID NO: 171) |
| | | QAPGKGLEWVAVISYDGRN | NO: 88) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 197) | | | |
| iPS:421286 | HV-68 | QVQLVESGGGVVQPGRSLR | HFAMH | VISYDGRNKYYAESVKG | GYDVLTGYPDY |
| | | LSCAASGFTFSHFAMHWVR | (SEQ ID | (SEQ ID NO: 111) | (SEQ ID NO: 171) |
| | | QAPGKGLEWVAVISYDGRN | NO: 89) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 258) | | | |
| iPS:421293 | HV-69 | QVQLVESGGGVVQPGRSLR | RYAMH | VISYDGRNKYYAESVKG | GYDVLTGYPDY |
| | | LSCAASGFTFSRYAMHWVR | (SEQ ID | (SEQ ID NO: 111) | (SEQ ID NO: 171) |
| | | QAPGKGLEWVAVISYDGRN | NO: 90) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 259) | | | |
| iPS:421300 | HV-70 | QVQLVESGGGVVQPGRSLR | RFAMH | VISFDGRNKYYAESVKG | GYDVLTGYPDY |
| | | LSCAASGFTFSRFAMHWVR | (SEQ ID | (SEQ ID NO: 140) | (SEQ ID NO: 171) |
| | | QAPGKGLEWVAVISFDGRN | NO: 88) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 260) | | | |
| iPS:421307 | HV-71 | QVQLVESGGGVVQPGRSLR | RFAMH | VISYDGRNKYYAESVKG | GYDFLTGYPDY |
| | | LSCAASGFTFSRFAMHWVR | (SEQ ID | (SEQ ID NO: 111) | (SEQ ID NO: 173) |
| | | QAPGKGLEWVAVISYDGRN | NO: 88) | | |
| | | KYYAESVKGRFTISRDNSKN | | | |
| | | TLYLQMNSLRAEDTALFYC | | | |
| | | ARGYDFLTGYPDYWGQGTL | | | |
| | | VTVSS (SEQ ID NO: 261) | | | |
| iPS:421314 | HV-09 | QVQLVESGGGVVQPGRSLR | RFAMH | VISYDGGNKYYARSVKG | GYDVLTGYPDY |
| | | LSCAASGFTFSRFAMHWVR | (SEQ ID | (SEQ ID NO: 113) | (SEQ ID NO: 171) |
| | | QAPGKGLEWVAVISYDGGN | NO: 88) | | |
| | | KYYARSVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 199) | | | |
| iPS:421318 | HV-09 | QVQLVESGGGVVQPGRSLR | RFAMH | VISYDGGNKYYARSVKG | GYDVLTGYPDY |
| | | LSCAASGFTFSRFAMHWVR | (SEQ ID | (SEQ ID NO: 113) | (SEQ ID NO: 171) |
| | | QAPGKGLEWVAVISYDGGN | NO: 88) | | |
| | | KYYARSVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 199) | | | |
| iPS:421322 | HV-09 | QVQLVESGGGVVQPGRSLR | RFAMH | VISYDGGNKYYARSVKG | GYDVLTGYPDY |
| | | LSCAASGFTFSRFAMHWVR | (SEQ ID | (SEQ ID NO: 113) | (SEQ ID NO: 171) |
| | | QAPGKGLEWVAVISYDGGN | NO: 88) | | |
| | | KYYARSVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 199) | | | |
| iPS:421326 | HV-72 | QVQLVESGGGVVQPGRSLR | HFAMH | VISYDGGNKYYARSVKG | GYDVLTGYPDY |
| | | LSCAASGFTFSHFAMHWVR | (SEQ ID | (SEQ ID NO: 113) | (SEQ ID NO: 171) |
| | | QAPGKGLEWVAVISYDGGN | NO: 89) | | |
| | | KYYARSVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 262) | | | |
| iPS:421333 | HV-73 | QVQLVESGGGVVQPGRSLR | RYAMH | VISYDGGNKYYARSVKG | GYDVLTGYPDY |
| | | LSCAASGFTFSRYAMHWVR | (SEQ ID | (SEQ ID NO: 113) | (SEQ ID NO: 171) |
| | | QAPGKGLEWVAVISYDGGN | NO: 90) | | |
| | | KYYARSVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 263) | | | |
| iPS:421340 | HV-74 | QVQLVESGGGVVQPGRSLR | RFAMH | VISFDGGNKYYARSVKG | GYDVLTGYPDY |
| | | LSCAASGFTFSRFAMHWVR | (SEQ ID | (SEQ ID NO: 141) | (SEQ ID NO: 171) |
| | | QAPGKGLEWVAVISFDGGN | NO: 88) | | |
| | | KYYARSVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 264) | | | |
| iPS:421347 | HV-75 | QVQLVESGGGVVQPGRSLR | RFAMH | VISYDGANKYYARSVKG | GYDVLTGYPDY |
| | | LSCAASGFTFSRFAMHWVR | (SEQ ID | (SEQ ID NO: 142) | (SEQ ID NO: 171) |
| | | QAPGKGLEWVAVISYDGAN | NO: 88) | | |
| | | KYYARSVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 265) | | | |
| iPS:421354 | HV-76 | QVQLVESGGGVVQPGRSLR | RFAMH | VISYDGGNKYYARSVKG | GYDFLTGYPDY |
| | | LSCAASGFTFSRFAMHWVR | (SEQ ID | (SEQ ID NO: 113) | (SEQ ID NO: 173) |
| | | QAPGKGLEWVAVISYDGGN | NO: 88) | | |
| | | KYYARSVKGRFTISRDNSKN | | | |
| | | TLYLQMNSLRAEDTALFYC | | | |
| | | ARGYDFLTGYPDYWGQGTL | | | |
| | | VTVSS (SEQ ID NO: 266) | | | |
| iPS:421361 | HV-10 | QVQLVESGGGVVQPGRSLR | RFAMH | VISYDGGNKYYAESVKG | GYDILTGYPDY |
| | | LSCAASGFTFSRFAMHWVR | (SEQ ID | (SEQ ID NO: 106) | (SEQ ID NO: 172) |
| | | QAPGKGLEWVAVISYDGGN | NO: 88) | | |
| | | KYYAESVKGRFTISRDNSKN | | | |
| | | TLYLQMNSLRAEDTALFYC | | | |
| | | ARGYDILTGYPDYWGQGTL | | | |
| | | VTVSS (SEQ ID NO: 200) | | | |
| iPS:421365 | HV-10 | QVQLVESGGGVVQPGRSLR | RFAMH | VISYDGGNKYYAESVKG | GYDILTGYPDY |
| | | LSCAASGFTFSRFAMHWVR | (SEQ ID | (SEQ ID NO: 106) | (SEQ ID NO: 172) |
| | | QAPGKGLEWVAVISYDGGN | NO: 88) | | |
| | | KYYAESVKGRFTISRDNSKN | | | |
| | | TLYLQMNSLRAEDTALFYC | | | |
| | | ARGYDILTGYPDYWGQGTL | | | |
| | | VTVSS (SEQ ID NO: 200) | | | |
| iPS:421369 | HV-10 | QVQLVESGGGVVQPGRSLR | RFAMH | VISYDGGNKYYAESVKG | GYDILTGYPDY |
| | | LSCAASGFTFSRFAMHWVR | (SEQ ID | (SEQ ID NO: 106) | (SEQ ID NO: 172) |
| | | QAPGKGLEWVAVISYDGGN | NO: 88) | | |
| | | KYYAESVKGRFTISRDNSKN | | | |
| | | TLYLQMNSLRAEDTALFYC | | | |
| | | ARGYDILTGYPDYWGQGTL | | | |
| | | VTVSS (SEQ ID NO: 200) | | | |
| iPS:421373 | HV-77 | QVQLVESGGGVVQPGRSLR | HFAMH | VISYDGGNKYYAESVKG | GYDILTGYPDY |
| | | LSCAASGFTFSHFAMHWVR | (SEQ ID | (SEQ ID NO: 106) | (SEQ ID NO: 172) |
| | | QAPGKGLEWVAVISYDGGN | NO: 89) | | |
| | | KYYAESVKGRFTISRDNSKN | | | |
| | | TLYLQMNSLRAEDTALFYC | | | |
| | | ARGYDILTGYPDYWGQGTL | | | |
| | | VTVSS (SEQ ID NO: 267) | | | |
| iPS:421380 | HV-78 | QVQLVESGGGVVQPGRSLR | RYAMH | VISYDGGNKYYAESVKG | GYDILTGYPDY |
| | | LSCAASGFTFSRYAMHWVR | (SEQ ID | (SEQ ID NO: 106) | (SEQ ID NO: 172) |
| | | QAPGKGLEWVAVISYDGGN | NO: 90) | | |
| | | KYYAESVKGRFTISRDNSKN | | | |
| | | TLYLQMNSLRAEDTALFYC | | | |
| | | ARGYDILTGYPDYWGQGTL | | | |
| | | VTVSS (SEQ ID NO: 268) | | | |
| iPS:421387 | HV-79 | QVQLVESGGGVVQPGRSLR | RFAMH | VISFDGGNKYYAESVKG | GYDILTGYPDY |
| | | LSCAASGFTFSRFAMHWVR | (SEQ ID | (SEQ ID NO: 136) | (SEQ ID NO: 172) |
| | | QAPGKGLEWVAVISFDGGN | NO: 88) | | |
| | | KYYAESVKGRFTISRDNSKN | | | |
| | | TLYLQMNSLRAEDTALFYC | | | |
| | | ARGYDILTGYPDYWGQGTL | | | |
| | | VTVSS (SEQ ID NO: 269) | | | |
| iPS:421394 | HV-80 | QVQLVESGGGVVQPGRSLR | RFAMH | VISYDGANKYYAESVKG | GYDILTGYPDY |
| | | LSCAASGFTFSRFAMHWVR | (SEQ ID | (SEQ ID NO: 137) | (SEQ ID NO: 172) |
| | | QAPGKGLEWVAVISYDGAN | NO: 88) | | |
| | | KYYAESVKGRFTISRDNSKN | | | |
| | | TLYLQMNSLRAEDTALFYC | | | |
| | | ARGYDILTGYPDYWGQGTL | | | |
| | | VTVSS (SEQ ID NO: 270) | | | |
| iPS:421855 | HV-81 | QVQLVESGGGVVQPGRSLR | KYAMH | VISFKGSNKYYAESVKG | GYDLLTGYPDY |
| | | LSCAASGFTFSKYAMHWVR | (SEQ ID | (SEQ ID NO: 143) | (SEQ ID NO: 174) |
| | | QAPGKGLEWVAVISFKGSN | NO: 91) | | |
| | | KYYAESVKGRFTISRDNSKN | | | |
| | | TLYLQMNSLRAEDTALFYC | | | |
| | | ARGYDLLTGYPDYWGQGTL | | | |
| | | VTVSS (SEQ ID NO: 271) | | | |
| iPS:421861 | HV-82 | QVQLVESGGGVVQPGRSLR | RYAMH | VISFKGSNKYYAESVKG | GYDLLTGYPDY |
| | | LSCAASGFTFSRYAMHWVR | (SEQ ID | (SEQ ID NO: 109) | (SEQ ID NO: 174) |
| | | QAPGKGLEWVAVISYQGGN | NO: 90) | | |
| | | KYYAESVKGRFTISRDNSKN | | | |
| | | TLYLQMNSLRAEDTALFYC | | | |
| | | ARGYDLLTGYPDYWGQGTL | | | |
| | | VTVSS (SEQ ID NO: 272) | | | |
| iPS:421867 | HV-83 | QVQLVESGGGVVQPGRSLR | HFAMH | VISFSGSNKYYAESVKG | GYDMLTGYPDY |
| | | LSCAASGFTFSHFAMHWVR | (SEQ ID | (SEQ ID NO: 144) | (SEQ ID NO: 175) |
| | | QAPGKGLEWVAVISFSGSNK | NO: 89) | | |
| | | YYAESVKGRFTISRDNSKNT | | | |
| | | LYLQMNSLRAEDTALFYCA | | | |
| | | RGYDMLTGYPDYWGQGTL | | | |
| | | VTVSS (SEQ ID NO: 273) | | | |
| iPS:421873 | HV-84 | QVQLVESGGGVVQPGRSLR | KFAMH | VISYRGGNKYYAESVKG | GYDLLTGYPDY |
| | | LSCAASGFTFSKFAMHWVR | (SEQ ID | (SEQ ID NO: 145) | (SEQ ID NO: 174) |
| | | QAPGKGLEWVAVISYRGGN | NO: 92) | | |
| | | KYYAESVKGRFTISRDNSKN | | | |
| | | TLYLQMNSLRAEDTALFYC | | | |
| | | ARGYDLLTGYPDYWGQGTL | | | |
| | | VTVSS (SEQ ID NO: 274) | | | |
| iPS:421879 | HV-85 | QVQLVESGGGVVQPGRSLR | RYAMH | VISYSGANKYYAESVKG | GYDLLSGYPDY |
| | | LSCAASGFTFSRYAMHWVR | (SEQ ID | (SEQ ID NO: 146) | (SEQ ID NO: 176) |
| | | QAPGKGLEWVAVISYSGAN | NO: 90) | | |
| | | KYYAESVKGRFTISRDNSKN | | | |
| | | TLYLQMNSLRAEDTALFYC | | | |
| | | ARGYDLLSGYPDYWGQGTL | | | |
| | | VTVSS (SEQ ID NO: 275) | | | |
| iPS:421885 | HV-86 | QVQLVESGGGVVQPGRSLR | HYAMH | VISFKGANKYYAESVKG | GYDLLTGYPDY |
| | | LSCAASGFTFSHYAMHWVR | (SEQ ID | (SEQ ID NO: 147) | (SEQ ID NO: 174) |
| | | QAPGKGLEWVAVISFKGAN | NO: 93) | | |
| | | KYYAESVKGRFTISRDNSKN | | | |
| | | TLYLQMNSLRAEDTALFYC | | | |
| | | ARGYDLLTGYPDYWGQGTL | | | |
| | | VTVSS (SEQ ID NO: 276) | | | |
| iPS:421891 | HV-87 | QVQLVESGGGVVQPGRSLR | HYAMH | VISYRGANKYYAESVKG | GYDLLTGYPDY |
| | | LSCAASGFTFSHYAMHWVR | (SEQ ID | (SEQ ID NO: 148) | (SEQ ID NO: 174) |
| | | QAPGKGLEWVAVISYRGAN | NO: 93) | | |
| | | KYYAESVKGRFTISRDNSKN | | | |
| | | TLYLQMNSLRAEDTALFYC | | | |
| | | ARGYDLLTGYPDYWGQGTL | | | |
| | | VTVSS (SEQ ID NO: 277) | | | |
| iPS:421897 | HV-88 | QVQLVESGGGVVQPGRSLR | HYAMH | VISFYGSNKYYAESVKG | GYDFLTGYPDY |
| | | LSCAASGFTFSHYAMHWVR | (SEQ ID | (SEQ ID NO: 149) | (SEQ ID NO: 173) |
| | | QAPGKGLEWVAVISFYGSN | NO: 93) | | |
| | | KYYAESVKGRFTISRDNSKN | | | |
| | | TLYLQMNSLRAEDTALFYC | | | |
| | | ARGYDFLTGYPDYWGQGTL | | | |
| | | VTVSS (SEQ ID NO: 278) | | | |
| iPS:421903 | HV-89 | QVQLVESGGGVVQPGRSLR | HFAMH | VISFFGGNKYYAESVKG | GYDFLTGYPDY |
| | | LSCAASGFTFSHFAMHWVR | (SEQ ID | (SEQ ID NO: 150) | (SEQ ID NO: 173) |
| | | QAPGKGLEWVAVISFFGGN | NO: 89) | | |
| | | KYYAESVKGRFTISRDNSKN | | | |
| | | TLYLQMNSLRAEDTALFYC | | | |
| | | ARGYDFLTGYPDYWGQGTL | | | |
| | | TVSS (SEQ ID NO: 279) | | | |
| iPS:421909 | HV-90 | QVQLVESGGGVVQPGRSLR | HYAMH | VISFMGTNKYYAESVKG | GYDFLTGYPDY |
| | | LSCAASGFTFSHYAMHWVR | (SEQ ID | (SEQ ID NO: 151) | (SEQ ID NO: 173) |
| | | QAPGKGLEWVAVISFMGTN | NO: 93) | | |
| | | KYYAESVKGRFTISRDNSKN | | | |
| | | TLYLQMNSLRAEDTALFYC | | | |
| | | ARGYDFLTGYPDYWGQGTL | | | |
| | | VTVSS (SEQ ID NO: 280) | | | |
| iPS:421915 | HV-91 | QVQLVESGGGVVQPGRSLR | YFAMH | VISHRGTNKYYAESVKG | GYDLLSGYPDY |
| | | LSCAASGFTFSYFAMHWVR | (SEQ ID | (SEQ ID NO: 152) | (SEQ ID NO: 176) |
| | | QAPGKGLEWVAVISHRGTN | NO: 94) | | |
| | | KYYAESVKGRFTISRDNSKN | | | |
| | | TLYLQMNSLRAEDTALFYC | | | |
| | | ARGYDLLSGYPDYWGQGTL | | | |
| | | VTVSS (SEQ ID NO: 281) | | | |
| iPS:480711 | HV-92 | QVQLVESGGGVVQPGRSLR | RFAMH | VINYRGHGKYYAESVKG | GYDVLTGYPDY |
| | | LSCAASGFTFSRFAMHWVR | (SEQ ID | (SEQ ID NO: 153) | (SEQ ID NO: 171) |
| | | QAPGKGLEWVGVINYRGHG | NO: 88) | | |
| | | KYYAESVKGRFTVSRDNSK | | | |
| | | NTLYLQMNSLRAEDTALFY | | | |
| | | CARGYDVLTGYPDYWGQG | | | |
| | | TLVTVSS (SEQ ID NO: 282) | | | |
| iPS:480706 | HV-93 | QVQLVESGGGVVQPGRSLR | RFAMH | VISFSGGSKYYAESVKG | GYDVLTGYPDY |
| | | LSCAASGFTFSRFAMHWVR | (SEQ ID | (SEQ ID NO: 154) | (SEQ ID NO: 171) |
| | | QAPGKGLEWVGVISFSGGSK | NO: 88) | | |
| | | YYAESVKGRFTLSRDNSKNT | | | |
| | | LYLQMNSLRAEDTALFYCA | | | |
| | | RGYDVLTGYPDYWGQGTLV | | | |
| | | TVSS (SEQ ID NO: 283) | | | |
| iPS:480713 | HV-94 | QVQLVESGGGVVQPGRSLR | RFAMH | VISYTGQFKYYAESVKG | GYDVLTGYPDY |
| | | LSCAASGFTFSRFAMHWVR | (SEQ ID | (SEQ ID NO: 155) | (SEQ ID NO: 171) |
| | | QAPGKGLEWVGVISYTGQF | NO: 88) | | |
| | | KYYAESVKGRFTVSRDNSK | | | |
| | | NTLYLQMNSLRAEDTALFY | | | |
| | | CARGYDVLTGYPDYWGQG | | | |
| | | TLVTVSS (SEQ ID NO: 284) | | | |
| iPS:480705 | HV-95 | QVQLVESGGGVVQPGRSLR | RFAMH | VISYTGAQKYYAESVKG | GYDVLTGYPDY |
| | | LSCAASGFTFSRFAMHWVR | (SEQ ID | (SEQ ID NO: 156) | (SEQ ID NO: 171) |
| | | QAPGKGLEWVGVISYTGAQ | NO: 88) | | |
| | | KYYAESVKGRFTMSRDNSK | | | |
| | | NTLYLQMNSLRAEDTALFY | | | |
| | | CARGYDVLTGYPDYWGQG | | | |
| | | TLVTVSS (SEQ ID NO: 285) | | | |
| iPS:48070 | HV-96 | QVQLVESGGGVVQPGRSLR | RFAMH | VISYSGASKYYAESVKG | GYDVLTGYPDY |
| | | LSCAASGFTFSRFAMHWVR | (SEQ ID | (SEQ ID NO: 157) | (SEQ ID NO: 171) |
| | | QAPGKGLEWVGVISYSGAS | NO: 88) | | |
| | | KYYAESVKGRFTMSRDNSK | | | |
| | | NTLYLQMNSLRAEDTALFY | | | |
| | | CARGYDVLTGYPDYWGQG | | | |
| | | TLVTVSS (SEQ ID NO: 286) | | | |
| iPS:480708 | HV-97 | QVQLVESGGGVVQPGRSLR | RFAMH | VISYSGAFKYYAESVKG | GYDVLTGYPDY |
| | | LSCAASGFTFSRFAMHWVR | (SEQ ID | (SEQ ID NO: 158) | (SEQ ID NO: 171) |
| | | QAPGKGLEWVAVISYSGAF | NO: 88) | | |
| | | KYYAESVKGRFTVSRDNSK | | | |
| | | NTLYLQMNSLRAEDTALFY | | | |
| | | CARGYDVLTGYPDYWGQG | | | |
| | | TLVTVSS (SEQ ID NO: 287) | | | |
| iPS:480709 | HV-98 | QVQLVESGGGVVQPGRSLR | RFAMH | VITYTGGAKYYAESVKG | GYDVLTGYPDY |
| | | LSCAASGFTFSRFAMHWVR | (SEQ ID | (SEQ ID NO: 159) | (SEQ ID NO: 171) |
| | | QAPGKGLEWVGVITYTGGA | NO: 88) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 288) | | | |
| iPS:480712 | HV-99 | QVQLVESGGGVVQPGRSLR | RFAMH | VINFQGTTKYYAESVKG | GYDVLTGYPDY |
| | | LSCAASGFTFSRFAMHWVR | (SEQ ID | (SEQ ID NO: 160) | (SEQ ID NO: 171) |
| | | QAPGKGLEWVGVINFQGTT | NO: 88) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 289) | | | |
| iPS:480704 | HV-100 | QVQLVESGGGVVQPGRSLR | RFAMH | VISYSGDLKYYAESVKG | GYDVLTGYPDY |
| | | LSCAASGFTFSRFAMHWVR | (SEQ ID | (SEQ ID NO: 161) | (SEQ ID NO: 171) |
| | | QAPGKGLEWVGVISYSGDL | NO: 88) | | |
| | | KYYAESVKGRFTVSRDNSK | | | |
| | | NTLYLQMNSLRAEDTALFY | | | |
| | | CARGYDVLTGYPDYWGQG | | | |
| | | TLVTVSS (SEQ ID NO: 290) | | | |
| iPS:480710 | HV-101 | QVQLVESGGGVVQPGRSLR | RFAMH | VINYFGDAKYYAESVKG | GYDVLTGYPDY |
| | | LSCAASGFTFSRFAMHWVR | (SEQ ID | (SEQ ID NO: 162) | (SEQ ID NO: 171) |
| | | QAPGKGLEWVGVINYFGDA | NO: 88) | | |
| | | KYYAESVKGRFTISRDNSKN | | | |
| | | TLYLQMNSLRAEDTALFYC | | | |
| | | ARGYDVLTGYPDYWGQGTL | | | |
| | | VTVSS (SEQ ID NO: 291) | | | |
| iPS:480716 | HV-102 | QVQLVESGGGVVQPGRSLR | FYAMH | VISSFGSNKYYAESVKG | GYDLLTGYPDY |
| | | LSCAASGFTFSFYAMHWVR | (SEQ ID | (SEQ ID NO: 163) | (SEQ ID NO: 174) |
| | | QAPGKGLEWVAVISSFGSNK | NO: 95) | | |
| | | YYAESVKGRFTISRDNSKNT | | | |
| | | LYLQMNSLRAEDTALFYCA | | | |
| | | RGYDLLTGYPDYWGQGTLV | | | |
| | | TVSS (SEQ ID NO: 292) | | | |
| iPS:480715 | HV-103 | QVQLVESGGGVVQPGRSLR | YYAMEE | VISYSGSNKYYAESVKG | GYDLLTGYPDY |
| | | LSCAASGFTFSYYAMHWVR | (SEQ ID | (SEQ ID NO: 164) | (SEQ ID NO: 174) |
| | | QAPGKGLEWVAVISYSGSN | NO: 96) | | |
| | | KYYAESVKGRFTISRDNSKN | | | |
| | | TLYLQMNSLRAEDTALFYC | | | |
| | | ARGYDLLTGYPDYWGQGTL | | | |
| | | VTVSS (SEQ ID NO: 293) | | | |
| iPS:480717 | HV-104 | QVQLVESGGGVVQPGRSLR | YYAMEE | VISHYGTNKYYAESVKG | GYDPLTGYPDY |
| | | LSCAASGFTFSYYAMHWVR | (SEQ ID | (SEQ ID NO: 165) | (SEQ ID NO: 177) |
| | | QAPGKGLEWVAVISHYGTN | NO: 96) | | |
| | | KYYAESVKGRFTISRDNSKN | | | |
| | | TLYLQMNSLRAEDTALFYC | | | |
| | | ARGYDPLTGYPDYWGQGTL | | | |
| | | VTVSS (SEQ ID NO: 294) | | | |
| iPS:480714 | HV-105 | QVQLVESGGGVVQPGRSLR | HYAMH | VISYQGSNKYYAESVKG | GYDLLTGYPDY |
| | | LSCAASGFTFSHYAMHWVR | (SEQ ID | (SEQ ID NO: 166) | (SEQ ID NO: 174) |
| | | QAPGKGLEWVAVISYQGSN | NO: 93) | | |
| | | KYYAESVKGRFTISRDNSKN | | | |
| | | TLYLQMNSLRAEDTALFYC | | | |
| | | ARGYDLLTGYPDYWGQGTL | | | |
| | | VTVSS (SEQ ID NO: 295) | | | |

19H8 variants

| | | | | | |
|---|---|---|---|---|---|
| 19H8 | HV-106 | QVQLQQSGPGLVKPSQTLSL | SNSATWN | RTYYRSKWSNHYAVSVKS | GTWKQLWFLDH |
| | | TCAISGDSVSSNSATWNWIR | (SEQ ID | (SEQ ID NO: 167) | (SEQ ID NO: 178) |
| | | QSPSRGLEWLGRTYYRSKW | NO: 97) | | |
| | | SNHYAVSVKSRITINPDTSKS | | | |
| | | QFSLQLNSVTPEDTAVYYCA | | | |
| | | RGTWKQLWFLDHWGQGTL | | | |
| | | VTVSS (SEQ ID NO: 296) | | | |
| iPS:448202 | HV-107 | QVQLQQSGPGLVKPSQTLSL | NRLATWN | RTYYRGKWKNHYAVSVKS | GTWNQDWFLDH |
| | | TCAISGDSVSNRLATWNWIR | (SEQ ID | (SEQ ID NO: 168) | (SEQ ID NO: 179) |
| | | QSPSRGLEWLGRTYYRGKW | NO: 98) | | |
| | | KNHYAVSVKSRITINPDTSKS | | | |
| | | QFSLQLNSVTPEDTAVYYCA | | | |
| | | RGTWNQDWFLDHWGQGTL | | | |
| | | VTVSS (SEQ ID NO: 297) | | | |
| iPS:449375 | HV-108 | QVQLQQSGPGLVKPSQTLSL | NRLATWN | RTYYRGKWKNHYAVSVKS | GTWDQDWFLDH |
| | | TCAISGDSVSNRLATWNWIR | (SEQ ID | (SEQ ID NO: 168) | (SEQ ID NO: 180) |
| | | QSPSRGLEWLGRTYYRGKW | NO: 98) | | |
| | | KNHYAVSVKSRITINPDTSKS | | | |
| | | QFSLQLNSVTPEDTAVYYCA | | | |
| | | RGTWDQDWFLDHWGQGTL | | | |
| | | VTVSS (SEQ ID NO: 298) | | | |
| iPS:448083 | HV-109 | QVQLQQSGPGLVKPSQTLSL | SRQATWN | RTYYRGKWKNHYAVSVKS | GTWEQDWFLDH |
| | | TCAISGDSVSSRQATWNWIR | (SEQ ID | (SEQ ID NO: 168) | (SEQ ID NO: 181) |
| | | QSPSRGLEWLGRTYYRGKW | NO: 99) | | |
| | | KNHYAVSVKSRITINPDTSKS | | | |
| | | QFSLQLNSVTPEDTAVYYCA | | | |
| | | RGTWEQDWFLDHWGQGTL | | | |
| | | VTVSS (SEQ ID NO: 299) | | | |
| iPS:452128 | HV-110 | QVQLQQSGPGLVKPSQTLSL | NKQATWN | RTYYRGKWKNHYAVSVKS | GMWNQNWFLDH |
| | | TCAISGDSVSNKQATWNWIR | (SEQ ID | (SEQ ID NO: 168) | (SEQ ID NO: 182) |
| | | QSPSRGLEWLGRTYYRGKW | NO: 100) | | |
| | | KNHYAVSVKSRITINPDTSKS | | | |
| | | QFSLQLNSVTPEDTAVYYCA | | | |
| | | RGMWNQNWFLDHWGQGTL | | | |
| | | VTVSS (SEQ ID NO: 300) | | | |
| iPS:448195 | HV-110 | QVQLQQSGPGLVKPSQTLSL | NKQATWN | RTYYRGKWKNHYAVSVKS | GMWNQNWFLDH |
| | | TCAISGDSVSNKQATWNWIR | (SEQ ID | (SEQ ID NO: 168) | (SEQ ID NO: 182) |
| | | QSPSRGLEWLGRTYYRGKW | NO: 100) | | |
| | | KNHYAVSVKSRITINPDTSKS | | | |
| | | QFSLQLNSVTPEDTAVYYCA | | | |
| | | RGMWNQNVVFLDHWGQGTL | | | |
| | | VTVSS (SEQ ID NO: 300) | | | |
| iPS:448466 | HV-111 | QVQLQQSGPGLVKPSQTLSL | NKQATWN | RTYYRGQWKNHYAVSVKS | GTWIGDWFMDH |
| | | TCAISGDSVSNKQATWNWIR | (SEQ ID | (SEQ ID NO: 169) | (SEQ ID NO: 183) |
| | | QSPSRGLEWLGRTYYRGQW | NO: 100) | | |
| | | KNHYAVSVKSRITINPDTSKS | | | |
| | | QFSLQLNSVTPEDTAVYYCA | | | |
| | | RGTWIGDWFMDHWGQGTL | | | |
| | | VTVSS (SEQ ID NO: 301) | | | |
| iPS:448689 | HV-107 | QVQLQQSGPGLVKPSQTLSL | NRLATWN | RTYYRGKWKNHYAVSVKS | GTWNQDWFLDH |
| | | TCAISGDSVSNRLATWNWIR | (SEQ ID | (SEQ ID NO: 168) | (SEQ ID NO: 179) |
| | | QSPSRGLEWLGRTYYRGKW | NO: 98) | | |
| | | KNHYAVSVKSRITINPDTSKS | | | |
| | | QFSLQLNSVTPEDTAVYYCA | | | |
| | | RGTWNQDWFLDHWGQGTL | | | |
| | | VTVSS (SEQ ID NO: 297) | | | |
| iPS:449034 | HV-112 | QVQLQQSGPGLVKPSQTLSL | NKQATWN | RTYYRGKWKNHYAVSVKS | GTWIQDWFLDH |
| | | TCAISGDSVSNKQATWNWIR | (SEQ ID | (SEQ ID NO: 168) | (SEQ ID NO: 184) |
| | | QSPSRGLEWLGRTYYRGKW | NO: 100) | | |
| | | KNHYAVSVKSRITINPDTSKS | | | |
| | | QFSLQLNSVTPEDTAVYYCA | | | |
| | | RGTWIQDWFLDHWGQGTL | | | |
| | | VTVSS (SEQ ID NO: 302) | | | |
| iPS:448075 | HV-113 | QVQLQQSGPGLVKPSQTLSL | SNHATWN | RTYYRGKWKNHYAVSVKS | GTWDQDWFLDH |
| | | TCAISGDSVSSNHATWNWIR | (SEQ ID | (SEQ ID NO: 168) | (SEQ ID NO: 180) |
| | | QSPSRGLEWLGRTYYRGKW | NO: 101) | | |
| | | KNHYAVSVKSRITINPDTSKS | | | |
| | | QFSLQLNSVTPEDTAVYYCA | | | |
| | | RGTWDQDWFLDHWGQGTL | | | |
| | | VTVSS (SEQ ID NO: 303) | | | |
| iPS:448924 | HV-114 | QVQLQQSGPGLVKPSQTLSL | SRYATWN | RTYYRGQWKNHYAVSVKS | GMWNQNWFLDH |
| | | TCAISGDSVSSRYATWNWIR | (SEQ ID | (SEQ ID NO: 169) | (SEQ ID NO: 182) |
| | | QSPSRGLEWLGRTYYRGQW | NO: 102) | | |
| | | KNHYAVSVKSRITINPDTSKS | | | |
| | | QFSLQLNSVTPEDTAVYYCA | | | |
| | | RGMWNQNWFLDHWGQGTL | | | |
| | | VTVSS (SEQ ID NO: 304) | | | |
| iPS:448752 | HV-110 | QVQLQQSGPGLVKPSQTLSL | NKQATWN | RTYYRGKWKNHYAVSVKS | GMWNQNWFLDH |
| | | TCAISGDSVSNKQATWNWIR | (SEQ ID | (SEQ ID NO: 168) | (SEQ ID NO: 182) |
| | | QSPSRGLEWLGRTYYRGKW | NO: 100) | | |
| | | KNHYAVSVKSRITINPDTSKS | | | |
| | | QFSLQLNSVTPEDTAVYYCA | | | |
| | | RGMWNQNWFLDHWGQGTL | | | |
| | | VTVSS (SEQ ID NO: 300) | | | |
| iPS:448772 | HV-115 | QVQLQQSGPGLVKPSQTLSL | NKQATWN | RTYYRGKWKNHYAVSVKS | GMWSGDWFLDH |
| | | TCAISGDSVSNKQATWNWIR | (SEQ ID | (SEQ ID NO: 168) | (SEQ ID NO: 185) |
| | | QSPSRGLEWLGRTYYRGKW | NO: 100) | | |
| | | KNHYAVSVKSRITINPDTSKS | | | |
| | | QFSLQLNSVTPEDTAVYYCA | | | |
| | | RGMWSGDWFLDHWGQGTL | | | |
| | | VTVSS (SEQ ID NO: 305) | | | |
| iPS:448117 | HV-116 | QVQLQQSGPGLVKPSQTLSL | SHVATWN | RTYYRGKWKNHYAVSVKS | GMWSEDWFLDH |
| | | TCAISGDSVSSHVATWNWIR | (SEQ ID | (SEQ ID NO: 168) | (SEQ ID NO: 186) |
| | | QSPSRGLEWLGRTYYRGKW | NO: 103) | | |
| | | KNHYAVSVKSRITINPDTSKS | | | |
| | | QFSLQLNSVTPEDTAVYYCA | | | |
| | | RGMWSEDWFLDHWGQGTL | | | |
| | | VTVSS (SEQ ID NO: 306) | | | |
| iPS:448788 | HV-117 | QVQLQQSGPGLVKPSQTLSL | SRQATWN | RTYYRGKWKNHYAVSVKS | GMWQGNWFLDH |
| | | TCAISGDSVSSRQATWNWIR | (SEQ ID | (SEQ ID NO: 168) | (SEQ ID NO: 187) |
| | | QSPSRGLEWLGRTYYRGKW | NO: 99) | | |
| | | KNHYAVSVKSRITINPDTSKS | | | |
| | | QFSLQLNSVTPEDTAVYYCA | | | |
| | | RGMWQGNWFLDHWGQGTL | | | |
| | | VTVSS (SEQ ID NO: 307) | | | |
| iPS:448593 | HV-118 | QVQLQQSGPGLVKPSQTLSL | NHQATWN | RTYYRGKWKNHYAVSVKS | GTWIQDWFLDH |
| | | TCAISGDSVSNHQATWNWIR | (SEQ ID | (SEQ ID NO: 168) | (SEQ ID NO: 184) |
| | | QSPSRGLEWLGRTYYRGKW | NO: 104) | | |
| | | KNHYAVSVKSRITINPDTSKS | | | |
| | | QFSLQLNSVTPEDTAVYYCA | | | |
| | | RGTWIQDWFLDHWGQGTL | | | |
| | | VTVSS (SEQ ID NO: 308) | | | |
| iPS:448238 | HV-119 | QVQLQQSGPGLVKPSQTLSL | SRDATWN | RTYYRGKWKNHYAVSVKS | GQWNEDWFLDH |
| | | TCAISGDSVSSRDATWNWIR | (SEQ ID | (SEQ ID NO: 168) | (SEQ ID NO: 188) |
| | | QSPSRGLEWLGRTYYRGKW | NO: 105) | | |
| | | KNHYAVSVKSRITINPDTSKS | | | |
| | | QFSLQLNSVTPEDTAVYYCA | | | |
| | | RGQWNEDWFLDHWGQGTL | | | |
| | | VTVSS (SEQ ID NO: 309) | | | |
| iPS:448901 | HV-120 | QVQLQQSGPGLVKPSQTLSL | NRLATWN | RTYYRGKWKNHYAVSVKS | GRWEGDWFFDH |
| | | TCAISGDSVSNRLATWNWIR | (SEQ ID | (SEQ ID NO: 168) | (SEQ ID NO: 189) |
| | | QSPSRGLEWLGRTYYRGKW | NO: 98) | | |
| | | KNHYAVSVKSRITINPDTSKS | | | |
| | | QFSLQLNSVTPEDTAVYYCA | | | |
| | | RGRWEGDWFFDHWGQGTL | | | |
| | | VTVSS (SEQ ID NO: 310) | | | |
| iPS:448655 | HV-121 | QVQLQQSGPGLVKPSQTLSL | NKQATWN | RTYFRRTWKNHYAVSVKS | GMWSEDWFLDH |
| | | TCAISGDSVSNKQATWNWIR | (SEQ ID | (SEQ ID NO: 170) | (SEQ ID NO: 186) |
| | | QSPSRGLEWLGRTYFRRTW | NO: 100) | | |
| | | KNHYAVSVKSRITINPDTSKS | | | |
| | | QFSLQLNSVTPEDTAVYYCA | | | |
| | | RGMWSEDWFLDHWGQGTL | | | |
| | | VTVSS (SEQ ID NO: 311) | | | |
| iPS:448730 | HV-122 | QVQLQQSGPGLVKPSQTLSL | NRLATWN | RTYYRGKWKNHYAVSVKS | GVWIGNWFLDH |
| | | TCAISGDSVSNRLATWNWIR | (SEQ ID | (SEQ ID NO: 168) | (SEQ ID NO: 190) |
| | | QSPSRGLEWLGRTYYRGKW | NO: 98) | | |
| | | KNHYAVSVKSRITINPDTSKS | | | |
| | | QFSLQLNSVTPEDTAVYYCA | | | |
| | | RGVWIGNWFLDHWGQGTL | | | |
| | | VTVSS (SEQ ID NO: 312) | | | |
| iPS:449027 | HV-110 | QVQLQQSGPGLVKPSQTLSL | NKQATWN | RTYYRGKWKNHYAVSVKS | GMWNQNWFLDH |
| | | TCAISGDSVSNKQATWNWIR | (SEQ ID | (SEQ ID NO: 168) | (SEQ ID NO: 182) |
| | | QSPSRGLEWLGRTYYRGKW | NO: 100) | | |
| | | KNHYAVSVKSRITINPDTSKS | | | |
| | | QFSLQLNSVTPEDTAVYYCA | | | |
| | | RGMWNQNWFLDHWGQGTL | | | |
| | | VTVSS (SEQ ID NO: 300) | | | |
| 3574 | HV-110 | QVQLQQSGPGLVKPSQTLSL | NKQATWN | RTYYRGKWKNHYAVSVKS | GMWNQNWFLDH |
| | | TCAISGDSVSNKQATWNWIR | (SEQ ID | (SEQ ID NO: 168) | (SEQ ID NO: 182) |
| | | QSPSRGLEWLGRTYYRGKW | NO: 100) | | |
| | | KNHYAVSVKSRITINPDTSKS | | | |
| | | QFSLQLNSVTPEDTAVYYCA | | | |
| | | RGMWNQNWFLDHWGQGTL | | | |
| | | VTVSS (SEQ ID NO: 300) | | | |
| 3575 | HV-106 | QVQLQQSGPGLVKPSQTLSL | SNSATWN | RTYYRSKWSNHYAVSVKS | GTWKQLWFLDH |
| | | TCAISGDSVSSNSATWNWIR | (SEQ ID | (SEQ ID NO: 167) | (SEQ ID NO: 178) |
| | | QSPSRGLEWLGRTYYRSKW | NO: 97) | | |
| | | SNHYAVSVKSRITINPDTSKS | | | |
| | | QFSLQLNSVTPEDTAVYYCA | | | |
| | | RGTWKQLWFLDHWGQGTL | | | |
| | | VTVSS (SEQ ID NO: 296) | | | |

The anti-PAC1 antibodies or antigen-binding fragments of the invention may comprise one or more of the light chain CDRs (i.e. CDRLs) and/or heavy chain CDRs (i.e. CDRHs) presented in Tables 1A and 1B, respectively. In some embodiments, the anti-PAC1 antibodies or binding fragments thereof are derived from antibody 29G4v9, 29G4v10, or 29G4v22 (i.e. has one or more substitutions in one or more of the CDRLs and/or CDRHs of the 29G4v9, 29G4v10, or 29G4v22 antibody). For instance, in certain embodiments, the anti-PAC1 antibodies or antigen-binding fragments comprise one or more light chain CDRs selected from (i) a CDRL1 selected from SEQ ID NOs: 5 to 16, (ii) a CDRL2 of SEQ ID NO: 26, and (iii) a CDRL3 selected from SEQ ID NOs: 36 to 38, and (iv) a CDRL of (i), (ii) and (iii) that contains one or more, e.g., one, two, three, four or more amino acid substitutions (e.g., conservative amino acid substitutions), deletions or insertions of no more than five, four, three, two, or one amino acids. In these and other embodiments, the anti-PAC1 antibodies or antigen-binding fragments comprise one or more heavy chain CDRs selected from (i) a CDRH1 selected from SEQ ID NOs: 88 to 96, (ii) a CDRH2 selected from SEQ ID NOs: 106 to 166, and (iii) a CDRH3 selected from SEQ ID NOs: 171 to 177, and (iv) a CDRH of (i), (ii) and (iii) that contains one or more, e.g., one, two, three, four or more amino acid substitutions (e.g., conservative amino acid substitutions), deletions or insertions of no more than five, four, three, two, or one amino acids amino acids.

In other embodiments, the anti-PAC1 antibodies or binding fragments thereof are derived from antibody 19H8 (i.e. has one or more substitutions in one or more of the CDRLs and/or CDRHs of the 19H8 antibody). In such embodiments, the anti-PAC1 antibodies or antigen-binding fragments comprise one or more light chain CDRs selected from (i) a CDRL1 selected from SEQ ID NOs: 17 to 25, (ii) a CDRL2 selected from SEQ ID NOs: 27 to 35, and (iii) a CDRL3 selected from SEQ ID NOs: 39 to 51, and (iv) a CDRL of (i), (ii) and (iii) that contains one or more, e.g., one, two, three, four or more amino acid substitutions (e.g., conservative amino acid substitutions), deletions or insertions of no more than five, four, three, two, or one amino acids. In these and other embodiments, the anti-PAC1 antibodies or antigen-binding fragments comprise one or more heavy chain CDRs selected from (i) a CDRH1 selected from SEQ ID NOs: 97 to 105, (ii) a CDRH2 selected from SEQ ID NOs: 167 to 170, and (iii) a CDRH3 selected from SEQ ID NOs: 178 to 190, and (iv) a CDRH of (i), (ii) and (iii) that contains one or more, e.g., one, two, three, four or more amino acid substitutions (e.g., conservative amino acid substitutions), deletions or insertions of no more than five, four, three, two, or one amino acids amino acids.

In certain embodiments, the anti-PAC1 antibodies or antigen-binding fragments may comprise 1, 2, 3, 4, 5, or 6 variant forms of the CDRs listed in Tables 1A and 1B, each having at least 80%, 85%, 90% or 95% sequence identity to a CDR sequence listed in Tables 1A and 1B. In some embodiments, the anti-PAC1 antibodies or antigen-binding fragments include 1, 2, 3, 4, 5, or 6 of the CDRs listed in Tables 1A and 1B, each differing by no more than 1, 2, 3, 4 or 5 amino acids from the CDRs listed in these tables. In some embodiments, the anti-PAC1 antibodies or antigen-binding fragments of the invention comprise a CDRL1 comprising a sequence selected from SEQ ID NOs: 5 to 16 or a variant thereof having one, two, three or four amino acid substitutions; a CDRL2 comprising the sequence of SEQ ID NO: 26 or a variant thereof having one, two, three or four amino acid substitutions; a CDRL3 comprising a sequence selected from SEQ ID NOs: 36 to 38 or a variant thereof having one, two, three or four amino acid substitutions; a CDRH1 comprising a sequence selected from SEQ ID NOs: 88 to 96 or a variant thereof having one, two, three or four amino acid substitutions; a CDRH2 comprising a sequence selected from SEQ ID NOs: 106 to 166 or a variant thereof having one, two, three or four amino acid substitutions; and a CDRH3 comprising a sequence selected from SEQ ID NOs: 171 to 177 or a variant thereof having one, two, three or four amino acid substitutions. In other embodiments, the anti-PAC1 antibodies or antigen-binding fragments of the invention comprise a CDRL1 comprising a sequence selected from SEQ ID NOs: 5 to 16; a CDRL2 comprising the sequence of SEQ ID NO: 26; a CDRL3 comprising a sequence selected from SEQ ID NOs: 36 to 38; a CDRH1 comprising a sequence selected from SEQ ID NOs: 88 to 96; a CDRH2 comprising a sequence selected from SEQ ID NOs: 106 to 166; and a CDRH3 comprising a sequence selected from SEQ ID NOs: 171 to 177.

In other embodiments, the anti-PAC1 antibodies or antigen-binding fragments of the invention comprise a CDRL1 comprising a sequence selected from SEQ ID NOs: 17 to 25 or a variant thereof having one, two, three or four amino acid substitutions; a CDRL2 comprising a sequence selected from SEQ ID NOs: 27 to 35 or a variant thereof having one, two, three or four amino acid substitutions; a CDRL3 comprising a sequence selected from SEQ ID NOs: 39 to 51 or a variant thereof having one, two, three or four amino acid substitutions; a CDRH1 comprising a sequence selected from SEQ ID NOs: 97 to 105 or a variant thereof having one, two, three or four amino acid substitutions; a CDRH2 comprising a sequence selected from SEQ ID NOs: 167 to 170 or a variant thereof having one, two, three or four amino acid substitutions; and a CDRH3 comprising a sequence selected from SEQ ID NOs: 178 to 190 or a variant thereof having one, two, three or four amino acid substitutions. In other embodiments, the anti-PAC1 antibodies or antigen-binding fragments of the invention comprise a CDRL1 comprising a sequence selected from SEQ ID NOs: 17 to 25; a CDRL2 comprising a sequence selected from SEQ ID NOs: 27 to 35; a CDRL3 comprising a sequence selected from SEQ ID NOs: 39 to 51; a CDRH1 comprising a sequence selected from SEQ ID NOs: 97 to 105; a CDRH2 comprising a sequence selected from SEQ ID NOs: 167 to 170; and a CDRH3 comprising a sequence selected from SEQ ID NOs: 178 to 190.

In particular embodiments, the anti-PAC1 antibodies or antigen-binding fragments of the invention comprise a light chain variable region comprising a CDRL1, a CDRL2, and a CDRL3, wherein: (a) CDRL1, CDRL2, and CDRL3 have the sequence of SEQ ID NOs: 7, 26, and 36, respectively; (b) CDRL1, CDRL2, and CDRL3 have the sequence of SEQ ID NOs: 5, 26, and 36, respectively; (c) CDRL1, CDRL2, and CDRL3 have the sequence of SEQ ID NOs: 12, 26, and 36, respectively; or (d) CDRL1, CDRL2, and CDRL3 have the sequence of SEQ ID NOs: 13, 26, and 36, respectively.

In other particular embodiments, the anti-PAC1 antibodies or antigen-binding fragments of the invention comprise a heavy chain variable region comprising a CDRH1, a CDRH2, and a CDRH3, wherein: (a) CDRH1, CDRH2, and CDRH3 have the sequence of SEQ ID NOs: 88, 108, and 171, respectively; (b) CDRH1, CDRH2, and CDRH3 have the sequence of SEQ ID NOs: 88, 116, and 171, respectively; (c) CDRH1, CDRH2, and CDRH3 have the sequence of SEQ ID NOs: 88, 134, and 171, respectively; (d) CDRH1, CDRH2, and CDRH3 have the sequence of SEQ ID NOs: 92, 145, and 174, respectively; (e) CDRH1, CDRH2, and CDRH3 have the sequence of SEQ ID NOs: 88, 108, and 172, respectively; (f) CDRH1, CDRH2, and CDRH3 have the sequence of SEQ ID NOs: 88, 128, and 172, respectively; (g) CDRH1, CDRH2, and CDRH3 have the sequence of SEQ ID NOs: 88, 153, and 171, respectively; (i) CDRH1, CDRH2, and CDRH3 have the sequence of SEQ ID NOs: 88, 154, and 171, respectively; or (j) CDRH1, CDRH2, and CDRH3 have the sequence of SEQ ID NOs: 88, 155, and 171, respectively.

In certain embodiments, the anti-PAC1 antibodies or antigen-binding fragments of the invention comprise a light chain variable region comprising a CDRL1, a CDRL2, and a CDRL3 and a heavy chain variable region comprising a CDRH1, a CDRH2, and a CDRH3, wherein:
(a) CDRL1, CDRL2, and CDRL3 have the sequence of SEQ ID NOs: 7, 26, and 36, respectively, and CDRH1, CDRH2, and CDRH3 have the sequence of SEQ ID NOs: 88, 108, and 171, respectively;
(b) CDRL1, CDRL2, and CDRL3 have the sequence of SEQ ID NOs: 7, 26, and 36, respectively, and CDRH1, CDRH2, and CDRH3 have the sequence of SEQ ID NOs: 88, 116, and 171, respectively;
(c) CDRL1, CDRL2, and CDRL3 have the sequence of SEQ ID NOs: 5, 26, and 36, respectively, and CDRH1, CDRH2, and CDRH3 have the sequence of SEQ ID NOs: 88, 134, and 171, respectively;
(d) CDRL1, CDRL2, and CDRL3 have the sequence of SEQ ID NOs: 5, 26, and 36, respectively, and CDRH1, CDRH2, and CDRH3 have the sequence of SEQ ID NOs: 92, 145, and 174, respectively;
(e) CDRL1, CDRL2, and CDRL3 have the sequence of SEQ ID NOs: 7, 26, and 36, respectively, and CDRH1, CDRH2, and CDRH3 have the sequence of SEQ ID NOs: 88, 108, and 172, respectively;
(f) CDRL1, CDRL2, and CDRL3 have the sequence of SEQ ID NOs: 5, 26, and 36, respectively, and CDRH1, CDRH2, and CDRH3 have the sequence of SEQ ID NOs: 88, 128, and 172, respectively;
(g) CDRL1, CDRL2, and CDRL3 have the sequence of SEQ ID NOs: 7, 26, and 36, respectively, and CDRH1, CDRH2, and CDRH3 have the sequence of SEQ ID NOs: 88, 134, and 171, respectively;
(h) CDRL1, CDRL2, and CDRL3 have the sequence of SEQ ID NOs: 12, 26, and 36, respectively, and CDRH1, CDRH2, and CDRH3 have the sequence of SEQ ID NOs: 88, 153, and 171, respectively;
(i) CDRL1, CDRL2, and CDRL3 have the sequence of SEQ ID NOs: 12, 26, and 36, respectively, and CDRH1, CDRH2, and CDRH3 have the sequence of SEQ ID NOs: 88, 154, and 171, respectively; or
(j) CDRL1, CDRL2, and CDRL3 have the sequence of SEQ ID NOs: 13, 26, and 36, respectively, and CDRH1, CDRH2, and CDRH3 have the sequence of SEQ ID NOs: 88, 155, and 171, respectively.

In one embodiment, the anti-PAC1 antibody or antigen-binding fragment thereof comprises a light chain variable region comprising a CDRL1, a CDRL2, and a CDRL3 and a heavy chain variable region comprising a CDRH1, a CDRH2, and a CDRH3, wherein CDRL1, CDRL2, and CDRL3 have the sequence of SEQ ID NOs: 7, 26, and 36, respectively, and CDRH1, CDRH2, and CDRH3 have the sequence of SEQ ID NOs: 88, 108, and 171, respectively. In another embodiment, the anti-PAC1 antibody or antigen-binding fragment thereof comprises a light chain variable region comprising a CDRL1, a CDRL2, and a CDRL3 and a heavy chain variable region comprising a CDRH1, a CDRH2, and a CDRH3, wherein CDRL1, CDRL2, and CDRL3 have the sequence of SEQ ID NOs: 7, 26, and 36, respectively, and CDRH1, CDRH2, and CDRH3 have the sequence of SEQ ID NOs: 88, 116, and 171, respectively. In yet another embodiment, the anti-PAC1 antibody or antigen-binding fragment thereof comprises a light chain variable region comprising a CDRL1, a CDRL2, and a CDRL3 and a heavy chain variable region comprising a CDRH1, a CDRH2, and a CDRH3, wherein CDRL1, CDRL2, and CDRL3 have the sequence of SEQ ID NOs: 5, 26, and 36, respectively, and CDRH1, CDRH2, and CDRH3 have the sequence of SEQ ID NOs: 88, 134, and 171, respectively. In still another embodiment, the anti-PAC1 antibody or antigen-binding fragment thereof comprises a light chain variable region comprising a CDRL1, a CDRL2, and a CDRL3 and a heavy chain variable region comprising a CDRH1, a CDRH2, and a CDRH3, wherein CDRL1, CDRL2, and CDRL3 have the sequence of SEQ ID NOs: 5, 26, and 36, respectively, and CDRH1, CDRH2, and CDRH3 have the sequence of SEQ ID NOs: 92, 145, and 174, respectively. In one particular embodiment, the anti-PAC1 antibody or antigen-binding fragment thereof comprises a light chain variable region comprising a CDRL1, a CDRL2, and a CDRL3 and a heavy chain variable region comprising a CDRH1, a CDRH2, and a CDRH3, wherein CDRL1, CDRL2, and CDRL3 have the sequence of SEQ ID NOs: 7, 26, and 36, respectively, and CDRH1, CDRH2, and CDRH3 have the sequence of SEQ ID NOs: 88, 108, and 172, respectively. In another particular embodiment, the anti-PAC1 antibody or antigen-binding fragment thereof comprises a light chain variable region comprising a CDRL1, a CDRL2, and a CDRL3 and a heavy chain variable region comprising a CDRH1, a CDRH2, and a CDRH3, wherein CDRL1, CDRL2, and CDRL3 have the sequence of SEQ ID NOs: 12, 26, and 36, respectively, and CDRH1, CDRH2, and CDRH3 have the sequence of SEQ ID NOs: 88, 153, and 171, respectively.

In certain embodiments, the anti-PAC1 antibodies or antigen-binding fragments of the invention comprise a light chain variable region comprising a CDRL1, a CDRL2, and a CDRL3, wherein: (a) CDRL1, CDRL2, and CDRL3 have the sequence of SEQ ID NOs: 25, 31, and 42, respectively; (b) CDRL1, CDRL2, and CDRL3 have the sequence of SEQ ID NOs: 20, 30, and 44, respectively; (c) CDRL1, CDRL2, and CDRL3 have the sequence of SEQ ID NOs: 17, 33, and 42, respectively; (d) CDRL1, CDRL2, and CDRL3 have the sequence of SEQ ID NOs: 23, 31, and 50, respectively; (e) CDRL1, CDRL2, and CDRL3 have the sequence of SEQ ID NOs: 17, 31, and 44, respectively; (f) CDRL1, CDRL2, and CDRL3 have the sequence of SEQ ID NOs: 17, 27, and 39, respectively; (g) CDRL1, CDRL2, and CDRL3 have the sequence of SEQ ID NOs: 18, 31, and 46, respectively; (h) CDRL1, CDRL2, and CDRL3 have the sequence of SEQ ID NOs: 17, 28, and 40, respectively; (i) CDRL1, CDRL2, and CDRL3 have the sequence of SEQ ID NOs: 18, 30, and 43, respectively; or (j) CDRL1, CDRL2, and CDRL3 have the sequence of SEQ ID NOs: 22, 28, and 49, respectively.

In certain other embodiments, the anti-PAC1 antibodies or antigen-binding fragments of the invention comprise a heavy chain variable region comprising a CDRH1, a CDRH2, and a CDRH3, wherein: (a) CDRH1, CDRH2, and CDRH3 have the sequence of SEQ ID NOs: 98, 168, and 190, respectively; (b) CDRH1, CDRH2, and CDRH3 have the sequence of SEQ ID NOs: 100, 168, and 182, respectively; (c) CDRH1, CDRH2, and CDRH3 have the sequence of SEQ ID NOs: 99, 168, and 187, respectively; (d) CDRH1, CDRH2, and CDRH3 have the sequence of SEQ ID NOs: 97, 167, and 178, respectively; (e) CDRH1, CDRH2, and CDRH3 have the sequence of SEQ ID NOs: 98, 168, and 189, respectively; (f) CDRH1, CDRH2, and CDRH3 have the sequence of SEQ ID NOs: 98, 168, and 179, respectively; or (g) CDRH1, CDRH2, and CDRH3 have the sequence of SEQ ID NOs: 102, 169, and 182, respectively.

In some embodiments, the anti-PAC1 antibodies or antigen-binding fragments of the invention comprise a light chain variable region comprising a CDRL1, a CDRL2, and a CDRL3 and a heavy chain variable region comprising a CDRH1, a CDRH2, and a CDRH3, wherein:
(a) CDRL1, CDRL2, and CDRL3 have the sequence of SEQ ID NOs: 25, 31, and 42, respectively, and CDRH1, CDRH2, and CDRH3 have the sequence of SEQ ID NOs: 98, 168, and 190, respectively;
(b) CDRL1, CDRL2, and CDRL3 have the sequence of SEQ ID NOs: 20, 30, and 44, respectively, and CDRH1, CDRH2, and CDRH3 have the sequence of SEQ ID NOs: 100, 168, and 182, respectively;
(c) CDRL1, CDRL2, and CDRL3 have the sequence of SEQ ID NOs: 17, 33, and 42, respectively, and CDRH1, CDRH2, and CDRH3 have the sequence of SEQ ID NOs: 99, 168, and 187, respectively;
(d) CDRL1, CDRL2, and CDRL3 have the sequence of SEQ ID NOs: 23, 31, and 50, respectively, and CDRH1, CDRH2, and CDRH3 have the sequence of SEQ ID NOs: 97, 167, and 178, respectively;
(e) CDRL1, CDRL2, and CDRL3 have the sequence of SEQ ID NOs: 17, 31, and 44, respectively, and CDRH1, CDRH2, and CDRH3 have the sequence of SEQ ID NOs: 98, 168, and 189, respectively;
(f) CDRL1, CDRL2, and CDRL3 have the sequence of SEQ ID NOs: 17, 27, and 39, respectively, and CDRH1, CDRH2, and CDRH3 have the sequence of SEQ ID NOs: 100, 168, and 182, respectively;
(g) CDRL1, CDRL2, and CDRL3 have the sequence of SEQ ID NOs: 18, 31, and 46, respectively, and CDRH1, CDRH2, and CDRH3 have the sequence of SEQ ID NOs: 98, 168, and 179, respectively;
(h) CDRL1, CDRL2, and CDRL3 have the sequence of SEQ ID NOs: 17, 28, and 40, respectively, and CDRH1, CDRH2, and CDRH3 have the sequence of SEQ ID NOs: 98, 168, and 179, respectively;
(i) CDRL1, CDRL2, and CDRL3 have the sequence of SEQ ID NOs: 18, 30, and 43, respectively, and CDRH1, CDRH2, and CDRH3 have the sequence of SEQ ID NOs: 100, 168, and 182, respectively; or
(j) CDRL1, CDRL2, and CDRL3 have the sequence of SEQ ID NOs: 22, 28, and 49, respectively, and CDRH1, CDRH2, and CDRH3 have the sequence of SEQ ID NOs: 102, 169, and 182, respectively.

In one embodiment, the anti-PAC1 antibody or antigen-binding fragment thereof comprises a light chain variable region comprising a CDRL1, a CDRL2, and a CDRL3 and a heavy chain variable region comprising a CDRH1, a CDRH2, and a CDRH3, wherein CDRL1, CDRL2, and CDRL3 have the sequence of SEQ ID NOs: 25, 31, and 42, respectively, and CDRH1, CDRH2, and CDRH3 have the sequence of SEQ ID NOs: 98, 168, and 190, respectively. In another embodiment, the anti-PAC1 antibody or antigen-binding fragment thereof comprises a light chain variable region comprising a CDRL1, a CDRL2, and a CDRL3 and a heavy chain variable region comprising a CDRH1, a CDRH2, and a CDRH3, wherein CDRL1, CDRL2, and CDRL3 have the sequence of SEQ ID NOs: 20, 30, and 44, respectively, and CDRH1, CDRH2, and CDRH3 have the sequence of SEQ ID NOs: 100, 168, and 182, respectively. In yet another embodiment, the anti-PAC1 antibody or antigen-binding fragment thereof comprises a light chain variable region comprising a CDRL1, a CDRL2, and a CDRL3 and a heavy chain variable region comprising a CDRH1, a CDRH2, and a CDRH3, wherein CDRL1, CDRL2, and CDRL3 have the sequence of SEQ ID NOs: 17, 33, and 42, respectively, and CDRH1, CDRH2, and CDRH3 have the sequence of SEQ ID NOs: 99, 168, and 187, respectively. In still another embodiment, the anti-PAC1 antibody or antigen-binding fragment thereof comprises a light chain variable region comprising a CDRL1, a CDRL2, and a CDRL3 and a heavy chain variable region comprising a CDRH1, a CDRH2, and a CDRH3, wherein CDRL1, CDRL2, and CDRL3 have the sequence of SEQ ID NOs: 23, 31, and 50, respectively, and CDRH1, CDRH2, and CDRH3 have the sequence of SEQ ID NOs: 97, 167, and 178, respectively. In one particular embodiment, the anti-PAC1 antibody or antigen-binding fragment thereof comprises a light chain variable region comprising a CDRL1, a CDRL2, and a CDRL3 and a heavy chain variable region comprising a CDRH1, a CDRH2, and a CDRH3, wherein CDRL1, CDRL2, and CDRL3 have the sequence of SEQ ID NOs: 17, 31, and 44, respectively, and CDRH1, CDRH2, and CDRH3 have the sequence of SEQ ID NOs: 98, 168, and 189, respectively. In another particular embodiment, the anti-PAC1 antibody or antigen-binding fragment thereof comprises a light chain variable region comprising a CDRL1, a CDRL2, and a CDRL3 and a heavy chain variable region comprising a CDRH1, a CDRH2, and a CDRH3, wherein CDRL1, CDRL2, and CDRL3 have the sequence of SEQ ID NOs: 17, 27, and 39, respectively, and CDRH1, CDRH2, and CDRH3 have the sequence of SEQ ID NOs: 100, 168, and 182, respectively.

In certain embodiments, the antibodies and antigen-binding fragments of the invention comprise an immunoglobulin heavy chain variable region (VH) and an immunoglobulin light chain variable region (VL) from an antibody that specifically binds to human PAC1, such as the antibodies described herein. The “variable region,” used interchangeably herein with “variable domain” (variable region of a light chain (VL), variable region of a heavy chain (VH)), refers to the region in each of the light and heavy immunoglobulin chains which is involved directly in binding the antibody to the antigen. As discussed above, the regions of variable light and heavy chains have the same general structure and each region comprises four framework (FR) regions, the sequences of which are widely conserved, connected by three CDRs. The framework regions adopt a beta-sheet conformation and the CDRs may form loops connecting the beta-sheet structure. The CDRs in each chain are held in their three-dimensional structure by the framework regions and form, together with the CDRs from the other chain, the antigen binding site.

Thus, in some embodiments, the anti-PAC1 antibodies and antigen-binding fragments of the invention may comprise a light chain variable region selected from LV-01 to LV-15, as shown in Table 1A, and/or a heavy chain variable region selected from HV-01 to HV-105, as shown in Table 1B, and binding fragments, derivatives, and variants of these light chain and heavy chain variable regions. In other embodiments, the anti-PAC1 antibodies and antigen-binding fragments of the invention may comprise a light chain variable region selected from LV-16 to LV-36, as shown in Table 1A, and/or a heavy chain variable region selected from HV-106 to HV-122, as shown in Table 1B, and binding fragments, derivatives, and variants of these light chain and heavy chain variable regions.

Each of the light chain variable regions listed in Table 1A may be combined with any of the heavy chain variable regions listed in Table 1B to form an anti-PAC1 antibody or antigen-binding fragment thereof of the invention. Examples of such combinations include, but are not limited to: (i) LV-03 and any one of HV-03 to HV-26, HV-32 to HV-47, and HV-57 to HV-62; (ii) LV-04 and any one of HV-11 to HV-91; (iii) LV-05 and any one of HV-01, HV-03, HV-07, HV-09, and HV-10; (iv) LV-06 and any one of HV-01, HV-03, HV-07, HV-09, and HV-10; (v) LV-07 and any one of HV-01, HV-03, HV-07, HV-09, and HV-10; (vi) LV-08 and HV-01; (vii) LV-09 and HV-01; (viii) LV-10 and HV-01; (ix) LV-11 and any one of HV-92, HV-93, HV-95 to HV-97, and HV-99 to HV-101; (x) LV-12 and HV-94; (xi) LV-13 and HV-98; (xii) LV-14 and any one of HV-102 to HV-104; (xiii) LV-15 and HV-105; (xiv) LV-17 and HV-107; (xv) LV-18 and HV-108; (xvi) LV-19 and HV-109; (xvii) LV-20 and HV-110; (xviii) LV-21 and HV-110; (xix) LV-22 and HV-111; (xx) LV-23 and HV-107; (xxi) LV-24 and HV-112; (xxii) LV-25 and HV-113; (xxiii) LV-26 and HV-114; (xxiv) LV-27 and HV-106 or HV-110; (xxv) LV-28 and HV-115 or HV-118; (xxvi) LV-29 and HV-116; (xxvii) LV-30 and HV-117; (xxviii) LV-31 and HV-119; (xxix) LV-32 and HV-120; (xxx) LV-33 and HV-121; (xxxi) LV-34 and HV-122; (xxxii) LV-35 and HV-110; and (xxxiii) LV-36 and HV-110.

In certain embodiments, the anti-PACT antibodies or antigen-binding fragments of the invention comprise a light chain variable region comprising the sequence of LV-03 (SEQ ID NO: 54) and a heavy chain variable region comprising the sequence of HV-04 (SEQ ID NO: 194). In some embodiments, the anti-PACT antibodies or antigen-binding fragments of the invention comprise a light chain variable region comprising the sequence of LV-03 (SEQ ID NO: 54) and a heavy chain variable region comprising the sequence of HV-13 (SEQ ID NO: 203). In other embodiments, the anti-PACT antibodies or antigen-binding fragments of the invention comprise a light chain variable region comprising the sequence of LV-04 (SEQ ID NO: 55) and a heavy chain variable region comprising the sequence of HV-38 (SEQ ID NO: 228). In still other embodiments, the anti-PACT antibodies or antigen-binding fragments of the invention comprise a light chain variable region comprising the sequence of LV-04 (SEQ ID NO: 55) and a heavy chain variable region comprising the sequence of HV-84 (SEQ ID NO: 274). In some embodiments, the anti-PACT antibodies or antigen-binding fragments of the invention comprise a light chain variable region comprising the sequence of LV-03 (SEQ ID NO: 54) and a heavy chain variable region comprising the sequence of HV-24 (SEQ ID NO: 214). In certain embodiments, the anti-PACT antibodies or antigen-binding fragments of the invention comprise a light chain variable region comprising the sequence of LV-04 (SEQ ID NO: 55) and a heavy chain variable region comprising the sequence of HV-50 (SEQ ID NO: 240). In one embodiment, the anti-PACT antibodies or antigen-binding fragments of the invention comprise a light chain variable region comprising the sequence of LV-03 (SEQ ID NO: 54) and a heavy chain variable region comprising the sequence of HV-38 (SEQ ID NO: 228). In another embodiment, the anti-PAC1 antibodies or antigen-binding fragments of the invention comprise a light chain variable region comprising the sequence of LV-11 (SEQ ID NO: 62) and a heavy chain variable region comprising the sequence of HV-92 (SEQ ID NO: 282). In yet another embodiment, the anti-PAC1 antibodies or antigen-binding fragments of the invention comprise a light chain variable region comprising the sequence of LV-11 (SEQ ID NO: 62) and a heavy chain variable region comprising the sequence of HV-93 (SEQ ID NO: 283). In still another embodiment, the anti-PAC1 antibodies or antigen-binding fragments of the invention comprise a light chain variable region comprising the sequence of LV-12 (SEQ ID NO: 63) and a heavy chain variable region comprising the sequence of HV-94 (SEQ ID NO: 284).

In certain other embodiments, the anti-PAC1 antibodies or antigen-binding fragments of the invention comprise a light chain variable region comprising the sequence of LV-34 (SEQ ID NO: 85) and a heavy chain variable region comprising the sequence of HV-122 (SEQ ID NO: 312). In some embodiments, the anti-PAC1 antibodies or antigen-binding fragments of the invention comprise a light chain variable region comprising the sequence of LV-21 (SEQ ID NO: 72) and a heavy chain variable region comprising the sequence of HV-110 (SEQ ID NO: 300). In other embodiments, the anti-PAC1 antibodies or antigen-binding fragments of the invention comprise a light chain variable region comprising the sequence of LV-30 (SEQ ID NO: 81) and a heavy chain variable region comprising the sequence of HV-117 (SEQ ID NO: 307). In still other embodiments, the anti-PAC1 antibodies or antigen-binding fragments of the invention comprise a light chain variable region comprising the sequence of LV-27 (SEQ ID NO: 78) and a heavy chain variable region comprising the sequence of HV-106 (SEQ ID NO: 296). In some embodiments, the anti-PAC1 antibodies or antigen-binding fragments of the invention comprise a light chain variable region comprising the sequence of LV-32 (SEQ ID NO: 83) and a heavy chain variable region comprising the sequence of HV-120 (SEQ ID NO: 310). In certain embodiments, the anti-PAC1 antibodies or antigen-binding fragments of the invention comprise a light chain variable region comprising the sequence of LV-36 (SEQ ID NO: 87) and a heavy chain variable region comprising the sequence of HV-110 (SEQ ID NO: 300). In one embodiment, the anti-PAC1 antibodies or antigen-binding fragments of the invention comprise a light chain variable region comprising the sequence of LV-23 (SEQ ID NO: 74) and a heavy chain variable region comprising the sequence of HV-107 (SEQ ID NO: 297). In another embodiment, the anti-PAC1 antibodies or antigen-binding fragments of the invention comprise a light chain variable region comprising the sequence of LV-17 (SEQ ID NO: 68) and a heavy chain variable region comprising the sequence of HV-107 (SEQ ID NO: 297). In yet another embodiment, the anti-PAC1 antibodies or antigen-binding fragments of the invention comprise a light chain variable region comprising the sequence of LV-20 (SEQ ID NO: 71) and a heavy chain variable region comprising the sequence of HV-110 (SEQ ID NO: 300). In still another embodiment, the anti-PAC1 antibodies or antigen-binding fragments of the invention comprise a light chain variable region comprising the sequence of LV-26 (SEQ ID NO: 77) and a heavy chain variable region comprising the sequence of HV-114 (SEQ ID NO: 304).

In some embodiments, the anti-PAC1 antibodies or antigen-binding fragments thereof comprise a light chain variable region comprising a sequence of contiguous amino acids that differs from the sequence of a light chain variable region in Table 1A, i.e. a VL selected from LV-01 to LV-36, at only 1, 2, 3, 4, 5, 6, 7, 8, 9, 10, 11, 12, 13, 14 or 15 amino acid residues, wherein each such sequence difference is independently either a deletion, insertion or substitution of one amino acid, with the deletions, insertions and/or substitutions resulting in no more than 15 amino acid changes relative to the foregoing variable domain sequences. The light chain variable region in some anti-PAC1 antibodies and binding fragments comprises a sequence of amino acids that has at least 70%, at least 75%, at least 80%, at least 85%, at least 90%, at least 95%, at least 97% or at least 99% sequence identity to the amino acid sequences of SEQ ID NOs: 52 to 87 (i.e. the light chain variable regions in Table 1A).

In one embodiment, the anti-PAC1 antibody or antigen-binding fragment thereof comprises a light chain variable region comprising a sequence that is at least 90% identical to a sequence selected from SEQ ID NOs: 54-66. In another embodiment, the anti-PAC1 antibody or antigen-binding fragment thereof comprises a light chain variable region comprising a sequence that is at least 95% identical to a sequence selected from SEQ ID NOs: 54-66. In yet another embodiment, the anti-PAC1 antibody or antigen-binding fragment thereof comprises a light chain variable region comprising a sequence selected from SEQ ID NOs: 54-66. In some embodiments, the anti-PAC1 antibody or antigen-binding fragment thereof comprises a light chain variable region comprising the sequence of SEQ ID NO: 54. In other embodiments, the anti-PAC1 antibody or antigen-binding fragment thereof comprises a light chain variable region comprising the sequence of SEQ ID NO: 55. In yet other embodiments, the anti-PAC1 antibody or antigen-binding fragment thereof comprises a light chain variable region comprising the sequence of SEQ ID NO: 62. In still other embodiments, the anti-PAC1 antibody or antigen-binding fragment thereof comprises a light chain variable region comprising the sequence of SEQ ID NO: 63.

In another embodiment, the anti-PAC1 antibody or antigen-binding fragment thereof comprises a light chain variable region comprising a sequence that is at least 90% identical to a sequence selected from SEQ ID NOs: 68-87. In yet another embodiment, the anti-PAC1 antibody or antigen-binding fragment thereof comprises a light chain variable region comprising a sequence that is at least 95% identical to a sequence selected from SEQ ID NOs: 68-87. In still another embodiment, the anti-PAC1 antibody or antigen-binding fragment thereof comprises a light chain variable region comprising a sequence selected from SEQ ID NOs: 68-87. In certain embodiments, the anti-PAC1 antibody or antigen-binding fragment thereof comprises a light chain variable region comprising the sequence of SEQ ID NO: 68. In some embodiments, the anti-PAC1 antibody or antigen-binding fragment thereof comprises a light chain variable region comprising the sequence of SEQ ID NO: 71. In other embodiments, the anti-PAC1 antibody or antigen-binding fragment thereof comprises a light chain variable region comprising the sequence of SEQ ID NO: 72. In yet other embodiments, the anti-PAC1 antibody or antigen-binding fragment thereof comprises a light chain variable region comprising the sequence of SEQ ID NO: 74. In still other embodiments, the anti-PAC1 antibody or antigen-binding fragment thereof comprises a light chain variable region comprising the sequence of SEQ ID NO: 77. In certain embodiments, the anti-PAC1 antibody or antigen-binding fragment thereof comprises a light chain variable region comprising the sequence of SEQ ID NO: 78. In other embodiments, the anti-PAC1 antibody or antigen-binding fragment thereof comprises a light chain variable region comprising the sequence of SEQ ID NO: 81. In one embodiment, the anti-PAC1 antibody or antigen-binding fragment thereof comprises a light chain variable region comprising the sequence of SEQ ID NO: 83. In another embodiment, the anti-PAC1 antibody or antigen-binding fragment thereof comprises a light chain variable region comprising the sequence of SEQ ID NO: 85. In yet another embodiment, the anti-PAC1 antibody or antigen-binding fragment thereof comprises a light chain variable region comprising the sequence of SEQ ID NO: 87.

In these and other embodiments, the anti-PAC1 antibodies and antigen-binding fragments thereof comprise a heavy chain variable region comprising a sequence of contiguous amino acids that differs from the sequence of a heavy chain variable region in Table 1B, i.e., a VH selected from HV-01 to HV-122, at only 1, 2, 3, 4, 5, 6, 7, 8, 9, 10, 11, 12, 13, 14 or 15 amino acid residues, wherein each such sequence difference is independently either a deletion, insertion or substitution of one amino acid, with the deletions, insertions and/or substitutions resulting in no more than 15 amino acid changes relative to the foregoing variable domain sequences. The heavy chain variable region in some anti-PAC1 antibodies and antigen-binding fragments comprises a sequence of amino acids that has at least 70%, at least 75%, at least 80%, at least 85%, at least 90%, at least 95%, at least 97% or at least 99% sequence identity to the amino acid sequences of SEQ ID NOs: 191 to 312 (i.e. the heavy chain variable regions in Table 1B).

In one embodiment, the anti-PAC1 antibody or antigen-binding fragment thereof comprises a heavy chain variable region comprising a sequence that is at least 90% identical to a sequence selected from SEQ ID NOs: 191-295. In another embodiment, the anti-PAC1 antibody or antigen-binding fragment thereof comprises a heavy chain variable region comprising a sequence that is at least 95% identical to a sequence selected from SEQ ID NOs: 191-295. In yet another embodiment, the anti-PAC1 antibody or antigen-binding fragment thereof comprises a heavy chain variable region comprising a sequence selected from SEQ ID NOs: 191-295. In some embodiments, the anti-PAC1 antibody or antigen-binding fragment thereof comprises a heavy chain variable region comprising the sequence of SEQ ID NO: 194. In other embodiments, the anti-PAC1 antibody or antigen-binding fragment thereof comprises a heavy chain variable region comprising the sequence of SEQ ID NO: 203. In yet other embodiments, the anti-PAC1 antibody or antigen-binding fragment thereof comprises a heavy chain variable region comprising the sequence of SEQ ID NO: 214. In still other embodiments, the anti-PAC1 antibody or antigen-binding fragment thereof comprises a heavy chain variable region comprising the sequence of SEQ ID NO: 228. In certain embodiments, the anti-PAC1 antibody or antigen-binding fragment thereof comprises a heavy chain variable region comprising the sequence of SEQ ID NO: 240. In other embodiments, the anti-PAC1 antibody or antigen-binding fragment thereof comprises a heavy chain variable region comprising the sequence of SEQ ID NO: 274. In one embodiment, the anti-PAC1 antibody or antigen-binding fragment thereof comprises a heavy chain variable region comprising the sequence of SEQ ID NO: 282. In another embodiment, the anti-PAC1 antibody or antigen-binding fragment thereof comprises a heavy chain variable region comprising the sequence of SEQ ID NO: 283. In yet another embodiment, the anti-PAC1 antibody or antigen-binding fragment thereof comprises a heavy chain variable region comprising the sequence of SEQ ID NO: 284.

In another embodiment, the anti-PAC1 antibody or antigen-binding fragment thereof comprises a heavy chain variable region comprising a sequence that is at least 90% identical to a sequence selected from SEQ ID NOs: 296-312. In yet another embodiment, the anti-PAC1 antibody or antigen-binding fragment thereof comprises a heavy chain variable region comprising a sequence that is at least 95% identical to a sequence selected from SEQ ID NOs: 296-312. In still another embodiment, the anti-PAC1 antibody or antigen-binding fragment thereof comprises a heavy chain variable region comprising a sequence selected from SEQ ID NOs: 296-312. In some embodiments, the anti-PAC1 antibody or antigen-binding fragment thereof comprises a heavy chain variable region comprising the sequence of SEQ ID NO: 296. In other embodiments, the anti-PAC1 antibody or antigen-binding fragment thereof comprises a heavy chain variable region comprising the sequence of SEQ ID NO: 297. In yet other embodiments, the anti-PAC1 antibody or antigen-binding fragment thereof comprises a heavy chain variable region comprising the sequence of SEQ ID NO: 300. In still other embodiments, the anti-PAC1 antibody or antigen-binding fragment thereof comprises a heavy chain variable region comprising the sequence of SEQ ID NO: 304. In certain embodiments, the anti-PAC1 antibody or antigen-binding fragment thereof comprises a heavy chain variable region comprising the sequence of SEQ ID NO: 307. In other embodiments, the anti-PAC1 antibody or antigen-binding fragment thereof comprises a heavy chain variable region comprising the sequence of SEQ ID NO: 310. In one embodiment, the anti-PAC1 antibody or antigen-binding fragment thereof comprises a heavy chain variable region comprising the sequence of SEQ ID NO: 312.

The term “identity,” as used herein, refers to a relationship between the sequences of two or more polypeptide molecules or two or more nucleic acid molecules, as determined by aligning and comparing the sequences. “Percent identity,” as used herein, means the percent of identical residues between the amino acids or nucleotides in the compared molecules and is calculated based on the size of the smallest of the molecules being compared. For these calculations, gaps in alignments (if any) must be addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al., 1988, SIAM J. Applied Math. 48:1073. For example, sequence identity can be determined by standard methods that are commonly used to compare the similarity in position of the amino acids of two polypeptides. Using a computer program such as BLAST or FASTA, two polypeptide or two polynucleotide sequences are aligned for optimal matching of their respective residues (either along the full length of one or both sequences, or along a pre-determined portion of one or both sequences). The programs provide a default opening penalty and a default gap penalty, and a scoring matrix such as PAM 250 (Dayhoff et al., in Atlas of Protein Sequence and Structure, vol. 5, supp. 3, 1978) or BLOSUM62 (Henikoff et al., 1992, Proc. Natl. Acad. Sci. U.S.A. 89:10915-10919) can be used in conjunction with the computer program. For example, the percent identity can then be calculated as: the total number of identical matches multiplied by 100 and then divided by the sum of the length of the longer sequence within the matched span and the number of gaps introduced into the longer sequences in order to align the two sequences. In calculating percent identity, the sequences being compared are aligned in a way that gives the largest match between the sequences.

The GCG program package is a computer program that can be used to determine percent identity, which package includes GAP (Devereux et al., 1984, Nucl. Acid Res. 12:387; Genetics Computer Group, University of Wisconsin, Madison, WI). The computer algorithm GAP is used to align the two polypeptides or two polynucleotides for which the percent sequence identity is to be determined. The sequences are aligned for optimal matching of their respective amino acid or nucleotide (the “matched span,” as determined by the algorithm). A gap opening penalty (which is calculated as 3× the average diagonal, wherein the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. In certain embodiments, a standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. U.S.A. 89:10915-10919 for the BLOSUM 62 comparison matrix) is also used by the algorithm.

Recommended parameters for determining percent identity for polypeptides or nucleotide sequences using the GAP program include the following:
Algorithm: Needleman et al. 1970, J. Mol. Biol. 48:443-453;
Comparison matrix: BLOSUM 62 from Henikoff et al., 1992, supra;
Gap Penalty: 12 (but with no penalty for end gaps)
Gap Length Penalty: 4
Threshold of Similarity: 0

Certain alignment schemes for aligning two amino acid sequences may result in matching of only a short region of the two sequences, and this small aligned region may have very high sequence identity even though there is no significant relationship between the two full-length sequences. Accordingly, the selected alignment method (GAP program) can be adjusted if so desired to result in an alignment that spans at least 50 contiguous amino acids of the target polypeptide.

The anti-PAC1 antibodies of the invention can comprise any immunoglobulin constant region. The term “constant region,” used interchangeably herein with “constant domain” refers to all domains of an antibody other than the variable region. The constant region is not involved directly in binding of an antigen, but exhibits various effector functions. As described above, antibodies are divided into particular isotypes (IgA, IgD, IgE, IgG, and IgM) and subtypes (IgG1, IgG2, IgG3, IgG4, IgA1 IgA2) depending on the amino acid sequence of the constant region of their heavy chains. The light chain constant region can be, for example, a kappa- or lambda-type light chain constant region, e.g., a human kappa- or lambda-type light chain constant region, which are found in all five antibody isotypes. Examples of human immunoglobulin light chain constant region sequences are shown in the following table.

TABLE 2
Exemplary Human Immunoglobulin Light Chain Constant Regions

| | SEQ ID | |
|---|---|---|
| Designation | NO: | CL Domain Amino Acid Sequence |
| Human | 313 | GQPKANPTVTLFPPSSEELQANKATLVCLISDFYPGAVTVAWKA |
| lambda v1 | | DGSPVKAGVETTKPSKQSNNKYAASSYLSLTPEQWKSHRSYSC |
| | | QVTHEGSTVEKTVAPTECS |
| Human | 314 | GQPKAAPSVTLFPPSSEELQANKATLVCLISDFYPGAVTVAWKA |
| lambda v2 | | DSSPVKAGVETTTPSKQSNNKYAASSYLSLTPEQWKSHRSYSCQ |
| | | VTHEGSTVEKTVAPTECS |
| Human | 315 | QPKAAPSVTLFPPSSEELQANKATLVCLISDFYPGAVTVAWKAD |
| lambda v3 | | SSPVKAGVETTTPSKQSNNKYAASSYLSLTPEQWKSHRSYSCQV |
| | | THEGSTVEKTVAPTECS |
| Human | 316 | GQPKAAPSVTLFPPSSEELQANKATLVCLISDFYPGAVTVAWKA |
| lambda v4 | | DSSPVKAGVETTTPSKQSNNKYAASSYLSLTPEQWKSHKSYSCQ |
| | | VTHEGSTVEKTVAPTECS |
| Human | 317 | GQPKAAPSVTLFPPSSEELQANKATLVCLVSDFYPGAVTVAWK |
| lambda v5 | | ADGSPVKVGVETTKPSKQSNNKYAASSYLSLTPEQWKSHRSYS |
| | | CRVTHEGSTVEKTVAPAECS |
| Human | 318 | TVAAPSVFIFPPSDEQLKSGTASVVCLLNNFYPREAKVQWKVDN |
| kappa v1 | | ALQSGNSQESVTEQDSKDSTYSLSSTLTLSKADYEKHKVYACEV |
| | | THQGLSSPVTKSFNRGEC |
| Human | 319 | RTVAAPSVFIFPPSDEQLKSGTASVVCLLNNFYPREAKVQWKVD |
| kappa v2 | | NALQSGNSQESVTEQDSKDSTYSLSSTLTLSKADYEKHKVYACE |
| | | VTHQGLSSPVTKSFNRGEC |

The heavy chain constant region of the anti-PAC1 antibodies of the invention can be, for example, an alpha-, delta-, epsilon-, gamma-, or mu-type heavy chain constant region, e.g., a human alpha-, delta-, epsilon-, gamma-, or mu-type heavy chain constant region. In some embodiments, the anti-PAC1 antibodies comprise a heavy chain constant region from an IgG1, IgG2, IgG3, or IgG4 immunoglobulin, such as a human IgG1, IgG2, IgG3, or IgG4 immunoglobulin. In one embodiment, the anti-PAC1 antibody comprises a heavy chain constant region from a human IgG1 immunoglobulin. In such embodiments, the human IgG1 immunoglobulin constant region may comprise one or more mutations to prevent glycosylation of the antibody as described in more detail herein. In another embodiment, the anti-PAC1 antibody comprises a heavy chain constant region from a human IgG2 immunoglobulin. In yet another embodiment, the anti-PAC1 antibody comprises a heavy chain constant region from a human IgG4 immunoglobulin. Examples of human IgG1, IgG2, and IgG4 heavy chain constant region sequences are shown below in Table 3.

TABLE 3
Exemplary Human Immunoglobulin Heavy Chain Constant Regions

| | SEQ ID | |
|---|---|---|
| Ig isotype | NO: | Heavy Chain Constant Region Amino Acid Sequence |
| Human IgG1z | 320 | ASTKGPSVFPLAPSSKSTSGGTAALGCLVKDYFPEPVTVSWN |
| | | SGALTSGVHTFPAVLQSSGLYSLSSVVTVPSSSLGTQTYICNV |
| | | NHKPSNTKVDKKVEPKSCDKTHTCPPCPAPELLGGPSVFLFP |
| | | PKPKDTLMISRTPEVTCVVVDVSHEDPEVKFNWYVDGVEVH |
| | | NAKTKPREEQYNSTYRVVSVLTVLHQDWLNGKEYKCKVSN |
| | | KALPAPIEKTISKAKGQPREPQVYTLPPSREEMTKNQVSLTCL |
| | | VKGFYPSDIAVEWESNGQPENNYKTTPPVLDSDGSFFLYSKL |
| | | TVDKSRWQQGNVFSCSVMHEALHNHYTQKSLSLSPGK |
| Human IgG1za | 321 | ASTKGPSVFPLAPSSKSTSGGTAALGCLVKDYFPEPVTVSWN |
| | | SGALTSGVHTFPAVLQSSGLYSLSSVVTVPSSSLGTQTYICNV |
| | | NHKPSNTKVDKKVEPKSCDKTHTCPPCPAPELLGGPSVFLFP |
| | | PKPKDTLMISRTPEVTCVVVDVSHEDPEVKFNWYVDGVEVH |
| | | NAKTKPREEQYNSTYRVVSVLTVLHQDWLNGKEYKCKVSN |
| | | KALPAPIEKTISKAKGQPREPQVYTLPPSRDELTKNQVSLTCL |
| | | VKGFYPSDIAVEWESNGQPENNYKTTPPVLDSDGSFFLYSKL |
| | | TVDKSRWQQGNVFSCSVMHEALHNHYTQKSLSLSPGK |
| Human IgG1f | 322 | ASTKGPSVFPLAPSSKSTSGGTAALGCLVKDYFPEPVTVSWN |
| | | SGALTSGVHTFPAVLQSSGLYSLSSVVTVPSSSLGTQTYICNV |
| | | NHKPSNTKVDKRVEPKSCDKTHTCPPCPAPELLGGPSVFLFP |
| | | PKPKDTLMISRTPEVTCVVVDVSHEDPEVKFNWYVDGVEVH |
| | | NAKTKPREEQYNSTYRVVSVLTVLHQDWLNGKEYKCKVSN |
| | | KALPAPIEKTISKAKGQPREPQVYTLPPSREEMTKNQVSLTCL |
| | | VKGFYPSDIAVEWESNGQPENNYKTTPPVLDSDGSFFLYSKL |
| | | TVDKSRWQQGNVFSCSVMHEALHNHYTQKSLSLSPGK |
| Human IgG1fa | 323 | ASTKGPSVFPLAPSSKSTSGGTAALGCLVKDYFPEPVTVSWN |
| | | SGALTSGVHTFPAVLQSSGLYSLSSVVTVPSSSLGTQTYICNV |
| | | NHKPSNTKVDKRVEPKSCDKTHTCPPCPAPELLGGPSVFLFP |
| | | PKPKDTLMISRTPEVTCVVVDVSHEDPEVKFNWYVDGVEVH |
| | | NAKTKPREEQYNSTYRVVSVLTVLHQDWLNGKEYKCKVSN |
| | | KALPAPIEKTISKAKGQPREPQVYTLPPSRDELTKNQVSLTCL |
| | | VKGFYPSDIAVEWESNGQPENNYKTTPPVLDSDGSFFLYSKL |
| | | TVDKSRWQQGNVFSCSVMHEALHNHYTQKSLSLSPGK |
| Human IgG1z | 324 | ASTKGPSVFPLAPSSKSTSGGTAALGCLVKDYFPEPVTVSWN |
| aglycosylated | | SGALTSGVHTFPAVLQSSGLYSLSSVVTVPSSSLGTQTYICNV |
| v1 | | NHKPSNTKVDKKVEPKSCDKTHTCPPCPAPELLGGPSVFLFP |
| | | PKPKDTLMISRTPEVTCVVVDVSHEDPEVKFNWYVDGVEVH |
| | | NAKTKPREEQYGSTYRVVSVLTVLHQDWLNGKEYKCKVSN |
| | | KALPAPIEKTISKAKGQPREPQVYTLPPSREEMTKNQVSLTCL |
| | | VKGFYPSDIAVEWESNGQPENNYKTTPPVLDSDGSFFLYSKL |
| | | TVDKSRWQQGNVFSCSVMHEALHNHYTQKSLSLSPGK |
| Human IgG1z | 325 | ASTKGPSVFPLAPSSKSTSGGTAALGCLVKDYFPEPVTVSWN |
| aglycosylated | | SGALTSGVHTFPAVLQSSGLYSLSSVVTVPSSSLGTQTYICNV |
| v2 | | NHKPSNTKVDKKVEPKSCDKTHTCPPCPAPELLGGPSVFLFP |
| | | PKPKDTLMISRTPEVTCVVVDVSHEDPEVKFNWYVDGVEVH |
| | | NAKTKPCEEQYGSTYRCVSVLTVLHQDWLNGKEYKCKVSN |
| | | KALPAPIEKTISKAKGQPREPQVYTLPPSREEMTKNQVSLTCL |
| | | VKGFYPSDIAVEWESNGQPENNYKTTPPVLDSDGSFFLYSKL |
| | | TVDKSRWQQGNVFSCSVMHEALHNHYTQKSLSLSPGK |
| Human IgG2 | 326 | ASTKGPSVFPLAPCSRSTSESTAALGCLVKDYFPEPVTVSWN |
| | | SGALTSGVHTFPAVLQSSGLYSLSSVVTVPSSNFGTQTYTCN |
| | | VDHKPSNTKVDKTVERKCCVECPPCPAPPVAGPSVFLFPPKP |
| | | KDTLMISRTPEVTCVVVDVSHEDPEVQFNWYVDGVEVHNA |
| | | KTKPREEQFNSTFRVVSVLTVVHQDWLNGKEYKCKVSNKG |
| | | LPAPIEKTISKTKGQPREPQVYTLPPSREEMTKNQVSLTCLVK |
| | | GFYPSDIAVEWESNGQPENNYKTTPPMLDSDGSFFLYSKLTV |
| | | DKSRWQQGNVFSCSVMHEALHNHYTQKSLSLSPGK |
| Human IgG4 | 327 | ASTKGPSVFPLAPSSKSTSGGTAALGCLVKDYFPEPVTVSWN |
| | | SGALTSGVHTFPAVLQSSGLYSLSSVVTVPSSSLGTQTYICNV |
| | | NHKPSNTKVDKKVEPKSCDKTHTCPPCPAPELLGGPSVFLFP |
| | | PKPKDTLMISRTPEVTCVVVDVSHEDPEVKFNWYVDGVEVH |
| | | NAKTKPCEEQYGSTYRCVSVLTVLHQDWLNGKEYKCKVSN |
| | | KALPAPIEKTISKAKGQPREPQVYTLPPSREEMTKNQVSLTCL |
| | | VKGFYPSDIAVEWESNGQPENNYKTTPPVLDSDGSFFLYSKL |
| | | TVDKSRWQQGNVFSCSVMHEALHNHYTQKSLSLSPGK |

Each of the light chain variable regions disclosed in Table 1A and each of the heavy chain variable regions disclosed in Table 1B may be attached to the above light chain constant regions (Table 2) and heavy chain constant regions (Table 3) to form complete antibody light and heavy chains, respectively. Further, each of the so generated heavy and light chain sequences may be combined to form a complete antibody structure. It should be understood that the heavy chain and light chain variable regions provided herein can also be attached to other constant domains having different sequences than the exemplary sequences listed above.

The anti-PAC1 antibodies or antigen-binding fragments of the invention can be any of the anti-PAC1 antibodies or antigen-binding fragments disclosed herein. For example, in certain embodiments, the anti-PAC1 antibody is an anti-PAC1 antibody selected from any of the antibodies listed in Tables 9, 10, 12, 13, 14, 19, and 20. In some embodiments, the anti-PAC1 antibodies comprise a light chain variable region and/or a heavy chain variable region having one or more of the amino acid substitutions set forth in Tables 9, 10, 12, 13, 19, or 20. For instance, in one embodiment, the anti-PAC1 antibody comprises a light chain variable region comprising the sequence of SEQ ID NO: 52 with a mutation at one or more amino acid positions 27, 28, 30, 31, 32, and/or 93. In certain embodiments, the mutation is selected from Q27K, Q27R, Q27H, S28A, G30M, G30W, R31Q, R31L, R31H, R31W, R31Y, S32A, S32N, S32L, R93F, R93M, R93Y, or combinations thereof. In these and other embodiments, the anti-PAC1 antibody comprises a heavy chain variable region comprising the sequence of SEQ ID NO: 191 with a mutation at one or more amino acid positions 31, 32, 49, 52, 53, 54, 56, 57, 62, 70, 102, 103, and/or 104. In some embodiments, the mutation is selected from R31F, R31H, R31Y, R31M, R31K, F32Y, A49G, S52N, S52T, Y53F, Y53H, Y53S, D541, D54L, D54N, D54R, D54Q, D54Y, D54F, D54M, D54S, D54T, G56Q, G56N, G56R, G56H, G56A, G56S, G56T, G56D, N57A, N57F, N57G, N57S, N57Q, N57L, N57T, E62R, I70V, I70L, I70M, V102P, V102L, V102I, V102F, V102M, L103M, T104S, or combinations thereof.

In another embodiment, the anti-PAC1 antibody comprises a light chain variable region comprising the sequence of SEQ ID NO: 67 with a mutation at one or more amino acid positions 28, 30, 31, 34, 49, 50, 51, 52, 53, 91, 92, 93, 94, and/or 96. The mutation can be selected from S28Y, S28T, S28K, S28P, S28Q, S28R, S28M, S30A, S30V, R31Q, N34V, N34S, Y49F, A50V, A50G, A51G, A51S, S52Q, S52H, S52N, S52Y, S52R, S52L, S53I, S53Y, S53N, S53M, S53H, S53R, S91A, Y92I, S93G, S93Q, S93I, S93N, S93M, P94E, P94M, P94N, P94Q, P94A, P94T, P941, F96Y, or combinations thereof. In these and other embodiments, the anti-PAC1 antibody comprises a heavy chain variable region comprising the sequence of SEQ ID NO: 296 with a mutation at one or more amino acid positions 31, 32, 33, 57, 58, 60, 103, 105, 106, 107, and/or 110. In some embodiments, the mutation is selected from S31N, N32R, N32K, N32H, S33L, S33Q, S33Y, S33V, S33D, S57G, K58Q, S60K, T103M, T103Q, T103R, T103V, K105N, K105E, K105D, K1051, K105A, K1055, K105Q, Q106G, Q106E, L107D, L107N, L110M, L110F, or combinations thereof.

In certain embodiments, the anti-PAC1 antibody or antigen-binding fragment of the invention is selected from antibodies 420653, 420845, 420943, 421873, 420889, 421091, 421051, 480711, 480706, 480713, 448730, 448195, 448788, 448901, 448689, 448202, 452128, 448924, 3574, and 3575 or antigen-binding fragments thereof, the variable region and CDR sequences of which are set forth in Tables 1A and 1B. In some embodiments, the anti-PAC1 antibody is an antibody selected from 420653, 420845, 420943, 421873, 420889, 421091, 421051, 480711, 480706, and 480713 antibodies. In other embodiments, the anti-PAC1 antibody is an antibody selected from 448730, 448195, 448788, 448901, 448689, 448202, 452128, 448924, 3574, and 3575 antibodies. Full-length light chain and full-length heavy chain sequences of these exemplary human anti-PAC1 antibodies are set forth below in Tables 4A and 4B, respectively.

TABLE 4A
Exemplary Anti-PAC1 Antibody Light Chain Sequences

| | | | |
|---|---|---|---|
| Antibody | LC | Light Chain | Light Chain |
| ID. | Group | Amino Acid Sequence | Nucleic Acid Sequence |

29G4 variants

| | | | |
|---|---|---|---|
| 29G4v10 | LC-01 | EIVLTQSPATLSLSPGERATLSCRA | GAGATCGTACTTACTCAGTCACCCGCCACA |
| | | SQSVGRSLHWYQQKPGQAPRLLI | TTGTCCCTGAGCCCGGGTGAACGGGCGACC |
| | | KYASQSLSGIPARFSGSGSGTDFT | CTCAGCTGCCGAGCATCCCAGTCCGTCGGA |
| | | LTISSLEPEDFAVYYCHQSSRLPFT | CGATCATTGCACTGGTACCAACAAAAACCG |
| | | FGPGTKVDIKRTVAAPSVFIFPPSD | GGCCAGGCCCCCAGACTTCTGATCAAGTAT |
| | | EQLKSGTASVVCLLNNFYPREAK | GCGTCACAGAGCTTGTCGGGTATTCCCGCTC |
| | | VQWKVDNALQSGNSQESVTEQD | GCTTTTCGGGGTCGGGATCCGGGACAGATT |
| | | SKDSTYSLSSTLTLSKADYEKHKV | TCACGCTCACAATCTCCTCGCTGGAACCCG |
| | | YACEVTHQGLSSPVTKSFNRGEC | AGGACTTCGCGGTCTACTATTGTCATCAGTC |
| | | (SEQ ID NO: 504) | ATCGAGGTTGCCTTTCACGTTTGGACCAGG |
| | | | GACCAAGGTGGACATTAAGCGTACGGTGGC |
| | | | TGCACCATCTGTCTTCATCTTCCCGCCATCT |
| | | | GATGAGCAGTTGAAATCTGGAACTGCCTCT |
| | | | GTTGTGTGCCTGCTGAATAACTTCTATCCCA |
| | | | GAGAGGCCAAAGTACAGTGGAAGGTGGAT |
| | | | AACGCCCTCCAATCGGGTAACTCCCAGGAG |
| | | | AGTGTCACAGAGCAGGACAGCAAGGACAG |
| | | | CACCTACAGCCTCAGCAGCACCCTGACGCT |
| | | | GAGCAAAGCAGACTACGAGAAACACAAAG |
| | | | TCTACGCCTGCGAAGTCACCCATCAGGGCC |
| | | | TGAGCTCGCCCGTCACAAAGAGCTTCAACA |
| | | | GGGGAGAGTGTTAG (SEQ ID NO: 537) |
| 29G4v22 | LC-02 | DIQLTQSPSFLSASVGDRVTITCRA | GATATCCAGCTCACTCAATCGCCATCATTTC |
| | | SQSIGRSLHWYQQKPGKAPKLLIK | TCTCCGCTTCGGTAGGCGACCGGGTCACGA |
| | | YASQSLSGVPSRFSGSGSGTEFTL | TCACATGCAGGGCGTCGCAAAGCATTGGGA |
| | | TISSLQPEDFATYYCHQSSRLPFTF | GGTCGTTGCATTGGTATCAGCAGAAACCCG |
| | | GPGTKVDIKRTVAAPSVFIFPPSDE | GAAAGGCCCCGAAACTTCTGATCAAATACG |
| | | QLKSGTASVVCLLNNFYPREAKV | CATCACAAAGCTTGAGCGGTGTGCCGTCGC |
| | | QWKVDNALQSGNSQESVTEQDS | GCTTCTCCGGTTCCGGAAGCGGAACGGAAT |
| | | KDSTYSLSSTLTLSKADYEKHKV | TCACGCTTACAATCTCCTCACTGCAGCCCGA |
| | | YACEVTHQGLSSPVTKSFNRGEC | GGATTTCGCGACCTATTACTGTCACCAGTCA |
| | | (SEQ ID NO: 505) | TCCAGACTCCCGTTTACTTTTGGCCCTGGGA |
| | | | CCAAGGTGGACATTAAGCGTACGGTGGCTG |
| | | | CACCATCTGTCTTCATCTTCCCGCCATCTGA |
| | | | TGAGCAGTTGAAATCTGGAACTGCCTCTGTT |
| | | | GTGTGCCTGCTGAATAACTTCTATCCCAGAG |
| | | | AGGCCAAAGTACAGTGGAAGGTGGATAAC |
| | | | GCCCTCCAATCGGGTAACTCCCAGGAGAGT |
| | | | GTCACAGAGCAGGACAGCAAGGACAGCAC |
| | | | CTACAGCCTCAGCAGCACCCTGACGCTGAG |
| | | | CAAAGCAGACTACGAGAAACACAAAGTCTA |
| | | | CGCCTGCGAAGTCACCCATCAGGGCCTGAG |
| | | | CTCGCCCGTCACAAAGAGCTTCAACAGGGG |
| | | | AGAGTGT (SEQ ID NO: 538) |
| iPS:420653 | LC-03 | EIVLTQSPATLSLSPGERATLSCRA | GAGATCGTACTTACTCAGTCACCCGCCACA |
| | | SKSVGRSLHWYQQKPGQAPRLLI | TTGTCCCTGAGCCCGGGTGAACGGGCGACC |
| | | KYASQSLSGIPARFSGSGSGTDFT | CTCAGCTGCCGAGCATCCAAGTCCGTCGGA |
| | | LTISSLEPEDFAVYYCHQSSRLPFT | CGATCATTGCACTGGTACCAACAAAAACCG |
| | | FGPGTKVDIKRTVAAPSVFIFPPSD | GGCCAGGCCCCCAGACTTCTGATCAAGTAT |
| | | EQLKSGTASVVCLLNNFYPREAK | GCGTCACAGAGCTTGTCGGGTATTCCCGCTC |
| | | VQWKVDNALQSGNSQESVTEQD | GCTTTTCGGGGTCGGGATCCGGGACAGATT |
| | | SKDSTYSLSSTLTLSKADYEKHKV | TCACGCTCACAATCTCCTCGCTGGAACCCG |
| | | YACEVTHQGLSSPVTKSFNRGEC | AGGACTTCGCGGTCTACTATTGTCATCAGTC |
| | | (SEQ ID NO: 506) | ATCGAGGTTGCCTTTCACGTTTGGACCAGG |
| | | | GACCAAGGTGGACATTAAGCGTACGGTGGC |
| | | | TGCACCATCTGTCTTCATCTTCCCGCCATCT |
| | | | GATGAGCAGTTGAAATCTGGAACTGCCTCT |
| | | | GTTGTGTGCCTGCTGAATAACTTCTATCCCA |
| | | | GAGAGGCCAAAGTACAGTGGAAGGTGGAT |
| | | | AACGCCCTCCAATCGGGTAACTCCCAGGAG |
| | | | AGTGTCACAGAGCAGGACAGCAAGGACAG |
| | | | CACCTACAGCCTCAGCAGCACCCTGACGCT |
| | | | GAGCAAAGCAGACTACGAGAAACACAAAG |
| | | | TCTACGCCTGCGAAGTCACCCATCAGGGCC |
| | | | TGAGCTCGCCCGTCACAAAGAGCTTCAACA |
| | | | GGGGAGAGTGT (SEQ ID NO: 539) |
| iPS:420845 | LC-03 | EIVLTQSPATLSLSPGERATLSCRA | GAGATCGTACTTACTCAGTCACCCGCCACA |
| | | SKSVGRSLHWYQQKPGQAPRLLI | TTGTCCCTGAGCCCGGGTGAACGGGCGACC |
| | | KYASQSLSGIPARFSGSGSGTDFT | CTCAGCTGCCGAGCATCCAAGTCCGTCGGA |
| | | LTISSLEPEDFAVYYCHQSSRLPFT | CGATCATTGCACTGGTACCAACAAAAACCG |
| | | FGPGTKVDIKRTVAAPSVFIFPPSD | GGCCAGGCCCCCAGACTTCTGATCAAGTAT |
| | | EQLKSGTASVVCLLNNFYPREAK | GCGTCACAGAGCTTGTCGGGTATTCCCGCTC |
| | | VQWKVDNALQSGNSQESVTEQD | GCTTTTCGGGGTCGGGATCCGGGACAGATT |
| | | SKDSTYSLSSTLTLSKADYEKHKV | TCACGCTCACAATCTCCTCGCTGGAACCCG |
| | | YACEVTHQGLSSPVTKSFNRGEC | AGGACTTCGCGGTCTACTATTGTCATCAGTC |
| | | (SEQ ID NO: 506) | ATCGAGGTTGCCTTTCACGTTTGGACCAGG |
| | | | GACCAAGGTGGACATTAAGCGTACGGTGGC |
| | | | TGCACCATCTGTCTTCATCTTCCCGCCATCT |
| | | | GATGAGCAGTTGAAATCTGGAACTGCCTCT |
| | | | GTTGTGTGCCTGCTGAATAACTTCTATCCCA |
| | | | GAGAGGCCAAAGTACAGTGGAAGGTGGAT |
| | | | AACGCCCTCCAATCGGGTAACTCCCAGGAG |
| | | | AGTGTCACAGAGCAGGACAGCAAGGACAG |
| | | | CACCTACAGCCTCAGCAGCACCCTGACGCT |
| | | | GAGCAAAGCAGACTACGAGAAACACAAAG |
| | | | TCTACGCCTGCGAAGTCACCCATCAGGGCC |
| | | | TGAGCTCGCCCGTCACAAAGAGCTTCAACA |
| | | | GGGGAGAGTGT(SEQ ID NO: 539) |
| iPS:420943 | LC-01 | EIVLTQSPATLSLSPGERATLSCRA | GAGATCGTACTTACTCAGTCACCCGCCACA |
| | | SQSVGRSLHWYQQKPGQAPRLLI | TTGTCCCTGAGCCCGGGTGAACGGGCGACC |
| | | KYASQSLSGIPARFSGSGSGTDFT | CTCAGCTGCCGAGCATCCCAGTCCGTCGGA |
| | | LTISSLEPEDFAVYYCHQSSRLPFT | CGATCATTGCACTGGTACCAACAAAAACCG |
| | | FGPGTKVDIKRTVAAPSVFIFPPSD | GGCCAGGCCCCCAGACTTCTGATCAAGTAT |
| | | EQLKSGTASVVCLLNNFYPREAK | GCGTCACAGAGCTTGTCGGGTATTCCCGCTC |
| | | VQWKVDNALQSGNSQESVTEQD | GCTTTTCGGGGTCGGGATCCGGGACAGATT |
| | | SKDSTYSLSSTLTLSKADYEKHKV | TCACGCTCACAATCTCCTCGCTGGAACCCG |
| | | YACEVTHQGLSSPVTKSFNRGEC | AGGACTTCGCGGTCTACTATTGTCATCAGTC |
| | | (SEQ ID NO: 504) | ATCGAGGTTGCCTTTCACGTTTGGACCAGG |
| | | | GACCAAGGTGGACATTAAGCGTACGGTGGC |
| | | | TGCACCATCTGTCTTCATCTTCCCGCCATCT |
| | | | GATGAGCAGTTGAAATCTGGAACTGCCTCT |
| | | | GTTGTGTGCCTGCTGAATAACTTCTATCCCA |
| | | | GAGAGGCCAAAGTACAGTGGAAGGTGGAT |
| | | | AACGCCCTCCAATCGGGTAACTCCCAGGAG |
| | | | AGTGTCACAGAGCAGGACAGCAAGGACAG |
| | | | CACCTACAGCCTCAGCAGCACCCTGACGCT |
| | | | GAGCAAAGCAGACTACGAGAAACACAAAG |
| | | | TCTACGCCTGCGAAGTCACCCATCAGGGCC |
| | | | TGAGCTCGCCCGTCACAAAGAGCTTCAACA |
| | | | GGGGAGAGTGT (SEQ ID NO: 537) |
| iPS:421873 | LC-01 | EIVLTQSPATLSLSPGERATLSCRA | GAGATCGTACTTACTCAGTCACCCGCCACA |
| | | SQSVGRSLHWYQQKPGQAPRLLI | TTGTCCCTGAGCCCGGGTGAACGGGCGACC |
| | | KYASQSLSGIPARFSGSGSGTDFT | CTCAGCTGCCGAGCATCCCAGTCCGTCGGA |
| | | LTISSLEPEDFAVYYCHQSSRLPFT | CGATCATTGCACTGGTACCAACAAAAACCG |
| | | FGPGTKVDIKRTVAAPSVFIFPPSD | GGCCAGGCCCCCAGACTTCTGATCAAGTAT |
| | | EQLKSGTASVVCLLNNFYPREAK | GCGTCACAGAGCTTGTCGGGTATTCCCGCTC |
| | | VQWKVDNALQSGNSQESVTEQD | GCTTTTCGGGGTCGGGATCCGGGACAGATT |
| | | SKDSTYSLSSTLTLSKADYEKHKV | TCACGCTCACAATCTCCTCGCTGGAACCCG |
| | | YACEVTHQGLSSPVTKSFNRGEC | AGGACTTCGCGGTCTACTATTGTCATCAGTC |
| | | (SEQ ID NO: 504) | ATCGAGGTTGCCTTTCACGTTTGGACCAGG |
| | | | GACCAAGGTGGACATTAAGCGTACGGTGGC |
| | | | TGCACCATCTGTCTTCATCTTCCCGCCATCT |
| | | | GATGAGCAGTTGAAATCTGGAACTGCCTCT |
| | | | GTTGTGTGCCTGCTGAATAACTTCTATCCCA |
| | | | GAGAGGCCAAAGTACAGTGGAAGGTGGAT |
| | | | AACGCCCTCCAATCGGGTAACTCCCAGGAG |
| | | | AGTGTCACAGAGCAGGACAGCAAGGACAG |
| | | | CACCTACAGCCTCAGCAGCACCCTGACGCT |
| | | | GAGCAAAGCAGACTACGAGAAACACAAAG |
| | | | TCTACGCCTGCGAAGTCACCCATCAGGGCC |
| | | | TGAGCTCGCCCGTCACAAAGAGCTTCAACA |
| | | | GGGGAGAGTGT (SEQ ID NO: 537) |
| iPS:420889 | LC-03 | EIVLTQSPATLSLSPGERATLSCRA | GAGATCGTACTTACTCAGTCACCCGCCACA |
| | | SKSVGRSLHWYQQKPGQAPRLLI | TTGTCCCTGAGCCCGGGTGAACGGGCGACC |
| | | KYASQSLSGIPARFSGSGSGTDFT | CTCAGCTGCCGAGCATCCAAGTCCGTCGGA |
| | | LTISSLEPEDFAVYYCHQSSRLPFT | CGATCATTGCACTGGTACCAACAAAAACCG |
| | | FGPGTKVDIKRTVAAPSVFIFPPSD | GGCCAGGCCCCCAGACTTCTGATCAAGTAT |
| | | EQLKSGTASVVCLLNNFYPREAK | GCGTCACAGAGCTTGTCGGGTATTCCCGCTC |
| | | VQWKVDNALQSGNSQESVTEQD | GCTTTTCGGGGTCGGGATCCGGGACAGATT |
| | | SKDSTYSLSSTLTLSKADYEKHKV | TCACGCTCACAATCTCCTCGCTGGAACCCG |
| | | YACEVTHQGLSSPVTKSFNRGEC | AGGACTTCGCGGTCTACTATTGTCATCAGTC |
| | | (SEQ ID NO: 506) | ATCGAGGTTGCCTTTCACGTTTGGACCAGG |
| | | | GACCAAGGTGGACATTAAGCGTACGGTGGC |
| | | | TGCACCATCTGTCTTCATCTTCCCGCCATCT |
| | | | GATGAGCAGTTGAAATCTGGAACTGCCTCT |
| | | | GTTGTGTGCCTGCTGAATAACTTCTATCCCA |
| | | | GAGAGGCCAAAGTACAGTGGAAGGTGGAT |
| | | | AACGCCCTCCAATCGGGTAACTCCCAGGAG |
| | | | AGTGTCACAGAGCAGGACAGCAAGGACAG |
| | | | CACCTACAGCCTCAGCAGCACCCTGACGCT |
| | | | GAGCAAAGCAGACTACGAGAAACACAAAG |
| | | | TCTACGCCTGCGAAGTCACCCATCAGGGCC |
| | | | TGAGCTCGCCCGTCACAAAGAGCTTCAACA |
| | | | GGGGAGAGTGT (SEQ ID NO: 539) |
| iPS:421091 | LC-01 | EIVLTQSPATLSLSPGERATLSCRA | GAGATCGTACTTACTCAGTCACCCGCCACA |
| | | SQSVGRSLHWYQQKPGQAPRLLI | TTGTCCCTGAGCCCGGGTGAACGGGCGACC |
| | | KYASQSLSGIPARFSGSGSGTDFT | CTCAGCTGCCGAGCATCCCAGTCCGTCGGA |
| | | LTISSLEPEDFAVYYCHQSSRLPFT | CGATCATTGCACTGGTACCAACAAAAACCG |
| | | FGPGTKVDIKRTVAAPSVFIFPPSD | GGCCAGGCCCCCAGACTTCTGATCAAGTAT |
| | | EQLKSGTASVVCLLNNFYPREAK | GCGTCACAGAGCTTGTCGGGTATTCCCGCTC |
| | | VQWKVDNALQSGNSQESVTEQD | GCTTTTCGGGGTCGGGATCCGGGACAGATT |
| | | SKDSTYSLSSTLTLSKADYEKHKV | TCACGCTCACAATCTCCTCGCTGGAACCCG |
| | | YACEVTHQGLSSPVTKSFNRGEC | AGGACTTCGCGGTCTACTATTGTCATCAGTC |
| | | (SEQ ID NO: 504) | ATCGAGGTTGCCTTTCACGTTTGGACCAGG |
| | | | GACCAAGGTGGACATTAAGCGTACGGTGGC |
| | | | TGCACCATCTGTCTTCATCTTCCCGCCATCT |
| | | | GATGAGCAGTTGAAATCTGGAACTGCCTCT |
| | | | GTTGTGTGCCTGCTGAATAACTTCTATCCCA |
| | | | GAGAGGCCAAAGTACAGTGGAAGGTGGAT |
| | | | AACGCCCTCCAATCGGGTAACTCCCAGGAG |
| | | | AGTGTCACAGAGCAGGACAGCAAGGACAG |
| | | | CACCTACAGCCTCAGCAGCACCCTGACGCT |
| | | | GAGCAAAGCAGACTACGAGAAACACAAAG |
| | | | TCTACGCCTGCGAAGTCACCCATCAGGGCC |
| | | | TGAGCTCGCCCGTCACAAAGAGCTTCAACA |
| | | | GGGGAGAGTGT (SEQ ID NO: 537) |
| iPS:421051 | LC-03 | EIVLTQSPATLSLSPGERATLSCRA | GAGATCGTACTTACTCAGTCACCCGCCACA |
| | | SKSVGRSLHWYQQKPGQAPRLLI | TTGTCCCTGAGCCCGGGTGAACGGGCGACC |
| | | KYASQSLSGIPARFSGSGSGTDFT | CTCAGCTGCCGAGCATCCAAGTCCGTCGGA |
| | | LTISSLEPEDFAVYYCHQSSRLPFT | CGATCATTGCACTGGTACCAACAAAAACCG |
| | | FGPGTKVDIKRTVAAPSVFIFPPSD | GGCCAGGCCCCCAGACTTCTGATCAAGTAT |
| | | EQLKSGTASVVCLLNNFYPREAK | GCGTCACAGAGCTTGTCGGGTATTCCCGCTC |
| | | VQWKVDNALQSGNSQESVTEQD | GCTTTTCGGGGTCGGGATCCGGGACAGATT |
| | | SKDSTYSLSSTLTLSKADYEKHKV | TCACGCTCACAATCTCCTCGCTGGAACCCG |
| | | YACEVTHQGLSSPVTKSFNRGEC | AGGACTTCGCGGTCTACTATTGTCATCAGTC |
| | | (SEQ ID NO: 506) | ATCGAGGTTGCCTTTCACGTTTGGACCAGG |
| | | | GACCAAGGTGGACATTAAGCGTACGGTGGC |
| | | | TGCACCATCTGTCTTCATCTTCCCGCCATCT |
| | | | GATGAGCAGTTGAAATCTGGAACTGCCTCT |
| | | | GTTGTGTGCCTGCTGAATAACTTCTATCCCA |
| | | | GAGAGGCCAAAGTACAGTGGAAGGTGGAT |
| | | | AACGCCCTCCAATCGGGTAACTCCCAGGAG |
| | | | AGTGTCACAGAGCAGGACAGCAAGGACAG |
| | | | CACCTACAGCCTCAGCAGCACCCTGACGCT |
| | | | GAGCAAAGCAGACTACGAGAAACACAAAG |
| | | | TCTACGCCTGCGAAGTCACCCATCAGGGCC |
| | | | TGAGCTCGCCCGTCACAAAGAGCTTCAACA |
| | | | GGGGAGAGTGT (SEQ ID NO: 539) |
| iPS:480711 | LC-04 | EIVLTQSPATLSLSPGERATLSCRA | GAGATCGTACTTACTCAGTCACCCGCCACA |
| | | SKSVGWSLHWYQQKPGQAPRLLI | TTGTCCCTGAGCCCGGGTGAACGGGCGACC |
| | | KYASQSLSGIPARFSGSGSGTDFT | CTCAGCTGCCGAGCATCCAAATCCGTCGGG |
| | | LTISSLEPEDFAVYYCHQSSRLPFT | TGGAGCTTGCACTGGTACCAACAAAAACCG |
| | | FGPGTKVDIKRTVAAPSVFIFPPSD | GGCCAGGCCCCCAGACTTCTGATCAAGTAT |
| | | EQLKSGTASVVCLLNNFYPREAK | GCGTCACAGAGCTTGTCGGGTATTCCCGCTC |
| | | VQWKVDNALQSGNSQESVTEQD | GCTTTTCGGGGTCGGGATCCGGGACAGATT |
| | | SKDSTYSLSSTLTLSKADYEKHKV | TCACGCTCACAATCTCCTCGCTGGAACCCG |
| | | YACEVTHQGLSSPVTKSFNRGEC | AGGACTTCGCGGTCTACTATTGTCATCAGTC |
| | | (SEQ ID NO: 507) | ATCGAGGTTGCCTTTCACGTTTGGACCAGG |
| | | | GACCAAGGTGGACATTAAGCGTACGGTGGC |
| | | | TGCACCATCTGTCTTCATCTTCCCGCCATCT |
| | | | GATGAGCAGTTGAAATCTGGAACTGCCTCT |
| | | | GTTGTGTGCCTGCTGAATAACTTCTATCCCA |
| | | | GAGAGGCCAAAGTACAGTGGAAGGTGGAT |
| | | | AACGCCCTCCAATCGGGTAACTCCCAGGAG |
| | | | AGTGTCACAGAGCAGGACAGCAAGGACAG |
| | | | CACCTACAGCCTCAGCAGCACCCTGACGCT |
| | | | GAGCAAAGCAGACTACGAGAAACACAAAG |
| | | | TCTACGCCTGCGAAGTCACCCATCAGGGCC |
| | | | TGAGCTCGCCCGTCACAAAGAGCTTCAACA |
| | | | GGGGAGAGTGT (SEQ ID NO: 540) |
| iPS:480706 | LC-04 | EIVLTQSPATLSLSPGERATLSCRA | GAGATCGTACTTACTCAGTCACCCGCCACA |
| | | SKSVGWSLHWYQQKPGQAPRLLI | TTGTCCCTGAGCCCGGGTGAACGGGCGACC |
| | | KYASQSLSGIPARFSGSGSGTDFT | CTCAGCTGCCGAGCATCCAAATCCGTCGGG |
| | | LTISSLEPEDFAVYYCHQSSRLPFT | TGGAGCTTGCACTGGTACCAACAAAAACCG |
| | | FGPGTKVDIKRTVAAPSVFIFPPSD | GGCCAGGCCCCCAGACTTCTGATCAAGTAT |
| | | EQLKSGTASVVCLLNNFYPREAK | GCGTCACAGAGCTTGTCGGGTATTCCCGCTC |
| | | VQWKVDNALQSGNSQESVTEQD | GCTTTTCGGGGTCGGGATCCGGGACAGATT |
| | | SKDSTYSLSSTLTLSKADYEKHKV | TCACGCTCACAATCTCCTCGCTGGAACCCG |
| | | YACEVTHQGLSSPVTKSFNRGEC | AGGACTTCGCGGTCTACTATTGTCATCAGTC |
| | | (SEQ ID NO: 507) | ATCGAGGTTGCCTTTCACGTTTGGACCAGG |
| | | | GACCAAGGTGGACATTAAGCGTACGGTGGC |
| | | | TGCACCATCTGTCTTCATCTTCCCGCCATCT |
| | | | GATGAGCAGTTGAAATCTGGAACTGCCTCT |
| | | | GTTGTGTGCCTGCTGAATAACTTCTATCCCA |
| | | | GAGAGGCCAAAGTACAGTGGAAGGTGGAT |
| | | | AACGCCCTCCAATCGGGTAACTCCCAGGAG |
| | | | AGTGTCACAGAGCAGGACAGCAAGGACAG |
| | | | CACCTACAGCCTCAGCAGCACCCTGACGCT |
| | | | GAGCAAAGCAGACTACGAGAAACACAAAG |
| | | | TCTACGCCTGCGAAGTCACCCATCAGGGCC |
| | | | TGAGCTCGCCCGTCACAAAGAGCTTCAACA |
| | | | GGGGAGAGTGT (SEQ ID NO: 540) |
| iPS:480713 | LC-05 | EIVLTQSPATLSLSPGERATLSCRA | GAGATCGTACTTACTCAGTCACCCGCCACA |
| | | SKSVGYSLHWYQQKPGQAPRLLI | TTGTCCCTGAGCCCGGGTGAACGGGCGACC |
| | | KYASQSLSGIPARFSGSGSGTDFT | CTCAGCTGCCGAGCATCCAAATCCGTCGGG |
| | | LTISSLEPEDFAVYYCHQSSRLPFT | TACAGCTTGCACTGGTACCAACAAAAACCG |
| | | FGPGTKVDIKRTVAAPSVFIFPPSD | GGCCAGGCCCCCAGACTTCTGATCAAGTAT |
| | | EQLKSGTASVVCLLNNFYPREAK | GCGTCACAGAGCTTGTCGGGTATTCCCGCTC |
| | | VQWKVDNALQSGNSQESVTEQD | GCTTTTCGGGGTCGGGATCCGGGACAGATT |
| | | SKDSTYSLSSTLTLSKADYEKHKV | TCACGCTCACAATCTCCTCGCTGGAACCCG |
| | | YACEVTHQGLSSPVTKSFNRGEC | AGGACTTCGCGGTCTACTATTGTCATCAGTC |
| | | (SEQ ID NO: 508) | ATCGAGGTTGCCTTTCACGTTTGGACCAGG |
| | | | GACCAAGGTGGACATTAAGCGTACGGTGGC |
| | | | TGCACCATCTGTCTTCATCTTCCCGCCATCT |
| | | | GATGAGCAGTTGAAATCTGGAACTGCCTCT |
| | | | GTTGTGTGCCTGCTGAATAACTTCTATCCCA |
| | | | GAGAGGCCAAAGTACAGTGGAAGGTGGAT |
| | | | AACGCCCTCCAATCGGGTAACTCCCAGGAG |
| | | | AGTGTCACAGAGCAGGACAGCAAGGACAG |
| | | | CACCTACAGCCTCAGCAGCACCCTGACGCT |
| | | | GAGCAAAGCAGACTACGAGAAACACAAAG |
| | | | TCTACGCCTGCGAAGTCACCCATCAGGGCC |
| | | | TGAGCTCGCCCGTCACAAAGAGCTTCAACA |
| | | | GGGGAGAGTGT (SEQ ID NO: 541) |

19H8 variants

| | | | |
|---|---|---|---|
| 19H8 | LC-06 | DIQMTQSPSSLSASVGDRITITCRA | GACATCCAGATGACCCAGTCTCCATCCTCCC |
| | | SQSISRYLNWYQQKPGKAPKLLIY | TGTCTGCATCTGTAGGAGACAGAATCACCA |
| | | AASSLQSGIPSRFSGSGSGTDFTLT | TCACTTGCCGGGCAAGTCAGAGCATTAGCA |
| | | INSLQPEDFATYFCQQSYSPPFTFG | GGTATTTAAATTGGTATCAACAGAAACCAG |
| | | PGTKVDIKRTVAAPSVFIFPPSDEQ | GGAAAGCCCCTAAACTCCTGATCTATGCTG |
| | | LKSGTASVVCLLNNFYPREAKVQ | CATCCAGTTTGCAAAGTGGGATCCCATCAA |
| | | WKVDNALQSGNSQESVTEQDSK | GGTTCAGCGGCAGTGGATCTGGGACAGATT |
| | | DSTYSLSSTLTLSKADYEKHKVY | TCACTCTCACCATCAACAGTCTGCAACCTGA |
| | | ACEVTHQGLSSPVTKSFNRGEC | AGATTTTGCAACTTACTTCTGTCAACAGAGT |
| | | (SEQ ID NO: 509) | TACAGTCCCCCATTCACTTTCGGCCCTGGGA |
| | | | CCAAAGTGGATATCAAACGTACGGTGGCTG |
| | | | CACCATCTGTCTTCATCTTCCCGCCATCTGA |
| | | | TGAGCAGTTGAAATCTGGAACTGCCTCTGTT |
| | | | GTGTGCCTGCTGAATAACTTCTATCCCAGAG |
| | | | AGGCCAAAGTACAGTGGAAGGTGGATAAC |
| | | | GCCCTCCAATCGGGTAACTCCCAGGAGAGT |
| | | | GTCACAGAGCAGGACAGCAAGGACAGCAC |
| | | | CTACAGCCTCAGCAGCACCCTGACGCTGAG |
| | | | CAAAGCAGACTACGAGAAACACAAAGTCTA |
| | | | CGCCTGCGAAGTCACCCATCAGGGCCTGAG |
| | | | CTCGCCCGTCACAAAGAGCTTCAACAGGGG |
| | | | AGAGTGT (SEQ ID NO: 542) |
| iPS:448730 | LC-07 | DIQMTQSPSSLSASVGDRITITCRA | GACATCCAGATGACCCAGTCTCCATCCTCCC |
| | | SQMIARYLNWYQQKPGKAPKLLI | TGTCTGCATCTGTAGGAGACAGAATCACCA |
| | | YASYNLQSGIPSRFSGSGSGTDFT | TCACTTGCCGGGCAAGTCAGATGATTGCTC |
| | | LTINSLQPEDFATYFCQQAIINPYT | GTTACTTAAACTGGTATCAACAGAAACCAG |
| | | FGPGTKVDIKRTVAAPSVFIFPPSD | GGAAAGCCCCTAAACTCCTGATCTACGCTT |
| | | EQLKSGTASVVCLLNNFYPREAK | CTTACAACTTGCAAAGTGGGATCCCATCAA |
| | | VQWKVDNALQSGNSQESVTEQD | GGTTCAGCGGCAGTGGATCTGGGACAGATT |
| | | SKDSTYSLSSTLTLSKADYEKHKV | TCACTCTCACCATCAACAGTCTGCAACCTGA |
| | | YACEVTHQGLSSPVTKSFNRGEC | AGATTTTGCAACTTACTTCTGTCAACAGGCT |
| | | (SEQ ID NO: 510) | ATCATCAACCCATACACTTTCGGCCCTGGG |
| | | | ACCAAAGTGGATATCAAACGTACGGTGGCT |
| | | | GCACCATCTGTCTTCATCTTCCCGCCATCTG |
| | | | ATGAGCAGTTGAAATCTGGAACTGCCTCTG |
| | | | TTGTGTGCCTGCTGAATAACTTCTATCCCAG |
| | | | AGAGGCCAAAGTACAGTGGAAGGTGGATA |
| | | | ACGCCCTCCAATCGGGTAACTCCCAGGAGA |
| | | | GTGTCACAGAGCAGGACAGCAAGGACAGC |
| | | | ACCTACAGCCTCAGCAGCACCCTGACGCTG |
| | | | AGCAAAGCAGACTACGAGAAACACAAAGT |
| | | | CTACGCCTGCGAAGTCACCCATCAGGGCCT |
| | | | GAGCTCGCCCGTCACAAAGAGCTTCAACAG |
| | | | GGGAGAGTGT (SEQ ID NO: 543) |
| iPS:448195 | LC-08 | DIQMTQSPSSLSASVGDRITITCRA | GACATCCAGATGACCCAGTCTCCATCCTCCC |
| | | SQKIARYLVWYQQKPGKAPKLLI | TGTCTGCATCTGTAGGAGACAGAATCACCA |
| | | YAANMLQSGIPSRFSGSGSGTDFT | TCACTTGCCGGGCAAGTCAGAAAATTGCTC |
| | | LTINSLQPEDFATYFCQQSIQQPYT | GTTACTTAGTTTGGTATCAACAGAAACCAG |
| | | FGPGTKVDIKRTVAAPSVFIFPPSD | GGAAAGCCCCTAAACTCCTGATCTACGCTG |
| | | EQLKSGTASVVCLLNNFYPREAK | CTAACATGTTGCAAAGTGGGATCCCATCAA |
| | | VQWKVDNALQSGNSQESVTEQD | GGTTCAGCGGCAGTGGATCTGGGACAGATT |
| | | SKDSTYSLSSTLTLSKADYEKHKV | TCACTCTCACCATCAACAGTCTGCAACCTGA |
| | | YACEVTHQGLSSPVTKSFNRGEC | AGATTTTGCAACTTACTTCTGTCAACAGTCT |
| | | (SEQ ID NO: 511) | ATCCAGCAGCCATACACTTTCGGCCCTGGG |
| | | | ACCAAAGTGGATATCAAACGTACGGTGGCT |
| | | | GCACCATCTGTCTTCATCTTCCCGCCATCTG |
| | | | ATGAGCAGTTGAAATCTGGAACTGCCTCTG |
| | | | TTGTGTGCCTGCTGAATAACTTCTATCCCAG |
| | | | AGAGGCCAAAGTACAGTGGAAGGTGGATA |
| | | | ACGCCCTCCAATCGGGTAACTCCCAGGAGA |
| | | | GTGTCACAGAGCAGGACAGCAAGGACAGC |
| | | | ACCTACAGCCTCAGCAGCACCCTGACGCTG |
| | | | AGCAAAGCAGACTACGAGAAACACAAAGT |
| | | | CTACGCCTGCGAAGTCACCCATCAGGGCCT |
| | | | GAGCTCGCCCGTCACAAAGAGCTTCAACAG |
| | | | GGGAGAGTGT (SEQ ID NO: 544) |
| iPS:448788 | LC-09 | DIQMTQSPSSLSASVGDRITITCRA | GACATCCAGATGACCCAGTCTCCATCCTCCC |
| | | SQSISRYLNWYQQKPGKAPKLLIY | TGTCTGCATCTGTAGGAGACAGAATCACCA |
| | | AGRILQSGIPSRFSGSGSGTDFTLT | TCACTTGCCGGGCAAGTCAGAGCATTAGCA |
| | | INSLQPEDFATYFCQQAIINPYTFG | GGTATTTAAATTGGTATCAACAGAAACCAG |
| | | PGTKVDIKRTVAAPSVFIFPPSDEQ | GGAAAGCCCCTAAACTCCTGATCTACGCTG |
| | | LKSGTASVVCLLNNFYPREAKVQ | GTCGTATCTTGCAAAGTGGGATCCCATCAA |
| | | WKVDNALQSGNSQESVTEQDSK | GGTTCAGCGGCAGTGGATCTGGGACAGATT |
| | | DSTYSLSSTLTLSKADYEKHKVY | TCACTCTCACCATCAACAGTCTGCAACCTGA |
| | | ACEVTHQGLSSPVTKSFNRGEC | AGATTTTGCAACTTACTTCTGTCAACAGGCT |
| | | (SEQ ID NO: 512) | ATCATCAACCCATACACTTTCGGCCCTGGG |
| | | | ACCAAAGTGGATATCAAACGTACGGTGGCT |
| | | | GCACCATCTGTCTTCATCTTCCCGCCATCTG |
| | | | ATGAGCAGTTGAAATCTGGAACTGCCTCTG |
| | | | TTGTGTGCCTGCTGAATAACTTCTATCCCAG |
| | | | AGAGGCCAAAGTACAGTGGAAGGTGGATA |
| | | | ACGCCCTCCAATCGGGTAACTCCCAGGAGA |
| | | | GTGTCACAGAGCAGGACAGCAAGGACAGC |
| | | | ACCTACAGCCTCAGCAGCACCCTGACGCTG |
| | | | AGCAAAGCAGACTACGAGAAACACAAAGT |
| | | | CTACGCCTGCGAAGTCACCCATCAGGGCCT |
| | | | GAGCTCGCCCGTCACAAAGAGCTTCAACAG |
| | | | GGGAGAGTGT (SEQ ID NO: 545) |
| iPS:448901 | LC-10 | DIQMTQSPSSLSASVGDRITITCRA | GACATCCAGATGACCCAGTCTCCATCCTCCC |
| | | SQSISRYLNWYQQKPGKAPKLLIY | TGTCTGCATCTGTAGGAGACAGAATCACCA |
| | | ASYNLQSGIPSRFSGSGSGTDFTLT | TCACTTGCCGGGCAAGTCAGAGCATTAGCA |
| | | INSLQPEDFATYFCQQSIQQPYTF | GGTATTTAAATTGGTATCAACAGAAACCAG |
| | | GPGTKVDIKRTVAAPSVFIFPPSDE | GGAAAGCCCCTAAACTCCTGATCTACGCTT |
| | | QLKSGTASVVCLLNNFYPREAKV | CTTACAACTTGCAAAGTGGGATCCCATCAA |
| | | QWKVDNALQSGNSQESVTEQDS | GGTTCAGCGGCAGTGGATCTGGGACAGATT |
| | | KDSTYSLSSTLTLSKADYEKHKV | TCACTCTCACCATCAACAGTCTGCAACCTGA |
| | | YACEVTHQGLSSPVTKSFNRGEC | AGATTTTGCAACTTACTTCTGTCAACAGTCT |
| | | (SEQ ID NO: 513) | ATCCAGCAGCCATACACTTTCGGCCCTGGG |
| | | | ACCAAAGTGGATATCAAACGTACGGTGGCT |
| | | | GCACCATCTGTCTTCATCTTCCCGCCATCTG |
| | | | ATGAGCAGTTGAAATCTGGAACTGCCTCTG |
| | | | TTGTGTGCCTGCTGAATAACTTCTATCCCAG |
| | | | AGAGGCCAAAGTACAGTGGAAGGTGGATA |
| | | | ACGCCCTCCAATCGGGTAACTCCCAGGAGA |
| | | | GTGTCACAGAGCAGGACAGCAAGGACAGC |
| | | | ACCTACAGCCTCAGCAGCACCCTGACGCTG |
| | | | AGCAAAGCAGACTACGAGAAACACAAAGT |
| | | | CTACGCCTGCGAAGTCACCCATCAGGGCCT |
| | | | GAGCTCGCCCGTCACAAAGAGCTTCAACAG |
| | | | GGGAGAGTGT (SEQ ID NO: 546) |
| iPS:448689 | LC-11 | DIQMTQSPSSLSASVGDRITITCRA | GACATCCAGATGACCCAGTCTCCATCCTCCC |
| | | SQYIVRYLNWYQQKPGKAPKLLI | TGTCTGCATCTGTAGGAGACAGAATCACCA |
| | | YASYNLQSGIPSRFSGSGSGTDFT | TCACTTGCCGGGCAAGTCAGTACATTGTTCG |
| | | LTINSLQPEDFATYFCQQAIMAPY | TTACTTAAACTGGTATCAACAGAAACCAGG |
| | | TFGPGTKVDIKRTVAAPSVFIFPPS | GAAAGCCCCTAAACTCCTGATCTACGCTTCT |
| | | DEQLKSGTASVVCLLNNFYPREA | TACAACTTGCAAAGTGGGATCCCATCAAGG |
| | | KVQWKVDNALQSGNSQESVTEQ | TTCAGCGGCAGTGGATCTGGGACAGATTTC |
| | | DSKDSTYSLSSTLTLSKADYEKHK | ACTCTCACCATCAACAGTCTGCAACCTGAA |
| | | VYACEVTHQGLSSPVTKSFNRGE | GATTTTGCAACTTACTTCTGTCAACAGGCTA |
| | | C(SEQ ID NO: 514) | TCATGGCTCCATACACTTTCGGCCCTGGGAC |
| | | | CAAAGTGGATATCAAACGTACGGTGGCTG |
| | | | CACCATCTGTCTTCATCTTCCCGCCATCTGA |
| | | | TGAGCAGTTGAAATCTGGAACTGCCTCTGTT |
| | | | GTGTGCCTGCTGAATAACTTCTATCCCAGAG |
| | | | AGGCCAAAGTACAGTGGAAGGTGGATAAC |
| | | | GCCCTCCAATCGGGTAACTCCCAGGAGAGT |
| | | | GTCACAGAGCAGGACAGCAAGGACAGCAC |
| | | | CTACAGCCTCAGCAGCACCCTGACGCTGAG |
| | | | CAAAGCAGACTACGAGAAACACAAAGTCTA |
| | | | CGCCTGCGAAGTCACCCATCAGGGCCTGAG |
| | | | CTCGCCCGTCACAAAGAGCTTCAACAGGGG |
| | | | AGAGTGT (SEQ ID NO: 547) |
| iPS:448202 | LC-12 | DIQMTQSPSSLSASVGDRITITCRA | GACATCCAGATGACCCAGTCTCCATCCTCCC |
| | | SQSISRYLNWYQQKPGKAPKLLIF | TGTCTGCATCTGTAGGAGACAGAATCACCA |
| | | AGQRLQSGIPSRFSGSGSGTDFTL | TCACTTGCCGGGCAAGTCAGAGCATTAGCA |
| | | TINSLQPEDFATYFCQQAIGMPYT | GGTATTTAAATTGGTATCAACAGAAACCAG |
| | | FGPGTKVDIKRTVAAPSVFIFPPSD | GGAAAGCCCCTAAACTCCTGATCTTCGCTG |
| | | EQLKSGTASVVCLLNNFYPREAK | GTCAGCGTTTGCAAAGTGGGATCCCATCAA |
| | | VQWKVDNALQSGNSQESVTEQD | GGTTCAGCGGCAGTGGATCTGGGACAGATT |
| | | SKDSTYSLSSTLTLSKADYEKHKV | TCACTCTCACCATCAACAGTCTGCAACCTGA |
| | | YACEVTHQGLSSPVTKSFNRGEC | AGATTTTGCAACTTACTTCTGTCAACAGGCT |
| | | (SEQ ID NO: 515) | ATCGGTATGCCATACACTTTCGGCCCTGGG |
| | | | ACCAAAGTGGATATCAAACGTACGGTGGCT |
| | | | GCACCATCTGTCTTCATCTTCCCGCCATCTG |
| | | | ATGAGCAGTTGAAATCTGGAACTGCCTCTG |
| | | | TTGTGTGCCTGCTGAATAACTTCTATCCCAG |
| | | | AGAGGCCAAAGTACAGTGGAAGGTGGATA |
| | | | ACGCCCTCCAATCGGGTAACTCCCAGGAGA |
| | | | GTGTCACAGAGCAGGACAGCAAGGACAGC |
| | | | ACCTACAGCCTCAGCAGCACCCTGACGCTG |
| | | | AGCAAAGCAGACTACGAGAAACACAAAGT |
| | | | CTACGCCTGCGAAGTCACCCATCAGGGCCT |
| | | | GAGCTCGCCCGTCACAAAGAGCTTCAACAG |
| | | | GGGAGAGTGT (SEQ ID NO: 548) |
| iPS:452128 | LC-13 | DIQMTQSPSSLSASVGDRITITCRA | GACATCCAGATGACCCAGTCTCCATCCTCCC |
| | | SQYIVRYLNWYQQKPGKAPKLLI | TGTCTGCATCTGTAGGAGACAGAATCACCA |
| | | YAANMLQSGIPSRFSGSGSGTDFT | TCACTTGCCGGGCAAGTCAGTACATTGTTCG |
| | | LTINSLQPEDFATYFCQQAINQPY | TTACTTAAACTGGTATCAACAGAAACCAGG |
| | | TFGPGTKVDIKRTVAAPSVFIFPPS | GAAAGCCCCTAAACTCCTGATCTACGCTGC |
| | | DEQLKSGTASVVCLLNNFYPREA | TAACATGTTGCAAAGTGGGATCCCATCAAG |
| | | KVQWKVDNALQSGNSQESVIEQ | GTTCAGCGGCAGTGGATCTGGGACAGATTT |
| | | DSKDSTYSLSSTLTLSKADYEKHK | CACTCTCACCATCAACAGTCTGCAACCTGA |
| | | VYACEVTHQGLSSPVTKSFNRGE | AGATTTTGCAACTTACTTCTGTCAACAGGCT |
| | | C(SEQ ID NO: 516) | ATCAACCAGCCATACACTTTCGGCCCTGGG |
| | | | ACCAAAGTGGATATCAAACGTACGGTGGCT |
| | | | GCACCATCTGTCTTCATCTTCCCGCCATCTG |
| | | | ATGAGCAGTTGAAATCTGGAACTGCCTCTG |
| | | | TTGTGTGCCTGCTGAATAACTTCTATCCCAG |
| | | | AGAGGCCAAAGTACAGTGGAAGGTGGATA |
| | | | ACGCCCTCCAATCGGGTAACTCCCAGGAGA |
| | | | GTGTCACAGAGCAGGACAGCAAGGACAGC |
| | | | ACCTACAGCCTCAGCAGCACCCTGACGCTG |
| | | | AGCAAAGCAGACTACGAGAAACACAAAGT |
| | | | CTACGCCTGCGAAGTCACCCATCAGGGCCT |
| | | | GAGCTCGCCCGTCACAAAGAGCTTCAACAG |
| | | | GGGAGAGTGT (SEQ ID NO: 549) |
| iPS:448924 | LC-14 | DIQMTQSPSSLSASVGDRITITCRA | GACATCCAGATGACCCAGTCTCCATCCTCCC |
| | | SQPISRYLSWYQQKPGKAPKLLIF | TGTCTGCATCTGTAGGAGACAGAATCACCA |
| | | AGQRLQSGIPSRFSGSGSGTDFTL | TCACTTGCCGGGCAAGTCAGCCGATTTCTCG |
| | | TINSLQPEDFATYFCQQAISIPYTF | TTACTTATCTTGGTATCAACAGAAACCAGG |
| | | GPGTKVDIKRTVAAPSVFIFPPSDE | GAAAGCCCCTAAACTCCTGATCTTCGCTGGT |
| | | QLKSGTASVVCLLNNFYPREAKV | CAGCGTTTGCAAAGTGGGATCCCATCAAGG |
| | | QWKVDNALQSGNSQESVTEQDS | TTCAGCGGCAGTGGATCTGGGACAGATTTC |
| | | KDSTYSLSSTLTLSKADYEKHKV | ACTCTCACCATCAACAGTCTGCAACCTGAA |
| | | YACEVTHQGLSSPVTKSFNRGEC | GATTTTGCAACTTACTTCTGTCAACAGGCTA |
| | | (SEQ ID NO: 517) | TCTCTATCCCATACACTTTCGGCCCTGGGAC |
| | | | CAAAGTGGATATCAAACGTACGGTGGCTG |
| | | | CACCATCTGTCTTCATCTTCCCGCCATCTGA |
| | | | TGAGCAGTTGAAATCTGGAACTGCCTCTGTT |
| | | | GTGTGCCTGCTGAATAACTTCTATCCCAGAG |
| | | | AGGCCAAAGTACAGTGGAAGGTGGATAAC |
| | | | GCCCTCCAATCGGGTAACTCCCAGGAGAGT |
| | | | GTCACAGAGCAGGACAGCAAGGACAGCAC |
| | | | CTACAGCCTCAGCAGCACCCTGACGCTGAG |
| | | | CAAAGCAGACTACGAGAAACACAAAGTCTA |
| | | | CGCCTGCGAAGTCACCCATCAGGGCCTGAG |
| | | | CTCGCCCGTCACAAAGAGCTTCAACAGGGG |
| | | | AGAGTGT (SEQ ID NO: 550) |
| 3574 | LC-06 | DIQMTQSPSSLSASVGDRITITCRA | GACATCCAGATGACCCAGTCTCCATCCTCCC |
| | | SQSISRYLNWYQQKPGKAPKLLIY | TGTCTGCATCTGTAGGAGACAGAATCACCA |
| | | AASSLQSGIPSRFSGSGSGTDFTLT | TCACTTGCCGGGCAAGTCAGAGCATTAGCA |
| | | INSLQPEDFATYFCQQSYSPPFTFG | GGTATTTAAATTGGTATCAACAGAAACCAG |
| | | PGTKVDIKRTVAAPSVFIFPPSDEQ | GGAAAGCCCCTAAACTCCTGATCTATGCTG |
| | | LKSGTASVVCLLNNFYPREAKVQ | CATCCAGTTTGCAAAGTGGGATCCCATCAA |
| | | WKVDNALQSGNSQESVTEQDSK | GGTTCAGCGGCAGTGGATCTGGGACAGATT |
| | | DSTYSLSSTLTLSKADYEKHKVY | TCACTCTCACCATCAACAGTCTGCAACCTGA |
| | | ACEVTHQGLSSPVTKSFNRGEC | AGATTTTGCAACTTACTTCTGTCAACAGAGT |
| | | (SEQ ID NO: 509) | TACAGTCCCCCATTCACTTTCGGCCCTGGGA |
| | | | CCAAAGTGGATATCAAACGTACGGTGGCTG |
| | | | CACCATCTGTCTTCATCTTCCCGCCATCTGA |
| | | | TGAGCAGTTGAAATCTGGAACTGCCTCTGTT |
| | | | GTGTGCCTGCTGAATAACTTCTATCCCAGAG |
| | | | AGGCCAAAGTACAGTGGAAGGTGGATAAC |
| | | | GCCCTCCAATCGGGTAACTCCCAGGAGAGT |
| | | | GTCACAGAGCAGGACAGCAAGGACAGCAC |
| | | | CTACAGCCTCAGCAGCACCCTGACGCTGAG |
| | | | CAAAGCAGACTACGAGAAACACAAAGTCTA |
| | | | CGCCTGCGAAGTCACCCATCAGGGCCTGAG |
| | | | CTCGCCCGTCACAAAGAGCTTCAACAGGGG |
| | | | AGAGTGT (SEQ ID NO: 542) |
| 3575 | LC-15 | DIQMTQSPSSLSASVGDRITITCRA | GACATCCAGATGACCCAGTCTCCATCCTCCC |
| | | SQQIARYLNWYQQKPGKAPKLLI | TGTCTGCATCTGTAGGAGACAGAATCACCA |
| | | YASYNLQSGIPSRFSGSGSGTDFT | TCACTTGCCGGGCAAGTCAGCAGATTGCTC |
| | | LTINSLQPEDFATYFCQQAIIQPYT | GTTACTTAAACTGGTATCAACAGAAACCAG |
| | | FGPGTKVDIKRTVAAPSVFIFPPSD | GGAAAGCCCCTAAACTCCTGATCTACGCTT |
| | | EQLKSGTASVVCLLNNFYPREAK | CTTACAACTTGCAAAGTGGGATCCCATCAA |
| | | VQWKVDNALQSGNSQESVTEQD | GGTTCAGCGGCAGTGGATCTGGGACAGATT |
| | | SKDSTYSLSSTLTLSKADYEKHKV | TCACTCTCACCATCAACAGTCTGCAACCTGA |
| | | YACEVTHQGLSSPVTKSFNRGEC | AGATTTTGCAACTTACTTCTGTCAACAGGCT |
| | | (SEQ ID NO: 518) | ATCATCCAGCCATACACTTTCGGCCCTGGG |
| | | | ACCAAAGTGGATATCAAACGTACGGTGGCT |
| | | | GCACCATCTGTCTTCATCTTCCCGCCATCTG |
| | | | ATGAGCAGTTGAAATCTGGAACTGCCTCTG |
| | | | TTGTGTGCCTGCTGAATAACTTCTATCCCAG |
| | | | AGAGGCCAAAGTACAGTGGAAGGTGGATA |
| | | | ACGCCCTCCAATCGGGTAACTCCCAGGAGA |
| | | | GTGTCACAGAGCAGGACAGCAAGGACAGC |
| | | | ACCTACAGCCTCAGCAGCACCCTGACGCTG |
| | | | AGCAAAGCAGACTACGAGAAACACAAAGT |
| | | | CTACGCCTGCGAAGTCACCCATCAGGGCCT |
| | | | GAGCTCGCCCGTCACAAAGAGCTTCAACAG |
| | | | GGGAGAGTGT (SEQ ID NO: 551) |

TABLE 4B
Exemplary Anti-PAC1 Antibody Heavy Chain Sequences

| | | | |
|---|---|---|---|
| Antibody | HC | Heavy Chain Amino | |
| ID. | Group | Acid Sequence | Heavy Chain Nucleic Acid Sequence |

29G4 variants

| | | | |
|---|---|---|---|
| 29G4v10 | HC-01 | QVQLVESGGGVVQPGRS | CAGGTGCAGCTGGTGGAGTCTGGGGGAGGCGTGG |
| | | LRLSCAASGFTFSRFAMH | TCCAGCCTGGGAGGTCCCTGCGACTCTCCTGTGCAG |
| | | WVRQAPGKGLEWVAVIS | CCTCTGGATTCACCTTCAGTAGATTTGCCATGCACT |
| | | YDGGNKYYAESVKGRFT | GGGTCCGCCAGGCTCCAGGCAAGGGGCTGGAGTGG |
| | | ISRDNSKNTLYLQMNSLR | GTGGCAGTTATATCATATGATGGAGGAAATAAATA |
| | | AEDTALFYCARGYDVLT | CTATGCAGAGTCCGTGAAGGGCCGGTTCACCATCTC |
| | | GYPDYWGQGTLVTVSSA | CAGAGACAATTCCAAGAACACCCTGTATCTGCAAA |
| | | STKGPSVFPLAPSSKSTSG | TGAACAGCCTGAGAGCTGAGGACACGGCTCTGTTTT |
| | | GTAALGCLVKDYFPEPVT | ACTGTGCGAGAGGATACGATGTTTTGACTGGTTACC |
| | | VSWNSGALTSGVHTFPA | CCGACTACTGGGGCCAGGGAACCCTGGTCACCGTC |
| | | VLQSSGLYSLSSVVTVPSS | TCTAGTGCCTCCACCAAGGGCCCATCGGTCTTCCCC |
| | | SLGTQTYICNVNHKPSNT | CTGGCACCCTCCTCCAAGAGCACCTCTGGGGGCAC |
| | | KVDKKVEPKSCDKTHTC | AGCGGCCCTGGGCTGCCTGGTCAAGGACTACTTCCC |
| | | PPCPAPELLGGPSVFLFPP | CGAACCGGTGACGGTGTCGTGGAACTCAGGCGCCC |
| | | KPKDTLMISRTPEVTCVV | TGACCAGCGGCGTGCACACCTTCCCGGCTGTCCTAC |
| | | VDVSHEDPEVKFNWYVD | AGTCCTCAGGACTCTACTCCCTCAGCAGCGTGGTGA |
| | | GVEVHNAKTKPCEEQYG | CCGTGCCCTCCAGCAGCTTGGGCACCCAGACCTACA |
| | | STYRCVSVLTVLHQDWL | TCTGCAACGTGAATCACAAGCCCAGCAACACCAAG |
| | | NGKEYKCKVSNKALPAPI | GTGGACAAGAAAGTTGAGCCCAAATCTTGTGACAA |
| | | EKTISKAKGQPREPQVYT | AACTCACACATGCCCACCGTGCCCAGCACCTGAACT |
| | | LPPSREEMTKNQVSLTCL | CCTGGGGGGACCGTCAGTCTTCCTCTTCCCCCCAAA |
| | | VKGFYPSDIAVEWESNGQ | ACCCAAGGACACCCTCATGATCTCCCGGACCCCTGA |
| | | PENNYKTTPPVLDSDGSF | GGTCACATGCGTGGTGGTGGACGTGAGCCACGAAG |
| | | FLYSKLTVDKSRWQQGN | ACCCTGAGGTCAAGTTCAACTGGTACGTGGACGGC |
| | | VFSCSVMHEALHNHYTQ | GTGGAGGTGCATAATGCCAAGACAAAGCCGTGCGA |
| | | KSLSLSPGK (SEQ ID | GGAGCAGTACGGCAGCACGTACCGTTGCGTCAGCG |
| | | NO: 519) | TCCTCACCGTCCTGCACCAGGACTGGCTGAATGGCA |
| | | | AGGAGTACAAGTGCAAGGTGTCCAACAAAGCCCTC |
| | | | CCAGCCCCCATCGAGAAAACCATCTCCAAAGCCAA |
| | | | AGGGCAGCCCCGAGAACCACAGGTGTACACCCTGC |
| | | | CCCCATCCCGGGAGGAGATGACCAAGAACCAGGTC |
| | | | AGCCTGACCTGCCTGGTCAAAGGCTTCTATCCCAGC |
| | | | GACATCGCCGTGGAGTGGGAGAGCAATGGGCAGCC |
| | | | GGAGAACAACTACAAGACCACGCCTCCCGTGCTGG |
| | | | ACTCCGACGGCTCCTTCTTCCTCTATAGCAAGCTCA |
| | | | CCGTGGACAAGAGCAGGTGGCAGCAGGGGAACGTC |
| | | | TTCTCATGCTCCGTGATGCATGAGGCTCTGCACAAC |
| | | | CACTACACGCAGAAGAGCCTCTCCCTGTCTCCGGGT |
| | | | AAA (SEQ ID NO: 552) |
| 29G4v22 | HC-02 | QVQLVESGAEVVKPGAS | CAAGTTCAGTTGGTGGAGTCTGGAGCCGAAGTAGT |
| | | VKVSCKASGFTFSRFAMH | AAAGCCAGGAGCTTCAGTGAAAGTCTCTTGTAAAG |
| | | WVRQAPGQGLEWMGVIS | CAAGTGGATTCACGTTTAGCCGCTTTGCCATGCATT |
| | | YDGGNKYYAESVKGRVT | GGGTGCGGCAAGCTCCCGGTCAGGGGTTGGAGTGG |
| | | MTRDTSTSTLYMELSSLR | ATGGGAGTTATTAGCTATGACGGGGGCAATAAGTA |
| | | SEDTAVYYCARGYDVLT | CTACGCCGAGTCTGTTAAGGGTCGGGTCACAATGA |
| | | GYPDYWGQGTLVTVSSA | CACGGGACACCTCAACCAGTACACTCTATATGGAA |
| | | STKGPSVFPLAPSSKSTSG | CTGTCTAGCCTGAGATCCGAGGACACCGCTGTGTAT |
| | | GTAALGCLVKDYFPEPVT | TATTGCGCTAGGGGGTACGATGTATTGACGGGTTAT |
| | | VSWNSGALTSGVHTFPA | CCTGATTACTGGGGGCAGGGGACACTCGTAACCGT |
| | | VLQSSGLYSLSSVVTVPSS | CTCTAGTGCCTCCACCAAGGGCCCATCGGTCTTCCC |
| | | SLGTQTYICNVNHKPSNT | CCTGGCACCCTCCTCCAAGAGCACCTCTGGGGGCAC |
| | | KVDKKVEPKSCDKTHTC | AGCGGCCCTGGGCTGCCTGGTCAAGGACTACTTCCC |
| | | PPCPAPELLGGPSVFLFPP | CGAACCGGTGACGGTGTCGTGGAACTCAGGCGCCC |
| | | KPKDTLMISRTPEVTCVV | TGACCAGCGGCGTGCACACCTTCCCGGCTGTCCTAC |
| | | VDVSHEDPEVKFNWYVD | AGTCCTCAGGACTCTACTCCCTCAGCAGCGTGGTGA |
| | | GVEVHNAKTKPREEQYG | CCGTGCCCTCCAGCAGCTTGGGCACCCAGACCTACA |
| | | STYRVVSVLTVLHQDWL | TCTGCAACGTGAATCACAAGCCCAGCAACACCAAG |
| | | NGKEYKCKVSNKALPAPI | GTGGACAAGAAAGTTGAGCCCAAATCTTGTGACAA |
| | | EKTISKAKGQPREPQVYT | AACTCACACATGCCCACCGTGCCCAGCACCTGAACT |
| | | LPPSREEMTKNQVSLTCL | CCTGGGGGGACCGTCAGTCTTCCTCTTCCCCCCAAA |
| | | VKGFYPSDIAVEWESNGQ | ACCCAAGGACACCCTCATGATCTCCCGGACCCCTGA |
| | | PENNYKTTPPVLDSDGSF | GGTCACATGCGTGGTGGTGGACGTGAGCCACGAAG |
| | | FLYSKLTVDKSRWQQGN | ACCCTGAGGTCAAGTTCAACTGGTACGTGGACGGC |
| | | VFSCSVMHEALHNHYTQ | GTGGAGGTGCATAATGCCAAGACAAAGCCGCGGGA |
| | | KSLSLSPGK (SEQ ID | GGAGCAGTACGGCAGCACGTACCGTGTGGTCAGCG |
| | | NO: 520) | TCCTCACCGTCCTGCACCAGGACTGGCTGAATGGCA |
| | | | AGGAGTACAAGTGCAAGGTCTCCAACAAAGCCCTC |
| | | | CCAGCCCCCATCGAGAAAACCATCTCCAAAGCCAA |
| | | | AGGGCAGCCCCGAGAACCACAGGTGTACACCCTGC |
| | | | CCCCATCCCGGGAGGAGATGACCAAGAACCAGGTC |
| | | | AGCCTGACCTGCCTGGTCAAAGGCTTCTATCCCAGC |
| | | | GACATCGCCGTGGAGTGGGAGAGCAATGGGCAGCC |
| | | | GGAGAACAACTACAAGACCACGCCTCCCGTGCTGG |
| | | | ACTCCGACGGCTCCTTCTTCCTCTATAGCAAGCTCA |
| | | | CCGTGGACAAGAGCAGGTGGCAGCAGGGGAACGTC |
| | | | TTCTCATGCTCCGTGATGCATGAGGCTCTGCACAAC |
| | | | CACTACACGCAGAAGAGCCTCTCCCTGTCTCCGGGT |
| | | | AAA (SEQ ID NO: 553) |
| iPS:420653 | HC-03 | QVQLVESGGGVVQPGRS | CAGGTGCAGCTGGTGGAGTCTGGGGGAGGCGTGGT |
| | | LRLSCAASGFTFSRFAMH | CCAGCCTGGGAGGTCCCTGCGACTCTCCTGTGCAGC |
| | | WVRQAPGKGLEWVAVIS | CTCTGGATTCACCTTCAGTAGATTTGCCATGCACTG |
| | | YIGGNKYYAESVKGRFTI | GGTCCGCCAGGCTCCAGGCAAGGGGCTGGAGTGGG |
| | | SRDNSKNTLYLQMNSLR | TGGCAGTTATATCATATATCGGAGGAAATAAATACT |
| | | AEDTALFYCARGYDVLT | ATGCAGAGTCCGTGAAGGGCCGGTTCACCATCTCC |
| | | GYPDYWGQGTLVTVSSA | AGAGACAATTCCAAGAACACCCTGTATCTGCAAAT |
| | | STKGPSVFPLAPSSKSTSG | GAACAGCCTGAGAGCTGAGGACACGGCTCTGTTTT |
| | | GTAALGCLVKDYFPEPVT | ACTGTGCGAGAGGATACGATGTTTTGACTGGTTACC |
| | | VSWNSGALTSGVHTFPA | CCGACTACTGGGGCCAGGGAACCCTGGTCACCGTC |
| | | VLQSSGLYSLSSVVTVPSS | TCTAGTGCCTCCACCAAGGGCCCATCGGTCTTCCCC |
| | | SLGTQTYICNVNHKPSNT | CTGGCACCCTCCTCCAAGAGCACCTCTGGGGGCAC |
| | | KVDKKVEPKSCDKTHTC | AGCGGCCCTGGGCTGCCTGGTCAAGGACTACTTCCC |
| | | PPCPAPELLGGPSVFLFPP | CGAACCGGTGACGGTGTCGTGGAACTCAGGCGCCC |
| | | KPKDTLMISRTPEVTCVV | TGACCAGCGGCGTGCACACCTTCCCGGCTGTCCTAC |
| | | VDVSHEDPEVKFNWYVD | AGTCCTCAGGACTCTACTCCCTCAGCAGCGTGGTGA |
| | | GVEVHNAKTKPCEEQYG | CCGTGCCCTCCAGCAGCTTGGGCACCCAGACCTACA |
| | | STYRCVSVLTVLHQDWL | TCTGCAACGTGAATCACAAGCCCAGCAACACCAAG |
| | | NGKEYKCKVSNKALPAPI | GTGGACAAGAAAGTTGAGCCCAAATCTTGTGACAA |
| | | EKTISKAKGQPREPQVYT | AACTCACACATGCCCACCGTGCCCAGCACCTGAACT |
| | | LPPSREEMTKNQVSLTCL | CCTGGGGGGACCGTCAGTCTTCCTCTTCCCCCCAAA |
| | | VKGFYPSDIAVEWESNGQ | ACCCAAGGACACCCTCATGATCTCCCGGACCCCTGA |
| | | PENNYKTTPPVLDSDGSF | GGTCACATGCGTGGTGGTGGACGTGAGCCACGAAG |
| | | FLYSKLTVDKSRWQQGN | ACCCTGAGGTCAAGTTCAACTGGTACGTGGACGGC |
| | | VFSCSVMHEALHNHYTQ | GTGGAGGTGCATAATGCCAAGACAAAGCCGTGCGA |
| | | KSLSLSPGK (SEQ ID | GGAGCAGTACGGCAGCACGTACCGTTGCGTCAGCG |
| | | NO: 521) | TCCTCACCGTCCTGCACCAGGACTGGCTGAATGGCA |
| | | | AGGAGTACAAGTGCAAGGTGTCCAACAAAGCCCTC |
| | | | CCAGCCCCCATCGAGAAAACCATCTCCAAAGCCAA |
| | | | AGGGCAGCCCCGAGAACCACAGGTGTACACCCTGC |
| | | | CCCCATCCCGGGAGGAGATGACCAAGAACCAGGTC |
| | | | AGCCTGACCTGCCTGGTCAAAGGCTTCTATCCCAGC |
| | | | GACATCGCCGTGGAGTGGGAGAGCAATGGGCAGCC |
| | | | GGAGAACAACTACAAGACCACGCCTCCCGTGCTGG |
| | | | ACTCCGACGGCTCCTTCTTCCTCTATAGCAAGCTCA |
| | | | CCGTGGACAAGAGCAGGTGGCAGCAGGGGAACGTC |
| | | | TTCTCATGCTCCGTGATGCATGAGGCTCTGCACAAC |
| | | | CACTACACGCAGAAGAGCCTCTCCCTGTCTCCGGGT |
| | | | AAA (SEQ ID NO: 554) |
| iPS:420845 | HC-04 | QVQLVESGGGVVQPGRS | CAGGTGCAGCTGGTGGAGTCTGGGGGAGGCGTGGT |
| | | LRLSCAASGFTFSRFAMH | CCAGCCTGGGAGGTCCCTGCGACTCTCCTGTGCAGC |
| | | WVRQAPGKGLEWVAVIS | CTCTGGATTCACCTTCAGTAGATTTGCCATGCACTG |
| | | YQGRNKYYAESVKGRFTI | GGTCCGCCAGGCTCCAGGCAAGGGGCTGGAGTGGG |
| | | SRDNSKNTLYLQMNSLR | TGGCAGTTATATCATATCAGGGACGCAATAAATACT |
| | | AEDTALFYCARGYDVLT | ATGCAGAGTCCGTGAAGGGCCGGTTCACCATCTCC |
| | | GYPDYWGQGTLVTVSSA | AGAGACAATTCCAAGAACACCCTGTATCTGCAAAT |
| | | STKGPSVFPLAPSSKSTSG | GAACAGCCTGAGAGCTGAGGACACGGCTCTGTTTT |
| | | GTAALGCLVKDYFPEPVT | ACTGTGCGAGAGGATACGATGTTTTGACTGGTTACC |
| | | VSWNSGALTSGVHTFPA | CCGACTACTGGGGCCAGGGAACCCTGGTCACCGTC |
| | | VLQSSGLYSLSSVVTVPSS | TCTAGTGCCTCCACCAAGGGCCCATCGGTCTTCCCC |
| | | SLGTQTYICNVNHKPSNT | CTGGCACCCTCCTCCAAGAGCACCTCTGGGGGCAC |
| | | KVDKKVEPKSCDKTHTC | AGCGGCCCTGGGCTGCCTGGTCAAGGACTACTTCCC |
| | | PPCPAPELLGGPSVFLFPP | CGAACCGGTGACGGTGTCGTGGAACTCAGGCGCCC |
| | | KPKDTLMISRTPEVTCVV | TGACCAGCGGCGTGCACACCTTCCCGGCTGTCCTAC |
| | | VDVSHEDPEVKFNWYVD | AGTCCTCAGGACTCTACTCCCTCAGCAGCGTGGTGA |
| | | GVEVHNAKTKPCEEQYG | CCGTGCCCTCCAGCAGCTTGGGCACCCAGACCTACA |
| | | STYRCVSVLTVLHQDWL | TCTGCAACGTGAATCACAAGCCCAGCAACACCAAG |
| | | NGKEYKCKVSNKALPAPI | GTGGACAAGAAAGTTGAGCCCAAATCTTGTGACAA |
| | | EKTISKAKGQPREPQVYT | AACTCACACATGCCCACCGTGCCCAGCACCTGAACT |
| | | LPPSREEMTKNQVSLTCL | CCTGGGGGGACCGTCAGTCTTCCTCTTCCCCCCAAA |
| | | VKGFYPSDIAVEWESNGQ | ACCCAAGGACACCCTCATGATCTCCCGGACCCCTGA |
| | | PENNYKTTPPVLDSDGSF | GGTCACATGCGTGGTGGTGGACGTGAGCCACGAAG |
| | | FLYSKLTVDKSRWQQGN | ACCCTGAGGTCAAGTTCAACTGGTACGTGGACGGC |
| | | VFSCSVMHEALHNHYTQ | GTGGAGGTGCATAATGCCAAGACAAAGCCGTGCGA |
| | | KSLSLSPGK (SEQ ID | GGAGCAGTACGGCAGCACGTACCGTTGCGTCAGCG |
| | | NO: 522) | TCCTCACCGTCCTGCACCAGGACTGGCTGAATGGCA |
| | | | AGGAGTACAAGTGCAAGGTGTCCAACAAAGCCCTC |
| | | | CCAGCCCCCATCGAGAAAACCATCTCCAAAGCCAA |
| | | | AGGGCAGCCCCGAGAACCACAGGTGTACACCCTGC |
| | | | CCCCATCCCGGGAGGAGATGACCAAGAACCAGGTC |
| | | | AGCCTGACCTGCCTGGTCAAAGGCTTCTATCCCAGC |
| | | | GACATCGCCGTGGAGTGGGAGAGCAATGGGCAGCC |
| | | | GGAGAACAACTACAAGACCACGCCTCCCGTGCTGG |
| | | | ACTCCGACGGCTCCTTCTTCCTCTATAGCAAGCTCA |
| | | | CCGTGGACAAGAGCAGGTGGCAGCAGGGGAACGTC |
| | | | TTCTCATGCTCCGTGATGCATGAGGCTCTGCACAAC |
| | | | CACTACACGCAGAAGAGCCTCTCCCTGTCTCCGGGT |
| | | | AAA (SEQ ID NO: 555) |
| iPS:420943 | HC-05 | QVQLVESGGGVVQPGRS | CAGGTGCAGCTGGTGGAGTCTGGGGGAGGCGTGGT |
| | | LRLSCAASGFTFSRFAMH | CCAGCCTGGGAGGTCCCTGCGACTCTCCTGTGCAGC |
| | | WVRQAPGKGLEWVAVIS | CTCTGGATTCACCTTCAGTAGATTTGCCATGCACTG |
| | | YQGNNKYYARSVKGRFT | GGTCCGCCAGGCTCCAGGCAAGGGGCTGGAGTGGG |
| | | ISRDNSKNTLYLQMNSLR | TGGCAGTTATATCATATCAGGGAAACAATAAATAC |
| | | AEDTALFYCARGYDVLT | TATGCACGCTCCGTGAAGGGCCGGTTCACCATCTCC |
| | | GYPDYWGQGTLVTVSSA | AGAGACAATTCCAAGAACACCCTGTATCTGCAAAT |
| | | STKGPSVFPLAPSSKSTSG | GAACAGCCTGAGAGCTGAGGACACGGCTCTGTTTT |
| | | GTAALGCLVKDYFPEPVT | ACTGTGCGAGAGGATACGATGTTTTGACTGGTTACC |
| | | VSWNSGALTSGVHTFPA | CCGACTACTGGGGCCAGGGAACCCTGGTCACCGTC |
| | | VLQSSGLYSLSSVVTVPSS | TCTAGTGCCTCCACCAAGGGCCCATCGGTCTTCCCC |
| | | SLGTQTYICNVNHKPSNT | CTGGCACCCTCCTCCAAGAGCACCTCTGGGGGCAC |
| | | KVDKKVEPKSCDKTHTC | AGCGGCCCTGGGCTGCCTGGTCAAGGACTACTTCCC |
| | | PPCPAPELLGGPSVFLFPP | CGAACCGGTGACGGTGTCGTGGAACTCAGGCGCCC |
| | | KPKDTLMISRTPEVTCVV | TGACCAGCGGCGTGCACACCTTCCCGGCTGTCCTAC |
| | | VDVSHEDPEVKFNWYVD | AGTCCTCAGGACTCTACTCCCTCAGCAGCGTGGTGA |
| | | GVEVHNAKTKPCEEQYG | CCGTGCCCTCCAGCAGCTTGGGCACCCAGACCTACA |
| | | STYRCVSVLTVLHQDWL | TCTGCAACGTGAATCACAAGCCCAGCAACACCAAG |
| | | NGKEYKCKVSNKALPAPI | GTGGACAAGAAAGTTGAGCCCAAATCTTGTGACAA |
| | | EKTISKAKGQPREPQVYT | AACTCACACATGCCCACCGTGCCCAGCACCTGAACT |
| | | LPPSREEMTKNQVSLTCL | CCTGGGGGGACCGTCAGTCTTCCTCTTCCCCCCAAA |
| | | VKGFYPSDIAVEWESNGQ | ACCCAAGGACACCCTCATGATCTCCCGGACCCCTGA |
| | | PENNYKTTPPVLDSDGSF | GGTCACATGCGTGGTGGTGGACGTGAGCCACGAAG |
| | | FLYSKLTVDKSRWQQGN | ACCCTGAGGTCAAGTTCAACTGGTACGTGGACGGC |
| | | VFSCSVMHEALHNHYTQ | GTGGAGGTGCATAATGCCAAGACAAAGCCGTGCGA |
| | | KSLSLSPGK (SEQ ID | GGAGCAGTACGGCAGCACGTACCGTTGCGTCAGCG |
| | | NO: 523) | TCCTCACCGTCCTGCACCAGGACTGGCTGAATGGCA |
| | | | AGGAGTACAAGTGCAAGGTGTCCAACAAAGCCCTC |
| | | | CCAGCCCCCATCGAGAAAACCATCTCCAAAGCCAA |
| | | | AGGGCAGCCCCGAGAACCACAGGTGTACACCCTGC |
| | | | CCCCATCCCGGGAGGAGATGACCAAGAACCAGGTC |
| | | | AGCCTGACCTGCCTGGTCAAAGGCTTCTATCCCAGC |
| | | | GACATCGCCGTGGAGTGGGAGAGCAATGGGCAGCC |
| | | | GGAGAACAACTACAAGACCACGCCTCCCGTGCTGG |
| | | | ACTCCGACGGCTCCTTCTTCCTCTATAGCAAGCTCA |
| | | | CCGTGGACAAGAGCAGGTGGCAGCAGGGGAACGTC |
| | | | TTCTCATGCTCCGTGATGCATGAGGCTCTGCACAAC |
| | | | CACTACACGCAGAAGAGCCTCTCCCTGTCTCCGGGT |
| | | | AAA (SEQ ID NO: 556) |
| iPS:421873 | HC-06 | QVQLVESGGGVVQPGRS | CAGGTGCAGCTGGTGGAGTCTGGGGGAGGCGTGGT |
| | | LRLSCAASGFTFSKFAMH | CCAGCCTGGGAGGTCCCTGCGACTCTCCTGTGCAGC |
| | | WVRQAPGKGLEWVAVIS | CTCTGGATTCACCTTCAGTAAGTTTGCCATGCACTG |
| | | YRGGNKYYAESVKGRFTI | GGTCCGCCAGGCTCCAGGCAAGGGGCTGGAGTGGG |
| | | SRDNSKNTLYLQMNSLR | TGGCAGTTATATCATATCGCGGAGGAAATAAATAC |
| | | AEDTALFYCARGYDLLT | TATGCAGAGTCCGTGAAGGGCCGGTTCACCATCTCC |
| | | GYPDYWGQGTLVTVSSA | AGAGACAATTCCAAGAACACCCTGTATCTGCAAAT |
| | | STKGPSVFPLAPSSKSTSG | GAACAGCCTGAGAGCTGAGGACACGGCTCTGTTTT |
| | | GTAALGCLVKDYFPEPVT | ACTGTGCGAGAGGATACGATCTGTTGACTGGTTACC |
| | | VSWNSGALTSGVHTFPA | CCGACTACTGGGGCCAGGGAACCCTGGTCACCGTC |
| | | VLQSSGLYSLSSVVTVPSS | TCTAGTGCCTCCACCAAGGGCCCATCGGTCTTCCCC |
| | | SLGTQTYICNVNHKPSNT | CTGGCACCCTCCTCCAAGAGCACCTCTGGGGGCAC |
| | | KVDKKVEPKSCDKTHTC | AGCGGCCCTGGGCTGCCTGGTCAAGGACTACTTCCC |
| | | PPCPAPELLGGPSVFLFPP | CGAACCGGTGACGGTGTCGTGGAACTCAGGCGCCC |
| | | KPKDTLMISRTPEVTCVV | TGACCAGCGGCGTGCACACCTTCCCGGCTGTCCTAC |
| | | VDVSHEDPEVKFNWYVD | AGTCCTCAGGACTCTACTCCCTCAGCAGCGTGGTGA |
| | | GVEVHNAKTKPCEEQYG | CCGTGCCCTCCAGCAGCTTGGGCACCCAGACCTACA |
| | | STYRCVSVLTVLHQDWL | TCTGCAACGTGAATCACAAGCCCAGCAACACCAAG |
| | | NGKEYKCKVSNKALPAPI | GTGGACAAGAAAGTTGAGCCCAAATCTTGTGACAA |
| | | EKTISKAKGQPREPQVYT | AACTCACACATGCCCACCGTGCCCAGCACCTGAACT |
| | | LPPSREEMTKNQVSLTCL | CCTGGGGGGACCGTCAGTCTTCCTCTTCCCCCCAAA |
| | | VKGFYPSDIAVEWESNGQ | ACCCAAGGACACCCTCATGATCTCCCGGACCCCTGA |
| | | PENNYKTTPPVLDSDGSF | GGTCACATGCGTGGTGGTGGACGTGAGCCACGAAG |
| | | FLYSKLTVDKSRWQQGN | ACCCTGAGGTCAAGTTCAACTGGTACGTGGACGGC |
| | | VFSCSVMHEALHNHYTQ | GTGGAGGTGCATAATGCCAAGACAAAGCCGTGCGA |
| | | KSLSLSPGK (SEQ ID | GGAGCAGTACGGCAGCACGTACCGTTGCGTCAGCG |
| | | NO: 524) | TCCTCACCGTCCTGCACCAGGACTGGCTGAATGGCA |
| | | | AGGAGTACAAGTGCAAGGTGTCCAACAAAGCCCTC |
| | | | CCAGCCCCCATCGAGAAAACCATCTCCAAAGCCAA |
| | | | AGGGCAGCCCCGAGAACCACAGGTGTACACCCTGC |
| | | | CCCCATCCCGGGAGGAGATGACCAAGAACCAGGTC |
| | | | AGCCTGACCTGCCTGGTCAAAGGCTTCTATCCCAGC |
| | | | GACATCGCCGTGGAGTGGGAGAGCAATGGGCAGCC |
| | | | GGAGAACAACTACAAGACCACGCCTCCCGTGCTGG |
| | | | ACTCCGACGGCTCCTTCTTCCTCTATAGCAAGCTCA |
| | | | CCGTGGACAAGAGCAGGTGGCAGCAGGGGAACGTC |
| | | | TTCTCATGCTCCGTGATGCATGAGGCTCTGCACAAC |
| | | | CACTACACGCAGAAGAGCCTCTCCCTGTCTCCGGGT |
| | | | AAA (SEQ ID NO: 557) |
| iPS:420889 | HC-07 | QVQLVESGGGVVQPGRS | CAGGTGCAGCTGGTGGAGTCTGGGGGAGGCGTGGT |
| | | LRLSCAASGFTFSRFAMH | CCAGCCTGGGAGGTCCCTGCGACTCTCCTGTGCAGC |
| | | WVRQAPGKGLEWVAVIS | CTCTGGATTCACCTTCAGTAGATTTGCCATGCACTG |
| | | YIGGNKYYAESVKGRFTI | GGTCCGCCAGGCTCCAGGCAAGGGGCTGGAGTGGG |
| | | SRDNSKNTLYLQMNSLR | TGGCAGTTATATCATATATCGGAGGAAATAAATACT |
| | | AEDTALFYCARGYDILTG | ATGCAGAGTCCGTGAAGGGCCGGTTCACCATCTCC |
| | | YPDYWGQGTLVTVSSAS | AGAGACAATTCCAAGAACACCCTGTATCTGCAAAT |
| | | TKGPSVFPLAPSSKSTSGG | GAACAGCCTGAGAGCTGAGGACACGGCTCTGTTTT |
| | | TAALGCLVKDYFPEPVTV | ACTGTGCGAGAGGATACGATATCTTGACTGGTTACC |
| | | SWNSGALTSGVHTFPAVL | CCGACTACTGGGGCCAGGGAACCCTGGTCACCGTC |
| | | QSSGLYSLSSVVTVPSSSL | TCTAGTGCCTCCACCAAGGGCCCATCGGTCTTCCCC |
| | | GTQTYICNVNHKPSNTKV | CTGGCACCCTCCTCCAAGAGCACCTCTGGGGGCAC |
| | | DKKVEPKSCDKTHTCPPC | AGCGGCCCTGGGCTGCCTGGTCAAGGACTACTTCCC |
| | | PAPELLGGPSVFLFPPKPK | CGAACCGGTGACGGTGTCGTGGAACTCAGGCGCCC |
| | | DTLMISRTPEVTCVVVDV | TGACCAGCGGCGTGCACACCTTCCCGGCTGTCCTAC |
| | | SHEDPEVKFNWYVDGVE | AGTCCTCAGGACTCTACTCCCTCAGCAGCGTGGTGA |
| | | VHNAKTKPCEEQYGSTY | CCGTGCCCTCCAGCAGCTTGGGCACCCAGACCTACA |
| | | RCVSVLTVLHQDWLNGK | TCTGCAACGTGAATCACAAGCCCAGCAACACCAAG |
| | | EYKCKVSNKALPAPIEKTI | GTGGACAAGAAAGTTGAGCCCAAATCTTGTGACAA |
| | | SKAKGQPREPQVYTLPPS | AACTCACACATGCCCACCGTGCCCAGCACCTGAACT |
| | | REEMTKNQVSLTCLVKG | CCTGGGGGGACCGTCAGTCTTCCTCTTCCCCCCAAA |
| | | FYPSDIAVEWESNGQPEN | ACCCAAGGACACCCTCATGATCTCCCGGACCCCTGA |
| | | NYKTTPPVLDSDGSFFLY | GGTCACATGCGTGGTGGTGGACGTGAGCCACGAAG |
| | | SKLTVDKSRWQQGNVFS | ACCCTGAGGTCAAGTTCAACTGGTACGTGGACGGC |
| | | CSVMHEALHNHYTQKSL | GTGGAGGTGCATAATGCCAAGACAAAGCCGTGCGA |
| | | SLSPGK (SEQ ID NO: | GGAGCAGTACGGCAGCACGTACCGTTGCGTCAGCG |
| | | 525) | TCCTCACCGTCCTGCACCAGGACTGGCTGAATGGCA |
| | | | AGGAGTACAAGTGCAAGGTGTCCAACAAAGCCCTC |
| | | | CCAGCCCCCATCGAGAAAACCATCTCCAAAGCCAA |
| | | | AGGGCAGCCCCGAGAACCACAGGTGTACACCCTGC |
| | | | CCCCATCCCGGGAGGAGATGACCAAGAACCAGGTC |
| | | | AGCCTGACCTGCCTGGTCAAAGGCTTCTATCCCAGC |
| | | | GACATCGCCGTGGAGTGGGAGAGCAATGGGCAGCC |
| | | | GGAGAACAACTACAAGACCACGCCTCCCGTGCTGG |
| | | | ACTCCGACGGCTCCTTCTTCCTCTATAGCAAGCTCA |
| | | | CCGTGGACAAGAGCAGGTGGCAGCAGGGGAACGTC |
| | | | TTCTCATGCTCCGTGATGCATGAGGCTCTGCACAAC |
| | | | CACTACACGCAGAAGAGCCTCTCCCTGTCTCCGGGT |
| | | | AAA (SEQ ID NO: 558) |
| iPS:421091 | HC-08 | QVQLVESGGGVVQPGRS | CAGGTGCAGCTGGTGGAGTCTGGGGGAGGCGTGGT |
| | | LRLSCAASGFTFSRFAMH | CCAGCCTGGGAGGTCCCTGCGACTCTCCTGTGCAGC |
| | | WVRQAPGKGLEWVAVIS | CTCTGGATTCACCTTCAGTAGATTTGCCATGCACTG |
| | | YNGRNKYYARSVKGRFT | GGTCCGCCAGGCTCCAGGCAAGGGGCTGGAGTGGG |
| | | ISRDNSKNTLYLQMNSLR | TGGCAGTTATATCATATAACGGACGCAATAAATACT |
| | | AEDTALFYCARGYDILTG | ATGCACGCTCCGTGAAGGGCCGGTTCACCATCTCCA |
| | | YPDYWGQGTLVTVSSAS | GAGACAATTCCAAGAACACCCTGTATCTGCAAATG |
| | | TKGPSVFPLAPSSKSTSGG | AACAGCCTGAGAGCTGAGGACACGGCTCTGTTTTA |
| | | TAALGCLVKDYFPEPVTV | CTGTGCGAGAGGATACGATATCTTGACTGGTTACCC |
| | | SWNSGALTSGVHTFPAVL | CGACTACTGGGGCCAGGGAACCCTGGTCACCGTCT |
| | | QSSGLYSLSSVVTVPSSSL | CTAGTGCCTCCACCAAGGGCCCATCGGTCTTCCCCC |
| | | GTQTYICNVNHKPSNTKV | TGGCACCCTCCTCCAAGAGCACCTCTGGGGGCACA |
| | | DKKVEPKSCDKTHTCPPC | GCGGCCCTGGGCTGCCTGGTCAAGGACTACTTCCCC |
| | | PAPELLGGPSVFLFPPKPK | GAACCGGTGACGGTGTCGTGGAACTCAGGCGCCCT |
| | | DTLMISRTPEVTCVVVDV | GACCAGCGGCGTGCACACCTTCCCGGCTGTCCTACA |
| | | SHEDPEVKFNWYVDGVE | GTCCTCAGGACTCTACTCCCTCAGCAGCGTGGTGAC |
| | | VHNAKTKPCEEQYGSTY | CGTGCCCTCCAGCAGCTTGGGCACCCAGACCTACAT |
| | | RCVSVLTVLHQDWLNGK | CTGCAACGTGAATCACAAGCCCAGCAACACCAAGG |
| | | EYKCKVSNKALPAPIEKTI | TGGACAAGAAAGTTGAGCCCAAATCTTGTGACAAA |
| | | SKAKGQPREPQVYTLPPS | ACTCACACATGCCCACCGTGCCCAGCACCTGAACTC |
| | | REEMTKNQVSLTCLVKG | CTGGGGGGACCGTCAGTCTTCCTCTTCCCCCCAAAA |
| | | FYPSDIAVEWESNGQPEN | CCCAAGGACACCCTCATGATCTCCCGGACCCCTGAG |
| | | NYKTTPPVLDSDGSFFLY | GTCACATGCGTGGTGGTGGACGTGAGCCACGAAGA |
| | | SKLTVDKSRWQQGNVFS | CCCTGAGGTCAAGTTCAACTGGTACGTGGACGGCG |
| | | CSVMHEALHNHYTQKSL | TGGAGGTGCATAATGCCAAGACAAAGCCGTGCGAG |
| | | SLSPGK (SEQ ID NO: | GAGCAGTACGGCAGCACGTACCGTTGCGTCAGCGT |
| | | 526) | CCTCACCGTCCTGCACCAGGACTGGCTGAATGGCA |
| | | | AGGAGTACAAGTGCAAGGTGTCCAACAAAGCCCTC |
| | | | CCAGCCCCCATCGAGAAAACCATCTCCAAAGCCAA |
| | | | AGGGCAGCCCCGAGAACCACAGGTGTACACCCTGC |
| | | | CCCCATCCCGGGAGGAGATGACCAAGAACCAGGTC |
| | | | AGCCTGACCTGCCTGGTCAAAGGCTTCTATCCCAGC |
| | | | GACATCGCCGTGGAGTGGGAGAGCAATGGGCAGCC |
| | | | GGAGAACAACTACAAGACCACGCCTCCCGTGCTGG |
| | | | ACTCCGACGGCTCCTTCTTCCTCTATAGCAAGCTCA |
| | | | CCGTGGACAAGAGCAGGTGGCAGCAGGGGAACGTC |
| | | | TTCTCATGCTCCGTGATGCATGAGGCTCTGCACAAC |
| | | | CACTACACGCAGAAGAGCCTCTCCCTGTCTCCGGGT |
| | | | AAA (SEQ ID NO: 559) |
| iPS:421051 | HC-05 | QVQLVESGGGVVQPGRS | CAGGTGCAGCTGGTGGAGTCTGGGGGAGGCGTGGT |
| | | LRLSCAASGFTFSRFAMH | CCAGCCTGGGAGGTCCCTGCGACTCTCCTGTGCAGC |
| | | WVRQAPGKGLEWVAVIS | CTCTGGATTCACCTTCAGTAGATTTGCCATGCACTG |
| | | YQGNNKYYARSVKGRFT | GGTCCGCCAGGCTCCAGGCAAGGGGCTGGAGTGGG |
| | | ISRDNSKNTLYLQMNSLR | TGGCAGTTATATCATATCAGGGAAACAATAAATAC |
| | | AEDTALFYCARGYDVLT | TATGCACGCTCCGTGAAGGGCCGGTTCACCATCTCC |
| | | GYPDYWGQGTLVTVSSA | AGAGACAATTCCAAGAACACCCTGTATCTGCAAAT |
| | | STKGPSVFPLAPSSKSTSG | GAACAGCCTGAGAGCTGAGGACACGGCTCTGTTTT |
| | | GTAALGCLVKDYFPEPVT | ACTGTGCGAGAGGATACGATGTTTTGACTGGTTACC |
| | | VSWNSGALTSGVHTFPA | CCGACTACTGGGGCCAGGGAACCCTGGTCACCGTC |
| | | VLQSSGLYSLSSVVTVPSS | TCTAGTGCCTCCACCAAGGGCCCATCGGTCTTCCCC |
| | | SLGTQTYICNVNHKPSNT | CTGGCACCCTCCTCCAAGAGCACCTCTGGGGGCAC |
| | | KVDKKVEPKSCDKTHTC | AGCGGCCCTGGGCTGCCTGGTCAAGGACTACTTCCC |
| | | PPCPAPELLGGPSVFLFPP | CGAACCGGTGACGGTGTCGTGGAACTCAGGCGCCC |
| | | KPKDTLMISRTPEVTCVV | TGACCAGCGGCGTGCACACCTTCCCGGCTGTCCTAC |
| | | VDVSHEDPEVKFNWYVD | AGTCCTCAGGACTCTACTCCCTCAGCAGCGTGGTGA |
| | | GVEVHNAKTKPCEEQYG | CCGTGCCCTCCAGCAGCTTGGGCACCCAGACCTACA |
| | | STYRCVSVLTVLHQDWL | TCTGCAACGTGAATCACAAGCCCAGCAACACCAAG |
| | | NGKEYKCKVSNKALPAPI | GTGGACAAGAAAGTTGAGCCCAAATCTTGTGACAA |
| | | EKTISKAKGQPREPQVYT | AACTCACACATGCCCACCGTGCCCAGCACCTGAACT |
| | | LPPSREEMTKNQVSLTCL | CCTGGGGGGACCGTCAGTCTTCCTCTTCCCCCCAAA |
| | | VKGFYPSDIAVEWESNGQ | ACCCAAGGACACCCTCATGATCTCCCGGACCCCTGA |
| | | PENNYKTTPPVLDSDGSF | GGTCACATGCGTGGTGGTGGACGTGAGCCACGAAG |
| | | FLYSKLTVDKSRWQQGN | ACCCTGAGGTCAAGTTCAACTGGTACGTGGACGGC |
| | | VFSCSVMHEALHNHYTQ | GTGGAGGTGCATAATGCCAAGACAAAGCCGTGCGA |
| | | KSLSLSPGK (SEQ ID | GGAGCAGTACGGCAGCACGTACCGTTGCGTCAGCG |
| | | NO: 523) | TCCTCACCGTCCTGCACCAGGACTGGCTGAATGGCA |
| | | | AGGAGTACAAGTGCAAGGTGTCCAACAAAGCCCTC |
| | | | CCAGCCCCCATCGAGAAAACCATCTCCAAAGCCAA |
| | | | AGGGCAGCCCCGAGAACCACAGGTGTACACCCTGC |
| | | | CCCCATCCCGGGAGGAGATGACCAAGAACCAGGTC |
| | | | AGCCTGACCTGCCTGGTCAAAGGCTTCTATCCCAGC |
| | | | GACATCGCCGTGGAGTGGGAGAGCAATGGGCAGCC |
| | | | GGAGAACAACTACAAGACCACGCCTCCCGTGCTGG |
| | | | ACTCCGACGGCTCCTTCTTCCTCTATAGCAAGCTCA |
| | | | CCGTGGACAAGAGCAGGTGGCAGCAGGGGAACGTC |
| | | | TTCTCATGCTCCGTGATGCATGAGGCTCTGCACAAC |
| | | | CACTACACGCAGAAGAGCCTCTCCCTGTCTCCGGGT |
| | | | AAA (SEQ ID NO: 556) |
| iPS:480711 | HC-09 | QVQLVESGGGVVQPGRS | CAGGTGCAGCTGGTGGAGTCTGGGGGAGGCGTGG |
| | | LRLSCAASGFTFSRFAMH | TCCAGCCTGGGAGGTCCCTGCGACTCTCCTGTGCAG |
| | | WVRQAPGKGLEWVGVIN | CCTCTGGATTCACCTTCAGTAGATTTGCCATGCACT |
| | | YRGHGKYYAESVKGRFT | GGGTCCGCCAGGCTCCAGGCAAGGGGCTGGAGTGG |
| | | VSRDNSKNTLYLQMNSL | GTGGGTGTTATCAACTATCGTGGACATGGTAAATAC |
| | | RAEDTALFYCARGYDVL | TATGCAGAGTCCGTGAAGGGCCGGTTCACCGTGTCC |
| | | TGYPDYWGQGTLVTVSS | AGAGACAATTCCAAGAACACCCTGTATCTGCAAAT |
| | | ASTKGPSVFPLAPSSKSTS | GAACAGCCTGAGAGCTGAGGACACGGCTCTGTTTT |
| | | GGTAALGCLVKDYFPEP | ACTGTGCGAGAGGATACGATGTTTTGACTGGTTACC |
| | | VTVSWNSGALTSGVHTFP | CCGACTACTGGGGCCAGGGAACCCTGGTCACCGTG |
| | | AVLQSSGLYSLSSVVTVP | TCTAGTGCCTCCACCAAGGGCCCATCGGTCTTCCCC |
| | | SSSLGTQTYICNVNHKPS | CTGGCACCCTCCTCCAAGAGCACCTCTGGGGGCAC |
| | | NTKVDKKVEPKSCDKTH | AGCGGCCCTGGGCTGCCTGGTCAAGGACTACTTCCC |
| | | TCPPCPAPELLGGPSVFLF | CGAACCGGTGACGGTGTCGTGGAACTCAGGCGCCC |
| | | PPKPKDTLMISRTPEVTC | TGACCAGCGGCGTGCACACCTTCCCGGCTGTCCTAC |
| | | VVVDVSHEDPEVKFNWY | AGTCCTCAGGACTCTACTCCCTCAGCAGCGTGGTGA |
| | | VDGVEVHNAKTKPCEEQ | CCGTGCCCTCCAGCAGCTTGGGCACCCAGACCTACA |
| | | YGSTYRCVSVLTVLHQD | TCTGCAACGTGAATCACAAGCCCAGCAACACCAAG |
| | | WLNGKEYKCKVSNKALP | GTGGACAAGAAAGTTGAGCCCAAATCTTGTGACAA |
| | | APIEKTISKAKGQPREPQV | AACTCACACATGCCCACCGTGCCCAGCACCTGAACT |
| | | YTLPPSREEMTKNQVSLT | CCTGGGGGGACCGTCAGTCTTCCTCTTCCCCCCAAA |
| | | CLVKGFYPSDIAVEWESN | ACCCAAGGACACCCTCATGATCTCCCGGACCCCTGA |
| | | GQPENNYKTTPPVLDSDG | GGTCACATGCGTGGTGGTGGACGTGAGCCACGAAG |
| | | SFFLYSKLTVDKSRWQQ | ACCCTGAGGTCAAGTTCAACTGGTACGTGGACGGC |
| | | GNVFSCSVMHEALHNHY | GTGGAGGTGCATAATGCCAAGACAAAGCCGTGCGA |
| | | TQKSLSLSPGK (SEQ ID | GGAGCAGTACGGCAGCACGTACCGTTGCGTCAGCG |
| | | NO: 527) | TCCTCACCGTCCTGCACCAGGACTGGCTGAATGGCA |
| | | | AGGAGTACAAGTGCAAGGTGTCCAACAAAGCCCTC |
| | | | CCAGCCCCCATCGAGAAAACCATCTCCAAAGCCAA |
| | | | AGGGCAGCCCCGAGAACCACAGGTGTACACCCTGC |
| | | | CCCCATCCCGGGAGGAGATGACCAAGAACCAGGTC |
| | | | AGCCTGACCTGCCTGGTCAAAGGCTTCTATCCCAGC |
| | | | GACATCGCCGTGGAGTGGGAGAGCAATGGGCAGCC |
| | | | GGAGAACAACTACAAGACCACGCCTCCCGTGCTGG |
| | | | ACTCCGACGGCTCCTTCTTCCTCTATAGCAAGCTCA |
| | | | CCGTGGACAAGAGCAGGTGGCAGCAGGGGAACGTC |
| | | | TTCTCATGCTCCGTGATGCATGAGGCTCTGCACAAC |
| | | | CACTACACGCAGAAGAGCCTCTCCCTGTCTCCGGGT |
| | | | AAA (SEQ ID NO: 560) |
| iPS:480706 | HC-10 | QVQLVESGGGVVQPGRS | CAGGTGCAGCTGGTGGAGTCTGGGGGAGGCGTGGT |
| | | LRLSCAASGFTFSRFAMH | CCAGCCTGGGAGGTCCCTGCGACTCTCCTGTGCAGC |
| | | WVRQAPGKGLEWVGVIS | CTCTGGATTCACCTTCAGTAGATTTGCCATGCACTG |
| | | FSGGSKYYAESVKGRFTL | GGTCCGCCAGGCTCCAGGCAAGGGGCTGGAGTGGG |
| | | SRDNSKNTLYLQMNSLR | TGGGTGTTATATCTTTTTCTGGAGGTTCTAAATACT |
| | | AEDTALFYCARGYDVLT | ATGCAGAGTCCGTGAAGGGCCGGTTCACCTTGTCCA |
| | | GYPDYWGQGTLVTVSSA | GAGACAATTCCAAGAACACCCTGTATCTGCAAATG |
| | | STKGPSVFPLAPSSKSTSG | AACAGCCTGAGAGCTGAGGACACGGCTCTGTTTTA |
| | | GTAALGCLVKDYFPEPVT | CTGTGCGAGAGGATACGATGTTTTGACTGGTTACCC |
| | | VSWNSGALTSGVHTFPA | CGACTACTGGGGCCAGGGAACCCTGGTCACCGTGT |
| | | VLQSSGLYSLSSVVTVPSS | CTAGTGCCTCCACCAAGGGCCCATCGGTCTTCCCCC |
| | | SLGTQTYICNVNHKPSNT | TGGCACCCTCCTCCAAGAGCACCTCTGGGGGCACA |
| | | KVDKKVEPKSCDKTHTC | GCGGCCCTGGGCTGCCTGGTCAAGGACTACTTCCCC |
| | | PPCPAPELLGGPSVFLFPP | GAACCGGTGACGGTGTCGTGGAACTCAGGCGCCCT |
| | | KPKDTLMISRTPEVTCVV | GACCAGCGGCGTGCACACCTTCCCGGCTGTCCTACA |
| | | VDVSHEDPEVKFNWYVD | GTCCTCAGGACTCTACTCCCTCAGCAGCGTGGTGAC |
| | | GVEVHNAKTKPCEEQYG | CGTGCCCTCCAGCAGCTTGGGCACCCAGACCTACAT |
| | | STYRCVSVLTVLHQDWL | CTGCAACGTGAATCACAAGCCCAGCAACACCAAGG |
| | | NGKEYKCKVSNKALPAPI | TGGACAAGAAAGTTGAGCCCAAATCTTGTGACAAA |
| | | EKTISKAKGQPREPQVYT | ACTCACACATGCCCACCGTGCCCAGCACCTGAACTC |
| | | LPPSREEMTKNQVSLTCL | CTGGGGGGACCGTCAGTCTTCCTCTTCCCCCCAAAA |
| | | VKGFYPSDIAVEWESNGQ | CCCAAGGACACCCTCATGATCTCCCGGACCCCTGAG |
| | | PENNYKTTPPVLDSDGSF | GTCACATGCGTGGTGGTGGACGTGAGCCACGAAGA |
| | | FLYSKLTVDKSRWQQGN | CCCTGAGGTCAAGTTCAACTGGTACGTGGACGGCG |
| | | VFSCSVMHEALHNHYTQ | TGGAGGTGCATAATGCCAAGACAAAGCCGTGCGAG |
| | | KSLSLSPGK (SEQ ID | GAGCAGTACGGCAGCACGTACCGTTGCGTCAGCGT |
| | | NO: 528) | CCTCACCGTCCTGCACCAGGACTGGCTGAATGGCA |
| | | | AGGAGTACAAGTGCAAGGTGTCCAACAAAGCCCTC |
| | | | CCAGCCCCCATCGAGAAAACCATCTCCAAAGCCAA |
| | | | AGGGCAGCCCCGAGAACCACAGGTGTACACCCTGC |
| | | | CCCCATCCCGGGAGGAGATGACCAAGAACCAGGTC |
| | | | AGCCTGACCTGCCTGGTCAAAGGCTTCTATCCCAGC |
| | | | GACATCGCCGTGGAGTGGGAGAGCAATGGGCAGCC |
| | | | GGAGAACAACTACAAGACCACGCCTCCCGTGCTGG |
| | | | ACTCCGACGGCTCCTTCTTCCTCTATAGCAAGCTCA |
| | | | CCGTGGACAAGAGCAGGTGGCAGCAGGGGAACGTC |
| | | | TTCTCATGCTCCGTGATGCATGAGGCTCTGCACAAC |
| | | | CACTACACGCAGAAGAGCCTCTCCCTGTCTCCGGGT |
| | | | AAA (SEQ ID NO: 561) |
| iPS:480713 | HC-11 | QVQLVESGGGVVQPGRS | CAGGTGCAGCTGGTGGAGTCTGGGGGAGGCGTGGT |
| | | LRLSCAASGFTFSRFAMH | CCAGCCTGGGAGGTCCCTGCGACTCTCCTGTGCAGC |
| | | WVRQAPGKGLEWVGVIS | CTCTGGATTCACCTTCAGTAGATTTGCCATGCACTG |
| | | YTGQFKYYAESVKGRFT | GGTCCGCCAGGCTCCAGGCAAGGGGCTGGAGTGGG |
| | | VSRDNSKNTLYLQMNSL | TGGGTGTTATCTCTTATACTGGACAGTTCAAATACT |
| | | RAEDTALFYCARGYDVL | ATGCAGAGTCCGTGAAGGGCCGGTTCACCGTGTCC |
| | | TGYPDYWGQGTLVTVSS | AGAGACAATTCCAAGAACACCCTGTATCTGCAAAT |
| | | ASTKGPSVFPLAPSSKSTS | GAACAGCCTGAGAGCTGAGGACACGGCTCTGTTTT |
| | | GGTAALGCLVKDYFPEP | ACTGTGCGAGAGGATACGATGTTTTGACTGGTTACC |
| | | VTVSWNSGALTSGVHTFP | CCGACTACTGGGGCCAGGGAACCCTGGTCACCGTG |
| | | AVLQSSGLYSLSSVVTVP | TCTAGTGCCTCCACCAAGGGCCCATCGGTCTTCCCC |
| | | SSSLGTQTYICNVNHKPS | CTGGCACCCTCCTCCAAGAGCACCTCTGGGGGCAC |
| | | NTKVDKKVEPKSCDKTH | AGCGGCCCTGGGCTGCCTGGTCAAGGACTACTTCCC |
| | | TCPPCPAPELLGGPSVFLF | CGAACCGGTGACGGTGTCGTGGAACTCAGGCGCCC |
| | | PPKPKDTLMISRTPEVTC | TGACCAGCGGCGTGCACACCTTCCCGGCTGTCCTAC |
| | | VVVDVSHEDPEVKFNWY | AGTCCTCAGGACTCTACTCCCTCAGCAGCGTGGTGA |
| | | VDGVEVHNAKTKPCEEQ | CCGTGCCCTCCAGCAGCTTGGGCACCCAGACCTACA |
| | | YGSTYRCVSVLTVLHQD | TCTGCAACGTGAATCACAAGCCCAGCAACACCAAG |
| | | WLNGKEYKCKVSNKALP | GTGGACAAGAAAGTTGAGCCCAAATCTTGTGACAA |
| | | APIEKTISKAKGQPREPQV | AACTCACACATGCCCACCGTGCCCAGCACCTGAACT |
| | | YTLPPSREEMTKNQVSLT | CCTGGGGGGACCGTCAGTCTTCCTCTTCCCCCCAAA |
| | | CLVKGFYPSDIAVEWESN | ACCCAAGGACACCCTCATGATCTCCCGGACCCCTGA |
| | | GQPENNYKTTPPVLDSDG | GGTCACATGCGTGGTGGTGGACGTGAGCCACGAAG |
| | | SFFLYSKLTVDKSRWQQ | ACCCTGAGGTCAAGTTCAACTGGTACGTGGACGGC |
| | | GNVFSCSVMHEALHNHY | GTGGAGGTGCATAATGCCAAGACAAAGCCGTGCGA |
| | | TQKSLSLSPGK (SEQ ID | GGAGCAGTACGGCAGCACGTACCGTTGCGTCAGCG |
| | | NO: 529) | TCCTCACCGTCCTGCACCAGGACTGGCTGAATGGCA |
| | | | AGGAGTACAAGTGCAAGGTGTCCAACAAAGCCCTC |
| | | | CCAGCCCCCATCGAGAAAACCATCTCCAAAGCCAA |
| | | | AGGGCAGCCCCGAGAACCACAGGTGTACACCCTGC |
| | | | CCCCATCCCGGGAGGAGATGACCAAGAACCAGGTC |
| | | | AGCCTGACCTGCCTGGTCAAAGGCTTCTATCCCAGC |
| | | | GACATCGCCGTGGAGTGGGAGAGCAATGGGCAGCC |
| | | | GGAGAACAACTACAAGACCACGCCTCCCGTGCTGG |
| | | | ACTCCGACGGCTCCTTCTTCCTCTATAGCAAGCTCA |
| | | | CCGTGGACAAGAGCAGGTGGCAGCAGGGGAACGTC |
| | | | TTCTCATGCTCCGTGATGCATGAGGCTCTGCACAAC |
| | | | CACTACACGCAGAAGAGCCTCTCCCTGTCTCCGGGT |
| | | | AAA (SEQ ID NO: 562) |

19H8 variants

| | | | |
|---|---|---|---|
| 19H8 | HC-12 | QVQLQQSGPGLVKPSQTL | CAGGTACAGCTGCAGCAGTCAGGTCCAGGACTGGT |
| | | SLTCAISGDSVSSNSATW | GAAGCCCTCGCAGACCCTCTCACTCACCTGTGCCAT |
| | | NWIRQSPSRGLEWLGRTY | CTCCGGGGACAGTGTCTCTAGCAACAGTGCTACTTG |
| | | YRSKWSNHYAVSVKSRIT | GAACTGGATCAGGCAGTCCCCATCGAGAGGCCTTG |
| | | INPDTSKSQFSLQLNSVTP | AGTGGCTGGGAAGGACATATTACAGGTCCAAGTGG |
| | | EDTAVYYCARGTWKQL | TCTAATCATTATGCAGTATCTGTGAAAAGTCGAATC |
| | | WFLDHWGQGTLVTVSSA | ACCATCAACCCCGACACGTCCAAGAGCCAGTTCTCC |
| | | STKGPSVFPLAPSSKSTSG | CTGCAGCTGAACTCTGTGACTCCCGAGGACACGGCT |
| | | GTAALGCLVKDYFPEPVT | GTGTATTACTGTGCAAGAGGAACGTGGAAACAGCT |
| | | VSWNSGALTSGVHTFPA | ATGGTTCCTTGACCACTGGGGCCAGGGAACCCTGGT |
| | | VLQSSGLYSLSSVVTVPSS | CACCGTGTCTAGTGCCTCCACCAAGGGCCCATCGGT |
| | | SLGTQTYICNVNHKPSNT | CTTCCCCCTGGCACCCTCCTCCAAGAGCACCTCTGG |
| | | KVDKKVEPKSCDKTHTC | GGGCACAGCGGCCCTGGGCTGCCTGGTCAAGGACT |
| | | PPCPAPELLGGPSVFLFPP | ACTTCCCCGAACCGGTGACGGTGTCGTGGAACTCA |
| | | KPKDTLMISRTPEVTCVV | GGCGCCCTGACCAGCGGCGTGCACACCTTCCCGGCT |
| | | VDVSHEDPEVKFNWYVD | GTCCTACAGTCCTCAGGACTCTACTCCCTCAGCAGC |
| | | GVEVHNAKTKPCEEQYG | GTGGTGACCGTGCCCTCCAGCAGCTTGGGCACCCA |
| | | STYRCVSVLTVLHQDWL | GACCTACATCTGCAACGTGAATCACAAGCCCAGCA |
| | | NGKEYKCKVSNKALPAPI | ACACCAAGGTGGACAAGAAAGTTGAGCCCAAATCT |
| | | EKTISKAKGQPREPQVYT | TGTGACAAAACTCACACATGCCCACCGTGCCCAGC |
| | | LPPSREEMTKNQVSLTCL | ACCTGAACTCCTGGGGGGACCGTCAGTCTTCCTCTT |
| | | VKGFYPSDIAVEWESNGQ | CCCCCCAAAACCCAAGGACACCCTCATGATCTCCCG |
| | | PENNYKTTPPVLDSDGSF | GACCCCTGAGGTCACATGCGTGGTGGTGGACGTGA |
| | | FLYSKLTVDKSRWQQGN | GCCACGAAGACCCTGAGGTCAAGTTCAACTGGTAC |
| | | VFSCSVMHEALHNHYTQ | GTGGACGGCGTGGAGGTGCATAATGCCAAGACAAA |
| | | KSLSLSPGK (SEQ ID | GCCGTGCGAGGAGCAGTACGGCAGCACGTACCGTT |
| | | NO: 530) | GCGTCAGCGTCCTCACCGTCCTGCACCAGGACTGGC |
| | | | TGAATGGCAAGGAGTACAAGTGCAAGGTGTCCAAC |
| | | | AAAGCCCTCCCAGCCCCCATCGAGAAAACCATCTC |
| | | | CAAAGCCAAAGGGCAGCCCCGAGAACCACAGGTGT |
| | | | ACACCCTGCCCCCATCCCGGGAGGAGATGACCAAG |
| | | | AACCAGGTCAGCCTGACCTGCCTGGTCAAAGGCTTC |
| | | | TATCCCAGCGACATCGCCGTGGAGTGGGAGAGCAA |
| | | | TGGGCAGCCGGAGAACAACTACAAGACCACGCCTC |
| | | | CCGTGCTGGACTCCGACGGCTCCTTCTTCCTCTATA |
| | | | GCAAGCTCACCGTGGACAAGAGCAGGTGGCAGCAG |
| | | | GGGAACGTCTTCTCATGCTCCGTGATGCATGAGGCT |
| | | | CTGCACAACCACTACACGCAGAAGAGCCTCTCCCT |
| | | | GTCTCCGGGCAAA (SEQ ID NO: 563) |
| iPS:448730 | HC-13 | QVQLQQSGPGLVKPSQTL | CAGGTACAGCTGCAGCAGTCAGGTCCAGGACTGGT |
| | | SLTCAISGDSVSNRLATW | GAAGCCCTCGCAGACCCTCTCACTCACCTGTGCCAT |
| | | NWIRQSPSRGLEWLGRTY | CTCCGGGGACAGTGTCTCTAACCGTCTGGCTACTTG |
| | | YRGKWKNHYAVSVKSRI | GAACTGGATCAGGCAGTCCCCATCGAGAGGCCTTG |
| | | TINPDTSKSQFSLQLNSVT | AGTGGCTGGGAAGGACATACTACAGGGGTAAATGG |
| | | PEDTAVYYCARGVWIGN | AAAAATCATTATGCAGTATCTGTGAAAAGTCGAAT |
| | | WFLDHWGQGTLVTVSSA | AACCATCAACCCCGACACGTCCAAGAGCCAGTTCT |
| | | STKGPSVFPLAPSSKSTSG | CCCTGCAGCTGAACTCTGTGACTCCCGAGGACACG |
| | | GTAALGCLVKDYFPEPVT | GCTGTGTATTACTGTGCAAGAGGAGTTTGGATCGGT |
| | | VSWNSGALTSGVHTFPA | AACTGGTTCCTGGACCACTGGGGCCAGGGAACCCT |
| | | VLQSSGLYSLSSVVTVPSS | GGTCACCGTGTCCTCAGCCTCCACCAAGGGCCCATC |
| | | SLGTQTYICNVNHKPSNT | GGTCTTCCCCCTGGCACCCTCCTCCAAGAGCACCTC |
| | | KVDKKVEPKSCDKTHTC | TGGGGGCACAGCGGCCCTGGGCTGCCTGGTCAAGG |
| | | PPCPAPELLGGPSVFLFPP | ACTACTTCCCCGAACCGGTGACGGTGTCGTGGAACT |
| | | KPKDTLMISRTPEVTCVV | CAGGCGCCCTGACCAGCGGCGTGCACACCTTCCCG |
| | | VDVSHEDPEVKFNWYVD | GCTGTCCTACAGTCCTCAGGACTCTACTCCCTCAGC |
| | | GVEVHNAKTKPCEEQYG | AGCGTGGTGACCGTGCCCTCCAGCAGCTTGGGCAC |
| | | STYRCVSVLTVLHQDWL | CCAGACCTACATCTGCAACGTGAATCACAAGCCCA |
| | | NGKEYKCKVSNKALPAPI | GCAACACCAAGGTGGACAAGAAAGTTGAGCCCAAA |
| | | EKTISKAKGQPREPQVYT | TCTTGTGACAAAACTCACACATGCCCACCGTGCCCA |
| | | LPPSREEMTKNQVSLTCL | GCACCTGAACTCCTGGGGGGACCGTCAGTCTTCCTC |
| | | VKGFYPSDIAVEWESNGQ | TTCCCCCCAAAACCCAAGGACACCCTCATGATCTCC |
| | | PENNYKTTPPVLDSDGSF | CGGACCCCTGAGGTCACATGCGTGGTGGTGGACGT |
| | | FLYSKLTVDKSRWQQGN | GAGCCACGAAGACCCTGAGGTCAAGTTCAACTGGT |
| | | VFSCSVMHEALHNHYTQ | ACGTGGACGGCGTGGAGGTGCATAATGCCAAGACA |
| | | KSLSLSPGK (SEQ ID | AAGCCGTGCGAGGAGCAGTACGGCAGCACGTACCG |
| | | NO: 531) | TTGCGTCAGCGTCCTCACCGTCCTGCACCAGGACTG |
| | | | GCTGAATGGCAAGGAGTACAAGTGCAAGGTGTCCA |
| | | | ACAAAGCCCTCCCAGCCCCCATCGAGAAAACCATC |
| | | | TCCAAAGCCAAAGGGCAGCCCCGAGAACCACAGGT |
| | | | GTACACCCTGCCCCCATCCCGGGAGGAGATGACCA |
| | | | AGAACCAGGTCAGCCTGACCTGCCTGGTCAAAGGC |
| | | | TTCTATCCCAGCGACATCGCCGTGGAGTGGGAGAG |
| | | | CAATGGGCAGCCGGAGAACAACTACAAGACCACGC |
| | | | CTCCCGTGCTGGACTCCGACGGCTCCTTCTTCCTCT |
| | | | ATAGCAAGCTCACCGTGGACAAGAGCAGGTGGCAG |
| | | | CAGGGGAACGTCTTCTCATGCTCCGTGATGCATGAG |
| | | | GCTCTGCACAACCACTACACGCAGAAGAGCCTCTC |
| | | | CCTGTCTCCGGGTAAA (SEQ ID NO: 564) |
| iPS:448195 | HC-14 | QVQLQQSGPGLVKPSQTL | CAGGTACAGCTGCAGCAGTCAGGTCCAGGACTGGT |
| | | SLTCAISGDSVSNKQATW | GAAGCCCTCGCAGACCCTCTCACTCACCTGTGCCAT |
| | | NWIRQSPSRGLEWLGRTY | CTCCGGGGACAGTGTCTCTAACAAACAGGCTACTTG |
| | | YRGKWKNHYAVSVKSRI | GAACTGGATCAGGCAGTCCCCATCGAGAGGCCTTG |
| | | TINPDTSKSQFSLQLNSVT | AGTGGCTGGGAAGGACATACTACAGGGGTAAATGG |
| | | PEDTAVYYCARGMWNQ | AAAAATCATTATGCAGTATCTGTGAAAAGTCGAAT |
| | | NWFLDHWGQGTLVTVSS | AACCATCAACCCCGACACGTCCAAGAGCCAGTTCT |
| | | ASTKGPSVFPLAPSSKSTS | CCCTGCAGCTGAACTCTGTGACTCCCGAGGACACG |
| | | GGTAALGCLVKDYFPEP | GCTGTGTATTACTGTGCAAGAGGAATGTGGAACCA |
| | | VTVSWNSGALTSGVHTFP | GAACTGGTTCCTGGACCACTGGGGCCAGGGAACCC |
| | | AVLQSSGLYSLSSVVTVP | TGGTCACCGTGTCCTCAGCCTCCACCAAGGGCCCAT |
| | | SSSLGTQTYICNVNHKPS | CGGTCTTCCCCCTGGCACCCTCCTCCAAGAGCACCT |
| | | NTKVDKKVEPKSCDKTH | CTGGGGGCACAGCGGCCCTGGGCTGCCTGGTCAAG |
| | | TCPPCPAPELLGGPSVFLF | GACTACTTCCCCGAACCGGTGACGGTGTCGTGGAA |
| | | PPKPKDTLMISRTPEVTC | CTCAGGCGCCCTGACCAGCGGCGTGCACACCTTCCC |
| | | VVVDVSHEDPEVKFNWY | GGCTGTCCTACAGTCCTCAGGACTCTACTCCCTCAG |
| | | VDGVEVHNAKTKPCEEQ | CAGCGTGGTGACCGTGCCCTCCAGCAGCTTGGGCA |
| | | YGSTYRCVSVLTVLHQD | CCCAGACCTACATCTGCAACGTGAATCACAAGCCC |
| | | WLNGKEYKCKVSNKALP | AGCAACACCAAGGTGGACAAGAAAGTTGAGCCCAA |
| | | APIEKTISKAKGQPREPQV | ATCTTGTGACAAAACTCACACATGCCCACCGTGCCC |
| | | YTLPPSREEMTKNQVSLT | AGCACCTGAACTCCTGGGGGGACCGTCAGTCTTCCT |
| | | CLVKGFYPSDIAVEWESN | CTTCCCCCCAAAACCCAAGGACACCCTCATGATCTC |
| | | GQPENNYKTTPPVLDSDG | CCGGACCCCTGAGGTCACATGCGTGGTGGTGGACG |
| | | SFFLYSKLTVDKSRWQQ | TGAGCCACGAAGACCCTGAGGTCAAGTTCAACTGG |
| | | GNVFSCSVMHEALHNHY | TACGTGGACGGCGTGGAGGTGCATAATGCCAAGAC |
| | | TQKSLSLSPGK (SEQ ID | AAAGCCGTGCGAGGAGCAGTACGGCAGCACGTACC |
| | | NO: 532) | GTTGCGTCAGCGTCCTCACCGTCCTGCACCAGGACT |
| | | | GGCTGAATGGCAAGGAGTACAAGTGCAAGGTGTCC |
| | | | AACAAAGCCCTCCCAGCCCCCATCGAGAAAACCAT |
| | | | CTCCAAAGCCAAAGGGCAGCCCCGAGAACCACAGG |
| | | | TGTACACCCTGCCCCCATCCCGGGAGGAGATGACC |
| | | | AAGAACCAGGTCAGCCTGACCTGCCTGGTCAAAGG |
| | | | CTTCTATCCCAGCGACATCGCCGTGGAGTGGGAGA |
| | | | GCAATGGGCAGCCGGAGAACAACTACAAGACCACG |
| | | | CCTCCCGTGCTGGACTCCGACGGCTCCTTCTTCCTCT |
| | | | ATAGCAAGCTCACCGTGGACAAGAGCAGGTGGCAG |
| | | | CAGGGGAACGTCTTCTCATGCTCCGTGATGCATGAG |
| | | | GCTCTGCACAACCACTACACGCAGAAGAGCCTCTC |
| | | | CCTGTCTCCGGGTAAA (SEQ ID NO: 565) |
| iPS:448788 | HC-15 | QVQLQQSGPGLVKPSQTL | CAGGTACAGCTGCAGCAGTCAGGTCCAGGACTGGT |
| | | SLTCAISGDSVSSRQATW | GAAGCCCTCGCAGACCCTCTCACTCACCTGTGCCAT |
| | | NWIRQSPSRGLEWLGRTY | CTCCGGGGACAGTGTCTCTTCTCGTCAGGCTACTTG |
| | | YRGKWKNHYAVSVKSRI | GAACTGGATCAGGCAGTCCCCATCGAGAGGCCTTG |
| | | TINPDTSKSQFSLQLNSVT | AGTGGCTGGGAAGGACATACTACAGGGGTAAATGG |
| | | PEDTAVYYCARGMWQG | AAAAATCATTATGCAGTATCTGTGAAAAGTCGAAT |
| | | NWFLDHWGQGTLVTVSS | AACCATCAACCCCGACACGTCCAAGAGCCAGTTCT |
| | | ASTKGPSVFPLAPSSKSTS | CCCTGCAGCTGAACTCTGTGACTCCCGAGGACACG |
| | | GGTAALGCLVKDYFPEP | GCTGTGTATTACTGTGCAAGAGGAATGTGGCAGGG |
| | | VTVSWNSGALTSGVHTFP | TAACTGGTTCCTGGACCACTGGGGCCAGGGAACCC |
| | | AVLQSSGLYSLSSVVTVP | TGGTCACCGTGTCCTCAGCCTCCACCAAGGGCCCAT |
| | | SSSLGTQTYICNVNHKPS | CGGTCTTCCCCCTGGCACCCTCCTCCAAGAGCACCT |
| | | NTKVDKKVEPKSCDKTH | CTGGGGGCACAGCGGCCCTGGGCTGCCTGGTCAAG |
| | | TCPPCPAPELLGGPSVFLF | GACTACTTCCCCGAACCGGTGACGGTGTCGTGGAA |
| | | PPKPKDTLMISRTPEVTC | CTCAGGCGCCCTGACCAGCGGCGTGCACACCTTCCC |
| | | VVVDVSHEDPEVKFNWY | GGCTGTCCTACAGTCCTCAGGACTCTACTCCCTCAG |
| | | VDGVEVHNAKTKPCEEQ | CAGCGTGGTGACCGTGCCCTCCAGCAGCTTGGGCA |
| | | YGSTYRCVSVLTVLHQD | CCCAGACCTACATCTGCAACGTGAATCACAAGCCC |
| | | WLNGKEYKCKVSNKALP | AGCAACACCAAGGTGGACAAGAAAGTTGAGCCCAA |
| | | APIEKTISKAKGQPREPQV | ATCTTGTGACAAAACTCACACATGCCCACCGTGCCC |
| | | YTLPPSREEMTKNQVSLT | AGCACCTGAACTCCTGGGGGGACCGTCAGTCTTCCT |
| | | CLVKGFYPSDIAVEWESN | CTTCCCCCCAAAACCCAAGGACACCCTCATGATCTC |
| | | GQPENNYKTTPPVLDSDG | CCGGACCCCTGAGGTCACATGCGTGGTGGTGGACG |
| | | SFFLYSKLTVDKSRWQQ | TGAGCCACGAAGACCCTGAGGTCAAGTTCAACTGG |
| | | GNVFSCSVMHEALHNHY | TACGTGGACGGCGTGGAGGTGCATAATGCCAAGAC |
| | | TQKSLSLSPGK (SEQ ID | AAAGCCGTGCGAGGAGCAGTACGGCAGCACGTACC |
| | | NO: 533) | GTTGCGTCAGCGTCCTCACCGTCCTGCACCAGGACT |
| | | | GGCTGAATGGCAAGGAGTACAAGTGCAAGGTGTCC |
| | | | AACAAAGCCCTCCCAGCCCCCATCGAGAAAACCAT |
| | | | CTCCAAAGCCAAAGGGCAGCCCCGAGAACCACAGG |
| | | | TGTACACCCTGCCCCCATCCCGGGAGGAGATGACC |
| | | | AAGAACCAGGTCAGCCTGACCTGCCTGGTCAAAGG |
| | | | CTTCTATCCCAGCGACATCGCCGTGGAGTGGGAGA |
| | | | GCAATGGGCAGCCGGAGAACAACTACAAGACCACG |
| | | | CCTCCCGTGCTGGACTCCGACGGCTCCTTCTTCCTCT |
| | | | ATAGCAAGCTCACCGTGGACAAGAGCAGGTGGCAG |
| | | | CAGGGGAACGTCTTCTCATGCTCCGTGATGCATGAG |
| | | | GCTCTGCACAACCACTACACGCAGAAGAGCCTCTC |
| | | | CCTGTCTCCGGGTAAA (SEQ ID NO: 566) |
| iPS:448901 | HC-16 | QVQLQQSGPGLVKPSQTL | CAGGTACAGCTGCAGCAGTCAGGTCCAGGACTGGT |
| | | SLTCAISGDSVSNRLATW | GAAGCCCTCGCAGACCCTCTCACTCACCTGTGCCAT |
| | | NWIRQSPSRGLEWLGRTY | CTCCGGGGACAGTGTCTCTAACCGTCTGGCTACTTG |
| | | YRGKWKNHYAVSVKSRI | GAACTGGATCAGGCAGTCCCCATCGAGAGGCCTTG |
| | | TINPDTSKSQFSLQLNSVT | AGTGGCTGGGAAGGACATACTACAGGGGTAAATGG |
| | | PEDTAVYYCARGRWEGD | AAAAATCATTATGCAGTATCTGTGAAAAGTCGAAT |
| | | WFFDHWGQGTLVTVSSA | AACCATCAACCCCGACACGTCCAAGAGCCAGTTCT |
| | | STKGPSVFPLAPSSKSTSG | CCCTGCAGCTGAACTCTGTGACTCCCGAGGACACG |
| | | GTAALGCLVKDYFPEPVT | GCTGTGTATTACTGTGCAAGAGGACGTTGGGAAGG |
| | | VSWNSGALTSGVHTFPA | TGACTGGTTCTTCGACCACTGGGGCCAGGGAACCCT |
| | | VLQSSGLYSLSSVVTVPSS | GGTCACCGTGTCCTCAGCCTCCACCAAGGGCCCATC |
| | | SLGTQTYICNVNHKPSNT | GGTCTTCCCCCTGGCACCCTCCTCCAAGAGCACCTC |
| | | KVDKKVEPKSCDKTHTC | TGGGGGCACAGCGGCCCTGGGCTGCCTGGTCAAGG |
| | | PPCPAPELLGGPSVFLFPP | ACTACTTCCCCGAACCGGTGACGGTGTCGTGGAACT |
| | | KPKDTLMISRTPEVTCVV | CAGGCGCCCTGACCAGCGGCGTGCACACCTTCCCG |
| | | VDVSHEDPEVKFNWYVD | GCTGTCCTACAGTCCTCAGGACTCTACTCCCTCAGC |
| | | GVEVHNAKTKPCEEQYG | AGCGTGGTGACCGTGCCCTCCAGCAGCTTGGGCAC |
| | | STYRCVSVLTVLHQDWL | CCAGACCTACATCTGCAACGTGAATCACAAGCCCA |
| | | NGKEYKCKVSNKALPAPI | GCAACACCAAGGTGGACAAGAAAGTTGAGCCCAAA |
| | | EKTISKAKGQPREPQVYT | TCTTGTGACAAAACTCACACATGCCCACCGTGCCCA |
| | | LPPSREEMTKNQVSLTCL | GCACCTGAACTCCTGGGGGGACCGTCAGTCTTCCTC |
| | | VKGFYPSDIAVEWESNGQ | TTCCCCCCAAAACCCAAGGACACCCTCATGATCTCC |
| | | PENNYKTTPPVLDSDGSF | CGGACCCCTGAGGTCACATGCGTGGTGGTGGACGT |
| | | FLYSKLTVDKSRWQQGN | GAGCCACGAAGACCCTGAGGTCAAGTTCAACTGGT |
| | | VFSCSVMHEALHNHYTQ | ACGTGGACGGCGTGGAGGTGCATAATGCCAAGACA |
| | | KSLSLSPGK (SEQ ID | AAGCCGTGCGAGGAGCAGTACGGCAGCACGTACCG |
| | | NO: 534) | TTGCGTCAGCGTCCTCACCGTCCTGCACCAGGACTG |
| | | | GCTGAATGGCAAGGAGTACAAGTGCAAGGTGTCCA |
| | | | ACAAAGCCCTCCCAGCCCCCATCGAGAAAACCATC |
| | | | TCCAAAGCCAAAGGGCAGCCCCGAGAACCACAGGT |
| | | | GTACACCCTGCCCCCATCCCGGGAGGAGATGACCA |
| | | | AGAACCAGGTCAGCCTGACCTGCCTGGTCAAAGGC |
| | | | TTCTATCCCAGCGACATCGCCGTGGAGTGGGAGAG |
| | | | CAATGGGCAGCCGGAGAACAACTACAAGACCACGC |
| | | | CTCCCGTGCTGGACTCCGACGGCTCCTTCTTCCTCT |
| | | | ATAGCAAGCTCACCGTGGACAAGAGCAGGTGGCAG |
| | | | CAGGGGAACGTCTTCTCATGCTCCGTGATGCATGAG |
| | | | GCTCTGCACAACCACTACACGCAGAAGAGCCTCTC |
| | | | CCTGTCTCCGGGTAAA (SEQ ID NO: 567) |
| iPS:448689 | HC-17 | QVQLQQSGPGLVKPSQTL | CAGGTACAGCTGCAGCAGTCAGGTCCAGGACTGGT |
| | | SLTCAISGDSVSNRLATW | GAAGCCCTCGCAGACCCTCTCACTCACCTGTGCCAT |
| | | NWIRQSPSRGLEWLGRTY | CTCCGGGGACAGTGTCTCTAACCGTCTGGCTACTTG |
| | | YRGKWKNHYAVSVKSRI | GAACTGGATCAGGCAGTCCCCATCGAGAGGCCTTG |
| | | TINPDTSKSQFSLQLNSVT | AGTGGCTGGGAAGGACATACTACAGGGGTAAATGG |
| | | PEDTAVYYCARGTWNQD | AAAAATCATTATGCAGTATCTGTGAAAAGTCGAAT |
| | | WFLDHWGQGTLVTVSSA | AACCATCAACCCCGACACGTCCAAGAGCCAGTTCT |
| | | STKGPSVFPLAPSSKSTSG | CCCTGCAGCTGAACTCTGTGACTCCCGAGGACACG |
| | | GTAALGCLVKDYFPEPVT | GCTGTGTATTACTGTGCAAGAGGAACTTGGAACCA |
| | | VSWNSGALTSGVHTFPA | GGACTGGTTCCTGGACCACTGGGGCCAGGGAACCC |
| | | VLQSSGLYSLSSVVTVPSS | TGGTCACCGTGTCCTCAGCCTCCACCAAGGGCCCAT |
| | | SLGTQTYICNVNHKPSNT | CGGTCTTCCCCCTGGCACCCTCCTCCAAGAGCACCT |
| | | KVDKKVEPKSCDKTHTC | CTGGGGGCACAGCGGCCCTGGGCTGCCTGGTCAAG |
| | | PPCPAPELLGGPSVFLFPP | GACTACTTCCCCGAACCGGTGACGGTGTCGTGGAA |
| | | KPKDTLMISRTPEVTCVV | CTCAGGCGCCCTGACCAGCGGCGTGCACACCTTCCC |
| | | VDVSHEDPEVKFNWYVD | GGCTGTCCTACAGTCCTCAGGACTCTACTCCCTCAG |
| | | GVEVHNAKTKPCEEQYG | CAGCGTGGTGACCGTGCCCTCCAGCAGCTTGGGCA |
| | | STYRCVSVLTVLHQDWL | CCCAGACCTACATCTGCAACGTGAATCACAAGCCC |
| | | NGKEYKCKVSNKALPAPI | AGCAACACCAAGGTGGACAAGAAAGTTGAGCCCAA |
| | | EKTISKAKGQPREPQVYT | ATCTTGTGACAAAACTCACACATGCCCACCGTGCCC |
| | | LPPSREEMTKNQVSLTCL | AGCACCTGAACTCCTGGGGGGACCGTCAGTCTTCCT |
| | | VKGFYPSDIAVEWESNGQ | CTTCCCCCCAAAACCCAAGGACACCCTCATGATCTC |
| | | PENNYKTTPPVLDSDGSF | CCGGACCCCTGAGGTCACATGCGTGGTGGTGGACG |
| | | FLYSKLTVDKSRWQQGN | TGAGCCACGAAGACCCTGAGGTCAAGTTCAACTGG |
| | | VFSCSVMHEALHNHYTQ | TACGTGGACGGCGTGGAGGTGCATAATGCCAAGAC |
| | | KSLSLSPGK (SEQ ID | AAAGCCGTGCGAGGAGCAGTACGGCAGCACGTACC |
| | | NO: 535) | GTTGCGTCAGCGTCCTCACCGTCCTGCACCAGGACT |
| | | | GGCTGAATGGCAAGGAGTACAAGTGCAAGGTGTCC |
| | | | AACAAAGCCCTCCCAGCCCCCATCGAGAAAACCAT |
| | | | CTCCAAAGCCAAAGGGCAGCCCCGAGAACCACAGG |
| | | | TGTACACCCTGCCCCCATCCCGGGAGGAGATGACC |
| | | | AAGAACCAGGTCAGCCTGACCTGCCTGGTCAAAGG |
| | | | CTTCTATCCCAGCGACATCGCCGTGGAGTGGGAGA |
| | | | GCAATGGGCAGCCGGAGAACAACTACAAGACCACG |
| | | | CCTCCCGTGCTGGACTCCGACGGCTCCTTCTTCCTCT |
| | | | ATAGCAAGCTCACCGTGGACAAGAGCAGGTGGCAG |
| | | | CAGGGGAACGTCTTCTCATGCTCCGTGATGCATGAG |
| | | | GCTCTGCACAACCACTACACGCAGAAGAGCCTCTC |
| | | | CCTGTCTCCGGGTAAA (SEQ ID NO: 568) |
| iPS:448202 | HC-17 | QVQLQQSGPGLVKPSQTL | CAGGTACAGCTGCAGCAGTCAGGTCCAGGACTGGT |
| | | SLTCAISGDSVSNRLATW | GAAGCCCTCGCAGACCCTCTCACTCACCTGTGCCAT |
| | | NWIRQSPSRGLEWLGRTY | CTCCGGGGACAGTGTCTCTAACCGTCTGGCTACTTG |
| | | YRGKWKNHYAVSVKSRI | GAACTGGATCAGGCAGTCCCCATCGAGAGGCCTTG |
| | | TINPDTSKSQFSLQLNSVT | AGTGGCTGGGAAGGACATACTACAGGGGTAAATGG |
| | | PEDTAVYYCARGTWNQD | AAAAATCATTATGCAGTATCTGTGAAAAGTCGAAT |
| | | WFLDHWGQGTLVTVSSA | AACCATCAACCCCGACACGTCCAAGAGCCAGTTCT |
| | | STKGPSVFPLAPSSKSTSG | CCCTGCAGCTGAACTCTGTGACTCCCGAGGACACG |
| | | GTAALGCLVKDYFPEPVT | GCTGTGTATTACTGTGCAAGAGGAACTTGGAACCA |
| | | VSWNSGALTSGVHTFPA | GGACTGGTTCCTGGACCACTGGGGCCAGGGAACCC |
| | | VLQSSGLYSLSSVVTVPSS | TGGTCACCGTGTCCTCAGCCTCCACCAAGGGCCCAT |
| | | SLGTQTYICNVNHKPSNT | CGGTCTTCCCCCTGGCACCCTCCTCCAAGAGCACCT |
| | | KVDKKVEPKSCDKTHTC | CTGGGGGCACAGCGGCCCTGGGCTGCCTGGTCAAG |
| | | PPCPAPELLGGPSVFLFPP | GACTACTTCCCCGAACCGGTGACGGTGTCGTGGAA |
| | | KPKDTLMISRTPEVTCVV | CTCAGGCGCCCTGACCAGCGGCGTGCACACCTTCCC |
| | | VDVSHEDPEVKFNWYVD | GGCTGTCCTACAGTCCTCAGGACTCTACTCCCTCAG |
| | | GVEVHNAKTKPCEEQYG | CAGCGTGGTGACCGTGCCCTCCAGCAGCTTGGGCA |
| | | STYRCVSVLTVLHQDWL | CCCAGACCTACATCTGCAACGTGAATCACAAGCCC |
| | | NGKEYKCKVSNKALPAPI | AGCAACACCAAGGTGGACAAGAAAGTTGAGCCCAA |
| | | EKTISKAKGQPREPQVYT | ATCTTGTGACAAAACTCACACATGCCCACCGTGCCC |
| | | LPPSREEMTKNQVSLTCL | AGCACCTGAACTCCTGGGGGGACCGTCAGTCTTCCT |
| | | VKGFYPSDIAVEWESNGQ | CTTCCCCCCAAAACCCAAGGACACCCTCATGATCTC |
| | | PENNYKTTPPVLDSDGSF | CCGGACCCCTGAGGTCACATGCGTGGTGGTGGACG |
| | | FLYSKLTVDKSRWQQGN | TGAGCCACGAAGACCCTGAGGTCAAGTTCAACTGG |
| | | VFSCSVMHEALHNHYTQ | TACGTGGACGGCGTGGAGGTGCATAATGCCAAGAC |
| | | KSLSLSPGK (SEQ ID | AAAGCCGTGCGAGGAGCAGTACGGCAGCACGTACC |
| | | NO: 535) | GTTGCGTCAGCGTCCTCACCGTCCTGCACCAGGACT |
| | | | GGCTGAATGGCAAGGAGTACAAGTGCAAGGTGTCC |
| | | | AACAAAGCCCTCCCAGCCCCCATCGAGAAAACCAT |
| | | | CTCCAAAGCCAAAGGGCAGCCCCGAGAACCACAGG |
| | | | TGTACACCCTGCCCCCATCCCGGGAGGAGATGACC |
| | | | AAGAACCAGGTCAGCCTGACCTGCCTGGTCAAAGG |
| | | | CTTCTATCCCAGCGACATCGCCGTGGAGTGGGAGA |
| | | | GCAATGGGCAGCCGGAGAACAACTACAAGACCACG |
| | | | CCTCCCGTGCTGGACTCCGACGGCTCCTTCTTCCTCT |
| | | | ATAGCAAGCTCACCGTGGACAAGAGCAGGTGGCAG |
| | | | CAGGGGAACGTCTTCTCATGCTCCGTGATGCATGAG |
| | | | GCTCTGCACAACCACTACACGCAGAAGAGCCTCTC |
| | | | CCTGTCTCCGGGTAAA (SEQ ID NO: 568) |
| iPS:452128 | HC-14 | QVQLQQSGPGLVKPSQTL | CAGGTACAGCTGCAGCAGTCAGGTCCAGGACTGGT |
| | | SLTCAISGDSVSNKQATW | GAAGCCCTCGCAGACCCTCTCACTCACCTGTGCCAT |
| | | NWIRQSPSRGLEWLGRTY | CTCCGGGGACAGTGTCTCTAACAAACAGGCTACTTG |
| | | YRGKWKNHYAVSVKSRI | GAACTGGATCAGGCAGTCCCCATCGAGAGGCCTTG |
| | | TINPDTSKSQFSLQLNSVT | AGTGGCTGGGAAGGACATACTACAGGGGTAAATGG |
| | | PEDTAVYYCARGMWNQ | AAAAATCATTATGCAGTATCTGTGAAAAGTCGAAT |
| | | NWFLDHWGQGTLVTVSS | AACCATCAACCCCGACACGTCCAAGAGCCAGTTCT |
| | | ASTKGPSVFPLAPSSKSTS | CCCTGCAGCTGAACTCTGTGACTCCCGAGGACACG |
| | | GGTAALGCLVKDYFPEP | GCTGTGTATTACTGTGCAAGAGGAATGTGGAACCA |
| | | VTVSWNSGALTSGVHTFP | GAACTGGTTCCTGGACCACTGGGGCCAGGGAACCC |
| | | AVLQSSGLYSLSSVVTVP | TGGTCACCGTGTCCTCAGCCTCCACCAAGGGCCCAT |
| | | SSSLGTQTYICNVNHKPS | CGGTCTTCCCCCTGGCACCCTCCTCCAAGAGCACCT |
| | | NTKVDKKVEPKSCDKTH | CTGGGGGCACAGCGGCCCTGGGCTGCCTGGTCAAG |
| | | TCPPCPAPELLGGPSVFLF | GACTACTTCCCCGAACCGGTGACGGTGTCGTGGAA |
| | | PPKPKDTLMISRTPEVTC | CTCAGGCGCCCTGACCAGCGGCGTGCACACCTTCCC |
| | | VVVDVSHEDPEVKFNWY | GGCTGTCCTACAGTCCTCAGGACTCTACTCCCTCAG |
| | | VDGVEVHNAKTKPCEEQ | CAGCGTGGTGACCGTGCCCTCCAGCAGCTTGGGCA |
| | | YGSTYRCVSVLTVLHQD | CCCAGACCTACATCTGCAACGTGAATCACAAGCCC |
| | | WLNGKEYKCKVSNKALP | AGCAACACCAAGGTGGACAAGAAAGTTGAGCCCAA |
| | | APIEKTISKAKGQPREPQV | ATCTTGTGACAAAACTCACACATGCCCACCGTGCCC |
| | | YTLPPSREEMTKNQVSLT | AGCACCTGAACTCCTGGGGGGACCGTCAGTCTTCCT |
| | | CLVKGFYPSDIAVEWESN | CTTCCCCCCAAAACCCAAGGACACCCTCATGATCTC |
| | | GQPENNYKTTPPVLDSDG | CCGGACCCCTGAGGTCACATGCGTGGTGGTGGACG |
| | | SFFLYSKLTVDKSRWQQ | TGAGCCACGAAGACCCTGAGGTCAAGTTCAACTGG |
| | | GNVFSCSVMHEALHNHY | TACGTGGACGGCGTGGAGGTGCATAATGCCAAGAC |
| | | TQKSLSLSPGK (SEQ ID | AAAGCCGTGCGAGGAGCAGTACGGCAGCACGTACC |
| | | NO: 532) | GTTGCGTCAGCGTCCTCACCGTCCTGCACCAGGACT |
| | | | GGCTGAATGGCAAGGAGTACAAGTGCAAGGTGTCC |
| | | | AACAAAGCCCTCCCAGCCCCCATCGAGAAAACCAT |
| | | | CTCCAAAGCCAAAGGGCAGCCCCGAGAACCACAGG |
| | | | TGTACACCCTGCCCCCATCCCGGGAGGAGATGACC |
| | | | AAGAACCAGGTCAGCCTGACCTGCCTGGTCAAAGG |
| | | | CTTCTATCCCAGCGACATCGCCGTGGAGTGGGAGA |
| | | | GCAATGGGCAGCCGGAGAACAACTACAAGACCACG |
| | | | CCTCCCGTGCTGGACTCCGACGGCTCCTTCTTCCTCT |
| | | | ATAGCAAGCTCACCGTGGACAAGAGCAGGTGGCAG |
| | | | CAGGGGAACGTCTTCTCATGCTCCGTGATGCATGAG |
| | | | GCTCTGCACAACCACTACACGCAGAAGAGCCTCTC |
| | | | CCTGTCTCCGGGTAAA (SEQ ID NO: 565) |
| iPS:448924 | HC-18 | QVQLQQSGPGLVKPSQTL | CAGGTACAGCTGCAGCAGTCAGGTCCAGGACTGGT |
| | | SLTCAISGDSVSSRYATW | GAAGCCCTCGCAGACCCTCTCACTCACCTGTGCCAT |
| | | NWIRQSPSRGLEWLGRTY | CTCCGGGGACAGTGTCTCTTCTCGTTACGCTACTTG |
| | | YRGQWKNHYAVSVKSRI | GAACTGGATCAGGCAGTCCCCATCGAGAGGCCTTG |
| | | TINPDTSKSQFSLQLNSVT | AGTGGCTGGGAAGGACATACTACAGGGGTCAGTGG |
| | | PEDTAVYYCA | AAAAATCATTATGCAGTATCTGTGAAAAGTCGAAT |
| | | RGMWNQNWFLDHWGQ | AACCATCAACCCCGACACGTCCAAGAGCCAGTTCT |
| | | GTLVTVSSASTKGPSVFP | CCCTGCAGCTGAACTCTGTGACTCCCGAGGACACG |
| | | LAPSSKSTSGGTAALGCL | GCTGTGTATTACTGTGCAAGAGGAATGTGGAACCA |
| | | VKDYFPEPVTVSWNSGA | GAACTGGTTCCTGGACCACTGGGGCCAGGGAACCC |
| | | LTSGVHTFPAVLQSSGLY | TGGTCACCGTGTCCTCAGCCTCCACCAAGGGCCCAT |
| | | SLSSVVTVPSSSLGTQTYI | CGGTCTTCCCCCTGGCACCCTCCTCCAAGAGCACCT |
| | | CNVNHKPSNTKVDKKVE | CTGGGGGCACAGCGGCCCTGGGCTGCCTGGTCAAG |
| | | PKSCDKTHTCPPCPAPEL | GACTACTTCCCCGAACCGGTGACGGTGTCGTGGAA |
| | | LGGPSVFLFPPKPKDTLMI | CTCAGGCGCCCTGACCAGCGGCGTGCACACCTTCCC |
| | | SRTPEVTCVVVDVSHEDP | GGCTGTCCTACAGTCCTCAGGACTCTACTCCCTCAG |
| | | EVKFNWYVDGVEVHNA | CAGCGTGGTGACCGTGCCCTCCAGCAGCTTGGGCA |
| | | KTKPCEEQYGSTYRCVSV | CCCAGACCTACATCTGCAACGTGAATCACAAGCCC |
| | | LTVLHQDWLNGKEYKCK | AGCAACACCAAGGTGGACAAGAAAGTTGAGCCCAA |
| | | VSNKALPAPIEKTISKAKG | ATCTTGTGACAAAACTCACACATGCCCACCGTGCCC |
| | | QPREPQVYTLPPSREEMT | AGCACCTGAACTCCTGGGGGGACCGTCAGTCTTCCT |
| | | KNQVSLTCLVKGFYPSDI | CTTCCCCCCAAAACCCAAGGACACCCTCATGATCTC |
| | | AVEWESNGQPENNYKTT | CCGGACCCCTGAGGTCACATGCGTGGTGGTGGACG |
| | | PPVLDSDGSFFLYSKLTV | TGAGCCACGAAGACCCTGAGGTCAAGTTCAACTGG |
| | | DKSRWQQGNVFSCSVMH | TACGTGGACGGCGTGGAGGTGCATAATGCCAAGAC |
| | | EALHNHYTQKSLSLSPGK | AAAGCCGTGCGAGGAGCAGTACGGCAGCACGTACC |
| | | (SEQ ID NO: 536) | GTTGCGTCAGCGTCCTCACCGTCCTGCACCAGGACT |
| | | | GGCTGAATGGCAAGGAGTACAAGTGCAAGGTGTCC |
| | | | AACAAAGCCCTCCCAGCCCCCATCGAGAAAACCAT |
| | | | CTCCAAAGCCAAAGGGCAGCCCCGAGAACCACAGG |
| | | | TGTACACCCTGCCCCCATCCCGGGAGGAGATGACC |
| | | | AAGAACCAGGTCAGCCTGACCTGCCTGGTCAAAGG |
| | | | CTTCTATCCCAGCGACATCGCCGTGGAGTGGGAGA |
| | | | GCAATGGGCAGCCGGAGAACAACTACAAGACCACG |
| | | | CCTCCCGTGCTGGACTCCGACGGCTCCTTCTTCCTCT |
| | | | ATAGCAAGCTCACCGTGGACAAGAGCAGGTGGCAG |
| | | | CAGGGGAACGTCTTCTCATGCTCCGTGATGCATGAG |
| | | | GCTCTGCACAACCACTACACGCAGAAGAGCCTCTC |
| | | | CCTGTCTCCGGGTAAA (SEQ ID NO: 569) |
| 3574 | HC-14 | QVQLQQSGPGLVKPSQTL | CAGGTACAGCTGCAGCAGTCAGGTCCAGGACTGGT |
| | | SLTCAISGDSVSNKQATW | GAAGCCCTCGCAGACCCTCTCACTCACCTGTGCCAT |
| | | NWIRQSPSRGLEWLGRTY | CTCCGGGGACAGTGTCTCTAACAAACAGGCTACTTG |
| | | YRGKWKNHYAVSVKSRI | GAACTGGATCAGGCAGTCCCCATCGAGAGGCCTTG |
| | | TINPDTSKSQFSLQLNSVT | AGTGGCTGGGAAGGACATACTACAGGGGTAAATGG |
| | | PEDTAVYYCARGMWNQ | AAAAATCATTATGCAGTATCTGTGAAAAGTCGAAT |
| | | NWFLDHWGQGTLVTVSS | AACCATCAACCCCGACACGTCCAAGAGCCAGTTCT |
| | | ASTKGPSVFPLAPSSKSTS | CCCTGCAGCTGAACTCTGTGACTCCCGAGGACACG |
| | | GGTAALGCLVKDYFPEP | GCTGTGTATTACTGTGCAAGAGGAATGTGGAACCA |
| | | VTVSWNSGALTSGVHTFP | GAACTGGTTCCTGGACCACTGGGGCCAGGGAACCC |
| | | AVLQSSGLYSLSSVVTVP | TGGTCACCGTGTCCTCAGCCTCCACCAAGGGC |
| | | SSSLGTQTYICNVNHKPS | CCATCGGTCTTCCCCCTGGCACCCTCCTCCAAGAGC |
| | | NTKVDKKVEPKSCDKTH | ACCTCTGGGGGCACAGCGGCCCTGGGCTGCCTGGT |
| | | TCPPCPAPELLGGPSVFLF | CAAGGACTACTTCCCCGAACCGGTGACGGTGTCGT |
| | | PPKPKDTLMISRTPEVTC | GGAACTCAGGCGCCCTGACCAGCGGCGTGCACACC |
| | | VVVDVSHEDPEVKFNWY | TTCCCGGCTGTCCTACAGTCCTCAGGACTCTACTCC |
| | | VDGVEVHNAKTKPCEEQ | CTCAGCAGCGTGGTGACCGTGCCCTCCAGCAGCTTG |
| | | YGSTYRCVSVLTVLHQD | GGCACCCAGACCTACATCTGCAACGTGAATCACAA |
| | | WLNGKEYKCKVSNKALP | GCCCAGCAACACCAAGGTGGACAAGAAAGTTGAGC |
| | | APIEKTISKAKGQPREPQV | CCAAATCTTGTGACAAAACTCACACATGCCCACCGT |
| | | YTLPPSREEMTKNQVSLT | GCCCAGCACCTGAACTCCTGGGGGGACCGTCAGTC |
| | | CLVKGFYPSDIAVEWESN | TTCCTCTTCCCCCCAAAACCCAAGGACACCCTCATG |
| | | GQPENNYKTTPPVLDSDG | ATCTCCCGGACCCCTGAGGTCACATGCGTGGTGGTG |
| | | SFFLYSKLTVDKSRWQQ | GACGTGAGCCACGAAGACCCTGAGGTCAAGTTCAA |
| | | GNVFSCSVMHEALHNHY | CTGGTACGTGGACGGCGTGGAGGTGCATAATGCCA |
| | | TQKSLSLSPGK (SEQ ID | AGACAAAGCCGTGCGAGGAGCAGTACGGCAGCACG |
| | | NO: 532) | TACCGTTGCGTCAGCGTCCTCACCGTCCTGCACCAG |
| | | | GACTGGCTGAATGGCAAGGAGTACAAGTGCAAGGT |
| | | | GTCCAACAAAGCCCTCCCAGCCCCCATCGAGAAAA |
| | | | CCATCTCCAAAGCCAAAGGGCAGCCCCGAGAACCA |
| | | | CAGGTGTACACCCTGCCCCCATCCCGGGAGGAGAT |
| | | | GACCAAGAACCAGGTCAGCCTGACCTGCCTGGTCA |
| | | | AAGGCTTCTATCCCAGCGACATCGCCGTGGAGTGG |
| | | | GAGAGCAATGGGCAGCCGGAGAACAACTACAAGA |
| | | | CCACGCCTCCCGTGCTGGACTCCGACGGCTCCTTCT |
| | | | TCCTCTATAGCAAGCTCACCGTGGACAAGAGCAGG |
| | | | TGGCAGCAGGGGAACGTCTTCTCATGCTCCGTGATG |
| | | | CATGAGGCTCTGCACAACCACTACACGCAGAAGAG |
| | | | CCTCTCCCTGTCTCCGGGTAAA (SEQ ID NO: |
| | | | 565) |
| 3575 | HC-12 | QVQLQQSGPGLVKPSQTL | CAGGTACAGCTGCAGCAGTCAGGTCCAGGACTGGT |
| | | SLTCAISGDSVSSNSATW | GAAGCCCTCGCAGACCCTCTCACTCACCTGTGCCAT |
| | | NWIRQSPSRGLEWLGRTY | CTCCGGGGACAGTGTCTCTAGCAACAGTGCTACTTG |
| | | YRSKWSNHYAVSVKSRIT | GAACTGGATCAGGCAGTCCCCATCGAGAGGCCTTG |
| | | INPDTSKSQFSLQLNSVTP | AGTGGCTGGGAAGGACATATTACAGGTCCAAGTGG |
| | | EDTAVYYCARGTWKQL | TCTAATCATTATGCAGTATCTGTGAAAAGTCGAATC |
| | | WFLDHWGQGTLVTVSSA | ACCATCAACCCCGACACGTCCAAGAGCCAGTTCTCC |
| | | STKGPSVFPLAPSSKSTSG | CTGCAGCTGAACTCTGTGACTCCCGAGGACACGGCT |
| | | GTAALGCLVKDYFPEPVT | GTGTATTACTGTGCAAGAGGAACGTGGAAACAGCT |
| | | VSWNSGALTSGVHTFPA | ATGGTTCCTTGACCACTGGGGCCAGGGAACCCTGGT |
| | | VLQSSGLYSLSSVVTVPSS | CACCGTGTCTAGTGCCTCCACCAAGGGCCCATCGGT |
| | | SLGTQTYICNVNHKPSNT | CTTCCCCCTGGCACCCTCCTCCAAGAGCACCTCTGG |
| | | KVDKKVEPKSCDKTHTC | GGGCACAGCGGCCCTGGGCTGCCTGGTCAAGGACT |
| | | PPCPAPELLGGPSVFLFPP | ACTTCCCCGAACCGGTGACGGTGTCGTGGAACTCA |
| | | KPKDTLMISRTPEVTCVV | GGCGCCCTGACCAGCGGCGTGCACACCTTCCCGGCT |
| | | VDVSHEDPEVKFNWYVD | GTCCTACAGTCCTCAGGACTCTACTCCCTCAGCAGC |
| | | GVEVHNAKTKPCEEQYG | GTGGTGACCGTGCCCTCCAGCAGCTTGGGCACCCA |
| | | STYRCVSVLTVLHQDWL | GACCTACATCTGCAACGTGAATCACAAGCCCAGCA |
| | | NGKEYKCKVSNKALPAPI | ACACCAAGGTGGACAAGAAAGTTGAGCCCAAATCT |
| | | EKTISKAKGQPREPQVYT | TGTGACAAAACTCACACATGCCCACCGTGCCCAGC |
| | | LPPSREEMTKNQVSLTCL | ACCTGAACTCCTGGGGGGACCGTCAGTCTTCCTCTT |
| | | VKGFYPSDIAVEWESNGQ | CCCCCCAAAACCCAAGGACACCCTCATGATCTCCCG |
| | | PENNYKTTPPVLDSDGSF | GACCCCTGAGGTCACATGCGTGGTGGTGGACGTGA |
| | | FLYSKLTVDKSRWQQGN | GCCACGAAGACCCTGAGGTCAAGTTCAACTGGTAC |
| | | VFSCSVMHEALHNHYTQ | GTGGACGGCGTGGAGGTGCATAATGCCAAGACAAA |
| | | KSLSLSPGK (SEQ ID | GCCGTGCGAGGAGCAGTACGGCAGCACGTACCGTT |
| | | NO: 530) | GCGTCAGCGTCCTCACCGTCCTGCACCAGGACTGGC |
| | | | TGAATGGCAAGGAGTACAAGTGCAAGGTGTCCAAC |
| | | | AAAGCCCTCCCAGCCCCCATCGAGAAAACCATCTC |
| | | | CAAAGCCAAAGGGCAGCCCCGAGAACCACAGGTGT |
| | | | ACACCCTGCCCCCATCCCGGGAGGAGATGACCAAG |
| | | | AACCAGGTCAGCCTGACCTGCCTGGTCAAAGGCTTC |
| | | | TATCCCAGCGACATCGCCGTGGAGTGGGAGAGCAA |
| | | | TGGGCAGCCGGAGAACAACTACAAGACCACGCCTC |
| | | | CCGTGCTGGACTCCGACGGCTCCTTCTTCCTCTATA |
| | | | GCAAGCTCACCGTGGACAAGAGCAGGTGGCAGCAG |
| | | | GGGAACGTCTTCTCATGCTCCGTGATGCATGAGGCT |
| | | | CTGCACAACCACTACACGCAGAAGAGCCTCTCCCT |
| | | | GTCTCCGGGCAAA (SEQ ID NO: 563) |

In certain embodiments, the anti-PAC1 antibodies of the invention may comprise a light chain selected from LC-01 to LC-05, as shown in Table 4A, and/or a heavy chain selected from HC-01 to HC-11, as shown in Table 4B, and binding fragments, derivatives, and variants of these light chains and heavy chains. In other embodiments, the anti-PAC1 antibodies of the invention may comprise a light chain selected from LC-06 to LC-15, as shown in Table 4A, and/or a heavy chain selected from HC-12 to HC-18, as shown in Table 4B, and binding fragments, derivatives, and variants of these light chains and heavy chains.

Each of the light chains listed in Table 4A may be combined with any of the heavy chains listed in Table 4B to form an anti-PAC1 antibody or antigen-binding fragment thereof of the invention. For example, in certain embodiments, the anti-PAC1 antibodies of the invention comprise a light chain comprising the sequence of LC-03 (SEQ ID NO: 506) and a heavy chain comprising the sequence of HC-03 (SEQ ID NO: 521). In some embodiments, the anti-PAC1 antibodies of the invention comprise a light chain comprising the sequence of LC-03 (SEQ ID NO: 506) and a heavy chain comprising the sequence of HC-04 (SEQ ID NO: 522). In other embodiments, the anti-PAC1 antibodies of the invention comprise a light chain comprising the sequence of LC-01 (SEQ ID NO: 504) and a heavy chain comprising the sequence of HC-05 (SEQ ID NO: 523). In still other embodiments, the anti-PAC1 antibodies of the invention comprise a light chain comprising the sequence of LC-01 (SEQ ID NO: 504) and a heavy chain comprising the sequence of HC-06 (SEQ ID NO: 524). In some embodiments, the anti-PAC1 antibodies of the invention comprise a light chain comprising the sequence of LC-03 (SEQ ID NO: 506) and a heavy chain comprising the sequence of HC-07 (SEQ ID NO: 525). In certain embodiments, the anti-PAC1 antibodies of the invention comprise a light chain comprising the sequence of LC-01 (SEQ ID NO: 504) and a heavy chain comprising the sequence of HC-08 (SEQ ID NO: 526). In one embodiment, the anti-PAC1 antibodies of the invention comprise a light chain comprising the sequence of LC-03 (SEQ ID NO: 506) and a heavy chain comprising the sequence of HC-05 (SEQ ID NO: 523). In another embodiment, the anti-PAC1 antibodies of the invention comprise a light chain comprising the sequence of LC-04 (SEQ ID NO: 507) and a heavy chain comprising the sequence of HC-09 (SEQ ID NO: 527). In yet another embodiment, the anti-PAC1 antibodies of the invention comprise a light chain comprising the sequence of LC-04 (SEQ ID NO: 507) and a heavy chain comprising the sequence of HC-10 (SEQ ID NO: 528). In still another embodiment, the anti-PAC1 antibodies of the invention comprise a light chain comprising the sequence of LC-05 (SEQ ID NO: 508) and a heavy chain comprising the sequence of HC-11 (SEQ ID NO: 529).

In certain other embodiments, the anti-PAC1 antibodies of the invention comprise a light chain comprising the sequence of LC-07 (SEQ ID NO: 510) and a heavy chain comprising the sequence of HC-13 (SEQ ID NO: 531). In some embodiments, the anti-PAC1 antibodies of the invention comprise a light chain comprising the sequence of LC-08 (SEQ ID NO: 511) and a heavy chain comprising the sequence of HC-14 (SEQ ID NO: 532). In other embodiments, the anti-PAC1 antibodies of the invention comprise a light chain comprising the sequence of LC-09 (SEQ ID NO: 512) and a heavy chain comprising the sequence of HC-15 (SEQ ID NO: 533). In still other embodiments, the anti-PAC1 antibodies of the invention comprise a light chain comprising the sequence of LC-10 (SEQ ID NO: 513) and a heavy chain comprising the sequence of HC-16 (SEQ ID NO: 534). In some embodiments, the anti-PAC1 antibodies of the invention comprise a light chain comprising the sequence of LC-11 (SEQ ID NO: 514) and a heavy chain comprising the sequence of HC-17 (SEQ ID NO: 535). In certain embodiments, the anti-PAC1 antibodies of the invention comprise a light chain comprising the sequence of LC-12 (SEQ ID NO: 515) and a heavy chain comprising the sequence of HC-17 (SEQ ID NO: 535). In one embodiment, the anti-PAC1 antibodies of the invention comprise a light chain comprising the sequence of LC-13 (SEQ ID NO: 516) and a heavy chain comprising the sequence of HC-14 (SEQ ID NO: 532). In another embodiment, the anti-PAC1 antibodies of the invention comprise a light chain comprising the sequence of LC-14 (SEQ ID NO: 517) and a heavy chain comprising the sequence of HC-18 (SEQ ID NO: 536). In yet another embodiment, the anti-PAC1 antibodies of the invention comprise a light chain comprising the sequence of LC-06 (SEQ ID NO: 509) and a heavy chain comprising the sequence of HC-14 (SEQ ID NO: 532). In still another embodiment, the anti-PAC1 antibodies of the invention comprise a light chain comprising the sequence of LC-15 (SEQ ID NO: 518) and a heavy chain comprising the sequence of HC-12 (SEQ ID NO: 530).

In some embodiments, the anti-PAC1 antibodies comprise a light chain comprising a sequence of contiguous amino acids that differs from the sequence of a light chain in Table 4A, i.e. a light chain selected from LC-01 to LC-15, at only 1, 2, 3, 4, 5, 6, 7, 8, 9, 10, 11, 12, 13, 14 or 15 amino acid residues, wherein each such sequence difference is independently either a deletion, insertion or substitution of one amino acid, with the deletions, insertions and/or substitutions resulting in no more than 15 amino acid changes relative to the foregoing light chain sequences. The light chain in some anti-PAC1 antibodies comprises a sequence of amino acids that has at least 70%, at least 75%, at least 80%, at least 85%, at least 90%, at least 95%, at least 97% or at least 99% sequence identity to the amino acid sequences of SEQ ID NOs: 504 to 518 (i.e. the light chains in Table 4A).

In these and other embodiments, the anti-PAC1 antibodies comprise a heavy chain comprising a sequence of contiguous amino acids that differs from the sequence of a heavy chain in Table 4B, i.e., a heavy chain selected from HC-01 to HC-18, at only 1, 2, 3, 4, 5, 6, 7, 8, 9, 10, 11, 12, 13, 14 or 15 amino acid residues, wherein each such sequence difference is independently either a deletion, insertion or substitution of one amino acid, with the deletions, insertions and/or substitutions resulting in no more than 15 amino acid changes relative to the foregoing heavy chain sequences. The heavy chain in some anti-PAC1 antibodies comprises a sequence of amino acids that has at least 70%, at least 75%, at least 80%, at least 85%, at least 90%, at least 95%, at least 97% or at least 99% sequence identity to the amino acid sequences of SEQ ID NOs: 519 to 536 (i.e. the heavy chains in Table 4B).

The anti-PAC1 antibodies or antigen-binding fragments of the invention can be monoclonal antibodies, polyclonal antibodies, recombinant antibodies, human antibodies, humanized antibodies, chimeric antibodies, or multispecific antibodies or antigen-binding fragments thereof. In certain embodiments, the anti-PAC1 antibody is a monoclonal antibody. In such embodiments, the anti-PAC1 antibody may be a chimeric antibody, a humanized antibody, or a fully human antibody having a human immunoglobulin constant domain. In these and other embodiments, the anti-PAC1 antibody is a human IgG1, IgG2, IgG3, or IgG4 antibody. Thus, the anti-PAC1 antibody may, in some embodiments, have a human IgG1, IgG2, IgG3, or IgG4 constant domain. In one embodiment, the anti-PAC1 antibody is a monoclonal human IgG1 antibody. In another embodiment, the anti-PAC1 antibody is a monoclonal human IgG2 antibody. In yet another embodiment, the anti-PAC1 antibody is a monoclonal human IgG4 antibody.

The term “monoclonal antibody” (or “mAb”) as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against an individual antigenic site or epitope, in contrast to polyclonal antibody preparations that typically include different antibodies directed against different epitopes. Monoclonal antibodies may be produced using any technique known in the art, e.g., by immortalizing spleen cells harvested from an animal after completion of the immunization schedule. The spleen cells can be immortalized using any technique known in the art, e.g., by fusing them with myeloma cells to produce hybridomas. See, for example, Antibodies; Harlow and Lane, Cold Spring Harbor Laboratory Press, 1st Edition, e.g. from 1988, or 2nd Edition, e.g. from 2014. Myeloma cells for use in hybridoma-producing fusion procedures preferably are non-antibody-producing, have high fusion efficiency, and enzyme deficiencies that render them incapable of growing in certain selective media, which support the growth of only the desired fused cells (hybridomas). Examples of suitable cell lines for use in fusions with mouse cells include, but are not limited to, Sp-20, P3-X63/Ag8, P3-X63-Ag8.653, NS1/1.Ag 4 1, Sp210-Ag14, FO, NSO/U, MPC-11, MPC11-X45-GTG 1.7 and 5194/5XXO Bul. Examples of suitable cell lines used for fusions with rat cells include, but are not limited to, R210.RCY3, Y3-Ag 1.2.3, IR983F and 4B210. Other cell lines useful for cell fusions are U-266, GM1500-GRG2, LICR-LON-HMy2 and UC729-6.

In some instances, a hybridoma cell line is produced by immunizing an animal (e.g., a rabbit, rat, mouse, or a transgenic animal having human immunoglobulin sequences) with a PAC1 immunogen (see, e.g., WO 2014/144632); harvesting spleen cells from the immunized animal; fusing the harvested spleen cells to a myeloma cell line, thereby generating hybridoma cells; establishing hybridoma cell lines from the hybridoma cells, and identifying a hybridoma cell line that produces an antibody that binds to PAC1. Another useful method for producing monoclonal antibodies is the SLAM method described in Babcook et al., Proc. Natl. Acad. Sci. USA, Vol. 93: 7843-7848, 1996.

Monoclonal antibodies secreted by a hybridoma cell line can be purified using any technique known in the art, such as protein A-Sepharose, hydroxyapatite chromatography, gel electrophoresis, dialysis, or affinity chromatography. Hybridoma supernatants or mAbs may be further screened to identify mAbs with particular properties, such as the ability to bind PAC1 (e.g. human PAC1, cynomolgus monkey PAC1, or rat PAC1); cross-reactivity to other PAC1 family members (e.g. human VPAC1 or human VPAC2); ability to block or interfere with the binding of the PACAP ligand to PAC1, or the ability to functionally block PACAP-induced activation of the PAC1 receptor, e.g., using a cAMP assay as described herein.

In some embodiments, the anti-PAC1 antibodies or antigen-binding fragments of the invention are chimeric or humanized antibodies or antigen-binding fragments thereof based upon the CDR and variable region sequences of the antibodies described herein. A chimeric antibody is an antibody composed of protein segments from different antibodies that are covalently joined to produce functional immunoglobulin light or heavy chains or binding fragments thereof. Generally, a portion of the heavy chain and/or light chain is identical with or homologous to a corresponding sequence in antibodies derived from a particular species or belonging to a particular antibody class or subclass, while the remainder of the chain(s) is/are identical with or homologous to a corresponding sequence in antibodies derived from another species or belonging to another antibody class or subclass. For methods relating to chimeric antibodies, see, for example, U.S. Pat. No. 4,816,567 and Morrison et al., 1985, Proc. Natl. Acad. Sci. USA 81:6851-6855, both of which are hereby incorporated by reference in their entireties.

Generally, the goal of making a chimeric antibody is to create a chimera in which the number of amino acids from the intended species is maximized. One example is the “CDR-grafted” antibody, in which the antibody comprises one or more CDRs from a particular species or belonging to a particular antibody class or subclass, while the remainder of the antibody chain(s) is/are identical with or homologous to a corresponding sequence in antibodies derived from another species or belonging to another antibody class or subclass. CDR grafting is described, for example, in U.S. Pat. Nos. 6,180,370, 5,693,762, 5,693,761, 5,585,089, and 5,530,101. For use in humans, the variable region or selected CDRs from a rodent or rabbit antibody often are grafted into a human antibody, replacing the naturally-occurring variable regions or CDRs of the human antibody.

One useful type of chimeric antibody is a “humanized” antibody. Generally, a humanized antibody is produced from a monoclonal antibody raised initially in a non-human animal, such as a rodent or rabbit. Certain amino acid residues in this monoclonal antibody, typically from non-antigen recognizing portions of the antibody, are modified to be homologous to corresponding residues in a human antibody of corresponding isotype. Humanization can be performed, for example, using various methods by substituting at least a portion of a rodent or rabbit variable region for the corresponding regions of a human antibody (see, e.g., U.S. Pat. Nos. 5,585,089, and 5,693,762; Jones et al., 1986, Nature 321:522-525; Riechmann et al., 1988, Nature 332:323-27; and Verhoeyen et al., 1988, Science 239:1534-1536).

In one aspect, the CDRs of the light and heavy chain variable regions of the antibodies provided herein (see, Tables 1A and 1B) are grafted to framework regions (FRs) from antibodies from the same, or a different, phylogenetic species. For example, the CDRs of the heavy and light chain variable regions listed in Tables 1A and 1B can be grafted to consensus human FRs. To create consensus human FRs, FRs from several human heavy chain or light chain amino acid sequences may be aligned to identify a consensus amino acid sequence. Alternatively, the grafted variable regions from the one heavy or light chain may be used with a constant region that is different from the constant region of that particular heavy or light chain as disclosed herein. In other embodiments, the grafted variable regions are part of a single chain Fv antibody.

In certain embodiments, the anti-PAC1 antibodies or antigen-binding fragments of the invention are fully human antibodies or antigen-binding fragments thereof. Fully human antibodies that specifically bind to human PAC1 can be generated using the immunogens or fragments thereof described in WO 2014/144632, such as polypeptides consisting of the sequence of SEQ ID NO: 1 or SEQ ID NO: 4. A “fully human antibody” is an antibody that comprises variable and constant regions derived from or indicative of human germ line immunoglobulin sequences. One specific means provided for implementing the production of fully human antibodies is the “humanization” of the mouse humoral immune system. Introduction of human immunoglobulin (Ig) loci into mice in which the endogenous Ig genes have been inactivated is one means of producing fully human monoclonal antibodies (mAbs) in mouse, an animal that can be immunized with any desirable antigen. Using fully human antibodies can minimize the immunogenic and allergic responses that can sometimes be caused by administering mouse or mouse-derived mAbs to humans as therapeutic agents.

Fully human antibodies can be produced by immunizing transgenic animals (usually mice) that are capable of producing a repertoire of human antibodies in the absence of endogenous immunoglobulin production. Antigens for this purpose typically have six or more contiguous amino acids, and optionally are conjugated to a carrier, such as a hapten. See, e.g., Jakobovits et al., 1993, Proc. Natl. Acad. Sci. USA 90:2551-2555; Jakobovits et al., 1993, Nature 362:255-258; and Bruggermann et al., 1993, Year in Immunol. 7:33. In one example of such a method, transgenic animals are produced by incapacitating the endogenous mouse immunoglobulin loci encoding the mouse heavy and light immunoglobulin chains therein, and inserting into the mouse genome large fragments of human genome DNA containing loci that encode human heavy and light chain proteins. Partially modified animals, which have less than the full complement of human immunoglobulin loci, are then cross-bred to obtain an animal having all of the desired immune system modifications. When administered an immunogen, these transgenic animals produce antibodies that are immunospecific for the immunogen but have human rather than murine amino acid sequences, including the variable regions. For further details of such methods, see, for example, WO96/33735 and WO94/02602. Additional methods relating to transgenic mice for making human antibodies are described in U.S. Pat. Nos. 5,545,807; 6,713,610; 6,673,986; 6,162,963; 5,939,598; 5,545,807; 6,300,129; 6,255,458; 5,877,397; 5,874,299 and 5,545,806; in PCT publications WO91/10741, WO90/04036, WO 94/02602, WO 96/30498, WO 98/24893 and in EP 546073B1 and EP 546073A1.

The transgenic mice described above, referred to as “HuMab” mice, contain a human immunoglobulin gene minilocus that encodes unrearranged human heavy (mu and gamma) and kappa light chain immunoglobulin sequences, together with targeted mutations that inactivate the endogenous mu and kappa chain loci (Lonberg et al., 1994, Nature 368:856-859). Accordingly, the mice exhibit reduced expression of mouse IgM and kappa proteins and in response to immunization, the introduced human heavy and light chain transgenes undergo class switching and somatic mutation to generate high affinity human IgG kappa monoclonal antibodies (Lonberg and Huszar, 1995, Intern. Rev. Immunol. 13: 65-93; Harding and Lonberg, 1995, Ann. N.Y Acad. Sci. 764:536-546). The preparation of HuMab mice is described in detail in Taylor et al., 1992, Nucleic Acids Research 20:6287-6295; Chen et al., 1993, International Immunology 5:647-656; Tuaillon et al., 1994, J. Immunol. 152:2912-2920; Lonberg et al., 1994, Nature 368:856-859; Lonberg, 1994, Handbook of Exp. Pharmacology 113:49-101; Taylor et al., 1994, International Immunology 6:579-591; Lonberg and Huszar, 1995, Intern. Rev. Immunol. 13:65-93; Harding and Lonberg, 1995, Ann. N.Y Acad. Sci. 764:536-546; Fishwild et al., 1996, Nature Biotechnology 14:845-851; the foregoing references are hereby incorporated by reference in their entireties. See, further U.S. Pat. Nos. 5,545,806; 5,569,825; 5,625,126; 5,633,425; 5,789,650; 5,877,397; 5,661,016; 5,814,318; 5,874,299; and 5,770,429; as well as U.S. Pat. No. 5,545,807; International Publication Nos. WO 93/1227; WO 92/22646; and WO 92/03918, the disclosures of all of which are hereby incorporated by reference in their entireties. Technologies utilized for producing human antibodies in these transgenic mice are disclosed also in WO 98/24893, and Mendez et al., 1997, Nature Genetics 15:146-156, which are hereby incorporated by reference. For example, the HCo7 and HCo12 transgenic mice strains can be used to generate fully human anti-PAC1 antibodies. One particular transgenic mouse line suitable for generation of fully human anti-PAC1 antibodies is the XenoMouse® transgenic mouse line described in U.S. Pat. Nos. 6,114,598; 6,162,963; 6,833,268; 7,049,426; 7,064,244; Green et al., 1994, Nature Genetics 7:13-21; Mendez et al., 1997, Nature Genetics 15:146-156; Green and Jakobovitis, 1998, J. Ex. Med, 188:483-495; Green, 1999, Journal of Immunological Methods 231:11-23; Kellerman and Green, 2002, Current Opinion in Biotechnology 13, 593-597, all of which are hereby incorporated by reference in their entireties.

Human-derived antibodies can also be generated using phage display techniques. Phage display is described in e.g., Dower et al., WO 91/17271, McCafferty et al., WO 92/01047, and Caton and Koprowski, 1990, Proc. Natl. Acad. Sci. USA, 87:6450-6454, each of which is incorporated herein by reference in its entirety. The antibodies produced by phage technology are usually produced as antigen-binding fragments, e.g. Fv or Fab fragments, in bacteria and thus lack effector functions. Effector functions can be introduced by one of two strategies: The fragments can be engineered either into complete antibodies for expression in mammalian cells, or into bispecific antibody fragments with a second binding site capable of triggering an effector function, if desired. Typically, the Fd fragment (VH-CH1) and light chain (VL-CL) of antibodies are separately cloned by PCR and recombined randomly in combinatorial phage display libraries, which can then be selected for binding to a particular antigen. The antibody fragments are expressed on the phage surface, and selection of Fv or Fab (and therefore the phage containing the DNA encoding the antibody fragment) by antigen binding is accomplished through several rounds of antigen binding and re-amplification, a procedure termed panning. Antibody fragments specific for the antigen are enriched and finally isolated. Phage display techniques can also be used in an approach for the humanization of rodent monoclonal antibodies, called “guided selection” (see Jespers, L. S. et al., 1994, Bio/Technology 12, 899-903). For this, the Fd fragment of the mouse monoclonal antibody can be displayed in combination with a human light chain library, and the resulting hybrid Fab library may then be selected with antigen. The mouse Fd fragment thereby provides a template to guide the selection. Subsequently, the selected human light chains are combined with a human Fd fragment library. Selection of the resulting library yields entirely human Fab.

Once cells producing anti-PAC1 antibodies according to the invention have been obtained using any of the above described immunization and other techniques, the specific antibody genes may be cloned by isolating and amplifying DNA or mRNA therefrom according to standard procedures as described herein. The antibodies produced therefrom may be sequenced and the CDRs identified and the DNA coding for the CDRs may be manipulated as described herein to generate other anti-PAC1 antibodies or antigen-binding fragments according to the invention.

In certain embodiments, the anti-PAC1 antibodies and antigen-binding fragments of the invention may comprise one or more mutations or modifications to a constant region. For example, the heavy chain constant regions or the Fc regions of the anti-PAC1 antibodies may comprise one or more amino acid substitutions that affect the glycosylation, effector function, and/or Fey receptor binding of the antibody. The term “Fc region” refers to the C-terminal region of an immunoglobulin heavy chain, which may be generated by papain digestion of an intact antibody. The Fc region of an immunoglobulin generally comprises two constant domains, a CH2 domain and a CH3 domain, and optionally comprises a CH4 domain. In certain embodiments, the Fc region is an Fc region from an IgG1, IgG2, IgG3, or IgG4 immunoglobulin. In some embodiments, the Fc region comprises CH2 and CH3 domains from a human IgG1 or human IgG2 immunoglobulin. The Fc region may retain effector function, such as C1q binding, complement-dependent cytotoxicity (CDC), Fc receptor binding, antibody-dependent cell-mediated cytotoxicity (ADCC), and phagocytosis. In other embodiments, the Fc region may be modified to reduce or eliminate effector function as described in further detail below.

Unless indicated otherwise by reference to a specific sequence, throughout the present specification and claims, the numbering of the amino acid residues in an immunoglobulin heavy chain or light chain is according to AHo numbering as described in Honegger and Pluckthun, J. Mol. Biol. 309(3):657-670; 2001 or EU numbering as described in Edelman et al., Proc. Natl. Acad. USA, Vol. 63: 78-85 (1969). As used herein, the AHo numbering scheme is typically used when referring to the position of an amino acid within the variable regions, whereas the EU numbering scheme is generally used when referring to the position of an amino acid with an immunoglobulin constant region.

An amino acid substitution in an amino acid sequence is typically designated herein with a one-letter abbreviation for the amino acid residue in a particular position, followed by the numerical amino acid position relative to an original sequence of interest, which is then followed by the one-letter abbreviation for the amino acid residue substituted in. For example, “T30D” symbolizes a substitution of a threonine residue by an aspartate residue at amino acid position 30, relative to the original sequence of interest. Another example, “S218G” symbolizes a substitution of a serine residue by a glycine residue at amino acid position 218, relative to the original amino acid sequence of interest.

One of the functions of the Fc region of an immunoglobulin is to communicate to the immune system when the immunoglobulin binds its target. This is commonly referred to as “effector function.” Communication leads to antibody-dependent cellular cytotoxicity (ADCC), antibody-dependent cellular phagocytosis (ADCP), and/or complement dependent cytotoxicity (CDC). ADCC and ADCP are mediated through the binding of the Fc region to Fc receptors on the surface of cells of the immune system. CDC is mediated through the binding of the Fc region with proteins of the complement system, e.g., C1q. In some embodiments, the anti-PAC1 antibodies of the invention comprise one or more amino acid substitutions in the Fc region to enhance effector function, including ADCC activity, CDC activity, ADCP activity, and/or the clearance or half-life of the antibody. Exemplary amino acid substitutions (according to the EU numbering scheme) that can enhance effector function include, but are not limited to, E233L, L234I, L234Y, L235S, G236A, S239D, F243L, F243V, P247I, D280H, K290S, K290E, K290N, K290Y, R292P, E294L, Y296W, S298A, S298D, S298V, S298G, S298T, T299A, Y300L, V305I, Q311M, K326A, K326E, K326W, A330S, A330L, A330M, A330F, I332E, D333A, E333S, E333A, K334A, K334V, A339D, A339Q, P396L, or combinations of any of the foregoing.

In other embodiments, the anti-PAC1 antibodies of the invention comprise one or more amino acid substitutions in the Fc region to reduce effector function. Exemplary amino acid substitutions (according to the EU numbering scheme) that can reduce effector function include, but are not limited to, C220S, C226S, C229S, E233P, L234A, L234V, V234A, L234F, L235A, L235E, G237A, P238S, S267E, H268Q, N297A, N297G, V309L, E318A, L328F, A330S, A331S, P331S, or combinations of any of the foregoing.

Glycosylation can contribute to the effector function of antibodies, particularly IgG1 antibodies. Thus, in some embodiments, the anti-PAC1 antibodies of the invention may comprise one or more amino acid substitutions that affect the level or type of glycosylation of the antibodies. Glycosylation of polypeptides is typically either N-linked or O-linked. N-linked refers to the attachment of the carbohydrate moiety to the side chain of an asparagine residue. The tri-peptide sequences asparagine-X-serine and asparagine-X-threonine, where X is any amino acid except proline, are the recognition sequences for enzymatic attachment of the carbohydrate moiety to the asparagine side chain. Thus, the presence of either of these tri-peptide sequences in a polypeptide creates a potential glycosylation site. O-linked glycosylation refers to the attachment of one of the sugars N-acetylgalactosamine, galactose, or xylose, to a hydroxyamino acid, most commonly serine or threonine, although 5-hydroxyproline or 5-hydroxylysine may also be used.

In certain embodiments, glycosylation of the anti-PAC1 antibodies described herein is increased by adding one or more glycosylation sites, e.g., to the Fc region of the antibody. Addition of glycosylation sites to the antibody can be conveniently accomplished by altering the amino acid sequence such that it contains one or more of the above-described tri-peptide sequences (for N-linked glycosylation sites). The alteration may also be made by the addition of, or substitution by, one or more serine or threonine residues to the starting sequence (for O-linked glycosylation sites). For ease, the antibody amino acid sequence may be altered through changes at the DNA level, particularly by mutating the DNA encoding the target polypeptide at preselected bases such that codons are generated that will translate into the desired amino acids.

The invention also encompasses production of antibody molecules with altered carbohydrate structure resulting in altered effector activity, including antibodies with absent or reduced fucosylation that exhibit improved ADCC activity. Various methods are known in the art to reduce or eliminate fucosylation. For example, ADCC effector activity is mediated by binding of the antibody molecule to the FcγRIII receptor, which has been shown to be dependent on the carbohydrate structure of the N-linked glycosylation at the N297 residue of the CH2 domain. Non-fucosylated antibodies bind this receptor with increased affinity and trigger FcγRIII-mediated effector functions more efficiently than native, fucosylated antibodies. For example, recombinant production of non-fucosylated antibody in CHO cells in which the alpha-1,6-fucosyl transferase enzyme has been knocked out results in antibody with 100-fold increased ADCC activity (see Yamane-Ohnuki et al., Biotechnol Bioeng. 87(5):614-22, 2004). Similar effects can be accomplished through decreasing the activity of alpha-1,6-fucosyl transferase enzyme or other enzymes in the fucosylation pathway, e.g., through siRNA or antisense RNA treatment, engineering cell lines to knockout the enzyme(s), or culturing with selective glycosylation inhibitors (see Rothman et al., Mol Immunol. 26(12):1113-23, 1989). Some host cell strains, e.g. Lec13 or rat hybridoma YB2/0 cell line naturally produce antibodies with lower fucosylation levels (see Shields et al., J Biol Chem. 277(30):26733-40, 2002 and Shinkawa et al., J Biol Chem. 278(5):3466-73, 2003). An increase in the level of bisected carbohydrate, e.g. through recombinantly producing antibody in cells that overexpress GnTIII enzyme, has also been determined to increase ADCC activity (see Umana et al., Nat Biotechnol. 17(2):176-80, 1999).

In other embodiments, glycosylation of the anti-PAC1 antibodies described herein is decreased or eliminated by removing one or more glycosylation sites, e.g., from the Fc region of the antibody. In some embodiments, the anti-PAC1 antibody is an aglycosylated human monoclonal antibody, e.g., an aglycosylated human IgG1 monoclonal antibody. Amino acid substitutions that eliminate or alter N-linked glycosylation sites can reduce or eliminate N-linked glycosylation of the antibody. In certain embodiments, the anti-PAC1 antibodies described herein comprise a heavy chain mutation at position N297 (according to the EU numbering scheme), such as N297Q, N297A, or N297G. In some embodiments, the anti-PAC1 antibodies of the invention comprise an Fc region from a human IgG1 antibody with a mutation at position N297. In one particular embodiment, the anti-PAC1 antibodies of the invention comprise an Fc region from a human IgG1 antibody with a N297G mutation. For instance, in some embodiments, the anti-PAC1 antibodies of the invention comprise a heavy chain constant region comprising the sequence of SEQ ID NO: 324.

To improve the stability of molecules comprising a N297 mutation, the Fc region of the anti-PAC1 antibodies may be further engineered. For instance, in some embodiments, one or more amino acids in the Fc region are substituted with cysteine to promote disulfide bond formation in the dimeric state. Residues corresponding to V259, A287, R292, V302, L306, V323, or I332 (according to the EU numbering scheme) of an IgG1 Fc region may thus be substituted with cysteine. Preferably, specific pairs of residues are substituted with cysteine such that they preferentially form a disulfide bond with each other, thus limiting or preventing disulfide bond scrambling. Preferred pairs include, but are not limited to, A287C and L306C, V259C and L306C, R292C and V302C, and V323C and I332C. In certain embodiments, the anti-PAC1 antibodies described herein comprise an Fc region from a human IgG1 antibody with mutations R292C and V302C. In such embodiments, the Fc region may also comprise a N297 mutation, such as a N297G mutation. In some embodiments, the anti-PAC1 antibodies of the invention comprise a heavy chain constant region comprising the sequence of SEQ ID NO: 325.

Modifications of the anti-PAC1 antibodies of the invention to increase serum half-life also may desirable, for example, by incorporation of or addition of a salvage receptor binding epitope (e.g., by mutation of the appropriate region or by incorporating the epitope into a peptide tag that is then fused to the antibody at either end or in the middle, e.g., by DNA or peptide synthesis; see, e.g., WO96/32478) or adding molecules such as PEG or other water soluble polymers, including polysaccharide polymers. The salvage receptor binding epitope preferably constitutes a region wherein any one or more amino acid residues from one or two loops of an Fc region are transferred to an analogous position in the antibody. Even more preferably, three or more residues from one or two loops of the Fc region are transferred. Still more preferred, the epitope is taken from the CH2 domain of the Fc region (e.g., an IgG Fc region) and transferred to the CH1, CH3, or VH region, or more than one such region, of the antibody. Alternatively, the epitope is taken from the CH2 domain of the Fc region and transferred to the CL region or VL region, or both, of the antibody. See International applications WO 97/34631 and WO 96/32478 for a description of Fc variants and their interaction with the salvage receptor.

The present invention includes one or more isolated polynucleotides or isolated nucleic acids encoding the anti-PAC1 antibodies or antigen-binding fragments described herein. In addition, the present invention encompasses vectors comprising the nucleic acids, host cells or cell lines comprising the nucleic acids, and methods of making the anti-PAC1 antibodies and antigen-binding fragments of the invention. The nucleic acids comprise, for example, polynucleotides that encode all or part of an antibody or antigen-binding fragment, for example, one or both chains of an antibody of the invention, or a fragment, derivative, or variant thereof, polynucleotides sufficient for use as hybridization probes, PCR primers or sequencing primers for identifying, analyzing, mutating or amplifying a polynucleotide encoding a polypeptide, anti-sense oligonucleotides for inhibiting expression of a polynucleotide, and complementary sequences of the foregoing. The nucleic acids can be any length as appropriate for the desired use or function, and can comprise one or more additional sequences, for example, regulatory sequences, and/or be part of a larger nucleic acid, for example, a vector. Nucleic acid molecules of the invention include DNA and RNA in both single-stranded and double-stranded form, as well as the corresponding complementary sequences. DNA includes, for example, cDNA, genomic DNA, chemically synthesized DNA, DNA amplified by PCR, and combinations thereof. The nucleic acid molecules of the invention include full-length genes or cDNA molecules as well as a combination of fragments thereof. The nucleic acids of the invention can be derived from human sources as well as non-human species.

Relevant amino acid sequences from an immunoglobulin or region thereof (e.g. variable region, Fc region, etc.) or polypeptide of interest may be determined by direct protein sequencing, and suitable encoding nucleotide sequences can be designed according to a universal codon table. Alternatively, genomic or cDNA encoding monoclonal antibodies or binding fragments thereof of the invention can be isolated and sequenced from cells producing such antibodies using conventional procedures (e.g., by using oligonucleotide probes that are capable of binding specifically to genes encoding the heavy and light chains of the monoclonal antibodies).

An “isolated nucleic acid,” which is used interchangeably herein with “isolated polynucleotide,” is a nucleic acid that has been separated from adjacent genetic sequences present in the genome of the organism from which the nucleic acid was isolated, in the case of nucleic acids isolated from naturally-occurring sources. In the case of nucleic acids synthesized enzymatically from a template or chemically, such as PCR products, cDNA molecules, or oligonucleotides for example, it is understood that the nucleic acids resulting from such processes are isolated nucleic acids. An isolated nucleic acid molecule refers to a nucleic acid molecule in the form of a separate fragment or as a component of a larger nucleic acid construct. In one preferred embodiment, the nucleic acids are substantially free from contaminating endogenous material. The nucleic acid molecule has preferably been derived from DNA or RNA isolated at least once in substantially pure form and in a quantity or concentration enabling identification, manipulation, and recovery of its component nucleotide sequences by standard biochemical methods (such as those outlined in Sambrook et al., Molecular Cloning: A Laboratory Manual, 2nd ed., Cold Spring Harbor Laboratory, Cold Spring Harbor, NY (1989)). Such sequences are preferably provided and/or constructed in the form of an open reading frame uninterrupted by internal non-translated sequences, or introns, that are typically present in eukaryotic genes. Sequences of non-translated DNA can be present 5′ or 3′ from an open reading frame, where the same do not interfere with manipulation or expression of the coding region. Unless specified otherwise, the left-hand end of any single-stranded polynucleotide sequence discussed herein is the 5′ end; the left-hand direction of double-stranded polynucleotide sequences is referred to as the 5′ direction. The direction of 5′ to 3′ production of nascent RNA transcripts is referred to as the transcription direction; sequence regions on the DNA strand having the same sequence as the RNA transcript that are 5′ to the 5′ end of the RNA transcript are referred to as “upstream sequences;” sequence regions on the DNA strand having the same sequence as the RNA transcript that are 3′ to the 3′ end of the RNA transcript are referred to as “downstream sequences.”

The present invention also includes nucleic acids that hybridize under moderately stringent conditions, and more preferably highly stringent conditions, to nucleic acids encoding polypeptides as described herein. The basic parameters affecting the choice of hybridization conditions and guidance for devising suitable conditions are set forth by Sambrook, Fritsch, and Maniatis (1989, Molecular Cloning: A Laboratory Manual, Cold Spring Harbor Laboratory Press, Cold Spring Harbor, N.Y., chapters 9 and 11; and Current Protocols in Molecular Biology, 1995, Ausubel et al., eds., John Wiley & Sons, Inc., sections 2.10 and 6.3-6.4), and can be readily determined by those having ordinary skill in the art based on, for example, the length and/or base composition of the DNA. One way of achieving moderately stringent conditions involves the use of a prewashing solution containing 5×SSC, 0.5% SDS, 1.0 mM EDTA (pH 8.0), hybridization buffer of about 50% formamide, 6×SSC, and a hybridization temperature of about 55° C. (or other similar hybridization solutions, such as one containing about 50% formamide, with a hybridization temperature of about 42° C.), and washing conditions of about 60° C., in 0.5×SSC, 0.1% SDS. Generally, highly stringent conditions are defined as hybridization conditions as above, but with washing at approximately 68° C., 0.2×SSC, 0.1% SDS. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes after hybridization is complete. It should be understood that the wash temperature and wash salt concentration can be adjusted as necessary to achieve a desired degree of stringency by applying the basic principles that govern hybridization reactions and duplex stability, as known to those skilled in the art and described further below (see, e.g., Sambrook et al., 1989).

When hybridizing a nucleic acid to a target nucleic acid of unknown sequence, the hybrid length is assumed to be that of the hybridizing nucleic acid. When nucleic acids of known sequence are hybridized, the hybrid length can be determined by aligning the sequences of the nucleic acids and identifying the region or regions of optimal sequence complementarity. The hybridization temperature for hybrids anticipated to be less than 50 base pairs in length should be 5 to 10° C. less than the melting temperature (Tm) of the hybrid, where Tm is determined according to the following equations. For hybrids less than 18 base pairs in length, Tm (° C.)=2(#of A+T bases)+4(#of G+C bases). For hybrids above 18 base pairs in length, Tm (° C.)=81.5+16.6(log 10 [Na+])+0.41(% G+C)−(600/N), where N is the number of bases in the hybrid, and [Na+] is the concentration of sodium ions in the hybridization buffer ([Na+] for 1×SSC=0.165M). Preferably, each such hybridizing nucleic acid has a length that is at least 15 nucleotides (or more preferably at least 18 nucleotides, or at least 20 nucleotides, or at least 25 nucleotides, or at least 30 nucleotides, or at least 40 nucleotides, or most preferably at least 50 nucleotides), or at least 25% (more preferably at least 50%, or at least 60%, or at least 70%, and most preferably at least 80%) of the length of the nucleic acid of the present invention to which it hybridizes, and has at least 60% sequence identity (more preferably at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, and most preferably at least 99.5%) with the nucleic acid of the present invention to which it hybridizes, where sequence identity is determined by comparing the sequences of the hybridizing nucleic acids when aligned so as to maximize overlap and identity while minimizing sequence gaps as described in more detail above.

Variants of the anti-PAC1 antibodies and antigen-binding fragments described herein can be prepared by site-specific mutagenesis of nucleotides in the DNA encoding the polypeptide, using cassette or PCR mutagenesis or other techniques well known in the art, such as those described in Example 3, to produce DNA encoding the variant, and thereafter expressing the recombinant DNA in cell culture as outlined herein. However, antibodies or antigen-binding fragments comprising variant CDRs having up to about 100-150 residues may be prepared by in vitro synthesis using established techniques. The variants typically exhibit the same qualitative biological activity as the naturally occurring analogue, e.g., binding to antigen. Such variants include, for example, deletions and/or insertions and/or substitutions of residues within the amino acid sequences of the antibodies. Any combination of deletion, insertion, and substitution is made to arrive at the final construct, provided that the final construct possesses the desired characteristics. The amino acid changes also may alter post-translational processes of the antibody, such as changing the number or position of glycosylation sites. In certain embodiments, antibody variants are prepared with the intent to modify those amino acid residues which are directly involved in epitope binding. In other embodiments, modification of residues which are not directly involved in epitope binding or residues not involved in epitope binding in any way, is desirable, for purposes discussed herein. Mutagenesis within any of the CDR regions and/or framework regions is contemplated. Covariance analysis techniques can be employed by the skilled artisan to design useful modifications in the amino acid sequence of the antibody. See, e.g., Choulier, et al., Proteins 41:475-484, 2000; Demarest et al., J. Mol. Biol. 335:41-48, 2004; Hugo et al., Protein Engineering 16(5):381-86, 2003; Aurora et al., US Patent Publication No. 2008/0318207 A1; Glaser et al., US Patent Publication No. 2009/0048122 A1; Urech et al., WO 2008/110348 A1; Borras et al., WO 2009/000099 A2. Such modifications determined by covariance analysis can improve potency, pharmacokinetic, pharmacodynamic, and/or manufacturability characteristics of an antibody.

Tables 4A and 4B show exemplary nucleic acid sequences encoding the full light and heavy chains, respectively, of anti-PAC1 antibodies described herein. Tables 5A and 5B show exemplary nucleic acid sequences encoding the light and heavy chain variable regions, respectively, of anti-PAC1 antibodies described herein. Polynucleotides encoding the anti-PAC1 variable regions can be used, optionally with nucleic acids encoding the light and heavy chain constant regions listed in Tables 2 and 3, respectively, to construct the antibodies and antigen-binding fragments of the invention.

TABLE 5A
Exemplary Anti-PAC1 Antibody Light Chain Variable Region Nucleic Acid
Sequences

| | | | |
|---|---|---|---|
| | VL | | SEQ ID |
| Ab ID. | Group | VL Nucleic Acid Sequence | NO: |

29G4 variants

| | | | |
|---|---|---|---|
| 29G4v10 | LV-01 | GAGATCGTACTTACTCAGTCACCCGCCACATTGTCCCT | 328 |
| | | GAGCCCGGGTGAACGGGCGACCCTCAGCTGCCGAGCA | |
| | | TCCCAGTCCGTCGGACGATCATTGCACTGGTACCAACA | |
| | | AAAACCGGGCCAGGCCCCCAGACTTCTGATCAAGTAT | |
| | | GCGTCACAGAGCTTGTCGGGTATTCCCGCTCGCTTTTC | |
| | | GGGGTCGGGATCCGGGACAGATTTCACGCTCACAATC | |
| | | TCCTCGCTGGAACCCGAGGACTTCGCGGTCTACTATTG | |
| | | TCATCAGTCATCGAGGTTGCCTTTCACGTTTGGACCAG | |
| | | GGACCAAGGTGGACATTAAGCGTAC | |
| 29G4v22 | LV-02 | GATATCCAGCTCACTCAATCGCCATCATTTCTCTCCGC | 329 |
| | | TTCGGTAGGCGACCGGGTCACGATCACATGCAGGGCG | |
| | | TCGCAAAGCATTGGGAGGTCGTTGCATTGGTATCAGC | |
| | | AGAAACCCGGAAAGGCCCCGAAACTTCTGATCAAATA | |
| | | CGCATCACAAAGCTTGAGCGGTGTGCCGTCGCGCTTCT | |
| | | CCGGTTCCGGAAGCGGAACGGAATTCACGCTTACAAT | |
| | | CTCCTCACTGCAGCCCGAGGATTTCGCGACCTATTACT | |
| | | GTCACCAGTCATCCAGACTCCCGTTTACTTTTGGCCCT | |
| | | GGGACCAAGGTGGACATTAAGCGTAC | |
| iPS:420649; iPS:420653; | LV-03 | GAGATCGTACTTACTCAGTCACCCGCCACATTGTCCCT | 330 |
| iPS:420657; iPS:420661; | | GAGCCCGGGTGAACGGGCGACCCTCAGCTGCCGAGCA | |
| iPS:420665; iPS:420672; | | TCCAAGTCCGTCGGACGATCATTGCACTGGTACCAACA | |
| iPS:420679; iPS:420686; | | AAAACCGGGCCAGGCCCCCAGACTTCTGATCAAGTAT | |
| iPS:420837; iPS:420841; | | GCGTCACAGAGCTTGTCGGGTATTCCCGCTCGCTTTTC | |
| iPS:420845; iPS:420849; | | GGGGTCGGGATCCGGGACAGATTTCACGCTCACAATC | |
| iPS:420853; iPS:420857; | | TCCTCGCTGGAACCCGAGGACTTCGCGGTCTACTATTG | |
| iPS:420861; iPS:420865; | | TCATCAGTCATCGAGGTTGCCTTTCACGTTTGGACCAG | |
| iPS:420869; iPS:420873; | | GGACCAAGGTGGACATTAAG | |
| iPS:420877; iPS:420881; | | | |
| iPS:420885; iPS:420889; | | | |
| iPS:420893; iPS:420897; | | | |
| iPS:421027; iPS:421031; | | | |
| iPS:421035; iPS:421039; | | | |
| iPS:421043; iPS:421047; | | | |
| iPS:421051; iPS:421055; | | | |
| iPS:421059; iPS:421063; | | | |
| iPS:421067; iPS:421071; | | | |
| iPS:421075; iPS:421079; | | | |
| iPS:421083; iPS:421087; | | | |
| iPS:421147; iPS:421207; | | | |
| iPS:421211; iPS:421215; | | | |
| iPS:421219; iPS:421223 | | | |
| iPS:420690; iPS:420697; | LV-04 | GAGATCGTACTTACTCAGTCACCCGCCACATTGTCCCT | 331 |
| iPS:420704; iPS:420711; | | GAGCCCGGGTGAACGGGCGACCCTCAGCTGCCGAGCA | |
| iPS:420718; iPS:420725; | | TCCCAGTCCGTCGGACGATCATTGCACTGGTACCAACA | |
| iPS:420732; iPS:420739; | | AAAACCGGGCCAGGCCCCCAGACTTCTGATCAAGTAT | |
| iPS:420746; iPS:420753; | | GCGTCACAGAGCTTGTCGGGTATTCCCGCTCGCTTTTC | |
| iPS:420760; iPS:420767; | | GGGGTCGGGATCCGGGACAGATTTCACGCTCACAATC | |
| iPS:420774; iPS:420781; | | TCCTCGCTGGAACCCGAGGACTTCGCGGTCTACTATTG | |
| iPS:420788; iPS:420795; | | TCATCAGTCATCGAGGTTGCCTTTCACGTTTGGACCAG | |
| iPS:420802; iPS:420809; | | GGACCAAGGTGGACATTAAG | |
| iPS:420816; iPS:420823; | | | |
| iPS:420830; iPS:420901; | | | |
| iPS:420908; iPS:420915; | | | |
| iPS:420922; iPS:420929; | | | |
| iPS:420936; iPS:420943; | | | |
| iPS:420950; iPS:420957; | | | |
| iPS:420964; iPS:420971; | | | |
| iPS:420978; iPS:420985; | | | |
| iPS:420992; iPS:420999; | | | |
| iPS:421006; iPS:421013; | | | |
| iPS:421020; iPS:421091; | | | |
| iPS:421098; iPS:421105; | | | |
| iPS:421112; iPS:421119; | | | |
| iPS:421126; iPS:421133; | | | |
| iPS:421140; iPS:421163; | | | |
| iPS:391578; iPS:421170; | | | |
| iPS:421176; iPS:421182; | | | |
| iPS:421239; iPS:421246; | | | |
| iPS:421253; iPS:421260; | | | |
| iPS:421267; iPS:421286; | | | |
| iPS:421293; iPS:421300; | | | |
| iPS:421307; iPS:421326; | | | |
| iPS:421333; iPS:421340; | | | |
| iPS:421347; iPS:421354; | | | |
| iPS:421373; iPS:421380; | | | |
| iPS:421387; iPS:421394; | | | |
| iPS:421855; iPS:421861; | | | |
| iPS:421867; iPS:421873; | | | |
| iPS:421879; iPS:421885; | | | |
| iPS:421891; iPS:421897; | | | |
| iPS:421903; iPS:421909; | | | |
| iPS:421915 | | | |
| iPS:421151; iPS:421227; | LV-05 | GAGATCGTACTTACTCAGTCACCCGCCACATTGTCCCT | 332 |
| iPS:421274; iPS:421314; | | GAGCCCGGGTGAACGGGCGACCCTCAGCTGCCGAGCA | |
| iPS:421361 | | TCCCAGTCCGTCTGGCGATCATTGCACTGGTACCAACA | |
| | | AAAACCGGGCCAGGCCCCCAGACTTCTGATCAAGTAT | |
| | | GCGTCACAGAGCTTGTCGGGTATTCCCGCTCGCTTTTC | |
| | | GGGGTCGGGATCCGGGACAGATTTCACGCTCACAATC | |
| | | TCCTCGCTGGAACCCGAGGACTTCGCGGTCTACTATTG | |
| | | TCATCAGTCATCGAGGTTGCCTTTCACGTTTGGACCAG | |
| | | GGACCAAGGTGGACATTAAG | |
| iPS:391478; iPS:421231; | LV-06 | GAGATCGTACTTACTCAGTCACCCGCCACATTGTCCCT | 333 |
| iPS:421278; iPS:421318; | | GAGCCCGGGTGAACGGGCGACCCTCAGCTGCCGAGCA | |
| iPS:421365 | | TCCCAGTCCGTCGGACGAAACTTGCACTGGTACCAAC | |
| | | AAAAACCGGGCCAGGCCCCCAGACTTCTGATCAAGTA | |
| | | TGCGTCACAGAGCTTGTCGGGTATTCCCGCTCGCTTTT | |
| | | CGGGGTCGGGATCCGGGACAGATTTCACGCTCACAAT | |
| | | CTCCTCGCTGGAACCCGAGGACTTCGCGGTCTACTATT | |
| | | GTCATCAGTCATCGAGGTTGCCTTTCACGTTTGGACCA | |
| | | GGGACCAAGGTGGACATTAAG | |
| iPS:421157; iPS:421235; | LV-07 | GAGATCGTACTTACTCAGTCACCCGCCACATTGTCCCT | 334 |
| iPS:421282; iPS:421322; | | GAGCCCGGGTGAACGGGCGACCCTCAGCTGCCGAGCA | |
| iPS:421369 | | TCCCAGTCCGTCGGACGATCATTGCACTGGTACCAACA | |
| | | AAAACCGGGCCAGGCCCCCAGACTTCTGATCAAGTAT | |
| | | GCGTCACAGAGCTTGTCGGGTATTCCCGCTCGCTTTTC | |
| | | GGGGTCGGGATCCGGGACAGATTTCACGCTCACAATC | |
| | | TCCTCGCTGGAACCCGAGGACTTCGCGGTCTACTATTG | |
| | | TCATCAGTCATCGATGTTGCCTTTCACGTTTGGACCAG | |
| | | GGACCAAGGTGGACATTAAG | |
| iPS:421189 | LV-08 | GAGATCGTACTTACTCAGTCACCCGCCACATTGTCCCT | 335 |
| | | GAGCCCGGGTGAACGGGCGACCCTCAGCTGCCGAGCA | |
| | | TCCAAGTCCGTCTGGCGATCATTGCACTGGTACCAACA | |
| | | AAAACCGGGCCAGGCCCCCAGACTTCTGATCAAGTAT | |
| | | GCGTCACAGAGCTTGTCGGGTATTCCCGCTCGCTTTTC | |
| | | GGGGTCGGGATCCGGGACAGATTTCACGCTCACAATC | |
| | | TCCTCGCTGGAACCCGAGGACTTCGCGGTCTACTATTG | |
| | | TCATCAGTCATCGAGGTTGCCTTTCACGTTTGGACCAG | |
| | | GGACCAAGGTGGACATTAAG | |
| iPS:421195 | LV-09 | GAGATCGTACTTACTCAGTCACCCGCCACATTGTCCCT | 336 |
| | | GAGCCCGGGTGAACGGGCGACCCTCAGCTGCCGAGCA | |
| | | TCCAAGTCCGTCGGACGAAACTTGCACTGGTACCAAC | |
| | | AAAAACCGGGCCAGGCCCCCAGACTTCTGATCAAGTA | |
| | | TGCGTCACAGAGCTTGTCGGGTATTCCCGCTCGCTTTT | |
| | | CGGGGTCGGGATCCGGGACAGATTTCACGCTCACAAT | |
| | | CTCCTCGCTGGAACCCGAGGACTTCGCGGTCTACTATT | |
| | | GTCATCAGTCATCGAGGTTGCCTTTCACGTTTGGACCA | |
| | | GGGACCAAGGTGGACATTAAG | |
| iPS:421201 | LV-10 | GAGATCGTACTTACTCAGTCACCCGCCACATTGTCCCT | 337 |
| | | GAGCCCGGGTGAACGGGCGACCCTCAGCTGCCGAGCA | |
| | | TCCAAGTCCGTCGGACGATCATTGCACTGGTACCAACA | |
| | | AAAACCGGGCCAGGCCCCCAGACTTCTGATCAAGTAT | |
| | | GCGTCACAGAGCTTGTCGGGTATTCCCGCTCGCTTTTC | |
| | | GGGGTCGGGATCCGGGACAGATTTCACGCTCACAATC | |
| | | TCCTCGCTGGAACCCGAGGACTTCGCGGTCTACTATTG | |
| | | TCATCAGTCATCGATGTTGCCTTTCACGTTTGGACCAG | |
| | | GGACCAAGGTGGACATTAAG | |
| iPS:480711; iPS:480706; | LV-11 | GAGATCGTACTTACTCAGTCACCCGCCACATTGTCCCT | 338 |
| iPS:480705; iPS:480707; | | GAGCCCGGGTGAACGGGCGACCCTCAGCTGCCGAGCA | |
| iPS:480708; iPS:480712; | | TCCAAATCCGTCGGGTGGAGCTTGCACTGGTACCAAC | |
| iPS:480704; iPS:480710 | | AAAAACCGGGCCAGGCCCCCAGACTTCTGATCAAGTA | |
| | | TGCGTCACAGAGCTTGTCGGGTATTCCCGCTCGCTTTT | |
| | | CGGGGTCGGGATCCGGGACAGATTTCACGCTCACAAT | |
| | | CTCCTCGCTGGAACCCGAGGACTTCGCGGTCTACTATT | |
| | | GTCATCAGTCATCGAGGTTGCCTTTCACGTTTGGACCA | |
| | | GGGACCAAGGTGGACATTAAGCGTA | |
| iPS:480713 | LV-12 | GAGATCGTACTTACTCAGTCACCCGCCACATTGTCCCT | 339 |
| | | GAGCCCGGGTGAACGGGCGACCCTCAGCTGCCGAGCA | |
| | | TCCAAATCCGTCGGGTACAGCTTGCACTGGTACCAACA | |
| | | AAAACCGGGCCAGGCCCCCAGACTTCTGATCAAGTAT | |
| | | GCGTCACAGAGCTTGTCGGGTATTCCCGCTCGCTTTTC | |
| | | GGGGTCGGGATCCGGGACAGATTTCACGCTCACAATC | |
| | | TCCTCGCTGGAACCCGAGGACTTCGCGGTCTACTATTG | |
| | | TCATCAGTCATCGAGGTTGCCTTTCACGTTTGGACCAG | |
| | | GGACCAAGGTGGACATTAAGCGTA | |
| iPS:480709 | LV-13 | GAGATCGTACTTACTCAGTCACCCGCCACATTGTCCCT | 340 |
| | | GAGCCCGGGTGAACGGGCGACCCTCAGCTGCCGAGCA | |
| | | TCCAAAGCCGTCGGGTGGAGCTTGCACTGGTACCAAC | |
| | | AAAAACCGGGCCAGGCCCCCAGACTTCTGATCAAGTA | |
| | | TGCGTCACAGAGCTTGTCGGGTATTCCCGCTCGCTTTT | |
| | | CGGGGTCGGGATCCGGGACAGATTTCACGCTCACAAT | |
| | | CTCCTCGCTGGAACCCGAGGACTTCGCGGTCTACTATT | |
| | | GTCATCAGTCATCGAGGTTGCCTTTCACGTTTGGACCA | |
| | | GGGACCAAGGTGGACATTAAGCGTA | |
| iPS:480716; iPS:480715 | LV-14 | GAGATCGTACTTACTCAGTCACCCGCCACATTGTCCCT | 341 |
| iPS:480717 | | GAGCCCGGGTGAACGGGCGACCCTCAGCTGCCGAGCA | |
| | | TCCAAATCAGTCGGTCAGTCTTTGCACTGGTACCAACA | |
| | | AAAACCGGGCCAGGCCCCCAGACTTCTGATCAAGTAT | |
| | | GCGTCACAGAGCTTGTCGGGTATTCCCGCTCGCTTTTC | |
| | | GGGGTCGGGATCCGGGACAGATTTCACGCTCACAATC | |
| | | TCCTCGCTGGAACCCGAGGACTTCGCGGTCTACTATTG | |
| | | TCATCAGTCATCGCGTTTGCCTTTCACGTTTGGACCAG | |
| | | GGACCAAGGTGGACATTAAGCGTA | |
| iPS:480714 | LV-15 | GAGATCGTACTTACTCAGTCACCCGCCACATTGTCCCT | 342 |
| | | GAGCCCGGGTGAACGGGCGACCCTCAGCTGCCGAGCA | |
| | | TCCCGTTCAGTCGGTCTGGCTTTGCACTGGTACCAACA | |
| | | AAAACCGGGCCAGGCCCCCAGACTTCTGATCAAGTAT | |
| | | GCGTCACAGAGCTTGTCGGGTATTCCCGCTCGCTTTTC | |
| | | GGGGTCGGGATCCGGGACAGATTTCACGCTCACAATC | |
| | | TCCTCGCTGGAACCCGAGGACTTCGCGGTCTACTATTG | |
| | | TCATCAGTCATCGTTCTTGCCTTTCACGTTTGGACCAG | |
| | | GGACCAAGGTGGACATTAAGCGTA | |

19H8 variants

| | | | |
|---|---|---|---|
| 19H8 | LV-16 | GACATCCAGATGACCCAGTCTCCATCCTCCCTGTCTGC | 343 |
| | | ATCTGTAGGAGACAGAATCACCATCACTTGCCGGGCA | |
| | | AGTCAGAGCATTAGCAGGTATTTAAATTGGTATCAAC | |
| | | AGAAACCAGGGAAAGCCCCTAAACTCCTGATCTATGC | |
| | | TGCATCCAGTTTGCAAAGTGGGATCCCATCAAGGTTCA | |
| | | GCGGCAGTGGATCTGGGACAGATTTCACTCTCACCATC | |
| | | AACAGTCTGCAACCTGAAGATTTTGCAACTTACTTCTG | |
| | | TCAACAGAGTTACAGTCCCCCATTCACTTTCGGCCCTG | |
| | | GGACCAAAGTGGATATCAAACGTAC | |
| iPS:448202 | LV-17 | GACATCCAGATGACCCAGTCTCCATCCTCCCTGTCTGC | 344 |
| | | ATCTGTAGGAGACAGAATCACCATCACTTGCCGGGCA | |
| | | AGTCAGAGCATTAGCAGGTATTTAAATTGGTATCAAC | |
| | | AGAAACCAGGGAAAGCCCCTAAACTCCTGATCTTCGC | |
| | | TGGTCAGCGTTTGCAAAGTGGGATCCCATCAAGGTTCA | |
| | | GCGGCAGTGGATCTGGGACAGATTTCACTCTCACCATC | |
| | | AACAGTCTGCAACCTGAAGATTTTGCAACTTACTTCTG | |
| | | TCAACAGGCTATCGGTATGCCATACACTTTCGGCCCTG | |
| | | GGACCAAAGTGGATATCAAA | |
| iPS:449375 | LV-18 | GACATCCAGATGACCCAGTCTCCATCCTCCCTGTCTGC | 345 |
| | | ATCTGTAGGAGACAGAATCACCATCACTTGCCGGGCA | |
| | | AGTCAGTACATTGTTCGTTACTTAAACTGGTATCAACA | |
| | | GAAACCAGGGAAAGCCCCTAAACTCCTGATCTACGCT | |
| | | GCTCATCATTTGCAAAGTGGGATCCCATCAAGGTTCAG | |
| | | CGGCAGTGGATCTGGGACAGATTTCACTCTCACCATCA | |
| | | ACAGTCTGCAACCTGAAGATTTTGCAACTTACTTCTGT | |
| | | CAACAGGCTATCCAGGAACCATACACTTTCGGCCCTG | |
| | | GGACCAAAGTGGATATCAAA | |
| iPS:448083 | LV-19 | GACATCCAGATGACCCAGTCTCCATCCTCCCTGTCTGC | 346 |
| | | ATCTGTAGGAGACAGAATCACCATCACTTGCCGGGCA | |
| | | AGTCAGACTATTGTTCGTTACTTAAACTGGTATCAACA | |
| | | GAAACCAGGGAAAGCCCCTAAACTCCTGATCTTCGCT | |
| | | GGTCAGCGTTTGCAAAGTGGGATCCCATCAAGGTTCA | |
| | | GCGGCAGTGGATCTGGGACAGATTTCACTCTCACCATC | |
| | | AACAGTCTGCAACCTGAAGATTTTGCAACTTACTTCTG | |
| | | TCAACAGGCTATCATCAACCCATACACTTTCGGCCCTG | |
| | | GGACCAAAGTGGATATCAAA | |
| iPS:452128 | LV-20 | GACATCCAGATGACCCAGTCTCCATCCTCCCTGTCTGC | 347 |
| | | ATCTGTAGGAGACAGAATCACCATCACTTGCCGGGCA | |
| | | AGTCAGTACATTGTTCGTTACTTAAACTGGTATCAACA | |
| | | GAAACCAGGGAAAGCCCCTAAACTCCTGATCTACGCT | |
| | | GCTAACATGTTGCAAAGTGGGATCCCATCAAGGTTCA | |
| | | GCGGCAGTGGATCTGGGACAGATTTCACTCTCACCATC | |
| | | AACAGTCTGCAACCTGAAGATTTTGCAACTTACTTCTG | |
| | | TCAACAGGCTATCAACCAGCCATACACTTTCGGCCCTG | |
| | | GGACCAAAGTGGATATCAAA | |
| iPS:448195 | LV-21 | GACATCCAGATGACCCAGTCTCCATCCTCCCTGTCTGC | 348 |
| | | ATCTGTAGGAGACAGAATCACCATCACTTGCCGGGCA | |
| | | AGTCAGAAAATTGCTCGTTACTTAGTTTGGTATCAACA | |
| | | GAAACCAGGGAAAGCCCCTAAACTCCTGATCTACGCT | |
| | | GCTAACATGTTGCAAAGTGGGATCCCATCAAGGTTCA | |
| | | GCGGCAGTGGATCTGGGACAGATTTCACTCTCACCATC | |
| | | AACAGTCTGCAACCTGAAGATTTTGCAACTTACTTCTG | |
| | | TCAACAGTCTATCCAGCAGCCATACACTTTCGGCCCTG | |
| | | GGACCAAAGTGGATATCAAA | |
| iPS:448466 | LV-22 | GACATCCAGATGACCCAGTCTCCATCCTCCCTGTCTGC | 349 |
| | | ATCTGTAGGAGACAGAATCACCATCACTTGCCGGGCA | |
| | | AGTCAGAGCATTAGCAGGTATTTAAATTGGTATCAAC | |
| | | AGAAACCAGGGAAAGCCCCTAAACTCCTGATCTTCGC | |
| | | TGGTCAGCGTTTGCAAAGTGGGATCCCATCAAGGTTCA | |
| | | GCGGCAGTGGATCTGGGACAGATTTCACTCTCACCATC | |
| | | AACAGTCTGCAACCTGAAGATTTTGCAACTTACTTCTG | |
| | | TCAACAGGCTATCCAGCAGCCATACACTTTCGGCCCTG | |
| | | GGACCAAAGTGGATATCAAACGT | |
| iPS:448689 | LV-23 | GACATCCAGATGACCCAGTCTCCATCCTCCCTGTCTGC | 350 |
| | | ATCTGTAGGAGACAGAATCACCATCACTTGCCGGGCA | |
| | | AGTCAGTACATTGTTCGTTACTTAAACTGGTATCAACA | |
| | | GAAACCAGGGAAAGCCCCTAAACTCCTGATCTACGCT | |
| | | TCTTACAACTTGCAAAGTGGGATCCCATCAAGGTTCAG | |
| | | CGGCAGTGGATCTGGGACAGATTTCACTCTCACCATCA | |
| | | ACAGTCTGCAACCTGAAGATTTTGCAACTTACTTCTGT | |
| | | CAACAGGCTATCATGGCTCCATACACTTTCGGCCCTGG | |
| | | GACCAAAGTGGATATCAAA | |
| iPS:449034 | LV-24 | GACATCCAGATGACCCAGTCTCCATCCTCCCTGTCTGC | 351 |
| | | ATCTGTAGGAGACAGAATCACCATCACTTGCCGGGCA | |
| | | AGTCAGCCTATTGCTCAGTACTTAAACTGGTATCAACA | |
| | | GAAACCAGGGAAAGCCCCTAAACTCCTGATCTACGCT | |
| | | GGTCGTTACTTGCAAAGTGGGATCCCATCAAGGTTCAG | |
| | | CGGCAGTGGATCTGGGACAGATTTCACTCTCACCATCA | |
| | | ACAGTCTGCAACCTGAAGATTTTGCAACTTACTTCTGT | |
| | | CAACAGGCTATCCAGAACCCATACACTTTCGGCCCTGG | |
| | | GACCAAAGTGGATATCAAA | |
| iPS:448075 | LV-25 | GACATCCAGATGACCCAGTCTCCATCCTCCCTGTCTGC | 352 |
| | | ATCTGTAGGAGACAGAATCACCATCACTTGCCGGGCA | |
| | | AGTCAGAGCATTAGCAGGTATTTAAATTGGTATCAAC | |
| | | AGAAACCAGGGAAAGCCCCTAAACTCCTGATCTTCGC | |
| | | TGGTCAGCGTTTGCAAAGTGGGATCCCATCAAGGTTCA | |
| | | GCGGCAGTGGATCTGGGACAGATTTCACTCTCACCATC | |
| | | AACAGTCTGCAACCTGAAGATTTTGCAACTTACTTCTG | |
| | | TCAACAGGCTATCGTTCAGCCATACACTTTCGGCCCTG | |
| | | GGACCAAAGTGGATATCAAA | |
| iPS:448924 | LV-26 | GACATCCAGATGACCCAGTCTCCATCCTCCCTGTCTGC | 353 |
| | | ATCTGTAGGAGACAGAATCACCATCACTTGCCGGGCA | |
| | | AGTCAGCCGATTTCTCGTTACTTATCTTGGTATCAACA | |
| | | GAAACCAGGGAAAGCCCCTAAACTCCTGATCTTCGCT | |
| | | GGTCAGCGTTTGCAAAGTGGGATCCCATCAAGGTTCA | |
| | | GCGGCAGTGGATCTGGGACAGATTTCACTCTCACCATC | |
| | | AACAGTCTGCAACCTGAAGATTTTGCAACTTACTTCTG | |
| | | TCAACAGGCTATCTCTATCCCATACACTTTCGGCCCTG | |
| | | GGACCAAAGTGGATATCAAA | |
| iPS:448752; 3575 | LV-27 | GACATCCAGATGACCCAGTCTCCATCCTCCCTGTCTGC | 354 |
| | | ATCTGTAGGAGACAGAATCACCATCACTTGCCGGGCA | |
| | | AGTCAGCAGATTGCTCGTTACTTAAACTGGTATCAACA | |
| | | GAAACCAGGGAAAGCCCCTAAACTCCTGATCTACGCT | |
| | | TCTTACAACTTGCAAAGTGGGATCCCATCAAGGTTCAG | |
| | | CGGCAGTGGATCTGGGACAGATTTCACTCTCACCATCA | |
| | | ACAGTCTGCAACCTGAAGATTTTGCAACTTACTTCTGT | |
| | | CAACAGGCTATCATCCAGCCATACACTTTCGGCCCTGG | |
| | | GACCAAAGTGGATATCAAA | |
| iPS:448772; iPS:448593 | LV-28 | GACATCCAGATGACCCAGTCTCCATCCTCCCTGTCTGC | 355 |
| | | ATCTGTAGGAGACAGAATCACCATCACTTGCCGGGCA | |
| | | AGTCAGAGCATTAGCAGGTATTTAAATTGGTATCAAC | |
| | | AGAAACCAGGGAAAGCCCCTAAACTCCTGATCTACGC | |
| | | TTCTTACAACTTGCAAAGTGGGATCCCATCAAGGTTCA | |
| | | GCGGCAGTGGATCTGGGACAGATTTCACTCTCACCATC | |
| | | AACAGTCTGCAACCTGAAGATTTTGCAACTTACTTCTG | |
| | | TCAACAGGCTATCCAGAACCCATACACTTTCGGCCCTG | |
| | | GGACCAAAGTGGATATCAAA | |
| iPS:448117 | LV-29 | GACATCCAGATGACCCAGTCTCCATCCTCCCTGTCTGC | 356 |
| | | ATCTGTAGGAGACAGAATCACCATCACTTGCCGGGCA | |
| | | AGTCAGACTATTGTTCGTTACTTAAACTGGTATCAACA | |
| | | GAAACCAGGGAAAGCCCCTAAACTCCTGATCTTCGCT | |
| | | GGTCAGCGTTTGCAAAGTGGGATCCCATCAAGGTTCA | |
| | | GCGGCAGTGGATCTGGGACAGATTTCACTCTCACCATC | |
| | | AACAGTCTGCAACCTGAAGATTTTGCAACTTACTTCTG | |
| | | TCAACAGTCTATCCAGACTCCATACACTTTCGGCCCTG | |
| | | GGACCAAAGTGGATATCAAA | |
| iPS:448788 | LV-30 | GACATCCAGATGACCCAGTCTCCATCCTCCCTGTCTGC | 357 |
| | | ATCTGTAGGAGACAGAATCACCATCACTTGCCGGGCA | |
| | | AGTCAGAGCATTAGCAGGTATTTAAATTGGTATCAAC | |
| | | AGAAACCAGGGAAAGCCCCTAAACTCCTGATCTACGC | |
| | | TGGTCGTATCTTGCAAAGTGGGATCCCATCAAGGTTCA | |
| | | GCGGCAGTGGATCTGGGACAGATTTCACTCTCACCATC | |
| | | AACAGTCTGCAACCTGAAGATTTTGCAACTTACTTCTG | |
| | | TCAACAGGCTATCATCAACCCATACACTTTCGGCCCTG | |
| | | GGACCAAAGTGGATATCAAA | |
| iPS:448238 | LV-31 | GACATCCAGATGACCCAGTCTCCATCCTCCCTGTCTGC | 358 |
| | | ATCTGTAGGAGACAGAATCACCATCACTTGCCGGGCA | |
| | | AGTCAGCGTATTGCTCGTTACTTAAACTGGTATCAACA | |
| | | GAAACCAGGGAAAGCCCCTAAACTCCTGATCTTCGCT | |
| | | GGTTCTATCTTGCAAAGTGGGATCCCATCAAGGTTCAG | |
| | | CGGCAGTGGATCTGGGACAGATTTCACTCTCACCATCA | |
| | | ACAGTCTGCAACCTGAAGATTTTGCAACTTACTTCTGT | |
| | | CAACAGGCTATCCAGAACCCATACACTTTCGGCCCTGG | |
| | | GACCAAAGTGGATATCAAA | |
| iPS:448901 | LV-32 | GACATCCAGATGACCCAGTCTCCATCCTCCCTGTCTGC | 359 |
| | | ATCTGTAGGAGACAGAATCACCATCACTTGCCGGGCA | |
| | | AGTCAGAGCATTAGCAGGTATTTAAATTGGTATCAAC | |
| | | AGAAACCAGGGAAAGCCCCTAAACTCCTGATCTACGC | |
| | | TTCTTACAACTTGCAAAGTGGGATCCCATCAAGGTTCA | |
| | | GCGGCAGTGGATCTGGGACAGATTTCACTCTCACCATC | |
| | | AACAGTCTGCAACCTGAAGATTTTGCAACTTACTTCTG | |
| | | TCAACAGTCTATCCAGCAGCCATACACTTTCGGCCCTG | |
| | | GGACCAAAGTGGATATCAAA | |
| iPS:448655 | LV-33 | GACATCCAGATGACCCAGTCTCCATCCTCCCTGTCTGC | 360 |
| | | ATCTGTAGGAGACAGAATCACCATCACTTGCCGGGCA | |
| | | AGTCAGTACATTGTTCGTTACTTAAACTGGTATCAACA | |
| | | GAAACCAGGGAAAGCCCCTAAACTCCTGATCTACGCT | |
| | | TCTTACAACTTGCAAAGTGGGATCCCATCAAGGTTCAG | |
| | | CGGCAGTGGATCTGGGACAGATTTCACTCTCACCATCA | |
| | | ACAGTCTGCAACCTGAAGATTTTGCAACTTACTTCTGT | |
| | | CAACAGGCTATCCAGCAGCCATACACTTTCGGCCCTGG | |
| | | GACCAAAGTGGATATCAAA | |
| iPS:448730 | LV-34 | GACATCCAGATGACCCAGTCTCCATCCTCCCTGTCTGC | 361 |
| | | ATCTGTAGGAGACAGAATCACCATCACTTGCCGGGCA | |
| | | AGTCAGATGATTGCTCGTTACTTAAACTGGTATCAACA | |
| | | GAAACCAGGGAAAGCCCCTAAACTCCTGATCTACGCT | |
| | | TCTTACAACTTGCAAAGTGGGATCCCATCAAGGTTCAG | |
| | | CGGCAGTGGATCTGGGACAGATTTCACTCTCACCATCA | |
| | | ACAGTCTGCAACCTGAAGATTTTGCAACTTACTTCTGT | |
| | | CAACAGGCTATCATCAACCCATACACTTTCGGCCCTGG | |
| | | GACCAAAGTGGATATCAAA | |
| iPS:449027 | LV-35 | GACATCCAGATGACCCAGTCTCCATCCTCCCTGTCTGC | 362 |
| | | ATCTGTAGGAGACAGAATCACCATCACTTGCCGGGCA | |
| | | AGTCAGTACATTGTTCGTTACTTAAACTGGTATCAACA | |
| | | GAAACCAGGGAAAGCCCCTAAACTCCTGATCTACGGT | |
| | | GCTCGTAACTTGCAAAGTGGGATCCCATCAAGGTTCA | |
| | | GCGGCAGTGGATCTGGGACAGATTTCACTCTCACCATC | |
| | | AACAGTCTGCAACCTGAAGATTTTGCAACTTACTTCTG | |
| | | TCAACAGTCTATCCAGACTCCATACACTTTCGGCCCTG | |
| | | GGACCAAAGTGGATATCAAA | |
| 3574 | LV-36 | GACATCCAGATGACCCAGTCTCCATCCTCCCTGTCTGC | 363 |
| | | ATCTGTAGGAGACAGAATCACCATCACTTGCCGGGCA | |
| | | AGTCAGAGCATTAGCAGGTATTTAAATTGGTATCAAC | |
| | | AGAAACCAGGGAAAGCCCCTAAACTCCTGATCTATGC | |
| | | TGCATCCAGTTTGCAAAGTGGGATCCCATCAAGGTTCA | |
| | | GCGGCAGTGGATCTGGGACAGATTTCACTCTCACCATC | |
| | | AACAGTCTGCAACCTGAAGATTTTGCAACTTACTTCTG | |
| | | TCAACAGAGTTACAGTCCCCCATTCACTTTCGGCCCTG | |
| | | GGACCAAAGTGGATATCAAA | |

TABLE 5B
Exemplary Anti-PAC1 Antibody Heavy Chain Variable Region
Nucleic Acid Sequences

| | | | |
|---|---|---|---|
| | VH | | SEQ ID |
| Ab ID. | Group | VH Nucleic Acid Sequence | NO: |

29G4 variants

| | | | |
|---|---|---|---|
| 29G4v10; | HV-01 | CAGGTGCAGCTGGTGGAGTCTGGGGGAGGCGTGGTCCAGCCTGG | 364 |
| iPS:421151; | | GAGGTCCCTGCGACTCTCCTGTGCAGCCTCTGGATTCACCTTCAG | |
| iPS:391478; | | TAGATTTGCCATGCACTGGGTCCGCCAGGCTCCAGGCAAGGGGCT | |
| iPS:421157; | | GGAGTGGGTGGCAGTTATATCATATGATGGAGGAAATAAATACT | |
| iPS:421189; | | ATGCAGAGTCCGTGAAGGGCCGGTTCACCATCTCCAGAGACAATT | |
| iPS:421195; | | CCAAGAACACCCTGTATCTGCAAATGAACAGCCTGAGAGCTGAG | |
| iPS:421201 | | GACACGGCTCTGTTTTACTGTGCGAGAGGATACGATGTTTTGACT | |
| | | GGTTACCCCGACTACTGGGGCCAGGGAACCCTGGTCACCGTCTCC | |
| | | TCAGC | |
| 29G4v22 | HV-02 | CAAGTTCAGTTGGTGGAGTCTGGAGCCGAAGTAGTAAAGCCAGG | 365 |
| | | AGCTTCAGTGAAAGTCTCTTGTAAAGCAAGTGGATTCACGTTTAG | |
| | | CCGCTTTGCCATGCATTGGGTGCGGCAAGCTCCCGGTCAGGGGTT | |
| | | GGAGTGGATGGGAGTTATTAGCTATGACGGGGGCAATAAGTACT | |
| | | ACGCCGAGTCTGTTAAGGGTCGGGTCACAATGACACGGGACACCT | |
| | | CAACCAGTACACTCTATATGGAACTGTCTAGCCTGAGATCCGAGG | |
| | | ACACCGCTGTGTATTATTGCGCTAGGGGGTACGATGTATTGACGG | |
| | | GTTATCCTGATTACTGGGGGCAGGGGACACTCGTAACCGTCTCTA | |
| | | GT | |
| iPS:420649; | HV-03 | CAGGTGCAGCTGGTGGAGTCTGGGGGAGGCGTGGTCCAGCCTGG | 366 |
| iPS:421227; | | GAGGTCCCTGCGACTCTCCTGTGCAGCCTCTGGATTCACCTTCAG | |
| iPS:421231; | | TAGATTTGCCATGCACTGGGTCCGCCAGGCTCCAGGCAAGGGGCT | |
| iPS:421235 | | GGAGTGGGTGGCAGTTATATCATATAACGGAGGAAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATGTTTTGACT | |
| | | GGTTACCCCGACTACTGGGGCCAGGGAACCCTGGTCACCGTCTCT | |
| | | AGT | |
| iPS:420653 | HV-04 | CAGGTGCAGCTGGTGGAGTCTGGGGGAGGCGTGGTCCAGCCTGG | 367 |
| | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATATCGGAGGAAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATGTTTTGACT | |
| | | GGTTACCCCGACTACTGGGGCCAGGGAACCCTGGTCACCGTCTCT | |
| | | AGT | |
| iPS:420657 | HV-05 | CAGGTGCAGCTGGTGGAGTCTGGGGGAGGCGTGGTCCAGCCTGG | 368 |
| | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATCAGGGAGGAAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATGTTTTGACT | |
| | | GGTTACCCCGACTACTGGGGCCAGGGAACCCTGGTCACCGTCTCT | |
| | | AGT | |
| iPS:420661 | HV-06 | CAGGTGCAGCTGGTGGAGTCTGGGGGAGGCGTGGTCCAGCCTGG | 369 |
| | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATTACGGAGGAAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATGTTTTGACT | |
| | | GGTTACCCCGACTACTGGGGCCAGGGAACCCTGGTCACCGTCTCT | |
| | | AGT | |
| iPS:420665; | HV-07 | CAGGTGCAGCTGGTGGAGTCTGGGGGAGGCGTGGTCCAGCCTGG | 370 |
| iPS:421274; | | GAGGTCCCTGCGACTCTCCTGTGCAGCCTCTGGATTCACCTTCAG | |
| iPS:421278; | | TAGATTTGCCATGCACTGGGTCCGCCAGGCTCCAGGCAAGGGGCT | |
| iPS:421282 | | GGAGTGGGTGGCAGTTATATCATATGATGGACGCAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATGTTTTGACT | |
| | | GGTTACCCCGACTACTGGGGCCAGGGAACCCTGGTCACCGTCTCT | |
| | | AGT | |
| iPS:420672 | HV-08 | CAGGTGCAGCTGGTGGAGTCTGGGGGAGGCGTGGTCCAGCCTGG | 371 |
| | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATGATGGAAACAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATGTTTTGACT | |
| | | GGTTACCCCGACTACTGGGGCCAGGGAACCCTGGTCACCGTCTCT | |
| | | AGT | |
| iPS:420679; | HV-09 | CAGGTGCAGCTGGTGGAGTCTGGGGGAGGCGTGGTCCAGCCTGG | 372 |
| iPS:421314; | | GAGGTCCCTGCGACTCTCCTGTGCAGCCTCTGGATTCACCTTCAG | |
| iPS:421318; | | TAGATTTGCCATGCACTGGGTCCGCCAGGCTCCAGGCAAGGGGCT | |
| iPS:421322 | | GGAGTGGGTGGCAGTTATATCATATGATGGAGGAAATAAATACT | |
| | | ATGCACGCTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATGTTTTGACT | |
| | | GGTTACCCCGACTACTGGGGCCAGGGAACCCTGGTCACCGTCTCT | |
| | | AGT | |
| iPS:420686; | HV-10 | CAGGTGCAGCTGGTGGAGTCTGGGGGAGGCGTGGTCCAGCCTGG | 373 |
| iPS:421361; | | GAGGTCCCTGCGACTCTCCTGTGCAGCCTCTGGATTCACCTTCAG | |
| iPS:421365; | | TAGATTTGCCATGCACTGGGTCCGCCAGGCTCCAGGCAAGGGGCT | |
| iPS:421369 | | GGAGTGGGTGGCAGTTATATCATATGATGGAGGAAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATATCTTGACT | |
| | | GGTTACCCCGACTACTGGGGCCAGGGAACCCTGGTCACCGTCTCT | |
| | | AGT | |
| iPS:420690; | HV-11 | CAGGTGCAGCTGGTGGAGTCTGGGGGAGGCGTGGTCCAGCCTGG | 374 |
| iPS:420837 | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATAACGGACGCAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATGTTTTGACT | |
| | | GGTTACCCCGACTACTGGGGCCAGGGAACCCTGGTCACCGTCTCT | |
| | | AGT | |
| iPS:420697; | HV-12 | CAGGTGCAGCTGGTGGAGTCTGGGGGAGGCGTGGTCCAGCCTGG | 375 |
| iPS:420841 | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATATCGGACGCAATAAATACTA | |
| | | TGCAGAGTCCGTGAAGGGCCGGTTCACCATCTCCAGAGACAATTC | |
| | | CAAGAACACCCTGTATCTGCAAATGAACAGCCTGAGAGCTGAGG | |
| | | ACACGGCTCTGTTTTACTGTGCGAGAGGATACGATGTTTTGACTG | |
| | | GTTACCCCGACTACTGGGGCCAGGGAACCCTGGTCACCGTCTCTA | |
| | | GT | |
| iPS:420704; | HV-13 | CAGGTGCAGCTGGTGGAGTCTGGGGGAGGCGTGGTCCAGCCTGG | 376 |
| iPS:420845 | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATCAGGGACGCAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATGTTTTGACT | |
| | | GGTTACCCCGACTACTGGGGCCAGGGAACCCTGGTCACCGTCTCT | |
| | | AGT | |
| iPS:420711; | HV-14 | CAGGTGCAGCTGGTGGAGTCTGGGGGAGGCGTGGTCCAGCCTGG | 377 |
| iPS:420849 | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATTACGGACGCAATAAATACTA | |
| | | TGCAGAGTCCGTGAAGGGCCGGTTCACCATCTCCAGAGACAATTC | |
| | | CAAGAACACCCTGTATCTGCAAATGAACAGCCTGAGAGCTGAGG | |
| | | ACACGGCTCTGTTTTACTGTGCGAGAGGATACGATGTTTTGACTG | |
| | | GTTACCCCGACTACTGGGGCCAGGGAACCCTGGTCACCGTCTCTA | |
| | | GT | |
| iPS:420718; | HV-15 | CAGGTGCAGCTGGTGGAGTCTGGGGGAGGCGTGGTCCAGCCTGG | 378 |
| iPS:420853 | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATAACGGAAACAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATGTTTTGACT | |
| | | GGTTACCCCGACTACTGGGGCCAGGGAACCCTGGTCACCGTCTCT | |
| | | AGT | |
| iPS:420725; | HV-16 | CAGGTGCAGCTGGTGGAGTCTGGGGGAGGCGTGGTCCAGCCTGG | 379 |
| iPS:420857 | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATATCGGAAACAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATGTTTTGACT | |
| | | GGTTACCCCGACTACTGGGGCCAGGGAACCCTGGTCACCGTCTCT | |
| | | AGT | |
| iPS:420732; | HV-17 | CAGGTGCAGCTGGTGGAGTCTGGGGGAGGCGTGGTCCAGCCTGG | 380 |
| iPS:420861 | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATCAGGGAAACAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATGTTTTGACT | |
| | | GGTTACCCCGACTACTGGGGCCAGGGAACCCTGGTCACCGTCTCT | |
| | | AGT | |
| iPS:420739; | HV-18 | CAGGTGCAGCTGGTGGAGTCTGGGGGAGGCGTGGTCCAGCCTGG | 381 |
| iPS:420865 | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATTACGGAAACAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATGTTTTGACT | |
| | | GGTTACCCCGACTACTGGGGCCAGGGAACCCTGGTCACCGTCTCT | |
| | | AGT | |
| iPS:420746; | HV-19 | CAGGTGCAGCTGGTGGAGTCTGGGGGAGGCGTGGTCCAGCCTGG | 382 |
| iPS:420869 | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATAACGGAGGAAATAAATACT | |
| | | ATGCACGCTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATGTTTTGACT | |
| | | GGTTACCCCGACTACTGGGGCCAGGGAACCCTGGTCACCGTCTCT | |
| | | AGT | |
| iPS:420753; | HV-20 | CAGGTGCAGCTGGTGGAGTCTGGGGGAGGCGTGGTCCAGCCTGG | 383 |
| iPS:420873 | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATATCGGAGGAAATAAATACT | |
| | | ATGCACGCTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATGTTTTGACT | |
| | | GGTTACCCCGACTACTGGGGCCAGGGAACCCTGGTCACCGTCTCT | |
| | | AGT | |
| iPS:420760; | HV-21 | CAGGTGCAGCTGGTGGAGTCTGGGGGAGGCGTGGTCCAGCCTGG | 384 |
| iPS:420877 | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATCAGGGAGGAAATAAATACT | |
| | | ATGCACGCTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATGTTTTGACT | |
| | | GGTTACCCCGACTACTGGGGCCAGGGAACCCTGGTCACCGTCTCT | |
| | | AGT | |
| iPS:420767; | HV-22 | CAGGTGCAGCTGGTGGAGTCTGGGGGAGGCGTGGTCCAGCCTGG | 385 |
| iPS:420881 | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATTACGGAGGAAATAAATACT | |
| | | ATGCACGCTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATGTTTTGACT | |
| | | GGTTACCCCGACTACTGGGGCCAGGGAACCCTGGTCACCGTCTCT | |
| | | AGT | |
| iPS:420774; | HV-23 | CAGGTGCAGCTGGTGGAGTCTGGGGGAGGCGTGGTCCAGCCTGG | 386 |
| iPS:420885 | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATAACGGAGGAAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATATCTTGACT | |
| | | GGTTACCCCGACTACTGGGGCCAGGGAACCCTGGTCACCGTCTCT | |
| | | AGT | |
| iPS:420781; | HV-24 | CAGGTGCAGCTGGTGGAGTCTGGGGGAGGCGTGGTCCAGCCTGG | 387 |
| iPS:420889 | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATATCGGAGGAAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATATCTTGACT | |
| | | GGTTACCCCGACTACTGGGGCCAGGGAACCCTGGTCACCGTCTCT | |
| | | AGT | |
| iPS:420788; | HV-25 | CAGGTGCAGCTGGTGGAGTCTGGGGGAGGCGTGGTCCAGCCTGG | 388 |
| iPS:420893 | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATCAGGGAGGAAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATATCTTGACT | |
| | | GGTTACCCCGACTACTGGGGCCAGGGAACCCTGGTCACCGTCTCT | |
| iPS:420795; | HV-26 | CAGGTGCAGCTGGTGGAGTCTGGGGGAGGCGTGGTCCAGCCTGG | 389 |
| iPS:420897 | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATTACGGAGGAAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATATCTTGACT | |
| | | GGTTACCCCGACTACTGGGGCCAGGGAACCCTGGTCACCGTCTCT | |
| | | AGT | |
| iPS:420802 | HV-27 | CAGGTGCAGCTGGTGGAGTCTGGGGGAGGCGTGGTCCAGCCTGG | 390 |
| | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATGATGGACGCAATAAATACT | |
| | | ATGCACGCTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATGTTTTGACT | |
| | | GGTTACCCCGACTACTGGGGCCAGGGAACCCTGGTCACCGTCTCT | |
| | | AGT | |
| iPS:420809 | HV-28 | CAGGTGCAGCTGGTGGAGTCTGGGGGAGGCGTGGTCCAGCCTGG | 391 |
| | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATGATGGAAACAATAAATACT | |
| | | ATGCACGCTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATGTTTTGACT | |
| | | GGTTACCCCGACTACTGGGGCCAGGGAACCCTGGTCACCGTCTCT | |
| | | AGT | |
| iPS:420816 | HV-29 | CAGGTGCAGCTGGTGGAGTCTGGGGGAGGCGTGGTCCAGCCTGG | 392 |
| | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATGATGGACGCAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATATCTTGACT | |
| | | GGTTACCCCGACTACTGGGGCCAGGGAACCCTGGTCACCGTCTCT | |
| | | AGT | |
| iPS:420823 | HV-30 | CAGGTGCAGCTGGTGGAGTCTGGGGGAGGCGTGGTCCAGCCTGG | 393 |
| | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATGATGGAAACAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATATCTTGACT | |
| | | GGTTACCCCGACTACTGGGGCCAGGGAACCCTGGTCACCGTCTCT | |
| | | AGT | |
| iPS:420830 | HV-31 | CAGGTGCAGCTGGTGGAGTCTGGGGGAGGCGTGGTCCAGCCTGG | 394 |
| | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATGATGGAGGAAATAAATACT | |
| | | ATGCACGCTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATATCTTGACT | |
| | | GGTTACCCCGACTACTGGGGCCAGGGAACCCTGGTCACCGTCTCT | |
| | | AGT | |
| iPS:420901; | HV-32 | CAGGTGCAGCTGGTGGAGTCTGGGGGAGGCGTGGTCCAGCCTGG | 395 |
| iPS:421027 | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATAACGGACGCAATAAATACT | |
| | | ATGCACGCTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATGTTTTGACT | |
| | | GGTTACCCCGACTACTGGGGCCAGGGAACCCTGGTCACCGTCTCT | |
| | | AGT | |
| iPS:420908; | HV-33 | CAGGTGCAGCTGGTGGAGTCTGGGGGAGGCGTGGTCCAGCCTGG | 396 |
| iPS:421031 | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATATCGGACGCAATAAATACTA | |
| | | TGCACGCTCCGTGAAGGGCCGGTTCACCATCTCCAGAGACAATTC | |
| | | CAAGAACACCCTGTATCTGCAAATGAACAGCCTGAGAGCTGAGG | |
| | | ACACGGCTCTGTTTTACTGTGCGAGAGGATACGATGTTTTGACTG | |
| | | GTTACCCCGACTACTGGGGCCAGGGAACCCTGGTCACCGTCTCTA | |
| | | GT | |
| iPS:420915; | HV-34 | CAGGTGCAGCTGGTGGAGTCTGGGGGAGGCGTGGTCCAGCCTGG | 397 |
| iPS:421035 | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATCAGGGACGCAATAAATACT | |
| | | ATGCACGCTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATGTTTTGACT | |
| | | GGTTACCCCGACTACTGGGGCCAGGGAACCCTGGTCACCGTCTCT | |
| | | AGT | |
| iPS:420922; | HV-35 | CAGGTGCAGCTGGTGGAGTCTGGGGGAGGCGTGGTCCAGCCTGG | 398 |
| iPS:421039 | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATTACGGACGCAATAAATACTA | |
| | | TGCACGCTCCGTGAAGGGCCGGTTCACCATCTCCAGAGACAATTC | |
| | | CAAGAACACCCTGTATCTGCAAATGAACAGCCTGAGAGCTGAGG | |
| | | ACACGGCTCTGTTTTACTGTGCGAGAGGATACGATGTTTTGACTG | |
| | | GTTACCCCGACTACTGGGGCCAGGGAACCCTGGTCACCGTCTCTA | |
| | | GT | |
| iPS:420929; | HV-36 | CAGGTGCAGCTGGTGGAGTCTGGGGGAGGCGTGGTCCAGCCTGG | 399 |
| iPS:421043 | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATAACGGAAACAATAAATACT | |
| | | ATGCACGCTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATGTTTTGACT | |
| | | GGTTACCCCGACTACTGGGGCCAGGGAACCCTGGTCACCGTCTCT | |
| | | AGT | |
| iPS:420936; | HV-37 | CAGGTGCAGCTGGTGGAGTCTGGGGGAGGCGTGGTCCAGCCTGG | 400 |
| iPS:421047 | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATATCGGAAACAATAAATACT | |
| | | ATGCACGCTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATGTTTTGACT | |
| | | GGTTACCCCGACTACTGGGGCCAGGGAACCCTGGTCACCGTCTCT | |
| | | AGT | |
| iPS:420943; | HV-38 | CAGGTGCAGCTGGTGGAGTCTGGGGGAGGCGTGGTCCAGCCTGG | 401 |
| iPS:421051 | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATCAGGGAAACAATAAATACT | |
| | | ATGCACGCTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATGTTTTGACT | |
| | | GGTTACCCCGACTACTGGGGCCAGGGAACCCTGGTCACCGTCTCT | |
| | | AGT | |
| iPS:420950; | HV-39 | CAGGTGCAGCTGGTGGAGTCTGGGGGAGGCGTGGTCCAGCCTGG | 402 |
| iPS:421055 | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATTACGGAAACAATAAATACT | |
| | | ATGCACGCTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATGTTTTGACT | |
| | | GGTTACCCCGACTACTGGGGCCAGGGAACCCTGGTCACCGTCTCT | |
| | | AGT | |
| iPS:420957; | HV-40 | CAGGTGCAGCTGGTGGAGTCTGGGGGAGGCGTGGTCCAGCCTGG | 403 |
| iPS:421059 | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATAACGGACGCAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATATCTTGACT | |
| | | GGTTACCCCGACTACTGGGGCCAGGGAACCCTGGTCACCGTCTCT | |
| | | AGT | |
| iPS:420964; | HV-41 | CAGGTGCAGCTGGTGGAGTCTGGGGGAGGCGTGGTCCAGCCTGG | 404 |
| iPS:421063 | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATATCGGACGCAATAAATACTA | |
| | | TGCAGAGTCCGTGAAGGGCCGGTTCACCATCTCCAGAGACAATTC | |
| | | CAAGAACACCCTGTATCTGCAAATGAACAGCCTGAGAGCTGAGG | |
| | | ACACGGCTCTGTTTTACTGTGCGAGAGGATACGATATCTTGACTG | |
| | | GTTACCCCGACTACTGGGGCCAGGGAACCCTGGTCACCGTCTCTA | |
| | | GT | |
| iPS:420971; | HV-42 | CAGGTGCAGCTGGTGGAGTCTGGGGGAGGCGTGGTCCAGCCTGG | 405 |
| iPS:421067 | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATCAGGGACGCAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATATCTTGACT | |
| | | GGTTACCCCGACTACTGGGGCCAGGGAACCCTGGTCACCGTCTCT | |
| | | AGT | |
| iPS:420978; | HV-43 | CAGGTGCAGCTGGTGGAGTCTGGGGGAGGCGTGGTCCAGCCTGG | 406 |
| iPS:421071 | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATTACGGACGCAATAAATACTA | |
| | | TGCAGAGTCCGTGAAGGGCCGGTTCACCATCTCCAGAGACAATTC | |
| | | CAAGAACACCCTGTATCTGCAAATGAACAGCCTGAGAGCTGAGG | |
| | | ACACGGCTCTGTTTTACTGTGCGAGAGGATACGATATCTTGACTG | |
| | | GTTACCCCGACTACTGGGGCCAGGGAACCCTGGTCACCGTCTCTA | |
| | | GT | |
| iPS:420985; | HV-44 | CAGGTGCAGCTGGTGGAGTCTGGGGGAGGCGTGGTCCAGCCTGG | 407 |
| iPS:421075 | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATAACGGAAACAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATATCTTGACT | |
| | | GGTTACCCCGACTACTGGGGCCAGGGAACCCTGGTCACCGTCTCT | |
| | | AGT | |
| iPS:420992; | HV-45 | CAGGTGCAGCTGGTGGAGTCTGGGGGAGGCGTGGTCCAGCCTGG | 408 |
| iPS:421079 | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATATCGGAAACAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATATCTTGACT | |
| | | GGTTACCCCGACTACTGGGGCCAGGGAACCCTGGTCACCGTCTCT | |
| | | AGT | |
| iPS:420999; | HV-46 | CAGGTGCAGCTGGTGGAGTCTGGGGGAGGCGTGGTCCAGCCTGG | 409 |
| iPS:421083 | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATCAGGGAAACAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATATCTTGACT | |
| | | GGTTACCCCGACTACTGGGGCCAGGGAACCCTGGTCACCGTCTCT | |
| | | AGT | |
| iPS:421006; | HV-47 | CAGGTGCAGCTGGTGGAGTCTGGGGGAGGCGTGGTCCAGCCTGG | 410 |
| iPS:421087 | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATTACGGAAACAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATATCTTGACT | |
| | | GGTTACCCCGACTACTGGGGCCAGGGAACCCTGGTCACCGTCTCT | |
| | | AGT | |
| iPS:421013 | HV-48 | CAGGTGCAGCTGGTGGAGTCTGGGGGAGGCGTGGTCCAGCCTGG | 411 |
| | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATGATGGACGCAATAAATACT | |
| | | ATGCACGCTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATATCTTGACT | |
| | | GGTTACCCCGACTACTGGGGCCAGGGAACCCTGGTCACCGTCTCT | |
| | | AGT | |
| iPS:421020 | HV-49 | CAGGTGCAGCTGGTGGAGTCTGGGGGAGGCGTGGTCCAGCCTGG | 412 |
| | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATGATGGAAACAATAAATACT | |
| | | ATGCACGCTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATATCTTGACT | |
| | | GGTTACCCCGACTACTGGGGCCAGGGAACCCTGGTCACCGTCTCT | |
| | | AGT | |
| iPS:421091 | HV-50 | CAGGTGCAGCTGGTGGAGTCTGGGGGAGGCGTGGTCCAGCCTGG | 413 |
| | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATAACGGACGCAATAAATACT | |
| | | ATGCACGCTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATATCTTGACT | |
| | | GGTTACCCCGACTACTGGGGCCAGGGAACCCTGGTCACCGTCTCT | |
| | | AGT | |
| iPS:421098 | HV-51 | CAGGTGCAGCTGGTGGAGTCTGGGGGAGGCGTGGTCCAGCCTGG | 414 |
| | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATATCGGACGCAATAAATACTA | |
| | | TGCACGCTCCGTGAAGGGCCGGTTCACCATCTCCAGAGACAATTC | |
| | | CAAGAACACCCTGTATCTGCAAATGAACAGCCTGAGAGCTGAGG | |
| | | ACACGGCTCTGTTTTACTGTGCGAGAGGATACGATATCTTGACTG | |
| | | GTTACCCCGACTACTGGGGCCAGGGAACCCTGGTCACCGTCTCTA | |
| | | GT | |
| iPS:421105 | HV-52 | CAGGTGCAGCTGGTGGAGTCTGGGGGAGGCGTGGTCCAGCCTGG | 415 |
| | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATCAGGGACGCAATAAATACT | |
| | | ATGCACGCTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATATCTTGACT | |
| | | GGTTACCCCGACTACTGGGGCCAGGGAACCCTGGTCACCGTCTCT | |
| | | AGT | |
| iPS:421112 | HV-53 | CAGGTGCAGCTGGTGGAGTCTGGGGGAGGCGTGGTCCAGCCTGG | 416 |
| | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATTACGGACGCAATAAATACTA | |
| | | TGCACGCTCCGTGAAGGGCCGGTTCACCATCTCCAGAGACAATTC | |
| | | CAAGAACACCCTGTATCTGCAAATGAACAGCCTGAGAGCTGAGG | |
| | | ACACGGCTCTGTTTTACTGTGCGAGAGGATACGATATCTTGACTG | |
| | | GTTACCCCGACTACTGGGGCCAGGGAACCCTGGTCACCGTCTCTA | |
| | | GT | |
| iPS:421119 | HV-54 | CAGGTGCAGCTGGTGGAGTCTGGGGGAGGCGTGGTCCAGCCTGG | 417 |
| | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATAACGGAAACAATAAATACT | |
| | | ATGCACGCTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATATCTTGACT | |
| | | GGTTACCCCGACTACTGGGGCCAGGGAACCCTGGTCACCGTCTCT | |
| | | AGT | |
| iPS:421126 | HV-55 | CAGGTGCAGCTGGTGGAGTCTGGGGGAGGCGTGGTCCAGCCTGG | 418 |
| | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATATCGGAAACAATAAATACT | |
| | | ATGCACGCTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATATCTTGACT | |
| | | GGTTACCCCGACTACTGGGGCCAGGGAACCCTGGTCACCGTCTCT | |
| | | AGT | |
| iPS:421133 | HV-56 | CAGGTGCAGCTGGTGGAGTCTGGGGGAGGCGTGGTCCAGCCTGG | 419 |
| | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATCAGGGAAACAATAAATACT | |
| | | ATGCACGCTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATATCTTGACT | |
| | | GGTTACCCCGACTACTGGGGCCAGGGAACCCTGGTCACCGTCTCT | |
| | | AGT | |
| iPS:421140; | HV-57 | CAGGTGCAGCTGGTGGAGTCTGGGGGAGGCGTGGTCCAGCCTGG | 420 |
| iPS:421147 | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATTACGGAAACAATAAATACT | |
| | | ATGCACGCTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATATCTTGACT | |
| | | GGTTACCCCGACTACTGGGGCCAGGGAACCCTGGTCACCGTCTCT | |
| | | AGT | |
| iPS:421163; | HV-58 | CAGGTGCAGCTGGTGGAGTCTGGGGGAGGCGTGGTCCAGCCTGG | 421 |
| iPS:421207 | | GAGGTCCCTGCGACTCTCCTGTGCAGCCTCTGGATTCACCTTCAG | |
| | | TCACTTTGCCATGCACTGGGTCCGCCAGGCTCCAGGCAAGGGGCT | |
| | | GGAGTGGGTGGCAGTTATATCATATGATGGAGGAAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATGTTTTGACT | |
| | | GGTTACCCCGACTACTGGGGCCAGGGAACCCTGGTCACCGTCTCT | |
| | | AGT | |
| iPS:391578; | HV-59 | CAGGTGCAGCTGGTGGAGTCTGGGGGAGGCGTGGTCCAGCCTGG | 422 |
| iPS:421211 | | GAGGTCCCTGCGACTCTCCTGTGCAGCCTCTGGATTCACCTTCAG | |
| | | TAGATACGCCATGCACTGGGTCCGCCAGGCTCCAGGCAAGGGGC | |
| | | TGGAGTGGGTGGCAGTTATATCATATGATGGAGGAAATAAATAC | |
| | | TATGCAGAGTCCGTGAAGGGCCGGTTCACCATCTCCAGAGACAAT | |
| | | TCCAAGAACACCCTGTATCTGCAAATGAACAGCCTGAGAGCTGA | |
| | | GGACACGGCTCTGTTTTACTGTGCGAGAGGATACGATGTTTTGAC | |
| | | TGGTTACCCCGACTACTGGGGCCAGGGAACCCTGGTCACCGTCTC | |
| | | TAGT | |
| iPS:421170; | HV-60 | CAGGTGCAGCTGGTGGAGTCTGGGGGAGGCGTGGTCCAGCCTGG | 423 |
| iPS:421215 | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATTCGATGGAGGAAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATGTTTTGACT | |
| | | GGTTACCCCGACTACTGGGGCCAGGGAACCCTGGTCACCGTCTCT | |
| | | AGT | |
| iPS:421176; | HV-61 | CAGGTGCAGCTGGTGGAGTCTGGGGGAGGCGTGGTCCAGCCTGG | 424 |
| iPS:421219 | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATGATGGAGCCAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATGTTTTGACT | |
| | | GGTTACCCCGACTACTGGGGCCAGGGAACCCTGGTCACCGTCTCT | |
| | | AGT | |
| iPS:421182; | HV-62 | CAGGTGCAGCTGGTGGAGTCTGGGGGAGGCGTGGTCCAGCCTGG | 425 |
| iPS:421223 | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATGATGGAGGAAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATTTCTTGACT | |
| | | GGTTACCCCGACTACTGGGGCCAGGGAACCCTGGTCACCGTCTCT | |
| | | AGT | |
| iPS:421239 | HV-63 | CAGGTGCAGCTGGTGGAGTCTGGGGGAGGCGTGGTCCAGCCTGG | 426 |
| | | GAGGTCCCTGCGACTCTCCTGTGCAGCCTCTGGATTCACCTTCAG | |
| | | TCACTTTGCCATGCACTGGGTCCGCCAGGCTCCAGGCAAGGGGCT | |
| | | GGAGTGGGTGGCAGTTATATCATATAACGGAGGAAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATGTTTTGACT | |
| | | GGTTACCCCGACTACTGGGGCCAGGGAACCCTGGTCACCGTCTCT | |
| | | AGT | |
| iPS:421246 | HV-64 | CAGGTGCAGCTGGTGGAGTCTGGGGGAGGCGTGGTCCAGCCTGG | 427 |
| | | GAGGTCCCTGCGACTCTCCTGTGCAGCCTCTGGATTCACCTTCAG | |
| | | TAGATACGCCATGCACTGGGTCCGCCAGGCTCCAGGCAAGGGGC | |
| | | TGGAGTGGGTGGCAGTTATATCATATAACGGAGGAAATAAATAC | |
| | | TATGCAGAGTCCGTGAAGGGCCGGTTCACCATCTCCAGAGACAAT | |
| | | TCCAAGAACACCCTGTATCTGCAAATGAACAGCCTGAGAGCTGA | |
| | | GGACACGGCTCTGTTTTACTGTGCGAGAGGATACGATGTTTTGAC | |
| | | TGGTTACCCCGACTACTGGGGCCAGGGAACCCTGGTCACCGTCTC | |
| | | TAGT | |
| iPS:421253 | HV-65 | CAGGTGCAGCTGGTGGAGTCTGGGGGAGGCGTGGTCCAGCCTGG | 428 |
| | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATTCAACGGAGGAAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATGTTTTGACT | |
| | | GGTTACCCCGACTACTGGGGCCAGGGAACCCTGGTCACCGTCTCT | |
| | | AGT | |
| iPS:421260 | HV-66 | CAGGTGCAGCTGGTGGAGTCTGGGGGAGGCGTGGTCCAGCCTGG | 429 |
| | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATAACGGAGCCAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATGTTTTGACT | |
| | | GGTTACCCCGACTACTGGGGCCAGGGAACCCTGGTCACCGTCTCT | |
| | | AGT | |
| iPS:421267 | HV-67 | CAGGTGCAGCTGGTGGAGTCTGGGGGAGGCGTGGTCCAGCCTGG | 430 |
| | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATAACGGAGGAAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATTTCTTGACT | |
| | | GGTTACCCCGACTACTGGGGCCAGGGAACCCTGGTCACCGTCTCT | |
| | | AGT | |
| iPS:421286 | HV-68 | CAGGTGCAGCTGGTGGAGTCTGGGGGAGGCGTGGTCCAGCCTGG | 431 |
| | | GAGGTCCCTGCGACTCTCCTGTGCAGCCTCTGGATTCACCTTCAG | |
| | | TCACTTTGCCATGCACTGGGTCCGCCAGGCTCCAGGCAAGGGGCT | |
| | | GGAGTGGGTGGCAGTTATATCATATGATGGACGCAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATGTTTTGACT | |
| | | GGTTACCCCGACTACTGGGGCCAGGGAACCCTGGTCACCGTCTCT | |
| | | AGT | |
| iPS:421293 | HV-69 | CAGGTGCAGCTGGTGGAGTCTGGGGGAGGCGTGGTCCAGCCTGG | 432 |
| | | GAGGTCCCTGCGACTCTCCTGTGCAGCCTCTGGATTCACCTTCAG | |
| | | TAGATACGCCATGCACTGGGTCCGCCAGGCTCCAGGCAAGGGGC | |
| | | TGGAGTGGGTGGCAGTTATATCATATGATGGACGCAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATGTTTTGACT | |
| | | GGTTACCCCGACTACTGGGGCCAGGGAACCCTGGTCACCGTCTCT | |
| | | AGT | |
| iPS:421300 | HV-70 | CAGGTGCAGCTGGTGGAGTCTGGGGGAGGCGTGGTCCAGCCTGG | 433 |
| | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATTCGATGGACGCAATAAATACTA | |
| | | TGCAGAGTCCGTGAAGGGCCGGTTCACCATCTCCAGAGACAATTC | |
| | | CAAGAACACCCTGTATCTGCAAATGAACAGCCTGAGAGCTGAGG | |
| | | ACACGGCTCTGTTTTACTGTGCGAGAGGATACGATGTTTTGACTG | |
| | | GTTACCCCGACTACTGGGGCCAGGGAACCCTGGTCACCGTCTCTA | |
| | | GT | |
| iPS:421307 | HV-71 | CAGGTGCAGCTGGTGGAGTCTGGGGGAGGCGTGGTCCAGCCTGG | 434 |
| | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATGATGGACGCAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATTTCTTGACT | |
| | | GGTTACCCCGACTACTGGGGCCAGGGAACCCTGGTCACCGTCTCT | |
| | | AGT | |
| iPS:421326 | HV-72 | CAGGTGCAGCTGGTGGAGTCTGGGGGAGGCGTGGTCCAGCCTGG | 435 |
| | | GAGGTCCCTGCGACTCTCCTGTGCAGCCTCTGGATTCACCTTCAG | |
| | | TCACTTTGCCATGCACTGGGTCCGCCAGGCTCCAGGCAAGGGGCT | |
| | | GGAGTGGGTGGCAGTTATATCATATGATGGAGGAAATAAATACT | |
| | | ATGCACGCTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATGTTTTGACT | |
| | | GGTTACCCCGACTACTGGGGCCAGGGAACCCTGGTCACCGTCTCT | |
| | | AGT | |
| iPS:421333 | HV-73 | CAGGTGCAGCTGGTGGAGTCTGGGGGAGGCGTGGTCCAGCCTGG | 436 |
| | | GAGGTCCCTGCGACTCTCCTGTGCAGCCTCTGGATTCACCTTCAG | |
| | | TAGATACGCCATGCACTGGGTCCGCCAGGCTCCAGGCAAGGGGC | |
| | | TGGAGTGGGTGGCAGTTATATCATATGATGGAGGAAATAAATAC | |
| | | TATGCACGCTCCGTGAAGGGCCGGTTCACCATCTCCAGAGACAAT | |
| | | TCCAAGAACACCCTGTATCTGCAAATGAACAGCCTGAGAGCTGA | |
| | | GGACACGGCTCTGTTTTACTGTGCGAGAGGATACGATGTTTTGAC | |
| | | TGGTTACCCCGACTACTGGGGCCAGGGAACCCTGGTCACCGTCTC | |
| | | TAGT | |
| iPS:421340 | HV-74 | CAGGTGCAGCTGGTGGAGTCTGGGGGAGGCGTGGTCCAGCCTGG | 437 |
| | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATTCGATGGAGGAAATAAATACT | |
| | | ATGCACGCTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATGTTTTGACT | |
| | | GGTTACCCCGACTACTGGGGCCAGGGAACCCTGGTCACCGTCTCT | |
| | | AGT | |
| iPS:421347 | HV-75 | CAGGTGCAGCTGGTGGAGTCTGGGGGAGGCGTGGTCCAGCCTGG | 438 |
| | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATGATGGAGCCAATAAATACT | |
| | | ATGCACGCTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATGTTTTGACT | |
| | | GGTTACCCCGACTACTGGGGCCAGGGAACCCTGGTCACCGTCTCT | |
| | | AGT | |
| iPS:421354 | HV-76 | CAGGTGCAGCTGGTGGAGTCTGGGGGAGGCGTGGTCCAGCCTGG | 439 |
| | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATGATGGAGGAAATAAATACT | |
| | | ATGCACGCTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATTTCTTGACT | |
| | | GGTTACCCCGACTACTGGGGCCAGGGAACCCTGGTCACCGTCTCT | |
| | | AGT | |
| iPS:421373 | HV-77 | CAGGTGCAGCTGGTGGAGTCTGGGGGAGGCGTGGTCCAGCCTGG | 440 |
| | | GAGGTCCCTGCGACTCTCCTGTGCAGCCTCTGGATTCACCTTCAG | |
| | | TCACTTTGCCATGCACTGGGTCCGCCAGGCTCCAGGCAAGGGGCT | |
| | | GGAGTGGGTGGCAGTTATATCATATGATGGAGGAAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATATCTTGACT | |
| | | GGTTACCCCGACTACTGGGGCCAGGGAACCCTGGTCACCGTCTCT | |
| | | AGT | |
| iPS:421380 | HV-78 | CAGGTGCAGCTGGTGGAGTCTGGGGGAGGCGTGGTCCAGCCTGG | 441 |
| | | GAGGTCCCTGCGACTCTCCTGTGCAGCCTCTGGATTCACCTTCAG | |
| | | TAGATACGCCATGCACTGGGTCCGCCAGGCTCCAGGCAAGGGGC | |
| | | TGGAGTGGGTGGCAGTTATATCATATGATGGAGGAAATAAATAC | |
| | | TATGCAGAGTCCGTGAAGGGCCGGTTCACCATCTCCAGAGACAAT | |
| | | TCCAAGAACACCCTGTATCTGCAAATGAACAGCCTGAGAGCTGA | |
| | | GGACACGGCTCTGTTTTACTGTGCGAGAGGATACGATATCTTGAC | |
| | | TGGTTACCCCGACTACTGGGGCCAGGGAACCCTGGTCACCGTCTC | |
| | | TAGT | |
| iPS:421387 | HV-79 | CAGGTGCAGCTGGTGGAGTCTGGGGGAGGCGTGGTCCAGCCTGG | 442 |
| | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATTCGATGGAGGAAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATATCTTGACT | |
| | | GGTTACCCCGACTACTGGGGCCAGGGAACCCTGGTCACCGTCTCT | |
| | | AGT | |
| iPS:421394 | HV-80 | CAGGTGCAGCTGGTGGAGTCTGGGGGAGGCGTGGTCCAGCCTGG | 443 |
| | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCAGTTATATCATATGATGGAGCCAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATATCTTGACT | |
| | | GGTTACCCCGACTACTGGGGCCAGGGAACCCTGGTCACCGTCTCT | |
| | | AGT | |
| iPS:421855 | HV-81 | CAGGTGCAGCTGGTGGAGTCTGGGGGAGGCGTGGTCCAGCCTGG | 444 |
| | | GAGGTCCCTGCGACTCTCCTGTGCAGCCTCTGGATTCACCTTCAG | |
| | | TAAGTACGCCATGCACTGGGTCCGCCAGGCTCCAGGCAAGGGGC | |
| | | TGGAGTGGGTGGCAGTTATATCATTCAAGGGAAGCAATAAATAC | |
| | | TATGCAGAGTCCGTGAAGGGCCGGTTCACCATCTCCAGAGACAAT | |
| | | TCCAAGAACACCCTGTATCTGCAAATGAACAGCCTGAGAGCTGA | |
| | | GGACACGGCTCTGTTTTACTGTGCGAGAGGATACGATCTGTTGAC | |
| | | TGGTTACCCCGACTACTGGGGCCAGGGAACCCTGGTCACCGTCTC | |
| | | TAGT | |
| iPS:421861 | HV-82 | CAGGTGCAGCTGGTGGAGTCTGGGGGAGGCGTGGTCCAGCCTGG | 445 |
| | | GAGGTCCCTGCGACTCTCCTGTGCAGCCTCTGGATTCACCTTCAG | |
| | | TAGATACGCCATGCACTGGGTCCGCCAGGCTCCAGGCAAGGGGC | |
| | | TGGAGTGGGTGGCAGTTATATCATATCAGGGAGGAAATAAATAC | |
| | | TATGCAGAGTCCGTGAAGGGCCGGTTCACCATCTCCAGAGACAAT | |
| | | TCCAAGAACACCCTGTATCTGCAAATGAACAGCCTGAGAGCTGA | |
| | | GGACACGGCTCTGTTTTACTGTGCGAGAGGATACGATCTGTTGAC | |
| | | TGGTTACCCCGACTACTGGGGCCAGGGAACCCTGGTCACCGTCTC | |
| | | TAGT | |
| iPS:421867 | HV-83 | CAGGTGCAGCTGGTGGAGTCTGGGGGAGGCGTGGTCCAGCCTGG | 446 |
| | | GAGGTCCCTGCGACTCTCCTGTGCAGCCTCTGGATTCACCTTCAG | |
| | | TCACTTTGCCATGCACTGGGTCCGCCAGGCTCCAGGCAAGGGGCT | |
| | | GGAGTGGGTGGCAGTTATATCATTCAGCGGAAGCAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATATGTTGACT | |
| | | GGTTACCCCGACTACTGGGGCCAGGGAACCCTGGTCACCGTCTCT | |
| | | AGT | |
| iPS:421873 | HV-84 | CAGGTGCAGCTGGTGGAGTCTGGGGGAGGCGTGGTCCAGCCTGG | 447 |
| | | GAGGTCCCTGCGACTCTCCTGTGCAGCCTCTGGATTCACCTTCAG | |
| | | TAAGTTTGCCATGCACTGGGTCCGCCAGGCTCCAGGCAAGGGGCT | |
| | | GGAGTGGGTGGCAGTTATATCATATCGCGGAGGAAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATCTGTTGACT | |
| | | GGTTACCCCGACTACTGGGGCCAGGGAACCCTGGTCACCGTCTCT | |
| | | AGT | |
| iPS:421879 | HV-85 | CAGGTGCAGCTGGTGGAGTCTGGGGGAGGCGTGGTCCAGCCTGG | 448 |
| | | GAGGTCCCTGCGACTCTCCTGTGCAGCCTCTGGATTCACCTTCAG | |
| | | TAGATACGCCATGCACTGGGTCCGCCAGGCTCCAGGCAAGGGGC | |
| | | TGGAGTGGGTGGCAGTTATATCATATAGCGGAGCCAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATCTGTTGAGC | |
| | | GGTTACCCCGACTACTGGGGCCAGGGAACCCTGGTCACCGTCTCT | |
| | | AGT | |
| iPS:421885 | HV-86 | CAGGTGCAGCTGGTGGAGTCTGGGGGAGGCGTGGTCCAGCCTGG | 449 |
| | | GAGGTCCCTGCGACTCTCCTGTGCAGCCTCTGGATTCACCTTCAG | |
| | | TCACTACGCCATGCACTGGGTCCGCCAGGCTCCAGGCAAGGGGCT | |
| | | GGAGTGGGTGGCAGTTATATCATTCAAGGGAGCCAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATCTGTTGACT | |
| | | GGTTACCCCGACTACTGGGGCCAGGGAACCCTGGTCACCGTCTCT | |
| | | AGT | |
| iPS:421891 | HV-87 | CAGGTGCAGCTGGTGGAGTCTGGGGGAGGCGTGGTCCAGCCTGG | 450 |
| | | GAGGTCCCTGCGACTCTCCTGTGCAGCCTCTGGATTCACCTTCAG | |
| | | TCACTACGCCATGCACTGGGTCCGCCAGGCTCCAGGCAAGGGGCT | |
| | | GGAGTGGGTGGCAGTTATATCATATCGCGGAGCCAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATCTGTTGACT | |
| | | GGTTACCCCGACTACTGGGGCCAGGGAACCCTGGTCACCGTCTCT | |
| | | AGT | |
| iPS:421897 | HV-88 | CAGGTGCAGCTGGTGGAGTCTGGGGGAGGCGTGGTCCAGCCTGG | 451 |
| | | GAGGTCCCTGCGACTCTCCTGTGCAGCCTCTGGATTCACCTTCAG | |
| | | TCACTACGCCATGCACTGGGTCCGCCAGGCTCCAGGCAAGGGGCT | |
| | | GGAGTGGGTGGCAGTTATATCATTCTACGGAAGCAATAAATACTA | |
| | | TGCAGAGTCCGTGAAGGGCCGGTTCACCATCTCCAGAGACAATTC | |
| | | CAAGAACACCCTGTATCTGCAAATGAACAGCCTGAGAGCTGAGG | |
| | | ACACGGCTCTGTTTTACTGTGCGAGAGGATACGATTTCTTGACTG | |
| | | GTTACCCCGACTACTGGGGCCAGGGAACCCTGGTCACCGTCTCTA | |
| | | GT | |
| iPS:421903 | HV-89 | CAGGTGCAGCTGGTGGAGTCTGGGGGAGGCGTGGTCCAGCCTGG | 452 |
| | | GAGGTCCCTGCGACTCTCCTGTGCAGCCTCTGGATTCACCTTCAG | |
| | | TCACTTTGCCATGCACTGGGTCCGCCAGGCTCCAGGCAAGGGGCT | |
| | | GGAGTGGGTGGCAGTTATATCATTCTTCGGAGGAAATAAATACTA | |
| | | TGCAGAGTCCGTGAAGGGCCGGTTCACCATCTCCAGAGACAATTC | |
| | | CAAGAACACCCTGTATCTGCAAATGAACAGCCTGAGAGCTGAGG | |
| | | ACACGGCTCTGTTTTACTGTGCGAGAGGATACGATTTCTTGACTG | |
| | | GTTACCCCGACTACTGGGGCCAGGGAACCCTGGTCACCGTCTCTA | |
| | | GT | |
| iPS:421909 | HV-90 | CAGGTGCAGCTGGTGGAGTCTGGGGGAGGCGTGGTCCAGCCTGG | 453 |
| | | GAGGTCCCTGCGACTCTCCTGTGCAGCCTCTGGATTCACCTTCAG | |
| | | TCACTACGCCATGCACTGGGTCCGCCAGGCTCCAGGCAAGGGGCT | |
| | | GGAGTGGGTGGCAGTTATATCATTCATGGGAACCAATAAATACTA | |
| | | TGCAGAGTCCGTGAAGGGCCGGTTCACCATCTCCAGAGACAATTC | |
| | | CAAGAACACCCTGTATCTGCAAATGAACAGCCTGAGAGCTGAGG | |
| | | ACACGGCTCTGTTTTACTGTGCGAGAGGATACGATTTCTTGACTG | |
| | | GTTACCCCGACTACTGGGGCCAGGGAACCCTGGTCACCGTCTCTA | |
| | | GT | |
| iPS:421915 | HV-91 | CAGGTGCAGCTGGTGGAGTCTGGGGGAGGCGTGGTCCAGCCTGG | 454 |
| | | GAGGTCCCTGCGACTCTCCTGTGCAGCCTCTGGATTCACCTTCAG | |
| | | TTACTTTGCCATGCACTGGGTCCGCCAGGCTCCAGGCAAGGGGCT | |
| | | GGAGTGGGTGGCAGTTATATCACACCGCGGAACCAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATCTGTTGAGC | |
| | | GGTTACCCCGACTACTGGGGCCAGGGAACCCTGGTCACCGTCTCT | |
| | | AGT | |
| iPS:480711 | HV-92 | CAGGTGCAGCTGGTGGAGTCTGGGGGAGGCGTGGTCCAGCCTGG | 455 |
| | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGGTGTTATCAACTATCGTGGACATGGTAAATACTA | |
| | | TGCAG | |
| | | AGTCCGTGAAGGGCCGGTTCACCGTGTCCAGAGACAATTCCAAG | |
| | | AACACCCTGTATCTGCAAATGAACAGCCTGAGAGCTGAGGACAC | |
| | | GGCTCTGTTTTACTGTGCGAGAGGATACGATGTTTTGACTGGTTA | |
| | | CCCCGACTACTGGGGCCAGGGAACCCTGGTCACCGTGTCTAGT | |
| iPS:480706 | HV-93 | CAGGTGCAGCTGGTGGAGTCTGGGGGAGGCGTGGTCCAGCCTGG | 456 |
| | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGGTGTTATATCTTTTTCTGGAGGTTCTAAATACTA | |
| | | TGCAGAGTCCGTGAAGGGCCGGTTCACCTTGTCCAGAGACAATTC | |
| | | CAAGAACACCCTGTATCTGCAAATGAACAGCCTGAGAGCTGAGG | |
| | | ACACGGCTCTGTTTTACTGTGCGAGAGGATACGATGTTTTGACTG | |
| | | GTTACCCCGACTACTGGGGCCAGGGAACCCTGGTCACCGTGTCTA | |
| | | GT | |
| iPS:480713 | HV-94 | CAGGTGCAGCTGGTGGAGTCTGGGGGAGGCGTGGTCCAGCCTGG | 457 |
| | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGGTGTTATCTCTTATACTGGACAGTTCAAATACTA | |
| | | TGCAGAGTCCGTGAAGGGCCGGTTCACCGTGTCCAGAGACAATTC | |
| | | CAAGAACACCCTGTATCTGCAAATGAACAGCCTGAGAGCTGAGG | |
| | | ACACGGCTCTGTTTTACTGTGCGAGAGGATACGATGTTTTGACTG | |
| | | GTTACCCCGACTACTGGGGCCAGGGAACCCTGGTCACCGTGTCTA | |
| | | GT | |
| iPS:480705 | HV-95 | CAGGTGCAGCTGGTGGAGTCTGGGGGAGGCGTGGTCCAGCCTGG | 458 |
| | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGGTGTTATATCTTATACTGGAGCTCAGAAATACTA | |
| | | TGCAGAGTCCGTGAAGGGCCGGTTCACCATGTCCAGAGACAATTC | |
| | | CAAGAACACCCTGTATCTGCAAATGAACAGCCTGAGAGCTGAGG | |
| | | ACACGGCTCTGTTTTACTGTGCGAGAGGATACGATGTTTTGACTG | |
| | | GTTACCCCGACTACTGGGGCCAGGGAACCCTGGTCACCGTGTCTA | |
| | | GT | |
| iPS:480707 | HV-96 | CAGGTGCAGCTGGTGGAGTCTGGGGGAGGCGTGGTCCAGCCTGG | 459 |
| | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGGTGTTATATCTTATTCTGGAGCTTCTAAATACTA | |
| | | TGCAGAGTCCGTGAAGGGCCGGTTCACCATGTCCAGAGACAATTC | |
| | | CAAGAACACCCTGTATCTGCAAATGAACAGCCTGAGAGCTGAGG | |
| | | ACACGGCTCTGTTTTACTGTGCGAGAGGATACGATGTTTTGACTG | |
| | | GTTACCCCGACTACTGGGGCCAGGGAACCCTGGTCACCGTGTCTA | |
| | | GT | |
| iPS:480708 | HV-97 | CAGGTGCAGCTGGTGGAGTCTGGGGGAGGCGTGGTCCAGCCTGG | 460 |
| | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGCTGTTATATCTTATTCTGGAGCTTTCAAATACTA | |
| | | TGCAGAGTCCGTGAAGGGCCGGTTCACCGTGTCCAGAGACAATTC | |
| | | CAAGAACACCCTGTATCTGCAAATGAACAGCCTGAGAGCTGAGG | |
| | | ACACGGCTCTGTTTTACTGTGCGAGAGGATACGATGTTTTGACTG | |
| | | GTTACCCCGACTACTGGGGCCAGGGAACCCTGGTCACCGTGTCTA | |
| | | GT | |
| iPS:480709 | HV-98 | CAGGTGCAGCTGGTGGAGTCTGGGGGAGGCGTGGTCCAGCCTGG | 461 |
| | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGGTGTTATAACTTATACTGGAGGTGCTAAATACTA | |
| | | TGCAGAGTCCGTGAAGGGCCGGTTCACCATCTCCAGAGACAATTC | |
| | | CAAGAACACCCTGTATCTGCAAATGAACAGCCTGAGAGCTGAGG | |
| | | ACACGGCTCTGTTTTACTGTGCGAGAGGATACGATGTTTTGACTG | |
| | | GTTACCCCGACTACTGGGGCCAGGGAACCCTGGTCACCGTGTCTA | |
| | | GT | |
| iPS:480712 | HV-99 | CAGGTGCAGCTGGTGGAGTCTGGGGGAGGCGTGGTCCAGCCTGG | 462 |
| | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGGTGTTATCAACTTTCAGGGAACTACTAAATACTA | |
| | | TGCAGAGTCCGTGAAGGGCCGGTTCACCATCTCCAGAGACAATTC | |
| | | CAAGAACACCCTGTATCTGCAAATGAACAGCCTGAGAGCTGAGG | |
| | | ACACGGCTCTGTTTTACTGTGCGAGAGGATACGATGTTTTGACTG | |
| | | GTTACCCCGACTACTGGGGCCAGGGAACCCTGGTCACCGTGTCTA | |
| | | GT | |
| iPS:480704 | HV-100 | CAGGTGCAGCTGGTGGAGTCTGGGGGAGGCGTGGTCCAGCCTGG | 463 |
| | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGGTGTTATATCTTATTCTGGAGATCTGAAATACTA | |
| | | TGCAGAGTCCGTGAAGGGCCGGTTCACCGTGTCCAGAGACAATTC | |
| | | CAAGAACACCCTGTATCTGCAAATGAACAGCCTGAGAGCTGAGG | |
| | | ACACGGCTCTGTTTTACTGTGCGAGAGGATACGATGTTTTGACTG | |
| | | GTTACCCCGACTACTGGGGCCAGGGAACCCTGGTCACCGTGTCTA | |
| | | GT | |
| iPS:480710 | HV-101 | CAGGTGCAGCTGGTGGAGTCTGGGGGAGGCGTGGTCCAGCCTGG | 464 |
| | | GAGGTCCCTGCGACTCTCCTGTGCAGCCTCTGGATTCACCTTCAG | |
| | | TAGATTTGCCATGCACTGGGTCCGCCAGGCTCCAGGCAAGGGGCT | |
| | | GGAGTGGGTGGGTGTTATCAACTATTTCGGAGACGCTAAATACTA | |
| | | TGCAGAGTCCGTGAAGGGCCGGTTCACCATCTCCAGAGACAATTC | |
| | | CAAGAACACCCTGTATCTGCAAATGAACAGCCTGAGAGCTGAGG | |
| | | ACACGGCTCTGTTTTACTGTGCGAGAGGATACGATGTTTTGACTG | |
| | | GTTACCCCGACTACTGGGGCCAGGGAACCCTGGTCACCGTGTCTA | |
| | | GT | |
| iPS:480716 | HV-102 | CAGGTGCAGCTGGTGGAGTCTGGGGGAGGCGTGGTCCAGCCTGG | 465 |
| | | GAGGTCCCTGAGACTCTCCTGTGCAGCCTCTGGATTCACCTTCAG | |
| | | TTTCTACGCCATGCACTGGGTCCGCCAGGCTCCAGGCAAGGGGCT | |
| | | GGAGTGGGTGGCAGTTATCTCATCTTTCGGAAGTAATAAATACTA | |
| | | TGCAGAGTCCGTGAAGGGCCGGTTCACCATCTCCAGAGACAATTC | |
| | | CAAGAACACCCTGTATCTGCAAATGAACAGCCTGAGAGCTGAGG | |
| | | ACACGGCTCTGTTTTACTGTGCGAGAGGATACGATCTGCTGACTG | |
| | | GTTACCCCGACTACTGGGGCCAGGGAACCCTGGTCACCGTGTCTA | |
| | | GT | |
| iPS:480715 | HV-103 | CAGGTGCAGCTGGTGGAGTCTGGGGGAGGCGTGGTCCAGCCTGG | 466 |
| | | GAGGTCCCTGCGACTCTCCTGTGCAGCCTCTGGATTCACCTTCAG | |
| | | TTACTACGCCATGCACTGGGTCCGCCAGGCTCCAGGCAAGGGGCT | |
| | | GGAGTGGGTGGCAGTTATCTCATACTCTGGAAGTAATAAATACTA | |
| | | TGCAGAGTCCGTGAAGGGCCGGTTCACCATCTCCAGAGACAATTC | |
| | | CAAGAACACCCTGTATCTGCAAATGAACAGCCTGAGAGCTGAGG | |
| | | ACACGGCTCTGTTTTACTGTGCGAGAGGATACGATCTGCTGACTG | |
| | | GTTACCCCGACTACTGGGGCCAGGGAACCCTGGTCACCGTGTCTA | |
| | | GT | |
| iPS:480717 | HV-104 | CAGGTGCAGCTGGTGGAGTCTGGGGGAGGCGTGGTCCAGCCTGG | 467 |
| | | GAGGTCCCTGCGACTCTCCTGTGCAGCCTCTGGATTCACCTTCAG | |
| | | TTACTACGCCATGCACTGGGTCCGCCAGGCTCCAGGCAAGGGGCT | |
| | | GGAGTGGGTGGCAGTTATCTCACATTACGGAACTAATAAATACTA | |
| | | TGCAGAGTCCGTGAAGGGCCGGTTCACCATCTCCAGAGACAATTC | |
| | | CAAGAACACCCTGTATCTGCAAATGAACAGCCTGAGAGCTGAGG | |
| | | ACACGGCTCTGTTTTACTGTGCGAGAGGATACGATCCTCTGACTG | |
| | | GTTACCCCGACTACTGGGGCCAGGGAACCCTGGTCACCGTGTCTA | |
| | | GT | |
| iPS:480714 | HV-105 | CAGGTGCAGCTGGTGGAGTCTGGGGGAGGCGTGGTCCAGCCTGG | 468 |
| | | GAGGTCCCTGCGACTCTCCTGTGCAGCCTCTGGATTCACCTTCAG | |
| | | TCATTACGCCATGCACTGGGTCCGCCAGGCTCCAGGCAAGGGGCT | |
| | | GGAGTGGGTGGCAGTTATCTCATACCAGGGAAGTAATAAATACT | |
| | | ATGCAGAGTCCGTGAAGGGCCGGTTCACCATCTCCAGAGACAATT | |
| | | CCAAGAACACCCTGTATCTGCAAATGAACAGCCTGAGAGCTGAG | |
| | | GACACGGCTCTGTTTTACTGTGCGAGAGGATACGATCTGCTGACT | |
| | | GGTTACCCCGACTACTGGGGCCAGGGAACCCTGGTCACCGTGTCT | |
| | | AGT | |

19H8 variants

| | | | |
|---|---|---|---|
| 19H8; 3575 | HV-106 | CAGGTACAGCTGCAGCAGTCAGGTCCAGGACTGGTGAAGCCCTC | 469 |
| | | GCAGACCCTCTCACTCACCTGTGCCATCTCCGGGGACAGTGTCTC | |
| | | TAGCAACAGTGCTACTTGGAACTGGATCAGGCAGTCCCCATCGAG | |
| | | AGGCCTTGAGTGGCTGGGAAGGACATATTACAGGTCCAAGTGGT | |
| | | CTAATCATTATGCAGTATCTGTGAAAAGTCGAATAACCATCAACC | |
| | | CCGACACGTCCAAGAGCCAGTTCTCCCTGCAGCTGAACTCTGTGA | |
| | | CTCCCGAGGACACGGCTGTGTATTACTGTGCAAGAGGAACGTGG | |
| | | AAACAGCTATGGTTCCTTGACCACTGGGGCCAGGGAACCCTGGTC | |
| | | ACCGTCTCTAGTG | |
| iPS:448202; | HV-107 | CAGGTACAGCTGCAGCAGTCAGGTCCAGGACTGGTGAAGCCCTC | 470 |
| iPS:448689 | | GCAGACCCTCTCACTCACCTGTGCCATCTCCGGGGACAGTGTCTC | |
| | | TAACCGTCTGGCTACTTGGAACTGGATCAGGCAGTCCCCATCGAG | |
| | | AGGCCTTGAGTGGCTGGGAAGGACATACTACAGGGGTAAATGGA | |
| | | AAAATCATTATGCAGTATCTGTGAAAAGTCGAATAACCATCAACC | |
| | | CCGACACGTCCAAGAGCCAGTTCTCCCTGCAGCTGAACTCTGTGA | |
| | | CTCCCGAGGACACGGCTGTGTATTACTGTGCAAGAGGAACTTGGA | |
| | | ACCAGGACTGGTTCCTGGACCACTGGGGCCAGGGAACCCTGGTC | |
| | | ACCGTCTCTAGT | |
| iPS:449375 | HV-108 | CAGGTACAGCTGCAGCAGTCAGGTCCAGGACTGGTGAAGCCCTC | 471 |
| | | GCAGACCCTCTCACTCACCTGTGCCATCTCCGGGGACAGTGTCTC | |
| | | TAACCGTCTGGCTACTTGGAACTGGATCAGGCAGTCCCCATCGAG | |
| | | AGGCCTTGAGTGGCTGGGAAGGACATACTACAGGGGTAAATGGA | |
| | | AAAATCATTATGCAGTATCTGTGAAAAGTCGAATAACCATCAACC | |
| | | CCGACACGTCCAAGAGCCAGTTCTCCCTGCAGCTGAACTCTGTGA | |
| | | CTCCCGAGGACACGGCTGTGTATTACTGTGCAAGAGGAACTTGGG | |
| | | ACCAGGACTGGTTCCTGGACCACTGGGGCCAGGGAACCCTGGTC | |
| | | ACCGTCTCTAGT | |
| iPS:448083 | HV-109 | CAGGTACAGCTGCAGCAGTCAGGTCCAGGACTGGTGAAGCCCTC | 472 |
| | | GCAGACCCTCTCACTCACCTGTGCCATCTCCGGGGACAGTGTCTC | |
| | | TTCTCGTCAGGCTACTTGGAACTGGATCAGGCAGTCCCCATCGAG | |
| | | AGGCCTTGAGTGGCTGGGAAGGACATACTACAGGGGTAAATGGA | |
| | | AAAATCATTATGCAGTATCTGTGAAAAGTCGAATAACCATCAACC | |
| | | CCGACACGTCCAAGAGCCAGTTCTCCCTGCAGCTGAACTCTGTGA | |
| | | CTCCCGAGGACACGGCTGTGTATTACTGTGCAAGAGGAACTTGGG | |
| | | AACAGGACTGGTTCCTGGACCACTGGGGCCAGGGAACCCTGGTC | |
| | | ACCGTCTCTAGT | |
| iPS:452128; | HV-110 | CAGGTACAGCTGCAGCAGTCAGGTCCAGGACTGGTGAAGCCCTC | 473 |
| iPS:448195; | | GCAGACCCTCTCACTCACCTGTGCCATCTCCGGGGACAGTGTCTC | |
| iPS:448752; | | TAACAAACAGGCTACTTGGAACTGGATCAGGCAGTCCCCATCGA | |
| iPS:449027; | | GAGGCCTTGAGTGGCTGGGAAGGACATACTACAGGGGTAAATGG | |
| 3574 | | AAAAATCATTATGCAGTATCTGTGAAAAGTCGAATAACCATCAAC | |
| | | CCCGACACGTCCAAGAGCCAGTTCTCCCTGCAGCTGAACTCTGTG | |
| | | ACTCCCGAGGACACGGCTGTGTATTACTGTGCAAGAGGAATGTG | |
| | | GAACCAGAACTGGTTCCTGGACCACTGGGGCCAGGGAACCCTGG | |
| | | TCACCGTCTCTAGT | |
| iPS:448466 | HV-111 | CAGGTACAGCTGCAGCAGTCAGGTCCAGGACTGGTGAAGCCCTC | 474 |
| | | GCAGACCCTCTCACTCACCTGTGCCATCTCCGGGGACAGTGTCTC | |
| | | TAACAAACAGGCTACTTGGAACTGGATCAGGCAGTCCCCATCGA | |
| | | GAGGCCTTGAGTGGCTGGGAAGGACATACTACAGGGGTCAGTGG | |
| | | AAAAATCATTATGCAGTATCTGTGAAAAGTCGAATAACCATCAAC | |
| | | CCCGACACGTCCAAGAGCCAGTTCTCCCTGCAGCTGAACTCTGTG | |
| | | ACTCCCGAGGACACGGCTGTGTATTACTGTGCAAGAGGAACTTGG | |
| | | ATCGGTGACTGGTTCATGGACCACTGGGGCCAGGGAACCCTGGTC | |
| | | ACCGTCTCTAGT | |
| iPS:449034 | HV-112 | CAGGTACAGCTGCAGCAGTCAGGTCCAGGACTGGTGAAGCCCTC | 475 |
| | | GCAGACCCTCTCACTCACCTGTGCCATCTCCGGGGACAGTGTCTC | |
| | | TAACAAACAGGCTACTTGGAACTGGATCAGGCAGTCCCCATCGA | |
| | | GAGGCCTTGAGTGGCTGGGAAGGACATACTACAGGGGTAAATGG | |
| | | AAAAATCATTATGCAGTATCTGTGAAAAGTCGAATAACCATCAAC | |
| | | CCCGACACGTCCAAGAGCCAGTTCTCCCTGCAGCTGAACTCTGTG | |
| | | ACTCCCGAGGACACGGCTGTGTATTACTGTGCAAGAGGAACTTGG | |
| | | ATCCAGGACTGGTTCCTGGACCACTGGGGCCAGGGAACCCTGGTC | |
| | | ACCGTCTCTAGT | |
| iPS:448075 | HV-113 | CAGGTACAGCTGCAGCAGTCAGGTCCAGGACTGGTGAAGCCCTC | 476 |
| | | GCAGACCCTCTCACTCACCTGTGCCATCTCCGGGGACAGTGTCTC | |
| | | TTCTAACCATGCTACTTGGAACTGGATCAGGCAGTCCCCATCGAG | |
| | | AGGCCTTGAGTGGCTGGGAAGGACATACTACAGGGGTAAATGGA | |
| | | AAAATCATTATGCAGTATCTGTGAAAAGTCGAATAACCATCAACC | |
| | | CCGACACGTCCAAGAGCCAGTTCTCCCTGCAGCTGAACTCTGTGA | |
| | | CTCCCGAGGACACGGCTGTGTATTACTGTGCAAGAGGAACTTGGG | |
| | | ACCAGGACTGGTTCCTGGACCACTGGGGCCAGGGAACCCTGGTC | |
| | | ACCGTCTCTAGT | |
| iPS:448924 | HV-114 | CAGGTACAGCTGCAGCAGTCAGGTCCAGGACTGGTGAAGCCCTC | 477 |
| | | GCAGACCCTCTCACTCACCTGTGCCATCTCCGGGGACAGTGTCTC | |
| | | TTCTCGTTACGCTACTTGGAACTGGATCAGGCAGTCCCCATCGAG | |
| | | AGGCCTTGAGTGGCTGGGAAGGACATACTACAGGGGTCAGTGGA | |
| | | AAAATCATTATGCAGTATCTGTGAAAAGTCGAATAACCATCAACC | |
| | | CCGACACGTCCAAGAGCCAGTTCTCCCTGCAGCTGAACTCTGTGA | |
| | | CTCCCGAGGACACGGCTGTGTATTACTGTGCAAGAGGAATGTGG | |
| | | AACCAGAACTGGTTCCTGGACCACTGGGGCCAGGGAACCCTGGT | |
| | | CACCGTCTCTAGT | |
| iPS:448772 | HV-115 | CAGGTACAGCTGCAGCAGTCAGGTCCAGGACTGGTGAAGCCCTC | 478 |
| | | GCAGACCCTCTCACTCACCTGTGCCATCTCCGGGGACAGTGTCTC | |
| | | TAACAAACAGGCTACTTGGAACTGGATCAGGCAGTCCCCATCGA | |
| | | GAGGCCTTGAGTGGCTGGGAAGGACATACTACAGGGGTAAATGG | |
| | | AAAAATCATTATGCAGTATCTGTGAAAAGTCGAATAACCATCAAC | |
| | | CCCGACACGTCCAAGAGCCAGTTCTCCCTGCAGCTGAACTCTGTG | |
| | | ACTCCCGAGGACACGGCTGTGTATTACTGTGCAAGAGGAATGTG | |
| | | GTCTGGTGACTGGTTCCTGGACCACTGGGGCCAGGGAACCCTGGT | |
| | | CACCGTCTCTAGT | |
| iPS:448117 | HV-116 | CAGGTACAGCTGCAGCAGTCAGGTCCAGGACTGGTGAAGCCCTC | 479 |
| | | GCAGACCCTCTCACTCACCTGTGCCATCTCCGGGGACAGTGTCTC | |
| | | TTCTCATGTTGCTACTTGGAACTGGATCAGGCAGTCCCCATCGAG | |
| | | AGGCCTTGAGTGGCTGGGAAGGACATACTACAGGGGTAAATGGA | |
| | | AAAATCATTATGCAGTATCTGTGAAAAGTCGAATAACCATCAACC | |
| | | CCGACACGTCCAAGAGCCAGTTCTCCCTGCAGCTGAACTCTGTGA | |
| | | CTCCCGAGGACACGGCTGTGTATTACTGTGCAAGAGGAATGTGGT | |
| | | CTGAAGACTGGTTCCTGGACCACTGGGGCCAGGGAACCCTGGTC | |
| | | ACCGTCTCTAGT | |
| iPS:448788 | HV-117 | CAGGTACAGCTGCAGCAGTCAGGTCCAGGACTGGTGAAGCCCTC | 480 |
| | | GCAGACCCTCTCACTCACCTGTGCCATCTCCGGGGACAGTGTCTC | |
| | | TTCTCGTCAGGCTACTTGGAACTGGATCAGGCAGTCCCCATCGAG | |
| | | AGGCCTTGAGTGGCTGGGAAGGACATACTACAGGGGTAAATGGA | |
| | | AAAATCATTATGCAGTATCTGTGAAAAGTCGAATAACCATCAACC | |
| | | CCGACACGTCCAAGAGCCAGTTCTCCCTGCAGCTGAACTCTGTGA | |
| | | CTCCCGAGGACACGGCTGTGTATTACTGTGCAAGAGGAATGTGGC | |
| | | AGGGTAACTGGTTCCTGGACCACTGGGGCCAGGGAACCCTGGTC | |
| | | ACCGTCTCTAGT | |
| iPS:448593 | HV-118 | CAGGTACAGCTGCAGCAGTCAGGTCCAGGACTGGTGAAGCCCTC | 481 |
| | | GCAGACCCTCTCACTCACCTGTGCCATCTCCGGGGACAGTGTCTC | |
| | | TAACCATCAGGCTACTTGGAACTGGATCAGGCAGTCCCCATCGAG | |
| | | AGGCCTTGAGTGGCTGGGAAGGACATACTACAGGGGTAAATGGA | |
| | | AAAATCATTATGCAGTATCTGTGAAAAGTCGAATAACCATCAACC | |
| | | CCGACACGTCCAAGAGCCAGTTCTCCCTGCAGCTGAACTCTGTGA | |
| | | CTCCCGAGGACACGGCTGTGTATTACTGTGCAAGAGGAACTTGGA | |
| | | TCCAGGACTGGTTCCTGGACCACTGGGGCCAGGGAACCCTGGTCA | |
| | | CCGTCTCTAGT | |
| iPS:448238 | HV-119 | CAGGTACAGCTGCAGCAGTCAGGTCCAGGACTGGTGAAGCCCTC | 482 |
| | | GCAGACCCTCTCACTCACCTGTGCCATCTCCGGGGACAGTGTCTC | |
| | | TTCTCGTGACGCTACTTGGAACTGGATCAGGCAGTCCCCATCGAG | |
| | | AGGCCTTGAGTGGCTGGGAAGGACATACTACAGGGGTAAATGGA | |
| | | AAAATCATTATGCAGTATCTGTGAAAAGTCGAATAACCATCAACC | |
| | | CCGACACGTCCAAGAGCCAGTTCTCCCTGCAGCTGAACTCTGTGA | |
| | | CTCCCGAGGACACGGCTGTGTATTACTGTGCAAGAGGACAGTGG | |
| | | AACGAAGACTGGTTCCTGGACCACTGGGGCCAGGGAACCCTGGT | |
| | | CACCGTCTCTAGT | |
| iPS:448901 | HV-120 | CAGGTACAGCTGCAGCAGTCAGGTCCAGGACTGGTGAAGCCCTC | 483 |
| | | GCAGACCCTCTCACTCACCTGTGCCATCTCCGGGGACAGTGTCTC | |
| | | TAACCGTCTGGCTACTTGGAACTGGATCAGGCAGTCCCCATCGAG | |
| | | AGGCCTTGAGTGGCTGGGAAGGACATACTACAGGGGTAAATGGA | |
| | | AAAATCATTATGCAGTATCTGTGAAAAGTCGAATAACCATCAACC | |
| | | CCGACACGTCCAAGAGCCAGTTCTCCCTGCAGCTGAACTCTGTGA | |
| | | CTCCCGAGGACACGGCTGTGTATTACTGTGCAAGAGGACGTTGGG | |
| | | AAGGTGACTGGTTCTTCGACCACTGGGGCCAGGGAACCCTGGTCA | |
| | | CCGTCTCTAGT | |
| iPS:448655 | HV-121 | CAGGTACAGCTGCAGCAGTCAGGTCCAGGACTGGTGAAGCCCTC | 484 |
| | | GCAGACCCTCTCACTCACCTGTGCCATCTCCGGGGACAGTGTCTC | |
| | | TAACAAACAGGCTACTTGGAACTGGATCAGGCAGTCCCCATCGA | |
| | | GAGGCCTTGAGTGGCTGGGAAGGACATACTTCAGGCGTACTTGG | |
| | | AAAAATCATTATGCAGTATCTGTGAAAAGTCGAATAACCATCAAC | |
| | | CCCGACACGTCCAAGAGCCAGTTCTCCCTGCAGCTGAACTCTGTG | |
| | | ACTCCCGAGGACACGGCTGTGTATTACTGTGCAAGAGGAATGTG | |
| | | GTCTGAAGACTGGTTCCTGGACCACTGGGGCCAGGGAACCCTGGT | |
| | | CACCGTCTCTAGT | |
| iPS:448730 | HV-122 | CAGGTACAGCTGCAGCAGTCAGGTCCAGGACTGGTGAAGCCCTC | 485 |
| | | GCAGACCCTCTCACTCACCTGTGCCATCTCCGGGGACAGTGTCTC | |
| | | TAACCGTCTGGCTACTTGGAACTGGATCAGGCAGTCCCCATCGAG | |
| | | AGGCCTTGAGTGGCTGGGAAGGACATACTACAGGGGTAAATGGA | |
| | | AAAATCATTATGCAGTATCTGTGAAAAGTCGAATAACCATCAACC | |
| | | CCGACACGTCCAAGAGCCAGTTCTCCCTGCAGCTGAACTCTGTGA | |
| | | CTCCCGAGGACACGGCTGTGTATTACTGTGCAAGAGGAGTTTGGA | |
| | | TCGGTAACTGGTTCCTGGACCACTGGGGCCAGGGAACCCTGGTCA | |
| | | CCGTCTCTAGT | |

Isolated nucleic acids encoding the anti-PAC1 antibodies or antigen-binding fragments of the invention may comprise a nucleotide sequence that is at least 80% identical, at least 90% identical, at least 95% identical, or at least 98% identical to any of the nucleotide sequences listed in Tables 4A, 4B, 5A, and 5B. In some embodiments, an isolated nucleic acid encoding an anti-PAC1 antibody light chain variable region comprises a sequence that is at least 80% identical, at least 90% identical, at least 95% identical, or at least 98% identical to a sequence selected from SEQ ID NOs: 328 to 342. In other embodiments, an isolated nucleic acid encoding an anti-PAC1 antibody light chain variable region comprises a sequence that is at least 80% identical, at least 90% identical, at least 95% identical, or at least 98% identical to a sequence selected from SEQ ID NOs: 343 to 363. In certain embodiments, an isolated nucleic acid encoding an anti-PAC1 antibody light chain variable region comprises a sequence selected from SEQ ID NOs: 328 to 342. In certain other embodiments, an isolated nucleic acid encoding an anti-PAC1 antibody light chain variable region comprises a sequence selected from SEQ ID NOs: 343 to 363. In related embodiments, an isolated nucleic acid encoding an anti-PAC1 antibody heavy chain variable region comprises a sequence that is at least 80% identical, at least 90% identical, at least 95% identical, or at least 98% identical to a sequence selected from SEQ ID NOs: 364 to 468. In other related embodiments, an isolated nucleic acid encoding an anti-PAC1 antibody heavy chain variable region comprises a sequence that is at least 80% identical, at least 90% identical, at least 95% identical, or at least 98% identical to a sequence selected from SEQ ID NOs: 469 to 485. In some embodiments, an isolated nucleic acid encoding an anti-PAC1 antibody heavy chain variable region comprises a sequence selected from SEQ ID NOs: 364 to 468. In other embodiments, an isolated nucleic acid encoding an anti-PAC1 antibody heavy chain variable region comprises a sequence selected from SEQ ID NOs: 469 to 485.

In some embodiments, an isolated nucleic acid encoding an anti-PAC1 antibody light chain comprises a sequence that is at least 80% identical, at least 90% identical, at least 95% identical, or at least 98% identical to a sequence selected from SEQ ID NOs: 537 to 541. In other embodiments, an isolated nucleic acid encoding an anti-PAC1 antibody light chain comprises a sequence that is at least 80% identical, at least 90% identical, at least 95% identical, or at least 98% identical to a sequence selected from SEQ ID NOs: 542 to 551. In certain embodiments, an isolated nucleic acid encoding an anti-PAC1 antibody light chain comprises a sequence selected from SEQ ID NOs: 537 to 541. In certain other embodiments, an isolated nucleic acid encoding an anti-PAC1 antibody light chain comprises a sequence selected from SEQ ID NOs: 542 to 551. In related embodiments, an isolated nucleic acid encoding an anti-PAC1 antibody heavy chain comprises a sequence that is at least 80% identical, at least 90% identical, at least 95% identical, or at least 98% identical to a sequence selected from SEQ ID NOs: 552 to 562. In other related embodiments, an isolated nucleic acid encoding an anti-PAC1 antibody heavy chain comprises a sequence that is at least 80% identical, at least 90% identical, at least 95% identical, or at least 98% identical to a sequence selected from SEQ ID NOs: 563 to 569. In some embodiments, an isolated nucleic acid encoding an anti-PAC1 antibody heavy chain comprises a sequence selected from SEQ ID NOs: 552 to 562. In other embodiments, an isolated nucleic acid encoding an anti-PAC1 antibody heavy chain comprises a sequence selected from SEQ ID NOs: 563 to 569.

The nucleic acid sequences provided in Tables 4A, 4B, 5A, and 5B are exemplary only. As will be appreciated by those in the art, due to the degeneracy of the genetic code, an extremely large number of nucleic acids may be made, all of which encode the CDRs, variable regions, and heavy and light chains or other components of the antibodies and antigen-binding fragments described herein. Thus, having identified a particular amino acid sequence, those skilled in the art could make any number of different nucleic acids, by simply modifying the sequence of one or more codons in a way which does not change the amino acid sequence of the encoded protein.

The present invention also includes vectors comprising one or more nucleic acids encoding one or more components of the antibodies or antigen-binding fragments of the invention (e.g. variable regions, light chains, and heavy chains). The term “vector” refers to any molecule or entity (e.g., nucleic acid, plasmid, bacteriophage or virus) used to transfer protein coding information into a host cell. Examples of vectors include, but are not limited to, plasmids, viral vectors, non-episomal mammalian vectors and expression vectors, for example, recombinant expression vectors. The term “expression vector” or “expression construct” as used herein refers to a recombinant DNA molecule containing a desired coding sequence and appropriate nucleic acid control sequences necessary for the expression of the operably linked coding sequence in a particular host cell. An expression vector can include, but is not limited to, sequences that affect or control transcription, translation, and, if introns are present, affect RNA splicing of a coding region operably linked thereto. Nucleic acid sequences necessary for expression in prokaryotes include a promoter, optionally an operator sequence, a ribosome binding site and possibly other sequences. Eukaryotic cells are known to utilize promoters, enhancers, and termination and polyadenylation signals.

A secretory signal peptide sequence can also, optionally, be encoded by the expression vector, operably linked to the coding sequence of interest, so that the expressed polypeptide can be secreted by the recombinant host cell, for more facile isolation of the polypeptide of interest from the cell, if desired. For instance, in some embodiments, signal peptide sequences may be appended/fused to the amino terminus of any of the variable region polypeptide sequences listed in Tables 1A and 1B or any of the full chain polypeptide sequences listed in Tables 4A and 4B. In certain embodiments, a signal peptide having the amino acid sequence of MDMRVPAQLLGLLLLWLRGARC (SEQ ID NO: 486) is fused to the amino terminus of any of the variable region polypeptide sequences in Tables 1A and 1B or full chain polypeptide sequences in Tables 4A and 4B. In other embodiments, a signal peptide having the amino acid sequence of MAWALLLLTLLTQGTGSWA (SEQ ID NO: 487) is fused to the amino terminus of any of the variable region polypeptide sequences in Tables 1A and 1B or full chain polypeptide sequences in Tables 4A and 4B. In still other embodiments, a signal peptide having the amino acid sequence of MTCSPLLLTLLIHCTGSWA (SEQ ID NO: 488) is fused to the amino terminus of any of the variable region polypeptide sequences in Tables 1A and 1B or full chain polypeptide sequences in Tables 4A and 4B. Other suitable signal peptide sequences that can be fused to the amino terminus of the variable region polypeptide sequences or full chain polypeptide sequences described herein include: MEAPAQLLFLLLLWLPDTTG (SEQ ID NO: 489), MEWTWRVLFLVAAATGAHS (SEQ ID NO: 490), METPAQLLFLLLLWLPDTTG (SEQ ID NO: 491), METPAQLLFLLLLWLPDTTG (SEQ ID NO: 492), MKHLWFFLLLVAAPRWVLS (SEQ ID NO: 493), MEWSWVFLFFLSVTTGVHS (SEQ ID NO: 494), MDIRAPTQLLGLLLLWLPGAKC (SEQ ID NO: 495), MDIRAPTQLLGLLLLWLPGARC (SEQ ID NO: 496), MDTRAPTQLLGLLLLWLPGATF (SEQ ID NO: 497), MDTRAPTQLLGLLLLWLPGARC (SEQ ID NO: 498), METGLRWLLLVAVLKGVQC (SEQ ID NO: 499), METGLRWLLLVAVLKGVQCQE (SEQ ID NO: 500), and MDMRAPTQLLGLLLLWLPGARC (SEQ ID NO: 501). Other signal or secretory peptides are known to those of skill in the art and may be fused to any of the variable region polypeptide chains listed in Tables 1A and 1B or full chain polypeptide sequences in Tables 4A and 4B, for example, to facilitate or optimize expression in particular host cells.

Typically, expression vectors used in the host cells to produce the anti-PAC1 antibodies and antigen-binding fragments of the invention will contain sequences for plasmid maintenance and for cloning and expression of exogenous nucleotide sequences encoding the components of the antibodies and antigen-binding fragments. Such sequences, collectively referred to as “flanking sequences,” in certain embodiments will typically include one or more of the following nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Each of these sequences is discussed below.

Optionally, the vector may contain a “tag”-encoding sequence, i.e., an oligonucleotide molecule located at the 5′ or 3′ end of the polypeptide coding sequence; the oligonucleotide tag sequence encodes polyHis (such as hexaHis), FLAG, HA (hemaglutinin influenza virus), myc, or another “tag” molecule for which commercially available antibodies exist. This tag is typically fused to the polypeptide upon expression of the polypeptide, and can serve as a means for affinity purification or detection of the polypeptide from the host cell. Affinity purification can be accomplished, for example, by column chromatography using antibodies against the tag as an affinity matrix. Optionally, the tag can subsequently be removed from the purified polypeptide by various means such as using certain peptidases for cleavage.

Flanking sequences may be homologous (i.e., from the same species and/or strain as the host cell), heterologous (i.e., from a species other than the host cell species or strain), hybrid (i.e., a combination of flanking sequences from more than one source), synthetic or native. As such, the source of a flanking sequence may be any prokaryotic or eukaryotic organism, any vertebrate or invertebrate organism, or any plant, provided that the flanking sequence is functional in, and can be activated by, the host cell machinery.

Flanking sequences useful in the vectors of this invention may be obtained by any of several methods well known in the art. Typically, flanking sequences useful herein will have been previously identified by mapping and/or by restriction endonuclease digestion and can thus be isolated from the proper tissue source using the appropriate restriction endonucleases. In some cases, the full nucleotide sequence of a flanking sequence may be known. Here, the flanking sequence may be synthesized using routine methods for nucleic acid synthesis or cloning.

Whether all or only a portion of the flanking sequence is known, it may be obtained using polymerase chain reaction (PCR) and/or by screening a genomic library with a suitable probe such as an oligonucleotide and/or flanking sequence fragment from the same or another species. Where the flanking sequence is not known, a fragment of DNA containing a flanking sequence may be isolated from a larger piece of DNA that may contain, for example, a coding sequence or even another gene or genes. Isolation may be accomplished by restriction endonuclease digestion to produce the proper DNA fragment followed by isolation using agarose gel purification, Qiagen® column chromatography (Chatsworth, CA), or other methods known to the skilled artisan. The selection of suitable enzymes to accomplish this purpose will be readily apparent to one of ordinary skill in the art.

An origin of replication is typically a part of those prokaryotic expression vectors purchased commercially, and the origin aids in the amplification of the vector in a host cell. If the vector of choice does not contain an origin of replication site, one may be chemically synthesized based on a known sequence, and ligated into the vector. For example, the origin of replication from the plasmid pBR322 (New England Biolabs, Beverly, MA) is suitable for most gram-negative bacteria, and various viral origins (e.g., SV40, polyoma, adenovirus, vesicular stomatitus virus (VSV), or papillomaviruses such as HPV or BPV) are useful for cloning vectors in mammalian cells. Generally, the origin of replication component is not needed for mammalian expression vectors (for example, the SV40 origin is often used only because it also contains the virus early promoter).

A transcription termination sequence is typically located 3′ to the end of a polypeptide coding region and serves to terminate transcription. Usually, a transcription termination sequence in prokaryotic cells is a G-C rich fragment followed by a poly-T sequence. While the sequence is easily cloned from a library or even purchased commercially as part of a vector, it can also be readily synthesized using known methods for nucleic acid synthesis.

A selectable marker gene encodes a protein necessary for the survival and growth of a host cell grown in a selective culture medium. Typical selection marker genes encode proteins that (a) confer resistance to antibiotics or other toxins, e.g., ampicillin, tetracycline, or kanamycin for prokaryotic host cells; (b) complement auxotrophic deficiencies of the cell; or (c) supply critical nutrients not available from complex or defined media. Specific selectable markers are the kanamycin resistance gene, the ampicillin resistance gene, and the tetracycline resistance gene. Advantageously, a neomycin resistance gene may also be used for selection in both prokaryotic and eukaryotic host cells.

Other selectable genes may be used to amplify the gene that will be expressed. Amplification is the process wherein genes that are required for production of a protein critical for growth or cell survival are reiterated in tandem within the chromosomes of successive generations of recombinant cells. Examples of suitable selectable markers for mammalian cells include dihydrofolate reductase (DHFR) and promoterless thymidine kinase genes. Mammalian cell transformants are placed under selection pressure wherein only the transformants are uniquely adapted to survive by virtue of the selectable gene present in the vector. Selection pressure is imposed by culturing the transformed cells under conditions in which the concentration of selection agent in the medium is successively increased, thereby leading to the amplification of both the selectable gene and the DNA that encodes another gene, such as one or more components of the antibodies or antigen-binding fragments described herein. As a result, increased quantities of a polypeptide are synthesized from the amplified DNA.

A ribosome-binding site is usually necessary for translation initiation of mRNA and is characterized by a Shine-Dalgarno sequence (prokaryotes) or a Kozak sequence (eukaryotes). The element is typically located 3′ to the promoter and 5′ to the coding sequence of the polypeptide to be expressed. In certain embodiments, one or more coding regions may be operably linked to an internal ribosome binding site (IRES), allowing translation of two open reading frames from a single RNA transcript.

In some cases, such as where glycosylation is desired in a eukaryotic host cell expression system, one may manipulate the various pre- or prosequences to improve glycosylation or yield. For example, one may alter the peptidase cleavage site of a particular signal peptide, or add prosequences, which also may affect glycosylation. The final protein product may have, in the −1 position (relative to the first amino acid of the mature protein) one or more additional amino acids incident to expression, which may not have been totally removed. For example, the final protein product may have one or two amino acid residues found in the peptidase cleavage site, attached to the amino-terminus. Alternatively, use of some enzyme cleavage sites may result in a slightly truncated form of the desired polypeptide, if the enzyme cuts at such area within the mature polypeptide.

Expression and cloning vectors of the invention will typically contain a promoter that is recognized by the host organism and operably linked to the molecule encoding the polypeptide. The term “operably linked” as used herein refers to the linkage of two or more nucleic acid sequences in such a manner that a nucleic acid molecule capable of directing the transcription of a given gene and/or the synthesis of a desired protein molecule is produced. For example, a control sequence in a vector that is “operably linked” to a protein coding sequence is ligated thereto so that expression of the protein coding sequence is achieved under conditions compatible with the transcriptional activity of the control sequences. More specifically, a promoter and/or enhancer sequence, including any combination of cis-acting transcriptional control elements is operably linked to a coding sequence if it stimulates or modulates the transcription of the coding sequence in an appropriate host cell or other expression system.

Promoters are non-transcribed sequences located upstream (i.e., 5′) to the start codon of a structural gene (generally within about 100 to 1000 bp) that control transcription of the structural gene. Promoters are conventionally grouped into one of two classes: inducible promoters and constitutive promoters. Inducible promoters initiate increased levels of transcription from DNA under their control in response to some change in culture conditions, such as the presence or absence of a nutrient or a change in temperature. Constitutive promoters, on the other hand, uniformly transcribe a gene to which they are operably linked, that is, with little or no control over gene expression. A large number of promoters, recognized by a variety of potential host cells, are well known. A suitable promoter is operably linked to the DNA encoding e.g., heavy chain, light chain, or other component of the antibodies and antigen-binding fragments of the invention, by removing the promoter from the source DNA by restriction enzyme digestion and inserting the desired promoter sequence into the vector.

Suitable promoters for use with yeast hosts are also well known in the art. Yeast enhancers are advantageously used with yeast promoters. Suitable promoters for use with mammalian host cells are well known and include, but are not limited to, those obtained from the genomes of viruses such as polyoma virus, fowlpox virus, adenovirus (such as Adenovirus serotypes 2, 8, or 9), bovine papilloma virus, avian sarcoma virus, cytomegalovirus, retroviruses, hepatitis-B virus and Simian Virus 40 (SV40). Other suitable mammalian promoters include heterologous mammalian promoters, for example, heat-shock promoters and the actin promoter.

Additional specific promoters which may be of interest include, but are not limited to: SV40 early promoter (Benoist and Chambon, 1981, Nature 290:304-310); CMV promoter (Thornsen et al., 1984, Proc. Natl. Acad. U.S.A. 81:659-663); the promoter contained in the 3′ long terminal repeat of Rous sarcoma virus (Yamamoto et al., 1980, Cell 22:787-797); herpes thymidine kinase promoter (Wagner et al., 1981, Proc. Natl. Acad. Sci. U.S.A. 78: 1444-1445); promoter and regulatory sequences from the metallothionine gene (Prinster et al., 1982, Nature 296:39-42); and prokaryotic promoters such as the beta-lactamase promoter (Villa-Kamaroff et al., 1978, Proc. Natl. Acad. Sci. U.S.A. 75:3727-3731); or the tac promoter (DeBoer et al., 1983, Proc. Natl. Acad. Sci. U.S.A. 80:21-25). Also of interest are the following animal transcriptional control regions, which exhibit tissue specificity and have been utilized in transgenic animals: the elastase I gene control region that is active in pancreatic acinar cells (Swift et al., 1984, Cell 38:639-646; Ornitz et al., 1986, Cold Spring Harbor Symp. Quant. Biol. 50:399-409; MacDonald, 1987, Hepatology 7:425-515); the insulin gene control region that is active in pancreatic beta cells (Hanahan, 1985, Nature 315: 115-122); the immunoglobulin gene control region that is active in lymphoid cells (Grosschedl et al., 1984, Cell 38:647-658; Adames et al., 1985, Nature 318:533-538; Alexander et al., 1987, Mol. Cell. Biol. 7: 1436-1444); the mouse mammary tumor virus control region that is active in testicular, breast, lymphoid and mast cells (Leder et al., 1986, Cell 45:485-495); the albumin gene control region that is active in liver (Pinkert et al., 1987, Genes and Devel. 1:268-276); the alpha-feto-protein gene control region that is active in liver (Krumlauf et al., 1985, Mol. Cell. Biol. 5: 1639-1648; Hammer et al., 1987, Science 253:53-58); the alpha 1-antitrypsin gene control region that is active in liver (Kelsey et al., 1987, Genes and Devel. 1: 161-171); the beta-globin gene control region that is active in myeloid cells (Mogram et al, 1985, Nature 315:338-340; Kollias et al, 1986, Cell 46:89-94); the myelin basic protein gene control region that is active in oligodendrocyte cells in the brain (Readhead et al., 1987, Cell 48:703-712); the myosin light chain-2 gene control region that is active in skeletal muscle (Sani, 1985, Nature 314:283-286); and the gonadotropic releasing hormone gene control region that is active in the hypothalamus (Mason et al., 1986, Science 234: 1372-1378).

An enhancer sequence may be inserted into the vector to increase transcription of DNA encoding a component of the antibodies or antigen-binding fragments (e.g., light chain, heavy chain, or variable regions) by higher eukaryotes. Enhancers are cis-acting elements of DNA, usually about 10-300 bp in length, that act on the promoter to increase transcription. Enhancers are relatively orientation and position independent, having been found at positions both 5′ and 3′ to the transcription unit. Several enhancer sequences available from mammalian genes are known (e.g., globin, elastase, albumin, alpha-feto-protein and insulin). Typically, however, an enhancer from a virus is used. The SV40 enhancer, the cytomegalovirus early promoter enhancer, the polyoma enhancer, and adenovirus enhancers known in the art are exemplary enhancing elements for the activation of eukaryotic promoters. While an enhancer may be positioned in the vector either 5′ or 3′ to a coding sequence, it is typically located at a site 5′ from the promoter. A sequence encoding an appropriate native or heterologous signal sequence (leader sequence or signal peptide) can be incorporated into an expression vector, to promote extracellular secretion of the antibody or antigen-binding fragment as described above. The choice of signal peptide or leader depends on the type of host cells in which the antibody or antigen-binding fragment is to be produced, and a heterologous signal sequence can replace the native signal sequence. Examples of signal peptides are described above. Other signal peptides that are functional in mammalian host cells include the signal sequence for interleukin-7 (IL-7) described in U.S. Pat. No. 4,965,195; the signal sequence for interleukin-2 receptor described in Cosman et al., 1984, Nature 312:768; the interleukin-4 receptor signal peptide described in EP Patent No. 0367 566; the type I interleukin-1 receptor signal peptide described in U.S. Pat. No. 4,968,607; and the type II interleukin-1 receptor signal peptide described in EP Patent No. 0 460 846.

The expression vectors that are provided may be constructed from a starting vector such as a commercially available vector. Such vectors may or may not contain all of the desired flanking sequences. Where one or more of the flanking sequences described herein are not already present in the vector, they may be individually obtained and ligated into the vector. Methods used for obtaining each of the flanking sequences are well known to one skilled in the art. The expression vectors can be introduced into host cells to thereby produce proteins, including antibodies and antigen-binding fragments, encoded by nucleic acids as described herein.

In certain embodiments, nucleic acids encoding the different components of the anti-PAC1 antibodies or antigen-binding fragments of the invention may be inserted into the same expression vector. For instance, the nucleic acid encoding an anti-PAC1 antibody light chain or variable region can be cloned into the same vector as the nucleic acid encoding an anti-PAC1 antibody heavy chain or variable region. In such embodiments, the two nucleic acids may be separated by an internal ribosome entry site (IRES) and under the control of a single promoter such that the light chain and heavy chain are expressed from the same mRNA transcript. Alternatively, the two nucleic acids may be under the control of two separate promoters such that the light chain and heavy chain are expressed from two separate mRNA transcripts. In some embodiments, the nucleic acid encoding the anti-PAC1 antibody light chain or variable region is cloned into one expression vector and the nucleic acid encoding the anti-PAC1 antibody heavy chain or variable region is cloned into a second expression vector. In such embodiments, a host cell may be co-transfected with both expression vectors to produce complete antibodies or antigen-binding fragments of the invention.

After the vector has been constructed and the one or more nucleic acid molecules encoding the components of the antibodies and antigen-binding fragments described herein has been inserted into the proper site(s) of the vector or vectors, the completed vector(s) may be inserted into a suitable host cell for amplification and/or polypeptide expression. Thus, the present invention encompasses an isolated host cell or cell line comprising one or more expression vectors encoding the components of the anti-PAC1 antibodies or antigen-binding fragments described herein. The term “host cell” as used herein refers to a cell that has been transformed, or is capable of being transformed, with a nucleic acid and thereby expresses a gene of interest. The term includes the progeny of the parent cell, whether or not the progeny is identical in morphology or in genetic make-up to the original parent cell, so long as the gene of interest is present. A host cell that comprises an isolated nucleic acid of the invention, preferably operably linked to at least one expression control sequence (e.g. promoter or enhancer), is a “recombinant host cell.”

The transformation of an expression vector for an anti-PAC1 antibody or antigen-binding fragment into a selected host cell may be accomplished by well-known methods including transfection, infection, calcium phosphate co-precipitation, electroporation, microinjection, lipofection, DEAE-dextran mediated transfection, or other known techniques. The method selected will in part be a function of the type of host cell to be used. These methods and other suitable methods are well known to the skilled artisan, and are set forth, for example, in Sambrook et al., 2001.

A host cell, when cultured under appropriate conditions, synthesizes an antibody or antigen-binding fragment that can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity (such as glycosylation or phosphorylation) and ease of folding into a biologically active molecule.

Exemplary host cells include prokaryote, yeast, or higher eukaryote cells. Prokaryotic host cells include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacillus, such as B. subtilis and B. licheniformis, Pseudomonas, and Streptomyces. Eukaryotic microbes such as filamentous fungi or yeast are suitable cloning or expression hosts for recombinant polypeptides. Saccharomyces cerevisiae, or common baker's yeast, is the most commonly used among lower eukaryotic host microorganisms. However, a number of other genera, species, and strains are commonly available and useful herein, such as Pichia, e.g. P. pastoris, Schizosaccharomyces pombe; Kluyveromyces, Yarrowia; Candida; Trichoderma reesia; Neurospora crassa; Schwanniomyces, such as Schwanniomyces occidentalis; and filamentous fungi, such as, e.g., Neurospora, Penicillium, Tolypocladium, and Aspergillus hosts such as A. nidulans and A. niger.

Host cells for the expression of glycosylated antibodies and antigen-binding fragments can be derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection of such cells are publicly available, e.g., the L-1 variant of Autographa californica NPV and the Bm-5 strain of Bombyx mori NPV.

Vertebrate host cells are also suitable hosts, and recombinant production of antibodies and antigen-binding fragments from such cells has become routine procedure. Mammalian cell lines available as hosts for expression are well known in the art and include, but are not limited to, immortalized cell lines available from the American Type Culture Collection (ATCC), including but not limited to Chinese hamster ovary (CHO) cells, including CHOK1 cells (ATCC CCL61), DXB-11, DG-44, and Chinese hamster ovary cells/-DHFR (CHO, Urlaub et al., Proc. Natl. Acad. Sci. USA 77: 4216, 1980); monkey kidney CV1 line transformed by SV40 (COS-7, ATCC CRL 1651); human embryonic kidney line (293 or 293 cells subcloned for growth in suspension culture, (Graham et al., J. Gen Virol. 36: 59, 1977); baby hamster kidney cells (BHK, ATCC CCL 10); mouse sertoli cells (TM4, Mather, Biol. Reprod. 23: 243-251, 1980); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human hepatoma cells (Hep G2, HB 8065); mouse mammary tumor (MMT 060562, ATCC CCL51); TRI cells (Mather et al., Annals N.Y Acad. Sci. 383: 44-68, 1982); MRC 5 cells or FS4 cells; mammalian myeloma cells, and a number of other cell lines. In certain embodiments, cell lines may be selected through determining which cell lines have high expression levels and constitutively produce antibodies and antigen-binding fragments with PAC1 binding properties. In another embodiment, a cell line from the B cell lineage that does not make its own antibody but has a capacity to make and secrete a heterologous antibody can be selected. CHO cells are preferred host cells in some embodiments for expressing the anti-PAC1 antibodies and antigen-binding fragments of the invention.

Host cells are transformed or transfected with the above-described nucleic acids or vectors for production of anti-PAC1 antibodies or antigen-binding fragments and are cultured in conventional nutrient media modified as appropriate for inducing promoters, selecting transformants, or amplifying the genes encoding the desired sequences. In addition, novel vectors and transfected cell lines with multiple copies of transcription units separated by a selective marker are particularly useful for the expression of antibodies and antigen-binding fragments. Thus, the present invention also provides a method for producing an anti-PAC1 antibody or antigen-binding fragment thereof described herein comprising culturing a host cell comprising one or more expression vectors described herein in a culture medium under conditions permitting expression of the antibody or antigen-binding fragment encoded by the one or more expression vectors; and recovering the antibody or antigen-binding fragment from the culture medium or host cell.

The host cells used to produce the antibodies or antigen-binding fragments of the invention may be cultured in a variety of media. Commercially available media such as Ham's F10 (Sigma), Minimal Essential Medium (MEM, Sigma), RPMI-1640 (Sigma), and Dulbecco's Modified Eagle's Medium (DMEM, Sigma) are suitable for culturing the host cells. In addition, any of the media described in Ham et al., Meth. Enz. 58: 44, 1979; Barnes et al., Anal. Biochem. 102: 255, 1980; U.S. Pat. Nos. 4,767,704; 4,657,866; 4,927,762; 4,560,655; or 5,122,469; WO90103430; WO 87/00195; or U.S. Pat. Re. No. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinary skilled artisan.

Upon culturing the host cells, the antibody or antigen-binding fragment can be produced intracellularly, in the periplasmic space, or directly secreted into the medium. If the antibody or antigen-binding fragment is produced intracellularly, as a first step, the particulate debris, either host cells or lysed fragments, is removed, for example, by centrifugation or ultrafiltration. The antibody or antigen-binding fragment can be purified using, for example, hydroxyapatite chromatography, cation or anion exchange chromatography, or preferably affinity chromatography, using the antigen(s) of interest or protein A or protein G as an affinity ligand. Protein A can be used to purify proteins that include polypeptides that are based on human γ1, γ2, or γ4 heavy chains (Lindmark et al., J. Immunol. Meth. 62: 1-13, 1983). Protein G is recommended for all mouse isotypes and for human γ3 (Guss et al., EMBO J. 5: 1567-1575, 1986). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the protein comprises a CH3 domain, the Bakerbond ABX™ resin (J. T. Baker, Phillipsburg, N.J.) is useful for purification. Other techniques for protein purification such as ethanol precipitation, Reverse Phase HPLC, chromatofocusing, SDS-PAGE, and ammonium sulfate precipitation are also possible depending on the particular antibody or antigen-binding fragment to be recovered.

In certain embodiments, the invention provides a composition (e.g. a pharmaceutical composition) comprising one or a plurality of the anti-PAC1 antibodies or antigen-binding fragments of the invention (e.g. anti-PAC1 monoclonal antibodies or binding fragments thereof) together with pharmaceutically acceptable diluents, carriers, excipients, solubilizers, emulsifiers, preservatives, and/or adjuvants. The pharmaceutical compositions can be used in any of the methods described herein. Pharmaceutical compositions of the invention include, but are not limited to, liquid, frozen, and lyophilized compositions. “Pharmaceutically-acceptable” refers to molecules, compounds, and compositions that are non-toxic to human recipients at the dosages and concentrations employed and/or do not produce allergic or adverse reactions when administered to humans. In some embodiments, the pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. Methods and suitable materials for formulating molecules for therapeutic use are known in the pharmaceutical arts, and are described, for example, in REMINGTON'S PHARMACEUTICAL SCIENCES, 18th Edition, (A. R. Genrmo, ed.), 1990, Mack Publishing Company.

In some embodiments, the pharmaceutical composition of the invention comprises a standard pharmaceutical carrier, such as a sterile phosphate buffered saline solution, bacteriostatic water, and the like. A variety of aqueous carriers may be used, e.g., water, buffered water, 0.4% saline, 0.3% glycine and the like, and may include other proteins for enhanced stability, such as albumin, lipoprotein, globulin, etc., subjected to mild chemical modifications or the like.

Exemplary concentrations of the antibodies or antigen-binding fragments in the formulation may range from about 0.1 mg/ml to about 200 mg/ml or from about 0.1 mg/mL to about 50 mg/mL, or from about 0.5 mg/mL to about 25 mg/mL, or alternatively from about 2 mg/mL to about 10 mg/mL. An aqueous formulation of the antibody or antigen-binding fragment may be prepared in a pH-buffered solution, for example, at pH ranging from about 4.5 to about 6.5, or from about 4.8 to about 5.5, or alternatively about 5.0. Examples of buffers that are suitable for a pH within this range include acetate (e.g. sodium acetate), succinate (such as sodium succinate), gluconate, histidine, citrate and other organic acid buffers. The buffer concentration can be from about 1 mM to about 200 mM, or from about 10 mM to about 60 mM, depending, for example, on the buffer and the desired isotonicity of the formulation.

A tonicity agent, which may also stabilize the antibody or antigen-binding fragment, may be included in the formulation. Exemplary tonicity agents include polyols, such as mannitol, sucrose or trehalose. Preferably the aqueous formulation is isotonic, although hypertonic or hypotonic solutions may be suitable. Exemplary concentrations of the polyol in the formulation may range from about 1% to about 15% w/v.

A surfactant may also be added to the formulation to reduce aggregation of the formulated antibody or antigen-binding fragment and/or minimize the formation of particulates in the formulation and/or reduce adsorption. Exemplary surfactants include nonionic surfactants such as polysorbates (e.g. polysorbate 20 or polysorbate 80) or poloxamers (e.g. poloxamer 188). Exemplary concentrations of surfactant may range from about 0.001% to about 0.5%, or from about 0.005% to about 0.2%, or alternatively from about 0.004% to about 0.01% w/v.

In one embodiment, the formulation contains the above-identified agents (i.e. antibody or antigen-binding fragment, buffer, polyol and surfactant) and is essentially free of one or more preservatives, such as benzyl alcohol, phenol, m-cresol, chlorobutanol and benzethonium chloride. In another embodiment, a preservative may be included in the formulation, e.g., at concentrations ranging from about 0.1% to about 2%, or alternatively from about 0.5% to about 1%. One or more other pharmaceutically acceptable carriers, excipients or stabilizers such as those described in REMINGTON'S PHARMACEUTICAL SCIENCES, 18th Edition, (A. R. Genrmo, ed.), 1990, Mack Publishing Company, may be included in the formulation provided that they do not adversely affect the desired characteristics of the formulation.

Therapeutic formulations of the antibody or antigen-binding fragment are prepared for storage by mixing the antibody or antigen-binding fragment having the desired degree of purity with optional physiologically acceptable carriers, excipients or stabilizers (REMINGTON'S PHARMACEUTICAL SCIENCES, 18th Edition, (A. R. Genrmo, ed.), 1990, Mack Publishing Company), in the form of lyophilized formulations or aqueous solutions. Acceptable carriers, excipients, or stabilizers are nontoxic to recipients at the dosages and concentrations employed, and include those described above, such as buffers (e.g. phosphate, citrate, and other organic acids); antioxidants (e.g. ascorbic acid and methionine); preservatives (such as octadecyldimethylbenzyl ammonium chloride, hexamethonium chloride, benzalkonium chloride, benzethonium chloride, phenol, butyl or benzyl alcohol, alkyl parabens such as methyl or propyl paraben, catechol; resorcinol, cyclohexanol, 3-pentanol, and m-cresol); low molecular weight (e.g. less than about 10 residues) polypeptides; proteins (such as serum albumin, gelatin, or immunoglobulins); hydrophilic polymers (e.g. polyvinylpyrrolidone); amino acids (e.g. glycine, glutamine, asparagine, histidine, arginine, or lysine); monosaccharides, disaccharides, and other carbohydrates including glucose, mannose, maltose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants, such as polysorbates (e.g. polysorbate 20 or polysorbate 80) or poloxamers (e.g. poloxamer 188); or polyethylene glycol (PEG).

In one embodiment, a suitable formulation of the invention contains an isotonic buffer such as a phosphate, acetate, or TRIS buffer in combination with a tonicity agent, such as a polyol, sorbitol, sucrose or sodium chloride, which tonicifies and stabilizes. One example of such a tonicity agent is 5% sorbitol or sucrose. In addition, the formulation could optionally include a surfactant at 0.01% to 0.02% wt/vol, for example, to prevent aggregation or improve stability. The pH of the formulation may range from 4.5 to 6.5 or 4.5 to 5.5. Other exemplary descriptions of pharmaceutical formulations for antibodies and antigen-binding fragments may be found in US Patent Publication No. 2003/0113316 and U.S. Pat. No. 6,171,586, each of which is hereby incorporated by reference in its entirety.

The formulations to be used for in vivo administration must be sterile. The compositions of the invention may be sterilized by conventional, well-known sterilization techniques. For example, sterilization is readily accomplished by filtration through sterile filtration membranes. The resulting solutions may be packaged for use or filtered under aseptic conditions and lyophilized, the lyophilized preparation being combined with a sterile solution prior to administration.

The process of freeze-drying is often employed to stabilize polypeptides for long-term storage, particularly when the polypeptide is relatively unstable in liquid compositions. A lyophilization cycle is usually composed of three steps: freezing, primary drying, and secondary drying (see Williams and Polli, Journal of Parenteral Science and Technology, Volume 38, Number 2, pages 48-59, 1984). In the freezing step, the solution is cooled until it is adequately frozen. Bulk water in the solution forms ice at this stage. The ice sublimes in the primary drying stage, which is conducted by reducing chamber pressure below the vapor pressure of the ice, using a vacuum. Finally, sorbed or bound water is removed at the secondary drying stage under reduced chamber pressure and an elevated shelf temperature. The process produces a material known as a lyophilized cake. Thereafter the cake can be reconstituted prior to use. The standard reconstitution practice for lyophilized material is to add back a volume of pure water (typically equivalent to the volume removed during lyophilization), although dilute solutions of antibacterial agents are sometimes used in the production of pharmaceuticals for parenteral administration (see Chen, Drug Development and Industrial Pharmacy, Volume 18: 1311-1354, 1992).

Excipients have been noted in some cases to act as stabilizers for freeze-dried products (see Carpenter et al., Volume 74: 225-239, 1991). For example, known excipients include polyols (including mannitol, sorbitol and glycerol); sugars (including glucose and sucrose); and amino acids (including alanine, glycine and glutamic acid). In addition, polyols and sugars are also often used to protect polypeptides from freezing- and drying-induced damage and to enhance the stability during storage in the dried state. In general, sugars, in particular disaccharides, are effective in both the freeze-drying process and during storage. Other classes of molecules, including mono- and di-saccharides and polymers such as PVP, have also been reported as stabilizers of lyophilized products.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

Sustained-release preparations may be prepared. Suitable examples of sustained-release preparations include semipermeable matrices of solid hydrophobic polymers containing the antibody or antigen-binding fragment, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the Lupron Depot™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated polypeptides remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

The formulations of the invention may be designed to be short-acting, fast-releasing, long-acting, or sustained-releasing as described herein. Thus, the pharmaceutical formulations may also be formulated for controlled release or for slow release.

Specific dosages may be adjusted depending on the disease, disorder, or condition to be treated (e.g. episodic migraine, chronic migraine, or cluster headache), the age, body weight, general health conditions, sex, and diet of the subject, dose intervals, administration routes, excretion rate, and combinations of drugs.

The anti-PAC1 antibodies or antigen-binding fragments of the invention can be administered by any suitable means, including parenteral, subcutaneous, intravenous, intraperitoneal, intrapulmonary, and intranasal, and, if desired for local treatment, intralesional administration. Parenteral administration includes intravenous, intraarterial, intraperitoneal, intramuscular, intradermal or subcutaneous administration. In addition, the antibody or antigen-binding fragment is suitably administered by pulse infusion, particularly with declining doses of the antibody or antigen-binding fragment. Preferably, the dosing is given by injections, most preferably intravenous or subcutaneous injections, depending in part on whether the administration is brief or chronic. Other administration methods are contemplated, including topical, particularly transdermal, transmucosal, rectal, oral or local administration e.g. through a catheter placed close to the desired site. The antibody or antigen-binding fragment of the invention may be administered in a physiological solution at a dose ranging between 0.01 mg/kg to 100 mg/kg at a frequency ranging from daily to weekly to monthly.

The anti-PAC1 antibodies and antigen-binding fragments described herein are useful for treating or ameliorating a condition associated with the biological activity of the PAC1 receptor in a patient in need thereof. Thus, anti-PAC1 antibodies and antigen-binding fragments of the invention for use in methods of treatment are disclosed herein. The term “patient” includes human patients and is used interchangeably with the term “subject.” As used herein, the term “treating” or “treatment” is an intervention performed with the intention of preventing the development or altering the pathology of a disorder. Accordingly, “treatment” refers to both therapeutic treatment and prophylactic or preventative measures. Those in need of treatment include those already diagnosed with or suffering from the disorder or condition as well as those in which the disorder or condition is to be prevented. “Treatment” includes any indicia of success in the amelioration of an injury, pathology or condition, including any objective or subjective parameter such as abatement, remission, diminishing of symptoms, or making the injury, pathology or condition more tolerable to the patient, slowing in the rate of degeneration or decline, making the final point of degeneration less debilitating, or improving a patient's physical or mental well-being. The treatment or amelioration of symptoms can be based on objective or subjective parameters, including the results of a physical examination, self-reporting by a patient, neuropsychiatric exams, and/or a psychiatric evaluation.

Accordingly, in some embodiments, the present invention provides a method for treating or preventing a condition associated with the biological activity of the PAC1 receptor (e.g. a condition associated with PACAP-induced activation of the PAC1 receptor) in a patient in need thereof, comprising administering to the patient an effective amount of an anti-PAC1 antibody or antigen-binding fragment thereof described herein. The PACAP/PAC1 signaling pathway has been implicated in various physiological processes, including cardiovascular function, metabolic and endocrine function, inflammation, stress response, regulation of vasomotor tone, and regulation of the autonomic nervous system, particularly the balance between the sympathetic and parasympathetic systems. See, e.g., Tanida et al., Regulatory Peptides, Vol. 161: 73-80, 2010; Moody et al., Curr. Opin. Endocrinol. Diabetes Obes., Vol. 18: 61-67, 2011; and Hashimoto et al., Current Pharmaceutical Design, Vol. 17: 985-989, 2011. Conditions associated with aberrant or overactivation of the PACAP/PAC1 signaling pathway include, but are not limited to, headache conditions, such as migraine, cluster headache, tension-type headache, hemiplegic migraine, and retinal migraine; inflammatory skin conditions; chronic pain, such as neuropathic pain; anxiety disorders; irritable bowel syndrome; and vasomotor symptoms, such as hot flashes, facial flushing, sweating, and night sweats. Thus, the anti-PAC1 antibodies and antigen-binding fragments thereof of the invention can be administered to patients to prevent, ameliorate, or treat any of these conditions or disorders or other conditions associated with aberrant or excessive PAC1 receptor biological activity. In certain embodiments, the present invention provides methods for treating or preventing a headache condition (e.g. episodic migraine, chronic migraine, cluster headache, tension-type headache, hemiplegic migraine, and retinal migraine) in a patient in need thereof comprising administering to the patient an effective amount of an anti-PAC1 antibody or antigen-binding fragment thereof as described herein. In some embodiments, the present invention provides a method for inhibiting activation of the human PAC1 receptor in a patient having a headache condition comprising administering to the patient an effective amount of an anti-PAC1 antibody or antigen-binding fragment thereof described herein. In one embodiment, the patient has a migraine headache condition, such as episodic migraine or chronic migraine. In another embodiment, the patient has a cluster headache condition.

An “effective amount” is generally an amount sufficient to reduce the severity and/or frequency of symptoms, eliminate the symptoms and/or underlying cause, prevent the occurrence of symptoms and/or their underlying cause, and/or improve or remediate the damage that results from or is associated with a particular condition. In some embodiments, the effective amount is a therapeutically effective amount or a prophylactically effective amount. A “therapeutically effective amount” is an amount sufficient to remedy a disease state or symptom(s), particularly a state or symptom(s) associated with the disease state, or otherwise prevent, hinder, retard or reverse the progression of the disease state or any other undesirable symptom associated with the disease in any way whatsoever (i.e. that provides “therapeutic efficacy”). A “prophylactically effective amount” is an amount of an antibody or antigen-binding fragment that, when administered to a subject, will have the intended prophylactic effect, e.g., preventing or delaying the onset (or reoccurrence) of the condition, or reducing the likelihood of the onset (or reoccurrence) of the condition. The full therapeutic or prophylactic effect does not necessarily occur by administration of one dose, and may occur only after administration of a series of doses. Thus, a therapeutically or prophylactically effective amount may be administered in one or more administrations.

In certain embodiments, the present invention provides methods for inhibiting vasodilation in a patient in need thereof comprising administering to the patient an effective amount of an anti-PAC1 antibody or antigen-binding fragment thereof described herein. Ligands of the PAC1 receptor, such as PACAP38 and VIP, are potent vasodilators, and blocking the binding of these ligands to the PAC1 receptor can inhibit vasodilation and ameliorate conditions associated with aberrant or excessive vasodilation, such as headache conditions, hot flashes, and flushing. In one embodiment, the patient has a headache condition, such as migraine or cluster headache. In another embodiment, the patient has vasomotor symptoms (e.g. hot flashes, facial flushing, sweating, or night sweats). In a related embodiment, the patient has vasomotor symptoms associated with menopause.

In some embodiments of the methods of the invention, the headache condition to be treated, prevented or ameliorated is migraine. Thus, the present invention includes a method for treating, preventing, or ameliorating migraine in a patient in need thereof comprising administering to the patient an effective amount of an anti-PAC1 antibody or antigen-binding fragment thereof described herein. Migraine headaches are recurrent headaches lasting about 4 to about 72 hours that are characterized by unilateral, pulsating, and/or moderate to severe pain and/or pain that is exacerbated by physical activity. Migraine headaches are often accompanied by nausea, vomiting, and/or sensitivity to light (photophobia), sound (phonophobia), or smell. In some patients, an aura precedes the onset of the migraine headache. The aura is typically a visual, sensory, language, or motor disturbance that signals the headache will soon occur. The methods described herein prevent, treat, or ameliorate one or more symptoms of migraine headaches with and without aura in human patients.

PACAP38, through activation of its receptors, induces vasodilation, particularly vasodilation of the dura vasculature (Schytz et al., Neurotherapeutics, Vol. 7(2):191-196, 2010). The PACAP38/PAC1 receptor signaling cascade, in particular, has been implicated in migraine pathophysiology (Amin et al., Brain, Vol. 137: 779-794, 2014). Infusion of PACAP38, which has a higher affinity for the PAC1 receptor than the VPAC1 and VPAC2 receptors, causes migraine-like headache in migraine patients (Schytz et al., Brain 132:16-25, 2009; Amin et al., Brain, Vol. 137: 779-794, 2014; Guo et al., Cephalalgia, Vol. 37:125-135, 2017). In addition, PACAP38 levels are elevated in cranial circulation in patients experiencing a migraine attack, and the PACAP38 levels are reduced following treatment of the migraine symptoms with triptans (Tuka et al., Cephalalgia, Vol. 33, 1085-1095, 2013; Zagami et al., Ann. Clin. Transl. Neurol., Vol. 1: 1036-1040, 2014). These reports suggest that endogenous release of PACAP38 is an important trigger of migraine headache and its effects are primarily mediated through activation of the PAC1 receptor.

In some embodiments, the patients to be treated according to the methods of the invention have, suffer from, or are diagnosed with episodic migraine. Episodic migraine is diagnosed when patients with a history of migraine (e.g. at least five lifetime attacks of migraine headache) have 14 or fewer migraine headache days per month. A “migraine headache day” includes any calendar day during which a patient experiences the onset, continuation, or recurrence of a “migraine headache” with or without aura lasting greater than 30 minutes. A “migraine headache” is a headache associated with nausea or vomiting or sensitivity to light or sound and/or a headache characterized by at least two of the following pain features: unilateral pain, throbbing pain, moderate to severe pain intensity, or pain exacerbated by physical activity. In certain embodiments, patients having, suffering from, or diagnosed with episodic migraine have at least four, but less than 15 migraine headache days per month on average. In related embodiments, patients having, suffering from, or diagnosed with episodic migraine have fewer than 15 headache days per month on average. As used herein, a “headache day” is any calendar day in which the patient experiences a migraine headache as defined herein or any headache that lasts greater than 30 minutes or requires acute headache treatment.

In certain embodiments, the patients to be treated according to the methods of the invention have, suffer from, or are diagnosed with chronic migraine. Chronic migraine is diagnosed when migraine patients (i.e. patients with at least five lifetime attacks of migraine headache) have 15 or more headache days per month and at least 8 of the headache days are migraine headache days. In some embodiments, patients having, suffering from, or diagnosed with chronic migraine have 15 or more migraine headache days per month on average. In certain embodiments of the methods described herein, administration of an anti-PAC1 antibody or antigen-binding fragment of the invention prevents, reduces, or delays the progression of episodic migraine to chronic migraine in the patient.

In some embodiments, the present invention provides a method for treating, preventing, or ameliorating cluster headache in a patient in need thereof comprising administering to the patient an effective amount of an anti-PAC1 antibody or antigen-binding fragment thereof described herein. Cluster headache is a condition that involves, as its most prominent feature, recurrent, severe headaches on one side of the head, typically around the eye (see Nesbitt et al., BMJ, Vol. 344:e2407, 2012). Cluster headaches often occur periodically: spontaneous remissions interrupt active periods of pain. Cluster headaches are often accompanied by cranial autonomic symptoms, such as tearing, nasal congestion, ptosis, pupil constriction, facial blushing, sweating, and swelling around the eye, often confined to the side of the head with the pain. The average age of onset of cluster headache is −30-50 years. It is more prevalent in males with a male to female ratio of about 2.5:1 to about 3.5:1. Sphenopalatine ganglion (SPG) stimulation has been used for the treatment of cluster headache. A neurostimulation system, which delivers low-level (but high frequency, physiologic-blocking) electrical stimulation to the SPG, has demonstrated efficacy in relieving the acute debilitating pain of cluster headache in a recent clinical trial (see Schoenen J, et al., Cephalalgia, Vol. 33(10):816-30, 2013). In view of this evidence and because PACAP is one of the major neurotransmitters in SPG, inhibition of PACAP/PAC1 signaling with an anti-PAC1 antibody or antigen-binding fragment thereof described herein is expected to have efficacy in treating cluster headache in humans.

Other conditions associated with the PACAP/PAC1 signaling pathway that may be treated according to the methods of the invention include, but are not limited to, inflammatory skin conditions, such as rosacea (see U.S. Patent Publication No. 20110229423), chronic pain syndromes, such as neuropathic pain (see Jongsma et al., Neuroreport, Vol. 12: 2215-2219, 2001; Hashimoto et al., Annals of the New York Academy of Sciences, Vol. 1070: 75-89, 2006), tension-type headaches, hemiplegic migraine, retinal migraine, anxiety disorders, such as posttraumatic stress disorder (see Hammack and May, Biol. Psychiatry, Vol. 78(3):167-177, 2015), irritable bowel syndrome, and vasomotor symptoms (e.g. hot flashes, facial flushing, sweating, and night sweats), such as those associated with menopause. In one embodiment, the condition to be treated by administering an anti-PAC1 antibody or antigen-binding fragment thereof of the invention is chronic pain. In another embodiment, the condition to be treated by administering an anti-PAC1 antibody or antigen-binding fragment thereof of the invention is neuropathic pain.

In any of the methods described herein, the treatment can comprise prophylactic treatment. Prophylactic treatment refers to treatment designed to be taken before the onset of a condition or an attack (e.g. before a migraine attack or onset of a cluster headache episode) to reduce the frequency, severity, and/or length of the symptoms (e.g. migraine or cluster headaches) in the patient.

In some embodiments, the methods of the invention for treating or preventing a headache condition in a patient comprise administering to the patient an anti-PAC1 antibody or antigen-binding fragment thereof described herein in combination with one or more agents suitable for the acute or prophylactic treatment of migraine headache or other headache disorder described herein. The term “combination therapy” as used herein encompasses the administration of the two compounds (e.g. anti-PAC1 antibody and additional agent) in a sequential manner (i.e. each compound is administered at a different time in any order) as well as administration of the two compounds in a substantially simultaneous manner. Substantially simultaneous administration includes concurrent administration and can be accomplished by administering a single formulation comprising both compounds (e.g. a single formulation comprising a fixed ratio of both compounds or a pre-filled syringe having a fixed ratio of each compound) or concurrently administering separate formulations containing each of the compounds. Thus, in certain embodiments, the methods of the invention comprise administering an anti-PAC1 antibody or antigen-binding fragment thereof described herein with a second headache therapeutic agent.

In certain embodiments, the second headache therapeutic agent may be an acute headache therapeutic agent used for the acute treatment of headaches or migraines. In some embodiments, the acute headache therapeutic agent is a serotonin (5-hydroxytryptamine; 5-HT) receptor agonist, for example a 5HT1 receptor agonist. The acute headache therapeutic agent can be an agonist of the 5HT_{1B}, 5HT_{1D} and/or 5HT_{1F} serotonin receptors. Such serotonin receptor agonists include, but are not limited to, triptans (e.g., almotriptan, frovatriptan, rizatriptan, sumatriptan, naratriptan, eletriptan, and zolmitriptan), ergotamines (e.g., dihydroergotamine and ergotamine tartrate), and 5HT_{1F}-selective serotonin receptor agonists, such as lasmiditan. Other suitable acute headache therapeutic agents include non-steroidal anti-inflammatory drugs (e.g., acetylsalicylic acid, ibuprofen, naproxen, indomethacin, and diclofenac), and opioids (e.g., codeine, morphine, hydrocodone, fentanyl, meperidine, and oxycodone). In one embodiment, the acute headache therapeutic agent administered in combination with an anti-PAC1 antibody or antigen-binding fragment of the invention is a triptan. In another embodiment, the acute headache therapeutic agent administered in combination with an anti-PAC1 antibody or antigen-binding fragment of the invention is an ergotamine. In yet another embodiment, the acute headache therapeutic agent administered in combination with an anti-PAC1 antibody or antigen-binding fragment of the invention is a non-steroidal anti-inflammatory drug. In still another embodiment, the acute headache therapeutic agent administered in combination with an anti-PAC1 antibody or antigen-binding fragment of the invention is an opioid.

In some embodiments, the second headache therapeutic agent is a prophylactic headache therapeutic agent used for the prophylactic treatment of headaches or migraines. In one embodiment, the prophylactic headache therapeutic agent is an antiepileptic, such as divalproex, sodium valproate, valproic acid, topiramate, or gabapentin. In another embodiment, the prophylactic headache therapeutic agent is a beta-blocker, such as propranolol, timolol, atenolol, metoprolol, or nadolol. In yet another embodiment, the prophylactic headache therapeutic agent is an anti-depressant, such as a tricyclic antidepressant (e.g. amitriptyline, nortriptyline, doxepin, and fluoxetine). In still another embodiment, the prophylactic headache therapeutic agent is onabotulinum toxin A.

In certain embodiments, the methods of the invention comprise administering an anti-PAC1 antibody or antigen-binding fragment thereof described herein with an antagonist of the calcitonin gene-related peptide (CGRP) signaling pathway (i.e. inhibits the activation or signaling of the CGRP receptor by the CGRP ligand). For example, the anti-PAC1 antibody or antigen-binding fragment of the invention can be administered in combination with a CGRP pathway antagonist to treat or prevent a headache condition (e.g. migraine or cluster headache) in a patient in need thereof. In some embodiments, the CGRP pathway antagonist is an antagonist of the human CGRP receptor. CGRP receptor antagonists include small molecule inhibitors of the CGRP receptor, such as those described in U.S. Patent Publication No. 20060142273 and U.S. Pat. Nos. 7,842,808; 7,772,244; 7,754,732; 7,569,578; 8,685,965; 8,569,291; 8,377,955; 8,372,859; 8,143,266; 7,947,677; and 7,625,901, all of which are hereby incorporated by reference in their entireties. CGRP receptor antagonists can also include peptide antagonists of the receptor, such as those described in U.S. Pat. No. 8,168,592, which is hereby incorporated by reference in its entirety. In certain embodiments, the CGRP receptor antagonist to be administered with the anti-PAC1 antibodies and antigen-binding fragments of the invention is a monoclonal antibody that specifically binds to the human CGRP receptor, such as the antibodies described in U.S. Pat. No. 9,102,731 and U.S. Patent Publication No. 20160311913, both of which are hereby incorporated by reference in their entireties. In one particular embodiment of the methods of the invention, an anti-PAC1 antibody or antigen-binding fragment thereof described herein is administered in combination with an anti-CGRP receptor monoclonal antibody comprising a light chain variable region comprising the sequence of SEQ ID NO: 502 (sequence provided below) and a heavy chain variable region comprising the sequence of SEQ ID NO: 503 (sequence provided below) to treat or prevent a headache condition (e.g. migraine or cluster headache) in a patient. In another particular embodiment of the methods of the invention, the anti-CGRP receptor monoclonal antibody administered in combination with the anti-PAC1 antibody or antigen-binding fragment of the invention to treat or prevent a headache condition (e.g. migraine or cluster headache) in a patient is erenumab.

Light chain variable region sequence for exemplary anti-CGRP receptor monoclonal antibody:

(SEQ ID NO: 502)
QSVLTQPPSV SAAPGQKVTI SCSGSSSNIG NNYVSWYQQL
PGTAPKLLIY DNNKRPSGIP DRFSGSKSGT STTLGITGLQ
TGDEADYYCG TWDSRLSAVV FGGGTKLTVL

Heavy chain variable region sequence for exemplary anti-CGRP receptor monoclonal antibody:

(SEQ ID NO: 503)
QVQLVESGGG VVQPGRSLRL SCAASGFTFS SFGMHWVRQA
PGKGLEWVAV ISFDGSIKYS VDSVKGRFTI SRDNSKNTLF
LQMNSLRAED TAVYYCARDR LNYYDSSGYY HYKYYGMAVW
GQGTTVTVSS

In some embodiments, the CGRP pathway antagonist to be administered with the anti-PAC1 antibody or antigen-binding fragment of the invention to treat or prevent a headache condition (e.g. migraine or cluster headache) in a patient is an antagonist of the CGRP ligand. A CGRP ligand antagonist can be a decoy or soluble CGRP receptor or other protein that binds to the CGRP ligand, such as an anti-CGRP ligand antibody. Anti-CGRP ligand antibodies are known in the art and are described, for example, in WO 2007/054809; WO 2007/076336; WO 2011/156324; and WO 2012/162243, all of which are hereby incorporated by reference in their entireties. In certain embodiments, the CGRP ligand antagonist to be administered with the anti-PAC1 antibodies and antigen-binding fragments of the invention is a monoclonal antibody that specifically binds to human α-CGRP and/or human I3-CGRP. In one embodiment, the anti-CGRP ligand antibody is fremanezumab. In another embodiment, the anti-CGRP ligand antibody is galcanezumab. In yet another embodiment, the anti-CGRP ligand antibody is eptinezumab.

The present invention also includes the use of anti-PAC1 antibodies and antigen-binding fragments in any of the methods disclosed herein. For instance, in certain embodiments, the present invention provides an anti-PAC1 antibody or antigen-binding fragment thereof described herein for use in a method for treating or preventing a headache condition in a patient in need thereof. In some such embodiments, the headache condition is migraine. The migraine may be episodic migraine or chronic migraine. In other embodiments, the headache condition is cluster headache. In some embodiments, the method for treating or preventing a headache condition comprises administering a second headache therapeutic agent in combination with the anti-PAC1 antibody or antigen-binding fragment thereof. In one embodiment, the second headache therapeutic agent is an acute headache therapeutic agent, such as a 5HT_{1B}, 5HT_{1D} and/or 5HT_{1F} serotonin receptor agonist (e.g. a triptan or ergotamine), a non-steroidal anti-inflammatory drug, or an opioid. In another embodiment, the second headache therapeutic agent is a prophylactic headache therapeutic agent, such as an antiepileptic, a beta-blocker, an anti-depressant, onabotulinum toxin A, or a CGRP pathway antagonist. In some embodiments, the CGRP pathway antagonist is a human CGRP receptor antagonist. In one particular embodiment, the human CGRP receptor antagonist is a monoclonal antibody that specifically binds to the human CGRP receptor, such as erenumab. In other embodiments, the CGRP pathway antagonist is an antagonist of the CGRP ligand, such as a monoclonal antibody that specifically binds to human α-CGRP and/or human β-CGRP. In certain embodiments, the anti-CGRP ligand antibody is fremanezumab, galcanezumab, or eptinezumab.

In some embodiments, the present invention provides an anti-PAC1 antibody or antigen-binding fragment described herein for use in a method for inhibiting vasodilation in a patient in need thereof. In such embodiments, the patient may be diagnosed with or have a headache condition, such as migraine (e.g. episodic or chronic migraine) or cluster headache. In other embodiments, the present invention provides an anti-PAC1 antibody or antigen-binding fragment described herein for use in a method for inhibiting activation of human PAC1 receptor in a patient having a headache condition. The headache condition may be migraine (e.g. episodic or chronic migraine) or cluster headache.

The use of anti-PAC1 antibodies or antigen-binding fragments thereof for preparation of medicaments for administration according to any of the methods disclosed herein is specifically contemplated. For example, in some embodiments, the present invention encompasses the use of an anti-PAC1 antibody or antigen-binding fragment described herein in the preparation of a medicament for treating or preventing a headache condition in a patient in need thereof. In some such embodiments, the headache condition is migraine. The migraine may be episodic migraine or chronic migraine. In other embodiments, the headache condition is cluster headache. In certain embodiments, the anti-PAC1 antibody or antigen-binding fragment thereof is formulated for administration with a second headache therapeutic agent. In one embodiment, the second headache therapeutic agent is an acute headache therapeutic agent, such as a 5HT_{1B}, 5HT_{1D} and/or 5HT_{1F} serotonin receptor agonist (e.g. a triptan or ergotamine), a non-steroidal anti-inflammatory drug, or an opioid. In another embodiment, the second headache therapeutic agent is a prophylactic headache therapeutic agent, such as an antiepileptic, a beta-blocker, an anti-depressant, onabotulinum toxin A, or a CGRP pathway antagonist. In some embodiments, the CGRP pathway antagonist is a human CGRP receptor antagonist. In one particular embodiment, the human CGRP receptor antagonist is a monoclonal antibody that specifically binds to the human CGRP receptor, such as erenumab. In other embodiments, the CGRP pathway antagonist is an antagonist of the CGRP ligand, such as a monoclonal antibody that specifically binds to human α-CGRP and/or human β-CGRP. In certain embodiments, the anti-CGRP ligand antibody is fremanezumab, galcanezumab, or eptinezumab.

In some embodiments, the present invention includes the use of an anti-PAC1 antibody or antigen-binding fragment described herein in the preparation of a medicament for inhibiting vasodilation in a patient in need thereof. In such embodiments, the patient may be diagnosed with or have a headache condition, such as migraine (e.g. episodic or chronic migraine) or cluster headache. In other embodiments, the present invention includes the use of an anti-PAC1 antibody or antigen-binding fragment described herein in the preparation of a medicament for inhibiting activation of human PAC1 receptor in a patient having a headache condition. The headache condition may be migraine (e.g. episodic or chronic migraine) or cluster headache.

The anti-PAC1 antibodies and antigen-binding fragments of the invention are also useful for detecting human PAC1 in biological samples and identification of cells or tissues that express human PAC1. For instance, the anti-PAC1 antibodies and antigen-binding fragments can be used in diagnostic assays, e.g., immunoassays to detect and/or quantify PAC1 expressed in a tissue or cell. In addition, the anti-PAC1 antibodies and antigen-binding fragments described herein can be used to inhibit PAC1 from forming a complex with PACAP, thereby modulating the biological activity of PAC1 in a cell or tissue. Such biological activity includes elevation of intracellular cAMP and vasodilation.

The anti-PAC1 antibodies and antigen-binding fragments described herein can be used for diagnostic purposes to detect, diagnose, or monitor diseases and/or conditions associated with PAC1, including migraine, cluster headache, and anxiety disorders, such as posttraumatic stress disorder. Also provided are methods for the detection of the presence of PAC1 in a sample using classical immunohistological methods known to those of skill in the art (e.g., Tijssen, 1993, Practice and Theory of Enzyme Immunoassays, Vol 15 (Eds R. H. Burdon and P. H. van Knippenberg, Elsevier, Amsterdam); Zola, 1987, Monoclonal Antibodies: A Manual of Techniques, pp. 147-158 (CRC Press, Inc.); Jalkanen et al., 1985, J. Cell. Biol. 101:976-985; Jalkanen et al., 1987, J. Cell Biol. 105:3087-3096). Examples of methods useful in the detection of the presence of PAC1 include immunoassays, such as the enzyme linked immunosorbent assay (ELISA) and the radioimmunoassay (MA), using the anti-PAC1 antibodies and antigen-binding fragments described herein. The detection of PAC1 can be performed in vivo or in vitro.

For diagnostic applications, the anti-PAC1 antibody or antigen-binding fragment can be labeled with a detectable labeling group. Suitable labeling groups include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent groups (e.g., FITC, rhodamine, lanthanide phosphors), enzymatic groups (e.g., horseradish peroxidase, β-galactosidase, luciferase, alkaline phosphatase), chemiluminescent groups, biotinyl groups, or predetermined polypeptide epitopes recognized by a secondary reporter (e.g., leucine zipper pair sequences, binding sites for secondary antibodies, metal binding domains, epitope tags). In some embodiments, the labeling group is coupled to the antibody or antigen-binding fragment via spacer arms of various lengths to reduce potential steric hindrance. Various methods for labeling proteins are known in the art and may be used.

In another embodiment, the anti-PAC1 antibodies and antigen-binding fragments described herein can be used to identify a cell or cells that express PAC1. In a specific embodiment, the antibody or antigen-binding fragment is labeled with a labeling group and the binding of the labeled antibody or antigen-binding fragment to PAC1 is detected. The antibodies or antigen-binding fragments can also be used in immunoprecipitation assays in biological samples. In a further specific embodiment, the binding of the antibody or antigen-binding fragment to PAC1 is detected in vivo. In a further specific embodiment, the antibody or antigen-binding fragment is isolated and measured using techniques known in the art. See, for example, Harlow and Lane, 1988, Antibodies: A Laboratory Manual, New York: Cold Spring Harbor (ed. 1991 and periodic supplements); John E. Coligan, ed., 1993, Current Protocols In Immunology New York: John Wiley & Sons.

The following examples, including the experiments conducted and the results achieved, are provided for illustrative purposes only and are not to be construed as limiting the scope of the appended claims.

### EXAMPLES

### Example 1. Crystal Structure-Guided Design of Human PAC1 Antibodies

The crystal structure of a complex between the extracellular domain (ECD) of human PAC1 and the Fab fragment of a human anti-PAC1 neutralizing antibody (29G4v9) was determined. The human PAC1 ECD and anti-PAC1 Fab were separately purified and then complexed together in 1:1 molar ratio. The sample was subsequently run over gel filtration column equilibrated in 20 mM TRIS pH 7.5, 50 mM NaCl, 5 mM EDTA and concentrated to 35 mg/ml and filtered.

The sequences for the heavy chain (comprising the variable region (VH), CH1 constant region, upper hinge, and caspase III cleavage site) and the light chain (comprising the variable region (VL) and CL constant region) of the Fab fragment are listed below. The sequence of the human PAC1 ECD construct is provided below and contained amino acids 26 to 143 of human PAC1 (SEQ ID NO: 1) minus the region between amino acids 89-109.

Amino acid sequence for heavy chain of 29G4v9 Fab (comprised of VH region (amino acids: 1-120); CH1 region (amino acids: 121-218); upper hinge region (amino acids 219-221), and caspase III cleavage site (222-226):

(SEQ ID NO: 2)
QVQLVESGGG VVQPGRSLRL SCAASGFTFS RFAMHWVRQA
PGKGLEWVAV ISYDGGNKYY AESVKGRFTI SRDNSKNTLY
LQMNSLRAED TALFYCARGY DVLTGYPDYW GQGTLVTVSS
ASTKGPSVFP LAPCSRSTSE STAALGCLVK DYFPEPVTVS
WNSGALTSGV HTFPAVLQSS GLYSLSSVVT VPSSNFGTQT
YTCNVDHKPS NTKVDKTVER KGDEVD

Amino acid sequence for light chain of 29G4v9 Fab (comprised of VL region (amino acids: 1-108) and CL region (amino acids: 109-214):

(SEQ ID NO: 3)
DIQLTQSPSF LSASVGDRVT ITCRASQSIG RSLHWYQQKP
GKAPKLLIKY ASQSLSGVPS RFSGSGSGTE FTLTISSLQP
EDFATYYCHQ SSRLPFTFGP GTKVDIKRTV AAPSVFIFPP
SDEQLKSGTA SVVCLLNNFY PREAKVQWKV DNALQSGNSQ
ESVTEQDSKD STYSLSSTLT LSKADYEKHK VYACEVTHQG
LSSPVTKSFN RGEC

Amino acid sequence of the human PAC1 ECD construct:

(SEQ ID NO: 4)
GSMAHSDGIF KKEQAMCLEK IQRANELMGF NDSSPGCPGM
WDNITCWKPA HVGEMVLVSC PELFRIFNPD QDMGVVSRNC
TEDGWSEPFP HYFDACGFDE YESET

Purified human PAC1 ECD and the anti-PAC1 Fab were initially co-crystallized using sitting drop vapor diffusion method with commercially available screens. The crystallized condition (Qiagen MPD Suite Screen #61 (134561)) was further expanded using hanging drop vapor diffusion method. Protein and crystallization buffer (0.1M Citric Acid pH 4.0, 40% MPD) were mixed 1:1 in 1111 hanging drops over a reservoir solution of crystallization buffer at 4° C. Rod-shaped crystals formed within several weeks.

The crystal was equilibrated in the crystallization buffer as cryo-protectant, and was frozen in liquid nitrogen for shipment to Lawrence Berkeley National Laboratories for data collection. The data set was collected at the Advanced Light Source Synchrotron Beamline 5.0.2 on an ADSC-Q315r CCD Detector (λ=1.000 Å). The data were integrated and scaled using HKL2000 (Otwinowski and Minor, Methods Enzymology, Vol. 276, 307-326, 1997) and were 99.8% complete to 2.00 Å with R_{merge} of 0.081 (98.0% complete last shell 2.07-2.00 Å with I/σ=2.35). The crystals belong to the orthorhombic space group P21212 with unit cell dimensions of a=65.2 Å, b=177.9 Å, c=53.8 Å, α=90°, β=90°, γ=90°. The crystal structure was solved by molecular replacement using PhaserMR (Winn et al., Acta Crystallogr., Sect. D: Biol. Crystallogr., Vol. 67: 235-242, 2011). A proprietary Fab structure was used as the first search model to solve the anti-PAC1 Fab component. Subsequent molecular replacement used PDBID: 2JOD (Sun et al., Proc. Natl. Acad. Sci. USA, Vol. 104: 7875, 2007), a human PAC1 ECD NMR structure, as starting model to solve the human PAC1 ECD component. There is one ECD molecule and one Fab molecule in the asymmetric unit. The structure was refined using both Refmac5 (Winn et al., Acta Crystallogr., Sect. D: Biol. Crystallogr., Vol. 67: 235-242, 2011; Murshudov et al., Acta Crystallogr., Sect. D: Biol. Crystallogr., Vol. 67: 355-367, 2011) and Phenix.refine (Adams et al., Acta Crystallogr., Sect. D: Biol. Crystallogr., Vol. 66: 213-221, 2010), and model building was performed using the graphics program Coot (Emsley and Cowtan, Acta Crystallogr., Sect. D: Biol. Crystallogr., Vol. 2004, 60: 2126-2132, 2004).

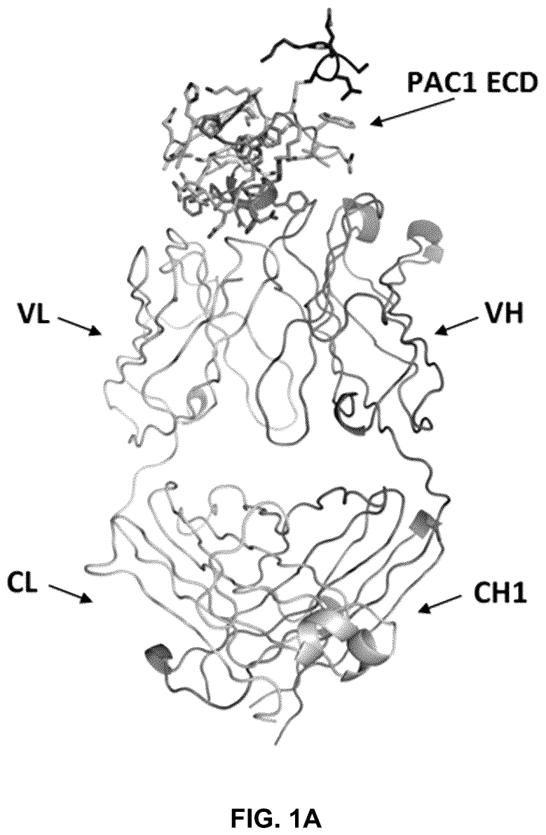

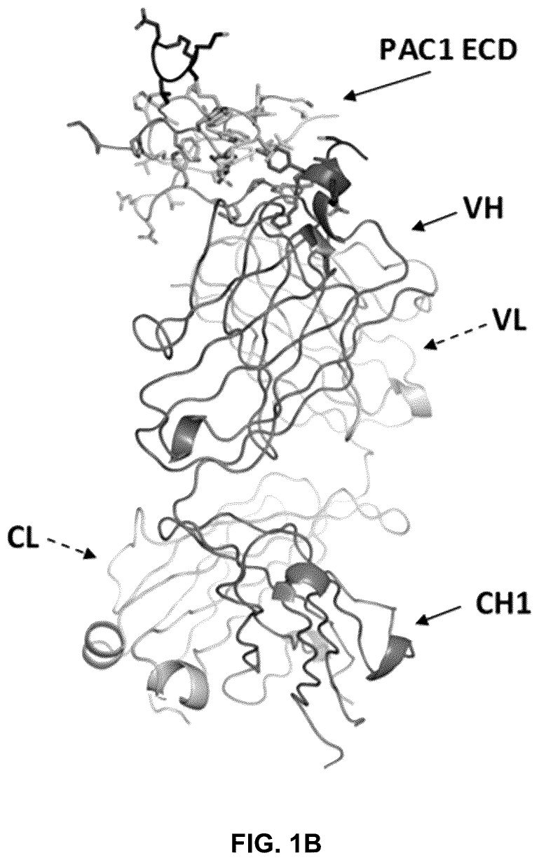

The structure of the human PAC1 ECD: anti-PAC1 Fab complex was refined to 2.00 Å with an R-factor of 20% and R_{free} of 23%. A front view and side view of the structure of the complex is shown in FIGS. 1A and 1B, respectively. The interaction between the 29G4v9 Fab and the PAC1 ECD is comprised of hydrophobic, electrostatic and hydrogen bond interactions. The interaction between the 29G4v9 Fab and the PAC1 ECD has buried surface area (1613 Å 2) and shape complementarity (0.695) values that are typical for antibody-antigen interactions. All amino acids in the PAC1 ECD that contained at least one non-hydrogen atom at a distance of 5 Å or less from a non-hydrogen atom in the 29G4v9 Fab were determined to be the core interface amino acids in the PAC1 ECD. Distances of the atoms were calculated with the PyMOL program (DeLano, W. L. The PyMOL Molecular Graphics System. (Palo Alto, 2002)). The core interface amino acids in the PAC1 ECD include Asp59, Asn60, Ile61, Arg116, Asn117, Thr119, Asp121, Gly122, Trp123, Ser124, Glu125, Pro126, Phe127, Pro128, His129, Tyr130, Phe131, Asp132, and Gly135 with the amino acid position numbers relative to SEQ ID NO: 1.

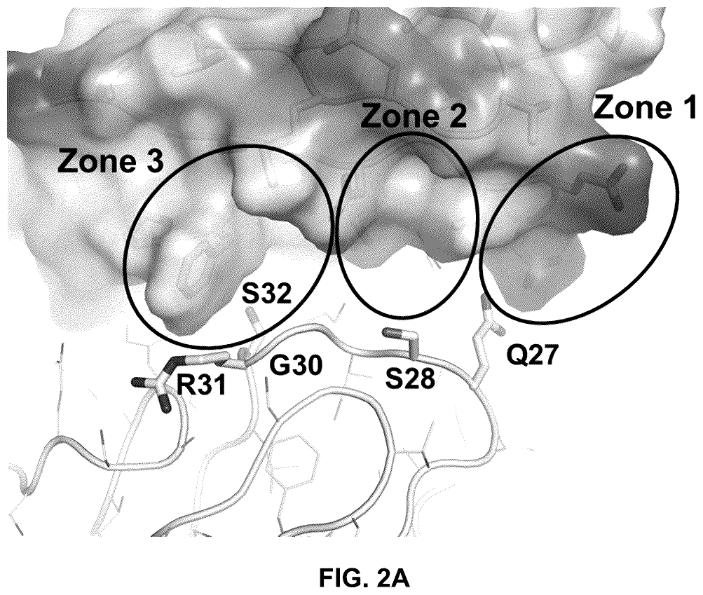

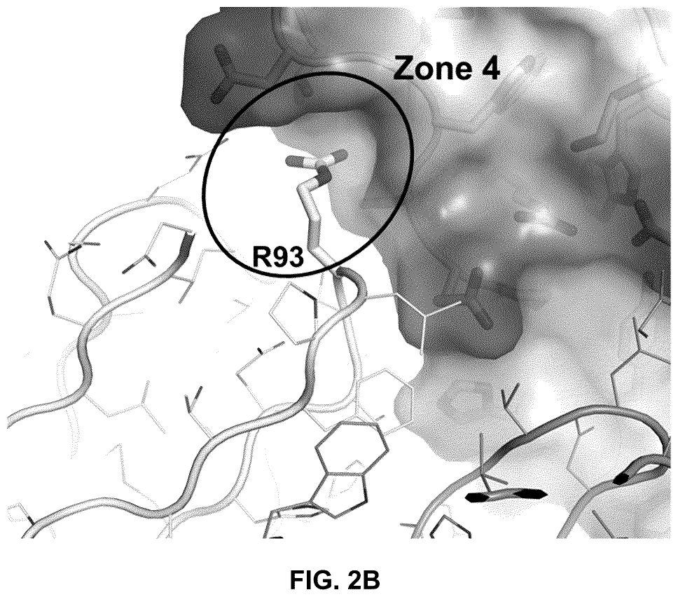

The interface between the 29G4v9 Fab and the PAC1 ECD in the crystal structure was analyzed to identify regions where the interactions between the two molecules were sub-optimal. Based on the structural analysis, mutations of the amino acids in the light and heavy chain variable regions were designed to enhance the interaction in these regions between the Fab and the PAC1 ECD to improve binding affinity and/or inhibitory potency of the antibody. In particular, analysis of the interaction between the Fab light chain and PAC1 ECD revealed four regions where mutations in the Fab light chain may possibly improve interactions with PAC1. In zone 1, a region containing PAC1 ECD amino acids Glu120 and Asp121, mutation of Gln27 in the light chain CDR1 (SEQ ID NO: 3) to lysine, tyrosine, or arginine was proposed to provide better charge complementarity or hydrogen bonding potential with the Glu120 and Asp121 PAC1 amino acids (FIG. 2A). Zone 2 is a region with positive electrostatic potential and mutation of Ser28 in the light chain CDR1 (SEQ ID NO: 3) to glutamate was proposed to provide better charge complementarity (FIG. 2A). Zone 3 is a hydrophobic region containing PAC1 ECD Phe127 residue and has hydrogen bonding potential. Gly30, Arg31, and Ser32 in the light chain CDR1 lie somewhat distant from zone 3 (FIG. 2A). Thus, multiple mutations were proposed at these three sites as summarized in Table 6 below to improve the hydrophobic or hydrogen bonding interactions between these three residues and this zone of the PAC1 ECD. Arg93 in the light chain CDR3 sits in a pocket of PAC1 residues with negative electrostatic potential (zone 4; FIG. 2B). However, due to the geometry, Arg93 does not form any direct hydrogen bonds with PAC1 residues. Mutations of Arg93 in the light chain CDR3 to glutamine, lysine, histidine, or asparagine were proposed to provide alternate charge complementarity or hydrogen bond potential with residues in the PAC1 ECD (FIG. 2B).

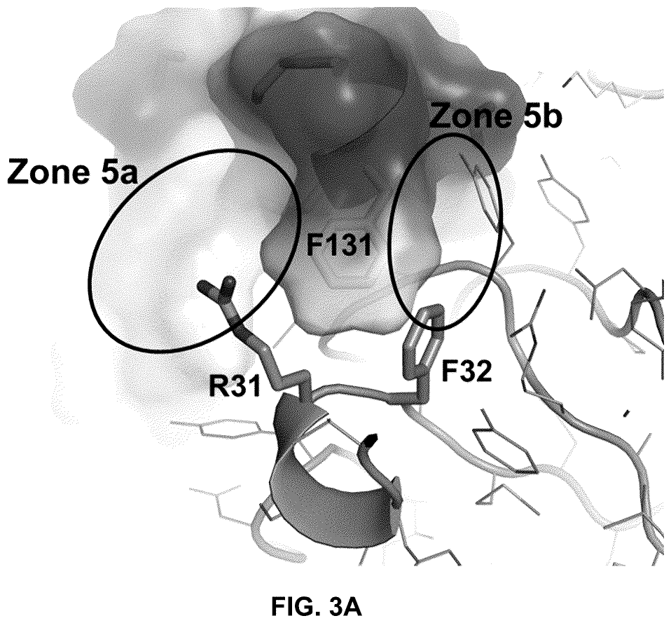

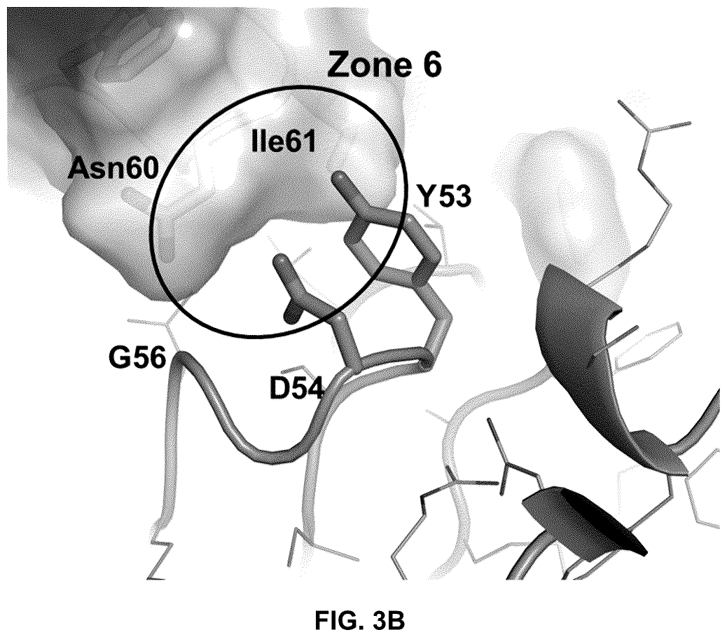

Analysis of the interaction between the 29G4v9 Fab heavy chain and PAC1 ECD revealed three main regions where mutations in the Fab heavy chain may improve interactions with the PAC1 ECD. As shown in FIG. 3A, zone 5 encompasses PAC1 amino acid Phe131, and can be divided into two sub-zones that lie on either side of Phe131. Arg31 and Phe32 in the heavy chain CDR1 lie in these sub-zones and mutations at these sites were proposed to improve the hydrophobic interactions with PAC1 Phe131 residue or provide alternate charge complementarity interactions. See Table 6 for list of mutations. In zone 6, which includes PAC1 ECD residues Asn60 and Ile61 (relative to SEQ ID NO: 1), mutation of heavy chain CDR2 residues Tyr53, Asp54 and Gly56 were proposed to improve hydrophobic interactions or provide alternate hydrogen bonding with Asn60 and Ile61 PAC1 residues (FIG. 3B). In zone 7, a region with hydrophobic residues and some negative electrostatic potential, mutations of heavy chain CDR3 residues Val102, Leu103 and Thr104 as set forth in Table 6 were proposed to improve hydrophobic interactions or provide alternate hydrogen bonding interactions (FIG. 3C).

A summary of the specific mutations proposed to improve the interaction between the anti-PAC1 antibody and the human PAC1 receptor based on the analysis of the crystal structure of the Fab/PAC1 ECD complex is provided in Table 6 below.

TABLE 6
Summary of Structure-Based Mutations in PAC1 Antibody
Variable Regions

| | |
|---|---|
| Amino Acid | |
| Position¹ | Mutations |

Light Chain

| | |
|---|---|
| Gln27 | Lys, Tyr, Arg |
| Ser28 | Glu |
| Gly30 | Leu, Val, Ile, Thr, Tyr, Phe, Met, Ala, His, |
| | Asn, Gln, Glu, Asp, Trp, Ser |
| Arg31 | Phe, Tyr, Leu, Ser, Thr, Gln, Asn |
| Ser32 | Leu, Thr, Ala, Met, Lys, Gln |
| Arg93 | Gln, Lys, His, Asn |

Heavy Chain

| | |
|---|---|
| Arg31 | Leu, Tyr, Met, Ile, Lys |
| Phe32 | Tyr, Lys, Gln |
| Tyr53 | Gln, Leu, Ser |
| Asp54 | Tyr, Gln, Asn |
| Gly56 | Ser, Thr |
| Val102 | Phe, Tyr, Trp, Leu, Thr, Ile, Met |
| Leu103 | Asn, Gln, Phe, Met, Ser, Thr |
| Thr104 | Ser |

¹Amino acid positions for the light chain are relative to SEQ ID NO: 3 and amino acid positions for the heavy chain are relative to SEQ ID NO: 2.

In addition to the mutations designed by analysis of the interacting amino acids between the anti-PAC1 Fab and the human PAC1 ECD as described above, an in silico affinity maturation analysis using the crystal structure was also conducted to identify additional mutations to improve the binding affinity and/or inhibitory potency of the anti-PAC1 antibody. Amino acid residues in the 29G4v9 Fab involved in binding to the human PAC1 ECD were identified by visual inspection of the crystal structure of the complex described above. These interface residues of the antibody were selected for virtual mutation to all other amino acids except for cysteine. The impact of the mutation to the antibody/PAC1 ECD binding interaction was assessed by calculating the change in binding free energy (ΔΔG_{binding}) upon mutation of a particular residue using Discovery Studio molecular modeling software from Biovia. A negative value of ΔΔG_{binding} indicates that the mutation results in a stronger binding to the PAC1 ECD compared the parental molecule. These calculations identified approximately 65 mutations that led to negative value of ΔΔG_{binding}. These 65 mutations were further narrowed down to 50 (18 in the light chain and 32 in the heavy chain) based on the visual inspection and analysis of the “modeled” structure of these variants with the PAC1 ECD. These 50 mutations predicted to increase the binding affinity of the antibody for PAC1 based on the binding free energy calculations are summarized in Table 7 below.

TABLE 7
Summary of Mutations in PAC1 Antibody
Variable Regions Predicted from In Silico Analysis

| | |
|---|---|
| Amino Acid | |
| Position¹ | Mutations |

Light Chain

| | |
|---|---|
| Gln27 | Lys, Tyr, Arg |
| Gly30 | Arg |
| Ser32 | Arg, Asn, His, Leu, Val |
| Ser91 | Lys, Thr |
| Ser92 | Gln, Ile |
| Leu94 | Arg, His, Tyr |
| Phe96 | Arg, Lys |

Heavy Chain

| | |
|---|---|
| Phe32 | His |
| Ala33 | Ser |
| Ser52 | Ile, Gln, Met |
| Asp54 | Tyr, Gln, Asn, Ile, Leu |
| Gly55 | Ala |
| Gly56 | Arg, Asn, His, Tyr |
| Asn57 | Arg, His, Lys, Tyr |
| Lys58 | Glu |
| Glu62 | Arg |
| Gly99 | Ala |
| Tyr100 | His |
| Val102 | Ile |
| Leu103 | Arg |
| Thr104 | Ser, His |
| Gly105 | Ser, Lys |
| Tyr106 | Arg, Lys |
| Asp108 | Arg |

¹Amino acid positions for the light chain are relative to SEQ ID NO: 3 and amino acid positions for the heavy chain are relative to SEQ ID NO: 2.

The in silico approach resulted in the identification of additional mutations, both in terms of different amino acids as well as different positions within the light and/or heavy chain, as compared with the structure-based analysis.

The mutations described in this example were incorporated into an anti-PAC1 antibody by recombinant production and tested for the ability to inhibit PACAP-induced activation of human PAC1 in an in vitro cell-based assay as described in Example 3 herein.

### Example 2. Yeast Display Affinity Maturation of 29G4v10 Human PAC1 Antibody

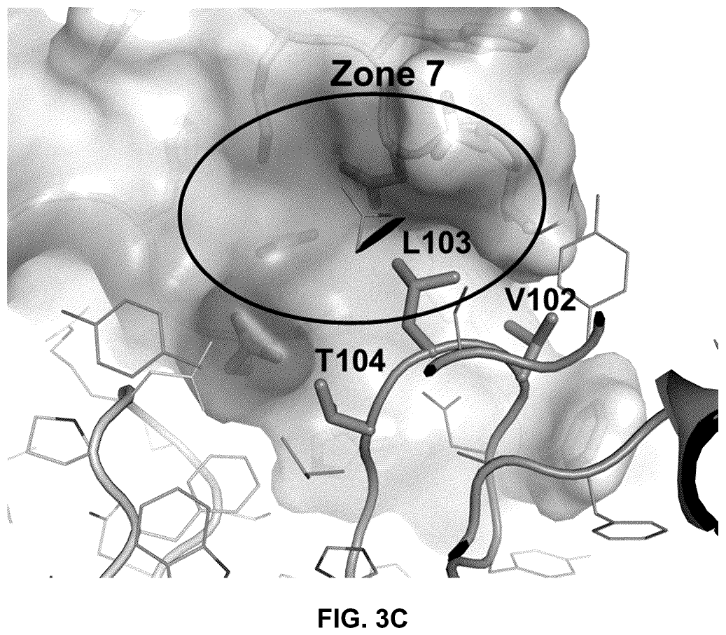

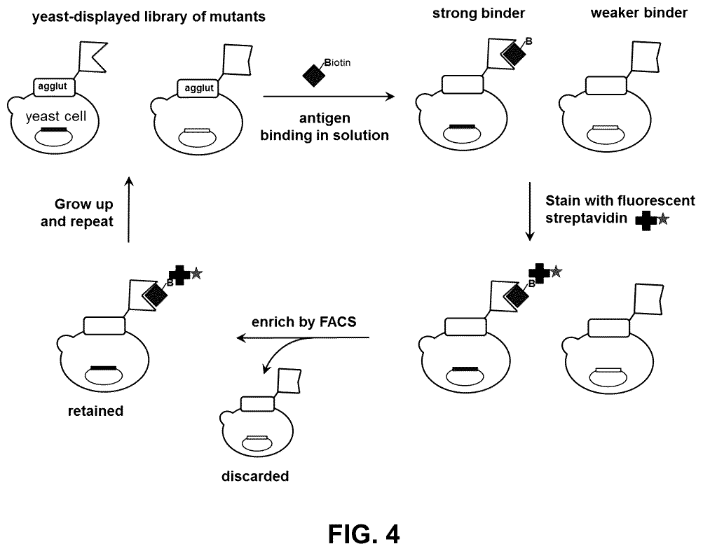

One powerful approach for affinity maturation involves constructing yeast-displayed libraries of antibody Fab mutants and selecting for improved binders through fluorescence-assisted cell sorting (FACS)(FIG. 4). To generate anti-PAC1 antibodies with improved inhibitory potency, the 29G4v10 antibody (VH region of SEQ ID NO: 191; VL region of SEQ ID NO: 52) was affinity matured by FACS of yeast-displayed Fab libraries. The library designs were guided by the solved co-crystal structure described in Example 1 of the complex between human PAC1 ECD and 29G4v9 Fab, the CDRs of which are nearly identical to those of 29G4v10. Structural analysis initially identified six and seven CDR positions within the light chain (LC) and heavy chain (HC) for mutagenesis, respectively, and suggested strategies for affinity enhancement. Point mutants were designed to improve hydrophobic interactions and charge complementarity, and to provide alternate hydrogen-bond interactions across the interface. See Example 1. These point mutants initiated an engineering approach that involved iterative rounds of production, characterization, and rational combination of beneficial mutations. The yeast library designs sought to complement the linear approach described in Example 1 by more comprehensively exploring mutation combinations among the 13 positions and augmenting the diversification strategy at each chosen position.

By making separate LC and HC libraries, the theoretical diversities were kept to a manageable size of <10⁷. The LC library was designed to utilize MIX19 saturation mutagenesis at five of the six chosen positions (G1n27, Gly30, Arg31, Ser32, and Arg93 of SEQ ID NO: 52). MIX19 represents a trimer phosphoramidite (codon) mixture encoding all amino acids except for cysteine. At the remaining light chain position (Ser28 of SEQ ID NO: 52), mutation to serine, glutamate, alanine or a stop codon was employed. To explore all seven HC positions (Arg31, Phe32, Tyr53, Gly56, Val102, Leu103, and Thr104 of SEQ ID NO: 191) in one library, we designed a custom mixture of nine codons (MIX9) for diversification at five of the seven heavy chain CDR positions (Arg31, Phe32, Tyr53, Val102, and Leu103 of SEQ ID NO: 191). Custom MIX9 comprised hydrophobic amino acids, basic amino acids, and hydrogen bond donors and acceptors (e.g. F, L, Y, M, Q, H, K, R, and S). Importantly, the custom MIX9 excluded cysteine, asparagine, and tryptophan to minimize introduction of sequence liabilities that could pose downstream manufacturability risks. Mutation to glycine, alanine, serine, and threonine was performed for the remaining two heavy chain positions (Gly56 and Thr104 of SEQ ID NO: 191). In summary, for this first campaign a mutHC library with a theoretical diversity of 8.5×10⁶ and a mutLC library with a theoretical diversity of 9.9×10⁶ were designed, and the constructed libraries oversampled the theoretical diversities by >9-fold.

The two constructed mutHC and mutLC Fab libraries were enriched for binding to the human PAC1 ECD using FACS, increasing the stringency with each successive round by lowering the concentration of ECD used for binding (Round 1: 30 nM PAC1 ECD; Round 2: 0.67 nM PAC1 ECD; and Round 3: 0.2 nM PAC1 ECD). An identically treated 29G4v10 Fab-yeast sample was routinely used to gate specifically for yeast clones exhibiting improved binding relative to the parental Fab. A normalized binding/display ratio for each clone was calculated by dividing the median fluorescence binding signal for each clone by the median fluorescence display signal for each clone. By round 3, improved binders were enriched from the mutHC library, but not the mutLC library. An abbreviated screen of −200 yeast clones from the mutHC Round 3 pool yielded 11 modestly improved binders (Table 8). The improved affinity variants were produced recombinantly and evaluated for in vitro functional activity as described in Example 3. Fortuitously, the aspartate isomerization site within the 29G4v10 parental Fab, a potential liability for manufacturability, was remediated in all of these 11 unique mutants.

TABLE 8
Top Improved Binders from MutHC Library Screen

| | | | | Yeast Binding Screen | |
|---|---|---|---|---|---|
| | Substitutions with respect | | | Data at 0.2 nM PAC1 | |
| | to 29G4v10 VH sequence | | | Normalized | |
| | (SEQ ID NO: 191)¹ | | | Binding Signal/ | Mutant vs. |
| Variant | HC | HC | HC | Display Signal | Wild-Type |
| Ab ID | CDR1 | CDR2 | CDR3 | (B/D) | B/D Ratio |
| iPS: 421855 | R31K; | Y53F; | V102L | 0.40 | 1.86 |
| | F32Y | D54K; | | | |
| | | G56S | | | |
| iPS: 421861 | F32Y | D54Q | V102L | 0.38 | 1.80 |
| iPS: 421867 | R31H | Y53F; | V102M | 0.35 | 1.61 |
| | | D54S; | | | |
| | | G56S | | | |
| iPS: 421873 | R31K | D54R | V102L | 0.42 | 1.95 |
| iPS: 421879 | F32Y | D54S; | V102L; | 0.33 | 1.56 |
| | | G56A | T104S | | |
| iPS: 421885 | R31H; | Y53F; | V102L | 0.45 | 2.09 |
| | F32Y | D54K; | | | |
| | | G56A | | | |
| iPS: 421891 | R31H; | D54R; | V102L | 0.49 | 2.27 |
| | F32Y | G56A | | | |
| iPS: 421897 | R31H; | Y53F; | V102F | 0.39 | 1.83 |
| | F32Y | D54Y; | | | |
| | | G56S | | | |
| iPS: 421903 | R31H | Y53F; | V102F | 0.44 | 2.07 |
| | | D54F | | | |
| iPS: 421909 | R31H; | Y53F; | V102F | 0.43 | 2.01 |
| | F32Y | D54M; | | | |
| | | G56T | | | |
| iPS: 421915 | R31Y | Y53H; | V102L; | 0.35 | 1.63 |
| | | D54R; | T104S | | |
| | | G56T | | | |
| 29G4v10 | — | — | — | 0.21 | 1.00 |
| Wild-Type | | | | | |

¹There were no changes in the sequence of the light chain relative to the 29G4v10 antibody for any of these variants.

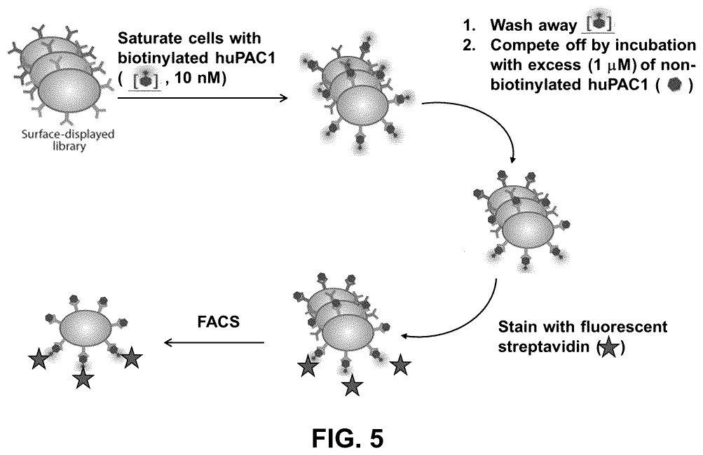

To generate further affinity improvements, a chain-shuffled library that combined enriched mutations from the mutHC and mutLC sorted pools was also constructed. To improve discrimination among the top binding clones, a binding off-rate driven selection and screening strategy was implemented (FIG. 5). Yeast cells displaying the mutant Fabs were first saturated with biotinylated human PAC1 ECD and washed extensively before a prolonged incubation in buffer containing a large excess of unlabeled human PAC1 ECD (up to 24 hours). The incubation with unlabeled human PAC1 ECD, which occurred at temperatures ranging from 25° C. to 37° C., rendered dissociation events from the cells irreversible. Cells retaining the most binding to biotinylated PAC1, thereby exhibiting the slowest off-rate, were isolated by FACS after staining with a fluorescent streptavidin conjugate (FIG. 5). Yeast clones displaying Fabs exhibiting the slowest off-rates were the most highly fluorescent.

In the off-rate driven sort of the chain-shuffled library, an identically treated 29G4v10 Fab-yeast sample was employed to specifically isolate cells with greater biotinylated PAC1 binding after overnight competition with unlabeled PAC1. From two pools collected under different gating stringencies, −600 individual yeast clones were screened using the binding off-rate assay and over 190 clones with higher remaining PAC1 binding than the parental 29G4v10 Fab were identified. Specificity of binding of these promising clones was evaluated by confirming there was no binding of the clones to the ECDs of unrelated receptors (programmed cell death protein 1 (PD1) and gastric inhibitory polypeptide receptor (GIPR)). Mutants containing additional cysteine anomalies, N-linked glycosylation, aspartate isomerization, asparagine deamidation, and tryptophan oxidation sites relative to the starting 29G4v10 sequence were also removed during the screen. The top 30 binding mutants are shown in Table 9 with sequence changes in the CDRs from parental 29G4v10 antibody and the percentage of human PAC1 ECD binding following the off-rate assay. Higher percentage association of PAC1 ECD binding indicates that the mutant Fabs have a slower off-rate.

TABLE 9
Top Improved Binders from Off-Rate Driven Screen of Chain-Shuffled Library

| | | | | | | | Secondary Screen: off-rate | |
|---|---|---|---|---|---|---|---|---|
| | | | | | | | competition at 30° C. | |
| | Substitutions with respect | | | Substitutions with respect | | | | Normalized |
| | to 29G4v10 VH sequence | | | to 29G4v10 VL sequence | | | | PAC1 ECD |
| | (SEQ ID NO: 191) | | | (SEQ ID NO: 52) | | | | binding remaining |
| | HC | HC | HC | LC | LC | LC | % association | (mutant vs. Q27K |
| Clone ID | CDR1 | CDR2 | CDR3 | CDR1 | CDR2 | CDR3 | after off-rate | LC variant) |
| 2_C11 | R31Y; | D54S; | V102L | Q27K; | — | — | 77% | 3.42 |
| | F32Y | G56S | | R31Q | | | | |
| 1_D07 | R31F; | Y53S; | V102L | Q27K; | — | — | 56% | 2.66 |
| | F32Y | D54F; | | R31Q | | | | |
| | | G56S | | | | | | |
| 2_B10 | R31H; | D54Q; | V102L | Q27R; | — | R93F | 54% | 2.79 |
| | F32Y | G56S | | R31L; | | | | |
| | | | | S32A | | | | |
| 2_G07 | R31Y; | Y53H; | V102P | Q27K; | — | — | 57% | 2.41 |
| | F32Y | D54Y; | | R31Q | | | | |
| | | G56T | | | | | | |
| 1_H04 | R31Y | Y53H; | V102L; | Q27K; | — | — | 52% | 2.37 |
| | | D54S; | T104S | R31Q | | | | |
| | | G56A | | | | | | |
| 1_F03 | — | — | — | Q27K; | — | — | 47% | 3.23 |
| | | | | R31W | | | | |
| 2_E08 | — | — | — | Q27K; | — | R93M | 50% | 2.29 |
| | | | | R31W | | | | |
| 1_F01 | R31H | D54S | V102L | Q27R; | — | R93M | 50% | 2.21 |
| | | | | R31L; | | | | |
| | | | | S32A | | | | |
| 2_F10 | R31H; | D54S | V102L; | Q27K; | — | — | 46% | 2.54 |
| | F32Y | | T104S | G30W; | | | | |
| | | | | R31K | | | | |
| 1_B08 | R31Y; | Y53S; | V102L | Q27K; | — | R93M | 50% | 2.18 |
| | F32Y | D54R; | | S28E; | | | | |
| | | G56S | | G30M; | | | | |
| | | | | R31S; | | | | |
| | | | | S32L; | | | | |
| | | | | L33H; | | | | |
| | | | | ΔH34¹ | | | | |
| 1_A11 | — | — | — | Q27R; | — | R93Y | 43% | 2.48 |
| | | | | S28A; | | | | |
| | | | | G30S; | | | | |
| | | | | R31N; | | | | |
| | | | | S32L; | | | | |
| | | | | L33H; | | | | |
| | | | | ΔH34¹ | | | | |
| 2_A10 | R31Y; | Y53S; | — | Q27K; | — | R93M | 55% | 1.70 |
| | F32Y | D54M; | | S28A; | | | | |
| | | G56T | | G30W; | | | | |
| | | | | R31H; | | | | |
| | | | | S32N | | | | |
| 2_G10 | R31M; | D54S; | V102L | Q27K; | — | R93M | 49% | 2.07 |
| | F32Y | G56S | | R31Q | | | | |
| 2_E10 | R31K; | D54M; | V102L | Q27K; | — | — | 68% | 1.53 |
| | F32Y | G56A | | R31W | | | | |
| 2_C05 | R31Y; | Y53S; | V102L | Q27K; | — | R93M | 48% | 1.90 |
| | F32Y | D54R; | | R31Q | | | | |
| | | G56S | | | | | | |
| 1_A09 | R31K; | D54S; | V102L | Q27K; | — | — | 36% | 2.38 |
| | F32Y | G56A | | R31Q | | | | |
| 2_C02 | R31K; | Y53F; | V102L; | Q27K; | — | R93M | 41% | 2.14 |
| | F32Y | D54S; | L103M | R31W | | | | |
| | | G56S | | | | | | |
| 1_H06 | F32Y | D54S; | V102L; | Q27K; | — | R93F | 38% | 2.16 |
| | | G56A | T104S | S28A; | | | | |
| | | | | R31F | | | | |
| 2_D01 | — | — | V102L | Q27K; | — | R93L | 34% | 2.10 |
| | | | | R31W | | | | |
| 2_B11 | R31Y; | Y53H; | V102L; | Q27R; | — | R93M | 45% | 1.53 |
| | F32Y | D54S | T104S | R31M | | | | |
| 2_F05 | R31H; | D54S | V102L; | Q27R; | — | R931 | 51% | 1.54 |
| | F32Y | | T104S | S28A; | | | | |
| | | | | G30F; | | | | |
| | | | | R31G; | | | | |
| | | | S32N | | | | | |
| 1_F10 | R31Y | Y53H; | V102L; | Q27K; | — | R93M | 39% | 1.96 |
| | | D54S; | T104S | S28A; | | | | |
| | | G56A | | G30W; | | | | |
| | | | | R31H; | | | | |
| | | | | S32N | | | | |
| 1_D05 | R31Y; | Y53S; | V102L | Q27H; | — | — | 45% | 1.82 |
| | F32Y | D54R; | | S28A; | | | | |
| | | G56S | | G30Y; | | | | |
| | | | | R31H; | | | | |
| | | | | S32N | | | | |
| 1_E01 | R31F | Y53S; | V102L; | Q27K; | — | — | 50% | 1.39 |
| | | D54H; | L103M; | R31W | | | | |
| | | G56T | T104S | | | | | |
| 2_H10 | — | — | — | Q27R; | — | R93M | 53% | 1.09 |
| | | | | S28A; | | | | |
| | | | | G30F; | | | | |
| | | | | R31G; | | | | |
| | | | | S32N | | | | |
| 2_B08 | — | D54S; | V102L | Q27K; | — | R93M | 43% | 1.91 |
| | | G56S | | S28A; | | | | |
| | | | | G30W; | | | | |
| | | | | R31H; | | | | |
| | | | | S32N | | | | |
| 1_A10 | F32Y | Y53F; | V102L | Q27K; | — | R93L | 49% | 1.65 |
| | | D54Q; | | R31W | | | | |
| | | G56S | | | | | | |
| 1_B11 | R31M | Y53H; | V102L | Q27K; | — | — | 44% | 1.65 |
| | | D54R; | | R31Q | | | | |
| | | G56T | | | | | | |
| 1_D04 | R31H; | D54R; | V102L | Q27K; | — | — | 37% | 2.12 |
| | F32Y | G56A | | R31Q | | | | |
| 1_C09 | R31H; | Y53F; | — | Q27K; | — | — | 39% | 1.80 |
| | F32Y | D54H | | R31Q | | | | |
| 29G4v10 | — | — | — | — | — | — | 25% | 0.70 |
| Wild-Type | | | | | | | | |
| VL Q27K | — | — | — | Q27K | — | — | 22% | 1.00 |
| variant | | | | | | | | |

¹The histidine residue at position 34 relative to the 29G4v10 light chain variable region sequence (SEQ ID NO: 52) is deleted in these mutants.

A set of second-generation libraries was designed to generate further affinity improvements. The first generation libraries described above focused diversification at a subset of the 29G4v10 parental antibody CDR positions that were within 4.5 Å of the PAC1 ECD in the crystal structure (Example 1). For the second generation libraries, mutagenesis in regions that were largely untouched in the first generation libraries, such as CDR2 and CDR3 of the light chain, were explored. For several positions already explored in the first generation libraries, sequencing trends were used to diversify to the limited set of neutral/beneficial mutations that had emerged after three rounds of sorting. Finally, some buried framework residues that could influence CDR conformations were targeted for limited diversification, with the mutation strategy favoring amino acids frequently found at the same position in other human VH3 and VK3 germlines.

In total, four second generation Fab libraries were designed to target 24 heavy chain CDR residues (amino acids 27, 29, 31-34, 49, 52-57, 70, 72, 77, 79, 98, 100-104, and 106 of SEQ ID NO: 191) and 17 light chain CDR residues (amino acids 27, 28, 30-32, 34, 46, 49-54, 92-94, and 96 of SEQ ID NO: 52) for diversification. The restricted diversity at many targeted positions enabled interrogation of more CDR positions within one library and co-optimization of HCDR1 with HCDR3 and LCDR1 with LCDR3. The four libraries that were constructed provided 8-24× coverage of the theoretical diversities ranging from 7×10⁶ to 1×10⁷.

The four constructed Fab libraries were enriched for binding to the human PAC1 ECD using FACS, as described above. By Round 3, only the mutHCDR2 and mutLCDR1-LCDR3 libraries yielded pools with equivalent or superior binding to parental 29G4v10 antibody. Therefore, these two pools were carried forward to make a mutH2/mutL1L3 chain-shuffled library for further affinity improvements. To enrich for the highest affinity mutants, 2B10, a top-performing yeast clone from the first generation libraries (Table 9), was used to set more stringent sort gates. Following an overnight off-rate competition with unlabeled PAC1 at 30° C. or 37° C., the yeast clones with significantly reduced off-rates compared to the 2B10 clone could be sorted out. A limited screen of −200 clones revealed that −100 had slower off-rates than 2B10, but most of the promising binders contained a potential asparagine deamidation liability within HCDR2. Non-specific binding to the ECDs of PD1 or GIPR was minimal for all clones screened. Applying stringent binding and sequence filters yielded ten top mutants with significantly improved PAC1 binding than the 2B10 clone (>2× higher percent association after 37° C. overnight competition) and without any potential sequence liabilities (Table 10). Sequence changes from parental 29G4v10 antibody for these top ten mutants and the percentage of human PAC1 ECD binding following the off-rate assay at 30° C. and 37° C. are shown in Table 10. Percentage association was calculated as the normalized binding after off rate assay divided by the normalized binding without the off rate assay. Higher percentage association of PAC1 ECD binding indicates that the mutant Fabs have a slower off-rate.

TABLE 10
Top Improved Binders from Off-Rate Screen of Second Generation Chain-Shuffled Library

| | | | | | | Substitutions with respect | | | | |
|---|---|---|---|---|---|---|---|---|---|---|
| | Substitutions with respect to 29G4v10 VH | | | | | to 29G4v10 VL sequence | | | | |
| | sequence (SEQ ID NO: 191) | | | | | (SEQ ID NO: 52) | | | % association after | |
| | HC | HC | HC | HC | HC | LC | LC | LC | off-rate screen | |
| Clone ID | CDR1 | FR2 | CDR2 | FR3 | CDR3 | CDR1 | CDR2 | CDR3 | 30° C. | 37° C. |
| 30_H10 | — | — | D54S; | I70V | — | Q27K; | — | — | 75.8% | 63.6% |
| | | | G56A; | | | R31W | | | | |
| | | | N57F | | | | | | | |
| 30_D05 | — | A49G | S52N; | I70V | — | Q27K; | — | — | 74.2% | 57.9% |
| | | | D54R; | | | R31W | | | | |
| | | | G56H; | | | | | | | |
| | | | N57G | | | | | | | |
| 30_F08 | — | A49G | S52T; | — | — | Q27K; | — | — | 67.1% | 54.6% |
| | | | D54T; | | | S28A; | | | | |
| | | | N57A | | | R31W | | | | |
| 30_A05 | — | A49G | S52N; | — | — | Q27K; | — | — | 65.8% | 50.5% |
| | | | D54F; | | | R31W | | | | |
| | | | G56D; | | | | | | | |
| | | | N57A | | | | | | | |
| 30_E08 | — | A49G | S52N; | — | — | Q27K; | — | — | 60.9% | 46.5% |
| | | | Y53F; | | | R31W | | | | |
| | | | D54Q; | | | | | | | |
| | | | G56T; | | | | | | | |
| | | | N57T | | | | | | | |
| 37_E09 | — | A49G | D54S; | I70V | — | Q27K; | — | — | 73.4% | 49.2% |
| | | | G56D; | | | R31W | | | | |
| | | | N57L | | | | | | | |
| 37_F04 | — | A49G | Y53F; | I70L | — | Q27K; | — | — | 73.0% | 46.5% |
| | | | D54S; | | | R31W | | | | |
| | | | N57S | | | | | | | |
| 37_B06 | — | A49G | D54S; | I70M | — | Q27K; | — | — | 68.6% | 43.0% |
| | | | G56A; | | | R31W | | | | |
| | | | N57S | | | | | | | |
| 37_H05 | — | A49G | D54T; | I70M | — | Q27K; | — | — | 64.3% | 40.2% |
| | | | G56A; | | | R31W | | | | |
| | | | N57Q | | | | | | | |
| 37_A11 | — | A49G | D54T; | I70V | — | Q27K; | — | — | 64.9% | 36.6% |
| | | | G56Q; | | | R31Y | | | | |
| | | | N57F | | | | | | | |
| 2_B10 | R31H; | — | D54Q; | — | V102L | Q27R; | — | R93F | 53.0% | 21.5% |
| Control | F32Y | | G56S | | | R31L; | | | | |
| | | | | | | S32A | | | | |
| 29G4v10 | — | — | — | — | — | — | — | — | 10.4% | 8.5% |
| Wild-Type | | | | | | | | | | |

As shown in Table 10, all of these top ten binding mutants contained a Q27K mutation in CDRL1 and all but one contained a R31W mutation in CDRL1. All the mutants had mutations at amino acid positions D54 and N57 in CDRH2 and most also had a mutation at amino acid position G56 in CDRH2. Conservative mutations at amino acid positions 49 and 70 in the heavy chain framework 2 (FR2) and framework 3 (FR3) regions, respectively, were also observed in many of these ten mutants.

In summary, guided by the structure of the PAC1 ECD in complex with the 29G4v9 Fab, a closely related variant of 29G4v22 and 29G4v10 anti-PAC1 neutralizing antibodies, combinatorial libraries of 29G4v10 mutants were designed and sorted for improved binding to the PAC1 ECD. To isolate the most improved binders, enriched mutations were combined through construction of CDR-shuffled and/or chain-shuffled libraries for selection under more stringent conditions. Screens of individual yeast Fab clones after sorting yielded improved binders to human PAC1 ECD with significantly slower binding off-rates as compared to the 29G4v10 parental antibody and minimal non-specific binding. A subset of the improved affinity variants were produced recombinantly and evaluated for in vitro functional activity as described in Example 3.

### Example 3. In Vitro Functional Activity of Human PAC1 Antibody Variants

To evaluate the effect of the mutations in the heavy and light chain variable regions identified by the analysis of the co-crystal structure (Example 1) or the yeast display libraries (Example 2) on the inhibitory potency of the anti-PAC1 antibody, the variants were produced by recombinant expression methods as complete bivalent monoclonal antibodies and/or as monovalent Fab-Fc fusions (e.g. Fab region fused to dimeric IgG Fc region) and evaluated in a cell-based cAMP assay as described in more detail below. The light chains of the monoclonal antibodies and Fab fragments comprised the light chain variable region from the indicated antibody variant fused to a human kappa light chain constant region having the sequence of SEQ ID NO: 318 or SEQ ID NO: 319. The heavy chains of the monoclonal antibodies and Fab-Fc fusions comprised the heavy chain variable region from the indicated antibody variant fused to an aglycosylated, disulfide-stabilized human IgG1z constant region having the sequence of SEQ ID NO: 325.

PAC1 antibody variant sequences were generated by site directed mutagenesis (SDM) or by Golden Gate Assembly (GGA) in those cases where SDM was unsuccessful. Site directed mutagenesis utilized paired mutagenic primers that flanked the mutation site. Whole vector PCR reactions were carried out using double stranded plasmid DNA templates. The primers for all of the desired mutations in a particular clone were combined into a master primer mix, with one to several mutations being incorporated in an individual reaction. Following amplification, the template plasmid DNA was removed by digestion with DpnI, an endonuclease which preferentially cleaves methylated DNA. The SDM product was then transformed into competent cells for growth and screening by sequencing. The SDM reactions were performed using the QuikChange Lightning Multi Site-Directed Mutagenesis Kit (Agilent) following the manufacturer's directions.

Where SDM was unsuccessful, an alternative cloning strategy was utilized. Briefly, GGA relied upon Type II restriction enzymes and T4 DNA ligase to cut and seamlessly ligate together multiple DNA fragments. (Engler et al., PLOS One, Vol. 3(11): e3647, 2008). In this example, the multiple DNA fragments consisted of (i) a synthetic nucleic acid sequence (gBlock, Integrated DNA Technologies, Coralville, IA) encoding a Kozak consensus sequence, a signal peptide sequence and an antibody variable region sequence; (ii) an antibody constant domain fragment released from a Parts vector; and (iii) the expression vector backbone. The GGA reactions were composed of 10 ng of gBlock, 10 ng of the Part vector, 10 ng of the expression vector, 1 μl 10× Fast Digest Reaction Buffer+0.5 mM ATP (Thermo Fisher, Waltham, MA), 0.5 μl FastDigest Esp3I (Thermo Fisher, Waltham, MA), 1 μl T4 DNA Ligase (5 U/μl, Thermo Fisher, Waltham, MA) and water to 10 μl. The reactions were performed over 15 cycles consisting of a 2 minute digestion step at 37° C. and a 3 minute ligation step at 16° C. The 15 cycles were followed by a final 5 minute 37° C. digestion step and a 5 minute enzyme inactivation step at 80° C.

Following cloning, PAC1 antibody polypeptides of which the first 22 amino acids were the VK1 signal peptide (MDMRVPAQLLGLLLLWLRGARC; SEQ ID NO: 486) were generated by transiently transfecting 293 HEK cells with the corresponding cDNAs. Cells at 1.5×10⁶ cells/ml were transfected with 0.5 mg/L DNA (0.5 mg/L PAC1 in the pTT5 vector) or (0.1 mg/L PAC1 in pTT5 vector with 0.4 mg/L empty pTT5 vector) (Durocher et al., NRCC, Nucleic Acids. Res., Vol. 30: e9, 2002) with 4 ml PEI/mg DNA in F17 media (Thermo Fisher). Yeastolate and Glucose were added to cultures 1 hour after transfection, and cells were then grown in suspension using F17 expression medium supplemented with 0.1% Kolliphor, 6 mM L-Glutamine and 50 μg/ml Geneticin for 6 days after which the conditioned media was harvested for purification.

The PAC1 antibody variants were purified from the conditioned media using protein A affinity chromatography (Mab Select SuRe, GE Healthcare Life Sciences, Little Chalfont, Buckinghamshire, UK) followed by cation exchange chromatography (SP Sepharose High Performance columns (SP HP) (GE Healthcare Life Sciences). The protein concentration of each purified pool was determined by UV absorbance at 280 nm (A280) using a NanoDrop 2000 (Thermo Fisher Scientific, Rockford, Illinois, USA). The purified pools were dialyzed against 2 L of 10 mM sodium-acetate, 9% sucrose, pH 5.2 (A52Su) using 20 kDa MWCO Slide-A-Lyzer dialysis flasks (Thermo Fisher Scientific) for 2 hours at 4° C. with gentle stirring on a stir plate. The used dialysate was decanted away, a fresh 2 L of A52Su was added and dialysis proceeded overnight. After dialysis, the samples were concentrated using 30 kDa MWCO ultrafiltration concentrators (Thermo Fisher Scientific) centrifuged at 2,000×g in a swinging-bucket rotor until each sample was approximately 40 mg/mL based on A280. The final products were analyzed for main peak purity using Caliper LabChip GXII microcapillary electrophoresis (PerkinElmer, Waltham, Massachusetts, USA) and size exclusion chromatography using an ACQUITY UPLC Protein BEH SEC column, 200 Å, 4.6×300 mm (Waters Corporation, Milford, Massachusetts, USA). The endotoxin content was measured using an Endosafe-MCS (Charles River, Wilmington, Massachusetts, USA) and 0.05 EU/mL PTS cartridges (Charles River).

The functional activity of the purified monoclonal antibodies or Fab-Fc fusion proteins was assessed using a cell-based PAC1 receptor cAMP activity assay. Both PACAP38 and PACAP27 are agonists of the PAC1 receptor, activation of which results in an increase in intracellular cAMP. The assay employed a human neuroblastoma-derived cell line (SH-SY5Y; Biedler J L et al., Cancer Res., Vol. 38: 3751-3757, 1978) obtained from ATCC (ATCC Number CRL-2266; “CRL-2266 cells”). CRL-2266 cells express human PAC1 receptor endogenously (Monaghan et al., J Neurochem., Vol. 104(1): 74-88, 2008). In addition, a CHO cell line stably expressing the rat PAC1 receptor (GenBank Accession No. NM 133511.2) or the cynomolgus monkey PAC1 receptor (NCBI Reference Sequence XP 015303041.1) was used in place of the CRL2266 cells for assays to evaluate the species cross-reactivity of the anti-PAC1 antibodies or Fab-Fc fusions at the rat and cynomolgus monkey PAC1 receptors. The LANCE Ultra cAMP assay kit (PerkinElmer, Boston, MA) was used to measure cAMP concentration.

On the day of the assay, the frozen CRL-2266 cells were thawed at 37° C. and were washed once with assay buffer. 10 μL of cell suspension containing 2,000 cells was added into 96 half-area white plates. After adding 5 μL of the anti-PAC1 variant monoclonal antibody or Fab-Fc fusion protein (10 point dose response curve: concentration range from 1 μM to 0.5 fM), the mixture was incubated for 30 min at room temperature. Then, 5 μL of human PACAP38 (10 pM final concentration) was added as an agonist and the mixture was further incubated for 15 min at room temperature. After human PACAP38 stimulation, 20 μL of detection mix was added and incubated for 45 minutes at room temperature. The plates were read on EnVision instrument (PerkinElmer, Boston, MA) at emission wavelength 665 nm. Data were processed and analyzed by Prizm (GraphPad Software Inc.) to show POC (percent of control, in which control is defined as the activity of the agonist used in the assay) as a function of the tested antagonist concentration (e.g. anti-PAC1 variant antibody or Fab-Fc fusion protein), and were fitted with standard nonlinear regression curves to yield IC50 values. POC was calculated as follows:

$$POC = 1 \; 00 \times Em665 of \frac{\begin{matrix}Agonist response with antagonist - \\ cell response without agonist and antagonist\end{matrix}}{\begin{matrix}Agonist response without antagonist - \\ cell response without agonist and antagonist\end{matrix}}$$

Monovalent Fab-Fc fusion proteins were generated with the mutations summarized in Tables 6 and 7 and tested for functional activity in the cell-based cAMP assay described above. The mutant Fab-Fc fusion proteins were segregated into two separate groups: mutations that improve inhibitory potency against the human PAC1 receptor compared to the parent molecule and mutations characterized as neutral (Table 11). Mutations were characterized as neutral if the mutation had an average potency less than 1.5× weaker than that of parent molecule, and at least one potency measurement tighter than parent molecule in same run.

TABLE 11
In vitro inhibitory potency of single mutant
Fab-Fc fusion proteins

| | | |
|---|---|---|
| Fab-Fc Fusion Protein ID No. | Mutations¹ | IC50 (nM) |

Improved Potency Mutations

| | | |
|---|---|---|
| 29G4v10 Fab-Fc fusion parent | — | 10.37 |
| molecule | | |
| PL-45360 | LC Q27K | 4.52 |
| PL-45362 | LC Q27R | 6.32 |
| PL-45398 | HC D54N | 1.78 |
| PL-45399 | HC D54I | 2.48 |
| PL-45400 | HC D54L | 3.11 |
| PL-45405 | HC D54Q | 3.46 |
| PL-45397 | HC D54Y | 3.62 |
| PL-45406 | HC G56R | 2.81 |
| PL-45407 | HC G56N | 7.98 |
| PL-45408 | HC G56H | 9.46 |
| PL-45402 | HC G56S | 9.49 |
| PL-45415 | HC E62R | 4.27 |
| PL-45418 | HC V102I | 9.68 |

Neutral Mutations

| | | |
|---|---|---|
| 29G4v10 Fab-Fc fusion parent | — | 10.37 |
| molecule | | |
| PL-45370 | LC S91T | 15.19 |
| PL-45377 | LC L94R | 11.05 |
| PL-45386 | HC R31K | 12.12 |
| PL-45401 | HC G55A | 12.58 |
| PL-45410 | HC N57R | 11.63 |
| PL-45420 | HC T104H | 11.29 |
| PL-45420 | HC T104S | 13.36 |

¹Amino acid positions for the light chain (LC) are relative to SEQ ID NO: 52 and amino acid positions for the heavy chain (HC) are relative to SEQ ID NO: 191.

The mutations that provided increases in inhibitory potency against the human PAC1 receptor lie in three distinct regions of three dimensional space on the antibody surface. A second round of variant antibodies and Fab-Fc fusion proteins were generated by combining mutations in these three regions to potentially provide additional increases in potency. In addition, some of the neutral mutations were incorporated as they were likely to not have a significant effect on binding, but may provide mechanisms to modify antibody biophysical characteristics. The variable regions containing the desired mutations were incorporated into a bivalent monoclonal antibody having a human aglycosylated IgG1 Fc region and/or a monovalent Fab-Fc fusion protein. The aglycosylated human IgG1 antibody Fc region comprised the sequence of a human IgG1z Fc region with N297G, R292C, and V302C mutations according to EU numbering (SEQ ID NO: 325). The variant antibodies and Fab-Fc fusion proteins with combinations of mutations were evaluated for functional activity in the cell-based cAMP assay. The results are shown in Table 12 below.

TABLE 12
In vitro inhibitory potency of variant antibodies and Fab-Fc fusion proteins

| | | | | | | | | | | Fab-Fc fusion protein | | |
|---|---|---|---|---|---|---|---|---|---|---|---|---|
| | | | | | | | mAb functional activity | | | functional activity | | |
| | | | | | | | (bivalent target binding) | | | (monovalent target binding) | | |
| | | | | | | | | Fold- | | | Fold- | |
| | Substitutions with respect | | | Substitutions with respect | | | | increase | Fold- | | increase | Fold- |
| | to 29G4v10 VL sequence | | | to 29G4v10 VH sequence | | | | over | increase | | over | increase |
| | (SEQ ID NO: 52) | | | (SEQ ID NO: 191) | | | IC50 | parental | over | IC50 | parental | over |
| Variant Ab | LC | LC | LC | HC | HC | HC | (SD) | (on | parental | (SD) | (on | parental |
| ID. | CDR1 | CDR2 | CDR3 | CDR1 | CDR2 | CDR3 | in nM | plate)¹ | (avg)² | in nM | plate)¹ | (avg)² |
| iPS:420649 | Q27K | — | — | — | D54N | — | 0.18 | 6.4 | 4.3 | 0.71 | 10.5 | 12.9 |
| | | | | | | | (0.04) | | | (0.09) | | |
| iPS:420653 | Q27K | — | — | — | D54I | — | 0.18 | 6.4 | 4.3 | 0.99 | 8.1 | 9.2 |
| | | | | | | | (0.06) | | | (0.13) | | |
| iPS:420657 | Q27K | — | — | — | D54Q | — | 0.29 | 2.5 | 2.7 | 0.87 | 9.2 | 10.4 |
| | | | | | | | (0.05) | | | (0.23) | | |
| iPS:420661 | Q27K | — | — | — | D54Y | — | 0.21 | 3.4 | 3.7 | 1.83 | 4.4 | 5.0 |
| | | | | | | | (0.05) | | | (0.28) | | |
| iPS:420665 | Q27K | — | — | — | G56R | — | 0.44 | 1.7 | 1.8 | 1.94 | 4.1 | 4.7 |
| | | | | | | | (0.19) | | | (0.50) | | |
| iPS:420672 | Q27K | — | — | — | G56N | — | 1.04 | 0.7 | 0.7 | 3.17 | 2.5 | 2.9 |
| | | | | | | | (0.50) | | | (0.97) | | |
| iPS:420679 | Q27K | — | — | — | E62R | — | 0.47 | 1.6 | 1.7 | 1.34 | 6.0 | 6.8 |
| | | | | | | | (0.22) | | | (0.31) | | |
| iPS:420686 | Q27K | — | — | — | — | V102I | 0.78 | 0.9 | 1.0 | 1.92 | 4.2 | 4.7 |
| | | | | | | | (0.39) | | | (0.19) | | |
| iPS:420690 | — | — | — | — | D54N; | — | 0.16 | 4.4 | 4.8 | 1.86 | 3.6 | 4.9 |
| | | | | | G56R | | (0.02) | | | (0.17) | | |
| iPS:420697 | — | — | — | — | D54I; | — | 0.20 | 3.4 | 4.0 | 1.48 | 4.5 | 6.1 |
| | | | | | G56R | | (0.06) | | | (0.48) | | |
| iPS:420704 | — | — | — | — | D54Q; | — | 0.18 | 3.7 | 4.4 | 2.00 | 3.4 | 4.5 |
| | | | | | G56R | | (0.05) | | | (0.20) | | |
| iPS:420711 | — | — | — | — | D54Y; | — | 0.45 | 1.5 | 1.7 | 3.45 | 1.9 | 2.6 |
| | | | | | G56R | | (0.29) | | | (0.13) | | |
| iPS:420718 | — | — | — | — | D54N; | — | 0.60 | 1.1 | 1.3 | 3.33 | 2.0 | 2.7 |
| | | | | | G56N | | (0.21) | | | (0.63) | | |
| iPS:420725 | — | — | — | — | D54I; | — | 0.56 | 1.2 | 1.4 | 4.06 | 1.7 | 2.2 |
| | | | | | G56N | | (0.04) | | | (0.58) | | |
| iPS:420732 | — | — | — | — | D54Q; | — | 0.74 | 0.9 | 1.1 | 1.82 | 3.7 | 5.0 |
| | | | | | G56N | | (0.18) | | | (0.32) | | |
| iPS:420739 | — | — | — | — | D54Y; | — | 0.63 | 1.1 | 1.2 | 2.76 | 2.9 | 3.3 |
| | | | | | G56N | | (0.13) | | | (0.34) | | |
| iPS:420746 | — | — | — | — | D54N; | — | 0.18 | 4.2 | 4.4 | 0.81 | 9.8 | 11.3 |
| | | | | | E62R | | (0.04) | | | (0.13) | | |
| iPS:420753 | — | — | — | — | D54I; | — | 0.16 | 4.8 | 5.0 | 1.55 | 5.1 | 5.9 |
| | | | | | E62R | | (0.05) | | | (0.11) | | |
| iPS:420760 | — | — | — | — | D54Q; | — | 0.32 | 2.4 | 2.5 | 2.70 | 2.9 | 3.4 |
| | | | | | E62R | | (0.06) | | | (0.85) | | |
| iPS:420767 | — | — | — | — | D54Y; | — | ND | ND | ND | 1.49 | 5.3 | 6.1 |
| | | | | | E62R | | | | | (0.54) | | |
| iPS:420774 | — | — | — | — | D54N | V102I | 0.69 | 1.1 | 1.1 | 1.04 | 7.6 | 8.7 |
| | | | | | | | (0.30) | | | (0.20) | | |
| iPS:420781 | — | — | — | — | D54I | V102I | 0.40 | 1.9 | 1.9 | 1.81 | 4.4 | 5.0 |
| | | | | | | | (0.05) | | | (0.43) | | |
| iPS:420788 | — | — | — | — | D54Q | V102I | 0.46 | 1.6 | 1.7 | 1.59 | 6.0 | 5.7 |
| | | | | | | | (0.17) | | | (0.83) | | |
| iPS:420795 | — | — | — | — | D54Y | V102I | 0.50 | 1.5 | 1.5 | 4.25 | 2.3 | 2.1 |
| | | | | | | | (0.09) | | | (1.91) | | |
| iPS:420802 | — | — | — | — | G56R; | — | 0.17 | 3.9 | 4.6 | 1.51 | 6.4 | 6.0 |
| | | | | | E62R | | (0.05) | | | (0.01) | | |
| iPS:420809 | — | — | — | — | G56N; | — | 0.77 | 0.9 | 1.0 | 2.51 | 3.8 | 3.6 |
| | | | | | E62R | | (0.20) | | | (0.28) | | |
| iPS:420816 | — | — | — | — | G56R | V102I | 0.50 | 1.3 | 1.6 | 2.44 | 3.9 | 3.7 |
| | | | | | | | (0.07) | | | (0.43) | | |
| iPS:420823 | — | — | — | — | G56N | V102I | 1.41 | 0.5 | 0.6 | 2.30 | 4.2 | 3.9 |
| | | | | | | | (0.42) | | | (0.42) | | |
| iPS:420830 | — | — | — | — | E62R | V102I | 0.91 | 0.7 | 0.9 | 1.73 | 5.5 | 5.3 |
| | | | | | | | (0.64) | | | (0.16) | | |
| iPS:420837 | Q27K | — | — | — | D54N; | — | 0.21 | 3.2 | 3.7 | 0.67 | 12.4 | 13.5 |
| | | | | | G56R | | (0.10) | | | (0.18) | | |
| iPS:420841 | Q27K | — | — | — | D54I; | — | 0.14 | 6.1 | 5.6 | 0.69 | 12.0 | 13.1 |
| | | | | | G56R | | (0.00) | | | (0.10) | | |
| iPS:420845 | Q27K | — | — | — | D54Q; | — | 0.12 | 7.5 | 6.8 | 0.83 | 10.1 | 10.9 |
| | | | | | G56R | | (0.04) | | | (0.14) | | |
| iPS:420849 | Q27K | — | — | — | D54Y; | — | 0.22 | 4.0 | 3.6 | 1.73 | 4.8 | 5.2 |
| | | | | | G56R | | (0.04) | | | (0.74) | | |
| iPS:420853 | Q27K | — | — | — | D54N; | — | 0.39 | 2.2 | 2.0 | 1.93 | 4.3 | 4.7 |
| | | | | | G56N | | (0.06) | | | (0.73) | | |
| iPS:420857 | Q27K | — | — | — | D54I; | — | 0.47 | 1.8 | 1.7 | 1.94 | 4.3 | 4.7 |
| | | | | | G56N | | (0.13) | | | (0.27) | | |
| iPS:420861 | Q27K | — | — | — | D54Q; | — | 0.43 | 2.0 | 1.8 | 1.13 | 7.4 | 8.1 |
| | | | | | G56N | | (0.01) | | | (0.37) | | |
| iPS:420865 | Q27K | — | — | — | D54Y; | — | 0.39 | 2.2 | 2.0 | 3.23 | 4.1 | 2.8 |
| | | | | | G56N | | (0.03) | | | (1.03) | | |
| iPS:420869 | Q27K | — | — | — | D54N; | — | ND | ND | ND | 0.91 | 14.3 | 10.0 |
| | | | | | E62R | | | | | (0.06) | | |
| iPS:420873 | Q27K | — | — | — | D54I; | — | ND | ND | ND | 1.34 | 9.8 | 6.8 |
| | | | | | E62R | | | | | (0.40) | | |
| iPS:420877 | Q27K | — | — | — | D54Q; | — | ND | ND | ND | 1.64 | 8.0 | 5.5 |
| | | | | | E62R | | | | | (0.51) | | |
| iPS:420881 | Q27K | — | — | — | D54Y; | — | ND | ND | ND | 1.33 | 9.8 | 6.8 |
| | | | | | E62R | | | | | (0.27) | | |
| iPS:420885 | Q27K | — | — | — | D54N | V102I | 0.15 | 3.7 | 5.4 | 1.18 | 11.1 | 7.7 |
| | | | | | | | (0.01) | | | (0.04) | | |
| iPS:420889 | Q27K | — | — | — | D54I | V102I | 0.16 | 3.4 | 4.9 | 1.87 | 7.0 | 4.8 |
| | | | | | | | (0.01) | | | (0.39) | | |
| iPS:420893 | Q27K | — | — | — | D54Q | V102I | 0.30 | 1.8 | 2.6 | 1.58 | 8.2 | 5.8 |
| | | | | | | | (0.08) | | | (0.33) | | |
| iPS:420897 | Q27K | — | — | — | D54Y | V102I | 0.49 | 1.1 | 1.6 | 4.05 | 3.2 | 2.2 |
| | | | | | | | (0.18) | | | (0.38) | | |
| iPS:420901 | — | — | — | — | D54N; | — | ND | ND | ND | 2.06 | 6.3 | 4.4 |
| | | | | | G56R; | | | | | (0.61) | | |
| | | | | | E62R | | | | | | | |
| iPS:420908 | — | — | — | — | D54I; | — | ND | ND | ND | 1.46 | 8.9 | 6.2 |
| | | | | | G56R; | | | | | (1.11) | | |
| | | | | | E62R | | | | | | | |
| iPS:420915 | — | — | — | — | D54Q; | — | ND | ND | ND | 0.81 | 15.9 | 11.2 |
| | | | | | G56R; | | | | | (0.20) | | |
| | | | | | E62R | | | | | | | |
| iPS:420922 | — | — | — | — | D54Y; | — | ND | ND | ND | 1.01 | 12.9 | 9.0 |
| | | | | | G56R; | | | | | (0.21) | | |
| | | | | | E62R | | | | | | | |
| iPS:420929 | — | — | — | — | D54N; | — | 0.12 | 4.5 | 6.5 | 0.84 | 13.9 | 10.8 |
| | | | | | G56N; | | (0.00) | | | (0.27) | | |
| | | | | | E62R | | | | | | | |
| iPS:420936 | — | — | — | — | D54I; | — | 0.16 | 3.4 | 4.9 | 1.89 | 6.2 | 4.8 |
| | | | | | G56N; | | (0.01) | | | (0.54) | | |
| | | | | | E62R | | | | | | | |
| iPS:420943 | — | — | — | — | D54Q; | — | 0.17 | 3.8 | 4.7 | 3.62 | 3.2 | 2.5 |
| | | | | | G56N; | | (0.01) | | | (1.42) | | |
| | | | | | E62R | | | | | | | |
| iPS:420950 | — | — | — | — | D54Y; | — | 0.30 | 2.1 | 2.6 | 3.45 | 3.4 | 2.6 |
| | | | | | G56N; | | (0.06) | | | (1.38) | | |
| | | | | | E62R | | | | | | | |
| iPS:420957 | — | — | — | — | D54N; | V102I | 0.27 | 2.3 | 2.9 | 2.83 | 4.1 | 3.2 |
| | | | | | G56R | | (0.01) | | | (1.54) | | |
| iPS:420964 | — | — | — | — | D54I; | V102I | 0.37 | 1.7 | 2.1 | 2.86 | 4.1 | 3.2 |
| | | | | | G56R | | (0.09) | | | (1.29) | | |
| iPS:420971 | — | — | — | — | D54Q; | V102I | 0.22 | 2.8 | 3.5 | 3.52 | 3.3 | 2.6 |
| | | | | | G56R | | (0.00) | | | (1.74) | | |
| iPS:420978 | — | — | — | — | D54Y; | V102I | 0.28 | 2.3 | 2.8 | 5.24 | 2.7 | 1.7 |
| | | | | | G56R | | (0.01) | | | (1.26) | | |
| iPS:420985 | — | — | — | — | D54N; | V102I | ND | ND | ND | 2.86 | 4.9 | 3.2 |
| | | | | | G56N | | | | | (0.32) | | |
| iPS:420992 | — | — | — | — | D54I; | V102I | 0.38 | 1.6 | 2.0 | 6.18 | 2.3 | 1.5 |
| | | | | | G56N | | (0.07) | | | (0.84) | | |
| iPS:420999 | — | — | — | — | D54Q; | V102I | 0.63 | 1.0 | 1.2 | 11.60 | 1.2 | 0.8 |
| | | | | | G56N | | (0.18) | | | (1.89) | | |
| iPS:421006 | — | — | — | — | D54Y; | V102I | 1.10 | 0.6 | 0.7 | 13.82 | 1.0 | 0.7 |
| | | | | | G56N | | (0.17) | | | (1.13) | | |
| iPS:421013 | — | — | — | — | G56R; | V102I | 0.32 | 2.0 | 2.4 | 2.07 | 6.8 | 4.4 |
| | | | | | E62R | | (0.03) | | | (0.61) | | |
| iPS:421020 | — | — | — | — | G56N; | V102I | 0.54 | 1.2 | 1.5 | 2.65 | 5.3 | 3.4 |
| | | | | | E62R | | (0.01) | | | (0.53) | | |
| iPS:421027 | Q27K | — | — | — | D54N; | — | ND | ND | ND | 0.91 | 12.5 | 10.0 |
| | | | | | G56R; | | | | | (0.41) | | |
| | | | | | E62R | | | | | | | |
| iPS:421031 | Q27K | — | — | — | D54I; | — | ND | ND | ND | 0.82 | 13.8 | 11.0 |
| | | | | | G56R; | | | | | (0.31) | | |
| | | | | | E62R | | | | | | | |
| iPS:421035 | Q27K | — | — | — | D54Q; | — | ND | ND | ND | 1.22 | 9.3 | 7.4 |
| | | | | | G56R; | | | | | | (0.29) | |
| | | | | | E62R | | | | | | | |
| iPS:421039 | Q27K | — | — | — | D54Y; | — | ND | ND | ND | 2.98 | 3.8 | 3.1 |
| | | | | | G56R; | | | | | (2.17) | | |
| | | | | | E62R | | | | | | | |
| iPS:421043 | Q27K | — | — | — | D54N; | — | ND | ND | ND | 1.44 | 7.9 | 6.3 |
| | | | | | G56N; | | | | | (0.53) | | |
| | | | | | E62R | | | | | | | |
| iPS:421047 | Q27K | — | — | — | D54I; | — | 0.10 | 6.5 | 8.1 | 1.29 | 8.8 | 7.1 |
| | | | | | G56N; | | (0.00) | | | (0.59) | | |
| | | | | | E62R | | | | | | | |
| iPS:421051 | Q27K | — | — | — | D54Q; | — | 0.09 | 7.0 | 8.6 | 1.80 | 6.3 | 5.1 |
| | | | | | G56N; | | (0.01) | | | (0.99) | | |
| | | | | | E62R | | | | | | | |
| iPS:421055 | Q27K | — | — | — | D54Y; | — | 0.43 | 1.6 | 1.8 | 1.06 | 7.7 | 8.6 |
| | | | | | G56N; | | (0.04) | | | (0.18) | | |
| | | | | | E62R | | | | | | | |
| iPS:421059 | Q27K | — | — | — | D54N; | V102I | 0.10 | 6.8 | 7.8 | 1.01 | 8.1 | 9.0 |
| | | | | | G56R | | (0.04) | | | (0.07) | | |
| iPS:421063 | Q27K | — | — | — | D54I; | V102I | 0.23 | 3.0 | 3.4 | 1.12 | 7.3 | 8.1 |
| | | | | | G56R | | (0.01) | | | (0.12) | | |
| iPS:421067 | Q27K | — | — | — | D54Q; | V102I | 0.30 | 2.3 | 2.6 | 1.49 | 5.5 | 6.1 |
| | | | | | G56R | | (0.04) | | | (0.43) | | |
| iPS:421071 | Q27K | — | — | — | D54Y; | V102I | 0.30 | 2.3 | 2.6 | 1.18 | 6.9 | 7.7 |
| | | | | | G56R | | (0.14) | | | (0.05) | | |
| iPS:421075 | Q27K | — | — | — | D54N; | V102I | ND | ND | ND | 1.02 | 8.0 | 8.9 |
| | | | | | G56N | | | | | (0.21) | | |
| iPS:421079 | Q27K | — | — | — | D54I; | V102I | 0.30 | 2.2 | 2.6 | 1.21 | 5.9 | 7.5 |
| | | | | | G56N | | (0.07) | | | (0.04) | | |
| iPS:421083 | Q27K | — | — | — | D54Q; | V102I | 0.51 | 1.3 | 1.5 | 1.73 | 4.1 | 5.2 |
| | | | | | G56N | | (0.29) | | | (0.03) | | |
| iPS:421087 | Q27K | — | — | — | D54Y; | V102I | 0.33 | 2.3 | 2.4 | 2.38 | 3.0 | 3.8 |
| | | | | | G56N | | (0.05) | | | (0.63) | | |
| iPS:421091 | — | — | — | — | D54N; | V102I | 0.07 | 11.4 | 11.7 | 1.41 | 5.1 | 6.5 |
| | | | | | G56R; | | (0.00) | | | (0.26) | | |
| | | | | | E62R | | | | | | | |
| iPS:421098 | — | — | — | — | D54I; | V102I | 0.17 | 4.6 | 4.7 | 1.26 | 5.7 | 7.2 |
| | | | | | G56R; | | (0.01) | | | (0.14) | | |
| | | | | | E62R | | | | | | | |
| iPS:421105 | — | — | — | — | D54Q; | V102I | 0.19 | 4.0 | 4.1 | 1.12 | 6.4 | 8.1 |
| | | | | | G56R; | | (0.04) | | | (0.17) | | |
| | | | | | E62R | | | | | | | |
| iPS:421112 | — | — | — | — | D54Y; | V102I | 0.21 | 3.6 | 3.7 | 1.38 | 5.2 | 6.6 |
| | | | | | G56R; | | (0.01) | | | (0.33) | | |
| | | | | | E62R | | | | | | | |
| iPS:421119 | — | — | — | — | D54N; | V102I | 0.21 | 3.6 | 3.7 | 1.20 | 9.6 | 7.6 |
| | | | | | G56N; | | (0.00) | | | (0.17) | | |
| | | | | | E62R | | | | | | | |
| iPS:421126 | — | — | — | — | D54I; | V102I | 0.19 | 4.0 | 4.1 | 1.95 | 5.9 | 4.7 |
| | | | | | G56N; | | (0.01) | | | (0.14) | | |
| | | | | | E62R | | | | | | | |
| iPS:421133 | — | — | — | — | D54Q; | V102I | 0.29 | 3.1 | 2.7 | 4.46 | 2.6 | 2.0 |
| | | | | | G56N; | | (0.05) | | | (1.18) | | |
| | | | | | E62R | | | | | | | |
| iPS:421140 | — | — | — | — | D54Y; | V102I | 0.37 | 2.4 | 2.1 | 5.48 | 2.1 | 1.7 |
| | | | | | G56N; | | (0.04) | | | (1.06) | | |
| | | | | | E62R | | | | | | | |
| iPS:421147 | Q27K | — | — | — | D54Y; | V102I | 1.15 | 0.8 | 0.7 | 1.81 | 6.4 | 5.0 |
| | | | | | G56N; | | (0.80) | | | (0.47) | | |
| | | | | | E62R | | | | | | | |
| iPS:421151 | G30W | — | — | — | — | — | 6.66 | 0.1 | 0.1 | 12.50 | 0.9 | 0.7 |
| | | | | | | | (2.26) | | | (3.25) | | |
| iPS:391478 | S32N | — | — | — | — | — | 6.16 | 0.1 | 0.1 | 36.74 | 0.3 | 0.2 |
| | | | | | | | (3.39) | | | (9.14) | | |
| iPS:421157 | — | — | R93M | — | — | — | 8.04 | 0.1 | 0.1 | 6.60 | 2.0 | 1.4 |
| | | | | | | | (3.91) | | | (1.31) | | |
| iPS:421163 | — | — | — | R31H | — | — | 14.01 | 0.1 | 0.1 | 4.99 | 2.6 | 1.8 |
| | | | | | | | (8.21) | | | (0.69) | | |
| iPS:391578 | — | — | — | F32Y | — | — | 2.68 | 0.3 | 0.3 | 4.91 | 2.7 | 1.9 |
| | | | | | | | (1.22) | | | (0.37) | | |
| iPS:421170 | — | — | — | — | Y53F | — | 3.18 | 0.2 | 0.2 | 4.56 | 2.9 | 2.0 |
| | | | | | | | (0.65) | | | (0.87) | | |
| iPS:421176 | — | — | — | — | G56A | — | 3.03 | 0.2 | 0.3 | ND | ND | ND |
| | | | | | | | (0.57) | | | | | |
| iPS:421182 | — | — | — | — | — | V102F | 1.50 | 0.5 | 0.5 | 5.47 | 2.4 | 1.7 |
| | | | | | | | (0.72) | | | (0.37) | | |
| iPS:421189 | Q27K; | — | — | — | — | — | 0.65 | 1.1 | 1.2 | 7.48 | 1.7 | 1.2 |
| | G30W | | | | | | (0.31) | | | (0.45) | | |
| iPS:421195 | Q27K; | — | — | — | — | — | 0.65 | 1.1 | 1.2 | 5.28 | 1.3 | 1.7 |
| | S32N | | | | | | (0.24) | | | (0.46) | | |
| iPS:421201 | Q27K | — | R93M | — | — | — | 1.70 | 0.4 | 0.5 | 4.63 | 1.4 | 2.0 |
| | | | | | | | (0.37) | | | (0.88) | | |
| iPS:421207 | Q27K | — | — | R31H | — | — | 3.68 | 0.2 | 0.2 | 8.27 | 0.8 | 1.1 |
| | | | | | | | (0.64) | | | (1.17) | | |
| iPS:421211 | Q27K | — | — | F32Y | — | — | 1.67 | 0.4 | 0.5 | 4.25 | 1.6 | 2.1 |
| | | | | | | | (0.32) | | | (0.21) | | |
| iPS:421215 | Q27K | — | — | — | Y53F | — | 1.39 | 0.5 | 0.6 | 3.24 | 2.0 | 2.8 |
| | | | | | | | (0.21) | | | (0.20) | | |
| iPS:421219 | Q27K | — | — | — | G56A | — | 1.19 | 0.6 | 0.7 | ND | ND | ND |
| | | | | | | | (0.51) | | | | | |
| iPS:421223 | Q27K | — | — | — | — | V102F | 0.60 | 1.2 | 1.3 | 6.30 | 1.1 | 1.4 |
| | | | | | | | (0.09) | | | (0.04) | | |
| iPS:421227 | G30W | — | — | — | D54N | — | 0.39 | 1.9 | 2.0 | 5.02 | 1.3 | 1.8 |
| | | | | | | | (0.06) | | | (1.29) | | |
| iPS:421231 | S32N | — | — | — | D54N | — | 0.51 | 1.7 | 1.5 | 6.39 | 1.1 | 1.4 |
| | | | | | | | (0.11) | | | (1.58) | | |
| iPS:421235 | — | — | R93M | — | D54N | — | 1.45 | 0.6 | 0.5 | 0.00 | — | — |
| | | | | | | | (0.18) | | | (0.00) | | |
| iPS:421239 | — | — | — | R31H | D54N | — | 3.87 | 0.2 | 0.2 | 4.28 | 1.6 | 2.1 |
| | | | | | | | (0.10) | | | (0.56) | | |
| iPS:421246 | — | — | — | F32Y | D54N | — | 0.64 | 1.3 | 1.2 | 3.36 | 2.1 | 2.7 |
| | | | | | | | (0.17) | | | (0.50) | | |
| iPS:421253 | — | — | — | — | Y53F; | — | 0.38 | 2.3 | 2.0 | 2.88 | 2.4 | 3.2 |
| | | | | | D54N | | (0.10) | | | (0.17) | | |
| iPS:421260 | — | — | — | — | D54N; | — | 0.43 | 2.0 | 1.8 | 2.85 | 2.4 | 3.2 |
| | | | | | G56A | | (0.16) | | | (0.12) | | |
| iPS:421267 | — | — | — | — | D54N | V102F | 0.23 | 4.0 | 3.5 | 3.51 | 2.0 | 2.6 |
| | | | | | | | (0.01) | | | (0.16) | | |
| iPS:421274 | G30W | — | — | — | G56R | — | 0.61 | 1.5 | 1.3 | 5.12 | 1.4 | 1.8 |
| | | | | | | | (0.04) | | | (0.45) | | |
| iPS:421278 | S32N | — | — | — | G56R | — | 0.90 | 1.0 | 0.9 | 11.96 | 1.1 | 0.8 |
| | | | | | | | (0.14) | | | (6.93) | | |
| iPS:421282 | — | — | R93M | — | G56R | — | 1.12 | 0.8 | 0.7 | 6.99 | 1.9 | 1.3 |
| | | | | | | | (0.25) | | | (0.40) | | |
| iPS:421286 | — | — | — | R31H | G56R | — | 2.92 | 0.3 | 0.3 | 4.11 | 3.2 | 2.2 |
| | | | | | | | (0.50) | | | (0.38) | | |
| iPS:421293 | — | — | — | F32Y | G56R | — | 0.91 | 1.0 | 0.9 | 4.30 | 3.1 | 2.1 |
| | | | | | | | (0.08) | | | (0.42) | | |
| iPS:421300 | — | — | — | — | Y53F; | — | 1.10 | 0.8 | 0.7 | 4.05 | 3.3 | 2.2 |
| | | | | | G56R | | (0.15) | | | (0.88) | | |
| iPS:421307 | — | — | — | — | G56R | V102F | 0.28 | 2.3 | 2.8 | 4.35 | 3.0 | 2.1 |
| | | | | | | | (0.02) | | | (0.04) | | |
| iPS:421314 | G30W | — | — | — | E62R | — | 0.52 | 1.2 | 1.5 | 5.09 | 2.6 | 1.8 |
| | | | | | | | (0.02) | | | (0.49) | | |
| iPS:421318 | S32N | — | — | — | E62R | — | 0.73 | 0.9 | 1.1 | 4.48 | 1.7 | 2.0 |
| | | | | | | | (0.16) | | | (0.28) | | |
| iPS:421322 | — | — | R93M | — | E62R | — | 1.23 | 0.5 | 0.6 | 3.49 | 2.2 | 2.6 |
| | | | | | | | (0.47) | | | (0.28) | | |
| iPS:421326 | — | — | — | R31H | E62R | — | 3.70 | 0.2 | 0.2 | 4.29 | 1.8 | 2.1 |
| | | | | | | | (0.28) | | | (0.81) | | |
| iPS:421333 | — | — | — | F32Y | E62R | — | 1.05 | 0.6 | 0.7 | 1.23 | 6.3 | 7.4 |
| | | | | | | | (0.08) | | | (0.10) | | |
| iPS:421340 | — | — | — | — | Y53F; | — | 0.71 | 0.9 | 1.1 | 1.44 | 5.4 | 6.3 |
| | | | | | E62R | | (0.21) | | | (0.01) | | |
| iPS:421347 | — | — | — | — | G56A; | — | 0.71 | 1.1 | 1.1 | 1.32 | 5.9 | 6.9 |
| | | | | | E62R | | (0.13) | | | (0.45) | | |
| iPS:421354 | — | — | — | — | E62R | V102F | 0.53 | 1.5 | 1.5 | 2.33 | 2.8 | 3.9 |
| | | | | | | | (0.01) | | | (0.67) | | |
| iPS:421361 | G30W | — | — | — | — | V102I | 1.29 | 0.6 | 0.6 | 2.11 | 3.1 | 4.3 |
| | | | | | | | (0.72) | | | (0.11) | | |
| iPS:421365 | S32N | — | — | — | — | V102I | 4.46 | 0.2 | 0.2 | 8.87 | 0.7 | 1.0 |
| | | | | | | | (0.43) | | | (0.57) | | |
| iPS:421369 | — | — | R93M | — | — | V102I | 6.43 | 0.1 | 0.1 | 4.58 | 1.4 | 2.0 |
| | | | | | | | (3.20) | | | (0.38) | | |
| iPS:421373 | — | — | — | R31H | — | V102I | 9.82 | 0.1 | 0.1 | 3.19 | 2.0 | 2.8 |
| | | | | | | | (0.36) | | | (0.13) | | |
| iPS:421380 | — | — | — | F32Y | — | V102I | 3.28 | 0.2 | 0.2 | 3.09 | 2.1 | 2.9 |
| | | | | | | | (0.59) | | | (0.40) | | |
| iPS:421387 | — | — | — | — | Y53F | V102I | 1.78 | 0.6 | 0.4 | 2.45 | 2.7 | 3.7 |
| | | | | | | | (0.32) | | | (0.21) | | |
| iPS:421394 | — | — | — | — | G56A | V102I | 2.36 | 0.4 | 0.3 | 1.65 | 4.0 | 5.5 |
| | | | | | | | (0.20) | | | (0.64) | | |

¹This value represents the fold-increase in IC50 as compared to the IC50 value for the 29G4v10 parental molecule formatted as either a mAb or Fab-Fc fusion protein that was run in parallel with each variant molecule.
²This value represents the fold-increase in IC50 as compared to the average IC50 value for the 29G4v10 parental molecule formatted as either a mAb or Fab-Fc fusion protein
ND = not determined

The greatest improvements in potency were observed when the antibodies had a mutation at position Q27 (Q27K) in the light chain variable region and a mutation at position D54 (D541, D54Q, or D54N) and/or position G56 (G56R or G56N) in the heavy chain variable region. Q27 in the light chain variable region of SEQ ID NO: 52 corresponds to amino acid position 29 in AHo numbering. D54 and G56 in the heavy chain variable region of SEQ ID NO: 191 correspond to amino acid positions 61 and 66 in AHo numbering, respectively. A basic amino acid, such as lysine or arginine, at position Q27 in the light chain variable region presumably provides improved charge complementarity with acidic amino acids Glu120 and Asp121 in the PAC1 ECD (see Zone 1 in FIG. 2A). Hydrophobic residues (e.g. isoleucine) or neutral hydrophilic residues (e.g. glutamine or asparagine) at position D54 in the heavy chain variable region and basic residues (e.g. arginine) or neutral hydrophilic residues (e.g. asparagine) at position G56 in the heavy chain variable region appear to improve the hydrophobic interaction or hydrogen bonding with amino acid residues Asn60 and Ile61 in the PAC1 ECD (see Zone 6 in FIG. 3B).

The top eleven mutants from the MutHC library screen (Table 8) were formatted as aglycosylated IgG1 monoclonal antibodies and monovalent Fab-Fc molecules as described above for production and functional testing in the cAMP assay (Table 13). The modestly improved binding on yeast for this set of variants translated to improved PAC1 receptor blocking function for four out of 11 mAbs and seven out of 11 Fab-Fcs. Nevertheless, as an antibody, the iPS:421873 mutant showed about a 4-fold improvement in PAC1 blocking function compared to parental 29G4v10 antibody. Activity rankings of the mutants were largely consistent across the two formats, with the Fab-Fcs consistently showing weaker function (higher IC50 values) than the mAbs, presumably due to loss of avidity.

TABLE 13
In vitro inhibitory potency of variants from the MutHC library screen

| | | | | | | | | | | Fab-Fc fusion protein | | |
|---|---|---|---|---|---|---|---|---|---|---|---|---|
| | | | | | | | mAb functional activity | | | functional activity | | |
| | | | | | | | (bivalent target binding) | | | (monovalent target binding) | | |
| | Substitutions with respect | | | Substitutions with respect | | | | Fold- | Fold- | | Fold- | Fold- |
| | to 29G4v10 VL sequence | | | to 29G4v10 VH sequence | | | | increase | increase | | increase | increase |
| | (SEQ ID NO: 52) | | | (SEQ ID NO: 191) | | | IC50 | over | over | IC50 | over | over |
| Variant Ab | LC | LC | LC | HC | HC | HC | (SD) | parental | parental | (SD) | parental | parental |
| ID. | CDR1 | CDR2 | CDR3 | CDR1 | CDR2 | CDR3 | in nM | (on plate)¹ | (avg)² | in nM | (on plate)¹ | (avg)² |
| iPS:421855 | — | — | — | R31K; | Y53F; | V102L | 0.55 | 1.8 | 1.4 | 1.30 | 5.1 | 7.0 |
| | | | | F32Y | D54K | | (0.11) | | (0.02) | | | |
| | | | | | G56S | | | | | | | |
| iPS:421861 | — | — | — | F32Y | D54Q | V102L | 1.29 | 0.8 | 0.6 | 3.31 | 2.0 | 2.7 |
| | | | | | | | (0.22) | | | (0.66) | | |
| iPS:421867 | — | — | — | R31H | Y53F; | V102M | 5.55 | 0.2 | 0.1 | 5.41 | 1.2 | 1.7 |
| | | | | | D54S; | | (0.49) | | | (0.25) | | |
| | | | | | G56S | | | | | | | |
| iPS:421873 | — | — | — | R31K | D54R | V102L | 0.25 | 3.9 | 3.1 | 1.27 | 5.2 | 7.1 |
| | | | | | | | (0.04) | | | (0.03) | | |
| iPS:421879 | | | | F32Y | D54S; | V102L; | 1.60 | 0.6 | 0.5 | 2.06 | 3.2 | 4.4 |
| | | | | | G56A | T104S | (0.01) | | | (0.75) | | |
| iPS:421885 | — | — | — | R31H; | Y53F; | V102L | 0.72 | 1.3 | 1.1 | 2.51 | 2.6 | 3.6 |
| | | | | F32Y | D54K; | | (0.14) | | (0.58) | | | |
| | | | | | G56A | | | | | | | |
| iPS:421891 | — | — | — | R31H; | D54R; | V102L | 1.04 | 0.9 | 0.8 | 2.59 | 2.9 | 3.5 |
| | | | | F32Y | G56A | | (0.09) | | | (0.24) | | |
| iPS:421897 | — | — | — | R31H; | Y53F; | V102F | 3.42 | 0.3 | 0.2 | 5.85 | 1.3 | 1.6 |
| | | | | F32Y | D54Y; | | (0.41) | | | (0.71) | | |
| | | | | | G56S | | | | | | | |
| iPS:421903 | — | — | — | R31H | Y53F; | V102F | 2.34 | 0.4 | 0.3 | 4.95 | 1.5 | 1.8 |
| | | | | | D54F | | (0.98) | | | (0.23) | | |
| iPS:421909 | — | — | — | R31H; | Y53F; | V102F | 4.10 | 0.2 | 0.2 | 5.01 | 1.5 | 1.8 |
| | | | | F32Y; | D54M; | | (0.36) | | | (2.10) | | |
| | | | | | G56T | | | | | | | |
| iPS:421915 | — | — | — | R31Y | Y53H; | V102L; | 0.80 | 1.2 | 1.0 | 2.38 | 3.1 | 3.8 |
| | | | | | D54R; | T104S | (0.00) | | | (0.69) | | |
| | | | | | G56T | | | | | | | |

¹This value represents the fold-increase in IC50 as compared to the IC50 value for the 29G4v10 parental molecule formatted as either a mAb or Fab-Fc fusion protein that was run in parallel with each variant molecule.
²This value represents the fold-increase in IC50 as compared to the average IC50 value for the 29G4v10 parental molecule formatted as either a mAb or Fab-Fc fusion protein

A subset of the 29G4v10 mutants from the chain-shuffled libraries (Tables 9 and 10) were converted to aglycosylated IgG1 monoclonal antibodies and tested for functional activity in the cell-based cAMP assay. The results are shown in Table 14 below.

TABLE 14
In vitro inhibitory potency of variants from chain-shuffled
yeast display libraries

| | Variant Ab | Human PAC1 | | Rat PAC1 | |
|---|---|---|---|---|---|
| Clone ID | ID. | IC50 (nM) | SD | IC (nM) | SD |
| 30_D05 | iPS: 480711 | 0.07 | 0.00 | 0.17 | 0.02 |
| 37_F04 | iPS: 480706 | 0.42 | 0.01 | 0.66 | 0.08 |
| 37_A11 | iPS: 480713 | 0.50 | 0.05 | 1.22 | 0.40 |
| 37_H05 | iPS: 480705 | 0.53 | 0.25 | 1.16 | 0.34 |
| 37_B06 | iPS: 480707 | 0.59 | 0.01 | 1.17 | 0.26 |
| 30_H10 | iPS: 480708 | 0.62 | 0.02 | 1.06 | 0.13 |
| 30_F08 | iPS: 480709 | 0.77 | 0.17 | 1.82 | 0.97 |
| 30_E08 | iPS: 480712 | 0.83 | 0.33 | 1.68 | 0.92 |
| 37_E09 | iPS: 480704 | 1.36 | 0.05 | 2.74 | 1.05 |
| 30_A05 | iPS: 480710 | 1.61 | 0.20 | 3.53 | 1.00 |
| 1_D07 | iPS: 480716 | 2.51 | 0.43 | 5.22 | 2.08 |
| 2_C11 | iPS: 480715 | 3.63 | 1.04 | 7.37 | 2.73 |
| 2_G07 | iPS: 480717 | 3.92 | 0.86 | 10.77 | 0.73 |
| 2_B10 | iPS: 480714 | 10.15 | 2.12 | 41.05 | 23.77 |
| 29G4v10 | | 0.75 | | 84.50² | |
| parental | | | | | |
| antibody | | | | | |
| 29G4v22 | | 4.70 | | >1000 | |
| control¹ | | | | | |

¹29G4v22 is another anti-PAC1 neutralizing antibody that shares significant structural similarity with 29G4v10. The VL and VH sequences for 29G4v22 are provided in SEQ ID NOs: 53 and 192 respectively.
²Historical value obtained from a previous assay and included for comparative purposes.

When formatted as monoclonal antibodies (mAbs), 29G4v10 mutants from the second generation chain-shuffled library (Table 10) were more potent antagonists of PAC1 than mutants from the first generation chain-shuffled library (Table 9), suggesting that enhanced PAC1 blocking function is correlated with improved binding and slower binding off-rates, in particular. However, surprisingly, most of the mutants as mAbs were less active than antibody iPS:421873, the most potent mutant from the mutHC library screen (Table 13), despite their presumably enhanced binding to the human PAC1 ECD. Nevertheless, the 30_D05 mutant exhibited about a 10-fold and 67-fold improvement in function as compared to the 29G4v10 parental antibody and 29G4v22 control antibody, respectively. One common feature shared by the highly potent S8_30_D05 and iPS:421873 mAbs is the D54R mutation within HCDR2, which is absent in all of the other less potent mAb mutants. Interestingly, all of the affinity-matured variants exhibit cross-reactivity with the rat PAC1 receptor, in contrast to the 29G4v10 parental antibody and 29G4v22 control antibody (Table 14).

### Example 4. In Vivo Functional Activity of Human PAC1 Antibody Variants

Maxadilan is a vasodilatory peptide and an agonist of the PAC1 receptor. When administered intradermally, maxadilan causes an increase in local dermal blood flow that can be measured by laser Doppler imaging. Inhibition of this effect by PAC1 antagonists (e.g. anti-PAC1 antibodies) can serve as a translational pharmacodynamic model of antagonism of PAC1 biological activity. A subset of the PAC1 variants that exhibited improved in vitro inhibitory potency when formatted as monoclonal antibodies from Example 3 were tested for efficacy in inhibiting PAC1 receptor activation in vivo by using a rat dermal blood flow model.

### In Vivo Pharmacodynamic Model

Naive male Sprague Dawley rats aged at 8-12 weeks at the time of the study were purchased from Charles River Laboratories. All procedures in this example were conducted in compliance with the Animal Welfare Act, the Guide for the Care and Use of Laboratory Animals, and the Office of Laboratory Animal Welfare. Animals were group-housed in non-sterile, ventilated micro-isolator housing in Amgen's Assessment and Accreditation of Laboratory Animal Committee (AAALAC)-accredited facility. Animals had ad libitum access to pelleted feed (Harlan Teklad 2020X, Indianapolis, IN) and water (on-site generated reverse osmosis) via automatic watering system.

The subset of anti-PAC1 antibodies were tested in a rat maxadilan-induced increase in dermal blood flow (MIIBF) pharmacodynamic (PD) model with a laser Doppler imaging. A dosing solution of maxadilan (Bachem, H6734.0500) was prepared fresh daily by diluting maxadilan stock solution (0.5 mg/mL) in 1× phosphate-buffered saline (PBS) to a final concentration of 0.5 μg/mL. All anti-PAC1 antibodies (Abs) were prepared in 10 mM sodium-acetate, 9% sucrose, pH 5.2 (A52Su) at different concentrations, depending on the dose required for the experiment, and administered via a single bolus i.v. injection one day prior to the dermal blood flow (DBF) measurement by the laser Doppler imaging.

A laser Doppler imager (LDI-2, Moor Instruments, Ltd, Wilmington, DE) was used to measure DBF on a shaved patch of skin of the rat abdomen using a low-power laser beam generated by a 633 nm helium-neon bulb. The measurement resolution was 0.2 to 2 mm, with a scanning distance between the instrument aperture and the tissue surface of 30 cm. DBF was measured and expressed as either % change from baseline [100×(individual post-maxadilan flux-individual baseline flux)/individual baseline flux] or % DBF inhibition [Mean of vehicle % change from BL−individual antibody treated rat % change from BL)/Mean of vehicle % change from BL] to quantify the magnitude of the antibody effect.

On the test day, following anesthetization with propofol, the rat's abdominal area was shaved and each animal was placed in a supine position on a temperature-controlled circulating warm-water pad to maintain a stable body temperature during the study. After a 10 to 15 minute stabilization period, a rubber O-ring (0.925 cm inner diameter, 0-Rings West, Seattle, WA) was placed on the rat abdomen (without directly positioning it over a visible blood vessel). After placement of the O-ring on the selected area, a baseline (BL) DBF measurement was taken. After the BL scan, the PAC1 agonist maxadilan was administered by intradermal injection (20 μL of 0.5 μg/mL) at the center of the O-ring. DBF was measured 30 min following maxadilan injection, or 24±1.5 hours following antibody treatment. The O-ring defines the area of interest in which the DBF was analyzed.

All DBF results were expressed as the mean±SEM. A one-way ANOVA followed by Dunnett's Multiple Comparison Test (MCT) was used to assess the statistical significance of PAC1 Ab effects relative to vehicle treatment. A p value of <0.05 was used to determine significance between two groups.

### Single Intravenous (i.v.) Dose Screening Evaluation

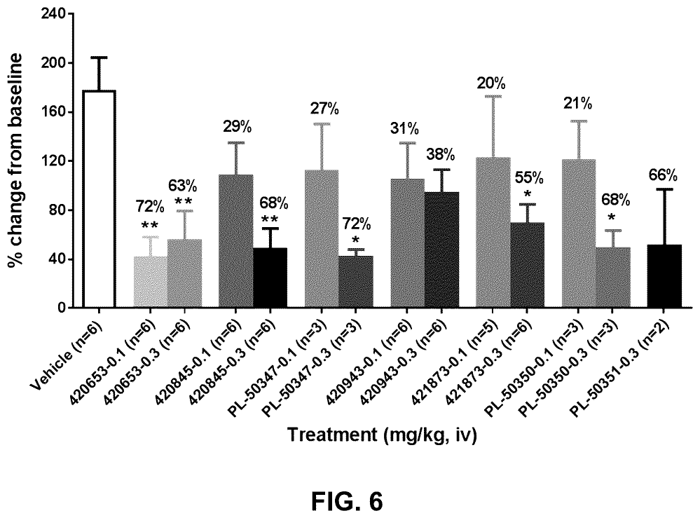

Rats were pretreated with one of 7 different anti-PAC1 antibodies (420653, 420845, 420943, 421873, 420889 (PL-50347), 421091 (PL-50350), and 421051 (PL-50351)) 24 hours prior to maxadilan challenge (20 μl of 0.5 μg/mL) at a dose of 0.1 mg/kg or 0.3 mg/kg, which resulted in a reduction in MIIBF compared to vehicle (A52Su) treated group. At 30 min post-maxadilan treatment, there was a statistically significant inhibition in MIIBF at 0.3 mg/kg compared to the vehicle group for five of the seven antibodies tested (FIG. 6). Terminal serum concentration at 24±1.5 hours for six of the seven antibodies is listed in Table 15 below.

TABLE 15
Terminal serum concentrations for single-injection
(screening) study

Dose (mg/kg)

| Antibody ID. | 0.1 | 0.3 |
|---|---|---|
| 420653 (PL-50345) | 2.0 ± 0.8 | 8.9 ± 2.5 |
| 420845 (PL-50346) | 1.2 ± 0.3 | 6.4 ± 0.8 |
| 420889 (PL-50347) | 5.5 ± 3.1 | 10.7 ± 1.7 |
| 420943 (PL-50348) | 1.6 ± 0.4 | 8.9 ± 1.8 |
| 421873 (PL-50349) | 1.1 ± 0.3 | 4.4 ± 0.4 |
| 421091 (PL-50350) | 1.0 ± 0.1 | 3.0 ± 0.1 |

### Dose-Response Evaluation

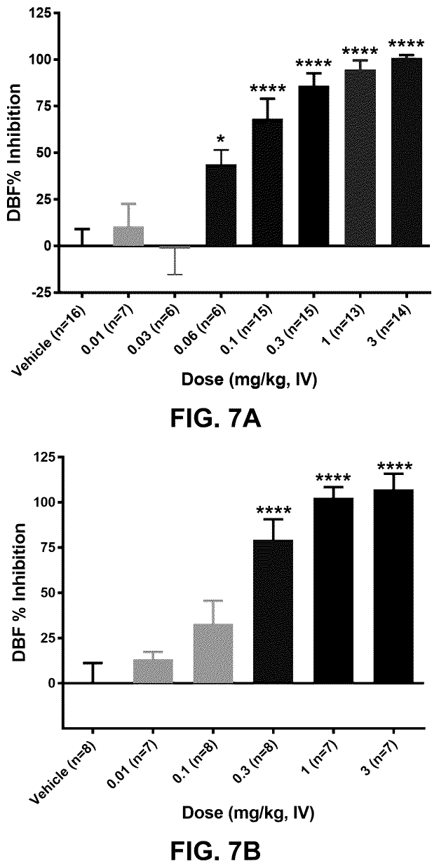

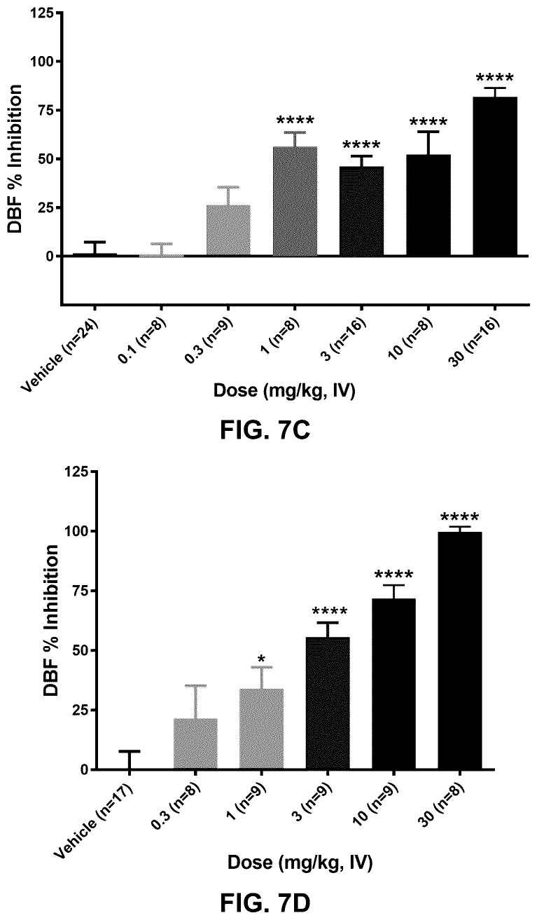

Rats were pretreated with 4 different anti-PAC1 antibodies (420653, 420845, 420943, and 421873) 24 hours prior to maxadilan challenge (20 μl of 0.5 μg/mL) at a dose ranging from 0.01 mg/kg to 30 mg/kg. A dose-dependent reduction of MIIBF compared to vehicle-treated group was observed for each of the four antibodies (FIGS. 7A-7D). Ab 420653 produced a significant effect at a dose as low as 0.06 mg/kg with inhibitory effects of 44%, 68%, 86%, 95%, and 101% at 0.06, 0.1, 0.3, 1 and 3 mg/kg, respectively (FIG. 7A). Ab 420845 produced a significant effect at 0.3, 1 and 3 mg/kg with inhibition of 79% 102% and 107%, respectively (FIG. 7B). Ab 420943 and 421873 were slightly less potent than Ab 420845 and Ab 420653, but still produced a significant inhibitory effect at 1 mg/kg. The % inhibition in DBF for Ab 420943 was 56%, 46%, 52% and 81% at 1, 3, 10, 30 mg/kg, respectively (FIG. 7C). The % inhibition in DBF for Ab 421873 was 34%, 55%, 72% and 100% at 1, 3, 10, and 30 mg/kg, respectively (FIG. 7D).

The results of the experiments described in this example show that antibodies that potently inhibit ligand-induced PAC1 receptor activation in vitro also inhibit PAC1 receptor activation in vivo as assessed by the dermal blood flow assay, a model of PAC1-mediated vasodilation.

### Example 5. Pharmacokinetic Characteristics of Human PAC1 Antibody Variants

Preliminary pharmacokinetic (PK) studies of four anti-PAC1 antibodies (420653, 420845, 420943, and 421873) were conducted with naïve male Sprague-Dawley rats and naïve male cynomolgus monkeys. The 29G4v10 parental antibody or the structurally related 29G4v22 antibody (VL comprising SEQ ID NO: 53 and VH comprising SEQ ID NO: 192) was evaluated as a control. The test antibodies were dosed to study animals by intravenous bolus administration. Blood samples were collected at specified time points post-dose and processed to serum. All serum specimens were stored at approximately −70° C. (±10° C.) until transferred for subsequent analysis.

To measure the amount of test antibody in serum samples from rats and cynomolgus monkeys following dosing, a colorimetric enzyme-linked immunosorbent assay (ELISA) was developed using murine monoclonal antibodies (mAb) directed against human IgG Fc (Amgen, Inc., CA, USA). Microtiter plates were coated with a murine anti-human Fc mAb at 2 μg/mL. The coated microtiter plates were blocked with I-block (Applied Biosystems, CA, USA). Assay standards (STDs) and quality controls (QCs) were prepared by spiking the test antibody into 100% serum from the studied species. STDs, QCs, blank and study samples were diluted 1:30 in assay buffer (1×PBS with 1 M NaCl, 1% BSA and 0.5% Tween 20). Diluted STDs, QCs, blank and study samples were incubated on the coated microtiter plates for 1 h at 25° C. without agitation. After a wash step, a horseradish peroxidase (HRP)-conjugated murine anti-human Fc mAb at 30 ng/mL in assay buffer was added to the microtiter plates and incubated for 1 h at 25° C. without agitation. After a final wash step, a tetramethylbenzidine (TMB) peroxide substrate solution (KPL Inc., MD, USA) was added to the microtiter plates. In the presence of HRP, TMB produced a colorimetric signal that was proportional to the quantity of bound human Fc present in the STDs, QCs and study samples. The color development duration was analyte-dependent and was stopped by addition of 2N sulfuric acid. The optical density (OD) was measured at 450 nm with reference to 650 nm using a SpectraMax 340PC microtiter plate reader (Molecular Devices, CA, USA) and SoftMax Pro software. Assay data were regressed using a logistic (auto-estimate) regression model with a weighting factor of 1/y. The assay dynamic range was from 20 ng/mL to 2000 ng/mL.

For both the rat and cynomolgus monkey PK studies, noncompartmental analysis was performed on individual serum concentration-nominal time data using Phoenix® WinNonlin® (version 6.4; Certara, NJ, USA). Individual concentration values less than the lower limit of quantification (LLOQ, 20 ng/mL) were reported as below the quantitation limit (BQL) and set to zero for the calculation of summary statistics. Mean concentration values less than the LLOQ were not reported or plotted. All concentrations values less than the LLOQ were excluded from the noncompartmental analysis. Nominal doses and nominal sampling times were used for PK analysis. The following PK parameters were estimated:
The initial concentration (C₀) value after intravenous administration was estimated by back extrapolation to time zero using the first 2 observed declining concentration values.
The area under the concentration-time curve from time zero to infinity (AUCinf) was calculated by the linear trapezoidal method.
The terminal-phase half-life (t_{1/2,z}) was calculated as ln 2/λ_{z}. λ_{z} is the first-order rate constant of drug associated with the terminal portion of the curve.
Systemic clearance (CL) was calculated as: CL=Dose/AUCinf after intravenous administration
Volume of distribution at steady state (Vss) was estimated as: Vss=CL×MRTinf (mean residence time from time zero to infinity)

A summary of the in vitro inhibitory potency of the four antibodies as well as for the 29G4v10 parental antibody and the 29G4v22 control antibody against the human PAC1, rat PAC1, and cyno PAC1 is provided in Table 16 below. The antibodies were evaluated for the ability to inhibit ligand-induced (PACAP38 or maxadilan) activation of the PAC1 receptor from different species using the cAMP assay described in Example 3.

TABLE 16
Summary of In Vitro Inhibitory Potency for PAC1 Variant Antibodies

| | Human PAC1 (nM) | | Cyno PAC1 (nM) | | Rat PAC1 (nM) | |
|---|---|---|---|---|---|---|
| | PACAP38 | Maxadilan | PACAP38 | Maxadilan | PACAP38 | Maxadilan |
| Antibody ID | (n = 3) | (n = 3) | (n = 3) | (n = 3) | (n = 3) | (n = 3) |
| 420653 | 0.24 | 0.13 | 0.13 | 0.10 | 0.21 | 0.19 |
| 420845 | 0.09 | 0.09 | 0.08 | 0.09 | 0.11 | 0.07 |
| 420943 | 0.37 | 0.20 | 0.21 | 0.17 | 5.66 | 0.69 |
| 421873 | 0.24 | 0.10 | 0.12 | 0.10 | 0.23 | 0.06 |
| 29G4v22 | 4.70 | 1.60 | 1.11 | 1.00 | >1000 | 580 |
| 29G4v10 | 0.75 | — | 0.33 | — | 84.5 | 8.75 |

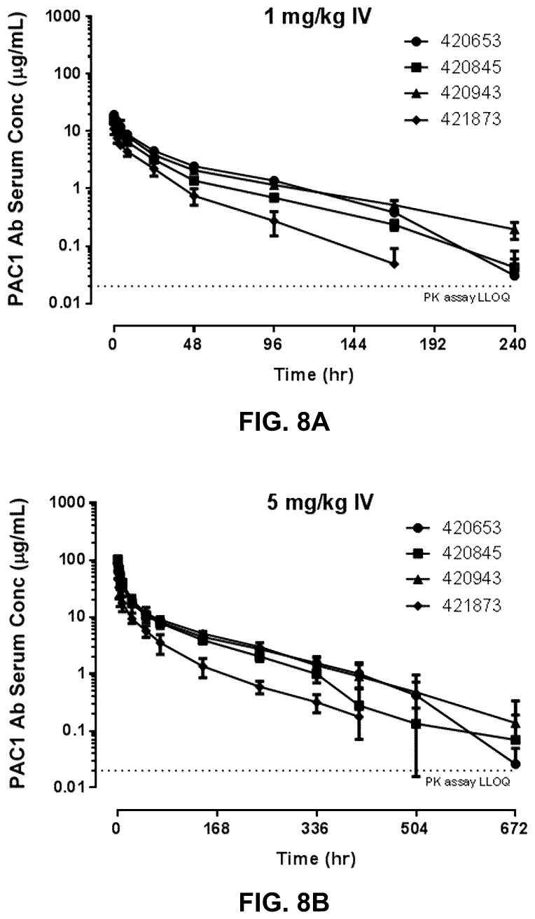

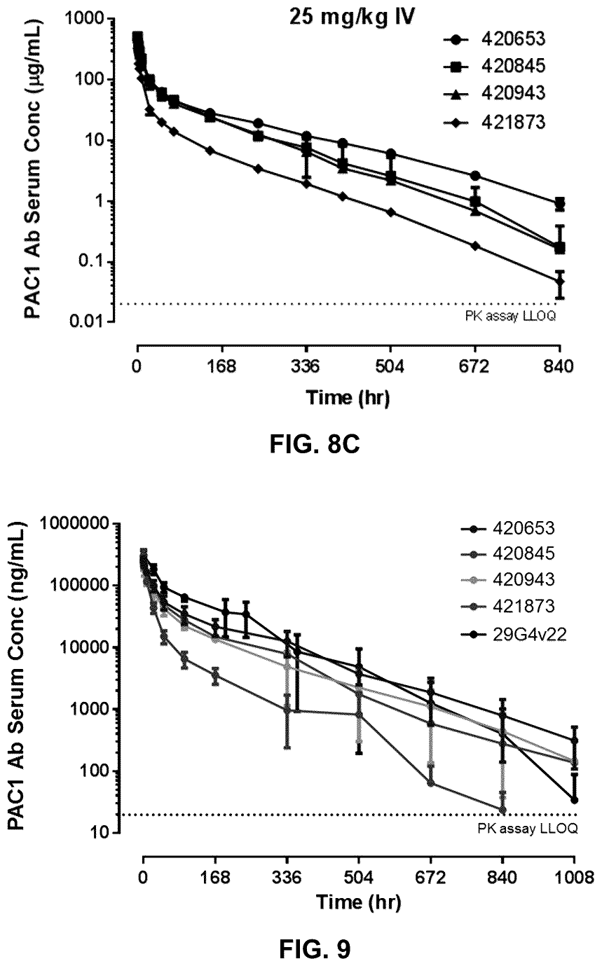

Rats were administered intravenously one of the four antibodies at a dose of 1 mg/kg, 5 mg/kg, and 25 mg/kg. Blood samples were collected at various time points after dosing and antibody concentration was measured in serum samples at each of the time points using the ELISA assay described above. PK parameters for the rat study are summarized in Table 17 below and serum concentration-time profiles for each of the doses are shown in FIGS. 8A-8C.

TABLE 17
Summary of PK Parameters for PAC1 Variant Antibodies in Rats

| | IV Dose | | C₀ | AUC_{0-inf} | Vₛₛ | CL | t_{1/2,z} |
|---|---|---|---|---|---|---|---|
| Antibody ID | (mg/kg) | n | (μg/mL) | (μg * hr/mL) | (mL/kg) | (mL/hr/kg) | (hr) |
| 420653 | 1 | 3 | 19.6 | 467 | 100 | 2.16 | 35.3 |
| | 5 | 3 | 87.1 | 2600 | 218 | 2.05 | 70.5 |
| | 25 | 2 | 462 | 16900 | 236 | 1.48 | 130 |
| 420845 | 1 | 3 | 15.7 | 305 | 141 | 3.28 | 42.2 |
| | 5 | 3 | 104 | 2420 | 171 | 2.08 | 73.2 |
| | 25 | 2 | 525 | 14800 | 179 | 1.69 | 100 |
| 420943 | 1 | 3 | 17.4 | 441 | 129 | 2.28 | 51.3 |
| | 5 | 3 | 101 | 2900 | 184 | 1.75 | 79.3 |
| | 25 | 2 | 490 | 13600 | 191 | 1.83 | 67.9 |
| 421873 | 1 | 3 | 11.2 | 179 | 156 | 5.88 | 27.9 |
| | 5 | 3 | 64.2 | 1100 | 424 | 4.73 | 110 |
| | 25 | 2 | 341 | 5300 | 383 | 4.73 | 96.8 |

Cynomolgus monkeys were administered intravenously one of the four antibodies at a dose of 10 mg/kg. Blood samples were collected at various time points after dosing and antibody concentration was measured in serum samples at each of the time points using the ELISA assay described above. PK parameters for the cynomolgus monkey study are summarized in Table 18 below and the serum concentration-time profiles are shown in FIG. 9. The PK profile of the 29G4v22 antibody is show for comparison.

TABLE 18
Summary of PK Parameters for PAC1 Variant Antibodies in
Cynomolgus Monkeys

| Antibody | | C₀ | AUC_{0-inf} | Vₛₛ | CL | t_{1/2,z} |
|---|---|---|---|---|---|---|
| ID | n | (μg/mL) | (μg * hr/mL) | (mL/kg) | (mL/hr/kg) | (hr) |
| 420653 | 3 | 274 | 14800 | 110 | 0.692 | 134 |
| 420845 | 3 | 314 | 4870 | 126 | 2.09 | 85.3 |
| 420943 | 3 | 240 | 10000 | 126 | 1.00 | 107 |
| 421873 | 3 | 245 | 11800 | 96.7 | 0.849 | 92.6 |
| 29G4v22 | 6 | 312 | 24000 | 43.3 | 0.443 | 70.3 |

At this dose, all four of the PAC1 variant antibodies had a longer serum half-life than the 29G4v22 control antibody. Interestingly, although all four PAC1 variant antibodies exhibited greater in vitro inhibitory potency against the PAC1 receptor as compared with the 29G4v22 control antibody (Table 16), the PK profile for some of the variants was less favorable in cynomolgus monkeys. For example, Ab 420845 had a faster clearance rate and lower overall exposure as compared to the 29G4v22 control antibody. However, Ab 420653 had a comparable PK profile to that for the 29G4v22 control antibody (FIG. 9), suggesting that Ab 420653 could be administered at a lower dose at the same dosing frequency to achieve a similar pharmacological effect.

### Example 6. Yeast Display Affinity Maturation of 19118 Human PAC1 Antibody

To generate additional anti-PAC1 antibodies with improved inhibitory potency, the 19H8 antibody (VH region of SEQ ID NO: 296; VL region of SEQ ID NO: 67) was affinity matured by FACS of yeast-displayed Fab libraries using the methods described in Example 2. The 19H8 antibody is structurally diverse from the 29G4v9, 29G4v10, and 29G4v22 antibodies, but also exhibits very potent neutralizing activity against the human PAC1 receptor.

Because no crystal structural information was available for the PAC1 ECD-19H8 Fab complex initially, a homology model was generated to identify the surface-exposed residues within each CDR loop. Because it is expected that the PAC1 ECD would make direct contacts with surface-exposed CDR residues, it was hypothesized that changing the nature of these contacts or creating new contacts through comprehensive mutagenesis could lead to improved binding of the antibody to the PAC1 ECD. To restrict theoretical diversities of each library to a manageable 10⁶-10⁷, up to five positions per CDR were identified for MIX19 saturation mutagenesis and one separate library per CDR was constructed. Due to concerns regarding the accuracy of modeled CDRH3 loop conformations, the most solvent-exposed residues within this loop could not be reliably identified. Therefore, diversification at eight of the 11 CDR residues within CDRH3 were considered, excluding residues at the beginning and end of the loop. To narrow down the number of positions for saturation mutagenesis to five, the two tryptophans and one phenylalanine within CDRH3 was avoided, as aromatic residues are often critical mediators of protein-protein interactions. In total, six individual-CDR Fab libraries were designed to target 16 heavy chain and 15 light chain CDR residues for diversification, and the constructed libraries oversampled the theoretical diversities by 4.5 to 46-fold. A summary of the targeted positions for each CDR library is provided below:

Heavy chain CDR libraries (amino acid positions relative to SEQ ID NO: 296):
CDRH1 library: Asp27 (located adjacent to CDRH1 in FR1), Ser31, Asn32, Ser33, and Thr35
CDRH2 library: Tyr54, Tyr55, Ser57, Lys58, Ser60, and His62
CDRH3 library: Thr103, Lys105, Gln106, Leu107, and Leu110

Light chain CDR libraries (amino acid positions relative to SEQ ID NO: 67):
CDRL1 library: Ser28, Ser30, Arg31, Tyr32, and Asn34
CDRL2 library: Tyr49 (located adjacent to CDRL2 in FR2), Ala50, Ala51, Ser52, and Ser53
CDRL3 library: Ser91, Tyr92, Ser93, Pro94, and Phe96

The six individual-CDR Fab libraries were enriched for binding to human PAC1 ECD using FACS, increasing the stringency with each successive round by lowering the concentration of the PAC1 ECD used for binding (Round 1: 30 nM PAC1 ECD; Round 2: 0.67 nM PAC1 ECD; and Round 3: 0.2 nM PAC1 ECD). For further affinity improvements, two CDR-shuffled Fab libraries that combined enriched mutations from the individual CDR libraries (one for the heavy chain and one for the light chain) and a final chain-shuffled library that combined enriched mutations from each CDR-shuffled library were also constructed. The CDR-shuffled and chain-shuffled libraries were subjected to selections for PAC1 ECD binding under more stringent conditions using the off-rate binding selection process described in Example 2 and depicted in FIG. 5. The final off-rate sort of the chain-shuffled library suggested that most of the yeast clones within this pool were significantly improved in PAC1 ECD binding compared to parental 19H8 antibody.

Approximately 800 individual yeast clones were screened for improved binding to human PAC1 ECD. Mutant sequences with sequence liabilities (e.g. cysteine anomalies, N-linked glycosylation sites, aspartate isomerization, asparagine deamidation, and tryptophan oxidation sites) were removed. The top ˜200 unique binders were advanced to a secondary screen where they were ranked by binding off-rate and evaluated for non-specific binding. In the secondary screen, >80% of the clones had slower binding off-rates than the parental 19H8 antibody, as measured by a higher association percentage of biotinylated human PAC1 ECD following an overnight competition with unlabeled PAC1 ECD at 30° C. The measurements represent lower limits on the improvement in off-rate, as biotinylated PAC1 ECD fully dissociated from parental 19H8 after only one hour of competition at 30° C. Because none of the screened clones exhibited binding to the ECDs of unrelated PD1 and GIPR receptors, to further narrow down the pool of clones, additional sequence filters were applied to remove mutants whose CDRs contained furin cleavage sites, additional tryptophan residues, and more covariance violations than the parental 19H8 antibody. An additional binding assay using a biotinylated human VPAC2 ECD to determine the degree of non-specific binding to human VPAC2, a structurally related receptor to human PAC1 receptor, was used to rank clones. The top 20 mutants exhibiting the slowest binding off-rates to human PAC1 ECD and the least amount of human VPAC2 binding among the yeast clones that entered the secondary screen are listed in Table 19 below. Higher percentage association of PAC1 ECD binding indicates that the mutant Fabs have a slower off-rate.

TABLE 19
Top Improved Binders from Yeast Display Libraries for 19H8 Parental Antibody

| | Substitutions with respect | | | Substitutions with respect | | | % human | |
|---|---|---|---|---|---|---|---|---|
| | to 19H8 VH sequence | | | to 19H8 VL sequence | | | PAC1 ECD | Biotinylated |
| | (SEQ ID NO: 296) | | | (SEQ ID NO: 67) | | | association | VPAC2 ECD |
| | HC | | | | LC | | after off-rate | Binding |
| | FR1- | HC | HC | LC | FR2- | LC | competition | (fluorescence |
| Variant Ab ID. | CDR1 | CDR2 | CDR3 | CDR1 | CDR2 | CDR3 | at 30° C. | units) |
| iPS:448202 | S31N; | S57G; | K105N; | — | Y49F; | S91A; | 86% | 562 |
| | N32R; | S60K | L107D | | A51G; | Y92I; | | |
| | S33L | | | | S52Q; | S93G; | | |
| | | | | | S53R | P94M; | | |
| | | | | | | F96Y | | |
| iPS:449375 | S31N; | S57G; | K105D; | S28Y; | S52H; | S91A; | 80% | 506 |
| | N32R; | S60K | L107D | S30V | S53H | Y92I; | | |
| | S33L | | | | | S93Q; | | |
| | | | | | | P94E; | | |
| | | | | | | F96Y | | |
| iPS:448083 | N32R; | S57G; | K105E; | S28T; | Y49F; | S91A; | 75% | 573 |
| | S33Q | S60K | L107D | S30V | A51G; | Y92I; | | |
| | | | | | S52Q; | S93I; | | |
| | | | | | S53R | P94N; | | |
| | | | | | | F96Y | | |
| iPS:452128 | S31N; | S57G; | T103M; | S28Y; | S52N; | S91A; | 74% | 535 |
| | N32K; | S60K | K105N; | S30V | S53M | Y92I; | | |
| | S33Q | | L107N | | | S93N; | | |
| | | | | | | P94Q; | | |
| | | | | | | F96Y | | |
| iPS:448195 | S31N; | S57G; | T103M; | S28K; | S52N; | Y92I; | 70% | 577 |
| | N32K; | S60K | K105N; | S30A; | S53M | S93Q; | | |
| | S33Q | | L107N | N34V | | P94Q; | | |
| | | | | | | F96Y | | |
| iPS:448466 | S31N; | S57G; | K105I; | — | Y49F; | S91A; | 66% | 510 |
| | N32K; | K58Q; | Q106G; | | A51G; | Y92I; | | |
| | S33Q | S60K | L107D; | | S52Q; | S93Q; | | |
| | | | L110M | | S53R | P94Q; | | |
| | | | | | | F96Y | | |
| iPS:448689 | S31N; | S57G; | K105N; | S28Y; | A51S; | S91A; | 66% | 596 |
| | N32R; | S60K | L107D | S30V | S52Y; | Y92I; | | |
| | S33L | | | | S53N | S93M; | | |
| | | | | | | P94A; | | |
| | | | | | | F96Y | | |
| iPS:449034 | S31N; | S57G; | K105I; | S28P; | A51G; | S91A; | 65% | 519 |
| | N32K; | S60K | L107D | S30A; | S52R; | Y92I; | | |
| | S33Q | | | R31Q | S53Y | S93Q; | | |
| | | | | | | P94N; | | |
| | | | | | | F96Y | | |
| iPS:448568 | N32R; | S57G; | T103M; | — | A51G; | S91A; | 63% | 620 |
| | S33Y | S60K | K105A; | | S52L; | Y92I; | | |
| | | | Q106G; | | S53Y | S93Q; | | |
| | | | L107D; | | | P94T; | | |
| | | | L110M | | | F96Y | | |
| iPS:448924 | N32R; | S57G; | T103M; | S28P; | Y49F; | S91A; | 61% | 596 |
| | S33Y; | K58Q; | K105N; | N34S | A51G; | Y92I; | | |
| | | S60K | L107N | | S52Q; | P94I; | | |
| | | | | | S53R | F96Y | | |
| iPS:448752 | S31N; | S57G; | T103M; | S28Q; | A51S; | S91A; | 59% | 544 |
| | N32K; | S60K; | K105N; | S30A | S52Y; | Y92I; | | |
| | S33Q | | L107N; | | S53N | S93I; | | |
| | | | | | | P94Q; | | |
| | | | | | | F96Y | | |
| iPS:448772 | S31N; | S57G; | T103M; | — | A51S; | S91A; | 59% | 594 |
| | N32K; | S60K | K105S; | | S52Y; | Y92I; | | |
| | S33Q | | Q106G; | | S53N | S93Q; | | |
| | | | L107D | | | P94N; | | |
| | | | | | | F96Y | | |
| iPS:448117 | N32H; | S57G; | T103M; | S28T; | Y49F; | Y92I; | 58% | 496 |
| | S33V | S60K | K105S; | S30V | A51G; | S93Q; | | |
| | | | Q106E; | | S52Q; | P94T; | | |
| | | | L107D | | S53R | F96Y | | |
| iPS:448788 | N32R; | S57G; | T103M; | — | A51G; | S91A; | 56% | 565 |
| | S33Q | S60K | K105Q; | | S52R; | Y92I; | | |
| | | | Q106G; | | S53I | S93I; | | |
| | | | L107N | | | P94N; | | |
| | | | | | | F96Y | | |
| iPS:448593 | S31N; | S57G; | K105I; | — | A51S; | S91A; | 54% | 544 |
| | N32H; | S60K | L107D | | S52Y; | Y92I; | | |
| | S33Q; | | | | S53N | S93Q; | | |
| | | | | | | P94N; | | |
| | | | | | | F96Y | | |
| iPS:448238 | N32R; | S57G; | T103Q; | S28R; | Y49F; | S91A; | 53% | 527 |
| | S33D | S60K | K105N; | S30A; | A51G; | Y92I; | | |
| | | | Q106E; | | S53I | S93Q; | | |
| | | | L107D | | | P94N; | | |
| | | | | | | F96Y | | |
| iPS:448901 | S31N; | S57G; | T103R; | — | A51S; | Y92I; | 51% | 546 |
| | N32R; | S60K; | K105E; | | S52Y; | S93Q; | | |
| | S33L | | Q106G; | | S53N; | P94Q; | | |
| | | | L107D; | | | F96Y | | |
| | | | L110F | | | | | |
| iPS:448225 | S31N; | S57G; | K105N; | S28M | A50V; | Y92I; | 50% | 627 |
| | N32R; | S60K | L107D | | S53R | S93Q; | | |
| | S33L; | | | | | P94N; | | |
| | | | | | | F96Y | | |
| iPS:448730 | S31N; | S57G; | T103V; | S28M; | A51S; | S91A; | 50% | 525 |
| | N32R; | S60K | K105I; | S30A | S52Y; | Y92I; | | |
| | S33L | | Q106G; | | S53N | S93I; | | |
| | | | L107N | | | P94N; | | |
| | | | | | | F96Y | | |
| iPS:449027 | S31N; | S57G; | T103M; | S28Y; | A50G; | Y92I; | 49% | 521 |
| | N32K; | S60K | K105N; | S30V; | S52R; | S93Q; | | |
| | S33Q | | L107N | | S53N | P94T; | | |
| | | | | | | F96Y | | |
| 19H8 Wild-Type | — | — | — | — | — | — | 20% | 528 |

The improved affinity variants were produced by recombinant expression methods as complete bivalent monoclonal antibodies and/or as monovalent Fab-Fc fusions (e.g. Fab region fused to dimeric IgG Fc region) and evaluated for in vitro functional activity in the cell-based cAMP assay described in Example 3. The results of the functional assay are shown in Table 20 below.

TABLE 20
In vitro inhibitory potency of 19H8 variant antibodies and Fab-Fc fusion proteins

| | Substitutions with respect | | | Substitutions with respect | | | | |
|---|---|---|---|---|---|---|---|---|
| | to 19H8 VH sequence | | | to 19H8 VL sequence | | | PAC1 functional cAMP assay | |
| | (SEQ ID NO: 296) | | | (SEQ ID NO: 67) | | | | Fab-Fc fusion |
| | HC | | | | LC | | mAb IC50 in nM | protein IC50 in nM |
| | FR1- | HC | HC | LC | FR2- | LC | (bivalent | (monovalent |
| Variant Ab ID. | CDR1 | CDR2 | CDR3 | CDR1 | CDR2 | CDR3 | target binding) | target binding) |
| iPS:448202 | S31N; | S57G; | K105N; | — | Y49F; | S91A; | 0.32 | 1.25 |
| | N32R; | S60K | L107D | | A51G; | Y92I; | | |
| | S33L | | | | S52Q; | S93G; | | |
| | | | | | S53R | P94M; | | |
| | | | | | | F96Y | | |
| iPS:449375 | S31N; | S57G; | K105D; | S28Y; | S52H; | S91A; | 0.95 | 3.48 |
| | N32R; | S60K | L107D | S30V | S53H | Y92I; | | |
| | S33L | | | | | S93Q; | | |
| | | | | | | P94E; | | |
| | | | | | | F96Y | | |
| iPS:448083 | N32R; | S57G; | K105E; | S28T; | Y49F; | S91A; | — | 3.74 |
| | S33Q | S60K | L107D | S30V | A51G; | Y92I; | | |
| | | | | | S52Q; | S93I; | | |
| | | | | | S53R | P94N; | | |
| | | | | | | F96Y | | |
| iPS:452128 | S31N; | S57G; | T103M; | S28Y; | S52N; | S91A; | 0.38 | 0.18 |
| | N32K; | S60K | K105N; | S30V | S53M | Y92I; | | |
| | S33Q | | L107N | | | S93N; | | |
| | | | | | | P94Q; | | |
| | | | | | | F96Y | | |
| iPS:448195 | S31N; | S57G; | T103M; | S28K; | S52N; | Y92I; | 0.08 | 0.26 |
| | N32K; | S60K | K105N; | S30A; | S53M | S93Q; | | |
| | S33Q | | L107N | N34V | | P94Q; | | |
| | | | | | | F96Y | | |
| iPS:448466 | S31N; | S57G; | K105I; | — | Y49F; | S91A; | 1.10 | 1.36 |
| | N32K; | K58Q; | Q106G; | | A51G; | Y92I; | | |
| | S33Q | S60K | L107D; | | S52Q; | S93Q; | | |
| | | | L110M | | S53R | P94Q; | | |
| | | | | | | F96Y | | |
| iPS:448689 | S31N; | S57G; | K105N; | S28Y; | A51S; | S91A; | 0.20 | 0.62 |
| | N32R; | S60K | L107D | S30V | S52Y; | Y92I; | | |
| | S33L | | | | S53N | S93M; | | |
| | | | | | | P94A; | | |
| | | | | | | F96Y | | |
| iPS:449034 | S31N; | S57G; | K105I; | S28P; | A51G; | S91A; | 1.42 | 1.58 |
| | N32K; | S60K | L107D | S30A; | S52R; | Y92I; | | |
| | S33Q | | | R31Q | S53Y | S93Q; | | |
| | | | | | | P94N; | | |
| | | | | | | F96Y | | |
| iPS:448075 | S33H | S57G; | K105D; | — | Y49F; | S91A; | 2.10 | 3.69 |
| | | S60K | L107D | | A51G; | Y92I; | | |
| | | | | | S52Q; | S93V; | | |
| | | | | | S53R | P94Q; | | |
| | | | | | | F96Y | | |
| iPS:448924 | N32R; | S57G; | T103M; | S28P; | Y49F; | S91A; | 0.45 | 0.21 |
| | S33Y | K58Q; | K105N; | N34S | A51G; | Y92I; | | |
| | | S60K | L107N | | S52Q; | P94I; | | |
| | | | | | S53R | F96Y | | |
| iPS:448752 | S31N; | S57G; | T103M; | S28Q; | A51S; | S91A; | — | 0.30 |
| | N32K | S60K | K105N; | S30A | S52Y; | Y92I; | | |
| | S33Q; | | L107N; | | S53N | S93I; | | |
| | | | | | | P94Q; | | |
| | | | | | | F96Y | | |
| iPS:448772 | S31N; | S57G; | T103M; | — | A51S; | S91A; | 0.89 | 2.01 |
| | N32K; | S60K | K105S; | | S52Y; | Y92I; | | |
| | S33Q | | Q106G; | | S53N | S93Q; | | |
| | | | L107D | | | P94N; | | |
| | | | | | | F96Y | | |
| iPS:448117 | N32H; | S57G; | T103M; | S28T; | Y49F; | Y92I; | 1.26 | 2.27 |
| | S33V | S60K | K105S; | S30V | A51G; | S93Q; | | |
| | | | Q106E; | | S52Q; | P94T; | | |
| | | | L107D | | S53R | F96Y | | |
| iPS:448788 | N32R; | S57G; | T103M; | — | A51G; | S91A; | 0.09 | 0.37 |
| | S33Q | S60K | K105Q; | | S52R; | Y92I; | | |
| | | | Q106G; | | S53I | S93I; | | |
| | | | L107N | | | P94N; | | |
| | | | | | | F96Y | | |
| iPS:448593 | S31N; | S57G; | K105I; | — | A51S; | S91A; | 2.79 | 20.67 |
| | N32H; | S60K | L107D | | S52Y; | Y92I; | | |
| | S33Q | | | | S53N | S93Q; | | |
| | | | | | | P94N; | | |
| | | | | | | F96Y | | |
| iPS:448238 | N32R; | S57G; | T103Q; | S28R; | Y49F; | S91A; | 0.65 | 1.63 |
| | S33D | S60K | K105N; | S30A | A51G; | Y92I; | | |
| | | | Q106E; | | S53I | S93Q; | | |
| | | | L107D | | | P94N; | | |
| | | | | | | F96Y | | |
| iPS:448901 | S31N; | S57G; | T103R; | — | A51S; | Y92I; | 0.15 | 0.41 |
| | N32R; | S60K | K105E; | | S52Y | S93Q; | | |
| | S33L | | Q106G; | | S53N | P94Q; | | |
| | | | L107D; | | | F96Y | | |
| | | | L110F | | | | | |
| iPS:448655 | S31N; | Y55F; | T103M; | S28Y | A51S; | S91A; | — | 1.34 |
| | N32K | S57R; | K105S; | S30V | S52Y; | Y92I; | | |
| | S33Q | K58T; | Q106E; | | S53N | S93Q; | | |
| | | S60K | L107D | | | P94Q; | | |
| | | | | | | F96Y | | |
| iPS:448730 | S31N; | S57G; | T103V; | S28M; | A51S; | S91A; | 0.08 | 0.14 |
| | N32R; | S60K | K105I; | S30A | S52Y; | Y92I; | | |
| | S33L | | Q106G; | | S53N | S93I; | | |
| | | | L107N | | | P94N; | | |
| | | | | | | F96Y | | |
| iPS:449027 | S31N; | S57G; | T103M; | S28Y; | A50G; | Y92I; | — | 1.85 |
| | N32K; | S60K | K105N; | S30V | S52R; | S93Q; | | |
| | S33Q | | L107N | | S53N | P94T; | | |
| | | | | | | F96Y | | |
| 3574 | S31N; | S57G; | T103M; | — | — | — | 0.19 | — |
| | N32K; | S60K | K105N; | | | | | |
| | S33Q | | L107N | | | | | |
| 3575 | — | — | — | S28Q; | A51S; | S91A; | 0.14 | — |
| | | | | S30A; | S52Y; | Y92I; | | |
| | | | | | S53N | S93I; | | |
| | | | | | | P94Q; | | |
| | | | | | | F96Y | | |
| 19H8 Wild-Type | — | — | — | — | — | — | 0.40¹ | 8.21 |

¹Historical value obtained from a previous assay and included for comparative purposes.

When formatted as Fab-Fc fusion proteins, the 19H8 variants exhibited a 2-fold to 58-fold increase in inhibitory potency as compared with the parental 19H8 Fab-Fc fusion protein. Many of the 19H8 variants were more potent than the 29G4v10 variants when formatted as Fab-Fc fusion proteins (compare results in Table 20 with those in Table 12 for Fab-Fc fusion proteins). When formatted as bivalent monoclonal antibodies, the 19H8 variants also exhibited potent human PAC1 neutralizing activity with IC50 values in the single digit nanomolar or picomolar range.

### Example 7. In Vivo Functional Activity of a Human PAC1 Antibody Variant in Cynomolgus Monkeys

To evaluate target engagement in vivo with anti-PAC1 antibody 420653, the ability of the antibody to inhibit maxadilan-induced increase in dermal blood flow in cynomolgus monkeys was assessed. Maxadilan is a selective agonist of the PAC1 receptor and can activate the receptor in rodents, cynomolgus monkeys, and humans. As described in Example 4, intradermal administration of maxadilan causes an increase in local dermal blood flow that can be measured by laser Doppler imaging. Inhibition of this effect by anti-PAC1 antibodies can serve as a translational pharmacodynamic model of antagonism of PAC1 biological activity.

Male cynomolgus monkeys between 5 and 8 years of age were used for the study. A dosing solution of maxadilan (Bachem, H6734.0500) was prepared fresh daily by diluting maxadilan stock solution (0.5 mg/mL) in 1× phosphate-buffered saline (PBS). Anti-PAC1 antibody 420653 or 29G4v22, which was tested as a control, were prepared in 10 mM sodium-acetate, 9% sucrose, 0.01% Tween-80, pH 5.2 (A52SuT) at different concentrations, depending on the dose required for the experiment, and administered via intravenous (i.v.) infusion.

A laser Doppler imager (LDI2-IR, Moor Instruments, Ltd, Wilmington, DE) was used to measure dermal blood flow (DBF) on a shaved patch of skin of either the ventral forearm or medial thigh using a low-power laser beam generated by a 633 nm helium-neon bulb. The infra-red wavelength was combined with a visible red aiming beam to give a higher weighting to blood flow in the deeper dermis (0.6 to 1 mm). The incident light was scattered by static tissue and moving blood. Moving blood in the microvasculature caused a Doppler light shift. The shifted light from moving blood and the non-shifted light from tissue was then directed onto 2 square-law detectors. The detected intensity fluctuations were then processed to give parameters of flux (proportional to tissue blood flow) and concentration (proportional to the concentration of moving blood cells). The measurement resolution was 0.2 to 2 mm, with a scanning distance between the instrument aperture and the tissue surface of 20 to 100 cm.

DBF was measured as Flux (relative units) and expressed as % change from baseline 30 min post-maxadilan administration [100×(individual post-maxadilan flux-individual baseline flux)/individual baseline flux]. Flux units from two separate O-rings were averaged together for each test session. The inhibitory effect of the anti-PAC1 antibodies on maxadilan-induced increase in blood flow (MIIBF) from individual animals was expressed as % inhibition [100×(Mean of day 0% change from baseline−individual antibody treated animal % change from baseline)/Mean of day 0% change from baseline].

Following anesthesia and stabilization of vital signs for 15 to 20 minutes, rubber O-rings (0.925 cm inner diameter, 0-Rings West, Seattle, WA) were placed about 0.6 to 1 cm apart from each other on the pre-shaved skin of the ventral forearm or medial thigh without direct positioning over a visible vessel. The limb used for each testing session was pre-determined, with a different limb used on different testing days of the study. After placement of the O-rings on the skin, a baseline measurement was taken. After the baseline scan, a maxadilan solution of 1 ng in 20 μL vehicle (PBS) was injected intradermally at the center of the O-rings. The O-rings served as a region of interest during data analysis.

Prior to anti-PAC1 antibody administrations, the time course of MIIBF was obtained at Day 0. The maxadilan dose of 1 ng was selected based on the result of a previous maxadilan dose-response in cynomolgus monkeys. After a pre-maxadilan baseline DBF measurement, the time course of DBF response to maxadilan intradermal injection was assessed by obtaining laser Doppler scans at 5, 10, 15, 20, 25, and 30 minutes post-maxadilan application. To identify maxadilan responders and eliminate non-responders from inclusion in the study, a pre-screen procedure prior to antibody administration was implemented. Animals were considered maxadilan responders and included in the study if they had a change in DBF flow ≥60 flux units (average of post maxadilan DBF at 30 minutes−average of baseline).

Twenty-four cynomolgus monkeys identified as maxadilan responders received either antibody 420653 at a dose of 0.1 mg/kg or 3 mg/kg or antibody 29G4v22 at a dose of 10 mg/kg at Day 0. The antibodies were administered via i.v. infusion to the saphenous vein at a rate of 1 mL/min by a syringe infusion pump. Post-maxadilan DBF measurements were taken on Days 2, 4, 7, 10, 14, 21, 28, and 36 using different limbs. In each case, DBF was measured by laser Doppler imaging for 30 minutes following 1 ng maxadilan intradermal injection. Whole blood samples for pharmacokinetic (PK) analysis were collected at multiple time points including prior to antibody dosing, 30 min, 1, 2, 4, 7, 10, 14, 21, 28, 35 or 36 and 42 days post-antibody treatment.

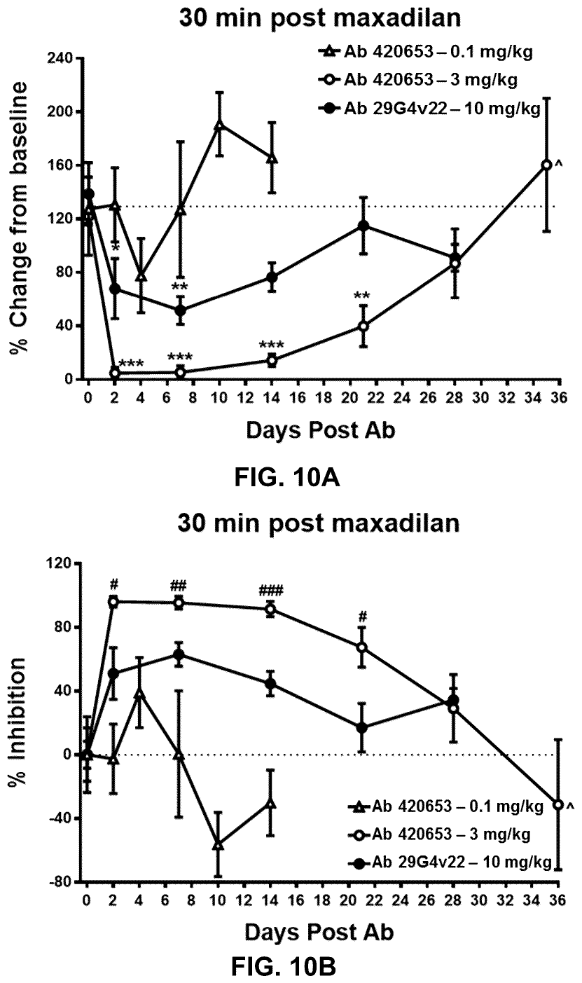

As shown in FIG. 10A, antibody 420653 at 3 mg/kg, but not 0.1 mg/kg, significantly prevented MIIBF on Day 2, Day 7, Day 14, and Day 21 (post-antibody treatment) compared to Day 0 (pre-antibody treatment) (n=8 animals/group; Bonferroni adjusted p-value=0.0087). Antibody 29G4v22 at 10 mg/kg also significantly prevented MIIBF on Day 2 and Day 7 (n=8 animals/group)(FIG. 10A). The maximum inhibitory effect of antibody 420653 at 3 mg/kg was on Day 2 with 96% inhibition, whereas at 0.1 mg/kg, antibody 420653 produced a maximum inhibition of 39% on Day 4 that was not statistically significant (FIG. 10B). Antibody 29G4v22 at 10 mg/kg produced a maximum inhibition of 63% on Day 7 (FIG. 10B). Comparison of antibody 420653 (3 mg/kg) and antibody 29G4v22 (10 mg/kg) at the same time points showed significant differences on Day 2, Day 7, Day 14 and Day 21 (FIG. 10B).

The antibody serum concentrations for antibody 420653 and antibody 29G4v22 at days when DBF measurements were taken are shown below in Table 21. The low limit of quantitation (LLOQ) or below quantitation level (BQL) was 10 ng/mL for antibody 420653 and 50 ng/mL for antibody 29G4v22.

TABLE 21
Serum Concentrations of Antibodies 420653 and 29G4v22 in
Cynomolgus Monkey Following Antibody Treatment

| | | | Serum | | |
|---|---|---|---|---|---|
| Dose | Dose | Time Point | Concentration (μg/L) | | |
| Treatment | (mg/kg) | (Days) | Mean | SD | n |
| Antibody | 0.1 | 2 | 687 | 96.6 | 8 |
| 420653 | | 4 | 315 | 57.3 | 8 |
| | | 7 | 95.9 | 33.5 | 8 |
| | | 10* | 30.1 | 10.9 | 4 |
| | | 14* | LLOQ | — | 0 |
| Antibody | 3 | 2 | 36,200 | 5,010 | 8 |
| 420653 | | 7 | 14,100 | 2,170 | 8 |
| | | 14 | 5350 | 2,140 | 8 |
| | | 21* | 2,210 | 1,800 | 7 |
| | | 36* | 1,480 | — | 1 |
| Antibody | 10 | 2 | 113,000 | 28,400 | 8 |
| 29G4v22 | | 7 | 38,600 | 8,230 | 8 |
| | | 14 | 13,900 | 6,360 | 8 |
| | | 21 | 4,850 | 2,790 | 8 |
| | | 28 | 1,990 | 1,490 | 8 |

*Fewer PK data points due to some samples with LLOQ/BQL

Overall, these results demonstrate that antibody 420653 significantly attenuated MIIBF in cynomolgus monkeys when administered at a dose of 3 mg/kg. The inhibitory effect of antibody 420653 at 3 mg/kg on MIIBF was more robust than antibody 29G4v22 at a higher dose of 10 mg/kg.

All publications, patents, and patent applications discussed and cited herein are hereby incorporated by reference in their entireties. It is understood that the disclosed invention is not limited to the particular methodology, protocols and materials described as these can vary. It is also understood that the terminology used herein is for the purposes of describing particular embodiments only and is not intended to limit the scope of the appended claims.

Those skilled in the art will recognize, or be able to ascertain using no more than routine experimentation, many equivalents to the specific embodiments of the invention described herein. Such equivalents are intended to be encompassed by the following claims.

What is claimed:

1. An isolated antibody or antigen-binding fragment thereof that specifically binds to human pituitary adenylate cyclase-activating polypeptide type I receptor (PAC1)(SEQ ID NO: 1), wherein the antibody or antigen-binding fragment thereof comprises: (i) a light chain variable region in which the amino acid at position 29 according to AHo numbering is a basic amino acid that interacts with amino acids Glu120 or Asp121 of human PAC1, and (ii) a heavy chain variable region in which the amino acid at position 61 according to AHo numbering is a hydrophobic, basic, or neutral hydrophilic amino acid that interacts with amino acids Asn60 or Ile61 of human PAC1, and wherein the antibody or antigen-binding fragment thereof inhibits PACAP-induced activation of human PAC1 with an IC50 less than 500 pM as measured by a cell-based cAMP assay.

2. The isolated antibody or antigen-binding fragment thereof of claim 1, wherein the amino acid at position 29 in the light chain variable region is lysine or arginine.

3. The isolated antibody or antigen-binding fragment thereof of claim 1, wherein the amino acid at position 61 in the heavy chain variable region is isoleucine, valine, leucine, glutamine, asparagine, arginine, or lysine.

4. The isolated antibody or antigen-binding fragment thereof of claim 1, wherein the amino acid at position 66 according to AHo numbering in the heavy chain variable region of the antibody or antigen-binding fragment thereof is a basic or neutral hydrophilic amino acid that interacts with amino acids Asn60 or Ile61 of human PAC1.

5. The isolated antibody or antigen-binding fragment thereof of claim 4, wherein the amino acid at position 66 in the heavy chain variable region is glutamine, asparagine, arginine, or lysine.

6. The isolated antibody or antigen-binding fragment thereof claim 1, wherein the antibody or antigen-binding fragment thereof binds to human PAC1 at an epitope that further comprises one or more amino acids selected from Asp59, Arg116, Asn117, Thr119, Gly122, Trp123, Ser124, Glu125, Pro126, Phe127, Pro128, His129, Tyr130, Phe131, Asp132, and Gly135 of SEQ ID NO: 1.

7. The isolated antibody or antigen-binding fragment thereof of claim 1, wherein the light chain variable region comprises a sequence that is at least 90% identical to the sequence of SEQ ID NO: 52 and the heavy chain variable region comprises a sequence that is at least 90% identical to the sequence of SEQ ID NO: 191.

8. An isolated antibody or antigen-binding fragment thereof that specifically binds to human PAC1, wherein the antibody or antigen-binding fragment thereof comprises a light chain variable region comprising complementarity determining regions CDRL1, CDRL2, and CDRL3 and a heavy chain variable region comprising complementarity determining regions CDRH1, CDRH2, and CDRH3, wherein CDRL1 comprises a sequence selected from SEQ ID NOs: 5-25; CDRL2 comprises a sequence selected from SEQ ID NOs: 26-35; CDRL3 comprises a sequence selected from SEQ ID NOs: 36-51; CDRH1 comprises a sequence selected from SEQ ID NOs: 88-105; CDRH2 comprises a sequence selected from SEQ ID NOs: 106-170; and CDRH3 comprises a sequence selected from SEQ ID NOs: 171-190.

9. The isolated antibody or antigen-binding fragment thereof of claim 8, wherein:
(a) CDRL1, CDRL2, and CDRL3 have the sequence of SEQ ID NOs: 7, 26, and 36, respectively, and CDRH1, CDRH2, and CDRH3 have the sequence of SEQ ID NOs: 88, 108, and 171, respectively;
(b) CDRL1, CDRL2, and CDRL3 have the sequence of SEQ ID NOs: 7, 26, and 36, respectively, and CDRH1, CDRH2, and CDRH3 have the sequence of SEQ ID NOs: 88, 116, and 171, respectively;
(c) CDRL1, CDRL2, and CDRL3 have the sequence of SEQ ID NOs: 5, 26, and 36, respectively, and CDRH1, CDRH2, and CDRH3 have the sequence of SEQ ID NOs: 88, 134, and 171, respectively;
(d) CDRL1, CDRL2, and CDRL3 have the sequence of SEQ ID NOs: 5, 26, and 36, respectively, and CDRH1, CDRH2, and CDRH3 have the sequence of SEQ ID NOs: 92, 145, and 174, respectively;
(e) CDRL1, CDRL2, and CDRL3 have the sequence of SEQ ID NOs: 7, 26, and 36, respectively, and CDRH1, CDRH2, and CDRH3 have the sequence of SEQ ID NOs: 88, 108, and 172, respectively;
(f) CDRL1, CDRL2, and CDRL3 have the sequence of SEQ ID NOs: 5, 26, and 36, respectively, and CDRH1, CDRH2, and CDRH3 have the sequence of SEQ ID NOs: 88, 128, and 172, respectively;
(g) CDRL1, CDRL2, and CDRL3 have the sequence of SEQ ID NOs: 7, 26, and 36, respectively, and CDRH1, CDRH2, and CDRH3 have the sequence of SEQ ID NOs: 88, 134, and 171, respectively;
(h) CDRL1, CDRL2, and CDRL3 have the sequence of SEQ ID NOs: 12, 26, and 36, respectively, and CDRH1, CDRH2, and CDRH3 have the sequence of SEQ ID NOs: 88, 153, and 171, respectively;
(i) CDRL1, CDRL2, and CDRL3 have the sequence of SEQ ID NOs: 12, 26, and 36, respectively, and CDRH1, CDRH2, and CDRH3 have the sequence of SEQ ID NOs: 88, 154, and 171, respectively;
(j) CDRL1, CDRL2, and CDRL3 have the sequence of SEQ ID NOs: 13, 26, and 36, respectively, and CDRH1, CDRH2, and CDRH3 have the sequence of SEQ ID NOs: 88, 155, and 171, respectively;
(k) CDRL1, CDRL2, and CDRL3 have the sequence of SEQ ID NOs: 25, 31, and 42, respectively, and CDRH1, CDRH2, and CDRH3 have the sequence of SEQ ID NOs: 98, 168, and 190, respectively;
(l) CDRL1, CDRL2, and CDRL3 have the sequence of SEQ ID NOs: 20, 30, and 44, respectively, and CDRH1, CDRH2, and CDRH3 have the sequence of SEQ ID NOs: 100, 168, and 182, respectively;
(m) CDRL1, CDRL2, and CDRL3 have the sequence of SEQ ID NOs: 17, 33, and 42, respectively, and CDRH1, CDRH2, and CDRH3 have the sequence of SEQ ID NOs: 99, 168, and 187, respectively;
(n) CDRL1, CDRL2, and CDRL3 have the sequence of SEQ ID NOs: 23, 31, and 50, respectively, and CDRH1, CDRH2, and CDRH3 have the sequence of SEQ ID NOs: 97, 167, and 178, respectively;
(o) CDRL1, CDRL2, and CDRL3 have the sequence of SEQ ID NOs: 17, 31, and 44, respectively, and CDRH1, CDRH2, and CDRH3 have the sequence of SEQ ID NOs: 98, 168, and 189, respectively;
(p) CDRL1, CDRL2, and CDRL3 have the sequence of SEQ ID NOs: 17, 27, and 39, respectively, and CDRH1, CDRH2, and CDRH3 have the sequence of SEQ ID NOs: 100, 168, and 182, respectively;
(q) CDRL1, CDRL2, and CDRL3 have the sequence of SEQ ID NOs: 18, 31, and 46, respectively, and CDRH1, CDRH2, and CDRH3 have the sequence of SEQ ID NOs: 98, 168, and 179, respectively;
(r) CDRL1, CDRL2, and CDRL3 have the sequence of SEQ ID NOs: 17, 28, and 40, respectively, and CDRH1, CDRH2, and CDRH3 have the sequence of SEQ ID NOs: 98, 168, and 179, respectively;
(s) CDRL1, CDRL2, and CDRL3 have the sequence of SEQ ID NOs: 18, 30, and 43, respectively, and CDRH1, CDRH2, and CDRH3 have the sequence of SEQ ID NOs: 100, 168, and 182, respectively; or
(t) CDRL1, CDRL2, and CDRL3 have the sequence of SEQ ID NOs: 22, 28, and 49, respectively, and CDRH1, CDRH2, and CDRH3 have the sequence of SEQ ID NOs: 102, 169, and 182, respectively.

10. The isolated antibody or antigen-binding fragment thereof of claim 8, wherein the light chain variable region comprises (i) a sequence that is at least 90% identical to a sequence selected from SEQ ID NOs: 54-66 and 68-87, (ii) a sequence that is at least 95% identical to a sequence selected from SEQ ID NOs: 54-66 and 68-87, or (iii) a sequence selected from SEQ ID NOs: 54-66 and 68-87.

11. The isolated antibody or antigen-binding fragment thereof of claim 8, wherein the heavy chain variable region comprises (i) a sequence that is at least 90% identical to a sequence selected from SEQ ID NOs: 191-312, (ii) a sequence that is at least 95% identical to a sequence selected from SEQ ID NOs: 191-312, or (iii) a sequence selected from SEQ ID NOs: 191-312.

12. The isolated antibody or antigen-binding fragment thereof of claim 8, wherein:
(a) the light chain variable region comprises the sequence of SEQ ID NO: 54 and the heavy chain variable region comprises the sequence of SEQ ID NO: 194;
(b) the light chain variable region comprises the sequence of SEQ ID NO: 54 and the heavy chain variable region comprises the sequence of SEQ ID NO: 203;
(c) the light chain variable region comprises the sequence of SEQ ID NO: 55 and the heavy chain variable region comprises the sequence of SEQ ID NO: 228;
(d) the light chain variable region comprises the sequence of SEQ ID NO: 55 and the heavy chain variable region comprises the sequence of SEQ ID NO: 274;
(e) the light chain variable region comprises the sequence of SEQ ID NO: 54 and the heavy chain variable region comprises the sequence of SEQ ID NO: 214;
(f) the light chain variable region comprises the sequence of SEQ ID NO: 55 and the heavy chain variable region comprises the sequence of SEQ ID NO: 240;
(g) the light chain variable region comprises the sequence of SEQ ID NO: 54 and the heavy chain variable region comprises the sequence of SEQ ID NO: 228;
(h) the light chain variable region comprises the sequence of SEQ ID NO: 62 and the heavy chain variable region comprises the sequence of SEQ ID NO: 282;
(i) the light chain variable region comprises the sequence of SEQ ID NO: 62 and the heavy chain variable region comprises the sequence of SEQ ID NO: 283;
(j) the light chain variable region comprises the sequence of SEQ ID NO: 63 and the heavy chain variable region comprises the sequence of SEQ ID NO: 284;
(k) the light chain variable region comprises the sequence of SEQ ID NO: 85 and the heavy chain variable region comprises the sequence of SEQ ID NO: 312;
(l) the light chain variable region comprises the sequence of SEQ ID NO: 72 and the heavy chain variable region comprises the sequence of SEQ ID NO: 300;
(m) the light chain variable region comprises the sequence of SEQ ID NO: 81 and the heavy chain variable region comprises the sequence of SEQ ID NO: 307;
(n) the light chain variable region comprises the sequence of SEQ ID NO: 78 and the heavy chain variable region comprises the sequence of SEQ ID NO: 296;
(o) the light chain variable region comprises the sequence of SEQ ID NO: 83 and the heavy chain variable region comprises the sequence of SEQ ID NO: 310;
(p) the light chain variable region comprises the sequence of SEQ ID NO: 87 and the heavy chain variable region comprises the sequence of SEQ ID NO: 300;
(q) the light chain variable region comprises the sequence of SEQ ID NO: 74 and the heavy chain variable region comprises the sequence of SEQ ID NO: 297;
(r) the light chain variable region comprises the sequence of SEQ ID NO: 68 and the heavy chain variable region comprises the sequence of SEQ ID NO: 297;
(s) the light chain variable region comprises the sequence of SEQ ID NO: 71 and the heavy chain variable region comprises the sequence of SEQ ID NO: 300; or
(t) the light chain variable region comprises the sequence of SEQ ID NO: 77 and the heavy chain variable region comprises the sequence of SEQ ID NO: 304.

13. The isolated antibody or antigen-binding fragment thereof of claim 8, wherein the antibody or antigen-binding fragment thereof is a monoclonal antibody or antigen-binding fragment thereof.

14. The isolated antibody or antigen-binding fragment thereof of claim 13, wherein the monoclonal antibody or antigen-binding fragment thereof is a humanized antibody or antigen-binding fragment thereof or a fully human antibody or antigen-binding fragment thereof.

15. The isolated antibody or antigen-binding fragment thereof of claim 13, wherein the monoclonal antibody comprises a light chain that comprises a human kappa constant region or a human lambda constant region.

16. The isolated antibody or antigen-binding fragment thereof of claim 15, wherein the human kappa constant region comprises the sequence of SEQ ID NO: 318 or SEQ ID NO: 319.

17. The isolated antibody or antigen-binding fragment thereof of claim 15, wherein the human lambda constant region comprises the sequence of SEQ ID NO: 315.

18. The isolated antibody or antigen-binding fragment thereof of claim 13, wherein the monoclonal antibody is a human IgG1, IgG2, IgG3, or IgG4 antibody.

19. The isolated antibody or antigen-binding fragment thereof of claim 18, wherein the monoclonal antibody is an aglycosylated human IgG1 antibody.

20. The isolated antibody or antigen-binding fragment thereof of claim 19, wherein the monoclonal antibody comprises a mutation at amino acid position N297 according to EU numbering in its heavy chain.

21. The isolated antibody or antigen-binding fragment thereof of claim 20, wherein the mutation is N297G.

22. The isolated antibody or antigen-binding fragment thereof of claim 21, wherein the monoclonal antibody further comprises R292C and V302C mutations according to EU numbering in its heavy chain.

23. The isolated antibody or antigen-binding fragment thereof of claim 19, wherein the monoclonal antibody comprises a heavy chain constant region that comprises the sequence of SEQ ID NO: 324 or SEQ ID NO: 325.

24. An isolated antibody that specifically binds to human PAC1, wherein the antibody comprises a light chain and a heavy chain, wherein:
(a) the light chain comprises the sequence of SEQ ID NO: 506 and the heavy chain comprises the sequence of SEQ ID NO: 521;
(b) the light chain comprises the sequence of SEQ ID NO: 506 and the heavy chain comprises the sequence of SEQ ID NO: 522;
(c) the light chain comprises the sequence of SEQ ID NO: 504 and the heavy chain comprises the sequence of SEQ ID NO: 523;
(d) the light chain comprises the sequence of SEQ ID NO: 504 and the heavy chain comprises the sequence of SEQ ID NO: 524;
(e) the light chain comprises the sequence of SEQ ID NO: 506 and the heavy chain comprises the sequence of SEQ ID NO: 525;
(f) the light chain comprises the sequence of SEQ ID NO: 504 and the heavy chain comprises the sequence of SEQ ID NO: 526;
(g) the light chain comprises the sequence of SEQ ID NO: 506 and the heavy chain comprises the sequence of SEQ ID NO: 523;
(h) the light chain comprises the sequence of SEQ ID NO: 507 and the heavy chain comprises the sequence of SEQ ID NO: 527;
(i) the light chain comprises the sequence of SEQ ID NO: 507 and the heavy chain comprises the sequence of SEQ ID NO: 528;
(j) the light chain comprises the sequence of SEQ ID NO: 508 and the heavy chain comprises the sequence of SEQ ID NO: 529;
(k) the light chain comprises the sequence of SEQ ID NO: 510 and the heavy chain comprises the sequence of SEQ ID NO: 531;
(l) the light chain comprises the sequence of SEQ ID NO: 511 and the heavy chain comprises the sequence of SEQ ID NO: 532;
(m) the light chain comprises the sequence of SEQ ID NO: 512 and the heavy chain comprises the sequence of SEQ ID NO: 533;
(n) the light chain comprises the sequence of SEQ ID NO: 513 and the heavy chain comprises the sequence of SEQ ID NO: 534;
(o) the light chain comprises the sequence of SEQ ID NO: 514 and the heavy chain comprises the sequence of SEQ ID NO: 535;
(p) the light chain comprises the sequence of SEQ ID NO: 515 and the heavy chain comprises the sequence of SEQ ID NO: 535;
(q) the light chain comprises the sequence of SEQ ID NO: 516 and the heavy chain comprises the sequence of SEQ ID NO: 532;
(r) the light chain comprises the sequence of SEQ ID NO: 517 and the heavy chain comprises the sequence of SEQ ID NO: 536;
(s) the light chain comprises the sequence of SEQ ID NO: 509 and the heavy chain comprises the sequence of SEQ ID NO: 532; or
(t) the light chain comprises the sequence of SEQ ID NO: 518 and the heavy chain comprises the sequence of SEQ ID NO: 530.

25. A pharmaceutical composition comprising the isolated antibody or antigen-binding fragment thereof of claim 1 and a pharmaceutically acceptable excipient.

26. A pharmaceutical composition comprising the isolated antibody or antigen-binding fragment thereof of claim 8 and a pharmaceutically acceptable excipient.

27. An isolated polynucleotide that encodes the isolated antibody or antigen-binding fragment thereof of claim 8.

28. An expression vector comprising the polynucleotide of claim 27.

29. A host cell comprising the expression vector of claim 28.

30. A method of producing an antibody or antigen-binding fragment thereof that specifically binds to human PAC1 comprising culturing the host cell of claim 29 under conditions that allow expression of the antibody or antigen-binding fragment thereof; and recovering the antibody or antigen-binding fragment thereof from the culture medium or host cell.

31. A method for inhibiting vasodilation in a patient in need thereof comprising administering to the patient an effective amount of the antibody or antigen-binding fragment thereof of claim 1.

32. A method for inhibiting activation of human PAC1 receptor in a patient having a headache condition comprising administering to the patient an effective amount of the antibody or antigen-binding fragment thereof of claim 1.

33. A method for treating or preventing a headache condition in a patient in need thereof comprising administering to the patient an effective amount of the antibody or antigen-binding fragment thereof of claim 1.

34. The method of claim 33, wherein the headache condition is migraine or cluster headache.

35. The method of claim 34, wherein the migraine is episodic migraine or chronic migraine.

36. The method of claim 33, wherein the antibody or antigen-binding fragment thereof is administered to the patient as a prophylactic treatment.

37. The method of claim 33, further comprising administering to the patient a second headache therapeutic agent.

38. The method of claim 37, wherein the second headache therapeutic agent is an acute headache therapeutic agent.

39. The method of claim 38, wherein the acute headache therapeutic agent is a 5HT_{1B}, 5HT_{1D} and/or 5HT_{1F} serotonin receptor agonist.

40. The method of claim 39, wherein the serotonin receptor agonist is a triptan or ergotamine.

41. The method of claim 38, wherein the acute headache therapeutic agent is a non-steroidal anti-inflammatory drug or an opioid.

42. The method of claim 37, wherein the second headache therapeutic agent is a prophylactic headache therapeutic agent.

43. The method of claim 42, wherein the prophylactic headache therapeutic agent is an antiepileptic, a beta-blocker, an anti-depressant, onabotulinum toxin A, or a CGRP pathway antagonist.

44. The method of claim 43, wherein the CGRP pathway antagonist is a human CGRP receptor antagonist.

45. The method of claim 44, wherein the human CGRP receptor antagonist is erenumab.
